SEQUENCE LISTING (14 sequences)

SEQ ID NO: 1 - protein, 598 aa, Artificial Sequence
  Synthetic polypeptide
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 2 - protein, 302 aa, Artificial Sequence
  Synthetic polypeptide
MEFGLLSEAEARSPALSLSDAGTPHPQLPEHGCKGQEHSDSEKASASLPGGSPEDGSLKKKQRRQRTHFTSQQLQELEATFQRNRYPDMSTREEIAVWTNLTEARVRVWFKNRRAKWRKRERSQQAELCKGSFAAPLGGLVPPYEEVYPGYSYGNWPPKALAPPLAAKTFPFAFNSVNVGPLASQPVFSPPSSIAASMVPSAAAAPGTVPGPGALQGLGGGPPGLAPAAVSSGAVSCPYASAAAAAAAAASSPYVYRDPCNSSLASLRLKAKQHASFSYPAVHGPPPAANLSPCQYAVERPV

SEQ ID NO: 3 - protein, 528 aa, Artificial Sequence
  Synthetic polypeptide
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 4 - protein, 514 aa, Artificial Sequence
  Synthetic polypeptide
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 5 - DNA, 21 bp, Artificial Sequence
  Synthetic polynucleotide
caactacagcacaggctacga

SEQ ID NO: 6 - DNA, 26 bp, Artificial Sequence
  Synthetic polynucleotide
gcatctgaatgtcttctaccttaatg

SEQ ID NO: 7 - DNA, 18 bp, Artificial Sequence
  Synthetic polynucleotide
gcaactggccgcccaagg

SEQ ID NO: 8 - DNA, 18 bp, Artificial Sequence
  Synthetic polynucleotide
aggccccacgttgaccga

SEQ ID NO: 9 - DNA, 23 bp, Artificial Sequence
  Synthetic polynucleotide
gaagtccacctgctaaggaagaa

SEQ ID NO: 10 - DNA, 23 bp, Artificial Sequence
  Synthetic polynucleotide
tcactggagacacatgtacacag

SEQ ID NO: 11 - DNA, 20 bp, Artificial Sequence
  Synthetic polynucleotide
tccaacctttcctggcccag

SEQ ID NO: 12 - DNA, 20 bp, Artificial Sequence
  Synthetic polynucleotide
gcatgaagggcaggaggaat

SEQ ID NO: 13 - DNA, 19 bp, Artificial Sequence
  Synthetic polynucleotide
tgggaggccatcacattgt

SEQ ID NO: 14 - DNA, 21 bp, Artificial Sequence
  Synthetic polynucleotide
aatccagcaggtcagcaaaga

(19) United States
(12) Patent Application Publication
(10) Pub. No.: US 2023/0063230 A1
(43) Pub. Date: Mar. 2, 2023

(54) NURR1 RECEPTOR MODULATORS

(71) Applicants: The Regents of the University of California, Oakland, CA (US); ShangPharma Innovation Inc., South San Francisco, CA (US)

(72) Inventors: Pamela M. England, Pacifica, CA (US); Matthew P. Jacobson, San Francisco, CA (US); Richard Beresis, San Francisco, CA (US)

(73) Assignees: The Regents of the University of California, Oakland, CA (US); ShangPharma Innovation Inc., South San Francisco, CA (US)

(21) Appl. No.: 17/431,111

(22) PCT Filed: Feb. 19, 2020
(86) PCT No.: PCT/US2020/018890

§ 371 Date: Aug. 13, 2021

Related U.S. Application Data
(60) Provisional application No. 62/807,642, filed on Feb. 19, 2019.

(51) Int. Cl. C07D 249/04; C07D 231/12; C07D 403/12; C07F 9/165; C07D 413/12; C07D 257/04; C07C 233/22; C07C 233/11; C07D 265/32; C07C 311/06; C07C 309/70; C07C 321/20; C07C 231/02; C07D 401/04; C07D 403/04; C07F 5/02; C07D 217/02; C07C 67/03; C07C 303/38; C07C 231/12; C07D 413/06; C07D 209/30; C07C 303/22; C07C 247/06; C07C 303/28; C07F 7/08; C07D 409/04

(52) U.S. Cl. CPC C07D 249/04; C07D 409/04; C07D 403/12; C07F 9/165; C07D 413/12; C07D 257/04; C07C 233/22; C07C 233/11; C07D 265/32; C07C 311/06; C07C 309/70; C07C 321/20; C07C 231/02; C07D 401/04; C07D 403/04; C07F 5/027; C07D 217/02; C07C 67/03; C07C 303/38; C07C 231/12; C07D 413/06; C07D 209/30; C07C 303/22; C07C 247/06; C07C 303/28; C07F 7/0812; C07D 231/12

(57) ABSTRACT

Described herein, inter alia, are Nurr1 receptor modulators and uses thereof. In an aspect is provided a method for treating a disease associated with dysregulation and/or degeneration of dopaminergic neurons in the central nervous system of a subject in need thereof, the method including administering to the subject in need thereof a therapeutically effective amount of a compound described herein.

### CROSS-REFERENCES TO RELATED APPLICATIONS

This application claims the benefit of U.S. Provisional Application No. 62/807,642, filed Feb. 19, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. R01 NS108404 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-637001WO_Sequence_Listing ST25.txt, created Jan. 14, 2020, 19,310 bytes, machine format IBM-PC, MS Windows operating system, is hereby incorporated by reference.

### BACKGROUND

Over one million Americans are currently living with Parkinson's disease (PD), and approximately 60,000 new cases are diagnosed each year. In an estimated 90% of PD patients, the cause of the disease is unknown, having no clear genetic or environmental origin. The most pronounced neuropathological feature of PD is the progressive degeneration of dopaminergic neurons in the substantia nigra pars compacta and the consequent reduction in dopamine levels in the striatum, which manifest as impairments in motor function (e.g., rigidity, tremor, bradykinesia). Although the molecular basis for idiopathic PD remains incompletely understood, it has been proposed to include oxidative stress, mitochondrial dysfunction, and dysregulation of dopamine homeostasis. Currently, there are no available treatments that stop or even slow the progression of PD. Existing therapeutics relieve PD symptoms by increasing dopaminergic signaling through one of three mechanisms: (1) increasing dopamine levels by augmenting the amount of its biosynthetic precursor, L-DOPA; (2) blocking the breakdown of dopamine by inhibiting its metabolic enzymes (monoamine oxidase (MAO), COMT); (3) mimicking the activity of dopamine by directly agonizing dopamine receptors. However, these drugs only partially alleviate symptoms and can have significant side effects, especially as the disease progresses. New types of therapeutics are desperately needed to combat both the symptoms and progression of PD. Disclosed herein, inter alfa, are solutions to these and other problems in the art.

### BRIEF SUMMARY

In an aspect is provided a compound having the formula

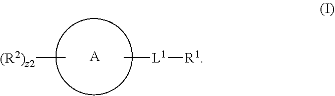

Ring A is aryl or heteroaryl.

L¹ is L¹⁰¹-L¹⁰²-L¹⁰³.

L¹⁰¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, L¹⁰⁴-L¹⁰⁵, L¹⁰⁴, —NH-L¹⁰⁵, or L¹⁰⁴-CH₂-L¹⁰⁵.

L¹⁰² is a bond, —S(O)₂—, —N(R¹⁰²)—, —O—, —S—, —C(O)—, —C(O)N(R¹⁰²)—, —N(R¹⁰²)C (O)—, —N(R¹⁰²)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹⁰³ is a bond, —S(O)₂—, —N(¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹⁰⁴ is a bond, —O—, —NH—, —S—, —S(O)₂—, —C(O)—, —NHC(O)—, —C(O)NH—, —OC(O)—, —C(O)O—, substituted or unsubstituted alkylene, or substituted or unsubstituted heteroalkylene.

L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene.

R¹⁰¹, R¹⁰², and R¹⁰³ are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is hydrogen, halogen, —CX¹₃, —CHX¹₂, —CH₂X¹, —OCX¹₃, —OCH₂X¹, —OCHX¹₂, —CN, —SOₙ₁R^{1D},—SOᵥ₁NR^{1A}R^{1B}, NHC(O)NR^{1A}R^{1B}, —N(O)ₘ₁, —NR^{1A}R^{1B}, —C(O)R^{1C}, —C(O)OR^{1C}, —SC(O)R^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SSR^{1D}, —SiR^{1A}R^{1B}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

E is an electrophilic moiety.

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{2C}, —C(O)OR^{2C}, —C(O)NR^{2A}R^{2B}, —OR^{2D},—SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl.

n1 and n2 are independently an integer from 0 to 4.

m1, m2, v1, and v2 are independently 1 or 2.

X¹ and X² are independently —F, —Cl, —Br, or —I.

z2 is an integer from 0 to 5.

In an aspect is provided pharmaceutical composition including a compound described herein and a pharmaceutically acceptable excipient.

In an aspect is provided a method for treating a disease associated with dysregulation and/or degeneration of dopaminergic neurons in the central nervous system of a subject in need thereof, the method including administering to the subject in need thereof a therapeutically effective amount of a compound described herein.

In an aspect is provided a method of modulating the level of activity of Nurr1 in a subject in need thereof, the method including administering to the subject in need thereof a therapeutically effective amount of a compound described herein.

In an aspect is provided a method of increasing the level of activity of Nurr1 in a cell, the method including contacting the cell with a compound described herein.

In an aspect is provided a method of increasing the level of dopamine in a cell, the method including contacting the cell with a compound described herein.

### BRIEF DESCRIPTION OF THE DRAWINGS

FIGS. 1A-1C. Crystal structures of Nurr1-screening hit complexes reveal two different ligand binding sites and receptor conformations. FIG. 1A: Structures of screening hits 19.49 and 10.25. FIG. 1B: Structure of 19.49 screening hit covalently bound to Cys566. FIG. 1C: Structure of 10.25 screening hit covalently bound to Cys566.

FIGS. 2A-2B. Compounds 85 (FIG. 2A) and 87 (FIG. 2B) both bind to the Nurr1 ligand binding domain with high nanomolar affinity. Binding measured by microscale thermophoresis.

FIGS. 3A-3B. Compounds 85 (FIG. 3A) and 87 (FIG. 3B) stimulate the transcription of Nurr1 target genes in MN9D cells. Gene expression was normalized to the Hprt.

FIGS. 4A-4D. Reaction schemes for select compounds.

### DETAILED DESCRIPTION

I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, monoor polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2, 5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different.

Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

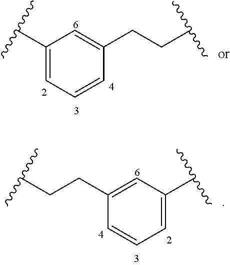 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkyl ene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted aryl ene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each sub stituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted aryl ene, and/or substituted heteroarylene) is substituted with at least one size-limited substituent group, wherein if the substituted moiety is substituted with a plurality of size-limited sub stituent groups, each size-limited sub stituent group may optionally be different. In embodiments, if the substituted moiety is substituted with a plurality of size-limited sub stituent groups, each size-limited sub stituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)or, as (D)or (L)for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)and (S)—, or (D)and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³Cor ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N— hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N— hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N— hydroxysuccinimide moiety) is covalently attached to the second bioconjugate reactive group (e.g., an amine).

Useful bioconjugate reactive moieties used for bioconjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido or maleimide groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold, or react with maleimides;

(h) amine or sulfhydryl groups (e.g., present in cysteine), which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g., phosphines) to form, for example, phosphate diester bonds;

(n) azides coupled to alkynes using copper catalyzed cycloaddition click chemistry; and

(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple 10³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is a cancer.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis,

Sjogren's syndrome,vasculitis, glomerulonephritis, auto-immune thyroiditis, Behcet' s disease, Crohn's disease, ulcerative colitis, bullous pemphigoid, sarcoidosis, ichthyosis, Graves ophthalmopathy, inflammatory bowel disease, Addison's disease, Vitiligo,asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, hi stiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed—Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compounds described herein can be co-administered with conventional neurodegenerative disease treatments including, but not limited to, Parkinson's disease treatments such as levodopa, carbidopa, selegiline, amantadine, donepezil, galanthamine, rivastigmine, tacrine, dopamine agonists (e.g., bromocriptine, pergolide, pramipexole, ropinirole), anticholinergic drugs (e.g., trihexyphenidyl, benztropine, biperiden, procyclidine), and catechol-O-methyl-transferase inhibitors (e.g., tolcapone, entacapone).

The compounds described herein can also be co-administered with conventional anti-inflammatory disease treatments including, but not limited to, analgesics (e.g., acetaminophen, duloxetine), nonsteroidal anti-inflammatory drugs (e.g., aspirin, ibuprofen, naproxen, diclofenac), corticosteroids (e.g., prednisone, betamethasone, cortisone, dexamethasone, hydrocortisone, methylprednisolone, prednisolone), and opoids (e.g., codeine, fentanyl, hydrocodone, hydromorphone, morphine, meperidine, oxycodone).

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC₄₁₂, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-aacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin—N triacetate; lanreotide;leinamycin;lenograstim;lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-b enzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L.sub.2), interferon alfa-2a; interferon alfa-2b; interferon alfa-nl; interferon alfa-n3; interferon beta-la; interferon gamma-lb; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.TM (i.e. paclitaxel), Taxotere.TM, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005(Indena), Cryptophycin 52 (i.e., LY-355703), AC-7739 (Ajinomoto, i.e., AVE-8063A and CS-39.HC₁), AC-7700 (Ajinomoto, i.e., AVE-8062, AVE-8062A, CS-39-L—Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin Al (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3—IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3—IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER², anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., neurodegenerative disease, cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). For example, a neurodegenerative disease associated with a protein aggregate may be a neurodegenerative disease that results (entirely or partially) from aberrant protein aggregation or a neurodegenerative disease wherein a particular symptom of the disease is caused (entirely or partially) by aberrant protein aggregation. As used herein, what is described as being associated with a disease, if a causative agent, could be a target for treatment of the disease. For example, a neurodegenerative disease associated with aberrant protein aggregation or a protein aggregate associated neurodegenerative disease, may be treated with a protein aggregate modulator.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “Nurr1” or “NR⁴A2” refers to the protein that in humans is encoded by the NR⁴A2 gene. Nurr1 is a nuclear receptor and plays a key role in the maintenance of the dopaminergic system of the brain. The term “Nurr1” may refer to the nucleotide sequence or protein sequence of human NR⁴A2 (e.g., Entrez 4929, Uniprot P43354, RefSeq NM_006186.3, or RefSeq NP_006177.1). In embodiments, Nurr1 has the following amino acid sequence:

(SEQ ID NO: 1)
MPCVQAQYGSSPQGASPASQSYSYHSSGEYSSDFLTPEFVKFSMDLTNTE
ITATTSLPSFSTFMDNYSTGYDVKPPCLYQMPLSGQQSSIKVEDIQMHNY
QQHSHLPPQSEEMMPHSGSVYYKPSSPPTPTTPGFQVQHSPMWDDPGSLH
NFHQNYVATTHMIEQRKTPVSRLSLFSFKQSPPGTPVSSCQMRFDGPLHV
PMNPEPAGSHHVVDGQTFAVPNPIRKPASMGFPGLQIGHASQLLDTQVPS
PPSRGSPSNEGLCAVCGDNAACQHYGVRTCEGCKGFFKRTVQKNAKYVCL
ANKNCPVDKRRRNRCQYCRFQKCLAVGMVKEVVRTDSLKGRRGRLPSKPK
SPQEPSPPSPPVSLISALVRAHVDSNPAMTSLDYSRFQANPDYQMSGDDT
QHIQQFYDLLTGSMEIIRGWAEKIPGFADLPKADQDLLFESAFLELFVLR
LAYRSNPVEGKLIFCNGVVLHRLQCVRGFGEWIDSIVEFSSNLQNMNIDI
SAFSCIAALAMVTERHGLKEPKRVEELQNKIVNCLKDHVTFNNGGLNRPN
YLSKLLGKLPELRTLCTQGLQRIFYLKLEDLVPPPAIIDKLFLDTLPF.

The term “Pituitary homeobox 3” or “Pitx3” refers to the gene that encodes a member of the RIEG/PITX homeobox family, which is in the bicoid class of homeodomain proteins and act as transcription factors. Pitx3 is involved in the maintenance of dopaminergic neurons. The term “Pitx3” may refer to the nucleotide sequence or protein sequence of human Pitx3 (e.g., Entrez 5309, Uniprot O75364, RefSeq NM_005029.3, or RefSeq NP_005020.1). In embodiments, Pitx3 has the following amino acid sequence:

(SEQ ID NO: 2)
MEFGLLSEAEARSPALSLSDAGTPHPQLPEHGCKGQEHSDSEKASASLPG
GSPEDGSLKKKQRRQRTHFTSQQLQELEATFQRNRYPDMSTREEIAVWTN
LTEARVRVWFKNRRAKWRKRERSQQAELCKGSFAAPLGGLVPPYEEVYPG
YSYGNWPPKALAPPLAAKTFPFAFNSVNVGPLASQPVFSPPSSIAASMVP
SAAAAPGTVPGPGALQGLGGGPPGLAPAAVSSGAVSCPYASAAAAAAAAA
SSPYVYRDPCNSSLASLRLKAKQHASFSYPAVHGPPPAANLSPCQYAVER
PV.

The term “Tyrosine hydroxylase” or “Tyrosine 3-monooxygenase” refers to the enzyme responsible for catalyzing the conversion of the amino acid L-tyosine to L-3,4-dihydroxyphenylalanine (L-DOPA). In humans, tyrosine hydroxylase is encoded by the TH gene. The term “TH” may refer to the nucleotide sequence or protein sequence of human TH (e.g., Entrez 7054, Uniprot P07101, RefSeq NM_199292.2, or RefSeq NP_954986.2). In embodiments, TH has the following amino acid sequence:

(SEQ ID NO: 3)
MPTPDATTPQAKGFRRAVSELDAKQAEAIMVRGQGAPGPSLTGSPWPGTA
APAASYTPTPRSPRFIGRRQSLIEDARKEREAAVAAAAAAVPSEPGDPLE
AVAFEEKEGKAVLNLLFSPRATKPSALSRAVKVFETFEAKIHHLETRPAQ
RPRAGGPHLEYFVRLEVRRGDLAALLSGVRQVSEDVRSPAGPKVPWFPRK
VSELDKCHHLVTKFDPDLDLDHPGFSDQVYRQRRKLIAEIAFQYRHGDPI
PRVEYTAEEIATWKEVYTTLKGLYATHACGEHLEAFALLERFSGYREDNI
PQLEDVSRFLKERTGFQLRPVAGLLSARDFLASLAFRVFQCTQYIRHASS
PMHSPEPDCCHELLGHVPMLADRTFAQFSQDIGLASLGASDEEIEKLSTL
YWFTVEFGLCKQNGEVKAYGAGLLSSYGELLHCLSEEPEIRAFDPEAAAV
QPYQDQTYQSVYFVSESFSDAKDKLRSYASRIQRPFSVKFDPYTLAIDVL
DSPQAVRRSLEGVQDELDTLAHALSAIG.

The term “Vesicular monoamine transporter 2” or “VMAT2” refers to the integral membrane protein that transports neurotransmitters such as dopamine, norepinephrine, serotonin, and histamine, from cellular cytosol into synaptic vesicles. The term “VMAT2” may refer to the nucleotide sequence or protein sequence of human VMAT2 (e.g., Entrez 6571, Uniprot Q05940, RefSeq NM_003054.4, or RefSeq NP_003045.2). In embodiments, VMAT2 has the following amino acid sequence:

(SEQ ID NO: 4)
MALSELALVRWLQESRRSRKLILFIVFLALLLDNMLLTVVVPIIPSYLYS
IKHEKNATEIQTARPVHTASISDSFQSIFSYYDNSTMVTGNATRDLTLHQ
TATQHMVTNASAVPSDCPSEDKDLLNENVQVGLLFASKATVQLITNPFIG
LLTNRIGYPIPIFAGFCIMFVSTIMFAFSSSYAFLLIARSLQGIGSSCSS
VAGMGMLASVYTDDEERGNVMGIALGGLAMGVLVGPPFGSVLYEFVGKTA
PFLVLAALVLLDGAIQLFVLQPSRVQPESQKGTPLTTLLKDPYILIAAGS
ICFANMGIAMLEPALPIWMMETMCSRKWQLGVAFLPASISYLIGTNIFGI
LAHKMGRWLCALLGMIIVGVSILCIPFAKNIYGLIAPNFGVGFAIGMVDS
SMMPIMGYLVDLRHVSVYGSVYAIADVAFCMGYAIGPSAGGAIAKAIGFP
WLMTIIGIIDILFAPLCFFLRSPPAKEEKMAILMDHNCPIKTKMYTQNNI
QSYPIGEDEESESD.

II. Compounds

In an aspect is provided a compound having the formula

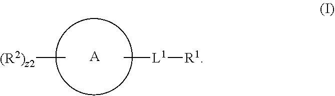

Ring A is aryl or heteroaryl.

L¹ is L¹⁰¹-L¹⁰²-L¹⁰³.

L¹⁰¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene L¹⁰⁴-L¹⁰⁵, L¹⁰⁴-NH-L¹⁰⁵, or L¹⁰⁴-CH₂-L¹⁰⁵.

L¹⁰² is a bond, —S(O)₂—, —N(R¹⁰²), —O—, —S—, —C(O)—, —C(O)N(R¹⁰²)—, —N(_{R}¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹⁰⁴is a bond, —O—, —NH—, —S—, —S(O)₂—, —C(O)—, —NHC(O)—, —C(O)NH—, —OC(O)—, —C(O)O—, substituted or unsubstituted alkylene, or substituted or unsubstituted heteroalkylene.

L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene.

R¹⁰¹, R¹⁰², and R¹⁰³ are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is hydrogen, halogen, —CX CT¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SSR^{1D}, SiR^{1A}R^{1B}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

E is an electrophilic moiety.

R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C (O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituded or unbusbstitued alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl.

n1 and n2 are independently an integer from 0 to 4.

m1, m2, v1, and v2 are independently 1 or 2.

X¹ and X² are independently —F, —Cl, —Br, or —I.

z2 is an integer from 0 to 5.

In embodiments, L¹⁰¹ is a bond, —S(O)₂—, —N(R¹⁰¹)—, —O—, —S—, —C(O)—, —C(O)N(R¹⁰¹)—, —N(_{R}¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene (e.g., C₁-C₈, C₁-C₆, or C₁-C₄), substituted or unsubstituted heteroalkylene (e.g., 2 to 10 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀ or phenylene), substituted or unsubstituted, heteroarylene (e.g., 5 to 10 membered, 5 to 9 membered, or 5 to 6 membered), L¹⁰⁴-L¹⁰⁵, L¹⁰⁴-NH-L¹⁰⁵, or L¹⁰⁴-CH₂-L¹⁰⁵.

In embodiments, L¹⁰¹ is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, R¹⁰¹-substituted or unsubstituted alkylene (e.g., C₁-C₈, C₁-C₆, or C₁-C₄), R¹⁰¹-substituted or unsubstituted heteroalkylene (e.g., 2 to 10 membered, 2 to 6 membered, or 2 to 4 membered), R¹⁰¹-substituted or unsubstituted cycloalkylene (e.g., C₃-C₈, C₃-C₆, or C₅-C₆), R¹⁰¹-substituted or unsubstituted heterocycloalkylene (e.g., 3 to 8 membered, 3 to 6 membered, or 5 to 6 membered), R¹⁰¹-substituted or unsubstituted arylene (e.g., C₆-C₁₀ or phenylene), R¹⁰¹-substituted or unsubstituted heteroarylene (e.g., 5 to 10 membered, 5 to 9 membered, or 5 to 6 membered) _{L}¹⁰⁴-L¹⁰⁵, L¹⁰⁴-NH-L¹⁰⁵or L¹⁰⁴-CH₂-L¹⁰⁵.

In embodiments, L¹⁰² is a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene (e.g., C₁-C₈, C₁-C₆, or C₁-C₄), substituted or unsubstituted heteroalkylene (e.g., 2 to 10 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰² is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, R¹⁰²-substituted or unsubstituted alkylene (e.g., C₁-C₈, C₁-C₆, or C₁-C₄), R¹⁰²-substituted or unsubstituted heteroalkylene (e.g., 2 to 10 membered, 2 to 6 membered, or 2 to 4 membered), R¹⁰²-substituted or unsubstituted cycloalkylene (e.g., C₃-C₈, C₃-C₆, or C₅-C₆), R¹⁰²-substituted or unsubstituted heterocycloalkylene (e.g., 3 to 8 membered, 3 to 6 membered, or 5 to 6 membered), R¹⁰²-substituted or unsubstituted arylene (e.g., C₆-C₁₀ or phenylene), or R¹⁰²-substituted or unsubstituted heteroarylene (e.g., 5 to 10 membered, 5 to 9 membered, or 5 to 6 membered).

In embodiments, L¹⁰³ is a bond, —S(O)₂—, —N(¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene (e.g., C₁-C₈, C₁-C₆, or C₁-C₄), substituted or unsubstituted heteroalkylene (e.g., 2 to 10 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰³ is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, R¹⁰³-substituted or unsubstituted alkylene (e.g., C₁-C₈, C₁-C₆, or C₁-C₄), R¹⁰³-substituted or unsubstituted heteroalkylene (e.g., 2 to 10 membered, 2 to 6 membered, or 2 to 4 membered), R¹⁰³-substituted or unsubstituted cycloalkylene (e.g., C₃-C₈, C₃-C₆, or C₅-C₆), R¹⁰³-substituted or unsubstituted heterocycloalkylene (e.g., 3 to 8 membered, 3 to 6 membered, or 5 to 6 membered), R¹⁰³-substituted or unsubstituted arylene (e.g., C₆-C₁₀ or phenylene), or R¹⁰³-substituted or unsubstituted heteroarylene (e.g., 5 to 10 membered, 5 to 9 membered, or 5 to 6 membered).

In embodiments, R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, or C₁-C₄), substituted or unsubstituted heteroalkyl (e.g., 2 to 10 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

In embodiments, R^{1A}, R^{1B}, R^{1C}, and R^{ig} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹⁰-substituted or unsubstituted alkyl (e.g., C₁-C₈, C₁-C₆, or C₁-C₄), R¹⁰-substituted or unsubstituted heteroalkyl (e.g., 2 to 10 membered, 2 to 6 membered, or 2 to 4 membered), R¹⁰-substituted or unsubstituted cycloalkyl (e.g., C₃-C₈, C₃-C₆, or C₅-C₆), R¹⁰-substituted or unsubstituted heterocycloalkyl (e.g., 3 to 8 membered, 3 to 6 membered, or 5 to 6 membered), R¹⁰-substituted or unsubstituted aryl (e.g., C₆-C₁₀ or phenyl), or R¹⁰-substituted or unsubstituted heteroaryl (e.g., 5 to 10 membered, 5 to 9 membered, or 5 to 6 membered); R^{1A} and R^{1B} substituents bonded to the same nitrogen atom may be joined to form an R¹⁰-substituted or unsubstituted heterocycloalkyl (e.g., 3 to 8 membered, 3 to 6 membered, or 5 to 6 membered) or R¹⁰-substituted or unsubstituted heteroaryl (e.g., 5 to 10 membered, 5 to 9 membered, or 5 to 6 membered).

In embodiments, R^{2A}, R^{2B}, R^{2C}, and R^{2D} are independently hydrogen, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, substituted or unsubstituted alkyl (e.g., C₁-C₈, C₁-C₆, or C₁-C₄), substituted or unsubstituted heteroalkyl (e.g., 2 to 10 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

In embodiments, R^{2A}, R^{2B}, R^{2C}, and R^{2D} are independently hydrogen, halogen, —CCl₃, —CF₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, R²⁰-substituted or unsubstituted alkyl (e.g., C₁-C₈, C₁-C₆, or C₁-C₄), R²⁰-substituted or unsubstituted heteroalkyl (e.g., 2 to 10 membered, 2 to 6 membered, or 2 to 4 membered), R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered); R^{2A} and R^{2B} substituents bonded to the same nitrogen atom may be joined to form an R²⁰-substituted or unsubstituted heterocycloalkyl (e.g., 3 to 8 membered, 3 to 6 membered, or 5 to 6 membered) or R²⁰-substituted or unsubstituted heteroaryl (e.g., 5 to 10 membered, 5 to 9 membered, or 5 to 6 membered).

In embodiments, -L¹-R¹ is

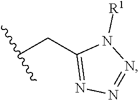

and R¹ is as described herein, including in embodiments.

In embodiments, the compound has the formula

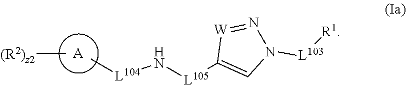

Ring A, R¹, R², L¹⁰³, L¹⁰⁴, L¹⁰⁵, and z2 are as described herein.

W is N or CH.

In embodiments, L¹⁰³ is a bond, substituted or unsubstituted alkylene, or substituted or unsubstituted heteroalkylene.

In embodiments, L¹⁰⁴ is a bond, —S(O)₂—, —C(O)—, —NHC(O)—, —OC(O)—, substituted or unsubstituted alkylene, or substituted or unsubstituted heteroalkylene.

In embodiments, L¹⁰⁵ is a bond, substituted or unsubstituted alkylene, substituted or unsubstituted heteroalkylene, substituted or unsubstituted cycloalkylene, or substituted or unsubstituted heterocycloalkylene.

In embodiments, Ring A is aryl (e.g., C₆-C₁₀ or phenyl) or heteroaryl (e.g., 5 to 10 membered, 5 to 9 membered, or 5 to 6 membered). In embodiments, Ring A is a C₆-C₁₀ aryl. In embodiments, Ring A is a phenyl. In embodiments, Ring A is a 5 to 10 membered heteroaryl. In embodiments, Ring A is a 5 to 9 membered heteroaryl. In embodiments, Ring A is a 5 to 6 membered heteroaryl.

In embodiments, Ring A is a phenyl or 5 to 10 membered heteroaryl. In embodiments, Ring A is a phenyl. In embodiments, Ring A is a naphthyl. In embodiments, Ring A is a quinolinyl. In embodiments, Ring A is an isoquinolinyl. In embodiments, Ring A is

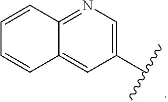 [SMILES: *c1cnc2ccccc2c1]

In embodiments, Ring A is a phenyl or 5 to 10 membered heteroaryl. In embodiments, Ring A is a phenyl. In embodiments, Ring A is a naphthyl. In embodiments, Ring A is a quinolinyl. In embodiments, Ring A is an isoquinolinyl. In embodiments, Ring A is a benzoxazolyl. In embodiments, Ring A is

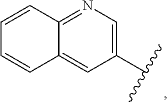

wherein [symbol] denotes attachment point to -L¹-R¹. In embodiments, Ring A is

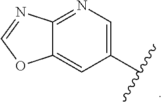

wherein [symbol] denotes the attachment point to -L¹-R¹.

In embodiments, Ring A is a phenyl or 5 to 10 membered heteroaryl. In embodiments, Ring A is a phenyl. In embodiments, Ring A is a naphthyl. In embodiments, Ring A is a quinolinyl. In embodiments, Ring A is a 3-quinolinyl. In embodiments, Ring A is an isoquinolinyl. In embodiments, Ring A is a benzoxazolyl. In embodiments, Ring A is a 6-benzoxazolyl.

In embodiments, the compound has the formula

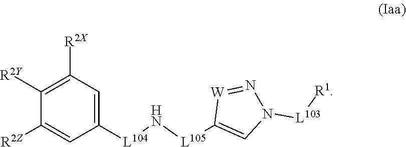

R¹, L¹⁰³, L¹⁰⁴, L¹⁰⁵, and W are as described herein.

R^{2X}, R^{2Y}, and R^{2Z} are independently hydrogen, or may independently assume any value of R², including in embodiments.

In embodiments, R^{2X}, R^{2Y}, and R^{2Z} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)^{m2}, —NR^{2A}R^{2B}, —C(O)R^{2C}, —C(O)OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C},—N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2X} and R^{2Y} substituents may be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2Y} and R^{2Z} substituents may be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{2X}, R^{2Y}, and R^{2Z} are independently hydrogen, halogen, —CX²₃, —CHX²₂, —CH₂X², —OCX²₃, —OCH₂X², —OCHX²₂, —CN, —SO₂R^{2D}, —SONR^{2A}R^{2B}, —NHC(O)NR^{2A}R^{2B}, —N(O)₂, —NR^{2A}R^{2B}, —C(O)R^{2C}, —C(O)OR^{2C}, —C(O)NR^{2A}R^{2B}, —OR^{2D}, —NR^{2A}SO₂R^{D2}, —NR^{2A}C(O)R^{2C}, NR^{2A}OR^{2C}, —N^{2A}OR^{2C}, —N₃, R²⁰-substituted or unsubstituted alkyl (e.g., C₁-C₈, C₁-C₆, or C₁-C₄), R²⁰-substituted or unsubstituted heteroalkyl (e.g., 2 to 8 membered, 2 to 6 membered, or 2 to 4 membered), R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered); R^{2X} and R^{2Y} substituents may be joined to form an R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered); R^{2Y} and R^{2Z} substituents may be joined to form an R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered).

In embodiments, R^{2X} is independently halogen or unsubstituted heteroalkyl; R^{2Y} is independently hydrogen or halogen; and R^{2Z} is independently hydrogen, halogen, —CN, —NR^{2A}C(O)R^{2C}, unsubstituted heteroalkyl, or substituted or unsubstituted heterocycloalkyl.

In embodiments, R^{2X} is independently halogen or unsubstituted heteroalkyl. In embodiments, R^{2Y} is independently hydrogen or halogen. In embodiments, R^{2Z} is independently hydrogen, halogen, —CN, —NR^{2A}C(O)R^{2C}, unsubstitued heteroalkyl, or substituted or unsubstituted heterocycloalkyl. In embodiments, R^{2X} is independently halogen. In embodiments, R^{2X} is independently unsubstituted heteroalkyl. In embodiments, R^{2Y} is independently hydrogen. In embodiments, R^{2Y} is independently halogen. In embodiments, R^{2Z} is independently hydrogen. In embodiments, R^{2Z} is independently halogen. In embodiments, R^{2Z} is independently —CN. In embodiments, R^{2Z} is independently —NR^{2A}C(O)R^{2C}. In embodiments, R^{2Z} is independently unsubstitued heteroalkyl. In embodiments, R^{2Z} is independently substituted or unsubstituted heterocycloalkyl.

In embodiments, R^{2X} is independently halogen; R^{2Y} is independently halogen; and R^{2Z} is independently hydrogen.

In embodiments, R^{2X} is independently halogen. In embodiments, R^{2Y} is independently halogen. In embodiments, R^{2Z} is independently hydrogen.

In embodiments, R^{2X} is independently halogen or unsubstituted 2 to 4 membered heteroalkyl; R^{2Y} is independently hydrogen; R^{2Z} is independently halogen, —CN, —NR^{2A}C(O)R^{2C}, unsubstituted 2 to 4 membered heteroalkyl, or substituted or unsubstituted 5 to 6 membered heterocycloalkyl; R^{2A} is independently hydrogen; and R^{2C} is independently unsubstituted C₁-C₂ alkyl.

In embodiments, R^{2X} is independently halogen or unsubstituted 2 to 4 membered heteroalkyl. In embodiments, R^{2Y} is independently hydrogen. In embodiments, R^{2Z} is independently halogen, —CN, —NR^{2A}C(O)R^{2C}, unsubstituted 2 to 4 membered heteroalkyl, or substituted or unsubstituted 5 to 6 membered heterocycloalkyl. In embodiments, R^{2A} is independently hydrogen. In embodiments, R^{2C} is independently unsubstituted C₁-C₂ alkyl.

In embodiments, R^{2X} is independently halogen. In embodiments, R^{2X} is independently unsubstituted 2 to 4 membered heteroalkyl. In embodiments, R^{2Z} is independently halogen. In embodiments, R^{2Z} is independently —CN. In embodiments, R^{2Z} is independently —NR^{2A}C(O)R^{2C}. In embodiments R^{2Z} independently unsubstituted 2 to 4 membered heteroalkyl. In embodiments, R^{2Z} is independently substituted or unsubstituted 5 to 6 membered heterocycloalkyl.

In embodiments, R^{2X} is independently halogen or —OCH₃; R^{2Y} is independently hydrogen; R^{2Z} is independently halogen, —CN, —NHC(O)CH₃, —OCH₃, or substituted or unsubstituted 5 to 6 membered heterocycloalkyl; R^{2A} is independently hydrogen; and R^{2C} is independently unsubstituted C₁-C₂ alkyl.

In embodiments, R^{2X} is independently halogen or —OCH₃. In embodiments, R^{2Y} is independently hydrogen. In embodiments, R^{2Z} is independently halogen, —CN, —NHC(O)CH₃, —OCH₃, or substituted or unsubstituted 5 to 6 membered heterocycloalkyl. In embodiments, R^{2A} is independently hydrogen. In embodiments, R^{2C} is independently unsubstituted C₁-C₂ alkyl. In embodiments, R^{2X} is independently halogen. In embodiments, R^{2X} is independently —OCH₃. In embodiments, R^{2Z} is independently halogen. In embodiments, R^{2Z} is independently —CN. In embodiments, R^{2Z} is independently —NHC(O)CH₃. In embodiments, R^{2Z} is independently —OCH₃. In embodiments, R^{2Z} is independently substituted or unsubstituted 5 to 6 membered heterocycloalkyl.

In embodiments, R^{2X} is independently halogen or —OCH₃; R^{2Y} is independently hydrogen; R^{2Z} is independently halogen, —CN, —NHC(O)CH₃, —OCH₃, or substituted or unsubstituted 5 to 6 membered heterocycloalkyl. In embodiments, R^{2Z} is independently substituted or unsubstituted 5 to 6 membered heterocycloalkyl. In embodiments, R^{2Z} is independently substituted 5 to 6 membered heterocycloalkyl. In embodiments, R^{2Z} is independently

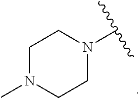 [SMILES: *N1CCN(C)CC1]

In embodiments, L¹⁰³ is a bond, substituted or unsubstituted alkylene (e.g., C₁-C₈, C₁-C₆, or C₁-C₄), or substituted or unsubstituted heteroalkylene (e.g., 2 to 8 membered, 2 to 6 membered, or 2 to 4 membered). In embodiments, L¹⁰³ is a bond. In embodiments, L¹⁰³ is substituted or unsubstituted C₁-C₈ alkylene. In embodiments, L¹⁰³ is substituted or unsubstituted C₁-C₆ alkylene. In embodiments, L¹⁰³ is substituted or unsubstituted C₁-C₄ alkylene. In embodiments, L¹⁰³ is substituted or unsubstituted 2 to 8 membered heteroalkylene. In embodiments, L¹⁰³ is substituted or unsubstituted 2 to 6 membered heteroalkylene. In embodiments, L¹⁰³ is substituted or unsubstituted 2 to 4 membered heteroalkylene.

In embodiments, L¹⁰³ is an unsubstituted alkylene. In embodiments, L¹⁰³ is an unsubstituted C₁-C₄ alkylene. In embodiments, L¹⁰³ is an unsubstituted ethylene.

In embodiments, L¹⁰³ is a bond, R¹⁰³-substituted or unsubstituted alkylene (e.g., C₁-C₈, C₁-C₆, or C₁-C₄), or R¹⁰³-substituted or unsubstituted heteroalkylene (e.g., 2 to 8 membered, 2 to 6 membered, or 2 to 4 membered).

In embodiments, R¹⁰¹, R¹⁰², and R¹⁰³ are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰¹, R¹⁰², and R¹⁰³ are independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰¹ is independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R^{ill}-substituted or unsubstituted alkyl (e.g., C₁-C₈, C₁-C₆, C₁-C₄, or C₁-C₂), R¹¹¹-substituted or unsubstituted heteroalkyl (e.g., 2 to 8 membered, 2 to 6 membered, 4 to 6 membered, 2 to 3 membered, or 4 to 5 membered), R¹¹¹-substituted or unsubstituted cycloalkyl (e.g., C₃-C₈, C₃-C₆, C₄-C₆, or C₅-C₆), R″-substituted or unsubstituted heterocycloalkyl (e.g., 3 to 8 membered, 3 to 6 membered, 4 to 6 membered, 4 to 5 membered, or 5 to 6 membered), R^{ill}-substituted or unsubstituted aryl (e.g., C₆-C₁₀ or phenyl), or R^{ill}-substituted or unsubstituted heteroaryl (e.g., 5 to 10 membered, 5 to 9 membered, or 5 to 6 membered).

In embodiments, R¹⁰¹ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R″-substituted or unsubstituted alkyl (e.g., C₁-C₈, C₁-C₆, C₁-C₄, or C₁-C₂), R¹¹¹-substituted or unsubstituted heteroalkyl (e.g., 2 to 8 membered, 2 to 6 membered, 4 to 6 membered, 2 to 3 membered, or 4 to 5 membered), R″-substituted or unsubstituted cycloalkyl (e.g., C₃-C₈, C₃-C₆, C₄-C₆, or C₅-C₆), R″-substituted or unsubstituted heterocycloalkyl (e.g., 3 to 8 membered, 3 to 6 membered, 4 to 6 membered, 4 to 5 membered, or 5 to 6 membered), R^{ill}-substituted or unsubstituted aryl (e.g., C₆-C₁₀ or phenyl), or R¹¹¹-substituted or unsubstituted heteroaryl (e.g., 5 to 10 membered, 5 to 9 membered, or 5 to 6 membered).

R¹¹¹ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁰¹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, W° ² is independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹¹²-substituted or unsubstituted alkyl (e.g., C₁-C₈, C₁-C₆, C₁-C₄, or C₁-C₂), R¹¹²-substituted or unsubstituted heteroalkyl (e.g., 2 to 8 membered, 2 to 6 membered, 4 to 6 membered, 2 to 3 membered, or 4 to 5 membered), R¹¹²-substituted or unsubstituted cycloalkyl (e.g., C₃-C₈, C₃-C₆, C₄-C₆, or C₅-C₆), R¹¹²-substituted or unsubstituted heterocycloalkyl (e.g., 3 to 8 membered, 3 to 6 membered, 4 to 6 membered, 4 to 5 membered, or 5 to 6 membered), R¹¹²-substituted or unsubstituted aryl (e.g., C₆-C₁₀ or phenyl), or R¹¹²-substituted or unsubstituted heteroaryl (e.g., 5 to 10 membered, 5 to 9 membered, or 5 to 6 membered).

In embodiments, W° ² is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹¹²-substituted or unsubstituted alkyl (e.g., C₁-C₈, C₁-C₆, C₁-C₄, or C₁-C₂), R¹¹²-substituted or unsubstituted heteroalkyl (e.g., 2 to 8 membered, 2 to 6 membered, 4 to 6 membered, 2 to 3 membered, or 4 to 5 membered), R¹¹²-substituted or unsubstituted cycloalkyl (e.g., C₃-C₈, C₃-C₆, C₄-C₆, or C₅-C₆), R¹¹²-substituted or unsubstituted heterocycloalkyl (e.g., 3 to 8 membered, 3 to 6 membered, 4 to 6 membered, 4 to 5 membered, or 5 to 6 membered), R¹¹²-substituted or unsubstituted aryl (e.g., C₆-C₁₀ or phenyl), or R¹¹²-substituted or unsubstituted heteroaryl (e.g., 5 to 10 membered, 5 to 9 membered, or 5 to 6 membered).

R¹¹² is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁰² is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R^{1B} is independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —CO OH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R^{1B}-substituted or unsubstituted alkyl (e.g., C₁-C₈, C₁-C₆, or C₁-C₄), R^{1B}-substituted or unsubstituted heteroalkyl (e.g., 2 to 8 membered, 2 to 6 membered, or 2 to 4 membered), R^{1B}-substituted or unsubstituted cycloalkyl (e.g., C₃-C₈, C₃-C₆, or C₅-C₆), R^{1B}-substituted or unsubstituted heterocycloalkyl (e.g., 3 to 8 membered, 3 to 6 membered, or 5 to 6 membered), R^{1B}-substituted or unsubstituted aryl (e.g., C₆-C₁₀ or phenyl), or R^{1B}-substituted or unsubstituted heteroaryl (e.g., 5 to 10 membered, 5 to 9 membered, or 5 to 6 membered).

In embodiments, R¹⁰³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R^{1B}-substituted or unsubstituted alkyl (e.g., C₁-C₈, C₁-C₆, or C₁-C₄), R^{1B}-substituted or unsubstituted heteroalkyl (e.g., 2 to 8 membered, 2 to 6 membered, or 2 to 4 membered), R^{1B}-substituted or unsubstituted cycloalkyl (e.g., C₃-C₈, C₃-C₆, or C₅-C₆), R^{1B}-substituted or unsubstituted heterocycloalkyl (e.g., 3 to 8 membered, 3 to 6 membered, or 5 to 6 membered), R^{1B}-substituted or unsubstituted aryl (e.g., C₆-C₁₀ or phenyl), or R^{1B}-substituted or unsubstituted heteroaryl (e.g., 5 to 10 membered, 5 to 9 membered, or 5 to 6 membered).

R^{1B} is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁰³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, L¹⁰⁴ is a bond, —S(O)₂—, —C(O)—, —NHC(O)—, —C(O)NH—, —OC(O)—, —C(O)O—, substituted or unsubstituted alkylene, or substituted or unsubstituted heteroalkylene.

In embodiments, L¹⁰⁴ is a bond, —O—, —NH—, —S—, —S(O)₂—, —C(O)—, —NHC(O)—, —C(O)NH—, —OC(O)—, —C(O)O—, substituted or unsubstituted alkylene (e.g., C₁-C₈, C₁-C₆, or C₁-C₄), or substituted or unsubstituted heteroalkylene (e.g., 2 to 8 membered, 2 to 6 membered, or 2 to 4 membered). In embodiments, L¹⁰⁴ is a bond. In embodiments, L¹⁰⁴ is —O—. In embodiments, L¹⁰⁴ is —NH—. In embodiments, L¹⁰⁴ is —S—. In embodiments, L¹⁰⁴ is —S(O)₂—. In embodiments, L¹⁰⁴ is —C(O)—.) In embodiments, L¹⁰ is —NHC(O)—. In embodiments, L¹⁰⁴ is —C(O)NH—. In embodiments, L¹⁰⁴ is —OC(O)—. In embodiments, L¹⁰⁴ is —C(O)O—. In embodiments, L¹⁰⁴ is substituted or unsubstituted C₁-C₈ alkylene. In embodiments, L¹⁰⁴ is substituted or unsubstituted C₁-C₆ alkylene. In embodiments, L¹⁰⁴ is substituted or unsubstituted C₁-C₄ alkylene. In embodiments, L¹⁰⁴ is substituted or unsubstituted 2 to 8 membered heteroalkylene. In embodiments, L¹⁰ is substituted or unsubstituted 2 to 6 membered heteroalkylene. In embodiments, L¹⁰ is substituted or unsubstituted 2 to 4 membered heteroalkylene. In embodiments, L¹⁰ is unsubstituted C₁-C₈ alkylene. In embodiments, L¹⁰⁴ is unsubstituted 2 to 8 membered heteroalkylene.

In embodiments, L¹⁰⁴ is a bond, —S(O)₂—, —C(O)—, —NHC(O)—, —OC(O)—, substituted or unsubstituted alkylene (e.g., C₁-C₈, C₁-C₆, or C₁-C₄), or substituted or unsubstituted heteroalkylene (e.g., 2 to 8 membered, 2 to 6 membered, or 2 to 4 membered). In embodiments, L¹⁰⁴ is a bond. In embodiments, L¹⁰⁴ is —S(O)₂—. In embodiments, L¹⁰⁴ is —C(O)—. In embodiments, L¹⁰⁴ is —NHC(O). In embodiments, L¹⁰⁴ is —OC(O)—. In embodiments, L¹⁰⁴ is substituted or unsubstituted C₁-C₈ alkylene. In embodiments, L¹⁰⁴ is substituted or unsubstituted C₁-C₆ alkylene. In embodiments, L¹⁰⁴ is substituted or unsubstituted C₁-C₄ alkylene. In embodiments, L¹⁰ is substituted or unsubstituted 2 to 8 membered heteroalkylene. In embodiments, L¹⁰⁴ is substituted or unsubstituted 2 to 6 membered heteroalkylene. In embodiments, L¹⁰⁴ is substituted or unsubstituted 2 to 4 membered heteroalkylene. In embodiments, L¹⁰⁴ is unsubstituted C₁-C₈ alkylene. In embodiments, L¹⁰⁴ is unsubstituted 2 to 8 membered heteroalkylene.

In embodiments, L¹⁰⁴ is a bond, —O—, —NH—, —S—, —S(O)₂—, —C(O)—, —NHC(O)—, —C(O)NH—, —OC(O)—, —C(O)O—, R¹⁰⁴-substituted or unsubstituted alkylene (e.g., C₁-C₈, C₁-C₆, or C₁-C₄), or R¹⁰⁴-substituted or unsubstituted heteroalkylene (e.g., 2 to 8 membered, 2 to 6 membered, or 2 to 4 membered).

In embodiments, L¹⁰⁴ is a bond, —S(O)₂—, —C(O)—, —NHC(O)—, —OC(O)—, R¹⁰⁴-substituted or unsubstituted alkylene (e.g., C₁-C₈, C₁-C₆, or C₁-C₄), or R¹⁰⁴-substituted or unsubstituted heteroalkylene (e.g., 2 to 8 membered, 2 to 6 membered, or 2 to 4 membered).

R¹⁰⁴ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰⁴ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹¹⁴-substituted or unsubstituted alkyl (e.g., C₁-C₈, C₁-C₆, or C₁-C₄), R¹¹⁴-substituted or unsubstituted heteroalkyl (e.g., 2 to 8 membered, 2 to 6 membered, or 2 to 4 membered), R¹¹⁴-substituted or unsubstituted cycloalkyl (e.g., C₃-C₈, C₃-C₆, or C₅-C₆), R¹¹⁴-substituted or unsubstituted heterocycloalkyl (e.g., 3 to 8 membered, 3 to 6 membered, or 5 to 6 membered), R¹¹⁴-substituted or unsubstituted aryl (e.g., C₆-C₁₀ or phenyl), or R¹¹⁴-substituted or unsubstituted heteroaryl (e.g., 5 to 10 membered, 5 to 9 membered, or 5 to 6 membered).

R¹¹⁴ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), or substituted or unsubstituted heterocycloalkylene (e.g., 3 to 8 membered, 3 to 6 membered, or 5 to 6 membered).

In embodiments, L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), or substituted or unsubstituted heterocycloalkylene (e.g., 3 to 8 membered, 3 to 6 membered, or 5 to 6 membered). In embodiments, L¹⁰⁵ is a bond. In embodiments, L¹⁰⁵ is —O—. In embodiments, L¹⁰⁵ is —NH—. In embodiments, L¹⁰⁵ is —S—. In embodiments, L¹⁰⁵ is —S(O)₂—. In embodiments, L¹⁰⁵ is —C(O)—. In embodiments, L¹⁰⁵ is —NHC(O)—. In embodiments, L¹⁰⁵ is —C(O)NH—. In embodiments, L¹⁰⁵ is —OC(O)—. In embodiments, L¹⁰⁵ is —C(O)O—. In embodiments, L¹⁰⁵ is substituted or unsubstituted C₁-C₈ alkylene. In embodiments, L¹⁰⁵ is substituted or unsubstituted C₁-C₆ alkylene. In embodiments, L¹⁰⁵ is substituted or unsubstituted C₁-C₄ alkylene. In embodiments, L¹⁰⁵ is substituted or unsubstituted 2 to 8 membered hetereoalkylene. In embodiments, L¹⁰⁵ is substituted or unsubstituted 2 to 6 membered heteroalkylene. In embodiments, L¹⁰⁵ is substituted or unsubstituted 2 to 4 membered heteroalkylene. In embodiments, L¹⁰⁵ is substituted or unsubstituted C₃-C₈ cycloalkylene. In embodiments, L¹⁰⁵ is substituted or unsubstituted C₃-C₆ cycloalkylene. In embodiments, L¹⁰⁵ is substituted or unsubstituted C₅-C₆ cycloalkylene. In embodiments, L¹⁰⁵ is substituted or unsubstituted 3 to 8 membered heterocycloalkylene. In embodiments, L¹⁰⁵ is substituted or unsubstituted 3 to 6 membered heterocycloalkylene. In embodiments, L¹⁰⁵ is substituted or unsubstituted 5 to 6 membered heterocycloalkylene. In embodiments, L¹⁰⁵ is unsubstituted C₁-C₈ alkylene. In embodiments, L¹⁰⁵ is unsubstituted 2 to 8 membered hetereoalkylene. In embodiments, L¹⁰⁵ is unsubstituted C₃-C₈ cycloalkylene. In embodiments, L¹⁰⁵ is unsubstituted 3 to 8 membered heterocycloalkylene.

In embodiments, L¹⁰⁵ is a bond, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), or substituted or unsubstituted heterocycloalkylene (e.g., 3 to 8 membered, 3 to 6 membered, or 5 to 6 membered). In embodiments, L¹⁰⁵ is a bond. In embodiments, L¹⁰⁵ is substituted or unsubstituted C₁-C₈ alkylene. In embodiments, L¹⁰⁵ is substituted or unsubstituted C₁-C₆ alkylene. In embodiments, L¹⁰⁵ is substituted or unsubstituted C₁-C₄ alkylene. In embodiments, L¹⁰⁵ is substituted or unsubstituted 2 to 8 membered hetereoalkylene. In embodiments, L¹⁰⁵ is substituted or unsubstituted 2 to 6 membered heteroalkylene. In embodiments, L¹⁰⁵ is substituted or unsubstituted 2 to 4 membered heteroalkylene. In embodiments, L¹⁰⁵ is substituted or unsubstituted C₃-C₈ cycloalkylene. In embodiments, L¹⁰⁵ is substituted or unsubstituted C₃-C₆ cycloalkylene. In embodiments, L¹⁰⁵ is substituted or unsubstituted C₅-C₆ cycloalkylene. In embodiments, L¹⁰⁵ is substituted or unsubstituted 3 to 8 membered heterocycloalkylene. In embodiments, L¹⁰⁵ is substituted or unsubstituted 3 to 6 membered heterocycloalkylene. In embodiments, L¹⁰⁵ is substituted or unsubstituted 5 to 6 membered heterocycloalkylene. In embodiments, L¹⁰⁵ is unsubstituted C₁-C₈ alkylene. In embodiments, L¹⁰⁵ is unsubstituted 2 to 8 membered hetereoalkylene. In embodiments, L¹⁰⁵ is unsubstituted C₃-C₈ cycloalkylene. In embodiments, L¹⁰⁵ is unsubstituted 3 to 8 membered heterocycloalkylene.

In embodiments, L¹⁰⁵ is an unsubstituted alkylene. In embodiments, L¹⁰⁵ is an unsubstituted C₁-C₄ alkylene. In embodiments, L¹⁰⁵ is

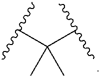 [SMILES: *C(*)(C)C]

In embodiments, L¹⁰⁵ is

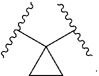 [SMILES: *C1(*)CC1]

In embodiments, L¹⁰⁵ is a bond, —O—, —NH—, —S—, —S(O)₂—, —C(O)—, —NHC(O)—, —C(O)NH—, —OC(O)—, —C(O)O—, R¹⁰⁵-substituted or unsubstituted alkylene (e.g., C₁-C₈, C₁-C₆, or C₁-C₄), R¹⁰⁵-substituted or unsubstituted heteroalkylene (e.g., 2 to 8 membered, 2 to 6 membered, or 2 to 4 membered), R¹⁰⁵-substituted or unsubstituted cycloalkylene (e.g., C₃-C₈, C₃-C₆, or C₅-C₆), or R¹⁰⁵-substituted or unsubstituted heterocycloalkylene (e.g., 3 to 8 membered, 3 to 6 membered, or 5 to 6 membered).

In embodiments, L^{11/5} is a bond, R¹⁰⁵-substituted or unsubstituted alkylene (e.g., C₁-C₈, C₁-C₆, or C₁-C₄), R¹⁰⁵-substituted or unsubstituted heteroalkylene (e.g., 2 to 8 membered, 2 to 6 membered, or 2 to 4 membered), R¹⁰⁵-substituted or unsubstituted cycloalkylene (e.g., C₃-C₈, C₃-C₆, or C₅-C₆), or R¹⁰⁵-substituted or unsubstituted heterocycloalkylene (e.g., 3 to 8 membered, 3 to 6 membered, or 5 to 6 membered).

R¹⁰⁵ is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{10s} is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, R¹¹⁵-substituted or unsubstituted alkyl (e.g., C₁-C₈, C₁-C₆, or C₁-C₄), R¹¹⁵-substituted or unsubstituted heteroalkyl (e.g., 2 to 8 membered, 2 to 6 membered, or 2 to 4 membered), R¹¹⁵-substituted or unsubstituted cycloalkyl (e.g., C₃-C₈, C₃-C₆, or C₅-C₆), R¹¹⁵-substituted or unsubstituted heterocycloalkyl (e.g., 3 to 8 membered, 3 to 6 membered, or 5 to 6 membered), R¹¹⁵-substituted or unsubstituted aryl (e.g., C₆-C₁₀ or phenyl), or R¹¹⁵-substituted or unsubstituted heteroaryl (e.g., 5 to 10 membered, 5 to 9 membered, or 5 to 6 membered).

R¹¹⁵ is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, W is N. In embodiments, W is CH.

In embodiments,

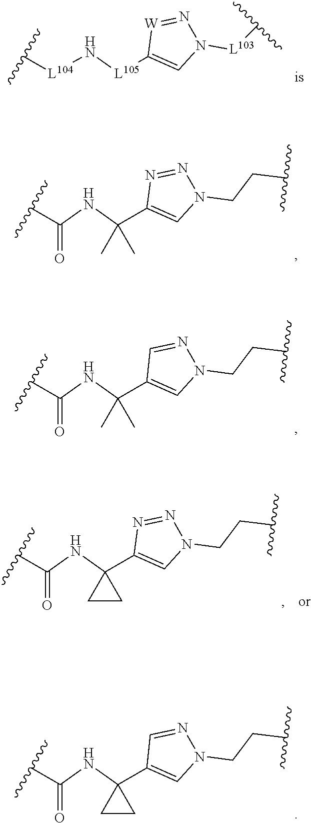

In embodiments,

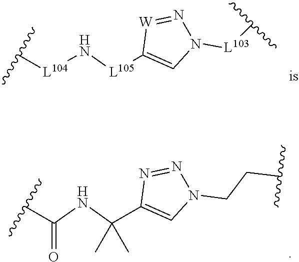

In embodiments,

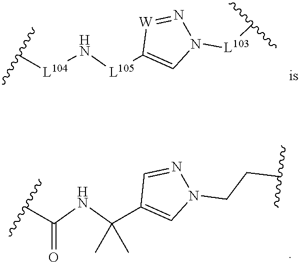

In embodiments,

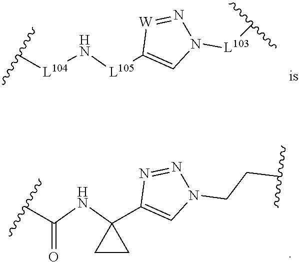

In embodiments,

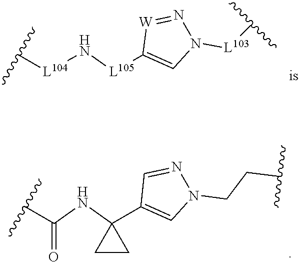

In embodiments,

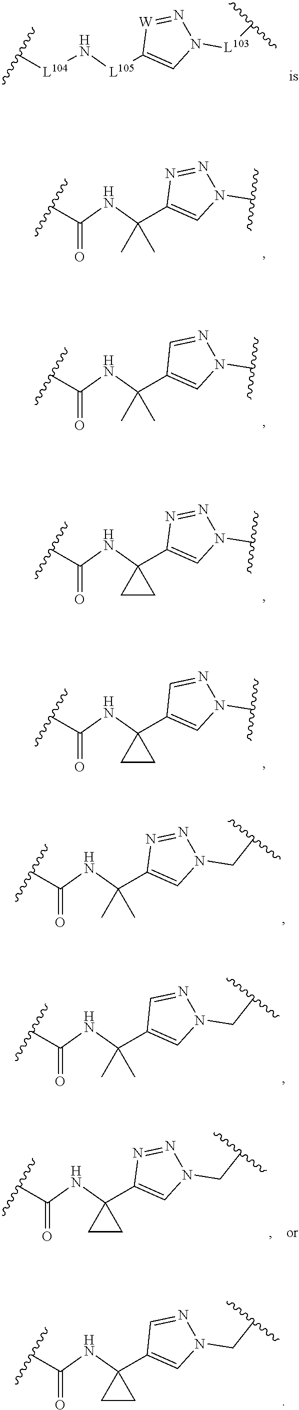

In embodiments,

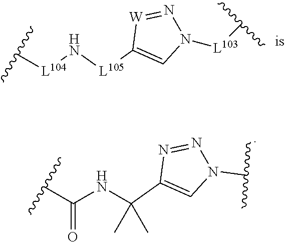

In embodiments,

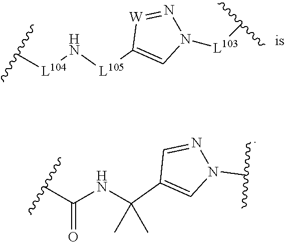

In embodiments,

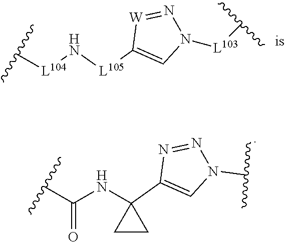

In embodiments,

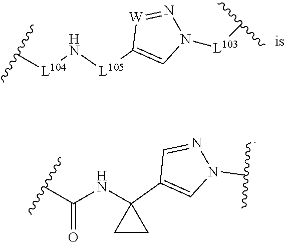

In embodiments,

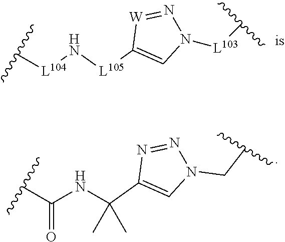

In embodiments,

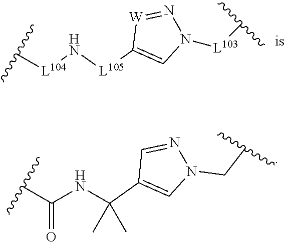

In embodiments,

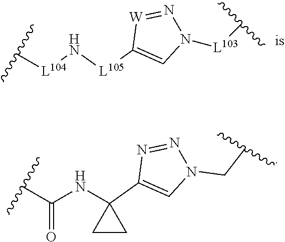

In embodiments,

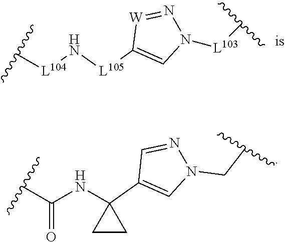

In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)_{R}^{1C}, —NR^{1A}C(O)OR^{1C}, —NR^{1A}OR^{1C}, —N₃, —SSR^{1D}, —SiR^{1A}R^{1B}R^{1C}, E, substituted or unsubstituted alkyl, substituted or umsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is hydrogen, halogen, —CX¹₃, —CHX¹₂, —CH₂X¹, —OCX¹₃, —OCH₂X¹, —OCHX¹₂, —CN, —SO₁NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C} —NR^{1A}OR^{1C}, —N₃, —SiR^{1A}R^{1B}R^{1C}, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{2B}, —N(O)ₘ₁, —OCX¹₃, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SiR^{1A}R^{1B}R^{1C}, —SP(O)(OH)₂, E, substituted or unsubstituted alkyl (e.g., C₁-C₈, C₁-C₆, or C₁-C₄), substituted or unsubstituted heteroalkyl (e.g., 2 to 10 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}, R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SiR^{1A}R^{1B}R^{1C}, —SP(O)(OH)₂, E, R¹⁰-substituted or unsubstituted alkyl (e.g., C₁-C₈, C₁-C₆, or C₁-C₄), R^{w}-substituted or unsubstituted heteroalkyl (e.g., 2 to 10 membered, 2 to 6 membered, or 2 to 4 membered), R¹⁰-substituted or unsubstituted cycloalkyl (e.g., C₃-C₈, C₃-C₆, or C₅-C₆), R¹⁰-substituted or unsubstituted heterocycloalkyl (e.g., 3 to 8 membered, 3 to 6 membered, or 5 to 6 membered), R¹⁰-substituted or unsubstituted aryl (e.g., C₆-C₁₀ or phenyl), or R¹⁰-substituted or unsubstituted heteroaryl (e.g., 5 to 10 membered, 5 to 9 membered, or 5 to 6 membered).

In embodiments, R¹ is independently —C(O)R^{1C}.

In embodiments, R¹ is independently —SC(O)R^{1C}.

In embodiments, R^{1C} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, substituted or unsubstituted alkyl (e.g., C₁-C₈, C₁-C₆, or C₁-C₄), substituted or unsubstituted heteroalkyl (e.g., 2 to 10 membered, 2 to 6 membered, or 2 to 4 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{1C} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, -01\1112, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted alkyl (e.g., C₁-C₈, C₁-C₆, or C₁-C₄), R¹⁰-substituted or unsubstituted heteroalkyl (e.g., 2 to 10 membered, 2 to 6 membered, or 2 to 4 membered), R¹⁰-substituted or unsubstituted aryl (e.g., C₆-C₁₀ or phenyl), or R¹⁰-substituted or unsubstituted heteroaryl (e.g., 5 to 10 membered, 5 to 9 membered, or 5 to 6 membered).

In embodiments, R^{1C} is independently substituted or unsubstituted C₁-C₄ alkyl. In embodiments, R^{1C} is independently unsubstituted C₁-C₄ alkyl. In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted n-propyl. In embodiments, R^{1C} is independently unsubstituted isopropyl. In embodiments, R^{1C} is independently unsubstituted butyl. In embodiments, R^{1C} is independently unsubstituted n-butyl. In embodiments, R^{1C} is independently unsubstituted tert-butyl.

In embodiments, R^{1C} is independently substituted or unsubstituted aryl. In embodiments, R^{1C} is independently R¹⁰-substituted or unsubstituted aryl. In embodiments, R^{1C} is independently R¹⁰-substituted or unsubstituted phenyl. In embodiments, R^{1C} is independently unsubstituted phenyl.

In embodiments, R¹ is independently —C(O)R^{1C}, and R^{1C} is as described herein, including in embodiments. In embodiments, R¹ is independently —C(O)OH. In embodiments, R¹ is independently —C(O)NH_{2.}

In embodiments, R¹ is —SSR^{1D}.

In embodiments, R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, substituted or unsubstituted alkyl (e.g., C₁-C₈, C₁-C₆, or C₁-C₄), or substituted or unsubstituted heteroalkyl (e.g., 2 to 10 membered, 2 to 6 membered, or 2 to 4 membered).

In embodiments, R^{ig} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted alkyl (e.g., C₁-C₈, C₁-C₆, or C₁-C₄), or R¹⁰-substituted or unsubstituted heteroalkyl (e.g., 2 to 10 membered, 2 to 6 membered, or 2 to 4 membered).

In embodiments, R^{ig} is independently substituted or unsubstituted alkyl. In embodiments, R^{ig} is independently R¹⁰-substituted or unsubstituted alkyl. In embodiments, R^{1D} is independently R¹⁰-substituted or unsubstituted C₁-C₁₆ alkyl. In embodiments, R^{1D} independently unsubstituted C₁-C₁₆ alkyl.

In embodiments, R¹ is —SR^{1D}, —NR^{1A}R^{1B}, OR^{1D}, E, unsubstituted alkyl, substituted or unsubstituted phenyl, or substituted or unsubstituted 5 to 6 membered heteroaryl; R^{1A} is independently hydrogen or unsubstituted C₁-C₄ alkyl; R^{1B} is independently hydrogen or unsubstituted C₁-C₄ alkyl; and R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, or substituted or unsubstituted alkyl (e.g., C₁-C₈, C₁-C₆, or C₁-C₄).

In embodiments, R¹ is —SR^{1D}, —NR^{1A}R^{1B}, —OR^{1D}, E, unsubstituted alkyl, substituted or unsubstituted phenyl, or substituted or unsubstituted 5 to 6 membered heteroaryl. In embodiments, R^{1A} is independently hydrogen or unsubstituted C₁-C₄ alkyl. In embodiments, R^{1B} is independently hydrogen or unsubstituted C₁-C₄ alkyl. In embodiments, R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, or substituted or unsubstituted alkyl (e.g., C₁-C₈, C₁-C₆, or C₁-C₄).

In embodiments, R¹ is —SR^{1D}, —NR^{1A}R^{1B}, —OR^{1D}, E, unsubstituted C₁-C₄ alkyl, R¹⁰-substituted or unsubstituted phenyl, or R¹⁰-substituted or unsubstituted 5 to 6 membered heteroaryl; R^{1A} is independently hydrogen or unsubstituted C₁-C₄ alkyl; R^{1B} is independently hydrogen or unsubstituted C₁-C₄ alkyl; and R^{ig} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl.

In embodiments, R¹ is —SR^{1D}, —NR^{1A}R^{1B}, —OR^{1D}, E, unsubstituted C₁-C₄ alkyl, R¹⁰-substituted or unsubstituted phenyl, or R¹⁰-substituted or unsubstituted 5 to 6 membered heteroaryl. In embodiments, R^{1A} is independently hydrogen or unsubstituted C₁-C₄ alkyl. In embodiments, R^{1B} is independently hydrogen or unsubstituted C₁-C₄ alkyl. In embodiments, R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl.

R¹⁰ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is —SR^{1D} or R¹⁰-substituted phenyl; R^{ig} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl; and R¹⁰ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

In embodiments, R¹ is —SR^{1D} or R¹⁰-substituted phenyl. In embodiments, R^{1D} is independently hydrogen, halogen, —CCl₃, —CBr₃, —CF₃, —CI₃, —CH₂Cl, —CH₂Br, —CH₂I, —CHCl₂, —CHBr₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl. In embodiments, le° is independently oxo, halogen, —CCl₃, —CBr₃, —CF₃, —CI₃, —CH₂Cl, —CH₂Br, —CH₂I, —CHCl₂, —CHBr₂, —CHI₂, —OCCl₃, —OCBr₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

In embodiments, R¹ is —SH, —SC(O)CH₃, or —SSCH₃. In embodiments, R¹ is —SH. In embodiments, R¹ is —SC(O)CH₃. In embodiments, R¹ is —SSCH₃.

In embodiments, R¹ is independently halogen, —NO₂, —SH, —SeH, —SO₃H, —SC(O)CH₃, —SSCH₃, —SP(O)(OH)₂, R¹⁰-substituted or unsubstituted heteroalkyl, or R¹⁰-substituted or unsubstituted heteroaryl; and R¹⁰ is as described herein, including in embodiments. In embodiments, R¹ is independently —F. In embodiments, R¹ is independently —Cl. In embodiments, R¹ is independently -Br. In embodiments, R¹ is independently —I. In embodiments, R¹ is independently —NO₂. In embodiments, R¹ is independently —SH. In embodiments, R¹ is independently —SeH. In embodiments, R¹ is independently —SO₃H. In embodiments, R¹ is independently —SC(O)CH₃. In embodiments, R¹ is independently —SSCH₃. In embodiments, R¹ is independently —SP(O)(OH)₂. In embodiments, R¹ is independently an R¹⁰-substituted or unsubstituted heteroalkyl. In embodiments, R¹ is independently an R¹⁰-substituted or unsubstituted 2 to 20 membered heteroalkyl. In embodiments, R¹ is independently an R¹⁰-substituted 2 to 20 membered heteroalkyl. In embodiments, R¹ is independently an unsubstituted 2 to 20 membered heteroalkyl. In embodiments, R¹ is independently —S-(C₁-C₂₀ alkyl). In embodiments, R¹ is independently —SCH₃. In embodiments, R¹ is independently —S(O)₂CH₃. In embodiments, R¹ is independently

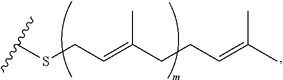

wherein m is independently an integer from 0 to 4. In embodiments, R¹ is independently

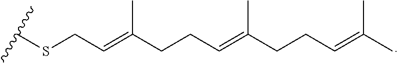 [SMILES: *SC/C=C(\C)CC/C=C(\C)CCC=C(C)C]

In embodiments, R¹ is independently —Si(CH₃)₃. In embodiments, R¹ is independently —Si(CH₂CH₃)₃. In embodiments, R¹ is independently —Si(CH₂CH₂CH₃)₃. In embodiments, R¹ is independently —Si(CH(CH₃)₂)₃. In embodiments, R¹ is independently —Si(CH₂CH₂CH₂CH₃)₃. In embodiments, R¹ is independently —Si(C(CH₃)₃)₃. In embodiments, R¹ is independently an R¹⁰-substituted or unsubstituted heteroaryl. In embodiments, R¹ is independently an R¹⁰-substituted or unsubstituted 5 to 10 membered heteroaryl. In embodiments, R¹ is independently an unsubstituted 5 to 10 membered heteroaryl. In embodiments, R¹ is independently an unsubstituted thiophenyl. In embodiments, R¹ is independently an unsubstituted furanyl. In embodiments, R¹ is independently an unsubstituted pyrrolyl. In embodiments, R¹ is independently an unsubstituted imidazolyl. In embodiments, R¹ is independently an unsubstituted tetrazolyl.

In embodiments, R¹ is independently

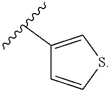 [SMILES: *c1ccsc1]

In embodiments, R¹ is independently

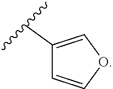 [SMILES: *c1ccoc1]

In embodiments, R¹ is independently

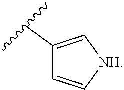 [SMILES: *c1cc[nH]c1]

In embodiments, R¹ is R¹⁰-substituted phenyl, and R′° is independently halogen. In embodiments, R¹

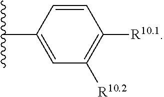

wherein R^{10.0} and R^{10.2} may each independently be hydrogen or any value of R¹⁰ as described herein, including in embodiments. In embodiments, R^{10.1} and R^{10.2} are each independently halogen. In embodiments, R¹ is

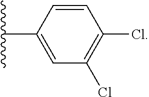 [SMILES: *c1ccc(Cl)c(Cl)c1]

In embodiments, R¹ is independently an R¹⁰-substituted or unsubstituted 2 to 8 membered heteroalkyl, and R¹⁰ is as described herein, including in embodiments. In embodiments, R¹ is independently an R¹⁰-substituted 2 to 8 membered heteroalkyl, and R¹⁰ is independently oxo. In embodiments, R¹ is independently —NHC(O)—(R¹⁰-substituted or unsubstituted C₁-C₄ alkyl). In embodiments, R¹ is independently —NHC(O)-(R¹⁰-substituted C₁-C₄ alkyl). In embodiments, R¹ is independently —NHC(O)-(unsubstituted C₁-C₄ alkyl). In embodiments, R¹ is independently

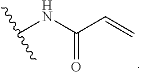 [SMILES: *NC(=O)C=C]

In embodiments, R¹ is independently

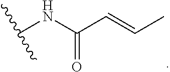 [SMILES: *NC(=O)/C=C/C]

In embodiments, R¹ is independently

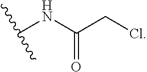 [SMILES: *NC(=O)CCl]

In embodiments, R¹ is independently

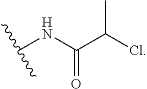 [SMILES: *NC(=O)C(C)Cl]

In embodiments, R¹ is independently —NHS(O)₂-(unsubstituted C₁-C₄ alkyl). In embodiments, R¹ is independently

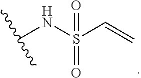 [SMILES: *NS(=O)(=O)C=C]

In embodiments, R¹ is E

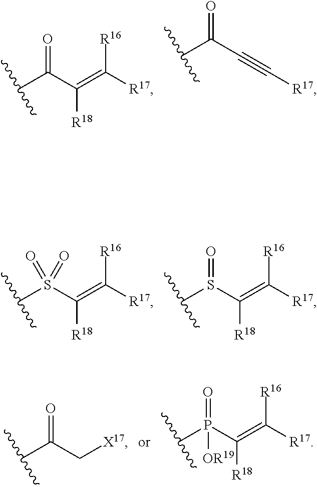

R¹⁶ is independently hydrogen, halogen, —CX¹⁶₃, —CHX¹⁶₂, —CH₂X¹⁶, —CN, —SOₙ₁₆R^{16A}, —SOᵥ₁₆NR^{16A}R^{16B}, NHNR^{16A}R^{16B}, —ONR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶₂, —OCH₂X¹⁶, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁷ is independently hydrogen, halogen, —CX¹⁷₃, —CHX¹⁷₂, —CH₂X¹⁷, —CN, —SOₙ₁R^{17A}, —SOᵥ₁₇NR^{17A}R^{17B}, —NHNR^{17A}R^{17B}, —ONR^{17A}R^{17B}, NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OCH₂X¹⁷, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁸ is independently hydrogen, halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{1B}, —ONR^{18A}R^{18B}, —NHC(O)NHNR^{18A}R^{18B}, —NHC(O)NR^{18A}R^{18B}, —N(O)ₘ₁₈, —NR^{18A}R^{18B}, —C(O)R^{18A}, —C(O)—OR^{18A}, —C(O)NR^{18A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX^{18B}, —OCHX¹⁸₂, —OCH₂X¹⁸, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁹ is independently hydrogen, halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B}, —C(O)R^{19A}, —C(O)—OR^{19A}, —C(O)NR^{19A}R^{19B}, —OR^{19A}, —NR^{19A}SO₂R^{19B}, —NR^{19A}C(O)R^{19B}, —NR^{19A}C(O)OR^{19B}, —NR^{19A}OR^{19B}, —OCX¹⁹₃, —OCHX¹⁹₂, —OCH₂X¹⁹, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{16A}, R^{16B}, R^{17A}, R^{17B}, R^{18A}, R^{18B}, R^{19A}, and R^{19B} are independently hydrogen, —CX₃, —CHX₂, —CH₂X, —CN, —OH, —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{16A} and R¹substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; R^{19A} and R^{19B} substituents bonded to the same nitrogen atom may optionally be joined to form a substituted or unsubstituted heterocycloalkyl or substituted or unsubstituted heteroaryl.

X, X¹⁶, X¹⁷, X¹⁸, and X¹⁹ are independently —F, —Cl, —Br, or —I.

n16, n17, n18, and n19 are independently an integer from 0 to 4.

m16, m17, m18, m19, v16, v17, v18, and v19 are independently 1 or 2.

In embodiments, R¹⁶ is independently hydrogen, halogen, —CX¹⁶₃, —CHX¹⁶₂, —CH₂X¹⁶, —CN, —SOₙ₁R^{16A}, —SOᵥ₁₆NR^{16A}R^{16B}, NHNR^{16A}R^{16B}, —NR^{16A}R^{16B}, —NHC(O)NHNR^{16A}R^{16B}, —NHC(O)NR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, —NR^{16A}SO₂R^{16B}, —NR^{16A}C(O)R^{16B}, —NR^{16A}C(O)OR^{16B}, —NR^{16A}OR^{16B}, —OCX16₃, —OCHX¹⁶₂, —OCH₂X¹⁶, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, R¹⁶ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁷ is independently hydrogen, halogen, —CX¹⁷₃, —CHX¹⁷₂, —CH₂X¹⁷, —CN, —SOₙ₁R^{17A}, —SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}—NR^{17A}SO₂R^{17B}, —NR^{17A}C(O)_{R}^{17B}, —NR^{17A}C(O)OR^{17B}, —NR^{17A}OR^{17B}, —OCX¹⁷₃, —OCHX¹⁷₂, —OCH₂X¹⁷, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF_{2,} ⁻OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁸ is independently hydrogen, halogen, —CX¹⁸₃, —CHX¹⁸₂, —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{18B}, —C(O)R^{18A}, —C(O)—OR^{18A}, —C(O)NR^{8A}R^{18B}, —OR^{18A}, —NR^{18A}SO₂R^{18B}, —NR^{18A}C(O)R^{18B}, —NR^{18A}C(O)OR^{18B}, —NR^{18A}OR^{18B}, —OCX¹⁸₃, —OCHX¹⁸₂, —OCH₂X¹⁸, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, R¹⁸ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁹ is independently hydrogen, halogen, —CX¹⁹3, —CHX¹⁹2, —CH₂X¹⁹, —CN, —SOₙ₁₉R^{19A}, —SOᵥ₁₉NR^{19A}R^{19B}, NHNR^{19A}R^{19B}, —ONR^{19A}R^{19B}, —NHC(O)NHNR^{19A}R^{19B}, NHC(O)NR^{19A}R^{19B}, —N(O)ₘ₁₉, —NR^{19A}R^{19B}, —C(O)_{R}^{19A}, —C(O)—OR^{19A}, —C(O)NR^{19A}R^{19B}, —OR^{19A}, —NR^{19A}SO₂R^{19B}, —NR^{19A}C(O)_{R}^{19B}, —NR^{19A}C(O)OR^{19B}, —NR^{19A}OR^{19B}, —OCX¹⁹₃, —OCHX¹⁹₂, —OCH₂X¹⁹, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, R¹⁹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R^{16A} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{16A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16A} is substituted with a plurality of groups selected from substituent groups, size-limited substituent groups, and lower substituent groups; each substituent group, size-limited substituent group, and/or lower substituent group may optionally be different. In embodiments, when R^{16A} is substituted, it is substituted with at least one substituent group. In embodiments, when R^{16A} is substituted, it is substituted with at least one size-limited substituent group. In embodiments, when R^{16A} is substituted, it is substituted with at least one lower substituent group.

In embodiments, R^{16B} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{16B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{16B} is substituted with a plurality of groups selected from substituent groups, size-limited substituent groups, and lower substituent groups; each substituent group, size-limited substituent group, and/or lower substituent group may optionally be different. In embodiments, when R^{16B} is substituted, it is substituted with at least one substituent group. In embodiments, when R^{16B} is substituted, it is substituted with at least one size-limited substituent group. In embodiments, when R^{16B} is substituted, it is substituted with at least one lower substituent group.

In embodiments, R^{16A} and R^{16B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl.

In embodiments, a substituted moiety formed by joining R^{16A} and R^{16B} substituents bonded to the same nitrogen atom (e.g., substituted heterocycloalkyl and/or substituted heteroaryl) is substituted with at least one substituent group, size-limited substituent group, or lower substituent group; wherein if the substituted moiety formed by joining R^{16A} and R^{16B} substituents bonded to the same nitrogen atom is substituted with a plurality of groups selected from substituent groups, size-limited substituent groups, and lower substituent groups; each substituent group, size-limited substituent group, and/or lower substituent group may optionally be different. In embodiments, when the moiety formed by joining R^{16A} and R^{16B} substituents bonded to the same nitrogen atom is substituted, it is substituted with at least one substituent group. In embodiments, when the moiety formed by joining R^{16A} and R^{16B} substituents bonded to the same nitrogen atom is substituted, it is substituted with at least one size-limited substituent group. In embodiments, when the moiety formed by joining R^{16A} and R^{16B} substituents bonded to the same nitrogen atom is substituted, it is substituted with at least one lower substituent group.

In embodiments, R^{17A} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group.

In embodiments, R^{17B} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{17B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17B} is substituted with a plurality of groups selected from substituent groups, size-limited substituent groups, and lower substituent groups; each substituent group, size-limited substituent group, and/or lower substituent group may optionally be different. In embodiments, when R^{17B} is substituted, it is substituted with at least one substituent group. In embodiments, when R^{17B} is substituted, it is substituted with at least one size-limited substituent group. In embodiments, when R^{17B} is substituted, it is substituted with at least one lower substituent group.

In embodiments, R^{17A} and R^{17B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl.

In embodiments, a substituted moiety formed by joining R^{17A} and R^{17B} substituents bonded to the same nitrogen atom (e.g., substituted heterocycloalkyl and/or substituted heteroaryl) is substituted with at least one substituent group, size-limited substituent group, or lower substituent group; wherein if the substituted moiety formed by joining R^{17A} and R^{17B} substituents bonded to the same nitrogen atom is substituted with a plurality of groups selected from substituent groups, size-limited substituent groups, and lower substituent groups; each substituent group, size-limited substituent group, and/or lower substituent group may optionally be different. In embodiments, when the moiety formed by joining R^{17A} and R^{17B} substituents bonded to the same nitrogen atom is substituted, it is substituted with at least one substituent group. In embodiments, when the moiety formed by joining R^{17A} and R^{17B} substituents bonded to the same nitrogen atom is substituted, it is substituted with at least one size-limited substituent group. In embodiments, when the moiety formed by joining

R^{17A} and R^{17B} substituents bonded to the same nitrogen atom is substituted, it is substituted with at least one lower substituent group.

In embodiments, R^{18A} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or

C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{18A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18A} is substituted with a plurality of groups selected from substituent groups, size-limited substituent groups, and lower substituent groups; each substituent group, size-limited substituent group, and/or lower substituent group may optionally be different. In embodiments, when R^{18A} is substituted, it is substituted with at least one substituent group. In embodiments, when R^{18A} is substituted, it is substituted with at least one size-limited substituent group. In embodiments, when R^{18A} is substituted, it is substituted with at least one lower substituent group.

In embodiments, R^{11B} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{18B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{18B} is substituted with a plurality of groups selected from substituent groups, size-limited substituent groups, and lower substituent groups; each substituent group, size-limited substituent group, and/or lower substituent group may optionally be different. In embodiments, when R^{18B} is substituted, it is substituted with at least one substituent group. In embodiments, when R^{18B} is substituted, it is substituted with at least one size-limited substituent group. In embodiments, when R^{18B} is substituted, it is substituted with at least one lower substituent group.

In embodiments, R^{18A} and R^{18B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl.

In embodiments, a substituted moiety formed by joining R^{18A} and R^{18B} substituents bonded to the same nitrogen atom (e.g., substituted heterocycloalkyl and/or substituted heteroaryl) is substituted with at least one substituent group, size-limited substituent group, or lower substituent group; wherein if the substituted moiety formed by joining R^{18A} and R^{18B} substituents bonded to the same nitrogen atom is substituted with a plurality of groups selected from substituent groups, size-limited substituent groups, and lower substituent groups; each substituent group, size-limited substituent group, and/or lower substituent group may optionally be different. In embodiments, when the moiety formed by joining R^{18A} and R^{18B} substituents bonded to the same nitrogen atom is substituted, it is substituted with at least one substituent group. In embodiments, when the moiety formed by joining R^{18A} and R^{18B} substituents bonded to the same nitrogen atom is substituted, it is substituted with at least one size-limited substituent group. In embodiments, when the moiety formed by joining R^{18A} and R^{18B} substituents bonded to the same nitrogen atom is substituted, it is substituted with at least one lower substituent group.

In embodiments, R^{19A} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{19A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19A} is substituted with a plurality of groups selected from substituent groups, size-limited substituent groups, and lower substituent groups; each substituent group, size-limited substituent group, and/or lower substituent group may optionally be different. In embodiments, when R^{19A} is substituted, it is substituted with at least one substituent group. In embodiments, when R^{19A} is substituted, it is substituted with at least one size-limited substituent group. In embodiments, when R^{19A} is substituted, it is substituted with at least one lower substituent group.

In embodiments, R^{19B} is independently hydrogen, —CX₃, —CHX₂, —CH₂X, —CN, —OH, —COOH, —CONH₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{19B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{19B} is substituted with a plurality of groups selected from substituent groups, size-limited substituent groups, and lower substituent groups; each substituent group, size-limited substituent group, and/or lower substituent group may optionally be different. In embodiments, when R^{19B} is substituted, it is substituted with at least one substituent group. In embodiments, when R^{19B} is substituted, it is substituted with at least one size-limited substituent group. In embodiments, when R^{19B} is substituted, it is substituted with at least one lower substituent group.

In embodiments, R^{19A} and R^{19B} substituents bonded to the same nitrogen atom may optionally be joined to form a substituted (e.g., substituted with at least one substituent group, size-limited substituent group, or lower substituent group) or unsubstituted heterocycloalkyl or substituted (e.g., substituted with at least one substituent group, size-limited substituent group, or lower substituent group) or unsubstituted heteroaryl.

In embodiments, a substituted moiety formed by joining R^{19A} and R^{19B} substituents bonded to the same nitrogen atom (e.g., substituted heterocycloalkyl and/or substituted heteroaryl) is substituted with at least one substituent group, size-limited substituent group, or lower substituent group; wherein if the substituted moiety formed by joining R^{19A} and R^{19B} substituents bonded to the same nitrogen atom is substituted with a plurality of groups selected from substituent groups, size-limited substituent groups, and lower substituent groups; each substituent group, size-limited substituent group, and/or lower substituent group may optionally be different. In embodiments, when the moiety formed by joining R^{19A} and R^{19B} substituents bonded to the same nitrogen atom is substituted, it is substituted with at least one substituent group. In embodiments, when the moiety formed by joining R^{19A} and R^{19B} substituents bonded to the same nitrogen atom is substituted, it is substituted with at least one size-limited substituent group. In embodiments, when the moiety formed by joining R^{19A} and R^{19B} substituents bonded to the same nitrogen atom is substituted, it is substituted with at least one lower substituent group.

In embodiments, R^{16A}, R^{16B}, R^{17A}, R^{17B}, R^{18A}, R^{18B}, R^{19A}, and R^{19B} are independently hydrogen, —CCl₃, —CBr₃, —CF₃, —CI₃, —CH₂Cl, —CH₂Br, —CH₂F, —CHCl₂, —CHBr₂, —CHF₂, —CN, —OH, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); R^{16A} and R^{16B} substituents bonded to the same nitrogen atom may optionally be joined to form an unsubstituted heterocycloalkyl or unsubstituted heteroaryl; R^{17A} and R^{17B} substituents bonded to the same nitrogen atom may optionally be joined to form an unsubstituted heterocycloalkyl or unsubstituted heteroaryl; R^{18A} and R^{18B} substituents bonded to the same nitrogen atom may optionally be joined to form an unsubstituted heterocycloalkyl or unsubstituted heteroaryl; R^{19A} and R^{19B} substituents bonded to the same nitrogen atom may optionally be joined to form an unsubstituted heterocycloalkyl or unsubstituted heteroaryl.

In embodiments, E is

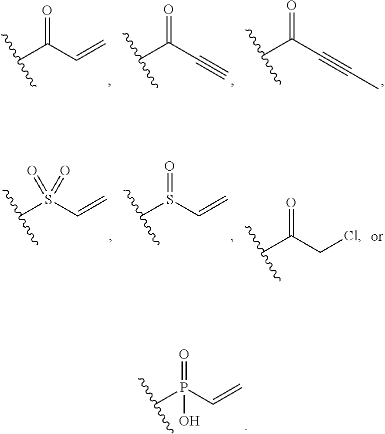

In embodiments, E is

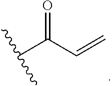 [SMILES: *C(=O)C=C]

In embodiments, E is

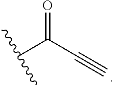 [SMILES: *C(=O)C#C]

In embodiments, E is

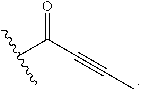 [SMILES: *C(=O)C#CC]

In embodiments, E is

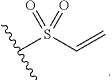 [SMILES: *S(=O)(=O)C=C]

In embodiments, E is

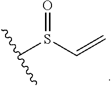 [SMILES: *S(=O)C=C]

In embodiments, E is

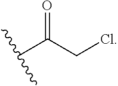 [SMILES: *C(=O)CCl]

In embodiments, E is

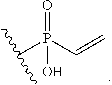 [SMILES: *P(=O)(O)C=C]

In embodiments, the compound has the formula

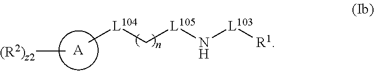

Ring A, R¹, R², L¹⁰³, L¹⁰⁴, L¹⁰⁵ and z2 are as described herein.

In embodiments, L¹⁰³ is a bond, substituted or unsubstituted alkylene, or substituted or unsubstituted heteroalkylene.

In embodiments, L¹⁰⁴ is a bond, —O—, —NH—, —S—, or substituted or unsubstituted alkylene.

In embodiments, L¹⁰⁵ is —S(O)₂—, —C(O)—, —NHC(O)—, —C(O)NH—, —OC(O)—, or —C(O)O—.

In embodiments, L¹⁰⁵ is —S(O)₂—, —C(O)—, —NHC(O)—, or —OC(O)—.

n is an integer from 0 to 4.

In embodiments, the compound has the formula

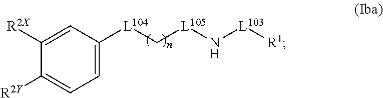

R¹, R^{2X}, R^{2Y}, R¹⁰³, R¹⁰⁴, L¹⁰⁵, n, and z2 are as described herein. In embodiments, L¹⁰³ is a bond, substituted or unsubstituted C₁-C₆ alkylene, or substituted or unsubstituted 2 to 6 membered heteroalkylene; L¹⁰⁴ is a bond, —O—, —NH—, —S—, or substituted or unsubstituted C₁-C₄ alkylene; L¹⁰⁵ is —S(O)₂—, —C(O)—, —NHC(O)—, or —OC(O)—; n is an integer from 0 to 4; and R^{2X} and R^{2Y} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}_{R}^{2B}, —NHC(O)NR^{2A}R^{2D}, —N(O)ₘ₂, —NR^{2A}R^{2B}, —C(O)R^{2C}, —C(O)OR^{2C}, —C(O)NR^{2A}R^{2D}, —OR^{2D}, —NR^{2A}SO₂R^{2D}, —NR^{2A}C(O)_{R}^{2C} —NR^{2A}C(O)OR^{2C}, —R^{2A}OR^{2C}, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2X} and R^{2Y} substituents may be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, L¹⁰³ is a bond, substituted or unsubstituted C₁-C₆ alkylene, or substituted or unsubstituted 2 to 6 membered heteroalkylene; L¹⁰⁴ is a bond, —O—, —NH—, —S—, or substituted or unsubstituted C₁-C₄ alkylene; L¹⁰⁵ is —S(O)₂—, —C(O)—, —NHC(O)—, or —OC(O)—; n is an integer from 0 to 4; and R^{2X} and R^{2Y} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}_{R}^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —NR^{2A}SO₂R²², —NR^{2A}C(O)R^{2C}, —NR^{2A}C(O)OR^{2C}, —NR^{2A}OR^{2C}, —N₃, R²⁰-substituted or unsubstituted alkyl (e.g., C₁-C₈, C₁-C₆, or C₁-C₄), R²⁰-substituted or unsubstituted heteroalkyl (e.g., 2 to 8 membered, 2 to 6 membered, or 2 to 4 membered), R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered); R^{2X} and R^{2Y} substituents may be joined to form an R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered).

In embodiments, n is 0. In embodiments, n is 1. In embodiments, n is 2. In embodiments, n is 3. In embodiments, n is 4. In embodiments, when n is 0, L¹⁰⁴ and L¹⁰⁵ are not a bond.

In embodiments, R^{2X} and R^{2Y} are independently halogen. In embodiments, R^{2X} and R^{2Y} are independently —Cl.

In embodiments, L¹⁰³ is an unsubstituted alkylene. In embodiments, L¹⁰³ is an unsubstituted C₁-C₆ alkylene. In embodiments, L¹⁰³ is an unsubstituted C₁-C₄ alkylene. In embodiments, L¹⁰³ is a bond.

In embodiments, L¹⁰⁴ is a bond, —O—, —NH—, —S—, or substituted or unsubstituted alkylene (e.g., C₁-C₈, C₁-C₆, or C₁-C₄). In embodiments, L¹⁰⁴ is a bond. In embodiments, L¹⁰⁴ is —O—. In embodiments, L¹⁰⁴ is —NH—. In embodiments, L¹⁰⁴ is —S—. In embodiments, L¹⁰⁴ is substituted or unsubstituted C₁-C₈ alkylene. In embodiments, L¹⁰⁴ is substituted or unsubstituted C₁-C₆ alkylene. In embodiments, L¹⁰⁴ is substituted or unsubstituted C₁-C₄ alkylene. In embodiments, L¹⁰⁴ is unsubstituted C₁-C₈ alkylene.

In embodiments, L¹⁰⁴ is a bond, —O—, —NH—, —S—, or R¹⁰⁴-substituted or unsubstituted alkylene (e.g., C₁-C₈, C₁-C₆, or C₁-C₄). R¹⁰⁴ is as described herein, including in embodiments.

In embodiments, L¹⁰⁵ is —S(O)₂—, —C(O)—, —NHC(O)—, or —OC(O)—. In embodiments, L¹⁰⁵ is —S(O)₂—. In embodiments, L¹⁰⁵ is —C(O)—. In embodiments, L¹⁰⁵ is —NHC(O)—. In embodiments, L¹⁰⁵ is —OC(O)—.

In embodiments, -L¹⁰⁴-CH₂-L¹⁰⁵-NH-L¹⁰³—is

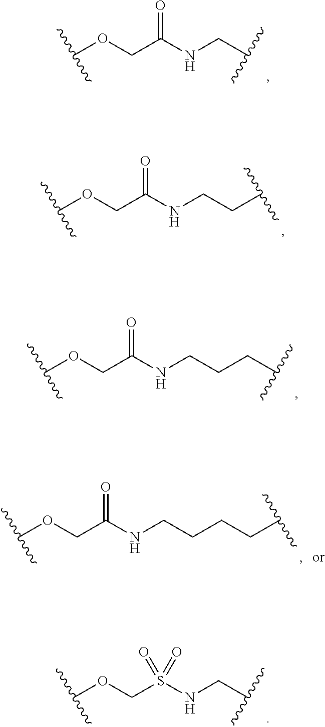 [SMILES: *CNC(=O)CO* ; *CCNC(=O)CO* ; *CCCNC(=O)CO* ; *CCCCNC(=O)CO* ; *CNS(=O)(=O)CO*]

In embodiments, -L¹⁰⁴-CH₂-L¹⁰⁵-NH-L¹⁰³—is

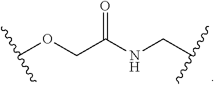 [SMILES: *CNC(=O)CO*]

In embodiments, -L¹⁰⁴-CH₂-L¹⁰⁵-NH-L¹⁰³—is

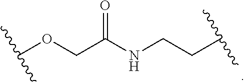 [SMILES: *CCNC(=O)CO*]

In embodiments, -L¹⁰⁴-CH₂-L¹⁰⁵-NH-L¹⁰³—is

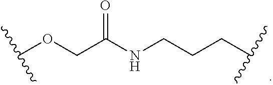 [SMILES: *CCCNC(=O)CO*]

In embodiments, -L¹⁰⁴-CH₂-L¹⁰⁵-NH-L¹⁰³—is

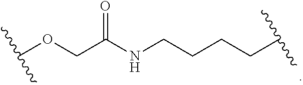 [SMILES: *CCCCNC(=O)CO*]

In embodiments, -L¹⁰⁴-CH₂-L¹⁰⁵-NH-L¹⁰³—is

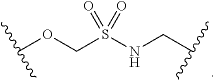 [SMILES: *CNS(=O)(=O)CO*]

In embodiments, -L¹⁰⁴-CH₂-L¹⁰⁵-NH-L¹⁰³—is

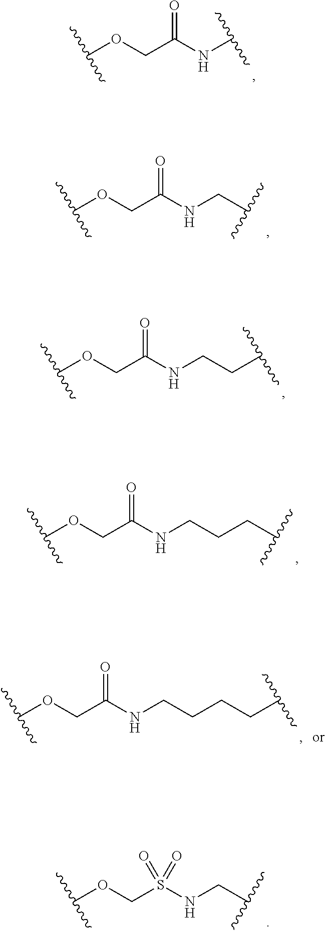 [SMILES: *NC(=O)CO* ; *CNC(=O)CO* ; *CCNC(=O)CO* ; *CCCNC(=O)CO* ; *CCCCNC(=O)CO* ; *CNS(=O)(=O)CO*]

In embodiments, -L¹⁰⁴-CH₂-L¹⁰⁵-NH-L¹⁰³—is

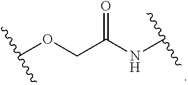 [SMILES: *NC(=O)CO*]

In embodiments, R¹ is hydrogen, —SR^{1D}, —NR^{1A}R^{1B}, —OR^{1D}, —NR^{1A}SO₂R^{1D}, —NR^{1A}C(O)R^{1C}, E, substituted or unsubstituted C₁-C₆ alkyl, substituted or unsubstituted 2 to 10 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl; E is an electrophilic moiety; R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂—CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted C₁-C₆ alkyl, substituted or unsubstituted 2 to 6 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl.

In embodiments, R¹ is hydrogen, —SR^{1D}, —NR^{1A}R^{1B}, —OR^{1D}, —NR^{1A}SO₂R^{1D}, —NR^{1A}C(O)R^{1C}, E, R¹⁰-substituted or unsubstituted C₁-C₆ alkyl, R¹⁰-substituted or unsubstituted 2 to 10 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl. In embodiments, R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹⁰-substituted or unsubstituted C₁-C₆ alkyl, R¹⁰-substituted or unsubstituted 2 to 10 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl.

In embodiments, R¹⁰ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R″-substituted or unsubstituted alkyl (e.g., C₁-C₈, C₁-C₆, or C₁-C₄), R¹¹-substituted or unsubstituted heteroalkyl (e.g., 2 to 8 membered, 2 to 6 membered, or 2 to 4 membered), R¹¹-substituted or unsubstituted cycloalkyl (e.g., C₃-C₈, C₃⁻C₆, or C₅-C₆), R¹¹-substituted or unsubstituted heterocycloalkyl (e.g., 3 to 8 membered, 3 to 6 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).

R¹¹ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R¹²-substituted or unsubstituted alkyl (e.g., C₁-C₈, C i-C 6, or C₁-C₄), R¹²-substituted or unsubstituted heteroalkyl (e.g., 2 to 8 membered, 2 to 6 membered, or 2 to 4 membered), R¹²-substituted or unsubstituted cycloalkyl (e.g., C₃-C₈, C₃-C₆, or C₅-C₆), R¹²-substituted or unsubstituted heterocycloalkyl (e.g., 3 to 8 membered, 3 to 6 membered, or 5 to 6 membered), R¹²-substituted or unsubstituted aryl (e.g., C₆-C₁₀ or phenyl), or R¹²-substituted or unsubstituted heteroaryl (e.g., 5 to 10 membered, 5 to 9 membered, or 5 to 6 membered).

R¹² is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R¹³-substituted or unsubstituted alkyl (e.g., C₁-C₈, C₁-C₆, or C₁-C₄), R¹³-substituted or unsubstituted heteroalkyl (e.g., 2 to 8 membered, 2 to 6 membered, or 2 to 4 membered), R¹³-substituted or unsubstituted cycloalkyl (e.g., C₃-C₈, C₃-C₆, or C₅-C₆), R¹³-substituted or unsubstituted heterocycloalkyl (e.g., 3 to 8 membered, 3 to 6 membered, or 5 to 6 membered), R¹³-substituted or unsubstituted aryl (e.g., C₆-C₁₀ or phenyl), or 10³-substituted or unsubstituted heteroaryl (e.g., 5 to 10 membered, 5 to 9 membered, or 5 to 6 membered).

R¹³ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}_{R}^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}—OR^{2D}, —NR^{2A}SO₂R^{2D}, —NR^{2A}C(O)R^{2C}, —NR^{2A}C(O)OR^{2C}, —NR^{2A}OR^{2C},—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

In embodiments, R² is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R² substituents bonded to adjacent atoms may be joined to form an substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R² is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R²⁰-substituted or unsubstituted alkyl (e.g., C₁-C₈, C₁-C₆, or C₁-C₄), R²⁰-substituted or unsubstituted heteroalkyl (e.g., 2 to 8 membered, 2 to 6 membered, or 2 to 4 membered), R²⁰-substituted or unsubstituted cycloalkyl (e.g., C₃-C₈, C₃-C₆, or C₅-C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered); two R² substituents bonded to adjacent atoms may be joined to form an R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl.

R²⁰ is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, R²¹-substituted or unsubstituted alkyl (e.g., C₁-C₈, C₁-C₆, or C₁-C₄), R²¹-substituted or unsubstituted heteroalkyl (e.g., 2 to 8 membered, 2 to 6 membered, or 2 to 4 membered), R²¹-substituted or unsubstituted cycloalkyl (e.g., C₃-C₈, C₃-C₆, or C₅-C₆), R²¹-substituted or unsubstituted heterocycloalkyl (e.g., 3 to 8 membered, 3 to 6 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered).

R²¹ is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, R²²-substituted or unsubstituted alkyl (e.g., C₁-C₈, C₁-C₆, or C₁-C₄), R²²-substituted or unsubstituted heteroalkyl (e.g., 2 to 8 membered, 2 to 6 membered, or 2 to 4 membered), R²²-substituted or unsubstituted cycloalkyl (e.g., C₃-C₈, C₃-C₆, or C₅-C₆), R²²-substituted or unsubstituted heterocycloalkyl (e.g., 3 to 8 membered, 3 to 6 membered, or 5 to 6 membered), R²²-substituted or unsubstituted aryl (e.g., C₆-C₁₀ or phenyl), or R²²-substituted or unsubstituted heteroaryl (e.g., 5 to 10 membered, 5 to 9 membered, or 5 to 6 membered).

R²² is independently oxo, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, the compound is

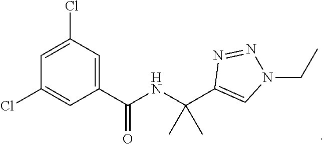 [SMILES: CCn1cc(C(C)(C)NC(=O)c2cc(Cl)cc(Cl)c2)nn1]

In embodiments, the compound is

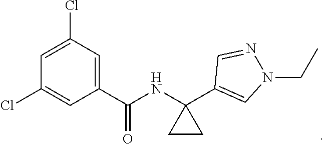 [SMILES: CCn1cc(C2(NC(=O)c3cc(Cl)cc(Cl)c3)CC2)cn1]

In embodiments, the compound is

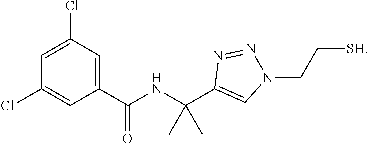 [SMILES: CC(C)(NC(=O)c1cc(Cl)cc(Cl)c1)c1cn(CCS)nn1]

In embodiments, the compound is

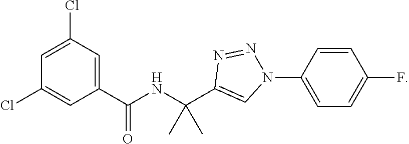 [SMILES: CC(C)(NC(=O)c1cc(Cl)cc(Cl)c1)c1cn(-c2ccc(F)cc2)nn1]

In embodiments, the compound is

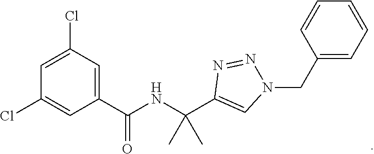 [SMILES: CC(C)(NC(=O)c1cc(Cl)cc(Cl)c1)c1cn(Cc2ccccc2)nn1]

In embodiments, the compound is

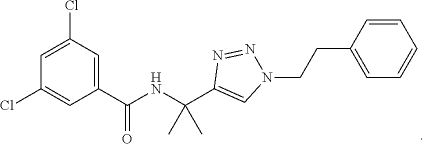 [SMILES: CC(C)(NC(=O)c1cc(Cl)cc(Cl)c1)c1cn(CCc2ccccc2)nn1]

In embodiments, the compound is

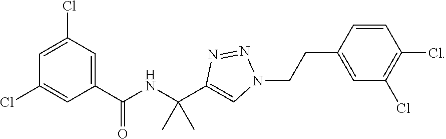 [SMILES: CC(C)(NC(=O)c1cc(Cl)cc(Cl)c1)c1cn(CCc2ccc(Cl)c(Cl)c2)nn1]

In embodiments, the compound is

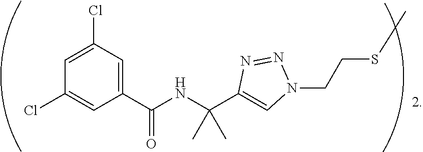

In embodiments, the compound is

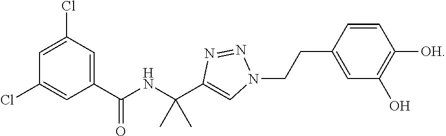 [SMILES: CC(C)(NC(=O)c1cc(Cl)cc(Cl)c1)c1cn(CCc2ccc(O)c(O)c2)nn1]

In embodiments, the compound is

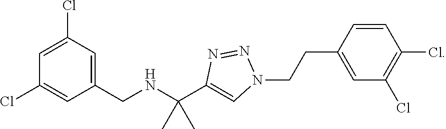 [SMILES: CC(C)(NCc1cc(Cl)cc(Cl)c1)c1cn(CCc2ccc(Cl)c(Cl)c2)nn1]

In embodiments, the compound is

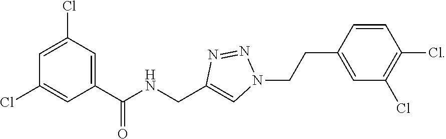 [SMILES: O=C(NCc1cn(CCc2ccc(Cl)c(Cl)c2)nn1)c1cc(Cl)cc(Cl)c1]

In embodiments, the compound is

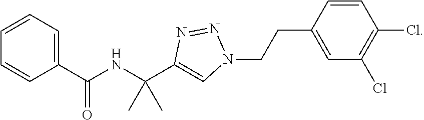 [SMILES: CC(C)(NC(=O)c1ccccc1)c1cn(CCc2ccc(Cl)c(Cl)c2)nn1]

In embodiments, the compound is

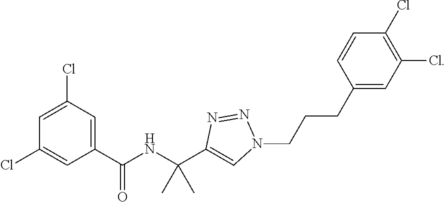 [SMILES: CC(C)(NC(=O)c1cc(Cl)cc(Cl)c1)c1cn(CCCc2ccc(Cl)c(Cl)c2)nn1]

In embodiments, the compound is

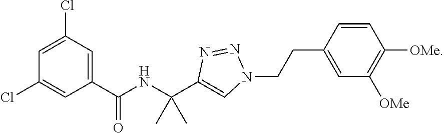 [SMILES: COc1ccc(CCn2cc(C(C)(C)NC(=O)c3cc(Cl)cc(Cl)c3)nn2)cc1OC]

In embodiments, the compound is

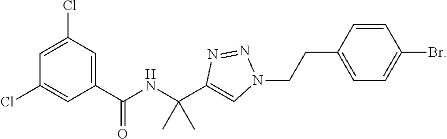 [SMILES: CC(C)(NC(=O)c1cc(Cl)cc(Cl)c1)c1cn(CCc2ccc(Br)cc2)nn1]

In embodiments, the compound is

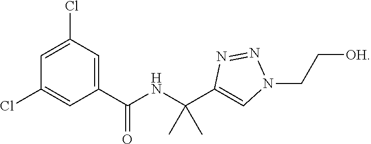 [SMILES: CC(C)(NC(=O)c1cc(Cl)cc(Cl)c1)c1cn(CCO)nn1]

In embodiments, the compound is

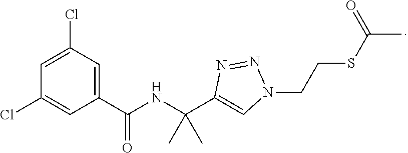 [SMILES: CC(=O)SCCn1cc(C(C)(C)NC(=O)c2cc(Cl)cc(Cl)c2)nn1]

In embodiments, the compound is

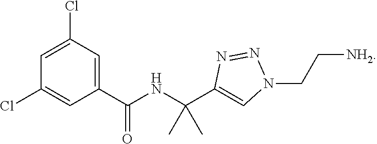 [SMILES: CC(C)(NC(=O)c1cc(Cl)cc(Cl)c1)c1cn(CCN)nn1]

In embodiments, the compound is

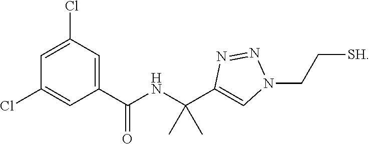 [SMILES: CC(C)(NC(=O)c1cc(Cl)cc(Cl)c1)c1cn(CCS)nn1]

In embodiments, the compound is

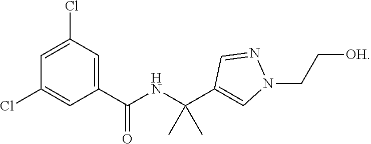 [SMILES: CC(C)(NC(=O)c1cc(Cl)cc(Cl)c1)c1cnn(CCO)c1]

In embodiments, the compound is

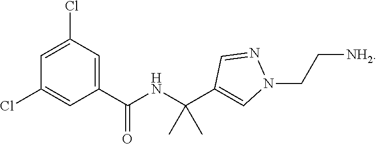 [SMILES: CC(C)(NC(=O)c1cc(Cl)cc(Cl)c1)c1cnn(CCN)c1]

In embodiments, the compound is

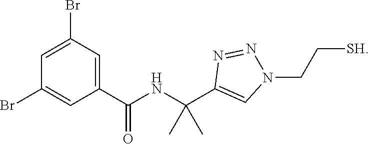 [SMILES: CC(C)(NC(=O)c1cc(Br)cc(Br)c1)c1cn(CCS)nn1]

In embodiments, the compound is

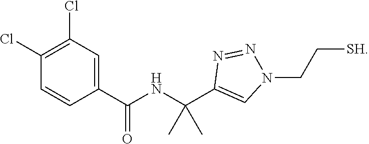 [SMILES: CC(C)(NC(=O)c1ccc(Cl)c(Cl)c1)c1cn(CCS)nn1]

In embodiments, the compound is

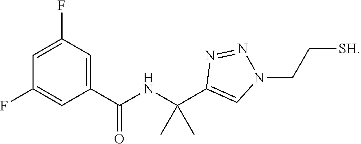 [SMILES: CC(C)(NC(=O)c1cc(F)cc(F)c1)c1cn(CCS)nn1]

In embodiments, the compound is

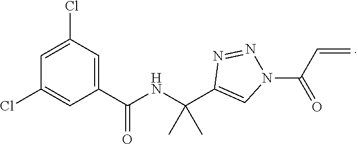 [SMILES: C=CC(=O)n1cc(C(C)(C)NC(=O)c2cc(Cl)cc(Cl)c2)nn1]

In embodiments, the compound is

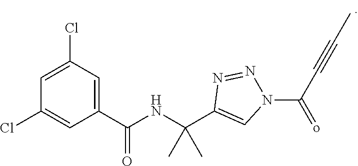

In embodiments, the compound is

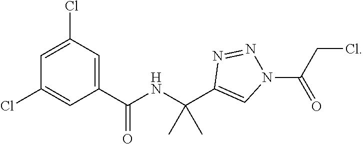 [SMILES: CC(C)(NC(=O)c1cc(Cl)cc(Cl)c1)c1cn(C(=O)CCl)nn1]

In embodiments, the compound is

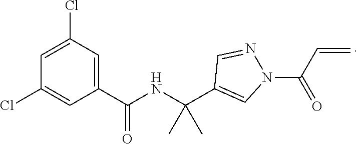 [SMILES: C=CC(=O)n1cc(C(C)(C)NC(=O)c2cc(Cl)cc(Cl)c2)cn1]

In embodiments, the compound is

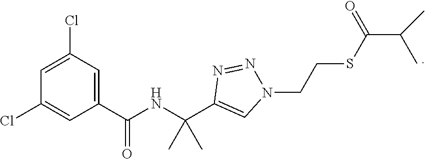 [SMILES: CC(C)C(=O)SCCn1cc(C(C)(C)NC(=O)c2cc(Cl)cc(Cl)c2)nn1]

In embodiments, the compound is

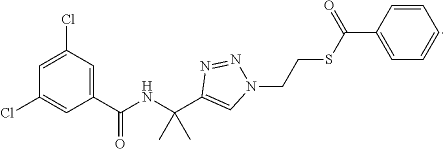 [SMILES: CC(C)(NC(=O)c1cc(Cl)cc(Cl)c1)c1cn(CCSC(=O)c2ccccc2)nn1]

In embodiments, the compound is

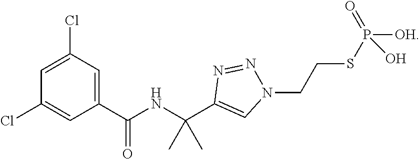 [SMILES: CC(C)(NC(=O)c1cc(Cl)cc(Cl)c1)c1cn(CCSP(=O)(O)O)nn1]

In embodiments, the compound is

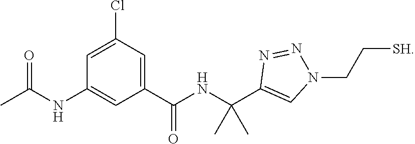 [SMILES: CC(=O)Nc1cc(Cl)cc(C(=O)NC(C)(C)c2cn(CCS)nn2)c1]

In embodiments, the compound is

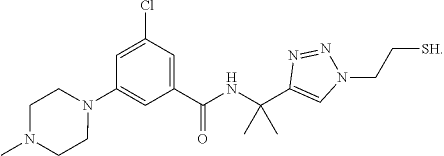 [SMILES: CN1CCN(c2cc(Cl)cc(C(=O)NC(C)(C)c3cn(CCS)nn3)c2)CC1]

In embodiments, the compound is

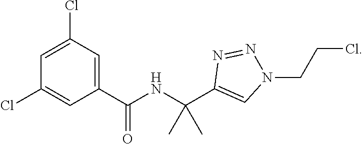 [SMILES: CC(C)(NC(=O)c1cc(Cl)cc(Cl)c1)c1cn(CCCl)nn1]

In embodiments, the compound is

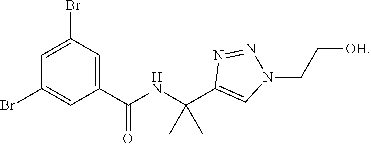 [SMILES: CC(C)(NC(=O)c1cc(Br)cc(Br)c1)c1cn(CCO)nn1]

In embodiments, the compound is

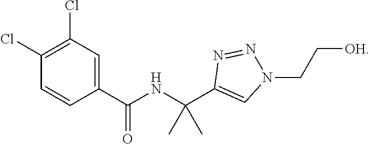 [SMILES: CC(C)(NC(=O)c1ccc(Cl)c(Cl)c1)c1cn(CCO)nn1]

In embodiments, the compound is

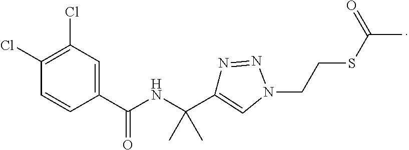 [SMILES: CC(=O)SCCn1cc(C(C)(C)NC(=O)c2ccc(Cl)c(Cl)c2)nn1]

In embodiments, the compound is

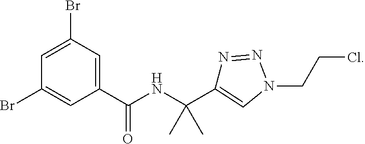 [SMILES: CC(C)(NC(=O)c1cc(Br)cc(Br)c1)c1cn(CCCl)nn1]

In embodiments, the compound is

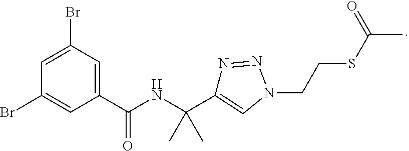 [SMILES: CC(=O)SCCn1cc(C(C)(C)NC(=O)c2cc(Br)cc(Br)c2)nn1]

In embodiments, the compound is

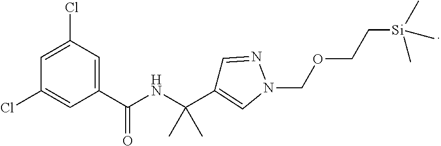 [SMILES: CC(C)(NC(=O)c1cc(Cl)cc(Cl)c1)c1cnn(COCC[Si](C)(C)C)c1]

In embodiments, the compound is

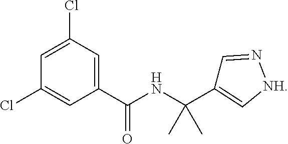 [SMILES: CC(C)(NC(=O)c1cc(Cl)cc(Cl)c1)c1cn[nH]c1]

In embodiments, the compound is

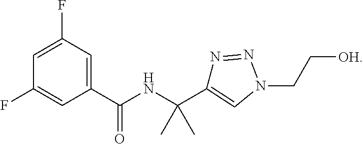 [SMILES: CC(C)(NC(=O)c1cc(F)cc(F)c1)c1cn(CCO)nn1]

In embodiments, the compound is

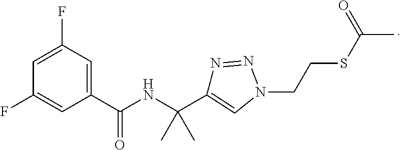 [SMILES: CC(=O)SCCn1cc(C(C)(C)NC(=O)c2cc(F)cc(F)c2)nn1]

In embodiments, the compound is

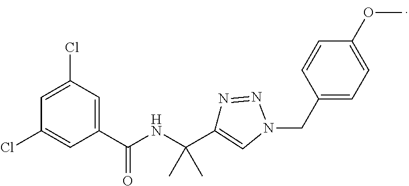 [SMILES: COc1ccc(Cn2cc(C(C)(C)NC(=O)c3cc(Cl)cc(Cl)c3)nn2)cc1]

In embodiments, the compound is

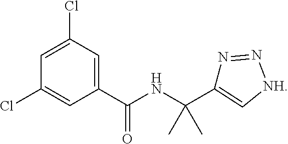 [SMILES: CC(C)(NC(=O)c1cc(Cl)cc(Cl)c1)c1c[nH]nn1]

In embodiments, the compound is

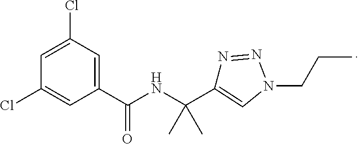 [SMILES: CCCn1cc(C(C)(C)NC(=O)c2cc(Cl)cc(Cl)c2)nn1]

In embodiments, the compound is

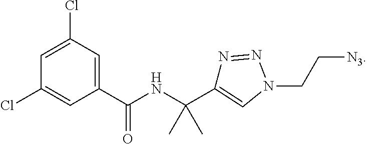 [SMILES: CC(C)(NC(=O)c1cc(Cl)cc(Cl)c1)c1cn(CCN=[N+]=[N-])nn1]

In embodiments, the compound is

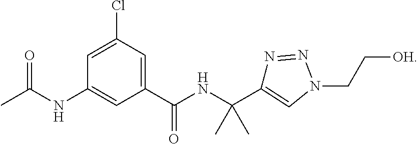 [SMILES: CC(=O)Nc1cc(Cl)cc(C(=O)NC(C)(C)c2cn(CCO)nn2)c1]

In embodiments, the compound is

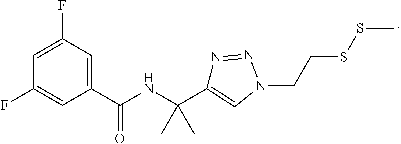 [SMILES: CSSCCn1cc(C(C)(C)NC(=O)c2cc(F)cc(F)c2)nn1]

In embodiments, the compound is

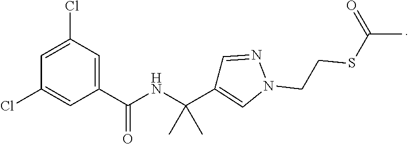 [SMILES: CC(=O)SCCn1cc(C(C)(C)NC(=O)c2cc(Cl)cc(Cl)c2)cn1]

In embodiments, the compound is

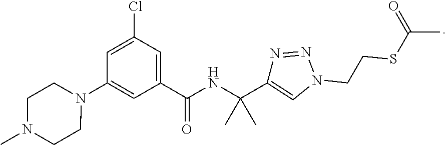 [SMILES: CC(=O)SCCn1cc(C(C)(C)NC(=O)c2cc(Cl)cc(N3CCN(C)CC3)c2)nn1]

In embodiments, the compound is

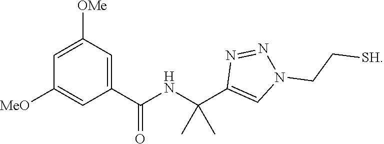 [SMILES: COc1cc(OC)cc(C(=O)NC(C)(C)c2cn(CCS)nn2)c1]

In embodiments, the compound is

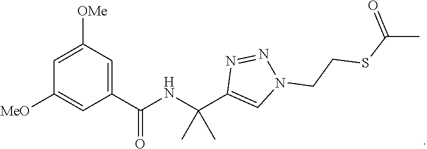 [SMILES: COc1cc(OC)cc(C(=O)NC(C)(C)c2cn(CCSC(C)=O)nn2)c1]

In embodiments, the compound is

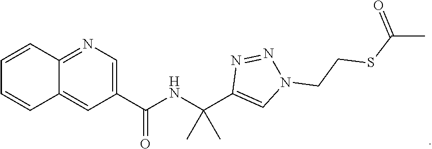 [SMILES: CC(=O)SCCn1cc(C(C)(C)NC(=O)c2cnc3ccccc3c2)nn1]

In embodiments, the compound is

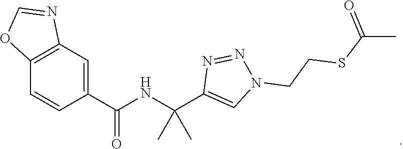 [SMILES: CC(=O)SCCn1cc(C(C)(C)NC(=O)c2ccc3ocnc3c2)nn1]

In embodiments, the compound is

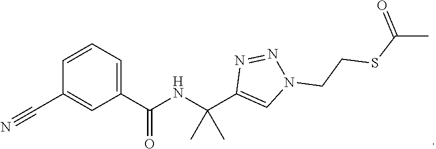 [SMILES: CC(=O)SCCn1cc(C(C)(C)NC(=O)c2cccc(C#N)c2)nn1]

In embodiments, the compound is

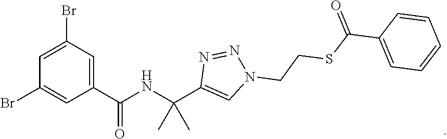 [SMILES: CC(C)(NC(=O)c1cc(Br)cc(Br)c1)c1cn(CCSC(=O)c2ccccc2)nn1]

In embodiments, the compound is

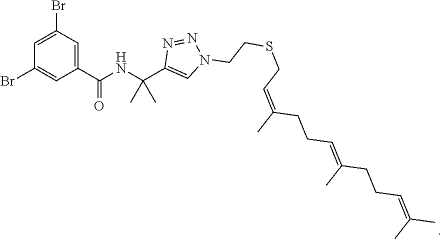 [SMILES: CC(C)=CCC/C(C)=C/CC/C(C)=C\CSCCn1cc(C(C)(C)NC(=O)c2cc(Br)cc(Br)c2)nn1]

In embodiments, the compound is

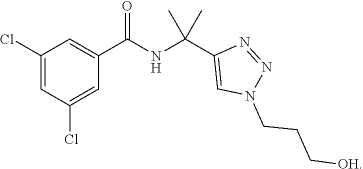 [SMILES: CC(C)(NC(=O)c1cc(Cl)cc(Cl)c1)c1cn(CCCO)nn1]

In embodiments, the compound

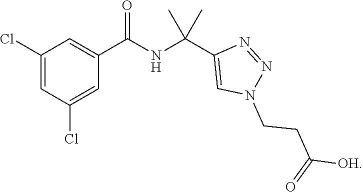 [SMILES: CC(C)(NC(=O)c1cc(Cl)cc(Cl)c1)c1cn(CCC(=O)O)nn1]

In embodiments, the compound is

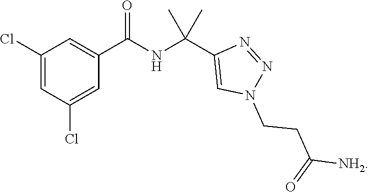 [SMILES: CC(C)(NC(=O)c1cc(Cl)cc(Cl)c1)c1cn(CCC(N)=O)nn1]

In embodiments, the compound is

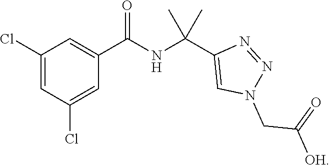 [SMILES: CC(C)(NC(=O)c1cc(Cl)cc(Cl)c1)c1cn(CC(=O)O)nn1]

In embodiments, the compound is

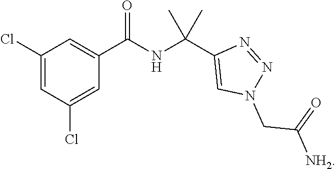 [SMILES: CC(C)(NC(=O)c1cc(Cl)cc(Cl)c1)c1cn(CC(N)=O)nn1]

In embodiments, the compound is

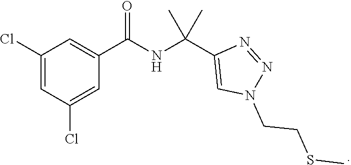 [SMILES: CSCCn1cc(C(C)(C)NC(=O)c2cc(Cl)cc(Cl)c2)nn1]

In embodiments, the compound is

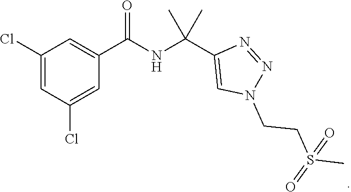 [SMILES: CC(C)(NC(=O)c1cc(Cl)cc(Cl)c1)c1cn(CCS(C)(=O)=O)nn1]

In embodiments, the compound is

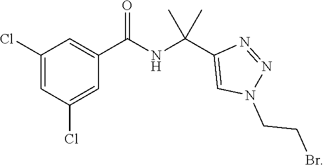 [SMILES: CC(C)(NC(=O)c1cc(Cl)cc(Cl)c1)c1cn(CCBr)nn1]

In embodiments, the compound is

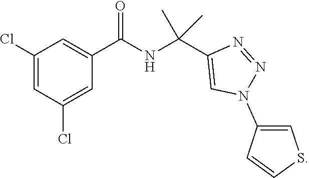 [SMILES: CC(C)(NC(=O)c1cc(Cl)cc(Cl)c1)c1cn(-c2ccsc2)nn1]

In embodiments, the compound is

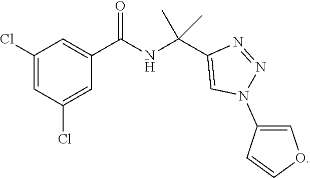 [SMILES: CC(C)(NC(=O)c1cc(Cl)cc(Cl)c1)c1cn(-c2ccoc2)nn1]

In embodiments, the compound is

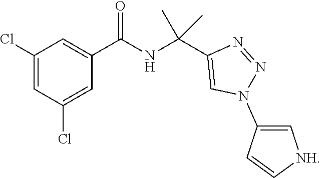 [SMILES: CC(C)(NC(=O)c1cc(Cl)cc(Cl)c1)c1cn(-c2cc[nH]c2)nn1]

In embodiments, the compound is

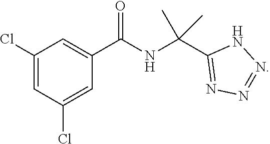 [SMILES: CC(C)(NC(=O)c1cc(Cl)cc(Cl)c1)c1nnn[nH]1]

In embodiments, the compound is

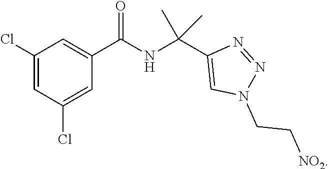 [SMILES: CC(C)(NC(=O)c1cc(Cl)cc(Cl)c1)c1cn(CC[N+](=O)[O-])nn1]

In embodiments, the compound is

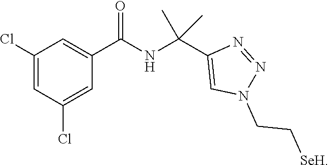

In embodiments, the compound is

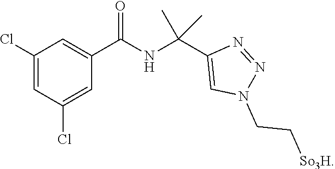

In embodiments, the compound is

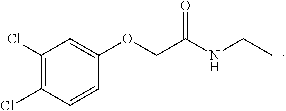

In embodiments, the compound is

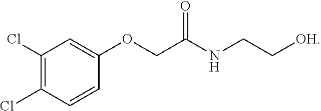 [SMILES: O=C(COc1ccc(Cl)c(Cl)c1)NCCO]

In embodiments, the compound is

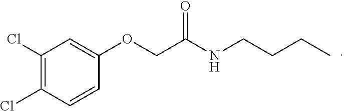 [SMILES: CCCCNC(=O)COc1ccc(Cl)c(Cl)c1]

In embodiments, the compound is

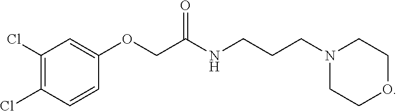 [SMILES: O=C(COc1ccc(Cl)c(Cl)c1)NCCCN1CCOCC1]

In embodiments, the compound is

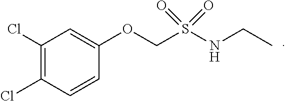 [SMILES: CCNS(=O)(=O)COc1ccc(Cl)c(Cl)c1]

In embodiments, the compound is

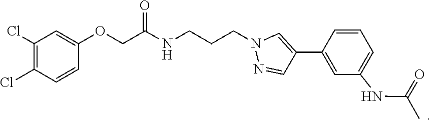 [SMILES: CC(=O)Nc1cccc(-c2cnn(CCCNC(=O)COc3ccc(Cl)c(Cl)c3)c2)c1]

In embodiments, the compound is

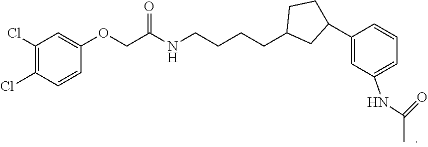 [SMILES: CC(=O)Nc1cccc(C2CCC(CCCCNC(=O)COc3ccc(Cl)c(Cl)c3)C2)c1]

In embodiments, the compound is

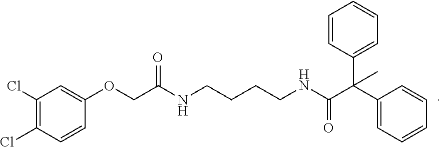 [SMILES: CC(C(=O)NCCCCNC(=O)COc1ccc(Cl)c(Cl)c1)(c1ccccc1)c1ccccc1]

In embodiments, the compound is

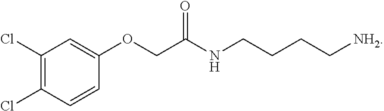 [SMILES: NCCCCNC(=O)COc1ccc(Cl)c(Cl)c1]

In embodiments, the compound is

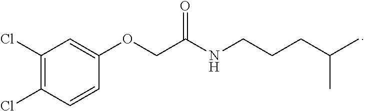 [SMILES: CC(C)CCCNC(=O)COc1ccc(Cl)c(Cl)c1]

In embodiments, the compound is

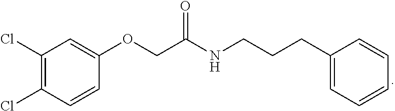 [SMILES: O=C(COc1ccc(Cl)c(Cl)c1)NCCCc1ccccc1]

In embodiments, the compound is

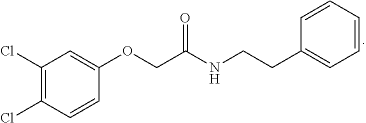 [SMILES: O=C(COc1ccc(Cl)c(Cl)c1)NCCc1ccccc1]

In embodiments, the compound is

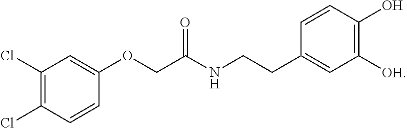 [SMILES: O=C(COc1ccc(Cl)c(Cl)c1)NCCc1ccc(O)c(O)c1]

In embodiments, the compound is

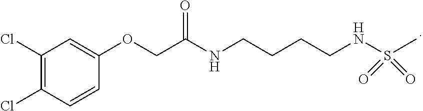 [SMILES: CS(=O)(=O)NCCCCNC(=O)COc1ccc(Cl)c(Cl)c1]

In embodiments, the compound is

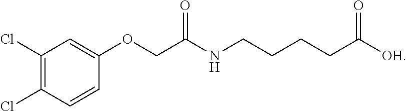 [SMILES: O=C(O)CCCCNC(=O)COc1ccc(Cl)c(Cl)c1]

In embodiments, the compound is

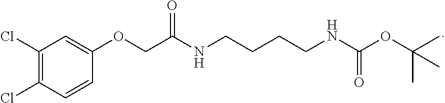 [SMILES: CC(C)(C)OC(=O)NCCCCNC(=O)COc1ccc(Cl)c(Cl)c1]

In embodiments, the compound is

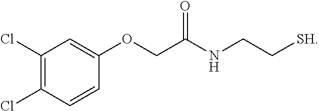 [SMILES: O=C(COc1ccc(Cl)c(Cl)c1)NCCS]

In embodiments, the compound is

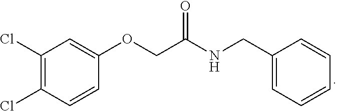 [SMILES: O=C(COc1ccc(Cl)c(Cl)c1)NCc1ccccc1]

In embodiments, the compound is

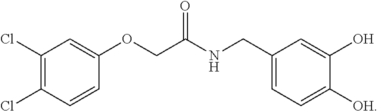 [SMILES: O=C(COc1ccc(Cl)c(Cl)c1)NCc1ccc(O)c(O)c1]

In embodiments, the compound is

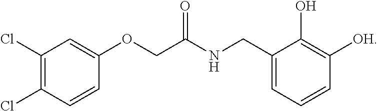 [SMILES: O=C(COc1ccc(Cl)c(Cl)c1)NCc1cccc(O)c1O]

In embodiments, the compound is

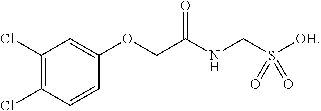 [SMILES: O=C(COc1ccc(Cl)c(Cl)c1)NCS(=O)(=O)O]

In embodiments, the compound is

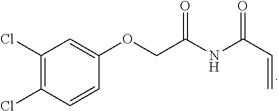 [SMILES: C=CC(=O)NC(=O)COc1ccc(Cl)c(Cl)c1]

In embodiments, the compound is

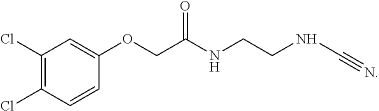 [SMILES: N#CNCCNC(=O)COc1ccc(Cl)c(Cl)c1]

In embodiments, the compound is

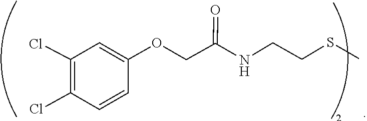

In embodiments, the compound is

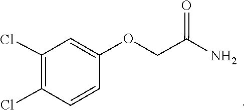 [SMILES: NC(=O)COc1ccc(Cl)c(Cl)c1]

In embodiments, the compound is

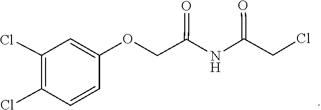 [SMILES: O=C(CCl)NC(=O)COc1ccc(Cl)c(Cl)c1]

In embodiments, the compound is

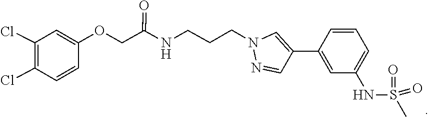 [SMILES: CS(=O)(=O)Nc1cccc(-c2cnn(CCCNC(=O)COc3ccc(Cl)c(Cl)c3)c2)c1]

In embodiments, the compound is

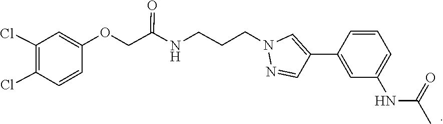 [SMILES: CC(=O)Nc1cccc(-c2cnn(CCCNC(=O)COc3ccc(Cl)c(Cl)c3)c2)c1]

In embodiments, the compound is

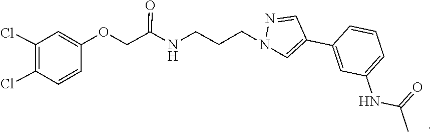 [SMILES: CC(=O)Nc1cccc(-c2cnn(CCCNC(=O)COc3ccc(Cl)c(Cl)c3)c2)c1]

In embodiments, the compound is

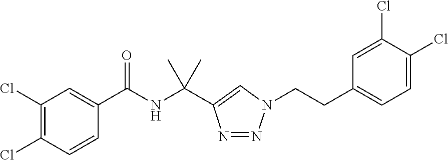 [SMILES: CC(C)(NC(=O)c1ccc(Cl)c(Cl)c1)c1cn(CCc2ccc(Cl)c(Cl)c2)nn1]

In embodiments, the compound is

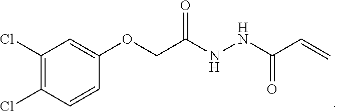 [SMILES: C=CC(=O)NNC(=O)COc1ccc(Cl)c(Cl)c1]

In embodiments, the compound is

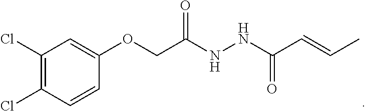 [SMILES: C/C=C/C(=O)NNC(=O)COc1ccc(Cl)c(Cl)c1]

In embodiments, the compound is

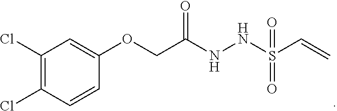 [SMILES: C=CS(=O)(=O)NNC(=O)COc1ccc(Cl)c(Cl)c1]

In embodiments, the compound is

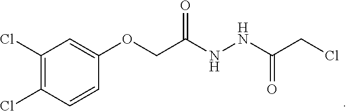 [SMILES: O=C(CCl)NNC(=O)COc1ccc(Cl)c(Cl)c1]

In embodiments, the compound is

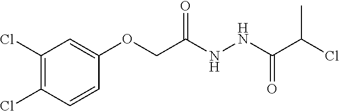 [SMILES: CC(Cl)C(=O)NNC(=O)COc1ccc(Cl)c(Cl)c1]

In embodiments, Ring A is not

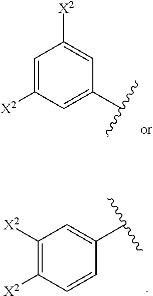

In embodiments, Ring A is not

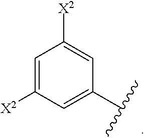

In embodiments, Ring A is not

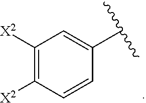

X² is independently —F, —Cl, —Br, or —I. In embodiments, X² is independently —Cl.

In embodiments, R¹ is not —SSR^{1D}. R^{1D} is as described herein.

In embodiments, E is not —SS-(unsubstituted C₁-C₇ alkyl). In embodiments, E is not —SS-(3 to 7 membered unsubstituted heteroalkyl. In embodiments, E is not —S SCH₂CH₂N(CH₃)₂.

In embodiments, the compound is not

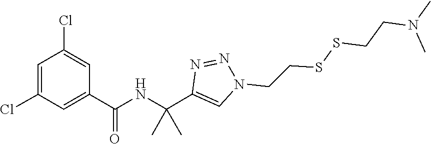 [SMILES: CN(C)CCSSCCn1cc(C(C)(C)NC(=O)c2cc(Cl)cc(Cl)c2)nn1]

or

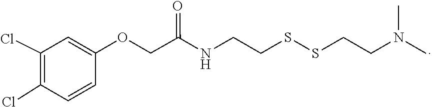 [SMILES: CN(C)CCSSCCNC(=O)COc1ccc(Cl)c(Cl)c1]

In embodiments, the compound is not

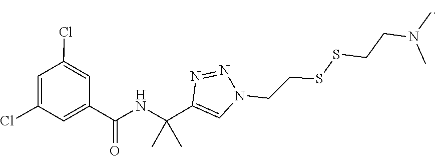 [SMILES: CN(C)CCSSCCn1cc(C(C)(C)NC(=O)c2cc(Cl)cc(Cl)c2)nn1]

In embodiments, the compound is not

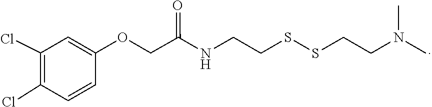 [SMILES: CN(C)CCSSCCNC(=O)COc1ccc(Cl)c(Cl)c1]

In embodiments, the compound covalently binds Nurr1 (e.g., human Nurr1). In embodiments, the compound irreversibly covalently binds Nurr1 (e.g., human Nurr1). In embodiments, the compound reversibly covalently binds Nurr1 (e.g., human Nurr1).

In embodiments, the compound contacts an amino acid corresponding to Cys566 of human Nurr1. In embodiments, the compound contacts an amino acid corresponding to Cys475 of human Nurr1. In embodiments, the compound contacts an amino acid corresponding to Cys534 of human Nurr1.

In embodiments, the compound contacts an amino acid corresponding to Arg515 of human Nurr1. In embodiments, the compound contacts an amino acid corresponding to Arg563 of human Nurr1. In embodiments, the compound contacts an amino acid corresponding to Glu445 of human Nurr1.

In embodiments, the compound covalently binds an amino acid corresponding to Cys566 of human Nurr1. In embodiments, the compound irreversibly covalently binds an amino acid corresponding to Cys566 of human Nurr1. In embodiments, the compound reversibly covalently binds an amino acid corresponding to Cys566 of human Nurr1.

In embodiments, the compound stabilizes a Nurr1 monomer. In embodiments, the compound stabilizes a Nurr1 homodimer. In embodiments, the compound stabilizes a head-to-tail Nurr1 homodimer. In embodiments, the compound stabilizes a Nurr1 heterodimer. In embodiments, the Nurr1 heterodimer is a heterodimer with RXRα.

In embodiments, the compound stabilizes a Nurr1 monomer relative to a control (e.g., absence of the compound). In embodiments, the compound stabilizes a Nurr1 homodimer relative to a control (e.g., absence of the compound). In embodiments, the compound stabilizes a head-to-tail Nurr1 homodimer relative to a control (e.g., absence of the compound). In embodiments, the compound stabilizes a Nurr1 heterodimer relative to a control (e.g., absence of the compound). In embodiments, the Nurr1 heterodimer is a heterodimer with RXRα.

In embodiments, the compound contacts a Nurr1 monomer. In embodiments, the compound contacts a Nurr1 homodimer. In embodiments, the compound contacts a head-to-tail Nurr1 homodimer. In embodiments, the compound contacts a Nurr1 heterodimer. In embodiments, the Nurr1 heterodimer is a heterodimer with RXRα.

In embodiments, the compound binds a Nurr1 monomer. In embodiments, the compound binds a Nurr1 homodimer. In embodiments, the compound binds a head-to-tail Nurr1 homodimer. In embodiments, the compound binds a Nurr1 heterodimer. In embodiments, the Nurr1 heterodimer is a heterodimer with RXRα.

In embodiments, the compound precludes the formation of Nurr1:RXR heterodimers. In embodiments, the compound inhibits the formation of Nurr1:RXR heterodimers. In embodiments, compound binding to Nurr1 inhibits the resulting compound:Nurr1 complex from binding to RXR.

In embodiments, the compound stabilizes a Nurr1 dimer conformation wherein the distance between the N-termini is about 74.0 Å (e.g., about 60, 61, 62, 63, 64, 65, 66, 67, 68, 69, 70, 71, 72, 73, 75, 76, 77, 78, 79, 80, 81, 82, 83, 84, 85, 86, 87, 88, 89, or 90 Å). In embodiments, the compound stabilizes a Nurr1 dimer conformation wherein the distance between the N-termini is at least 74.0 Å (e.g., at least 75, 76, 77, 78, 79, 80, 81, 82, 83, 84, 85, 86, 87, 88, 89, or 90 Å). In embodiments, the compound stabilizes a Nurr1 dimer conformation wherein the distance between the N-termini is less than 74.0 Å (e.g., less than 73, 72, 71, 70, 69, 68, 67, 66, 65, 65, 64, 63, 62, 61, or 60 Å).

In embodiments, the compound contacts a Nurr1 dimer conformation wherein the distance between the N-termini is about 74.0 Å (e.g., about 60, 61, 62, 63, 64, 65, 66, 67, 68, 69, 70, 71, 72, 73, 75, 76, 77, 78, 79, 80, 81, 82, 83, 84, 85, 86, 87, 88, 89, or 90 Å). In embodiments, the compound contacts a Nurr1 dimer conformation wherein the distance between the N-termini is at least 74.0 Å (e.g., at least 75, 76, 77, 78, 79, 80, 81, 82, 83, 84, 85, 86, 87, 88, 89, or 90 Å). In embodiments, the compound contacts a Nurr1 dimer conformation wherein the distance between the N-termini is less than 74.0 Å (e.g., less than 73, 72, 71, 70, 69, 68, 67, 66, 65, 65, 64, 63, 62, 61, or 60 Å).

In embodiments, the compound binds a Nurr1 dimer conformation wherein the distance between the N-termini is about 74.0 Å (e.g., about 60, 61, 62, 63, 64, 65, 66, 67, 68, 69, 70, 71, 72, 73, 75, 76, 77, 78, 79, 80, 81, 82, 83, 84, 85, 86, 87, 88, 89, or 90 Å). In embodiments, the compound binds a Nurr1 dimer conformation wherein the distance between the N-termini is at least 74.0 Å (e.g., at least 75, 76, 77, 78, 79, 80, 81, 82, 83, 84, 85, 86, 87, 88, 89, or 90 Å). In embodiments, the compound binds a Nurr1 dimer conformation wherein the distance between the N-termini is less than 74.0 Å (e.g., less than 73, 72, 71, 70, 69, 68, 67, 66, 65, 65, 64, 63, 62, 61, or 60 Å).

In embodiments, the compound stabilizes a Nurr1 dimer conformation wherein the distance between the N-termini is about 59.3 Å (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 Å). In embodiments, the compound stabilizes a Nurr1 dimer conformation wherein the distance between the N-termini is at least 59.3 Å (e.g., at least 60, 61, 62, 63, 64, 65, 66, 67, 68, 69, 70, 71, 72, 73, 74, or 75 Å). In embodiments, the compound stabilizes a Nurr1 dimer conformation wherein the distance between the N-termini is less than 59.3 Å (e.g., less than 59, 58, 57, 56, 55, 54, 53, 52, 51, 50, 49, 48, 47, 46, 45, 44, 43, 42, 41, or 40 Å).

In embodiments, the compound contacts a Nurr1 dimer conformation wherein the distance between the N-termini is about 59.3 Å (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 Å). In embodiments, the compound contacts a Nurr1 dimer conformation wherein the distance between the N-termini is at least 59.3 Å (e.g., at least 60, 61, 62, 63, 64, 65, 66, 67, 68, 69, 70, 71, 72, 73, 74, or 75 Å). In embodiments, the compound contacts a Nurr1 dimer conformation wherein the distance between the N-termini is less than 59.3 Å (e.g., less than 59, 58, 57, 56, 55, 54, 53, 52, 51, 50, 49, 48, 47, 46, 45, 44, 43, 42, 41, or 40 Å).

In embodiments, the compound binds a Nurr1 dimer conformation wherein the distance between the N-termini is about 59.3 Å (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 Å). In embodiments, the compound binds a Nurr1 dimer conformation wherein the distance between the N-termini is at least 59.3 Å (e.g., at least 60, 61, 62, 63, 64, 65, 66, 67, 68, 69, 70, 71, 72, 73, 74, or 75 Å). In embodiments, the compound binds a Nurr1 dimer conformation wherein the distance between the N-termini is less than 59.3 Å (e.g., less than 59, 58, 57, 56, 55, 54, 53, 52, 51, 50, 49, 48, 47, 46, 45, 44, 43, 42, 41, or 40 Å).

In embodiments, the compound binds Nurr1 and induces Nurr1 binding to a NBRE, a NuRE, or a DR-5 response element. In embodiments, the compound binds Nurr1 and induces Nurr1 binding to a NBRE. In embodiments, the compound binds Nurr1 and induces Nurr1 binding to a NuRE. In embodiments, the compound binds Nurr1 and induces Nurr1 binding to a DR-5 response element.

In embodiments, the compound is a compound as described herein, including in embodiments. In embodiments the compound is a compound described herein (e.g., in the examples section, in the figures, in the tables, in the claims, or in the appendix).

III. Pharmaceutical compositions

In an aspect is provided a pharmaceutical composition including a compound described herein and a pharmaceutically acceptable excipient.

In embodiments, the pharmaceutical composition includes an effective amount of the compound. In embodiments, the pharmaceutical composition includes a therapeutically effective amount of the compound.

In embodiments, the pharmaceutical composition includes an effective amount of a second agent, wherein the second agent is an agent for treating a neurodegenerative disease. In embodiments, the neurodegenerative disease is Parkinson's disease. In embodiments, the second agent is a Parkinson's disease drug, for example, levodopa, carbidopa, selegiline, amantadine, donepezil, galanthamine, rivastigmine, tacrine, bromocriptine, pergolide, pramipexole, ropinirole, trihexyphenidyl, benztropine, biperiden, procyclidine, tolcapone, or entacapone. In embodiments, the pharmaceutical composition includes a therapeutically effective amount of the second agent.

In embodiments, the pharmaceutical composition includes an effective amount of a second agent, wherein the second agent is an agent for treating an inflammatory disease, for example, acetaminophen, duloxetine, aspirin, ibuprofen, naproxen, diclofenac, prednisone, betamethasone, cortisone, dexamethasone, hydrocortisone, methylprednisolone, prednisolone, codeine, fentanyl, hydrocodone, hydromorphone, morphine, meperidine, or oxycodone. In embodiments, the pharmaceutical composition includes a therapeutically effective amount of the second agent.

In embodiments, the pharmaceutical composition includes an effective amount of a second agent, wherein the second agent is an anti-cancer agent.

In an aspect is provided a pharmaceutical composition including 5,6-dihydroxyindole (DHI) and a pharmaceutically acceptable excipient. In embodiments, the pharmaceutical composition includes an effective amount of 5,6-dihydroxyindole (DHI). In embodiments, the pharmaceutical composition includes a therapeutically effective amount of 5,6-dihydroxyindole (DHI). In embodiments, the pharmaceutical composition includes an effective amount of a second agent described herein.

IV. Methods of Use

In an aspect is provided a method for treating a disease associated with dysregulation and/or degeneration of dopaminergic neurons in the central nervous system of a subject in need thereof, the method including administering to the subject in need thereof a therapeutically effective amount of a compound described herein.

In an aspect is provided a method for treating a disease associated with dysregulation and/or degeneration of dopaminergic neurons in the central nervous system of a subject in need thereof, the method including administering to the subject in need thereof a therapeutically effective amount of 5,6-dihydroxyindole (DHI).

In embodiments, the disease associated with dysregulation and/or degeneration of dopaminergic neurons is Parkinson's disease, Alzheimer's disease, multiple sclerosis, amyotrophic lateral sclerosis, schizophrenia, or drug addiction. In embodiments, the disease is Parkinson's disease. In embodiments, the disease is Alzheimer's disease. In embodiments, the disease is multiple sclerosis. In embodiments, the disease is amyotrophic lateral sclerosis. In embodiments, the disease is schizophrenia. In embodiments, the disease is drug addiction.

In embodiments, the disease associated with dysregulation and/or degeneration of dopaminergic neurons is a cancer.

In an aspect is provided a method for treating a disease in a subject in need thereof, the method including administering to the subject in need thereof a therapeutically effective amount of a compound described herein.

In an aspect is provided a method for treating a disease in a subject in need thereof, the method including administering to the subject in need thereof a therapeutically effective amount of 5,6-dihydroxyindole (DHI).

In embodiments, the disease is Parkinson's disease, Alzheimer's disease, multiple sclerosis, amyotrophic lateral sclerosis, schizophrenia, or drug addiction. In embodiments, the disease is Parkinson's disease. In embodiments, the disease is Alzheimer's disease. In embodiments, the disease is multiple sclerosis. In embodiments, the disease is amyotrophic lateral sclerosis. In embodiments, the disease is schizophrenia. In embodiments, the disease is drug addiction.

In embodiments, the disease is a cancer.

In embodiments, the cancer is breast cancer, pancreatic cancer, bladder cancer, mucoepidermoid carcinoma, gastric cancer, prostate cancer, colorectal cancer, lung cancer, adrenocortical cancer, or cervical cancer.

In an aspect is provided a method for reducing inflammation in a subject in need thereof, the method including administering to the subject in need thereof a therapeutically effective amount of a compound described herein.

In embodiments, the method is for reducing inflammation in the central nervous sytem of the subject in need thereof.

In an aspect is provided a method for reducing oxidative stress in a subject in need thereof, the method including administering to the subject in need thereof a therapeutically effective amount of a compound described herein.

In embodiments, the method is for reducting oxidative stress in the central nervous system of the subject in need thereof.

In an aspect is provided a method of modulating the level of activity of Nurr1 in a subject in need thereof, the method including administering to the subject in need thereof a therapeutically effective amount of a compound described herein. In embodiments, the level of activity of Nurr1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Nurr1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of Nurr1 in a cell, the method including contacting the cell with a compound described herein. In embodiments, the level of activity of Nurr1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Nurr1 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of Pitx3 in a subject in need thereof, the method including administering to the subject in need thereof a therapeutically effective amount of a compound described herein. In embodiments, the level of activity of Pitx3 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of Pitx3 in a cell, the method including contacting the cell with a compound described herein. In embodiments, the level of activity of Pitx3 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of TH in a subject in need thereof, the method including administering to the subject in need thereof a therapeutically effective amount of a compound described herein. In embodiments, the level of activity of TH in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of TH in a cell, the method including contacting the cell with a compound described herein. In embodiments, the level of activity of TH in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of VMAT2 in a subject in need thereof, the method including administering to the subject in need thereof a therapeutically effective amount of a compound described herein. In embodiments, the level of activity of VMAT2 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of VMAT2 in a cell, the method including contacting the cell with a compound described herein. In embodiments, the level of activity of VMAT2 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of dopa decarboxylase (DDC) in a subject in need thereof, the method including administering to the subject in need thereof a therapeutically effective amount of a compound described herein. In embodiments, the level of activity of DDC in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of dopa decarboxylase (DDC) in a cell, the method including contacting the cell with a compound described herein. In embodiments, the level of activity of DDC in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of dopamine transporter (DAT) in a subject in need thereof, the method including administering to the subject in need thereof a therapeutically effective amount of a compound described herein. In embodiments, the level of activity of DAT in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of dopamine transporter (DAT) in a cell, the method including contacting the cell with a compound described herein. In embodiments, the level of activity of DAT in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of BDNF in a subject in need thereof, the method including administering to the subject in need thereof a therapeutically effective amount of a compound described herein. In embodiments, the level of activity of BDNF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of BDNF in a cell, the method including contacting the cell with a compound described herein. In embodiments, the level of activity of BDNF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of NGF in a subject in need thereof, the method including administering to the subject in need thereof a therapeutically effective amount of a compound described herein. In embodiments, the level of activity of NGF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of NGF in a cell, the method including contacting the cell with a compound described herein. In embodiments, the level of activity of NGF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of GDNF receptor c-Ret in a subject in need thereof, the method including administering to the subject in need thereof a therapeutically effective amount of a compound described herein. In embodiments, the level of activity of GDNF receptor c-Ret in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of GDNF receptor c-Ret in a cell, the method including contacting the cell with a compound described herein. In embodiments, the level of activity of GDNF receptor c-Ret in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of SOD1 in a subject in need thereof, the method including administering to the subject in need thereof a therapeutically effective amount of a compound described herein. In embodiments, the level of activity of SOD1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of increasing the level of activity of SOD1 in a cell, the method including contacting the cell with a compound described herein. In embodiments, the level of activity of SOD1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of TNFα in a subject in need thereof, the method including administering to the subject in need thereof a therapeutically effective amount of a compound described herein. In embodiments, the level of activity of TNFα in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of TNFα in a cell, the method including contacting the cell with a compound described herein. In embodiments, the level of activity of TNFα in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of iNOS in a subject in need thereof, the method including administering to the subject in need thereof a therapeutically effective amount of a compound described herein. In embodiments, the level of activity of iNOS in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of iNOS in a cell, the method including contacting the cell with a compound described herein. In embodiments, the level of activity of iNOS in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of IL1-β in a subject in need thereof, the method including administering to the subject in need thereof a therapeutically effective amount of a compound described herein. In embodiments, the level of activity of IL-1β in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1β in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In an aspect is provided a method of reducing the level of activity of IL-1β in a cell, the method including contacting the cell with a compound described herein. In embodiments, the level of activity of IL-1β in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1β in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing the level of dopamine in a subject in need thereof, the method including administering to the subject in need thereof a therapeutically effective amount of a compound described herein. In embodiments, the level of dopamine in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of dopamine in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing the level of dopamine in a cell, the method including contacting the cell with a compound described herein. In embodiments, the level of dopamine in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of dopamine in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing synthesis of dopamine in a cell with a compound described herein as compared to a control (e.g., absence of the compound). In embodiments, the level of synthesis of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of synthesis of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing packaging of dopamine in a cell with a compound described herein as compared to a control (e.g., absence of the compound). In embodiments, the level of packaging of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of packaging of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing reuptake of dopamine in a cell with a compound described herein as compared to a control (e.g., absence of the compound). In embodiments, the level of reuptake of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of reuptake of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing development of dopaminergic neurons with a compound described herein as compared to a control (e.g., absence of the compound). In embodiments, the level of development of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of development of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing maintenance of dopaminergic neurons with a compound described herein as compared to a control (e.g., absence of the compound). In embodiments, the level of maintenance of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of maintenance of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing survival of dopaminergic neurons with a compound described herein as compared to a control (e.g., absence of the compound). In embodiments, the level of survival of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of survival of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes covalently binding Nurr1 (e.g., human Nurr1) with a compound described herein. In embodiments, the method includes irreversibly covalently binding Nurr1 (e.g., human Nurr1) with a compound described herein. In embodiments, the method includes reversibly covalently binding Nurr1 (e.g., human Nurr1) with a compound described herein.

In embodiments, the method includes contacting an amino acid corresponding to Cys566 of human Nurr1 with a compound described herein. In embodiments, the method includes contacting an amino acid corresponding to Cys475 of human Nurr1 with a compound described herein. In embodiments, the method includes contacting an amino acid corresponding to Cys534 of human Nurr1 with a compound described herein.

In embodiments, the method includes contacting an amino acid corresponding to Arg515 of human Nurr1 with a compound described herein. In embodiments, the method includes contacting an amino acid corresponding to Arg563 of human Nurr1 with a compound described herein. In embodiments, the method includes contacting an amino acid corresponding to Glu445 of human Nurr1 with a compound described herein.

In embodiments, the method includes covalently binding an amino acid corresponding to Cys566 of human Nurr1 with a compound described herein. In embodiments, the method includes irreversibly covalently binding an amino acid corresponding to Cys566 of human Nurr1 with a compound described herein. In embodiments, the method includes reversibly covalently binding an amino acid corresponding to Cys566 of human Nurr1 with a compound described herein.

In embodiments, the method includes stabilizing a Nurr1 monomer with a compound described herein. In embodiments, the method includes stabilizing a Nurr1 homodimer with a compound described herein. In embodiments, the method includes stabilizing a head-to-tail Nurr1 homodimer with a compound described herein. In embodiments, the method includes stabilizing a Nurr1 heterodimer with a compound described herein. In embodiments, the Nurr1 heterodimer is a heterodimer with RXRα.

In embodiments, the method includes contacting a Nurr1 monomer with a compound described herein. In embodiments, the method includes contacting a Nurr1 homodimer with a compound described herein. In embodiments, the method includes contacting a head-to-tail Nurr1 homodimer with a compound described herein. In embodiments, the method includes contacting a Nurr1 heterodimer with a compound described herein. In embodiments, the Nurr1 heterodimer is a heterodimer with RXRα.

In embodiments, the method includes binding a Nurr1 monomer with a compound described herein. In embodiments, the method includes binding a Nurr1 homodimer with a compound described herein. In embodiments, the method includes binding a head-to-tail Nurr1 homodimer with a compound described herein. In embodiments, the method includes binding a Nurr1 heterodimer with a compound described herein. In embodiments, the Nurr1 heterodimer is a heterodimer with RXRα.

In embodiments, the method includes precluding the formation of Nurr1:RXR heterodimers with a compound described herein.

In embodiments, the method includes stabilizing a Nurr1 dimer conformation wherein the distance between the N-termini is about 74.0 Å with a compound described herein. In embodiments, the method includes stabilizing a Nurr1 dimer conformation wherein the distance between the N-termini is at least 74.0 Å with a compound described herein. In embodiments, the method includes stabilizing a Nurr1 dimer conformation wherein the distance between the N-termini is less than 74.0 Å with a compound described herein.

In embodiments, the method includes contacting a Nurr1 dimer conformation wherein the distance between the N-termini is about 74.0 Å with a compound described herein. In embodiments, the method includes contacting a Nurr1 dimer conformation wherein the distance between the N-termini is at least 74.0 Å with a compound described herein. In embodiments, the method includes contacting a Nurr1 dimer conformation wherein the distance between the N-termini is less than 74.0 Å with a compound described herein.

In embodiments, the method includes binding a Nurr1 dimer conformation wherein the distance between the N-termini is about 74.0 Å with a compound described herein. In embodiments, the method includes binding a Nurr1 dimer conformation wherein the distance between the N-termini is at least 74.0 Å with a compound described herein. In embodiments, the method includes binding a Nurr1 dimer conformation wherein the distance between the N-termini is less than 74.0 Å with a compound described herein.

In embodiments, the method includes stabilizing a Nurr1 dimer conformation wherein the distance between the N-termini is about 59.3 Å with a compound described herein. In embodiments, the method includes stabilizing a Nurr1 dimer conformation wherein the distance between the N-termini is at least 59.3 Å with a compound described herein. In embodiments, the method includes stabilizing a Nurr1 dimer conformation wherein the distance between the N-termini is less than 59.3 Å with a compound described herein.

In embodiments, the method includes contacting a Nurr1 dimer conformation wherein the distance between the N-termini is about 59.3 Å with a compound described herein. In embodiments, the method includes contacting a Nurr1 dimer conformation wherein the distance between the N-termini is at least 59.3 Å with a compound described herein. In embodiments, the method includes contacting a Nurr1 dimer conformation wherein the distance between the N-termini is less than 59.3 Å with a compound described herein.

In embodiments, the method includes binding a Nurr1 dimer conformation wherein the distance between the N-termini is about 59.3 Å with a compound described herein. In embodiments, the method includes binding a Nurr1 dimer conformation wherein the distance between the N-termini is at least 59.3 Å with a compound described herein. In embodiments, the method includes binding a Nurr1 dimer conformation wherein the distance between the N-termini is less than 59.3 Å with a compound described herein.

In embodiments, the method includes binding a Nurr1 and inducing Nurr1 binding to a NBRE, a NuRE, or a DR-5 response element. In embodiments, the method includes binding a Nurr1 and inducing Nurr1 binding to a NBRE. In embodiments, the method includes binding a Nurr1 and inducing Nurr1 binding to a NuRE. In embodiments, the method includes binding a Nurr1 and inducing Nurr1 binding to a DR-5 response element.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

V. Embodiments

Embodiment P1. A compound having the formula

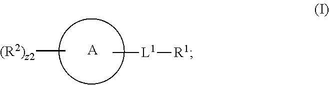

wherein
Ring A is aryl or heteroaryl;
L¹ is L¹⁰¹, L¹⁰²-L¹⁰³,
L¹⁰¹ is a bond, —S(O)₂—, —N(R¹⁰¹)—, —O—, —S—, —C(O)—, —C(O)N(R¹⁰¹)—, —N(R¹⁰¹)C(O)—), —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰¹, R¹⁰² and R¹⁰³ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl;
R¹ is hydrogen, halogen, —CX¹₃, —CHX¹₂, —CH₂X¹, —OCX¹₃, —OCH₂X¹, —OCHX¹₂, —CN —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)OR^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —N₃, —SSR^{1D}, —SiR^{1A}R^{1B}R^{1C}, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}_{R}^{2B}, —C(O)R^{2C}, —C(O)OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1l, and v2 are independently 1 or 2;
X¹ and X² are independently —F, —Cl, —Br, or —I; and
z2 is an integer from 0 to 5.

Embodiment P2. The compound of embodiment P1, wherein the compound has the formula

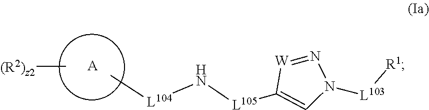

wherein
L¹⁰⁴ is a bond, —S(O)₂—, —C(O)—, —NHC(O)—, —OC(O)—, substituted or unsubstituted alkylene, or substituted or unsubstituted heteroalkylene;
L¹⁰⁵ is a bond, substituted or unsubstituted alkylene, substituted or unsubstituted heteroalkylene, substituted or unsubstituted cycloalkylene, or substituted or unsubstituted heterocycloalkylene;
L¹⁰³ is a bond, substituted or unsubstituted alkylene, or substituted or unsubstituted heteroalkylene; and
W is N or CH.

Embodiment P3. The compound of embodiment P2, wherein Ring A is a phenyl or 5 to 10 membered heteroaryl.

Embodiment P4. The compound of embodiment P2, wherein Ring A is a phenyl.

Embodiment P5. The compound of embodiment P2, wherein Ring A is

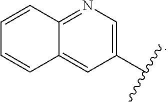 [SMILES: *c1cnc2ccccc2c1]

Embodiment P6. The compound of one of embodiments P2 to P4, wherein the compound has the formula

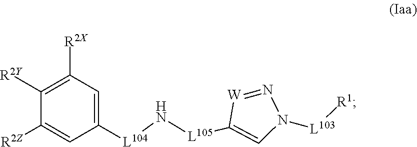

and
R^{2X}, R^{2Y}, and R^{2Z} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)^{m2}, —NR^{2A}R^{2B}, —C(O)R^{2C}, —C(O)OR^{2C}, —C(O)NR^{2A}R^{2B}, —OR^{D2}, —NR^{2A}SO₂R^{2D}, —NR^{2A}C(O)_{R}^{2C}, —NR^{2A}C(O)OR^{2C}, —NR^{2A}OR^{2C},—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2X} and R^{2Y} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl; R^{2Y} and R^{2Z} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

Embodiment P7. The compound of embodiment P6, wherein
R^{2X} is independently halogen or unsubstituted heteroalkyl;
R^{2Y} is independently hydrogen or halogen; and
R^{2Z} is independently hydrogen, halogen, —CN, —NR^{2A}C(O)R^{2C}, unsubstitued heteroalkyl, or substituted or unsubstituted heterocycloalkyl.

Embodiment P8. The compound of embodiment P6, wherein
R^{2X} is independently halogen;
R^{2Y} is independently halogen; and
R^{2Z} is independently hydrogen.

Embodiment P9. The compound of embodiment P6, wherein
R^{2X} is independently halogen or unsubstituted 2 to 4 membered heteroalkyl;
R^{2Y} is independently hydrogen;
R^{2Z} is independently halogen, —CN, —NR^{2A}C(O)R^{2C}, unsubstituted 2 to 4 membered heteroalkyl, or substituted or unsubstituted 5 to 6 membered heterocycloalkyl;
R^{2A} is independently hydrogen; and
R^{2C} is independently unsubstituted C₁-C₂ alkyl.

Embodiment P10. The compound of one of embodiments P2 to P9, wherein L¹⁰⁴ is —C(O)—.

Embodiment P11. The compound of one of embodiments P2 to P10, wherein L¹⁰⁵ is an unsubstituted alkylene.

Embodiment P12. The compound of one of embodiments P2 to P10, wherein L¹⁰⁵ is an unsubstituted C₁-C₄ alkylene.

Embodiment P13. The compound of one of embodiments P2 to P10, wherein L¹⁰⁵ is

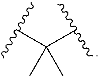 [SMILES: *C(*)(C)C]

Embodiment P14. The compound of one of embodiments P2 to P13, wherein W is N.

Embodiment P15. The compound of one of embodiments P2 to P14, wherein L¹⁰³ is an unsubstituted alkylene.

Embodiment P16. The compound of one of embodiments P2 to P14, wherein L¹⁰³ is an unsubstituted C₁-C₄ alkylene.

Embodiment P17. The compound of one of embodiments P2 to P14, wherein L¹⁰³ is an unsubstituted ethylene.

Embodiment P18. The compound of one of embodiments P2 to P9, wherein

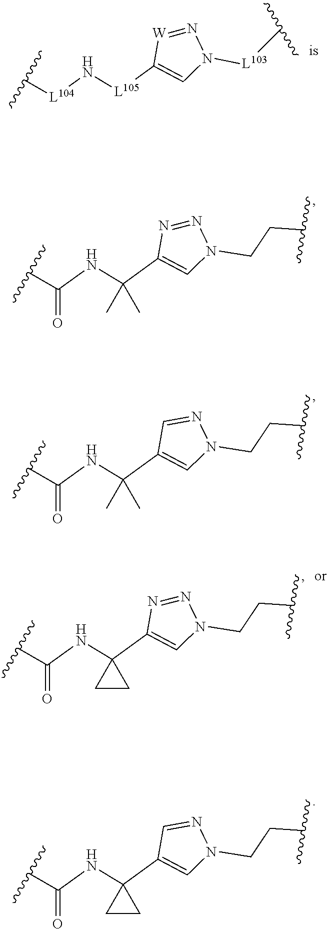

Embodiment P19. The compound of one of embodiments P1 to P18, wherein
R¹ is —SR^{1D}, —NR^{1A}R^{1B}, —OR^{1D}, E, unsubstituted alkyl, substituted or unsubstituted phenyl, or substituted or unsubstituted 5 to 6 membered heteroaryl;
R^{1A} is independently hydrogen or unsubstituted C₁-C₄ alkyl;
R^{1B} is independently hydrogen or unsubstituted C₁-C₄ alkyl; and
R^{1D} is independently hydrogen, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, SO₄H, SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, —N₃, —PO₃H₂, or substituted or unsubstituted alkyl.

Embodiment P20. The compound of one of embodiments P1 to P18, wherein
R¹ is —SR^{1D}, —NR^{1A}R^{1B}, —OR^{1D}, E, unsubstituted C₁-C₄ alkyl, R¹⁰-substituted or unsubstituted phenyl, or R¹⁰-substituted or unsubstituted 5 to 6 membered heteroaryl;
R^{1A} is independently hydrogen or unsubstituted C₁-C₄ alkyl;
R^{1B} is independently hydrogen or unsubstituted C₁-C₄ alkyl;
R^{1D} is independently hydrogen, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, SO₄H, SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, —N₃, —PO₃H₂, R¹⁰-substituted or unsubstituted C₁-C₄ alkyl; and
R¹⁰ is oxo, halogen, —CCl₃, —CF₃, —CI₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment P21. The compound of one of embodiments P1 to P18, wherein R¹ is —SR^{1D} or R¹⁰-substituted phenyl;
R^{1D} is independently hydrogen, halogen, —CCl₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂, —N₃, —PO₃H₂, R¹⁰-substituted or unsubstituted C₁-C₄ alkyl; and
R¹⁰ is oxo, halogen, —CCl₃, —CF₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment P22. The compound of one of embodiments P1 to P18, wherein R¹ is —SH, —SC(O)CH₃, or —SSCH₃.

Embodiment P23. The compound of one of embodiments P1 to P18, wherein R¹ is E; and

E is

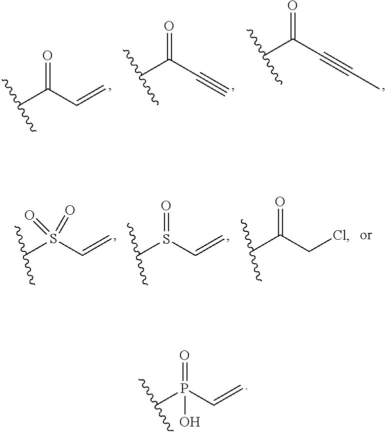

Embodiment P24. The compound of embodiment P1, wherein the compound has the formula

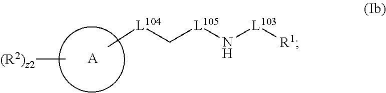

L¹⁰⁴ is a bond, —O—, —NH—, —S—, or substituted or unsubstituted alkylene;
=105 1—, is —S(O)₂—, —C(O)—, —NHC(O)—, or —OC(O)—; and
L¹⁰³ is a bond, substituted or unsubstituted alkylene, or substituted or unsubstituted heteroalkylene.

Embodiment P25. The compound of embodiment P24, wherein Ring A is a C₆-C₁₀ aryl or 5 to 10 membered heteroaryl.

Embodiment P26. The compound of embodiment P24, wherein Ring A is a phenyl.

Embodiment P27. The compound of embodiment P24, wherein the compound has the formula

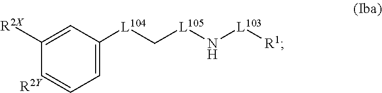

L¹⁰⁴ is a bond, —O—, —NH—, —S—, or substituted or unsubstituted C₁-C₄ alkylene;
L¹⁰⁵ is —S(O)₂—, —C(O)—, —NHC(O)—, or —OC(O)—;
L¹⁰³ is a bond, substituted or unsubstituted C₁-C₆ alkylene, or substituted or unsubstituted 2 to 6 membered heteroalkylene; and
R^{2X} and R^{2Y} are independently hydrogen, halogen, —CX²₃, —CHX²₂, —CH₂X², —OCX²₃, —OCH₂X², —OCHX²₂, —CN, —SO₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2X} and R^{2Y} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;.

Embodiment P28. The compound of embodiment P27, wherein R^{2X} and R^{2Y} are independently halogen.

Embodiment P29. The compound of embodiment P27, wherein R^{2X} and R^{2Y} are independently —Cl.

Embodiment P30. The compound of one of embodiments P24 to P29, wherein L¹⁰⁴ is —O—.

Embodiment P31. The compound of one of embodiments P24 to P30, wherein L¹⁰⁵ is —C(O)—.

Embodiment P32. The compound of one of embodiments P24 to P31, wherein L¹⁰³ is an unsubstituted alkylene.

Embodiment P33. The compound of one of embodiments P24 to P31, wherein L¹⁰³ is an unsubstituted C₁-C₆ alkylene.

Embodiment P34. The compound of one of embodiments P24 to P31, wherein L¹⁰³ is an unsubstituted C₁-C₄ alkylene.

Embodiment P35. The compound of one of embodiments P24 to P31, wherein L¹⁰³ is a bond.

Embodiment P36. The compound of one of embodiments P24 to P29, wherein

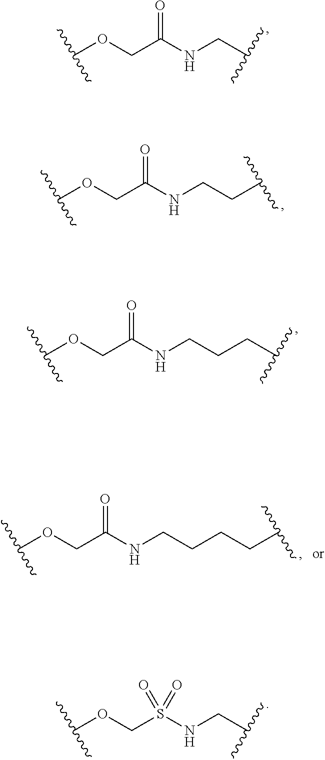 [SMILES: *CNC(=O)CO* ; *CCNC(=O)CO* ; *CCCNC(=O)CO* ; *CCCCNC(=O)CO* ; *CNS(=O)(=O)CO*]

-L¹⁰⁴-CH₂-L¹⁰⁵-NH-L¹⁰³—is

Embodiment P37. The compound of one of embodiments P24 to P36, wherein
R¹ is hydrogen, —SR^{1D}, —NR^{1A}R^{1B}, —OR^{1D}, —NR^{1A}SO₂R^{1D}, —NR^{1A}C(O)R^{1C}, E, substituted or unsubstituted C₁-C₆ alkyl, substituted or unsubstituted 2 to 6 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl;
E is an electrophilic moiety;
R^{1A}, R^{1B}, R^{1C}, and R^{1D} are independently hydrogen, halogen, —CCl₃, —CF₃, —CI₃, —CH₂Cl, —CH₂Br, —CHCl₂, —CHBr₂, —CHF₂, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, substituted or unsubstituted C₁-C₆ alkyl, substituted or unsubstituted 2 to 6 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl.

Embodiment P38. The compound of one of embodiments P24 to P36, wherein
R¹ is hydrogen, —SR^{1D}, —NR^{1A}R^{1B}, —OR^{1D}, —NR^{1A}SO₂R^{1D}, —NR^{1A}C(O)R^{1C}, E, R¹⁰-substituted or unsubstituted C₁-C₆ alkyl, R¹⁰-substituted or unsubstituted 2 to 6 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl;
E is an electrophilic moiety;
R^{1a}, R^{1B}, R^{1C}, and R^{1D} are independently hydrogen, halogen, —CCl₃, —CF₃, —CI_{3,}, —CH₂Cl, —CH₂Br, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCHCl₂, —OCHBr₂, —OCHF₂, —OCHI₂ R¹⁰-substituted or unsubstituted C₁-C₆ alkyl, R¹⁰-substituted or unsubstituted 2 to 6 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl;
R¹⁰ is oxo, halogen, —CCl₃, —CF₃, —CI₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R″-substituted or unsubstituted C₁-C₄ alkyl, R¹¹-substituted or unsubstituted 2 to 4 membered heteroalkyl, R¹¹-substituted or unsubstituted C₅-C₆ cycloalkyl, R¹¹-substituted or unsubstituted 5 to 6 membered heterocycloalkyl, R″-substituted or unsubstituted phenyl, or R¹¹-substituted or unsubstituted 5 to 6 membered heteroaryl;
R¹¹ is oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R¹²-substituted or unsubstituted C₁-C₄ alkyl, R^{u}-substituted or unsubstituted 2 to 4 membered heteroalkyl, R^{u}-substituted or unsubstituted C₅-C₆ cycloalkyl, R^{u}-substituted or unsubstituted 5 to 6 membered heterocycloalkyl, R^{u}-substituted or unsubstituted phenyl, or R^{u}-substituted or unsubstituted 5 to 6 membered heteroaryl; and
R¹² is oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment P39. The compound of one of embodiments P24 to P38, wherein R¹ is E; and

E is

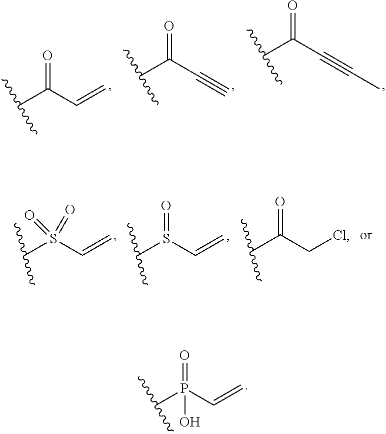

Embodiment P40. The compound of one of embodiments P1 to P39, wherein the compound is not

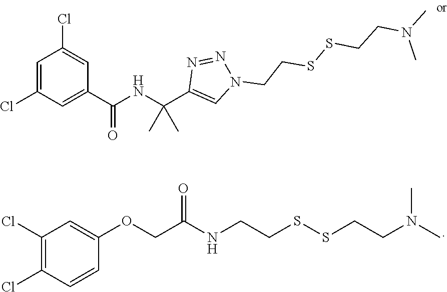 [SMILES: CN(C)CCSSCCn1cc(C(C)(C)NC(=O)c2cc(Cl)cc(Cl)c2)nn1 ; CN(C)CCSSCCNC(=O)COc1ccc(Cl)c(Cl)c1]

Embodiment P41. A pharmaceutical composition comprising a compound of one of embodiments P1 to P40 and a pharmaceutically acceptable excipient.

Embodiment P42. A method for treating a disease associated with dysregulation and/or degeneration of dopaminergic neurons in the central nervous system of a subject in need thereof, said method comprising administering to the subject in need thereof a therapeutically effective amount of a compound of one of embodiments P1 to P40.

Embodiment P43. The method of embodiment P42, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

Embodiment P44. The method of one of embodiments P42 to P43, wherein said disease associated with dysregulation and/or degeneration of dopaminergic neurons is Parkinson's disease.

Embodiment P45. A method of modulating the level of activity of Nurr1 in a subject in need thereof, the method comprising administering to the subject in need thereof an effective amount of a compound of one of embodiments P1 to P40.

Embodiment P46. A method of increasing the level of activity of Nurr1 in a cell, the method comprising contacting said cell with a compound of one of embodiments P1 to P40.

Embodiment P47. A method of increasing the level of dopamine in a cell, the method comprising contacting said cell with a compound of one of embodiments P1 to P40.

VI. Additional Embodiments

Embodiment 1. A compound having the formula

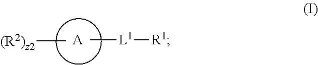

wherein
Ring A is aryl or heteroaryl;
L¹ is L¹⁰¹-L¹⁰²-L¹⁰³,
L¹⁰¹ is a bond, —S(O)₂—, —N(R¹⁰¹)—, —O—, —S—, —C(O)—, —C(O)N(R¹⁰¹)—, —(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted, , arylene, substituted or unsubstituted heteroarylene L¹⁰⁴-L¹⁰⁵, L¹⁰⁴-NH-L¹⁰⁵, or L¹⁰⁴-CH₂-L¹⁰⁵,
L¹⁰² is a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —O—, —NH—, —S—, —S(O)₂—, —C(O)—, —NHC(O)—, —C(O)NH—, —OC(O)—, —C(O)O—, substituted or unsubstituted alkylene, or substituted or unsubstituted heteroalkylene;
L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene;
R¹⁰¹, R¹⁰², and R¹⁰³ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SC1,₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SSR^{1D}, —SiR^{1A}R^{1B}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
R² is independently halogen, —CX²₃, —CHX²₂, —CH₂X², —OCX²₃, —OCH₂X², —OCHX²₂, —CN, —SOₙ₂R^{2D}, —SO_{b2}NR^{2A}R^{2B}, —NHC(O)NR^{2A}R^{2B}, —N(O).2, —NR^{2A}R^{2B}, —C(O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)_{R}^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1, and v2 are independently 1 or 2;
X¹ and X² are independently —F, —Cl, —Br, or —I; and
z2 is an integer from 0 to 5.

Embodiment 2. The compound of embodiment 1, wherein the compound has the formula

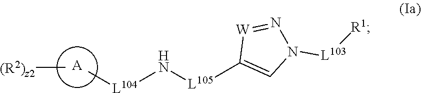

wherein
L¹⁰⁴ is a bond, —S(O)₂—, —C(O)—, —NHC(O)—, —OC(O)—, substituted or unsubstituted alkylene, or substituted or unsubstituted heteroalkylene;
L¹⁰⁵ is a bond, substituted or unsubstituted alkylene, substituted or unsubstituted heteroalkylene, substituted or unsubstituted cycloalkylene, or substituted or unsubstituted heterocycloalkyl ene;
L¹⁰³ is a bond, substituted or unsubstituted alkylene, or substituted or unsubstituted heteroalkylene; and
W is N or CH.

Embodiment 3. The compound of embodiment 2, wherein Ring A is a phenyl or 5 to 10 membered heteroaryl.

Embodiment 4. The compound of embodiment 2, wherein Ring A is a phenyl.

Embodiment 5. The compound of embodiment 2, wherein Ring A is a 3-quinolinyl.

Embodiment 6. The compound of one of embodiments 2 to 4, wherein the compound has the formula

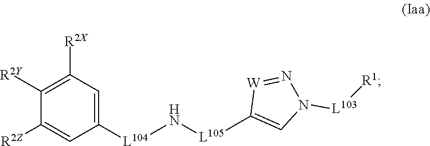

and
R^{2X}, -R^{2Y}, and R^{2Z} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}_{R}^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2X} and R^{2Y} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl; R^{2Y} and R^{2Z} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

Embodiment 7. The compound of embodiment 6, wherein
R^{2X} is independently halogen or unsubstituted heteroalkyl;
R^{2Y} is independently hydrogen or halogen; and
R^{2Z} is independently hydrogen, halogen, —CN, —NR^{2A}C(O)R^{2C}, unsubstitued heteroalkyl, or substituted or unsubstituted heterocycloalkyl.

Embodiment 8. The compound of embodiment 6, wherein
R^{2X} is independently halogen;
R^{2Y} is independently halogen; and
R^{2Z} is independently hydrogen.

Embodiment 9. The compound of embodiment 6, wherein
R^{2X} is independently —OCH₃;
R^{2Y} is independently hydrogen; and
R^{2Z} is independently —OCH₃.

Embodiment 10. The compound of embodiment 6, wherein
R²X is independently halogen or unsubstituted 2 to 4 membered heteroalkyl;
R^{2Y} is independently hydrogen;
R^{2Z} is independently halogen, —CN, —NR^{2A}C(O)R^{2C}, unsubstituted 2 to 4 membered heteroalkyl, or substituted or unsubstituted 5 to 6 membered heterocycloalkyl;
R^{2A} is independently hydrogen; and
R^{2C} is independently unsubstituted C₁-C₂ alkyl.

Embodiment 11. The compound of one of embodiments 2 to 10, wherein L¹⁰⁴ is —C(O)—.

Embodiment 12. The compound of one of embodiments 2 to 11, wherein L¹⁰⁵ is an unsubstituted alkylene.

Embodiment 13. The compound of one of embodiments 2 to 11, wherein L¹⁰⁵ is an unsubstituted C₁-C₄ alkylene.

Embodiment 14. The compound of one of embodiments 2 to 11, wherein L¹⁰⁵ is

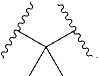 [SMILES: *C(*)(C)C]

Embodiment 15. The compound of one of embodiments 2 to 14, wherein W is N.

Embodiment 16. The compound of one of embodiments 2 to 15, wherein L¹⁰³ is an unsubstituted alkylene.

Embodiment 17. The compound of one of embodiments 2 to 15, wherein L¹⁰³ is an unsubstituted C₁-C₄ alkylene.

Embodiment 18. The compound of one of embodiments 2 to 15, wherein L¹⁰³ is an unsubstituted ethylene.

Embodiment 19. The compound of one of embodiments 2 to 10, wherein

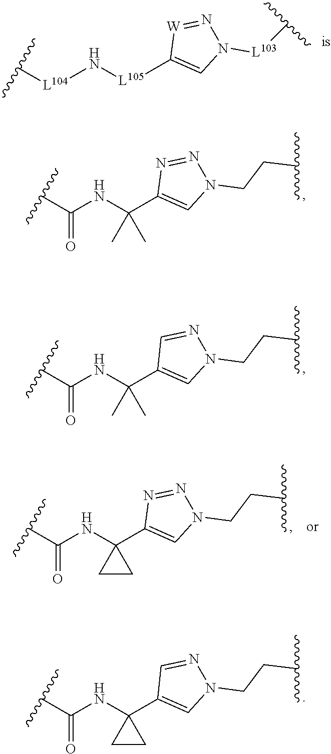

Embodiment 20. The compound of one of embodiments 1 to 19, wherein
_{R1 is —SR}^{1D}, —NR^{1A}R^{1B}, —OR^{1D}, , unsubstituted alkyl, substituted or unsubstituted phenyl, or substituted or unsubstituted 5 to 6 membered heteroaryl;
R^{1A} is independently hydrogen or unsubstituted C₁-C₄ alkyl;
R^{1B} is independently hydrogen or unsubstituted C₁-C₄ alkyl; and
R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, or substituted or unsubstituted alkyl.

Embodiment 21. The compound of one of embodiments 1 to 19, wherein
R¹ is —SR^{1D}, —NR^{1A}R^{1B}, —⁰R^{1D}, E, unsubstituted C₁-C₄ alkyl, R¹⁰-substituted or unsubstituted phenyl, or R¹⁰-substituted or unsubstituted 5 to 6 membered heteroaryl;
R^{1A} is independently hydrogen or unsubstituted C₁-C₄ alkyl;
R^{1B} is independently hydrogen or unsubstituted C₁-C₄ alkyl;
R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl; and
R¹⁰ is oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment 22. The compound of one of embodiments 1 to 19, wherein R¹ is —SR^{1D} or R¹⁰-substituted phenyl;

R^{1D} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, —PO₃H₂, R¹⁰-substituted or unsubstituted C₁-C₄ alkyl; and

R¹⁰ is oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment 23. The compound of one of embodiments 1 to 19, wherein R¹ is —SH, —SC(O)CH₃, or —SSCH₃.

Embodiment 24. The compound of one of embodiments 1 to 19, wherein R¹ is E; and

E is

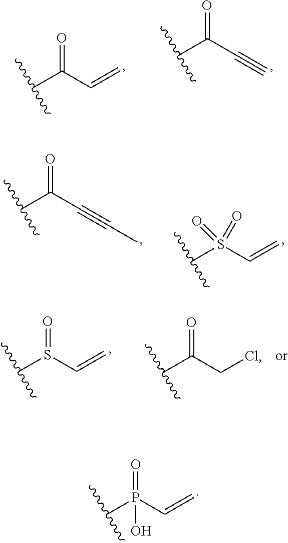

Embodiment 25. The compound of embodiment 1, wherein the compound has the formula

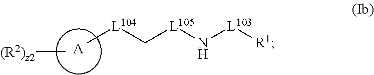

L¹⁰⁴ is a bond, —O—, —NH—, —S—, or substituted or unsubstituted alkylene;
L¹⁰⁵ is —S(O)₂—, —C(O)—, —NHC(O)—, or —OC(O)—; and
L¹⁰³ is a bond, substituted or unsubstituted alkylene, or substituted or unsubstituted heteroalkylene.

Embodiment 26. The compound of embodiment 25, wherein Ring A is a C₆-C₁₀ aryl or 5 to 10 membered heteroaryl.

Embodiment 27. The compound of embodiment 25, wherein Ring A is a phenyl.

Embodiment 28. The compound of embodiment 25, wherein the compound has the formula

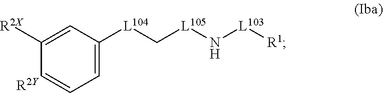

L¹⁰⁴ is a bond, —O—, —NH—, —S—, or substituted or unsubstituted C₁-C₄ alkylene;
L¹⁰⁵ is —S(O)₂—, —C(O)—, —NHC(O)—, or —OC(O)—;
L¹⁰³ is a bond, substituted or unsubstituted C₁-C₆ alkylene, or substituted or unsubstituted 2 to 6 membered heteroalkylene; and
R^{2X} and R^{2Y} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}_{R}^{2B}, —NHC(O)NR^{2A}_{R}^{2B}, —N(O)ₘ₂, —NR^{2A}_{R}^{2B}, —C(O)R^{2C}, —C(O)OR^{2C}, —C(O)NR^{2A}_{R}^{2B}, —OR^{D2}, —NR^{2A}SO2R^{D2}, —NR^{2A}C(O)_{R}^{2C}, —NR^{2A}C(O)OR^{2C}, —NR^{2A}OR^{2C},—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2X} and R^{2Y} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

Embodiment 29. The compound of embodiment 28, wherein R^{2X} and R^{2Y} are independently halogen.

Embodiment 30. The compound of embodiment 28, wherein R^{2X} and R^{2Y} are independently —Cl.

Embodiment 31. The compound of one of embodiments 25 to 30, wherein L¹⁰⁴ is —O—.

Embodiment 32. The compound of one of embodiments 25 to 31, wherein L¹⁰⁵ is —C(O)—.

Embodiment 33. The compound of one of embodiments 25 to 32, wherein L¹⁰³ is an unsubstituted alkylene.

Embodiment 34. The compound of one of embodiments 25 to 32, wherein L¹⁰³ is an unsubstituted C₁-C₆ alkylene.

Embodiment 35. The compound of one of embodiments 25 to 32, wherein L¹⁰³ is an unsubstituted C₁-C₄ alkylene.

Embodiment 36. The compound of one of embodiments 25 to 32, wherein L¹⁰³ is a bond.

Embodiment 37. The compound of one of embodiments 25 to 30, wherein -L¹⁰⁴-CH₂-L¹⁰⁵-NH-L^{103—is}

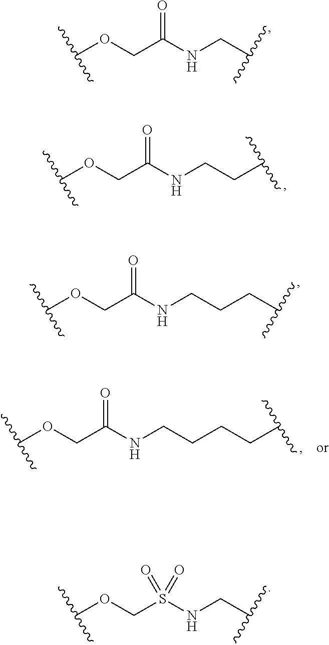 [SMILES: *CNC(=O)CO* ; *CCNC(=O)CO* ; *CCCNC(=O)CO* ; *CCCCNC(=O)CO* ; *CNS(=O)(=O)CO*]

Embodiment 38. The compound of one of embodiments 25 to 37, wherein
R¹ is hydrogen, —SR^{1D}, —NR^{1A}R^{1B}, —OR^{1D}, —NR^{1A}SO₂R^{1D}, —NR^{1A}C(O)R^{1C}, E, substituted or unsubstituted C₁-C₆ alkyl, substituted or unsubstituted 2 to 6 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl;
E is an electrophilic moiety;
R^{1A}, R^{1B}, R^{1C} and R^{1D} are independently hydrogen, halogen, —CCl₃, —CBr₃, —CF₃, —CI_{3,}, —CH₂Cl, —CH₂Br, —CH₂F, —CH₂I, —CHCl₂, —CHBr₂, —CHF₂, —CHI₂—CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted C₁-C₆ alkyl, substituted or unsubstituted 2 to 6 membered heteroalkyl, substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl.

Embodiment 39. The compound of one of embodiments 25 to 37, wherein
R¹ is hydrogen, —SR^{1D}, —NR^{1A}R^{1B}, —OR^{1D}, —NR^{1A}SO₂R^{1D}, —NR^{1A}C(O)R^{1C}, E, R¹⁰-substituted or unsubstituted C₁-C₆ alkyl, R¹⁰-substituted or unsubstituted 2 to 6 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl;
E is an electrophilic moiety;
R^{1C} and R^{ip} are independently hydrogen, halogen, —CCl₃, —CBr₃, —CF₃, —CI_{3,}, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, R¹⁰-substituted or unsubstituted C₁-C₆ alkyl, R¹⁰-substituted or unsubstituted 2 to 6 membered heteroalkyl, R¹⁰-substituted or unsubstituted C₅-C₆ cycloalkyl, R¹⁰-substituted or unsubstituted 5 to 6 membered heterocycloalkyl, R¹⁰-substituted or unsubstituted phenyl, or R¹⁰-substituted or unsubstituted 5 to 6 membered heteroaryl;
R¹⁰ is oxo, halogen, —CCl₃, —CBr₃, —CF₃, —CI₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R¹¹-substituted or unsubstituted C₁-C₄ alkyl, R¹¹-substituted or unsubstituted 2 to 4 membered heteroalkyl, R¹¹-substituted or unsubstituted C₅-C₆ cycloalkyl, R″-substituted or unsubstituted 5 to 6 membered heterocycloalkyl, R¹¹-substituted or unsubstituted phenyl, or R¹¹-substituted or unsubstituted 5 to 6 membered heteroaryl;
R¹¹ is oxo, halogen, —CCl₃, —CBr₃, —CF₃, —CI₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R¹²-substituted or unsubstituted C₁-C₄ alkyl, R^{u}-substituted or unsubstituted 2 to 4 membered heteroalkyl, R¹²-substituted or unsubstituted C₅-C₆ cycloalkyl, R¹²-substituted or unsubstituted 5 to 6 membered heterocycloalkyl, R¹²-substituted or unsubstituted phenyl, or R^{u}-substituted or unsubstituted 5 to 6 membered heteroaryl; and
R¹² is oxo, halogen, —CCl₃, —CBr₃, —CF₃, —CI₃, —CH₂Cl, —CH₂Br, —CH₂F, —CHCl₂, —CHBr₂, —CHF₂, —OCCl₃, —OCBr₃, —OCF₃, —OCI₃, —OCH₂Cl, —OCH₂Br, —OCH₂F, —OCHCl₂, —OCHBr₂, —OCHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted C₁-C₄ alkyl, unsubstituted 2 to 4 membered heteroalkyl, unsubstituted C₅-C₆ cycloalkyl, unsubstituted 5 to 6 membered heterocycloalkyl, unsubstituted phenyl, or unsubstituted 5 to 6 membered heteroaryl.

Embodiment 40. The compound of one of embodiments 25 to 39, wherein R¹ is E; and

E is

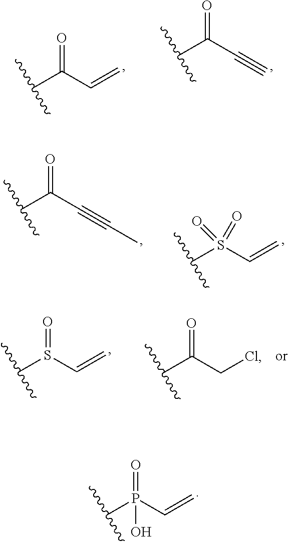

Embodiment 41. The compound of one of embodiments 1 to 40, wherein the

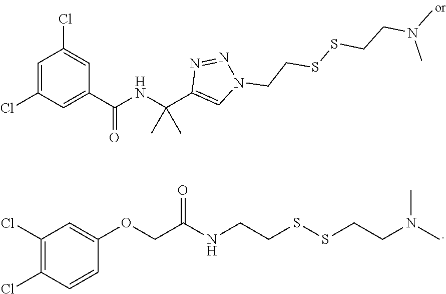 [SMILES: CN(C)CCSSCCn1cc(C(C)(C)NC(=O)c2cc(Cl)cc(Cl)c2)nn1 ; CN(C)CCSSCCNC(=O)COc1ccc(Cl)c(Cl)c1]

compound is not

Embodiment 42. A pharmaceutical composition comprising a compound of one of embodiments 1 to 41 and a pharmaceutically acceptable excipient.

Embodiment 43. A method for treating a disease associated with dysregulation and/or degeneration of dopaminergic neurons in the central nervous system of a subject in need thereof, said method comprising administering to the subject in need thereof a therapeutically effective amount of a compound of one of embodiments 1 to 41.

Embodiment 44. The method of embodiment 43, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

Embodiment 45. The method of one of embodiments 43 to 44, wherein said disease associated with dysregulation and/or degeneration of dopaminergic neurons is Parkinson's disease.

Embodiment 46. A method of treating a cancer in a subject in need thereof, the method comprising administering to the subject in need thereof a therapeutically effective amount of a compound of one of embodiments 1 to 41.

Embodiment 47. The method of embodiment 46, wherein said cancer is breast cancer, pancreatic cancer, bladder cancer, mucoepidermoid carcinoma, gastric cancer, prostate cancer, colorectal cancer, lung cancer, adrenocortical cancer, or cervical cancer.

Embodiment 48. A method of modulating the level of activity of Nurr1 in a subject in need thereof, the method comprising administering to the subject in need thereof an effective amount of a compound of one of embodiments 1 to 41.

Embodiment 49. A method of increasing the level of activity of Nurr1 in a cell, the method comprising contacting said cell with a compound of one of embodiments 1 to 41.

Embodiment 50. A method of increasing the level of dopamine in a cell, the method comprising contacting said cell with a compound of one of embodiments 1 to 41.

Embodiment 51. A pharmaceutical composition comprising 5,6-dihydroxyindole (DHI) and a pharmaceutically acceptable excipient.

Embodiment 52. A method for treating a disease associated with dysregulation and/or degeneration of dopaminergic neurons in the central nervous system of a subject in need thereof, said method comprising administering to the subject in need thereof a therapeutically effective amount of 5,6-dihydroxyindole (DHI).

Embodiment 53. The method of embodiment 52, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

Embodiment 54. A method of treating a cancer in a subject in need thereof, the method comprising administering to the subject in need thereof a therapeutically effective amount of 5,6-dihydroxyindole (DHI).

Embodiment 55. The method of embodiment 54, wherein said cancer is breast cancer, pancreatic cancer, bladder cancer, mucoepidermoid carcinoma, gastric cancer, prostate cancer, colorectal cancer, lung cancer, adrenocortical cancer, or cervical cancer.

### EXAMPLES

### Example 1: Nurr1 (NR4A2) Receptor Modulators

Over one million Americans are currently living with Parkinson's disease (PD), and approximately 60,000 new cases are diagnosed each year (Wirdefeldt et al., 2011). In an estimated 90% of PD patients, the cause of the disease is unknown, having no clear genetic or environmental origin (de Lau and Breteler, 2006). The most pronounced neuropathological feature of PD is the progressive degeneration of dopaminergic neurons in the substantia nigra pars compacta and the consequent reduction in dopamine levels in the striatum, which manifest as impairments in motor function (e.g., rigidity, tremor, bradykinesia) (Samii et al., 2004). Notably, this degeneration appears to be preceded by the loss of the dopaminergic phenotype; that is, at least some dopaminergic neurons first stop producing and signaling with dopamine prior to degenerating (Janezic et al., 2013). Although the molecular basis for idiopathic PD remains incompletely understood, it has been proposed to include oxidative stress, mitochondrial dysfunction, and dysregulation of dopamine homeostasis (Blesa et al., 2015; Hauser and Hastings, 2013; Hwang, 2013). Currently, there are no available treatments that stop or even slow the progression of PD. Existing therapeutics relieve PD symptoms by increasing dopaminergic signaling through one of three mechanisms: (1) increasing dopamine levels by augmenting the amount of its biosynthetic precursor, L-DOPA; (2) blocking the breakdown of dopamine by inhibiting its metabolic enzymes (monoamine oxidase (MAO), COMT); (3) mimicking the activity of dopamine by directly agonizing dopamine receptors. However, these drugs only partially alleviate symptoms and can have significant side effects, especially as the disease progresses. New types of therapeutics are desperately needed to combat both the symptoms and progression of PD.

Modulators of Nurr1 receptor activity have potential applications for the treatment of diseases associated with the dysregulation and/or degeneration of dopaminergic neurons in the central nervous system. These diseases include Parkinson's disease, multiple sclerosis, amyotrophic lateral sclerosis, schizophrenia, and drug addiction. Our efforts are currently focused on developing Nurr1 modulators to treat the symptoms and progression of PD (Campos-Melo et al., 2013; Decressac et al., 2013; Dong et al., 2016; Johnson et al., 2011; Kim et al., 2015). Mounting evidence also suggests Nurr1 is a therapeutic target for Alzheimer's disease (Moon et al., 2018).

Small molecule modulators of Nurr1 function may be used to (1) stimulate the development of dopaminergic neurons from stem cells, (2) support the health of mature dopaminergic neurons, (3) prevent the degeneration of mature dopaminergic neurons, (4) stimulate the synthesis of dopamine in neurons. Diseases that would be impacted by these functions include Parkinson's disease, multiple sclerosis, amyotrophic lateral sclerosis, schizophrenia, and drug addiction. For most indications, a Nurr1 agonist is likely the desired activity. However, the biology of Nurr1 is incompletely understood and for some indications an antagonist may prove valuable.

A handful of putative Nurr1 agonists have been reported in the patent and scientific literature (Dong et al., 2016). With the exception of amodiaquine (Kim et al., 2015), there is little evidence that any of these compounds bind directly to Nurr1. Our invention identifies ligands that both bind directly to the Nurr1 and modulate Nurr 1 activity in cells.

Herein, we disclose small molecules that bind directly to and modulate the activity of the transcription factor nuclear receptor related-1 protein (Nurr1), also known as NR⁴A2. Nurr1 regulates the expression of genes critical for the development, maintenance, and survival of dopaminergic neurons (Alavian et al., 2014; Jankovic et al., 2005; Johnson et al., 2011; Kadkhodaei et al., 2009; Luo, 2012; Zetterstrom et al., 1997). In particular, Nurr1 plays a fundamental role in maintaining dopamine homeostasis by regulating transcription of the genes governing dopamine synthesis (TH, tyrosine hydroxylase; DDC, dopa decarboxylase), packaging (SLC₁₈A2, vesicular monoamine transporter 2, VMAT2), and reuptake (DAT, dopamine transporter, also known as SLC₆A3) (Hermanson et al., 2003; Iwawaki et al., 2000; Johnson et al., 2011; Sacchetti et al., 2001). Nurr1 also regulates the survival of dopaminergic neurons by stimulating the transcription of genes coding for neurotrophic factors (BDNF, NGF), anti-inflammatory responses (GDNF receptor c-Ret), and oxidative stress management (SOD1), as well as repressing the transcription of pro-inflammatory genes (TNFalpha, iNOS, IL-lbeta) (Galleguillos et al., 2010; Johnson et al., 2011; Kadkhodaei et al., 2013; Kim et al., 2003; Saijo et al., 2009; Sakurada et al., 1999; Volpicelli et al., 2007). Nurr1 is a potential therapeutic target for several diseases associated with the dysregulation and/or degeneration of dopaminergic neurons (e.g., multiple sclerosis, amyotrophic lateral sclerosis, schizophrenia, drug addiction), especially Parkinson's disease (Campos-Melo et al., 2013; Decressac et al., 2013; Dong et al., 2016; Johnson et al., 2011; Kim et al., 2015). Some evidence also suggests Nurr1 is a therapeutic target for Alzheimer's disease (Moon et al., 2018).

Validation of Nurr1 as a PD therapeutic is primarily derived from mouse models and human data. Homozygous mice lacking Nurr1 fail to generate midbrain dopaminergic neurons and die shortly after birth, heterozygous mice have motor impairments analogous to Parkinsonian deficits, and conditional ablation of Nurr1 in adult animals recapitulates early features of PD with progressive dopaminergic neuropathology (Jiang et al., 2005; Kadkhodaei et al., 2013; Kadkhodaei et al., 2009; Zetterstrom et al., 1997; Zhang et al., 2012). In patients with PD, the expression of Nurr1 is reduced compared to age-matched controls (Chu et al., 2006; Le et al., 2008; Montarolo et al., 2016; Moran et al., 2007), though only a few, rare polymorphisms in Nurr1 appear to be associated with the disease (Grimes et al., 2006; Le et al., 2003). Stimulation of Nurr1 activity may combat both the reduced dopamine levels and the increased oxidative stress associated with PD.

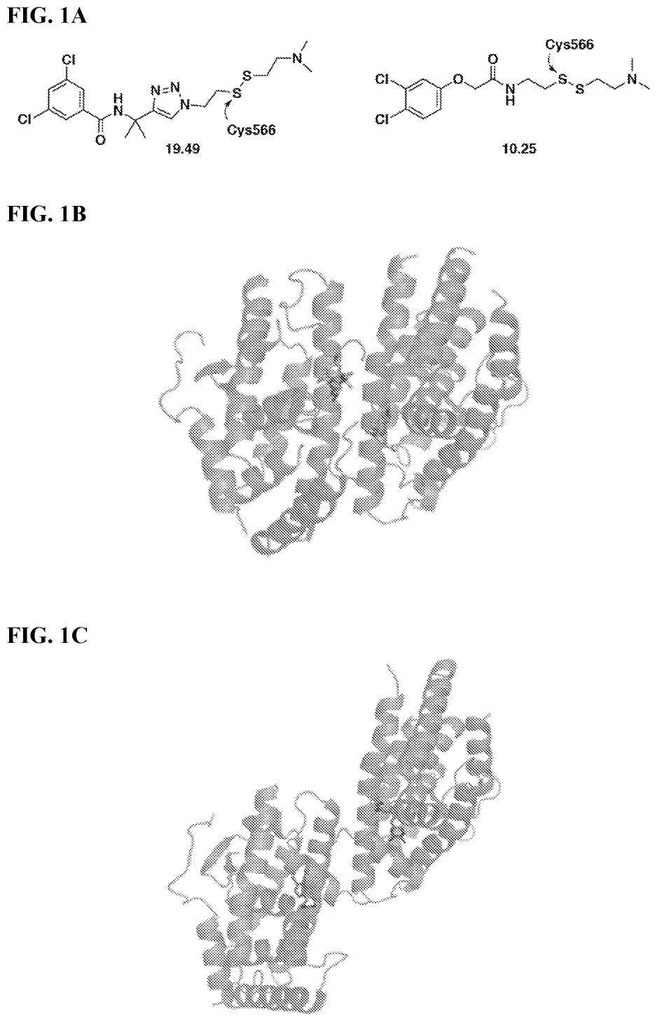

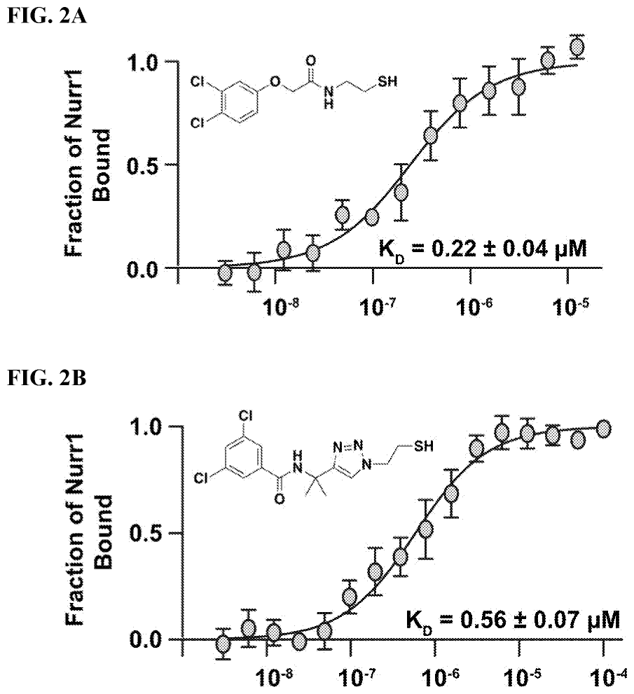

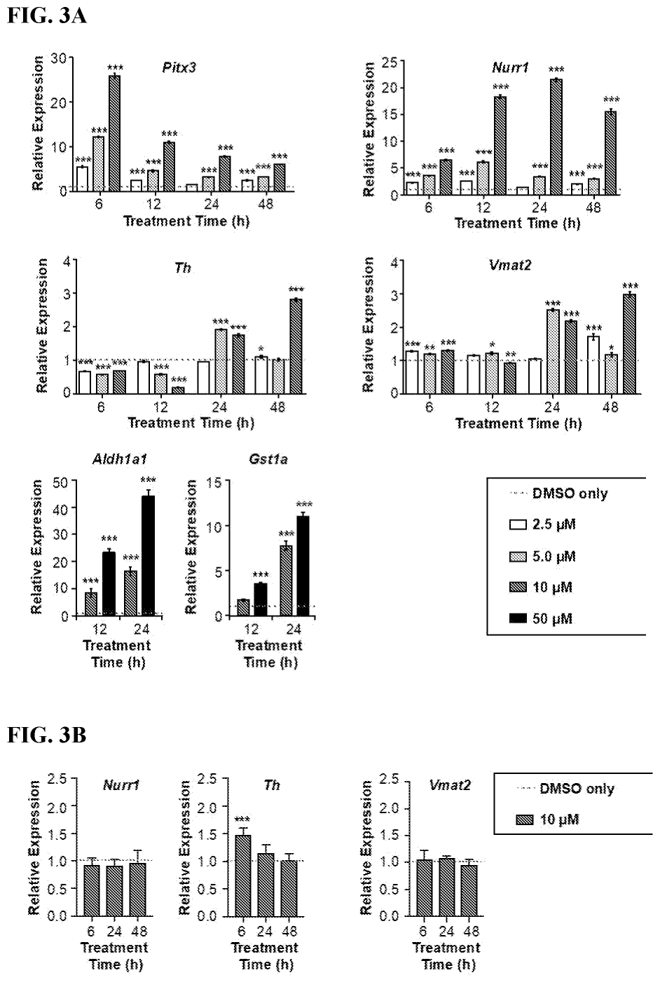

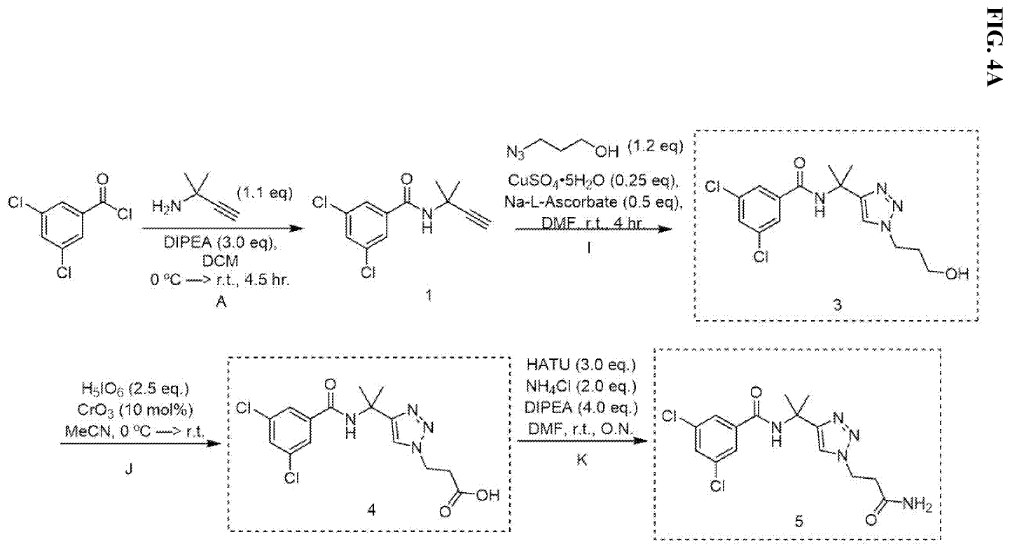

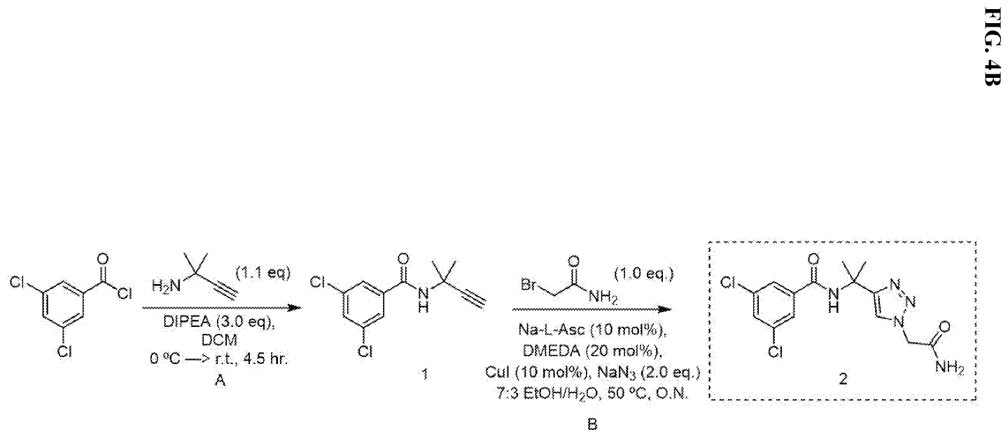

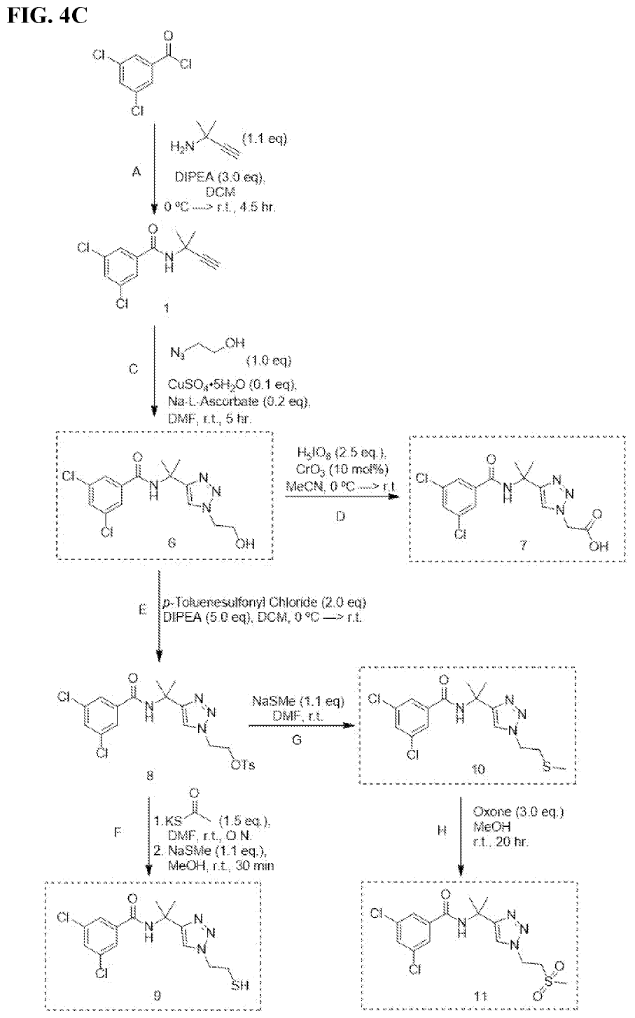

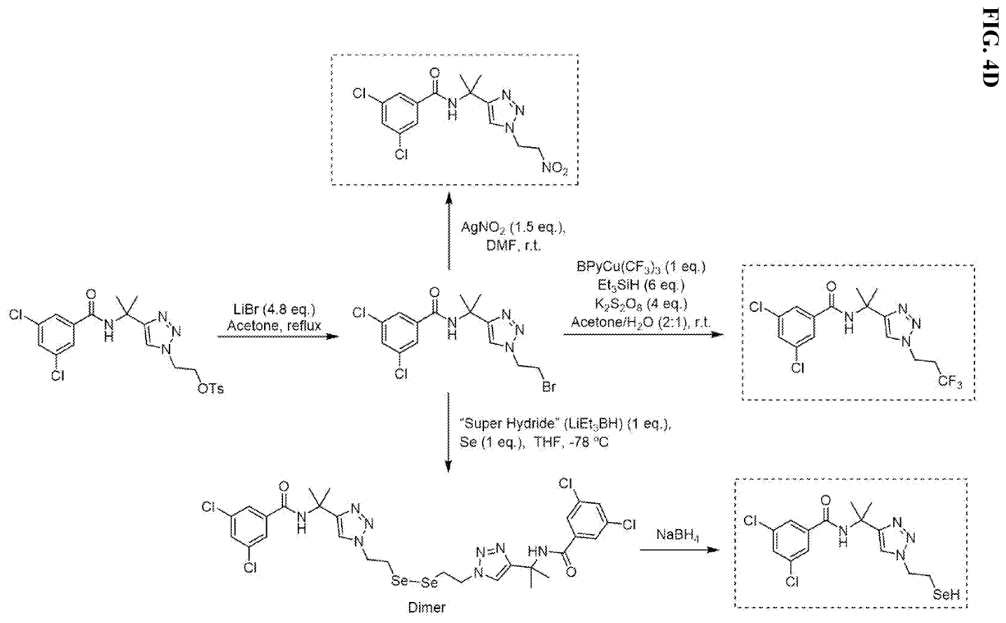

The small molecule modulators of Nurr1 activity described herein were developed as follows. First, we used the Nurr1 ligand binding domain (LBD) and a disulfide-trapping screen to identify 50 compounds that conjugate directly to Nurr1, undergoing a disulfide exchange reaction with Cys566. Next, we solved crystal structures of two of the top screening hits (10.25 and 19.49) covalently bound to Nurr1 (FIG. 1A, FIG. 1B), defining two distinct ligand binding pockets within the Nurr1 ligand binding domain and thus providing a rational basis for improving ligand affinity and efficacy. We also solved the structure of Nurr1 bound to a dopamine metabolite (Bruning et al., 2019); the metabolite binds at a site between where the two screening hits bind. Only one other crystal structure of Nurr1 has been published, and it is without any bound ligand (Wang et al., 2003).

Based on these data, analogs of two screening hits (10.25, 19.49) were synthesized and characterized in terms of their affinity and efficacy in vitro. In particular, direct binding to the Nurr1 LBD in vitro was measured using microscale thermophoresis (MST) and surface plasmon resonance (SPR), and efficacy in cells was measured using a lucifierase reporter assay (Nurr1 LBD fused to Gal4 DBD, measuring effect on luciferase activity) and target gene transcription assays (full length Nurr1, measuring mRNA levels of specific Nurr1 target genes). These assays pointed to a subset of compounds that bind directly to the Nurr1 LBD (by MST and/or SPR) and produce >1.5-fold changes in Nurr1 activity in cells (by Luc and/or TGT assays) (Table 1, Table 2).

Specifically, we identified 15 compounds derived from the screening hit 19.49 that bind to the Nurr1 LBD with micromolar affinity in direct binding assays (MST, SPR), and modulate the activity of Nurr1 in cellular assays (luciferase reporter assay, target gene transcription assays examining Nurr1, Pitx3, TH, VMAT2 transcripts). Of these 15 compounds, five are clearly agonists. We also identified 11 compounds derived from the screening hit 10.25 that bind to the Nurr1 LBD with micromolar affinity in direct binding assays (MST, SPR) and activate Nurr1 in the luciferase reporter assay. Analogs of 10.25 are being tested in target gene transcription assays.

Ongoing steps to validate the invention (with respect to Parkinson's disease) include: (1) quantification of ligand effects on the transcription of Nurr1 target genes in other cells (e.g., SH—SY5Y, MND9 cells, acute dissociated dopaminergic neurons); (2) quantification of ligand effects on the survival isolated cells in PD models (e.g., rotenone- and 6-hydroxydopamine-treated cells); (3) quantifying ligand effects, including blood brain barrier permeability, in Parkinson's disease mouse models; and (4) developing additional analogs with improved PK/PD properties and affinity, as needed.

### Example 2: Development of Compounds that Stabilize Specific Conformations of Nurr1

Efforts to drug Nurr1 have been largely unsuccessful, hampered by major gaps in the understanding of the receptor's structure and regulation. In particular, the only reported crystal structure of the receptor (apo Nurr1), published over 14 years ago, shows the canonical NR ligand binding pocket is occupied by bulky amino acid side chains (33). Endogeneous ligands for Nurr1 have yet to be reported, further limiting our understanding of how this receptor is regulated. A small number of synthetic ligands for Nurr1 have been described in the scientific and patent literature, and reported to up-regulate transcription and protein levels of Nurr1 target genes in vivo; provide some degree of neuroprotection; and improve behavioral deficits in mouse models (7,20,38,39-42). However, there is little evidence that any of these “Nurr1 agonists” directly activate the receptor, with the possible exception of recent work on the antimalarial amodiaquine (20). Efforts to drug Nurr1 indirectly, targeting RXR in Nurr1:RXR heterodimers, have produced some intriguing, yet contradictory effects, and the precise mechanism of action for enhanced expression of Nurr1 target genes by RXR agonists is unclear (41,43,44). For example, Perlmann showed that the transcriptional activity of Nurr1 itself is reduced upon complex formation with RXR (45). In any case, strategies targeting Nurr1:RXR heterodimers do not preclude approaches aimed at directly activating Nurr1. Moreover, it is of great interest to address whether these two strategies will exhibit synergistic effects. Against this backdrop, we utilized an orthogonal screening technology called disulfide-trapping (or tethering) combined with biophysical and structural assays to identify Nurr1 ligands having well-defined binding sites.

There are five cysteine residues in Nurr1, but only three of them formed adducts in the disulfide-trapping screen. In particular, ˜50 compounds reacted with Cys566, five reacted with Cys475 (adjacent to Cys566 in the LBD), and 10 reacted with Cys534 (on the surface of the LBD). Based on these data, we expect the primary site of modification will be Cys566 within the LBD.

Crystallographic evidence that disulfide-linked ligands stabilize distinct conformations of Nurr1. We solved crystal structures for two of the screening hits (10.25 and 19.49) covalently bound to Nurr1. While both ligands stabilize head-to-tail Nurr1 homodimers, the overall structures are significantly different. The 10.25 homodimer is similar to the homodimer seen for Nur77 (a structurally related member of the NR⁴A subfamily of nuclear receptors (NRs)), but the 19.49 homodimer represents a new conformation not previously seen among NRs (46,47). The PISA scores (48,49) for each dimer (1.0 for 10.25, 0.93 for 19.49) indicate that both dimers represent biologically relevant assemblies of the protein, rather than a reflection of crystal packing forces. Notably, both structures preclude the formation of Nurr1:RXR heterodimers (assuming the interaction surface is similar to that observed for other RXR heterodimers). The spatial arrangement of the NR DNA binding domains (DBDs) attached to the ligand binding domains (LBDs) (among other DNA-related factors) determines which DNA sequences are recognized by an NR complex. In particular, NRs discriminate between binding sites (DNA response elements) by recognizing the orientations and spacing of two DNA half-sites, to direct sequence-specific gene activity (50). Full-length structures of two different RXR:NR heterodimers complexed with DNA underscore this relationship (51,52). Extrapolating from our structures the relative distances between the DBDs, by measuring the distances between N-termini, these two Nurr1 homodimers recognize distinct DNA response elements.

Identification of an endogenous Nurr1 ligand. Dopamine is broken down inside the neurons that produce it, generating oxygen free-radicals and other potentially damaging molecules (53). Among the reactive metabolites is 5,6-dihydroxyindole (DHI), a compound that undergoes spontaneous oxidation to a reactive quinone, which oligomerizes to form a polymer (neuromelanin) of unknown function. This polymer accounts for the dark appearance of nigrostriatal neurons in the normal adult CNS (54,55). Dopamine is also a central player in stress and addiction (3). It is clear that dopamine levels need to be tightly regulated in the CNS. Nurr1 controls all the genes required for dopamine synthesis, but its regulation is poorly understood as the receptor lacks both the canonical NR ligand binding pocket and the classic NR co-regulator binding surface (33). We postulated that Nurr1 might be regulated by dopamine itself, or one of its metabolites, and investigated this possibility using a combination of biophysical and structural techniques. These data revealed that DHI binds to Nurr1, forming a reversible covalent adduct with Cys566. Specifically, using differential scanning fluorimetry (DSF), we found that DHI (but not dopamine or other metabolites) stabilizes the Nurr1 LBD, increasing the melting temperature by one degree. Using surface plasmon resonance, we observed that DHI binds to Nurr1 with a K_{d} of 5 μM, and a very slow off-rate. Moreover, we solved an x-ray structure of DHI covalently bound to Nurr1. Just as in the apo structure, the protein crystallizes as a monomer. A shift of ˜1 Å in the position of Helix 12 relative to the apo structure suggests a physiological role for the interaction; Helix 12 is classically a key regulator of NR function. The structure shows that DHI, likely reacting as the indolequinione (DHIQ), forms a covalent adduct with Cys566. Finally, we showed DHI is active in cellular assays, stimulating Nurr1 activity in a classic reporter assay and driving transcription of Nurr1 target genes in live zebrafish. DHI drives the expression of VMAT (package dopamine in vesicles) following acute exposure (6 h, data not shown), and TH (make more dopamine) following longer exposure (24 h). Identifying stable analogs of DHI will enable more detailed studies of this intriguing biology.

Nurr1 binds to DNA as a monomer, homodimer, or heterodimer with retinoid X receptor (RXRa) (11,56-61). Based on extensive precedent in the NR field (37,50), we hypothesize that monomeric, homodimeric, and heterodimeric Nurr1 complexes will modulate discrete subsets of Nurr1 target genes. Nuclear receptors bind to specific DNA sequences (response elements) dictated by the spatial relationship between their attached DNA binding domains (DBDs), or to half-sequences (“half-sites”) in the case of monomers. The absence of pharmacological probes for Nurr1 has precluded clarifying the specific biological functions and target genes regulated by each of its known conformations. Building on our preliminary data, the following aims seek to develop ligands that bind directly to Nurr1 to specifically regulate the transcription of target genes underlying the development and maintenance of dopaminergic neurons.

Determining ligand effects on Nurr1 target gene transcription. We are focused on developing covalent Nurr1 ligands that can be used to enforce specific conformational states of Nurr1 inside of cells and, then, using those probes to identify the gene targets associated with each conformational state. In particular, we have synthesized analogs of our screening hits in which the disulfide electrophile is replaced with electrophiles suitable for intracellular studies, and then quantified the effects of those probes in cellular assays. Notably, some Nurr1 target genes have clear tandem NuRE binding elements, suggesting Nurr1 homodimers will increase transcription—but which one? We identified ligands that enforce two distinctly different Nurr1 homodimers conformations.

### Example 3: Experimental Details

Microscale thermophoresis (MST) assays. Data were collected using the Nanotemper Monolith NT.115 at a temperature of 25° C. The MST buffer used in each experiment was 25 mM HEPES (Sigma Aldrich), pH 7.4, 150 mM NaCl (Alfa Aesar), and 0.02% Pluronic (Sigma Aldrich). All samples were prepared using Protein LoBind tubes or deepwell plates (Eppendorf). The His-tagged Nurr1 was labeled with RED-tris-NTA dye (NT-647) according to the kit's protocol (Nanotemper). Dilutions of each ligand were carried out starting from DMSO stocks of 10 mM in DMSO. The analyte solutions in DMSO were added to aliquots of MST buffer to yield a DMSO concentration of 4%.

Two types of experiments were performed: endpoint and binding affinity assays. For the endpoint assays, equal amounts of the ligand solution and labeled Nurr1 were mixed to yield the final concentrations: 50 nM Nurr1, 25 nM RED-tris-NTA dye, 2% DMSO, and either 25 μM or 100 μM of the desired ligand in the MST buffer. A negative control was prepared with the final concentrations, 50 nM Nurr1, 25 nM RED-tris-NTA dye, and 2% DMSO in the MST buffer. After incubating for 5 minutes, the samples were loaded into Monolith NT.115 Premium Capillaries (Nanotemper).

For the binding affinity assays, a titration series of 1:1 dilutions were prepared starting from an aliquot of 200 μM ligand, 4% DMSO in the MST buffer. These dilutions were carried out with 4% DMSO in the MST buffer for a total of 16 dilutions. Equal amounts of labeled Nurr1 were added to each dilution in the titration series. After incubating for 20 minutes, the samples were loaded into Monolith NT.115 Premium Capillaries (Nanotemper).

The Monolith NT.115 settings for all samples were 40% excitation power and 40% MST power. The initial fluorescence was recorded for 3 seconds and the thermophoresis fluorescence response was recorded for 20 seconds. The MO. Screening Analysis software (Nanotemper) was used to normalize the fluorescent response signals to the sample's initial fluorescence. With this data, a plot of the fraction of Nurr1 bound to the ligand versus concentration of the ligand. This plot was then used to determine the dissociation constant (K_{d}) using the equation below.

Surface Plasmon Resonance Assays. Data were collected using Biacore T200 (GE) instrument at a flow rate of 30 μL/min and at a temperature of 25° C. The running buffer was 25 mM HEPES, pH 7.4, 150 mM NaCl, 0.05% Surfactant Tween 20, and 2% DMSO. The biotinylated Nurr1 LBD was immobilized on a sensor chip SA (GE Healthcare Life Sciences; product number 29104992) or at 6000-7000 RU, or a CAP chip (GE Healthcare Life Sciences; product number 28920234) at 1500-2000 RU. Data collection was performed with kinetic titration mode. Analytes dilutions were carried out starting from DMSO stocks (10 mM). Analytes dissolved in DMSO were added to 1.02x running buffer without DMSO to yield a final DMSO concentration of 2%. When using CAP chip, surface regeneration was performed between each titration curve using 6 M guanidine.HCl+0.25 M NaOH regeneration solution followed by re-immobilization of Nurr1 (as described in the manufacturer's regeneration protocol). Data processing included double referencing (i.e., reference flow cell and buffer subtracted using a buffer injection of appropriate contact time for the given injection). Solvent correction was performed using a standard curve in a range of 1.8-2.3% DMSO.

Luciferase Reporter Assays. pBIND—Nurr1 is generated by cloning Nurr1 LBD (a.a. 328-598 of human Nurr1) into pBIND vector (Promega E2440). The pBIND vector also contains a Renilla luciferase gene under the control of the SV40 Promoter that can be used to normalize the transfection efficiency. The firefly luciferase reporter pG5-Luc vector (Promega E2440) contains 5 repeats of GAL4 UAS (upstream activation sequence) upstream of luciferase gene. SK—N— BE(2)C cells (ATCC CRL-2268) were transiently transfected with pBIND—Nurr1 and pG5-Luc by FuGENE HD (Promega, E2311) in 96-well-plate with the seeding density of 200,000 cells/mL. 24 hours after transfection, cells were incubated with Nurr1 agonist at indicated concentration. After 18 hours, the luminescence of Firefly and Renilla luciferase were measured by Dual Luciferase Reporter Assay System (Promega E1960).

MN9D Assays. MN9Dtet-on cells were treated with 10 μM of each compound or DMSO (vehicle control) for 6 to 24 hrs. Total RNAs were isolated using Quick-RNA miniprep Plus (ZYMO Research, cat#: R¹⁰⁵⁸), and were subsequently reverse transcribed using High Capacity cDNA Reverse Transcription kit (Applied Biosystems, cat#: 4368814). SYBR Green quantitative real-time PCR analysis was performed using CFX96 Real Time system (BioRad). The following primer pairs were used for qPCR: Nurr1; 5′-CAACTACAGCACAGGCTACGA-3′ (SEQ ID NO:5) and 5′-GCATCTGAATGTCTTCTACCTTAATG-3′ (SEQ ID NO:6), Pitx3; 5′-GCAACTGGCCGCCCAAGG-3′ (SEQ ID NO:7) and 5′-AGGCCCCACGTTGACCGA-3′ (SEQ ID NO:8), VMAT2; 5′-GAAGTCCACCTGCTAAGGAAGAA-3′ (SEQ ID NO:9) and 5′-TCACTGGAGACACATGTACACAG-3′ (SEQ ID NO:10), TH; 5′-TCCAACCTTTCCTGGCCCAG-3′ (SEQ ID NO:11) and 5′-GCATGAAGGGCAGGAGGAAT-3′ (SEQ ID NO:12), HPRT; 5′-TGGGAGGCCATCACATTGT-3′ (SEQ ID NO:13) and 5′-AATCCAGCAGGTCAGCAAAGA-3′ (SEQ ID NO:14). The levels of gene expression were normalized to the level of housekeeping gene (HPRT) expression.

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All NMR spectra were recorded on a Bruker 400 (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm×4.6 mm×3.5 um), Flow Rate: 2.0 ml/min, the column temperature: 40° C.

All chemical reactions were be similarly “worked up” and then purified as follows unless otherwise noted: combined organic extracts were dried over anhydrous MgSO₄, filtered, concentrated under reduced pressure and the residue then purified by column chromatography on silica gel.

General procedure for the synthesis of hydrazides from corresponding phenoxy acetic acids, carboxylic acids, and esters. A solution of the phenoxy acetic acid (1 mmol) and hydrazine hydrate (5 mmol) in EtOH was refluxed for 18-24 h. Solvents were removed under reduced pressure and crude products were extracted with CH₂Cl₂ or EtOAc.

General procedure for the synthesis of aryl pyrazoles from the corresponding boronic acids and halo-pyrazoles. A solution of the commercial heteroarylboronic acid (1.1 equiv), [Pd₂(dba)₃] (0.010 equiv), and PCy₃ₙ (0.024 equiv) were added to a Schlenk flask equipped with a stir bar in air. The flask was evacuated and refilled with argon five times. Dioxane, the (hetero)aryl halide (1.0 equiv; if the halide is a solid, it was added prior to the evacuation/refill cycle), and aqueous K₃PO₄ (1.27 M, 1.70 equiv) were added by syringe. The Schlenk flask was sealed and heated in an oil bath at 100° C. for 18 h with vigorous stirring. The mixture was then filtered through a pad of silica gel (washing with EtOAc), the filtrate concentrated under reduced pressure, and the aqueous residue extracted three times with EtOAc.

General procedure for synthesis of acrylamide analogs from amines (hydrazines, pyrazols). To an ice-cold solution of the amine (1.0 equiv) in anhydrous EtOAc was added Et₃N (1.5 equiv), followed by acryloyl chloride (1.2 eqiuv). The resulting mixture was allowed to warm to ambient temperature, and stirred for ˜2 h. Upon complete consumption of amine, the reaction mixture was diluted with water, then extracted with EtOAc.

General procedure for the synthesis of sulfonamide analogs from amines (hydrazines, pyrazoles). To a solution of the amine (1.0 equiv) and DMAP (0.1 equiv) in CH₂Cl₂ was added Et₃N (3.0 equiv). The mixture was stirred under argon for several minutes until the materials were dissolved and then cooled to 0° C. Next, 2-chloroethanesulfonyl chloride (1.4 equiv) was added drop-wise over several minutes. Upon complete consumption of amine, the reaction mixture was diluted with water, then extracted with CH₂Cl₂.

General procedure for the synthesis of alkyl chloride analogs from amines (hydrazines, pyrazoles). To an ice-cold solution of the amine (1.0 equiv) in anhydrous CH₂Cl₂, was added Et₃N (1.5 equiv) followed by acryloyl chloride (1.2 eqiuv). The mixture was allowed to warm to ambient temperature and stirred for 2 h. Upon complete consumption of amine, the reaction mixture was diluted with water, then extracted with CH₂Cl₂.

SU20666-0001

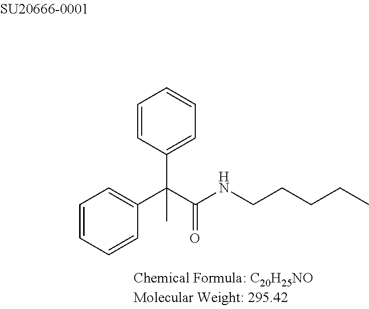 [SMILES: CCCCCNC(=O)C(C)(c1ccccc1)c1ccccc1]

Route for SU20666-0001

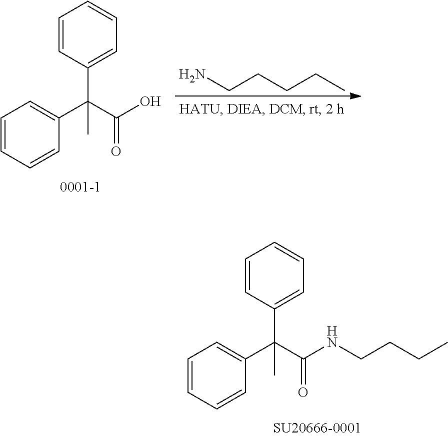

The synthesis of N-pentyl-2,2-diphenylpropanamide (SU20666-0001).

To a stirred solution of 0001-1 (200 mg, 0.88 mmol) in DCM (10 ml) was added pentan-1-amine (92 mg, 1.06 mmol), DIEA (342 mg, 2.66 mmol) and HATU (504 mg, 1.33 mmol). The resulting reaction mixture was stirred at rt for 2 h. Then added water, the aqueous phase was extracted with dichloromethane, the combined organic phases were dried over anhydrous sodium sulfate, filtered, and concentrated in vacuo-purified by prep-HPLC to give the desired product SU20666-0001 (80 mg, yield: 31%) as white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.220 min; MS Calcd.: 295.2; MS Found: 296.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.484 min.

¹H NMR (400 MHz, DMSO-d₆) δ 0.83 (3H, t, J=6.8 Hz), 1.12-1.25 (4H, m), 1.37-1.41(2H, m), 1.84 (3H, s), 3.07 (2H, q, J=6.8 Hz), 7.15-7.17 (4H, m), 7.21-7.32 (7H, m).

SU20666-0002

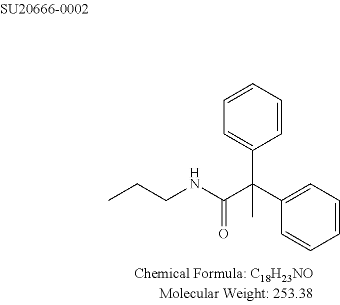 [SMILES: CCCNC(=O)C(C)(c1ccccc1)c1ccccc1]

Route for SU20666 0002

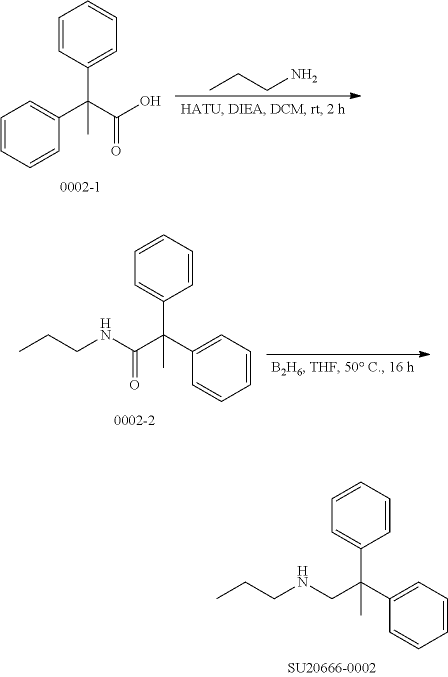

The synthesis of 2,2-diphenyl-N-propylpropanamide (SU20666-0002-2).

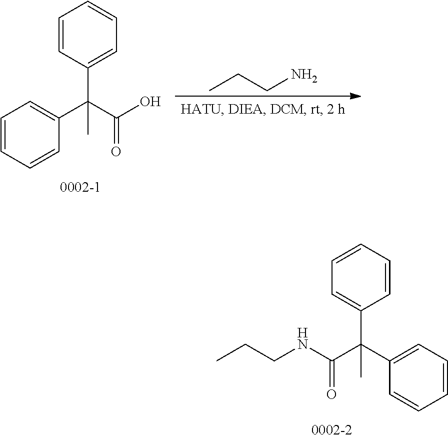

To a stirred solution of 0002-1 (500 mg, 2.2 mmol) in DCM (10 ml) was added propan-1-amine (157 mg, 2.6 mmol), DIEA (851 mg, 6.6 mmol) and HATU (1250 mg, 3.3 mmol). The resulting reaction mixture was stirred at rt for 2 h. Then added water, the aqueous phase was extracted with dichloromethane, the combined organic phases were dried over anhydrous sodium sulfate, filtered, and concentrated in vacuopurified by prep-HPLC to give the desired product SU20666-0002-2 (415 mg, yield: 70%) as white solid.

The synthesis of 2,2-diphenyl-N-propylpropanamide (SU20666-0002).

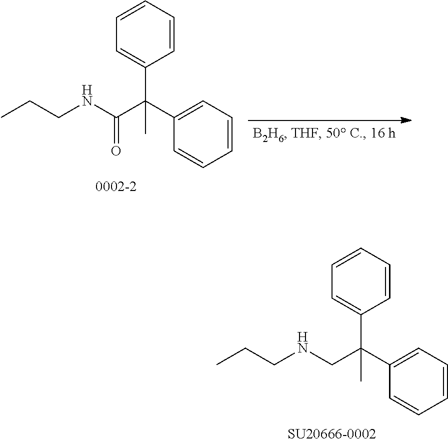 [SMILES: CCCNC(=O)C(C)(c1ccccc1)c1ccccc1 ; CCCNCC(C)(c1ccccc1)c1ccccc1]

To a stirred solution of 0002-2 (200 mg, 0.75 mmol) in THF (10 ml) was added borane-tetrahydrofuran (1.0 N, 4.5 mL, 4.5 mmol). The resulting reaction mixture was heated to 50° C. and stirred for 16 h. Then added HCl (1.0 N, 3 mL) and stirred for 1 h at rt, the aqueous phase was neutralized and then extracted with dichloromethane, the combined organic phases were dried over anhydrous sodium sulfate, filtered, and concentrated in vacuo, the crude was purified by prep-HPLC to give the desired product SU20666-0002 (15 mg, yield: 7.9%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.01%, Rt=2.786 min; MS Calcd.: 253.2; MS Found: 254.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.17%, Rt=11.992 min.

¹H NMR (400 MHz, CDCl₃) δ 0.83 (3H, t, J=7.2 Hz), 1.39-1.42 (3H, m), 1.74 (3H, s), 2.54 (2H, t, J=6.8 Hz), 3.20 (2H, s), 7.16-7.22 (6H, m), 7.27-7.30 (4H, m).

SU20666-0003

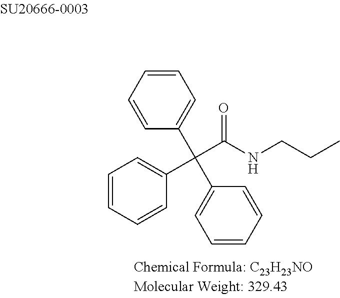 [SMILES: CCCNC(=O)C(c1ccccc1)(c1ccccc1)c1ccccc1]

Route for SU20666-0003

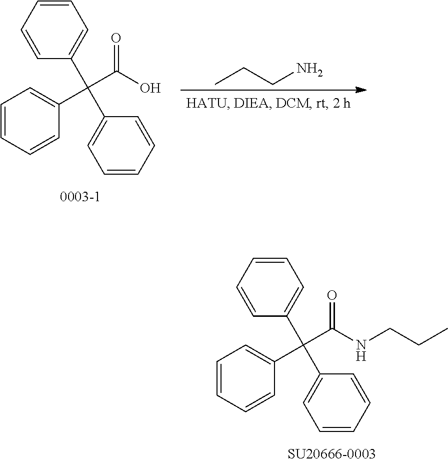

The synthesis of 2,2,2-triphenyl-N-propylacetamide (SU20666-0003).

To a stirred solution of 0003-1 (200 mg, 0.7 mmol) in DCM (10 ml) was added propan-1-amine (49 mg, 0.83 mmol), DIEA (271 mg, 2.1 mmol) and HATU (400 mg, 1.1 mmol). The resulting reaction mixture was stirred at rt for 2 h. Then added water, the aqueous phase was extracted with dichloromethane, the combined organic phases were dried over anhydrous sodium sulfate, filtered, and concentrated in vacuo, purified by prep-HPLC to give the desired product SU20666-0003 (160 mg, yield: 70%) as white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.245 min; MS Calcd.: 329.2; MS Found: 330.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.587 min.

¹H NMR (400 MHz, DMSO-d₆) δ 0.71 (3H, t, J=7.2 Hz), 1.34-1.40 (2H, m), 3.08 (2H, q, J=6.8 Hz), 7.08 (1H, t, J=5.6 Hz), 7.19-7.31 (15H, m).

SU20666-0004

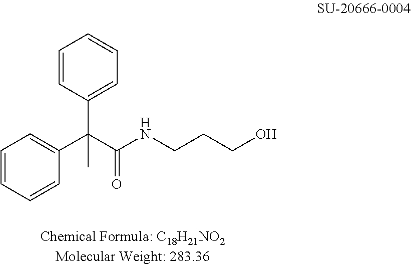 [SMILES: CC(C(=O)NCCCO)(c1ccccc1)c1ccccc1]

Route for SU20666-0004

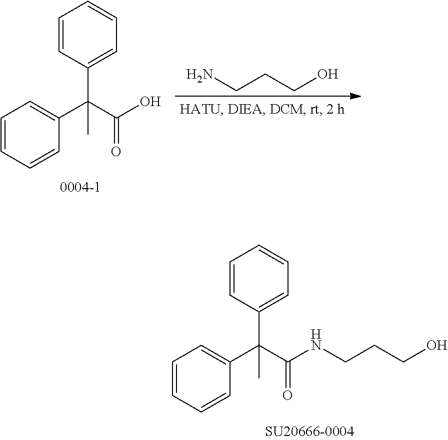 [SMILES: CC(C(=O)O)(c1ccccc1)c1ccccc1 ; NCCCO ; CC(C(=O)NCCCO)(c1ccccc1)c1ccccc1]

The synthesis of N-(3-hydroxypropyl)-2,2-diphenylpropanamide (SU20666-0004).

To a stirred solution of 0004-1 (200 mg, 0.88 mmol) in DCM (10 ml) was added 3-aminopropan-1-ol (80 mg, 1.1 mmol), DIEA (342 mg, 2.7 mmol) and HATU (504 mg, 1.3 mmol). The resulting reaction mixture was stirred at rt for 2 h. Then added water, the aqueous phase was extracted with dichloromethane, the combined organic phases were dried over anhydrous sodium sulfate, filtered, and concentrated in vacuo, purified by prep-HPLC to give the desired product SU20666-0004 (110 mg, yield: 44%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.656 min; MS Calcd.: 283.2; MS Found: 284.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.327 min.

¹H NMR (400 MHz, DMSO-d₆) δ 1.51-1.58 (2H, m), 1.84 (3H, s), 3.15 (2H, q, J=6.8 Hz), 3.35-3.37 (2H, m), 4.38 (1H, t, J=4.8 Hz), 7.15-7.17 (4H, m), 7.21-7.32 (7H, m).

SU20666-0005

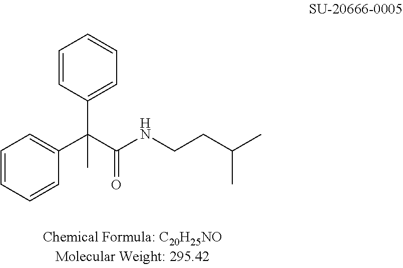 [SMILES: CC(C)CCNC(=O)C(C)(c1ccccc1)c1ccccc1]

Route for SU20666-0005

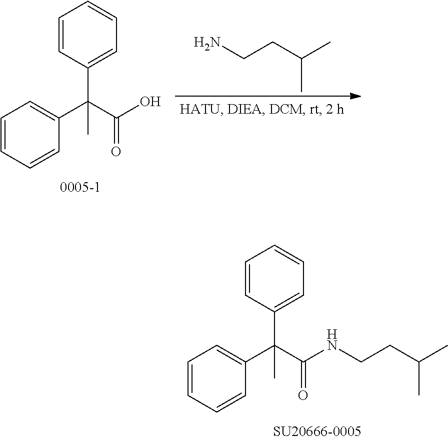

The synthesis of N-isopentyl-2,2-diphenylpropanamide (SU20666-0005).

To a stirred solution of 0005-1 (200 mg, 0.88 mmol) in DCM (10 ml) was added 3-methylbutan-1-amine (92 mg, 1.1 mmol), DIEA (342 mg, 2.7 mmol) and HATU (504 mg, 1.3 mmol). The resulting reaction mixture was stirred at rt for 2 h. Then added water, the aqueous phase was extracted with dichloromethane, the combined organic phases were dried over anhydrous sodium sulfate, filtered, and concentrated in vacuo, purified by prep-HPLC to give the desired product SU20666-0005 (150 mg, yield: 57%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.208 min; MS Calcd.: 295.2; MS Found: 296.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.413 min.

¹H NMR (400 MHz, DMSO-d₆) δ 0.83 (6H, d, J=6.4 Hz), 1.28 (2H, q, J=7.2 Hz), 1.43-1.50 (1H, m), 1.84 (3H, s), 3.10 (2H, q, J=6.4 Hz), 7.14-7.16 (4H, m), 7.21-7.31 (7H, m).

SU20666-0006

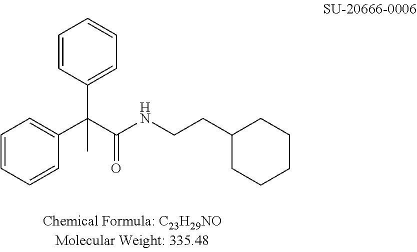 [SMILES: CC(C(=O)NCCC1CCCCC1)(c1ccccc1)c1ccccc1]

Route for SU20666-0006

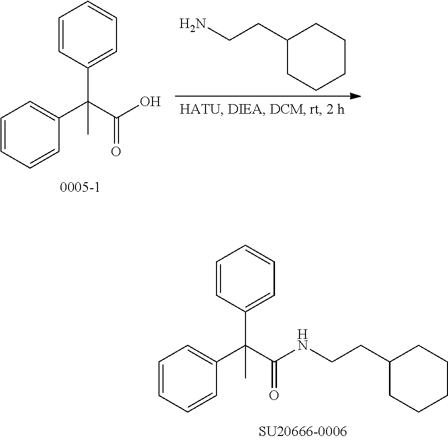 [SMILES: CC(C(=O)O)(c1ccccc1)c1ccccc1 ; NCCC1CCCCC1 ; CC(C(=O)NCCC1CCCCC1)(c1ccccc1)c1ccccc1]

The synthesis of N-(2-cyclohexylethyl)-2,2-diphenylpropanamide (SU20666-0006).

To a stirred solution of 0006-1 (200 mg, 0.88 mmol) in DCM (10 ml) was added 2-cyclohexylethanamine (174 mg, 1.1 mmol), DIEA (342 mg, 2.7 mmol) and HATU (504 mg, 1.3 mmol). The resulting reaction mixture was stirred at rt for 2 h. Then added water, the aqueous phase was extracted with dichloromethane, the combined organic phases were dried over anhydrous sodium sulfate, filtered, and concentrated in vacuo, purified by prep-HPLC to give the desired product SU20666-0006 (118 mg, yield: 40%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 94.91%, Rt=2.406 min; MS Calcd.: 335.2; MS Found: 336.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 94.40%, Rt=12.424 min.

¹H NMR (400 MHz, DMSO-d₆) δ 0.77-0.85 (2H, m), 1.06-1.15 (2H, m), 1.28 (2H, q, J=6.8 Hz), 1.61-1.63 (5H, m), 1.83 (3H, s), 3.11 (2H, q, J=6.8 Hz), 7.15-7.17 (4H, m), 7.21-7.25 (3H, m), 7.28-7.32 (4H, m).

SU20666-0015

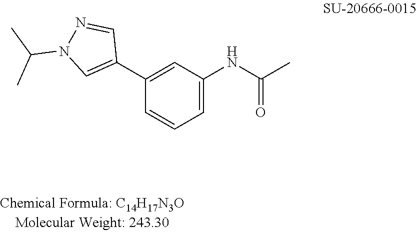 [SMILES: CC(=O)Nc1cccc(-c2cnn(C(C)C)c2)c1]

Route for 0015-2

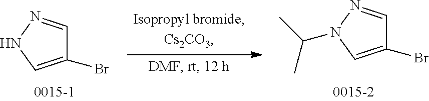

To a stirred solution of 0015-1 (1.0 g, 6.8 mmol) in DMF (20 ml) was added isopropyl bromide (878 mg, 7.2 mmol), Cs₂CO₃ (3.3 g, 10.2 mmol). The resulting reaction mixture was stirred at rt for 12 h. Then added water, the aqueous phase was extracted with dichloromethane, the combined organic phases were dried over anhydrous sodium sulfate, filtered, and concentrated in vacuo, further purified by C.C. to give the desired product 0015-2 (1.1 g, yield: 85%) as colorless oil.

Route for SU20666-0015

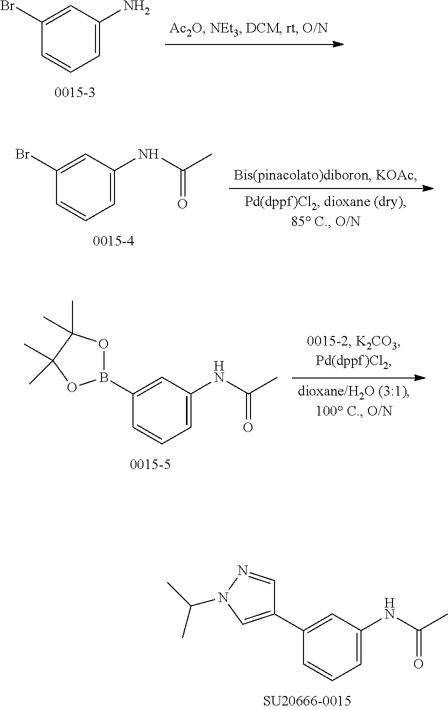 [SMILES: Nc1cccc(Br)c1 ; CC(=O)Nc1cccc(Br)c1 ; CC(=O)Nc1cccc(B2OC(C)(C)C(C)(C)O2)c1 ; CC(=O)Nc1cccc(-c2cnn(C(C)C)c2)c1]

The synthesis of N-(3-bromophenyl)acetamide (0015-4).

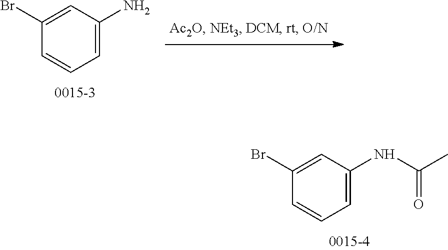

To a stirred solution of 0015-3 (16.0 g, 93.6 mmol) in DCM (200 ml) was added TEA (11.5 g, 112 mmol) and Ac₂O (11.5 g, 112 mmol). The resulting reaction mixture was stirred at rt for 12 h. Then added water, the aqueous phase was extracted with dichloromethane, the combined organic phases were dried over anhydrous sodium sulfate, filtered, and concentrated to give the desired product 0015-4 (10 g, yield: 53%) as a yellow solid.

The synthesis of N-(3-(4,4,5,5-tetramethyl-1,3,2-dioxaborolan-2-yl)phenyl)acetamide (0015-5).

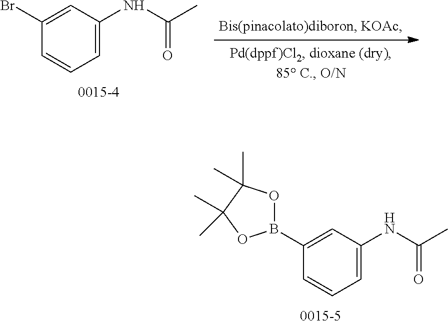 [SMILES: CC(=O)Nc1cccc(Br)c1 ; CC(=O)Nc1cccc(B2OC(C)(C)C(C)(C)O2)c1]

To a stirred solution of compound 3-bromo-5-chloro-1,2,4-thiadiazole (0015-4, 10.3 g, 48.4 mmol) in dioxane (200mL) was added bis(pinacolato)diboron (18.4 g, 72.5 mmol), KOAc (14.2 g, 145.2 mmol), Pd(dppf)Cl₂ (1.7 g, 2.42 mmol). The resulting reaction mixture was heated to 85° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 0015-5 (5.0 g, yield: 40%) as a yellow solid.

The synthesis of N-(3-(1-isopropyl-1H-pyrazol-4-yl)phenyl)acetamide (SU20666-0015).

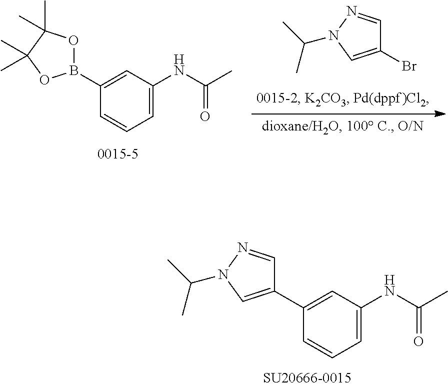

To a stirred solution of compound 0015-5 (206 mg, 1.1 mmol) in dioxane/water (10 mL/2 mL) was added 0015-2 (226 mg, 1.2 mmol), K₂CO₃ (451 mg, 3.3 mmol), Pd(dppf)Cl₂ (73 mg, 0.10 mmol). The resulting reaction mixture was heated to 100° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0015 (100 mg, yield: 38%) as a yellow solid.

LC-MS (LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min.), Purity: 100%, Rt=0.562 min; MS Calcd.: 243.1; MS Found: 244.3 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 94.40%, Rt=7.188 min.

¹H NMR (400 MHz, DMSO-d₆) δ 1.44 (6H, d, J=6.8 Hz), 2.05 (3H, s), 4.48-4.55 (1H, m), 7.22-7.28 (2H, m), 7.38-7.40 (1H, m), 7.74 (2H, s), 8.10 (1H, s), 9.92 (1H, s).

SU20666-0016

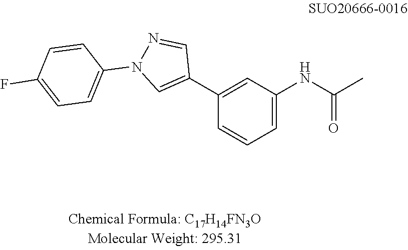 [SMILES: CC(=O)Nc1cccc(-c2cnn(-c3ccc(F)cc3)c2)c1]

Route for SU20666-0016

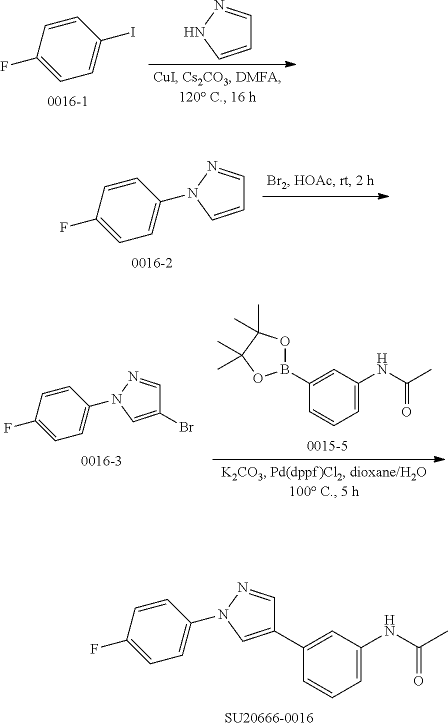 [SMILES: Fc1ccc(I)cc1 ; c1cn[nH]c1 ; Fc1ccc(-n2cccn2)cc1 ; Fc1ccc(-n2cc(Br)cn2)cc1 ; CC(=O)Nc1cccc(B2OC(C)(C)C(C)(C)O2)c1 ; CC(=O)Nc1cccc(-c2cnn(-c3ccc(F)cc3)c2)c1]

The synthesis of 1-(4-fluorophenyl)-1H-pyrazole (0016-2).

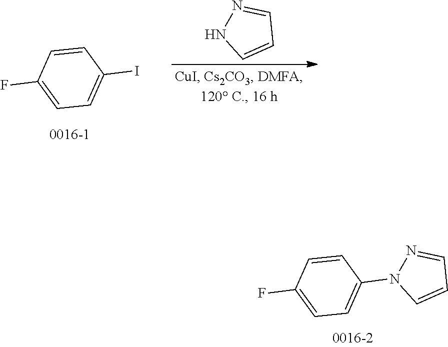 [SMILES: Fc1ccc(I)cc1 ; c1cn[nH]c1 ; Fc1ccc(-n2cccn2)cc1]

To a stirred solution of 0016-1 (6.5 g, 29 mmol) in DMFA (50 ml) was added 1H-pyrazole (2.0 g, 29 mmol), Cs₂CO₃ (11.3 g, 35 mmol) and CuI (0.55 g, 2.9 mmol). The resulting reaction mixture was heated to 120° C. for 16 h. Then added water, the aqueous phase was extracted with dichloromethane, the combined organic phases were dried over anhydrous sodium sulfate, filtered, and concentrated in vacuo, thus was further purified by C.C. to give the desired product 0016-2 (3.6 g, yield: 75%) as yellow oil.

The synthesis of 4-bromo-1-(4-fluorophenyl)-1H-pyrazole (0016-3).

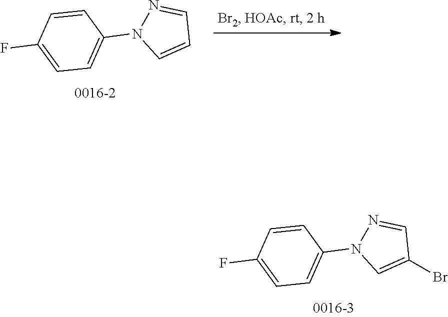 [SMILES: Fc1ccc(-n2cccn2)cc1 ; Fc1ccc(-n2cc(Br)cn2)cc1]

To a stirred solution of 0016-2 (0.50 g, 3.1 mmol) in HOAc (10 ml) was added Br₂ (1.0 g, 6.2 mmol) slowly. The resulting reaction mixture was stirred at rt for 2 h. Then added water, the aqueous phase was extracted with dichloromethane, the combined organic phases were dried over anhydrous sodium sulfate, filtered and concentrated to give the desired product 0016-3 (0.70 g, yield: 94%) as a yellow solid.

The synthesis of N-(3-(1-(4-fluorophenyl)-1H-pyrazol-4-yl)phenypacetamide (SU20666-0016).

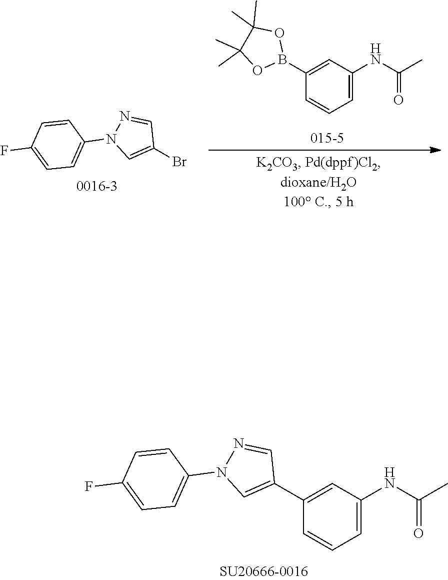 [SMILES: CC(=O)Nc1cccc(B2OC(C)(C)C(C)(C)O2)c1 ; Fc1ccc(-n2cc(Br)cn2)cc1 ; CC(=O)Nc1cccc(-c2cnn(-c3ccc(F)cc3)c2)c1]

To a stirred solution of compound 0016-3 (200 mg, 0.83 mmol) in dioxane/water (10 mL/2 mL) was added 015-5 (220 mg, 0.83 mmol), K₂CO₃ (140 mg, 0.99 mmol), Pd(dppf)Cl₂ (50 mg). The resulting reaction mixture was heated to 100° C. and stirred for 5 h and concentrated in vacuo to remove the solvent, then added water, the aqueous phase was extracted with dichloromethane, the combined organic phases was dried over anhydrous sodium sulfate, filtered, and concentrated, the crude was purified by prep-HPLC to give the desired product SU20666-0016 (60 mg, yield: 25%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.973 min; MS Calcd.: 295.1; MS Found: 296.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=9.391 min.

¹H NMR (400 MHz, DMSO-d₆) δ 2.06 (3H, s), 7.31-7.45 (5H, m), 7.85 (1H, s), 7.92-7.96 (2H, m), 8.08 (1H, s), 8.89 (1H, s), 9.97 (1H, s).

SU20666-0017 and SU20666-0057

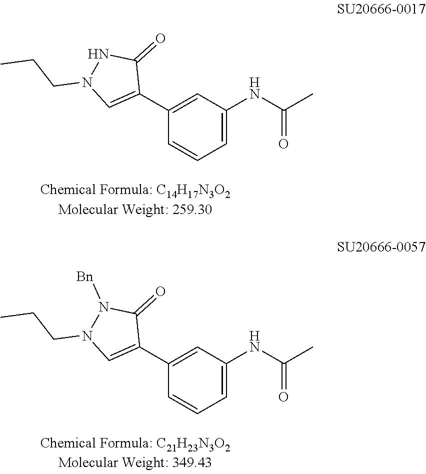

Route for SU20666-0017

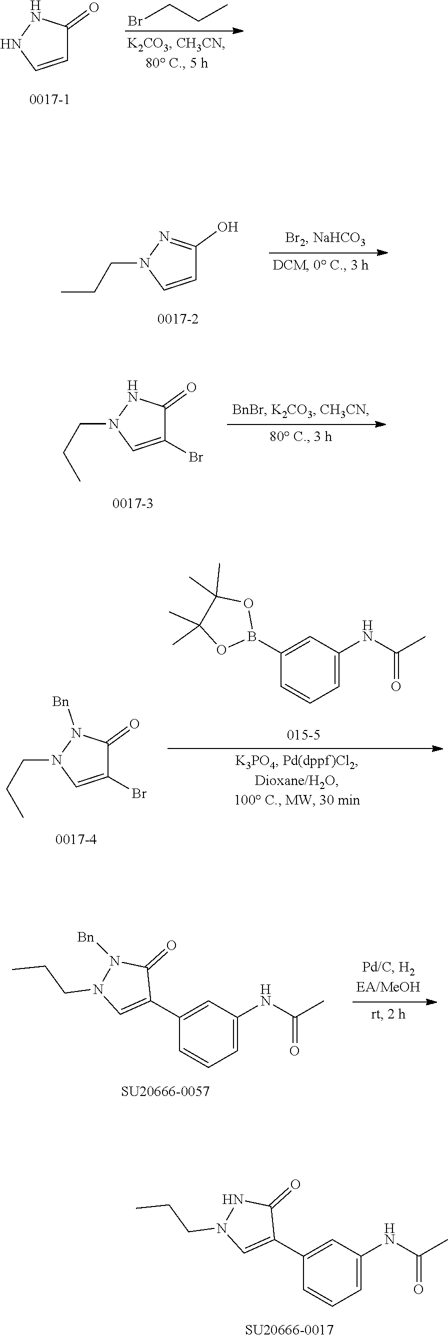

The synthesis of 1-propyl-1H-pyrazol-3-ol (0017-2).

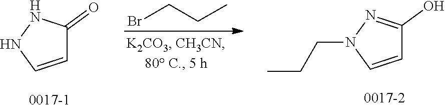

To a stirred solution of 0017-1 (1.5 g, 17.9 mmol) in CH₃CN (50 ml) was added 1-bromopropane (2.2 g, 17.9 mmol), K₂CO₃ (2.7 g, 19.6 mmol). The resulting reaction mixture was heated to 80° C. for 5 h. Then added water, the aqueous phase was extracted with dichloromethane, the combined organic phases were dried over anhydrous sodium sulfate, filtered, and concentrated in vacuopurified by C.C. to give the desired product 0017-2 (0.40 g, yield: 18%) as a yellow solid.

The synthesis of 4-bromo-1-propyl-1H-pyrazol-3(2H)-one (0017-3).

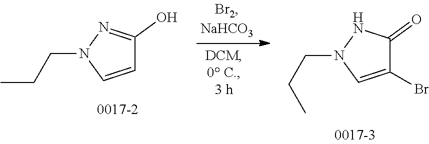

To a stirred solution of 0017-2 (0.30 g, 2.4 mmol) in DCM (20 ml) was added NaHCO₃ (0.24 g, 2.8 mmol) and Br₂ (0.42 g, 2.6 mmol) slowly. The resulting reaction mixture was stirred at 0° C. for 3 h. Then added water, the aqueous phase was extracted with dichloromethane, the combined organic phases were dried over anhydrous sodium sulfate, filtered, and concentrated to give the desired product 0017-3 (0.40 g, yield: 82%) as a yellow solid.

The synthesis of 2-benzyl-4-bromo-1-propyl-1H-pyrazol-3(2H)-one (0017-4).

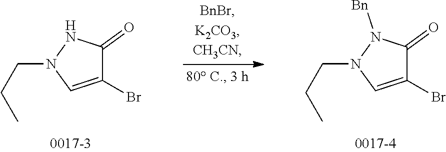

To a stirred solution of 0017-3 (0.30 g, 1.46 mmol) in CH₃CN (20 ml) was added K₂CO₃ (0.22 g, 1.6 mmol) and BnBr (0.28 g, 1.6 mmol). The resulting reaction mixture was heated to 80° C. for 3 h. Then added water, the aqueous phase was extracted with dichloromethane, the combined organic phases were dried over anhydrous sodium sulfate, filtered, and concentrated to give the desired product 0017-4 (0.38 g, yield: 88%) as a yellow solid.

The synthesis of N-(3-(2-benzyl-3-oxo-1-propyl-2,3-dihydro-1H-pyrazol-4-yl)phenyl)acetamide (SU20666-0057).

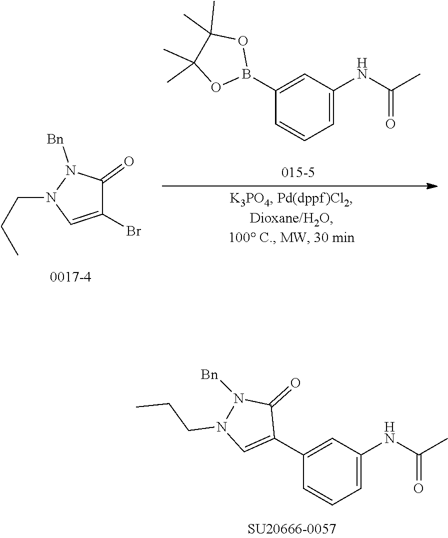 [SMILES: CCCn1cc(Br)c(=O)n1Cc1ccccc1 ; CC(=O)Nc1cccc(B2OC(C)(C)C(C)(C)O2)c1 ; CCCn1cc(-c2cccc(NC(C)=O)c2)c(=O)n1Cc1ccccc1]

To a solution of compound 0017-4 (115 mg, 0.39 mmol) in dioxane/water (5 mL/1 mL) was added 015-5 (112 mg, 0.43 mmol), K₃PO₄ (155 mg, 0.58 mmol), Pd(dppf)Cl₂ (20 mg). The resulting reaction mixture was heated to 100° C. and stirred for 0.5 h at MW conditions, then concentrated in vacuo to remove the solvent and added water, the aqueous phase was extracted with dichloromethane, the combined organic phases were dried over anhydrous sodium sulfate, filtered, and concentrated, the crude was purified by prep-TLC to give the desired product SU20666-0057 (30 mg, yield: 22%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.13%, Rt=2.060 min; MS Calcd.: 349.2; MS Found: 350.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.13%, Rt=10.126 min.

¹H NMR (400 MHz, CDCl₃) δ 0.86 (3H, t, J=7.6 Hz), 1.78-1.83 (2H, m), 2.10 (3H, s), 3.87 (2H, t, J=6.8 Hz), 5.28 (2H, s), 7.02 (1H, s), 7.19-7.36 (6H, m), 7.42-7.44 (3H, m), 7.66 (1H, s).

The synthesis of N-(3-(3-oxo-1-propyl-2,3-dihydro-1H-pyrazol-4-yl)phenyl)acetamide (SU20666-0017).

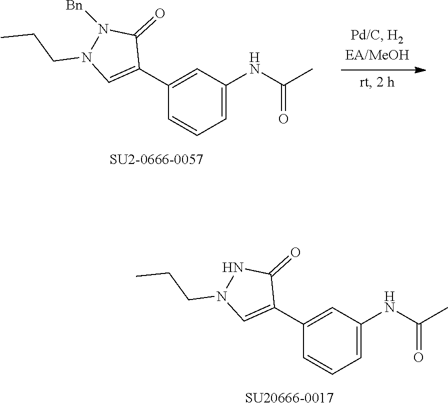

To a stirred solution of compound SU20666-0057 (30 mg, 0.086 mmol) in EA/methanol (10 mL/2 mL) was added Pd/C (10%, 10 mg). The resulting reaction mixture was stirred for 2 h at rt and filtered, the filtrate was concentrated in vacuo to remove the solvent and further purified by prep-HPLC to give the desired product SU20666-0017 (5 mg, yield: 23%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.24%, Rt=1.399 min; MS Calcd.: 259.1; MS Found: 260.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 98.83%, Rt=6.745 min.

¹H NMR (400 MHz, DMSO-d₆) δ 0.84 (3H, t, J=7.2 Hz), 1.73-1.78 (2H, m), 2.02 (3H, s), 3.85 (2H, t, J=6.8 Hz), 7.17-7,21 (1H, m), 7.25-7.27 (1H, m), 7.40-7.42 (1H, d, J=9.2 Hz), 7.78-7.82 (2H, m), 9.86 (1H, s), 10.24 (1H, s).

SU20666-0018

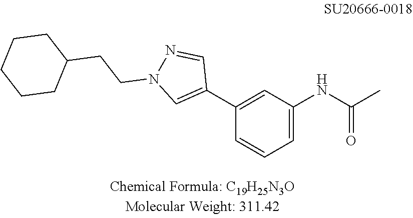 [SMILES: CC(=O)Nc1cccc(-c2cnn(CCC3CCCCC3)c2)c1]

Route for SU20666-0018

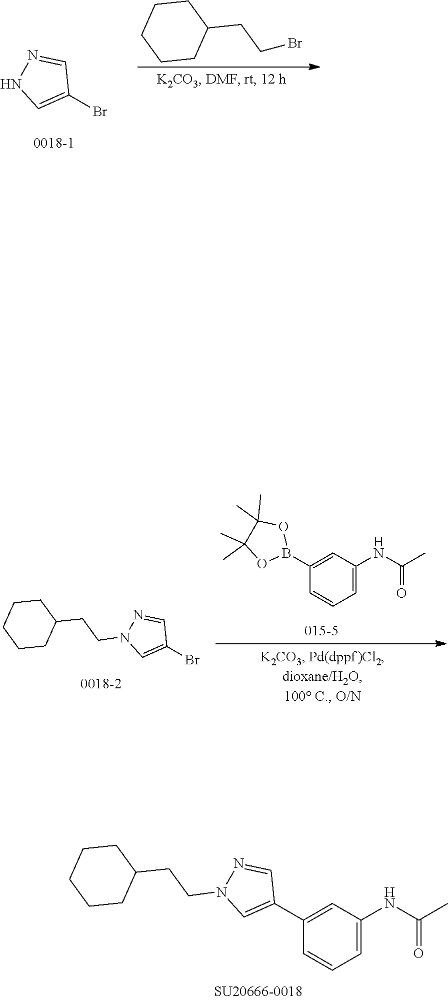 [SMILES: Brc1cn[nH]c1 ; BrCCC1CCCCC1 ; Brc1cnn(CCC2CCCCC2)c1 ; CC(=O)Nc1cccc(B2OC(C)(C)C(C)(C)O2)c1 ; CC(=O)Nc1cccc(-c2cnn(CCC3CCCCC3)c2)c1]

The synthesis of 4-bromo-1-(2-cyclohexylethyl)-1H-pyrazole (0018-2).

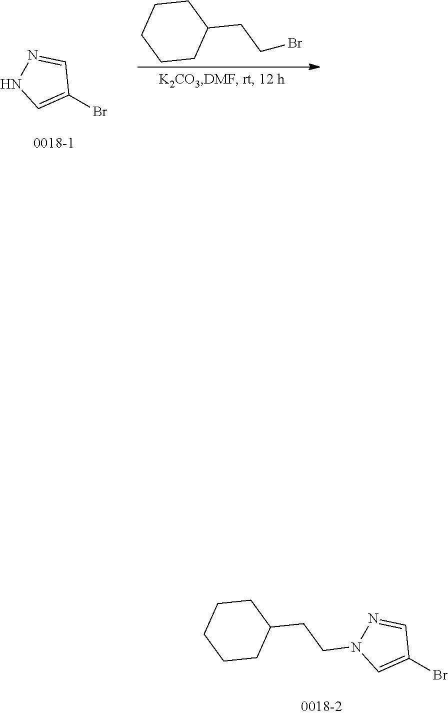 [SMILES: Brc1cn[nH]c1 ; BrCCC1CCCCC1 ; Brc1cnn(CCC2CCCCC2)c1]

To a stirred solution of 0018-1 (0.5 g, 3.4 mmol) in DMF (10 ml) was added (2-bromoethyl)cyclohexane (0.78 g, 4.1 mmol) and K₂CO₃ (0.94 g, 6.8 mmol). The resulting reaction mixture was stirred for 12 h at rt. Then added water, the aqueous phase was extracted with dichloromethane, the combined organic phases were dried over anhydrous sodium sulfate, filtered, and concentrated in vacuo, purified by C.C. to give the desired product 0018-2 (0.80 g, yield: 92%) as colorless oil.

The synthesis of N-(3-(1-(2-cyclohexylethyl)-1H-pyrazol-4-yl)phenyl)acetamide (SU20666-0018).

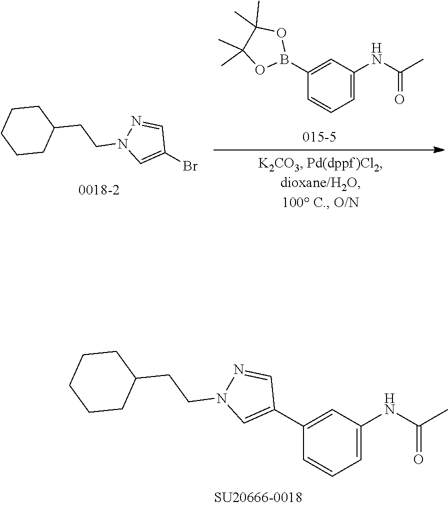 [SMILES: Brc1cnn(CCC2CCCCC2)c1 ; CC(=O)Nc1cccc(B2OC(C)(C)C(C)(C)O2)c1 ; CC(=O)Nc1cccc(-c2cnn(CCC3CCCCC3)c2)c1]

To a stirred solution of compound 0018-2 (366 mg, 1.4 mmol) in dioxane/water (10 mL/2 mL) was added 0015-5 (300 mg, 1.2 mmol), K₂CO₃ (322 mg, 2.3 mmol), Pd(dppf)Cl₂ (30 mg). The resulting reaction mixture was heated to 100° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0018 (25 mg, yield: 7%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.43%, Rt=2.462 min; MS Calcd.: 311.2; MS Found: 312.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.82%, Rt=10.172 min.

¹H NMR (400 MHz, DMSO-d₆) δ 0.93-1.02 (2H, q, J=7.2 Hz), 1.13-1.31 (4H, m), 1.63-1.82 (7H, m), 2.20 (3H, s), 4.16 (2H, t, J=7.6 Hz), 7.19-7,22 (2H, m), 7.28-7.30 (2H, m), 7.63 (1H, s), 7.73-7.75 (2H, m).

SU20666-0019

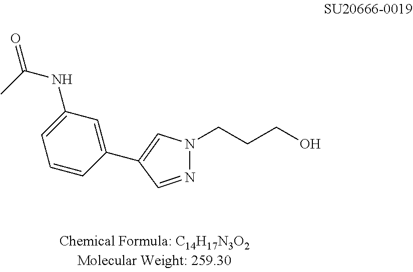 [SMILES: CC(=O)Nc1cccc(-c2cnn(CCCO)c2)c1]

Route for SU20666-0019

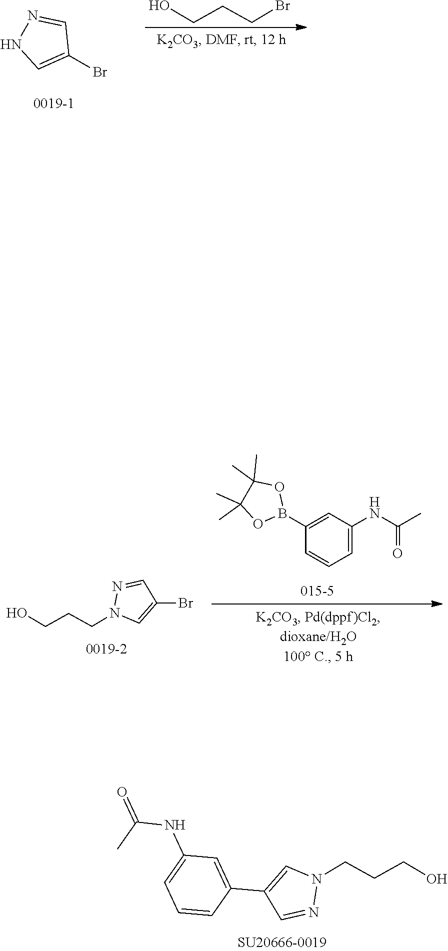 [SMILES: Brc1cn[nH]c1 ; OCCCBr ; CC(=O)Nc1cccc(B2OC(C)(C)C(C)(C)O2)c1 ; OCCCn1cc(Br)cn1 ; CC(=O)Nc1cccc(-c2cnn(CCCO)c2)c1]

The synthesis of 3-(4-bromo-1H-pyrazol-1-yl)propan-1-ol (0019-2).

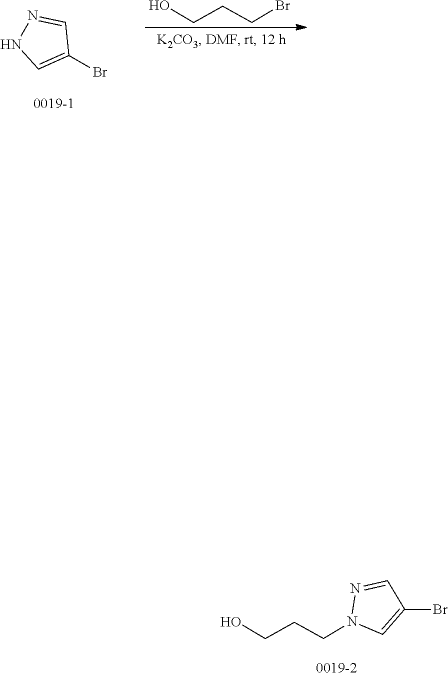 [SMILES: Brc1cn[nH]c1 ; OCCCBr ; OCCCn1cc(Br)cn1]

To a stirred solution of 0019-1 (2.7 g, 18.2 mmol) in DMF (50 ml) was added 3-bromopropan-1-ol (2.8 g, 20.0 mmol) and K₂CO₃ (3.8 g, 27.3 mmol). The resulting reaction mixture was stirred for 12 h at rt. Then added water, the aqueous phase was extracted with dichloromethane, the combined organic phases were dried over anhydrous sodium sulfate, filtered, and concentrated in vacuo, purified by C.C. to give the desired product 0019-2 (1.7 g, yield: 46%) as colorless oil.

The synthesis of N-(3-(1-(3-hydroxypropyl)-1H-pyrazol-4-yl)phenyl)acetamide (SU20666-0019).

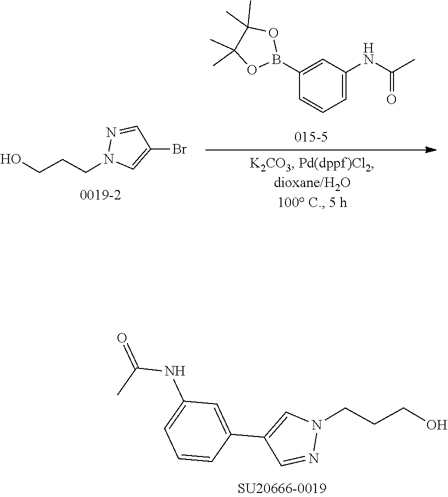 [SMILES: CC(=O)Nc1cccc(B2OC(C)(C)C(C)(C)O2)c1 ; OCCCn1cc(Br)cn1 ; CC(=O)Nc1cccc(-c2cnn(CCCO)c2)c1]

To a stirred solution of compound 0019-2 (240 mg, 1.2 mmol) in dioxane/water (10 mL/2 mL) was added 0015-5 (305 mg, 1.2 mmol), K₂CO₃ (484 mg, 3.5 mmol), Pd(dppf)Cl₂ (80 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0019 (49 mg, yield: 16%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.43%, Rt=1.389 min; MS Calcd.: 259.1; MS Found: 260.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.82%, Rt=6.418 min.

¹H NMR (400 MHz, DMSO-d₆) δ 1.92-1.97 (2H, m), 2.05 (3H, s), 3.40-3.42 (2H, m), 4.18 (2H, t, J=6.8 Hz), 4.62 (1H, t, J=5.2 Hz), 7.21-7.28 (2H, m), 7.38 (1H, d, J=7.2 Hz), 7.74-7.76 (2H, m), 8.07 (1H, s), 9.93 (1H, s).

SU20666-0020

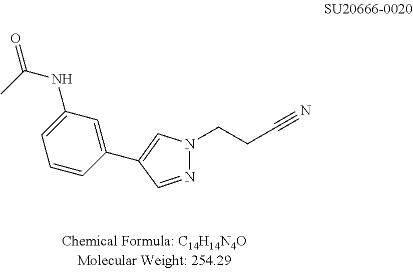 [SMILES: CC(=O)Nc1cccc(-c2cnn(CCC#N)c2)c1]

Route for SU20666-0020

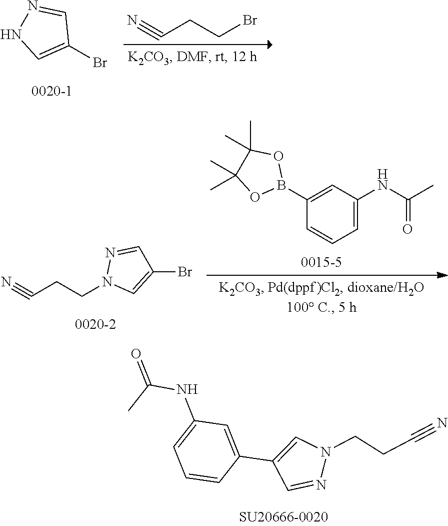 [SMILES: Brc1cn[nH]c1 ; N#CCCBr ; CC(=O)Nc1cccc(B2OC(C)(C)C(C)(C)O2)c1 ; N#CCCn1cc(Br)cn1 ; CC(=O)Nc1cccc(-c2cnn(CCC#N)c2)c1]

The synthesis of 3-(4-bromo-1H-pyrazol-1-yl)propanenitrile (0020-2).

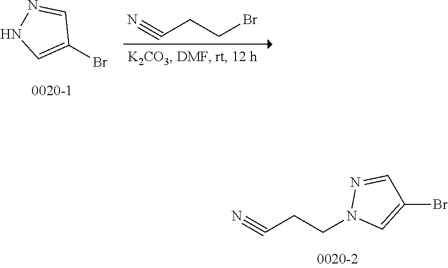 [SMILES: Brc1cn[nH]c1 ; N#CCCBr ; N#CCCn1cc(Br)cn1]

To a stirred solution of 0020-1 (1.5 g, 10.2 mmol) in DMF (20 ml) was added 3-bromopropanenitrile (1.6 g, 12.2 mmol) and K₂CO₃ (2.8 g, 20.4 mmol). The resulting reaction mixture was stirred for 12 h at rt. Then added water, the aqueous phase was extracted with dichloromethane, the combined organic phases were dried over anhydrous sodium sulfate, filtered, and concentrated in vacuo to give the desired product 0020-2 (1.9 g, yield: 92%) as yellow oil.

The synthesis of N-(3-(1-(2-cyanoethyl)-1H-pyrazol-4-yl)phenyl)acetamide (SU20666-0020).

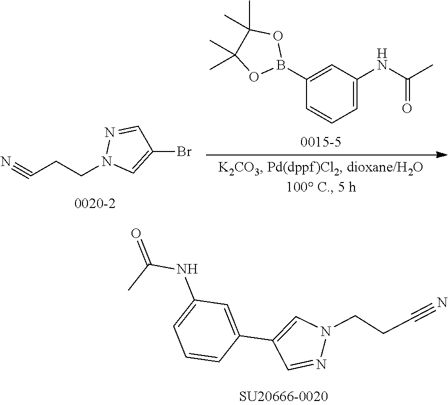 [SMILES: CC(=O)Nc1cccc(B2OC(C)(C)C(C)(C)O2)c1 ; N#CCCn1cc(Br)cn1 ; CC(=O)Nc1cccc(-c2cnn(CCC#N)c2)c1]

To a stirred solution of compound 0020-2 (200 mg, 1.0 mmol) in dioxane/water (10 mL/2 mL) was added 0015-5 (260 mg, 1.0 mmol), K₂CO₃ (210 mg, 1.5 mmol), Pd(dppf)Cl₂ (50 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0020 (20 mg, yield: 8%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.81%, Rt=1.382 min; MS Calcd.: 254.1; MS Found: 255.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 93.00%, Rt=6.633 min.

¹H NMR (400 MHz, DMSO-d₆) δ 2.05 (3H, s), 3.20 (2H, t, J=6.4 Hz), 4.42 (2H, t, J=6.4 Hz), 7.22-7.30 (2H, m), 7.38-7.40 (1H, m), 7.77 (1H, s), 7.85 (1H, s), 8.17 (1H, s), 9.94 (1H, s).

SU20666-0021

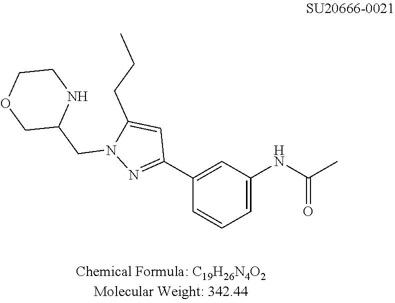 [SMILES: CCCc1cc(-c2cccc(NC(C)=O)c2)nn1CC1COCCN1]

Route for SU20666-0021

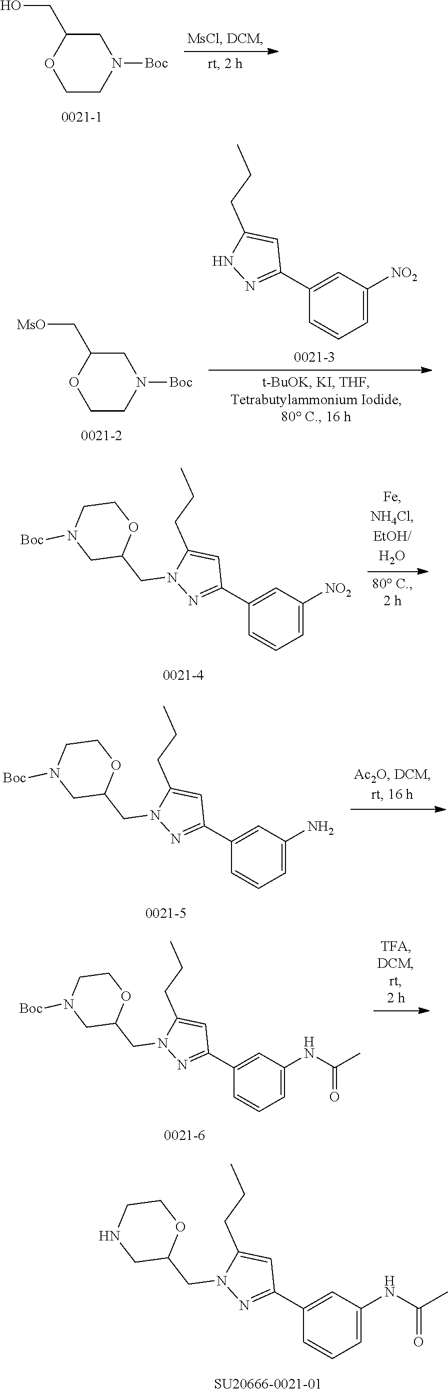

The synthesis of tert-butyl 2-(((methyl sulfonyl)oxy)methyl)morpholine-4-carboxylate (0021-2).

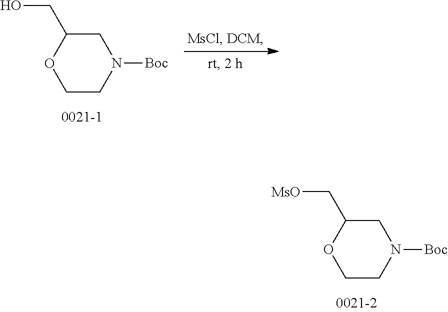 [SMILES: CC(C)(C)OC(=O)N1CCOC(CO)C1 ; CC(C)(C)OC(=O)N1CCOC(COS(C)(=O)=O)C1]

To a stirred solution of 0021-1 (700 mg, 3.2 mmol) in DCM (20 ml) was added DIEA (1.2 g, 9.7 mmol) and MsCl (443 mg, 3.9 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo to give the desired product 0021-2 (500 mg, yield: 53%) as yellow oil.

The synthesis of tert-butyl 2-((3-(3-nitrophenyl)-5-propyl-1H-pyrazol-1-yl)methyl)morpholine-4-carboxylate (0021-4).

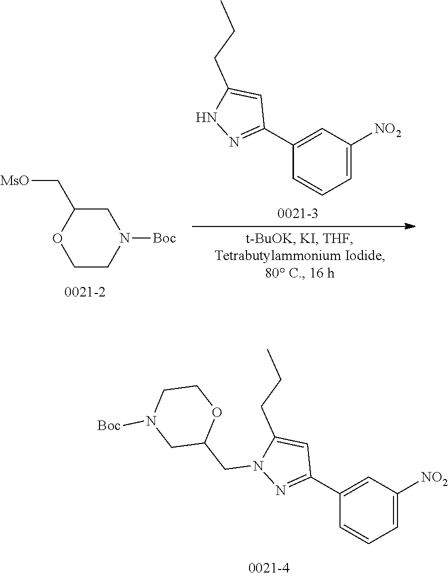 [SMILES: CC(C)(C)OC(=O)N1CCOC(COS(C)(=O)=O)C1 ; CCCc1cc(-c2cccc([N+](=O)[O-])c2)n[nH]1 ; CCCc1cc(-c2cccc([N+](=O)[O-])c2)nn1CC1CN(C(=O)OC(C)(C)C)CCO1]

To a stirred solution of 0021-2 (500 mg, 1.7 mmol) in THF (20 ml) was added KI (188 mg, 1.1 mmol), t-BuOK (190 mg, 1.7 mmol), TBAI (417 mg, 1.1 mmol) and 0021-3 (261 mg, 1.1 mmol) at rt. The resulting reaction mixture was stirred for 16 h at 80° C. Then added water, the aqueous phase was extracted with dichloromethane, the combined organic phases were dried over anhydrous sodium sulfate, filtered, and concentrated in vacuo and purified by prep-HPLC to give the desired product 0021-4 (120 mg, yield: 25%) as yellow oil.

The synthesis of tert-butyl 2-((3-(3-aminophenyl)-5-propyl-1H-pyrazol-1-yl)methyl)morpholine-4-carboxylate (0021-5).

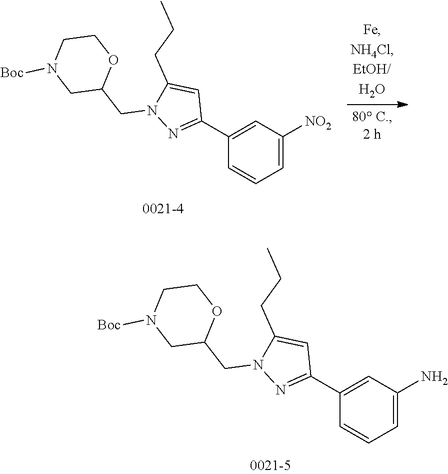 [SMILES: CCCc1cc(-c2cccc([N+](=O)[O-])c2)nn1CC1CN(C(=O)OC(C)(C)C)CCO1 ; CCCc1cc(-c2cccc(N)c2)nn1CC1CN(C(=O)OC(C)(C)C)CCO1]

To a stirred solution of 0021-4 (120 mg, 0.28 mmol) in EtOH/H₂O (6 mL/1 mL) was added Fe powder (47 mg, 0.84 mmol) and NH₄Cl (30 mg, 0.56 mmol) at rt. The resulting reaction mixture was stirred for 2 h at 80° C. Then added water, the aqueous phase was extracted with ethyl acetate, the combined organic phases were dried over anhydrous sodium sulfate, filtered, and concentrated in vacuo and purified by prep-TLC to give the desired product 0021-5 (80 mg, yield: 72%) as yellow oil.

The synthesis of tert-butyl 2-((3-(3-acetamidophenyl)-5-propyl-1H-pyrazol-1-yl)methyl)morpholine-4-carboxylate (0021-6).

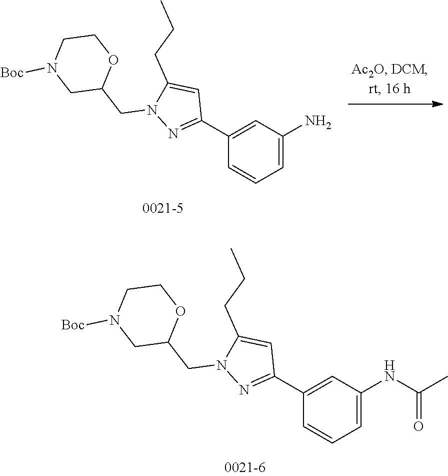 [SMILES: CCCc1cc(-c2cccc(N)c2)nn1CC1CN(C(=O)OC(C)(C)C)CCO1 ; CCCc1cc(-c2cccc(NC(C)=O)c2)nn1CC1CN(C(=O)OC(C)(C)C)CCO1]

To a stirred solution of 0021-5 (80 mg, 0.20 mmol) in DCM (10 mL) was added Ac₂O (60 mg, 0.60 mmol) at rt. The resulting reaction mixture was stirred for 16 h at rt. Then added water, the aqueous phase was extracted with DCM, the combined organic phases were dried over anhydrous sodium sulfate, filtered, and concentrated in vacuo and purified by prep-TLC to give the desired product 0021-6 (50 mg, yield: 57%) as yellow oil.

The synthesis of N-(3-(1-(morpholin-2-ylmethyl)-5-propyl-1H-pyrazol-3-yl)phenyl)acetamide (SU20666-0021).

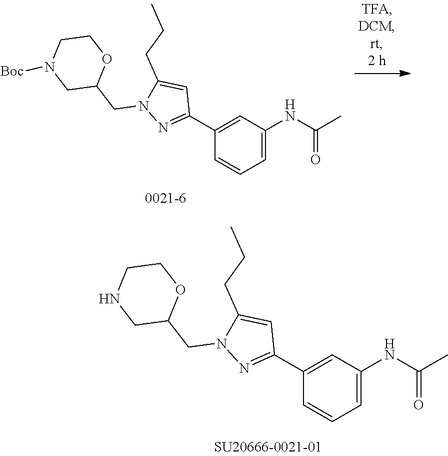 [SMILES: CCCc1cc(-c2cccc(NC(C)=O)c2)nn1CC1CN(C(=O)OC(C)(C)C)CCO1 ; CCCc1cc(-c2cccc(NC(C)=O)c2)nn1CC1CNCCO1]

To a stirred solution of compound 0021-6 (50 mg, 0.11 mmol) in DCM (10 mL) was added TFA (1 mL) at rt. The resulting reaction mixture was further stirred for 2 h at rt, then concentrated in vacuo and purified by prep-HPLC to give the desired product SU20666-0021 (18 mg, yield: 46%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.75%, Rt=1.544 min; MS Calcd.: 342.2; MS Found: 343.4 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.564 min.

¹H NMR (400 MHz, DMSO-d₆) δ 0.98 (3H, t, J=7.2 Hz), 1.63-1.68 (2H, m), 2.04 (3H, s), 2.62 (2H, t, J=7.6 Hz), 2.68-2.71 (1H, m), 2.77-2.80 (1H, m), 3.06-3.08 (1H, m), 3.15-3.20 (1H, m), 3.36-3.40 (1H, m), 3.61-3.64 (2H, m), 3.96 (2H, d, J=6.4 Hz), 6.41 (1H, s), 7.28 (1H, t, J=8.0 Hz), 7.38 (1H, d, J=7.6 Hz), 7.56 (1H, d, J=8.0 Hz), 7.95 (1H, s), 9.97 (1H, s).

SU20666-0022

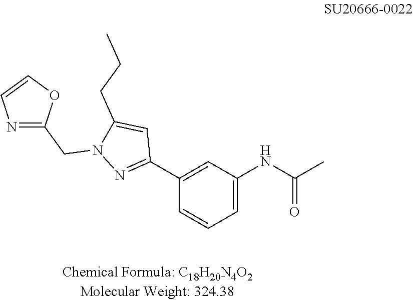 [SMILES: CCCc1cc(-c2cccc(NC(C)=O)c2)nn1Cc1ncco1]

Route for SU20666-0022

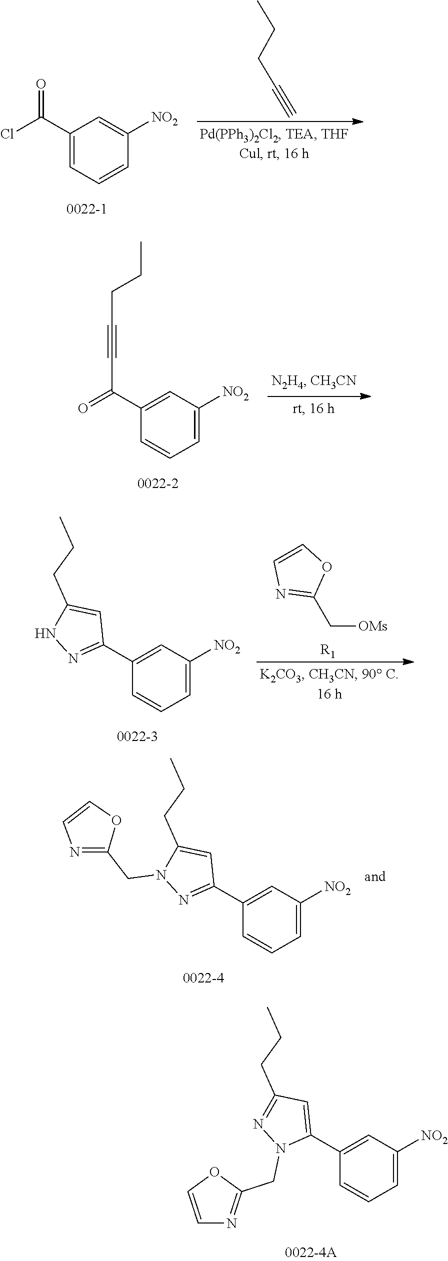

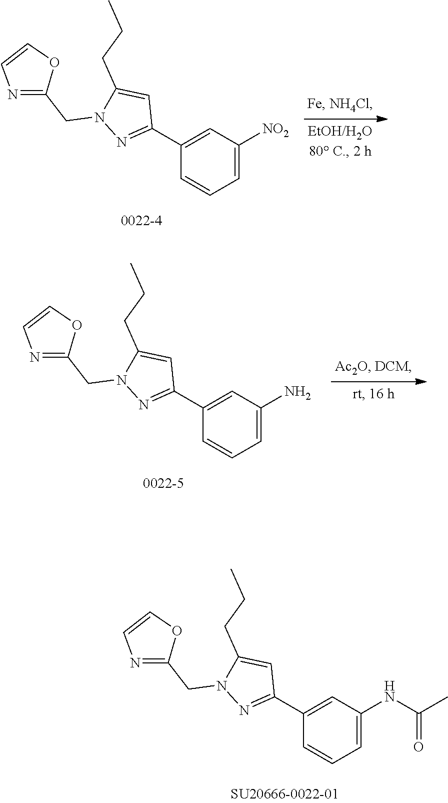 [SMILES: CCCc1cc(-c2cccc([N+](=O)[O-])c2)nn1Cc1ncco1 ; CCCc1cc(-c2cccc(N)c2)nn1Cc1ncco1 ; CCCc1cc(-c2cccc(NC(C)=O)c2)nn1Cc1ncco1]

The synthesis of 1-(3-nitrophenyl)hex-2-yn-1-one (0022-2).

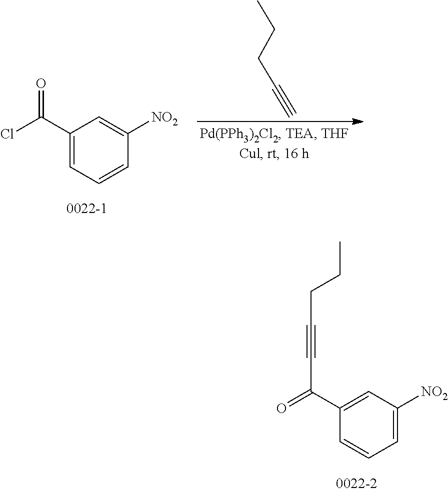

To a stirred solution of 0022-1 (9.2 g, 50.0 mmol) in THF (150 ml) was added TEA (10.0 g, 100.0 mmol), pent-1-yne (3.4 g, 50.0 mmol), Pd(PPh₃)₂Cl₂ (0.90 g) and CuI (0.50 g). The resulting reaction mixture was stirred for 16 h at rt under argon atomosphere. Then concentrated to remove the solvent and added water, the aqueous phase was extracted with dichloromethane, the combined organic phases were dried over anhydrous sodium sulfate, filtered, and concentrated in vacuo, the crude was purified by C.C. to give the desired product 0022-2 (6.8 g, yield: 63%) as yellow oil.

The synthesis of 3-(3-nitrophenyl)-5-propyl-1H-pyrazole (0022-3).

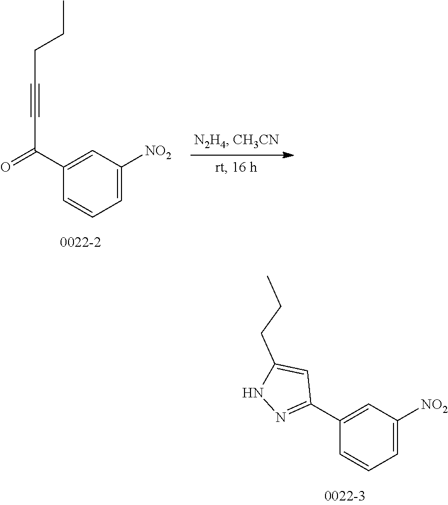 [SMILES: CCCC#CC(=O)c1cccc([N+](=O)[O-])c1 ; CCCc1cc(-c2cccc([N+](=O)[O-])c2)n[nH]1]

To a stirred solution of 0022-2 (2.0 g, 1.7 mmol) in acetonitrile (20 ml) was added N₂H₄ (98%, 1.3 g, 27.6 mmol) at rt. The resulting reaction mixture was stirred for 16 h at rt. Then added water, the aqueous phase was extracted with ethyl acetate, the combined organic phases were dried over anhydrous sodium sulfate, filtered, and concentrated in vacuo and purified by C.C. to give the desired product 0022-3 (2.1 g, yield: 99%) as a yellow solid.

The synthesis of 2-((3-(3-nitrophenyl)-5-propyl-1H-pyrazol-1-yl)methyl)oxazole (0022-4) and 2-((5-(3-nitrophenyl)-3-propyl-1H-pyrazol-1-yl)methyl)oxazole (0022-4A)

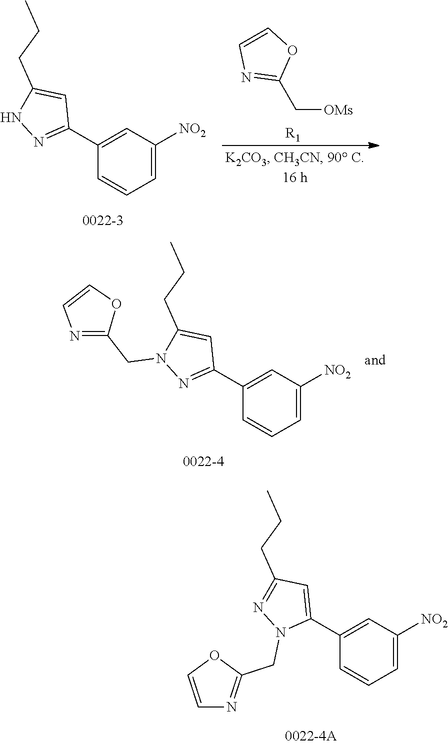

To a stirred solution of 0022-3 (500 mg, 2.1 mmol) in acetonitrile (20 mL) was added oxazol-2-ylmethyl methanesulfonate (R₁, 450 mg, 2.6 mmol) and K₂CO₃ (360 mg, 2.6 mmol) at rt. The resulting reaction mixture was stirred for 16 h at 90° C. Then added water, the aqueous phase was extracted with ethyl acetate, the combined organic phases were dried over anhydrous sodium sulfate, filtered, and concentrated in vacuo and purified by prep-HPLC to give the desired product 0022-4 (200 mg, yield: 30%) as yellow solid and product 0022-4A (30 mg, yield: 4.5%) as yellow solid.

The synthesis of 3-(1-(oxazol-2-ylmethyl)-5-propyl-1H-pyrazol-3-yl)aniline (0022-5).

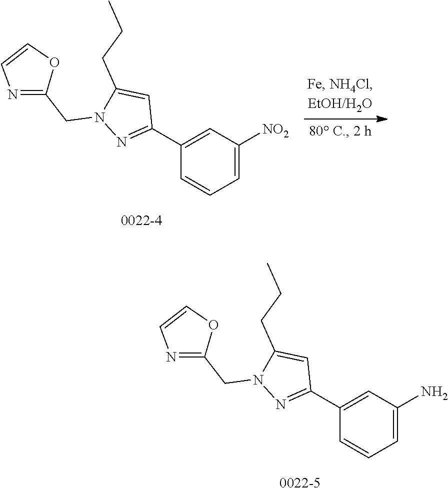 [SMILES: CCCc1cc(-c2cccc([N+](=O)[O-])c2)nn1Cc1ncco1 ; CCCc1cc(-c2cccc(N)c2)nn1Cc1ncco1]

To a stirred solution of 0022-4 (200 mg, 0.64 mmol) in EtOH/H₂O (10 mL/2 mL) was added Fe powder (180 mg, 3.2 mmol) and NH₄Cl (170 mg, 3.2 mmol) at rt. The resulting reaction mixture was stirred for 2 h at 80° C. Then added water, the aqueous phase was extracted with ethyl acetate, the combined organic phases were dried over anhydrous sodium sulfate, filtered, and concentrated in vacuo to give the desired product 0022-5 (160 mg, yield: 89%) as yellow oil.

The synthesis of N-(3-(1-(oxazol-2-ylmethyl)-5-propyl-1H-pyrazol-3-yl)phenyl)acetamide (SU20666-0022).

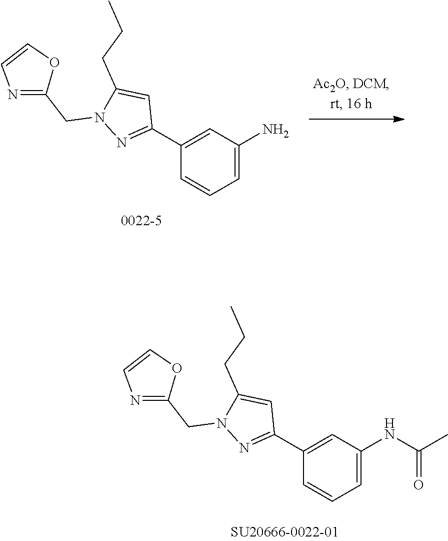 [SMILES: CCCc1cc(-c2cccc(N)c2)nn1Cc1ncco1 ; CCCc1cc(-c2cccc(NC(C)=O)c2)nn1Cc1ncco1]

To a stirred solution of 0022-5 (100 mg, 0.35 mmol) in DCM (10 mL) was added Ac₂O (72 mg, 0.70 mmol) at rt. The resulting reaction mixture was stirred for 16 h at rt. Then added water, the aqueous phase was extracted with DCM, the combined organic phases were dried over anhydrous sodium sulfate, filtered, and concentrated in vacuo and purified by prep-HPLC to give the desired product SU20666-0022 (72 mg, yield: 63%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.24%, Rt=1.659 min; MS Calcd.: 324.1; MS Found: 325.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.218 min.

¹H NMR (400 MHz, DMSO-d₆) δ 0.96 (3H, t, J=7.2 Hz), 1.61-1.67 (2H, m), 2.03 (3H, s), 2.67 (2H, t, J=7.6 Hz), 5.51 (2H, s), 6.49 (1H, s), 7.22 (1H, s), 7.28 (1H, t, J=8.0 Hz), 7.38 (1H, d, J=8.0 Hz), 7.56 (1H, d, J=8.8 Hz), 7.95 (1H, s), 8.10 (1H, s), 9.96 (1H, s).

SU20666-0026

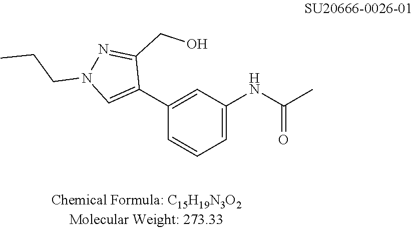 [SMILES: CCCn1cc(-c2cccc(NC(C)=O)c2)c(CO)n1]

Route for SU20666-0026

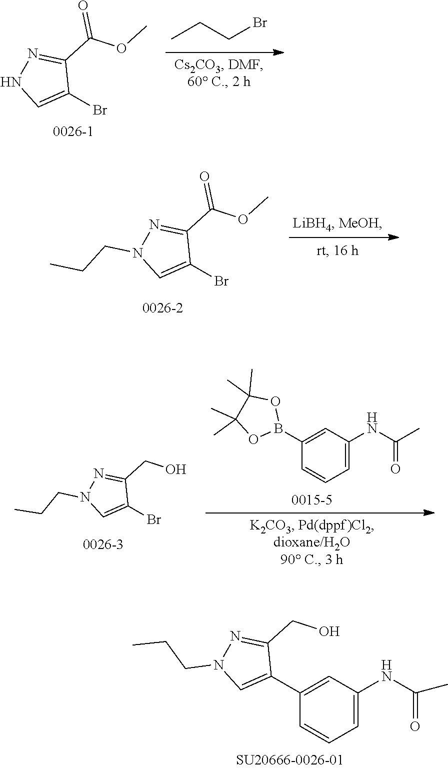

The synthesis of methyl 4-bromo-1-propyl-1H-pyrazole-3-carboxylate (0026-2).

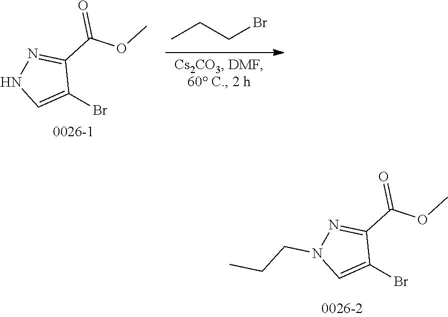

To a stirred solution of 0026-1 (9.9 g, 48.4 mmol) in DMF (300 ml) was added 1-bromopropane (6.2 g, 50.8 mmol), Cs₂CO₃ (23.0 g, 70.5 mmol). The resulting reaction mixture was heated to 60° C. for 2 h. Then added water, the aqueous phase was extracted with dichloromethane, the combined organic phases were dried over anhydrous sodium sulfate, filtered, and concentrated in vacuopurified by C.C. to give the desired product 0026-2 (6,1 g, yield: 51%) as colorless oil.

The synthesis of (4-bromo-1-propyl-1H-pyrazol-3-yl)methanol (0026-3).

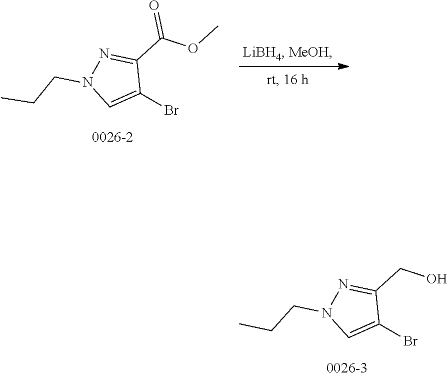

To a stirred solution of 0026-2 (1.2 g, 4.88 mmol) in methanol (20 mL) was added LiBH₄ (153 mg, 7.3 mmol) at rt. The resulting reaction mixture was stirred for 16 h at rt. Then added water, the aqueous phase was extracted with EA, the combined organic phases were dried over anhydrous sodium sulfate, filtered, and concentrated in vacuo and purified by C.C. to give the desired product 0026-3 (1.0 g, yield: 94%) as colorless oil.

The synthesis of N-(3-(3-(hydroxymethyl)-1-propyl-1H-pyrazol-4-yl)phenyl)acetamide (SU20666-0026-01).

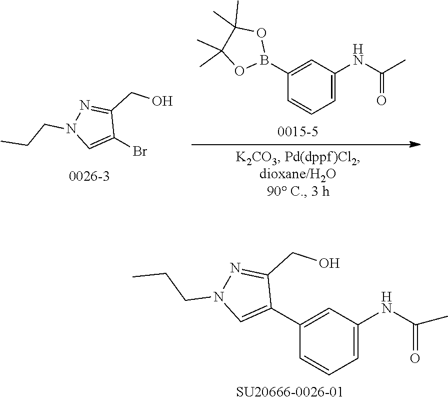 [SMILES: CCCn1cc(Br)c(CO)n1 ; CC(=O)Nc1cccc(B2OC(C)(C)C(C)(C)O2)c1 ; CCCn1cc(-c2cccc(NC(C)=O)c2)c(CO)n1]

To a solution of compound 0026-3 (250 mg, 1.2 mmol) in dioxane/water (6 mL/2 mL) was added 015-5 (328 mg, 1.3 mmol), K₂CO₃ (476 mg, 3.4 mmol), Pd(dppf)Cl₂(90 mg). The resulting reaction mixture was heated to 90° C. and stirred for 3 h, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product SU20666-0026 (40 mg, yield: 13%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.28%, Rt=1.377 min; MS Calcd.: 273.1; MS Found: 274.7 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.34%, Rt=6.576 min.

¹H NMR (400 MHz, DMSO-d₆) δ 0.86 (3H, t, J=7.6 Hz), 1.78-1.84 (2H, m), 2.04 (3H, s), 4.04 (2H, t, J=6.8 Hz), 4.47 (2H, d, J=5.2 Hz), 5.04 (1H, t, J=5.2 Hz), 7.27-7.29 (2H, m), 7.45-7.47 (2H, m), 7.69 (1H, s), 7.90 (1H, s), 9.91(1H, s).

SU20666-0027 and SU20666-0029

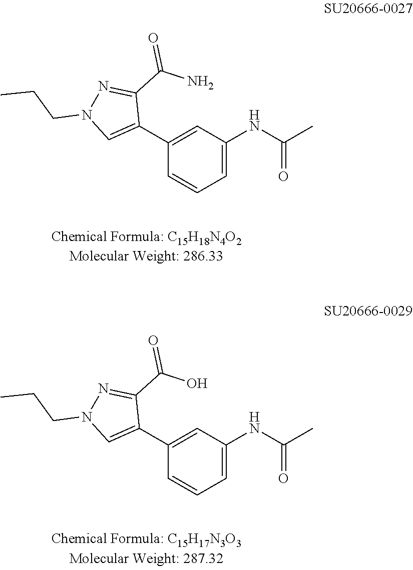

Route for SU20666-0027 and SU20666-0029

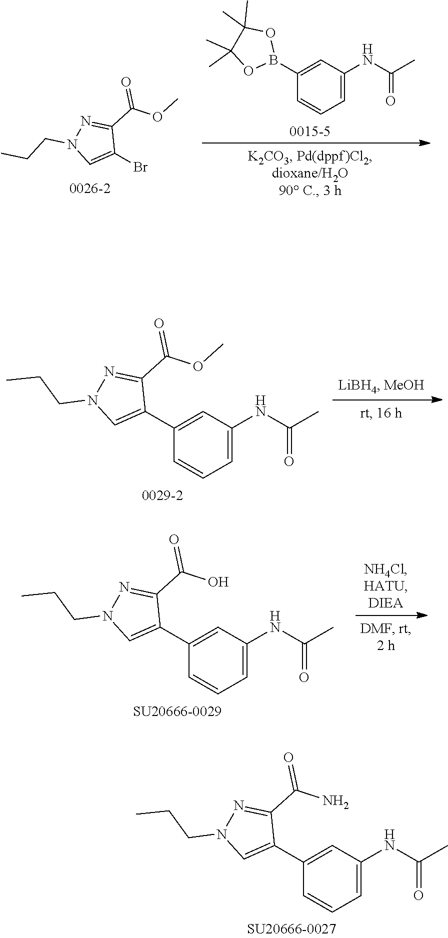

The synthesis of methyl 4-(3-acetamidophenyl)-1-propyl-1H-pyrazole-3-carboxylate (0029-2).

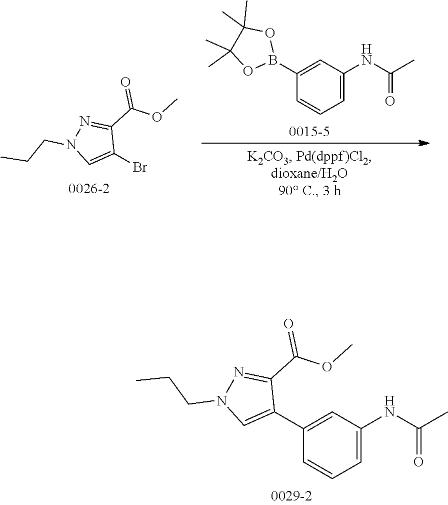 [SMILES: CCCn1cc(Br)c(C(=O)OC)n1 ; CC(=O)Nc1cccc(B2OC(C)(C)C(C)(C)O2)c1 ; CCCn1cc(-c2cccc(NC(C)=O)c2)c(C(=O)OC)n1]

To a solution of compound 0026-2 (300 mg, 1.2 mmol) in dioxane/water (50 mL/5 mL) was added 015-5 (317 mg, 1.2 mmol), K₂CO₃ (200 mg, 1.5 mmol), Pd(dppf)Cl₂ (30 mg). The resulting reaction mixture was heated to 90° C. and stirred for 3 h, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product 0029-2 (200 mg, yield: 54%) as a yellow solid.

The synthesis of 4-(3-acetamidophenyl)-1-propyl-1H-pyrazole-3-carboxylic acid (SU20666-0029).

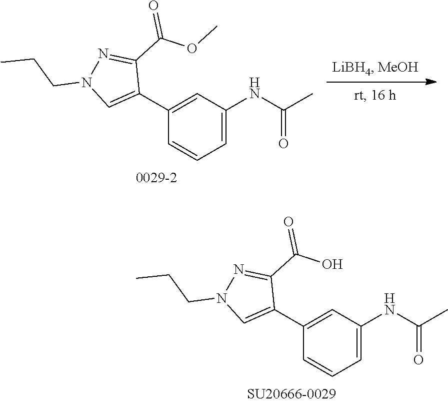

To a stirred solution of 0029-2 (60 mg, 0.20 mmol) in methanol (10 mL) was added LiOH (42 mg, 1.0 mmol) at rt. The resulting reaction mixture was stirred for 16 h at rt. Then added water, the aqueous phase was extracted with EA, the combined organic phases were dried over anhydrous sodium sulfate, filtered, and concentrated in vacuo and purified by prep-HPLC to give the desired product SU20666-0029 (20 mg, yield: 35%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.369 min; MS Calcd.: 287.1; MS Found: 288.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 93.68%, Rt=6.387 min.

¹H NMR (400 MHz, DMSO-d₆) δ 0.86 (3H, t, J=7.2 Hz), 1.81-1.87 (2H, m), 2.03 (3H, s), 4.13 (2H, t, J=6.8 Hz), 7.09 (1H, d, J=7.6 Hz), 7.25 (1H, t, J=7.6 Hz), 7.53 (1H, d, J=8.4 Hz), 7.61 (1H, s), 7.95 (1H, s), 9.94(1H, s).

The synthesis of 4-(3-acetamidophenyl)-1-propyl-1H-pyrazole-3-carboxamide (SU20666-0027).

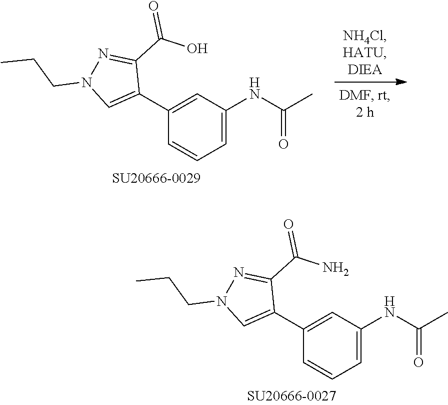

To a solution of compound SU20666-0029 (60 mg, 0.21 mmol) in DMF (10 mL) was added NH₄Cl (22 mg, 0.42 mmol), DIEA (134 mg, 1.0 mmol) and HATU (160 mg, 0.42 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0029 (25 mg, yield: 42%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.70%, Rt=1.392 min; MS Calcd.: 286.1; MS Found: 287.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.80%, Rt=6.718 min.

¹H NMR (400 MHz, DMSO-d₆) δ 0.88 (3H, t, J=7.2 Hz), 1.82-1.88 (2H, m), 2.03 (3H, s), 4.10 (2H, t, J=6.8 Hz), 7.17-7.25 (3H, m), 7.41 (1H, s), 7.52 (1H, d, J=8.4 Hz), 7.63 (1H, s), 7.94 (1H, s), 9.92 (1H, s).

SU20666-0033

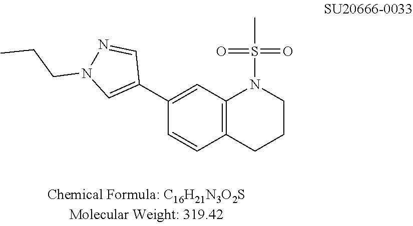 [SMILES: CCCn1cc(-c2ccc3c(c2)N(S(C)(=O)=O)CCC3)cn1]

Route for SU20666-0033

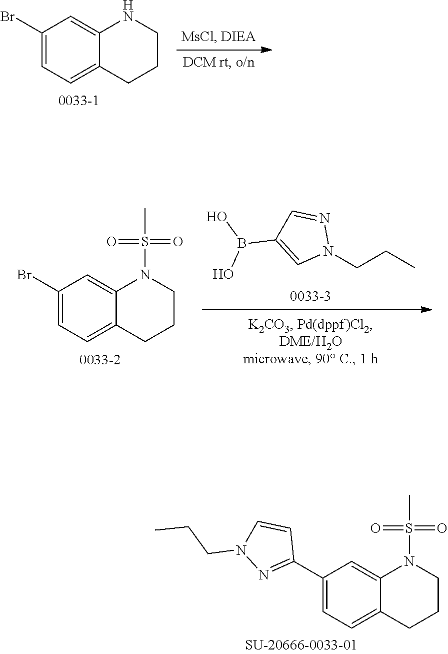 [SMILES: Brc1ccc2c(c1)NCCC2 ; CS(=O)(=O)N1CCCc2ccc(Br)cc21 ; CCCn1cc(B(O)O)cn1 ; CCCn1ccc(-c2ccc3c(c2)N(S(C)(=O)=O)CCC3)n1]

The synthesis of 7-bromo-1-(methylsulfonyl)-1,2,3,4-tetrahydroquinoline (0033-2).

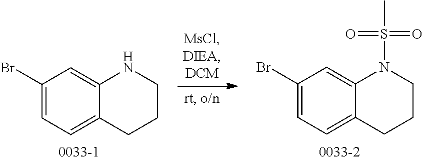

To a solution of compound 0033-2 (1.0 g, 4.7 mmol) in DCM (15 mL) was added DIEA (1.8 g, 14.1 mmol) and MsCl (0.65 g, 5.7 mmol) at 0° C. The resulting reaction mixture was stirred for 16 h at rt, then added water, the aqueous phase was extracted with EA, the combined organic phases were dried over anhydrous sodium sulfate, filtered, and concentrated, the crude was purified by C.C. to give the desired product 0033-2 (700 mg, yield: 52%) as a yellow solid.

The synthesis of 4-(3-acetamidophenyl)-1-propyl-1H-pyrazole-3-carboxylic acid (SU20666-0033).

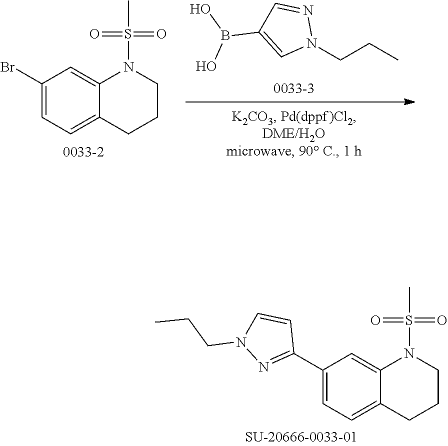 [SMILES: CS(=O)(=O)N1CCCc2ccc(Br)cc21 ; CCCn1cc(B(O)O)cn1 ; CCCn1ccc(-c2ccc3c(c2)N(S(C)(=O)=O)CCC3)n1]

To a solution of compound 0033-2 (200 mg, 0.70 mmol) in DME/water (5 mL/1 mL) was added 0033-3 (128 mg, 0.83 mmol), K₂CO₃ (193 mg, 1.4 mmol) and Pd(dppf)Cl₂ (20 mg). The resulting reaction mixture was heated to 90° C. and stirred for 0.5 h at MW conditions, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product SU20666-0033 (18 mg, yield: 8%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.44%, Rt=1.761 min; MS Calcd.: 319.1; MS Found: 320.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.338 min.

¹H NMR (400 MHz, DMSO-d₆) δ 0.84 (3H, t, J=7.2 Hz), 1.78-1.83 (2H, m), 1.91-1.94 (2H, m), 2.81 (2H, t, J=6.4 Hz), 3.00 (3H, s), 3.67-3.70 (2H, m), 4.05 (2H, t, J=7.2 Hz), 7.34-7.38 (2H, m), 7.51 (1H, d, J=8.4 Hz), 7.82 (1H, s), 8.12 (1H, s).

SU20666-0034

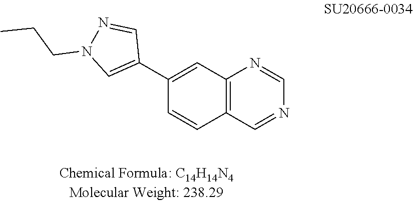 [SMILES: CCCn1cc(-c2ccc3cncnc3c2)cn1]

Route for SU20666-0034

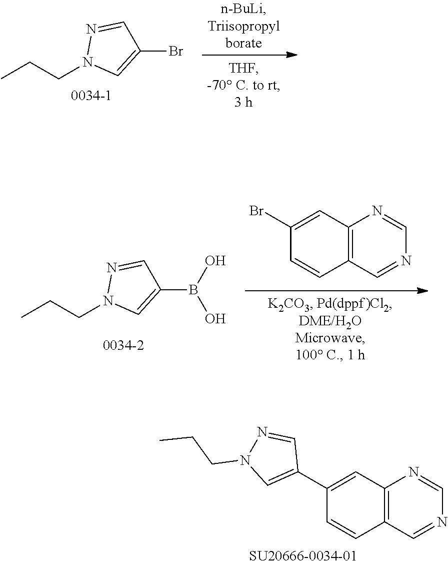

The synthesis of (1-propyl-1H-pyrazol-4-yl)boronic acid (0034-2).

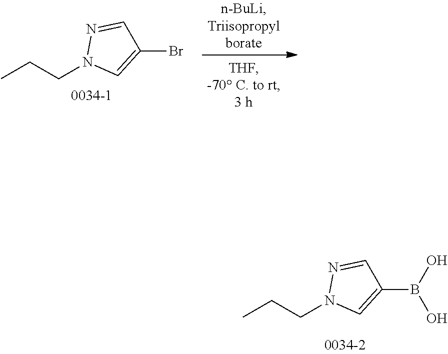

To a solution of compound 0034-2 (2.5 g, 13.2 mmol) in THF (30 mL) was added n-BuLi (2.5 M, 6.3 mL, 15.9 mmol) at −78° C. The resulting reaction mixture was stirred for 1 h at −78° C., then added triisopropyl borate (5.0 g, 16.4 mmol) slowly, then the reaction mixture was stirred for 3 h at rt. Water was added the aqueous phase was extracted with EA, the combined organic phases were dried over anhydrous sodium sulfate, filtered, and concentrated, the crude was purified by C.C. to give the desired product 0034-2 (400 mg, yield: 30%) as a yellow solid.

The synthesis of 7-(1-propyl-1H-pyrazol-4-yl)quinazoline (SU20666-0034).

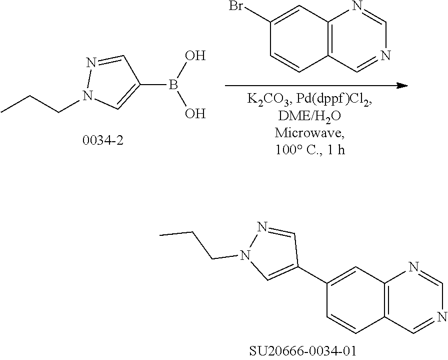 [SMILES: CCCn1cc(B(O)O)cn1 ; Brc1ccc2cncnc2c1 ; CCCn1cc(-c2ccc3cncnc3c2)cn1]

To a solution of compound 0034-2 (266 mg, 0.86 mmol) in DME/water (5 mL/1 mL) was added 7-bromoquinazoline (300 mg, 0.72 mmol), K₂CO₃ (400 mg, 1.4 mmol) and Pd(dppf)Cl₂ (20 mg). The resulting reaction mixture was heated to 100° C. and stirred for 1 h at MW conditions, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product SU20666-0034 (12 mg, yield: 7%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.89%, Rt=1.475 min; MS Calcd.: 238.1; MS Found: 239.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.344 min.

¹H NMR (400 MHz, CDCl₃) δ 0.99 (3H, t, J=7.2 Hz), 1.95-2.00 (2H, m), 4.17 (2H, t, J=7.2 Hz), 7.80-7.85 (2H, m), 7.91-7.93 (1H, m), 7.97 (1H, s), 8.10 (1H, s), 9.29 (1H, s), 9.32 (1H, s).

SU20666-0035

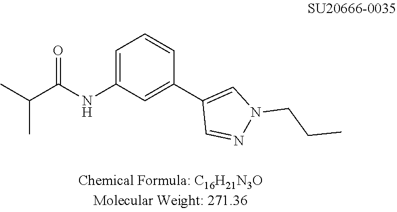 [SMILES: CCCn1cc(-c2cccc(NC(=O)C(C)C)c2)cn1]

Route for SU20666-0035

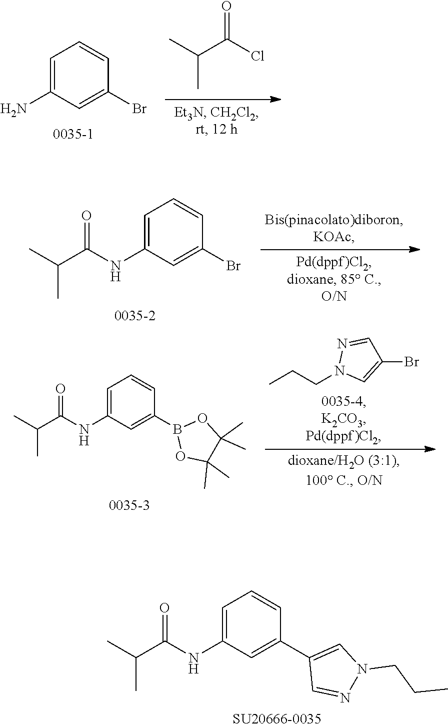

The synthesis of N-(3-bromophenyl)isobutyramide (0035-2).

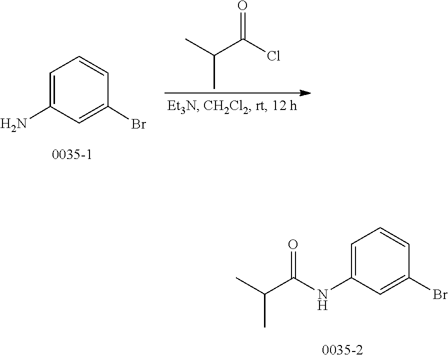

To a solution of compound 0035-2 (1.0 g, 5.8 mmol) in DCM (10 mL) was added TEA (648 mg, 6.4 mmol) and isobutyryl chloride (650 mg, 6.1 mmol) at 0° C. The resulting reaction mixture was stirred for 12 h at rt. Water was added the aqueous phase was extracted with DCM, the combined organic phases were dried over anhydrous sodium sulfate, filtered, and concentrated, the crude was purified by C.C. to give the desired product 0035-2 (1.2 g, yield: 85%) as a white solid.

The synthesis of N-(3-(4,4,5,5-tetramethyl-1,3,2-dioxaborolan-2-yl)phenyl)isobutyramide (0035-3).

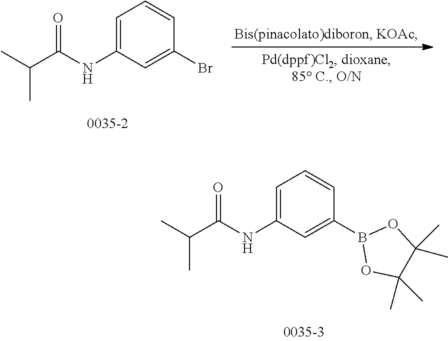 [SMILES: CC(C)C(=O)Nc1cccc(Br)c1 ; CC(C)C(=O)Nc1cccc(B2OC(C)(C)C(C)(C)O2)c1]

To a stirred solution of compound 0035-2 (240 mg, 1.0 mmol) in dioxane (5 mL) was added bis(pinacolato)diboron (381 mg, 1.5 mmol), KOAc (294 mg, 2.0 mmol), Pd(dppf)Cl₂ (20 mg). The resulting reaction mixture was heated to 85° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 0035-3 (230 mg, yield: 80%) as a white solid.

The synthesis of N-(3-(1-propyl-1H-pyrazol-4-yl)phenyl)isobutyramide (SU20666-0035).

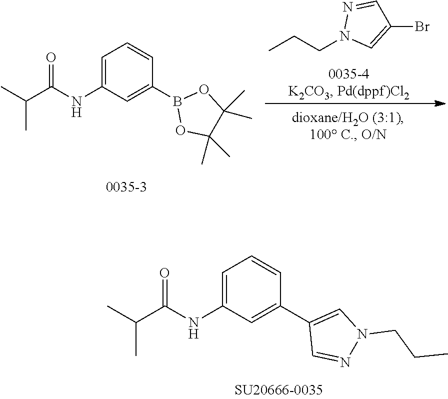

To a solution of compound 0035-3 (153 mg, 0.53 mmol) in dioxane/water (3 mL/1 mL) was added 0035-4 (100 mg, 0.53 mmol), K₂CO₃ (146 mg, 1.1 mmol) and Pd(dppf)Cl₂ (20 mg). The resulting reaction mixture was heated to 100° C. and stirred for 16 h, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product SU20666-0035 (70 mg, yield: 49%) as a pale yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 97.15%, Rt=1.684 min; MS Calcd.: 271.2; MS Found: 272.3 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 97.60%, Rt=8.419 min.

¹H NMR (400 MHz, DMSO-d₆) δ 0.85 (3H, t, J=7.2 Hz), 1.11 (6H, d, J=6.8 Hz), 1.79-1.84 (2H, m), 2.59-2.62 (1H, m), 4.08 (2H, t, J=6.8 Hz), 7.21-7.28 (2H, m), 7.40 (1H, dt, J=7.6, 1.6 Hz), 7.77 (1H, s), 7.83 (1H, s), 8.09 (1H, s), 9.82 (1H, s).

SU20666-0036

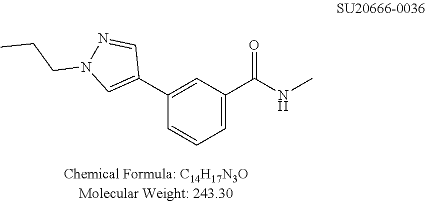 [SMILES: CCCn1cc(-c2cccc(C(=O)NC)c2)cn1]

Route for SU20666-0036

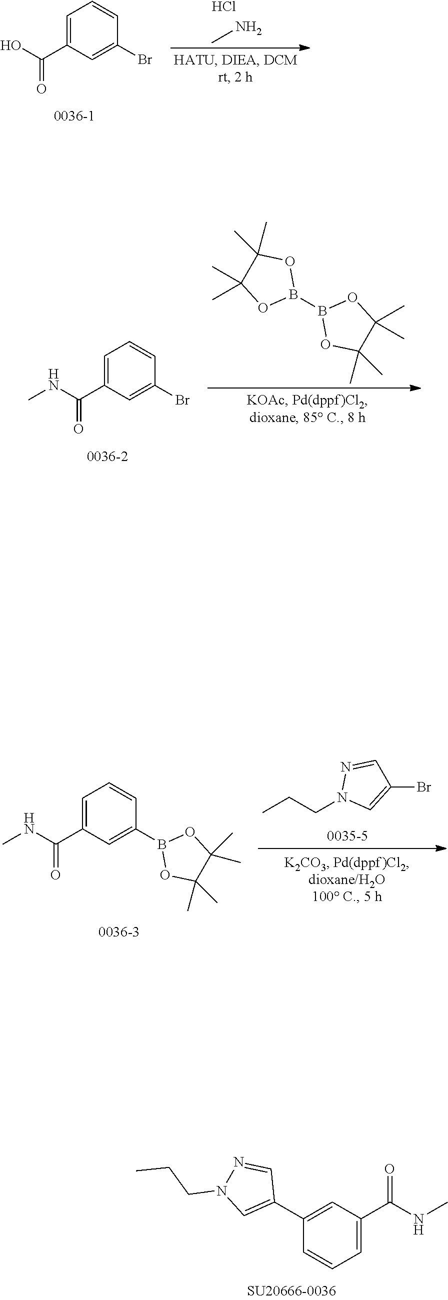

The synthesis of 3-bromo-N-methylbenzamide (0036-2).

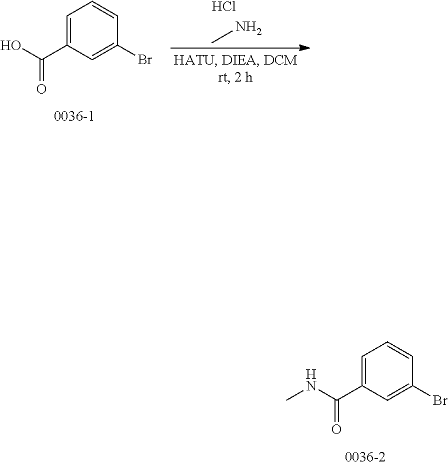

To a solution of compound 0036-1 (4.0 g, 19.9 mmol) in DCM (10 mL) was added DIEA (12.8 g, 99.5 mmol), HATU (11.3 g, 29.8 mmol) and methanamine hydrochloride (2.7 g, 39.8 mmol) at 0° C. The resulting reaction mixture was stirred for 2 h at rt. Water was added the aqueous phase was extracted with DCM, the combined organic phases were dried over anhydrous sodium sulfate, filtered, and concentrated, the crude was purified by C.C. to give the desired product 0036-2 (4.0 g, yield: 94%) as colorless oil.

The synthesis of N-methyl-3-(4,4,5,5-tetramethyl-1,3,2-dioxaborolan-2-yl)benzamide (0036-3).

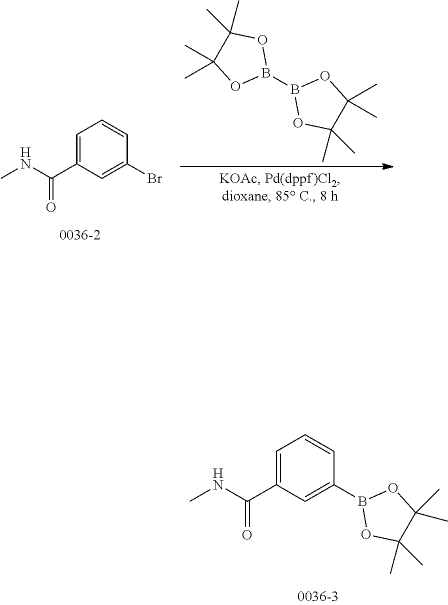

To a stirred solution of compound 0036-2 (2.0 g, 9.3 mmol) in dioxane (50 mL) was added bis(pinacolato)diboron (4.7 g, 18.6 mmol), KOAc (1.8 g, 18.6 mmol), Pd(dppf)Cl₂ (200 mg). The resulting reaction mixture was heated to 85° C. and stirred for 8 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 0036-3 (1.4 g, yield: 57%) as a yellow solid.

The synthesis of N-methyl-3-(1-propyl-1H-pyrazol-4-yl)benzamide (SU20666-0036).

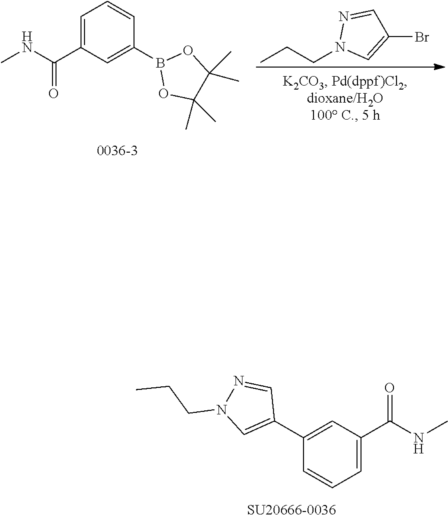

To a solution of compound 0036-3 (330 mg, 1.27 mmol) in dioxane/water (20 mL/2 mL) was added 4-bromo-1-propyl-1H-pyrazole (200 mg, 1.1 mmol), K₂CO₃ (290 mg, 2.1 mmol) and Pd(dppf)Cl₂ (20 mg). The resulting reaction mixture was heated to 100° C. and stirred for 5 h, then concentrated in vacuo to remove the solvent and added water, the aqueous phase was extracted with dichloromethane, the combined organic phases were dried over anhydrous sodium sulfate, filtered, and concentrated, the crude was purified by prep-HPLC to give the desired product SU20666-0036 (70 mg, yield: 37%) as yellow oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.455 min; MS Calcd.: 243.1; MS Found: 244.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 95.41%, Rt=7.200 min.

¹H NMR (400 MHz, DMSO-d₆) δ 0.85 (3H, t, J=7.6 Hz), 1.79-1.85 (2H, m), 2.80 (3H, d, J=4.4 Hz), 4.09 (2H, t, J=7.2 Hz), 7.43 (1H, t, J=7.6 Hz), 7.61-7.64 (1H, m), 7.69-7.71 (1H, m), 7.91 (1H, s), 8.00-8.01 (1H, m), 8.22 (1H, s), 8.44 (1H, d, J=4.4 Hz).

SU20666-0037

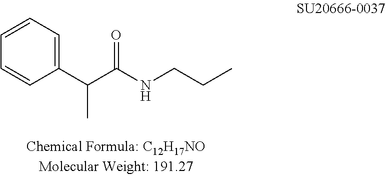

Route for SU20666-0037

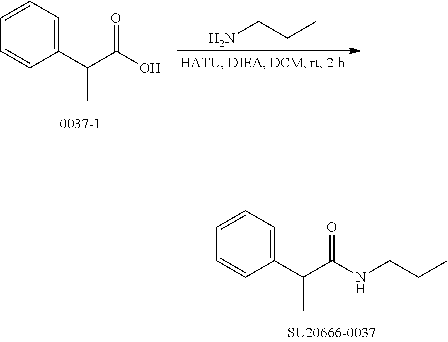

The synthesis of 2-phenyl-N-propylpropanamide (SU20666-0037).

To a solution of compound 0037-1 (200 mg, 1.3 mmol) in DCM (10 mL) was added DIEA (0.75 mL, 4.0 mmol), HATU (760 mg, 2.0 mmol) and propan-1-amine (94 mg, 1.6 mmol) at 0° C. The resulting reaction mixture was stirred for 2 h at rt. Water was added the aqueous phase was extracted with DCM, the combined organic phases were dried over anhydrous sodium sulfate, filtered, and concentrated, the crude was purified by C.C. to give the desired product SU20666-0037 (96 mg, yield: 38%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.624 min; MS Calcd.: 191.1; MS Found: 192.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.265 min.

¹H NMR (400 MHz, DMSO-d₆) δ 0.77 (3H, t, J=7.2 Hz), 1.30-1.39 (5H, m), 2.97 (2H, q, J=6.0 Hz), 3.57 (1H, q, J=6.8 Hz), 7.18-7.22 (1H, m), 7.26-7.32 (4H, m), 7.90 (1H, s).

SU20666-0038

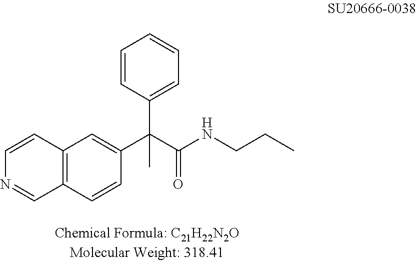 [SMILES: CCCNC(=O)C(C)(c1ccccc1)c1ccc2cnccc2c1]

Route for SU20666-0038

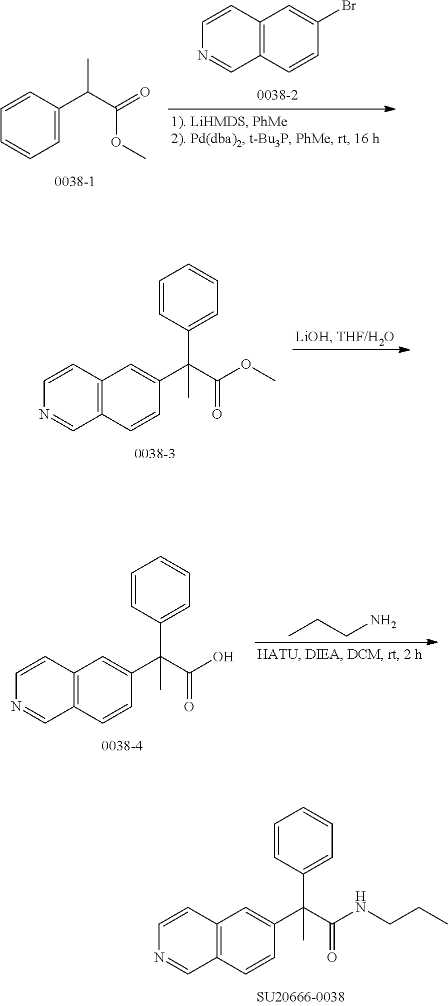

The synthesis of methyl 2-(isoquinolin-6-yl)-2-phenylpropanoate (0038-3).

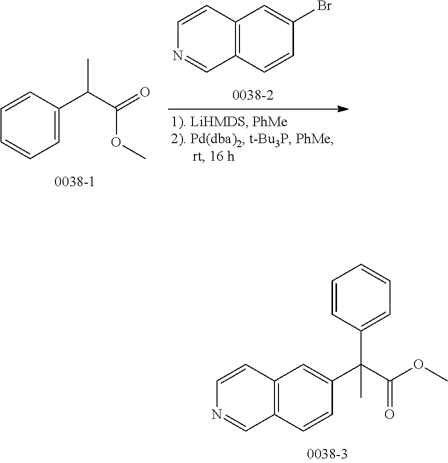 [SMILES: COC(=O)C(C)c1ccccc1 ; Brc1ccc2cnccc2c1 ; COC(=O)C(C)(c1ccccc1)c1ccc2cnccc2c1]

To a solution of compound 0038-1 (590 mg, 3.6 mmol) in toluene (10 mL) was added LDA (2.0 M, 2.1 mL, 4.3 mmol) at −78° C. and stirred at this temperature for 10 min. Pd₂(dba)₃ (50 mg) and 0038-2 (500 mg, 2.4 mmol) was added and stirred at this temperature for 10 min, then t-Bu₃P (242 mg, 1.2 mmol) in toluene (10 mL) was added and the resulting reaction mixture was stirred for 16 h at rt. Water was added the aqueous phase was extracted with EA, the combined organic phases were dried over anhydrous sodium sulfate, filtered, and concentrated, the crude was purified by C.C. to give the desired product 0038-3 (130 mg, yield: 18%) as colorless oil.

The synthesis of 2-(isoquinolin-6-yl)-2-phenylpropanoic acid (0038-4).

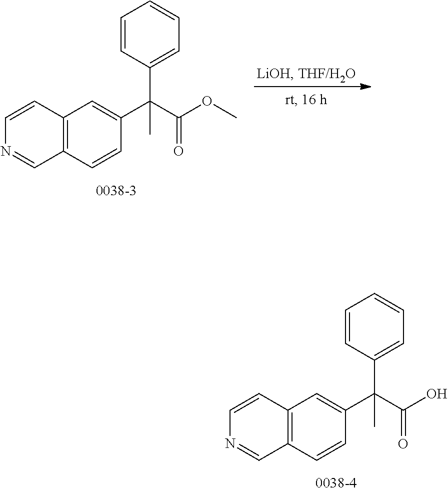 [SMILES: COC(=O)C(C)(c1ccccc1)c1ccc2cnccc2c1 ; CC(C(=O)O)(c1ccccc1)c1ccc2cnccc2c1]

To a stirred solution of 0038-3 (130 mg, 0.45 mmol) in THF/H₂O (10 mL/2 mL) was added LiOH (96 mg, 2.2 mmol) at rt. The resulting reaction mixture was stirred for 16 h at rt. Then added water, the aqueous phase was extracted with EA, the combined organic phases were dried over anhydrous sodium sulfate, filtered, and concentrated in vacuo and purified by prep-HPLC to give the desired product 0038-4 (85 mg, yield: 69%) as a white solid.

The synthesis of 2-(isoquinolin-6-yl)-2-phenyl-N-propylpropanamide (SU20666-0038).

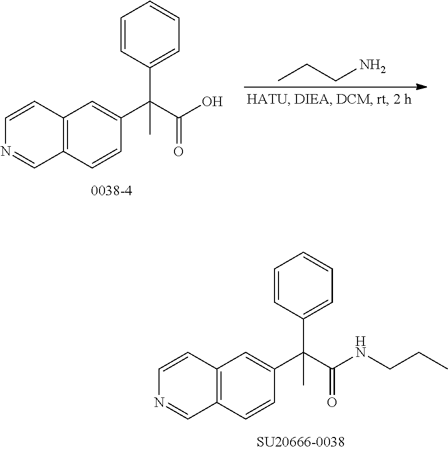

To a solution of compound 0038-4 (85 mg, 0.30 mmol) in DCM (10 mL) was added propan-1-amine (36 mg, 0.60 mmol), DIEA (190 mg, 1.5 mmol) and HATU (170 mg, 0.45 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0038 (41 mg, yield: 42%) as yellow oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 92.80%, Rt=1.948 min; MS Calcd.: 318.2; MS Found: 319.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.119 min.

¹H NMR (400 MHz, DMSO-d₆) δ 0.78 (3H, t, J=7.2 Hz), 1.40-1.45 (2H, m), 1.97 (3H, s), 3.07 (2H, q, J=6.4 Hz), 7.21-7.36 (5H, m), 7.48-7.51 (2H, m), 7.66 (1H, s), 7.74 (1H, d, J=5.6 Hz), 8.02 (1H, d, J=8.8 Hz), 8.47 (1H, d, J=5.6 Hz), 9.26 (1H, s).

SU20666-0040

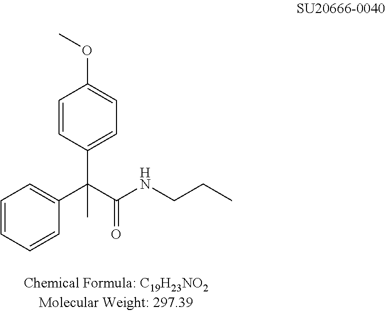 [SMILES: CCCNC(=O)C(C)(c1ccccc1)c1ccc(OC)cc1]

Route for SU20666-0040

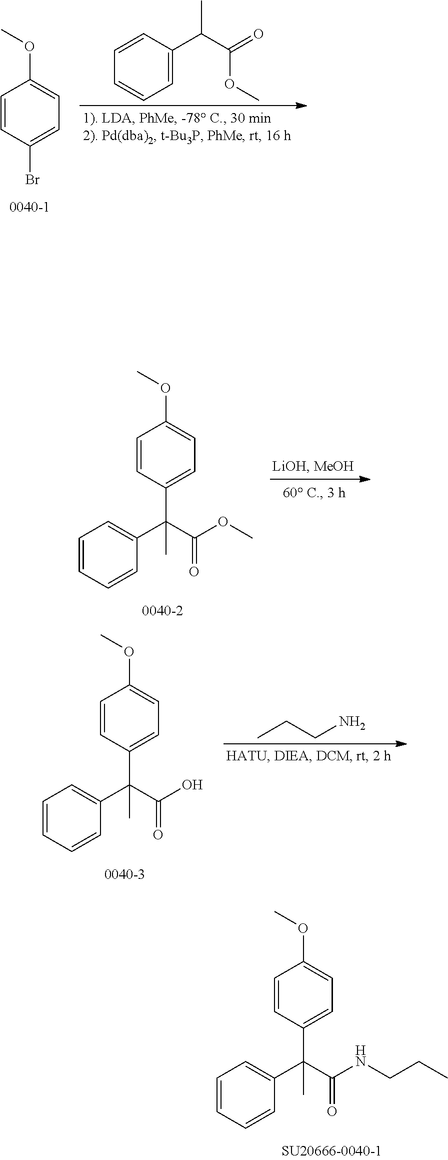

The synthesis of methyl 2-(4-methoxyphenyl)-2-phenylpropanoate (0040-2).

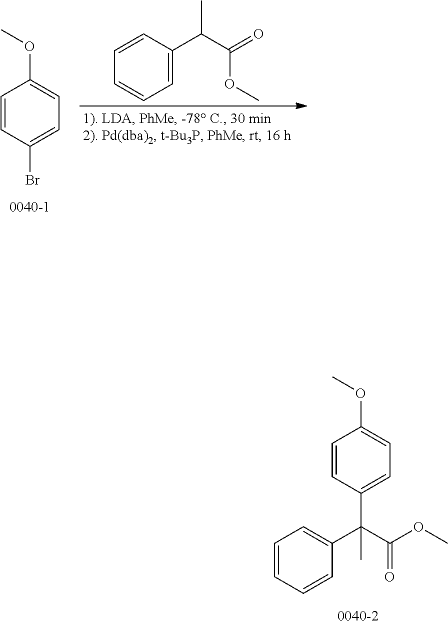

To a solution of compound 0040-1 (600 mg, 3.2 mmol) in toluene (10 mL) was added LDA (2.0 M, 2.9 mL, 5.8 mmol) at −78° C. and stirred at this temperature for 10 min. Pd₂(dba)₃ (50 mg) and 0038-2 (790 mg, 4.8 mmol) was added and stirred at this temperature for 10 min, then t-Bu₃P (323 mg, 1.6 mmol) in toluene (10 mL) was added and the resulting reaction mixture was stirred for 16 h at rt. Water was added the aqueous phase was extracted with EA, the combined organic phases were dried over anhydrous sodium sulfate, filtered, and concentrated, the crude was purified by C.C. to give the desired product 0040-2 (400 mg, yield: 46%) as yellow oil.

The synthesis of 2-(4-methoxyphenyl)-2-phenylpropanoic acid (0040-3).

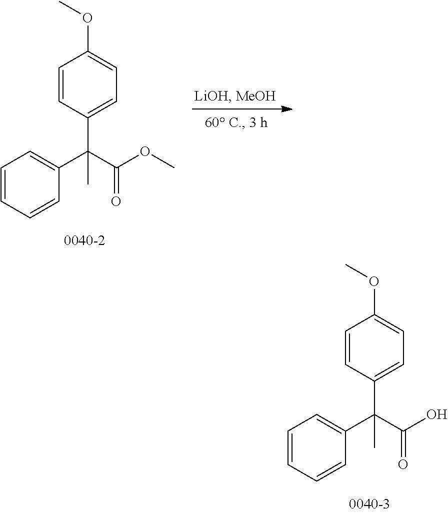 [SMILES: COC(=O)C(C)(c1ccccc1)c1ccc(OC)cc1 ; COc1ccc(C(C)(C(=O)O)c2ccccc2)cc1]

To a stirred solution of 0040-2 (400 mg, 1.5 mmol) in methanol (10 mL) was added LiOH (320 mg, 7.5 mmol) at rt. The resulting reaction mixture was stirred for 16 h at rt. Then added water, the aqueous phase was extracted with EA, the combined organic phases were dried over anhydrous sodium sulfate, filtered, and concentrated in vacuo and purified by prep-HPLC to give the desired product 0040-3 (320 mg, yield: 84%) as a yellow solid.

The synthesis of 2-(4-methoxyphenyl)-2-phenyl-N-propylpropanamide (SU20666-0040).

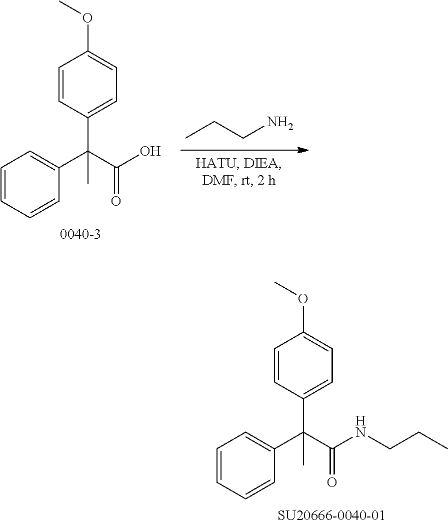

To a solution of compound 0040-3 (100 mg, 0.39 mmol) in DMF (10 mL) was added propan-1-amine (34 mg, 0.58 mmol), DIEA (150 mg, 1.2 mmol) and HATU (220 mg, 0.58 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0040 (53 mg, yield: 46%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.131 min; MS Calcd.: 297.2; MS Found: 298.4 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.259 min.

¹H NMR (400 MHz, DMSO-d₆) δ 0.77 (3H, t, J=7.2 Hz), 1.37-1.42 (2H, m), 1.81 (3H, s), 3.03 (2H, q, J=6.8 Hz), 3.73 (3H, s), 6.86 (2H, d, J=8.8 Hz), 7.08 (2H, d, J=8.8 Hz), 7.14 (2H, d, J=8.8 Hz), 7.18-7.23 (2H, m), 7.27-7.31 (2H, m).

SU20666-0042

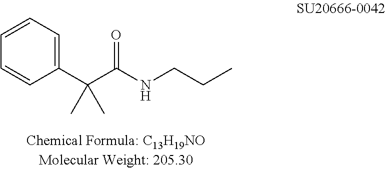

Route for SU20666-0042

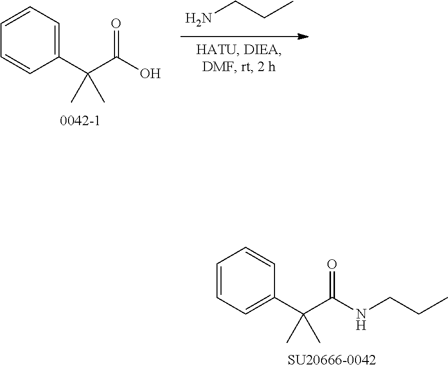

The synthesis of 2-methyl-2-phenyl-N-propylpropanamide (SU20666-0042).

To a solution of compound 0042-1 (200 mg, 1.2 mmol) in DCM (10 mL) was added propan-1-amine (86 mg, 1.5 mmol), DIEA (472 mg, 3.7 mmol) and HATU (695 mg, 1.8 mmol). The resulting reaction mixture was stirred for 2 h at rt, then added water, the aqueous phase was extracted with DCM, the combined organic phases were dried over anhydrous sodium sulfate, filtered, and concentrated, the crude was purified by prep-HPLC to give the desired product SU20666-0042 (70 mg, yield: 28%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.754 min; MS Calcd.: 205.2; MS Found: 206.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water +10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.027 min.

¹H NMR (400 MHz, DMSO-d₆) δ 0.74 (3H, t, J=7.6 Hz), 1.33-1.38 (2H, m), 1.43 (6H, s), 2.98 (2H, q, J=6.8 Hz), 7.18-7.22 (1H, m), 7.28-7.32 (5H, m).

The names SU20666-0043, SP 43, and 43 all refer to the same compound having the formula:

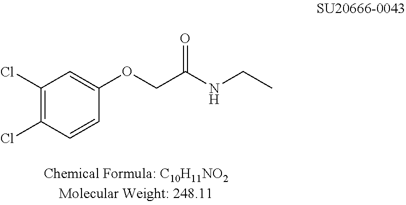

Route for SU20666-0043

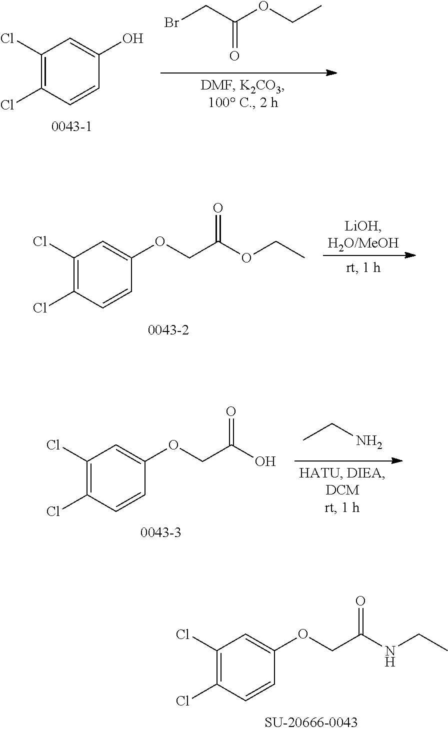

The synthesis of ethyl 2-(3,4-dichlorophenoxy)acetate (0043-2).

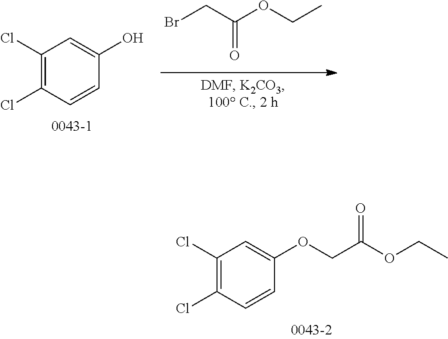

To a stirred solution of 0043-1 (4.0 g, 24.5 mmol) in DMF (40 ml) was added ethyl 2-bromoacetate (4.9 g, 29.4 mmol), Cs₂CO₃ (9.6 g, 29.4 mmol). The resulting reaction mixture was stirred at 100° C. for 12 h. Then added water, the aqueous phase was extracted with EA, the combined organic phases were dried over anhydrous sodium sulfate, filtered, and concentrated in vacuo, further purified by C.C. to give the desired product 0043-2 (6.0 g, yield: 98%) as a yellow solid.

The synthesis of 2-(3,4-dichlorophenoxy)acetic acid (0043-3).

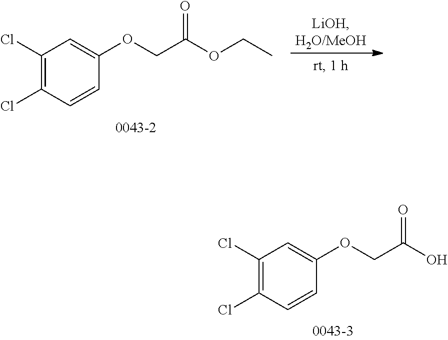

To a stirred solution of 0043-2 (6.0 g, 24.1 mmol) in MeOH/H₂O (40 ml/4 mL) was added LiOH (4.6 g, 120.5 mmol) at rt. The resulting reaction mixture was stirred for 16 h at rt. Then added water, the aqueous phase was extracted with EA, the combined organic phases were dried over anhydrous sodium sulfate, filtered, and concentrated in vacuo and purified by prep-HPLC to give the desired product 0043-3 (4.5 g, yield: 85%) as a yellow solid.

The synthesis of 2-(3,4-dichlorophenoxy)-N-ethylacetamide (SU-20666-0043).

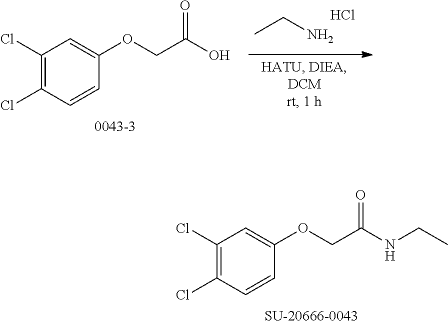

To a solution of compound 0043-3 (200 mg, 0.91 mmol) in DCM (10 mL) was added ethanamine hydrochloride (89 mg, 1.10 mmol), DIEA (348 mg, 2.7 mmol) and HATU (518 mg, 1.4 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0043 (149 mg, yield: 66%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.871 min; MS Calcd.: 247.0; MS Found: 248.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.149 min.

¹H NMR (400 MHz, DMSO-d₆) δ 1.03 (3H, t, J=7.2 Hz), 3.11-3.16 (2H, m), 4.51 (2H, s), 7.99 (1H, dd, J=9.2, 3.2 Hz), 7.26 (1H, d, J=3.2 Hz), 7.55 (1H, d, J=8.8 Hz), 8.13 (1H, s).

The names SU20666-0044, SP 44, and 44 all refer to the same compound having the formula:

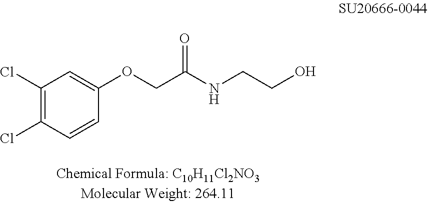 [SMILES: O=C(COc1ccc(Cl)c(Cl)c1)NCCO]

Route for SU20666-0044

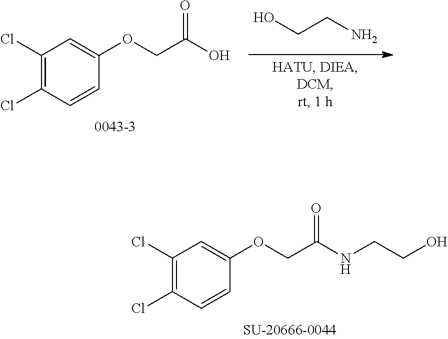

The synthesis of 2-(3,4-dichlorophenoxy)-N-(2-hydroxyethyl)acetamide (SU-20666-0044).

To a solution of compound 0043-3 (200 mg, 0.91 mmol) in DCM (10 mL) was added 2-aminoethanol (67 mg, 1.10 mmol), DIEA (348 mg, 2.7 mmol) and HATU (518 mg, 1.4 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0044 (127 mg, yield: 53%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.29%, Rt=1.607 min; MS Calcd.: 263.0; MS Found: 264.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.602 min.

¹H NMR (400 MHz, DMSO-d₆) δ 3.20 (2H, q, J=6.0 Hz), 3.42 (2H, q, J=6.0 Hz), 4.53 (2H, s), 4.71 (1H, t, J=5.6 Hz), 6.99 (1H, dd, J=8.8, 2.8 Hz), 7.26 (1H, d, J=2.8 Hz), 7.50 (1H, d, J=9.2 Hz), 8.07 (1H, t, J=4.8 Hz).

The names SU20666-0045, SP 45, and 45 all refer to the same compound having the formula:

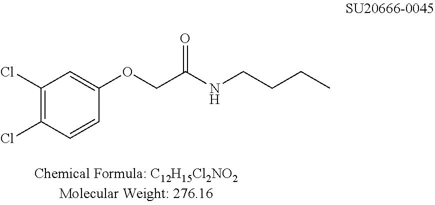 [SMILES: CCCCNC(=O)COc1ccc(Cl)c(Cl)c1]

Route for SU20666-0045

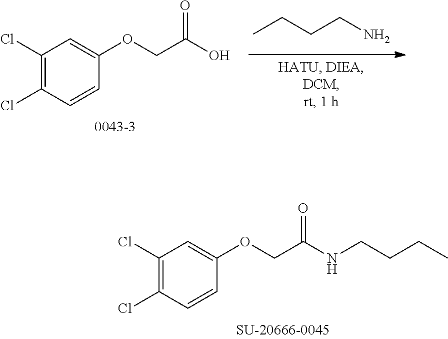

The synthesis of N-butyl-2-(3,4-dichlorophenoxy)acetamide (SU-20666-0045).

To a solution of compound 0043-3 (200 mg, 0.91 mmol) in DCM (10 mL) was added butan-1-amine (80 mg, 1.10 mmol), DIEA (348 mg, 2.7 mmol) and HATU (518 mg, 1.4 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0045 (121 mg, yield: 48%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.073 min; MS Calcd.: 275.1; MS Found: 276.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water +10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.316 min.

¹H NMR (400 MHz, DMSO-d₆) δ 0.86 (2H, t, J=7.2 Hz), 1.22-1.27 (2H, m), 1.36-1.42 (2H, m), 3.11 (2H, q, J=6.8 Hz), 4.52 (2H, s), 6.98 (1H, dd, J=8.8, 2.8 Hz), 7.24 (1H, d, J=2.8 Hz), 7.55 (1H, d, J=8.8 Hz), 8.07 (1H, t, J=5.2 Hz).

The names SU20666-0046, SP 46, and 46 all refer to the same compound having the formula:

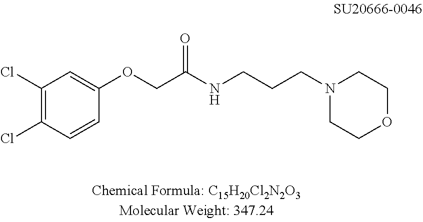 [SMILES: O=C(COc1ccc(Cl)c(Cl)c1)NCCCN1CCOCC1]

Route for SU20666-0046

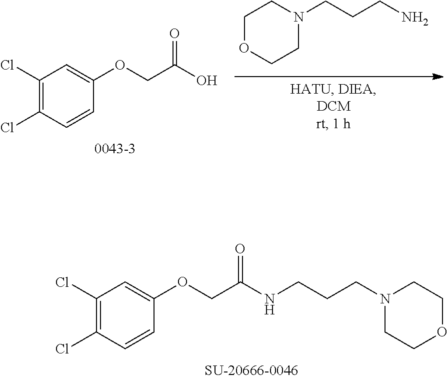 [SMILES: O=C(O)COc1ccc(Cl)c(Cl)c1 ; NCCCN1CCOCC1 ; O=C(COc1ccc(Cl)c(Cl)c1)NCCCN1CCOCC1]

The synthesis of 2-(3,4-dichlorophenoxy)-N-(3-morpholinopropyl)acetamide (SU-20666-0046).

To a solution of compound 0043-3 (150 mg, 0.68 mmol) in DCM (10 mL) was added 3-morpholinopropan-1-amine (144 mg, 0.82 mmol), DIEA (258 mg, 2.0 mmol) and HATU (388 mg, 1.0 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0046 (94 mg, yield: 40%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.734 min; MS Calcd.: 346.1; MS Found: 347.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.293 min.

¹H NMR (400 MHz, DMSO-d₆) δ 1.53-1.61 (2H, m), 2.22 (2H, t, J=7.2 Hz), 2.29-2.33 (4H, m), 3.15 (2H, q, J=6.8 Hz), 3.55 (4H, t, J=4.8 Hz), 4.52 (2H, s), 6.98 (1H, dd, J=8.8, 2.8 Hz), 7.25 (1H, d, J=3.2 Hz), 7.55 (1H, d, J=8.8 Hz), 8.11 (1H, t, J=5.6 Hz).

The names SU20666-0047, SP 47, and 47 all refer to the same compound having the formula:

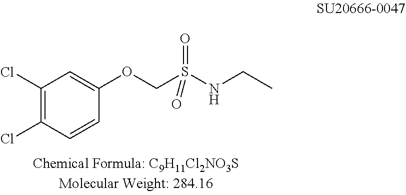 [SMILES: CCNS(=O)(=O)COc1ccc(Cl)c(Cl)c1]

Route for SU20666-0047

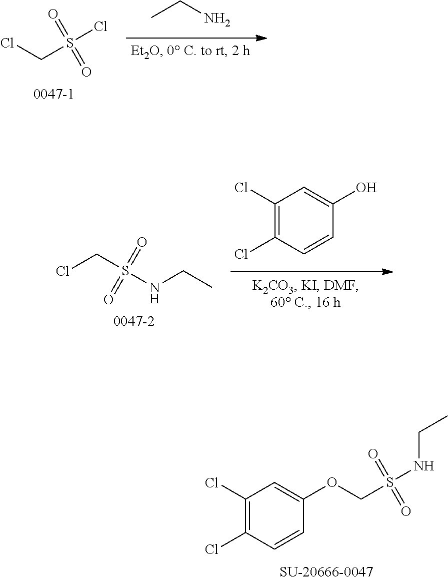

The synthesis of 1-chloro-N-ethylmethanesulfonamide (0047-2).

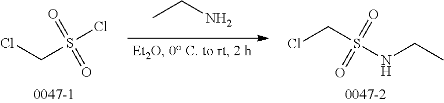

To a solution of compound 0047-1 (1.5 g, 1.0 mmol) in Et₂O (15 mL) was added ethanamine (2.0 M in THF, 12.5 mL, 2.5 mmol) at 0° C. The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product 0047-2 (600 mg, yield: 38%) as colorless oil.

The synthesis of 1-(3,4-dichlorophenoxy)-N-ethylmethanesulfonamide (SU-20666-0047).

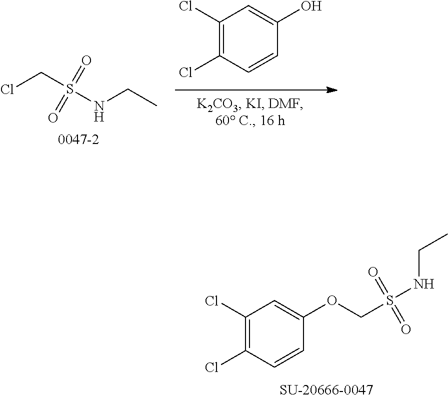

To a stirred solution of 3,4-dichlorophenol (200 mg, 1.2 mmol) in DMF (10 ml) was added 0047-2 (230 mg, 1.5 mmol) and K₂CO₃ (339 mg, 2.5 mmol). The resulting reaction mixture was stirred at 60° C. for 16 h. Then added water, the aqueous phase was extracted with EA, the combined organic phases were dried over anhydrous sodium sulfate, filtered, and concentrated in vacuo, further purified by prep-HPLC to give the desired product SU-20666-0047 (25 mg, yield: 10%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 94.15%, Rt=1.932 min; MS Calcd.: 283.0; MS Found: 282.0 [M−H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.636 min.

¹H NMR (400 MHz, CDCl₃) δ 1.22-1.27 (3H, m), 3.24-3.27 (2H, m), 4.44 (1H, s), 4.97-4.99 (2H, m), 6.92-6.95 (1H, m), 7.17-7.18 (1H, m), 7.37-7.42 (1H, m).

The names SU20666-0051, SP 51, and 51 all refer to the same compound having the formula as shown below.

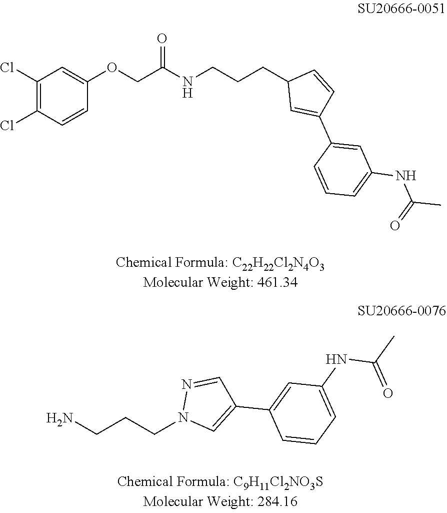 [SMILES: CC(=O)Nc1cccc(C2=CC(CCCNC(=O)COc3ccc(Cl)c(Cl)c3)C=C2)c1 ; CC(=O)Nc1cccc(-c2cnn(CCCN)c2)c1]

Route for SU20666-0051 and SU20666-0076

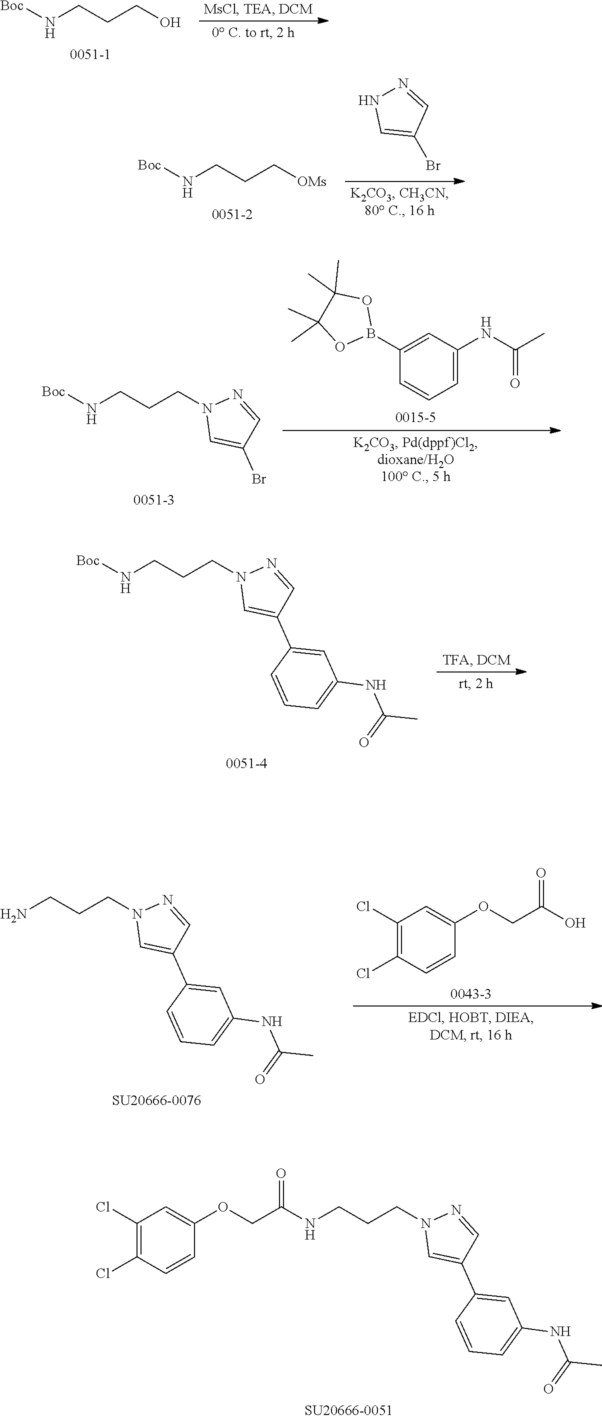

The synthesis of 1-chloro-N-ethylmethanesulfonamide (0051-2).

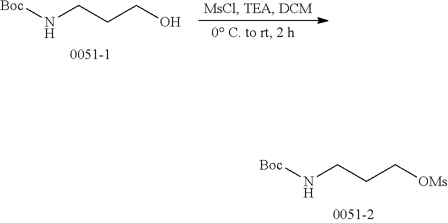 [SMILES: CC(C)(C)OC(=O)NCCCO ; CC(C)(C)OC(=O)NCCCOS(C)(=O)=O]

To a stirred solution of 0051-1 (1.0 g, 5.7 mmol) in DCM (20 ml) was added TEA (1.2 g, 11.4 mmol) and MsCl (0.78 g, 6.8 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo to give the desired product 0051-2 (1.3 g, yield: 90%) as yellow oil.

The synthesis of tert-butyl (3-(4-bromo-1H-pyrazol-1-yl)propyl)carbamate (0051-3).

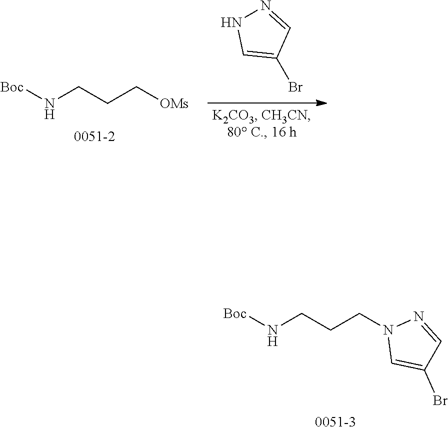 [SMILES: CC(C)(C)OC(=O)NCCCOS(C)(=O)=O ; Brc1cn[nH]c1 ; CC(C)(C)OC(=O)NCCCn1cc(Br)cn1]

To a stirred solution of 4-bromo-1H-pyrazole (0.74 g, 5.1 mmol) in acetonitrile (30 ml) was added 0051-2 (1.3 g, 5.1 mmol) and K₂CO₃ (0.84 g, 6.1 mmol). The resulting reaction mixture was stirred at 80° C. for 16 h. Then added water, the aqueous phase was extracted with EA, the combined organic phases were dried over anhydrous sodium sulfate, filtered, and concentrated in vacuo, further purified by prep-HPLC to give the desired product 0051-3 (0.90 g, yield: 58%) as a yellow solid.

The synthesis of tert-butyl (3-(4-(3-acetamidophenyl)-1H-pyrazol-1-yl)propyl)carbamate (0051-4).

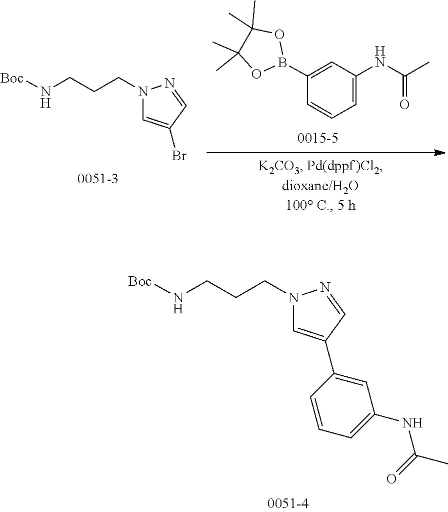 [SMILES: CC(C)(C)OC(=O)NCCCn1cc(Br)cn1 ; CC(=O)Nc1cccc(B2OC(C)(C)C(C)(C)O2)c1 ; CC(=O)Nc1cccc(-c2cnn(CCCNC(=O)OC(C)(C)C)c2)c1]

To a stirred solution of compound 0051-3 (300 mg, 0.99 mmol) in dioxane/water (10 mL/2 mL) was added 0015-5 (260 mg, 0.99 mmol), K₂CO₃ (164 mg, 1.2 mmol), Pd(dppf)Cl₂ (30 mg). The resulting reaction mixture was heated to 100° C. and stirred for 5 h and concentrated in vacuo to remove the solvent, then added water, the aqueous phase was extracted with EA, the combined organic phases were dried over anhydrous sodium sulfate, filtered, and concentrated, the crude was purified by prep-HPLC to give the desired product 0051-4 (150 mg, yield: 36%) as a yellow solid.

The synthesis of N-(3-(1-(3-aminopropyl)-1H-pyrazol-4-yl)phenypacetamide (SU20666-0076).

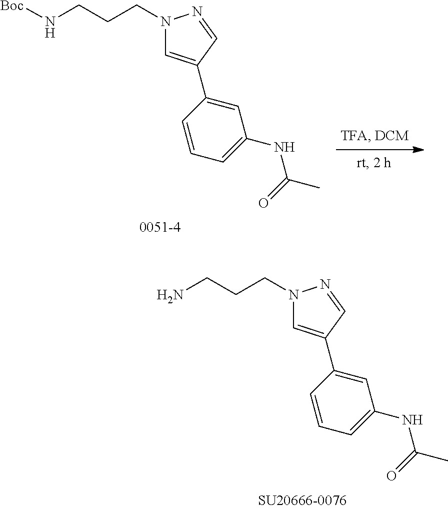 [SMILES: CC(=O)Nc1cccc(-c2cnn(CCCNC(=O)OC(C)(C)C)c2)c1 ; CC(=O)Nc1cccc(-c2cnn(CCCN)c2)c1]

To a stirred solution of compound 0051-4 (150 mg, 0.41 mmol) in DCM (10 mL) was added TFA (3 mL) at rt. The resulting reaction mixture was further stirred for 2 h at rt, then concentrated in vacuo and further purified by prep-HPLC to give the desired product SU20666-0076 (80 mg, yield: 74%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.69%, Rt=1.454 min; MS Calcd.: 258.1; MS Found: 259.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.70%, Rt=5.517 min.

¹H NMR (400 MHz, DMSO-d₆) δ 1.83-1,90 (2H, m), 2.04 (3H, s), 3.32 (2H, s), 4.13-4.19 (2H, m), 4.54 (2H, s), 7.21-7.28 (2H, m), 7.37-7.38 (1H, m), 7.74-7.76 (2H, m), 8.08-8.11 (1H, m), 9.92 (1H, s).

The synthesis of N-(3-(4-(3-acetamidophenyl)-1H-pyrazol-1-yl)propyl)-2-(3,4-dichlorophenoxy)acetamide (SU20666-0051).

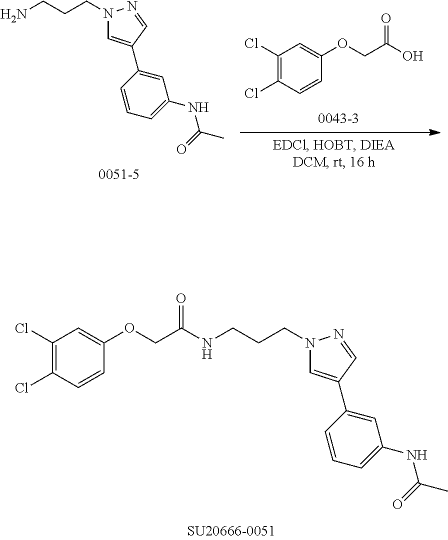 [SMILES: CC(=O)Nc1cccc(-c2cnn(CCCN)c2)c1 ; O=C(O)COc1ccc(Cl)c(Cl)c1 ; CC(=O)Nc1cccc(-c2cnn(CCCNC(=O)COc3ccc(Cl)c(Cl)c3)c2)c1]

To a solution of compound 0051-5 (80 mg, 0.31 mmol) in DCM (10 mL) was added 0043-3 (68 mg, 0.31 mmol), DIEA (190 mg, 1.5 mmol), EDCI (88 mg, 0.46 mmol) and HOBT (62 mg, 0.46 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0051 (40 mg, yield: 24%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.47%, Rt=1.883 min; MS Calcd.: 460.1; MS Found: 461.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.33%, Rt=9.012 min.

¹H NMR (400 MHz, DMSO-d₆) δ 1.92-2.02 (2H, m), 2.04 (3H, s), 3.14 (2H, q, J=6.8 Hz), 4.13 (2H, t, J=6.8 Hz), 4.54 (2H, s), 6.99 (1H, dd, J=9.2, 3.2 Hz), 7.20-7.28 (3H, m), 7.37 (1H, d, J=8.0 Hz), 7.55 (1H, d, J=8.8 Hz), 7.74-7.77 (2H, m), 8.08 (1H, s), 8.20 (1H, t, J=5.6 Hz), 9.92 (1H, s).

The names SU20666-0052, SP 52, and 52 all refer to the same compound having the formula:

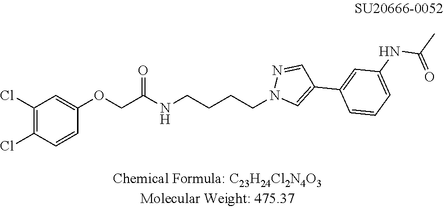 [SMILES: CC(=O)Nc1cccc(-c2cnn(CCCCNC(=O)COc3ccc(Cl)c(Cl)c3)c2)c1]

Scheme 1: Route for SU20666-0052

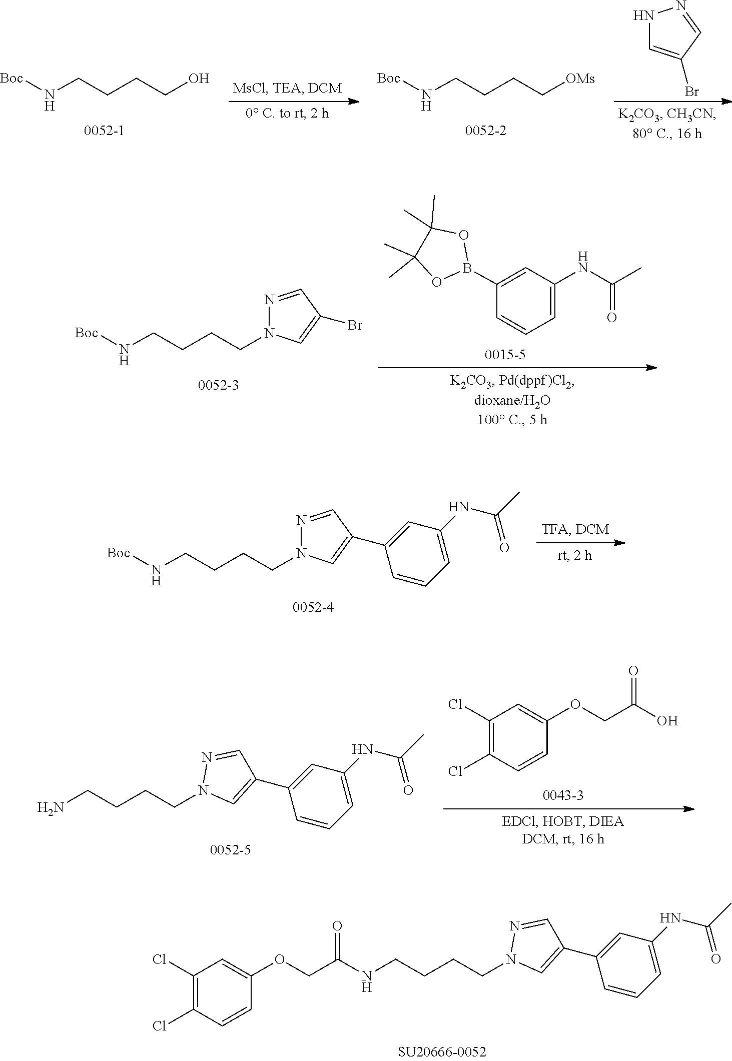

The synthesis of 4-((tert-butoxycarbonyl)amino)butyl methanesulfonate (0052-2).

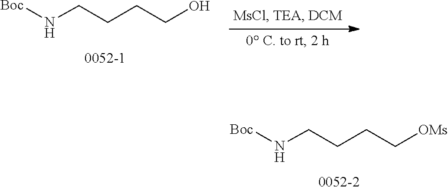 [SMILES: CC(C)(C)OC(=O)NCCCCO ; CC(C)(C)OC(=O)NCCCCOS(C)(=O)=O]

To a stirred solution of 0052-1 (1.0 g, 5.3 mmol) in DCM (30 ml) was added TEA (1.0 g, 10.6 mmol) and MsCl (0.72 g, 6.3 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo to give the desired product 0052-2 (1.2 g, yield: 85%) as yellow oil.

The synthesis of tert-butyl (4-(4-bromo-1H-pyrazol-1-yl)butyl)carbamate (0052-3).

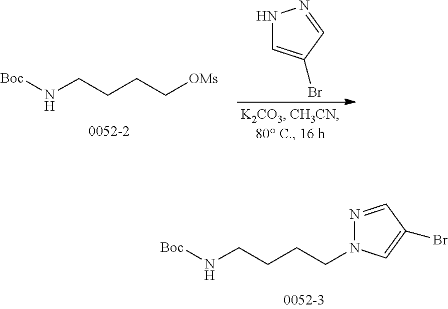 [SMILES: CC(C)(C)OC(=O)NCCCCOS(C)(=O)=O ; Brc1cn[nH]c1 ; CC(C)(C)OC(=O)NCCCCn1cc(Br)cn1]

To a stirred solution of 4-bromo-1H-pyrazole (0.65 g, 4.5 mmol) in acetonitrile (20 ml) was added 0052-2 (1.2 g, 4.5 mmol) and K₂CO₃ (0.93 g, 6.8 mmol). The resulting reaction mixture was stirred at 80° C. for 16 h. Then added water, the aqueous phase was extracted with EA, the combined organic phases were dried over anhydrous sodium sulfate, filtered, and concentrated in vacuo, further purified by prep-HPLC to give the desired product 0052-3 (1.0 g, yield: 70%) as a yellow solid.

The synthesis of tert-butyl (4-(4-(3-acetamidophenyl)-1H-pyrazol-1-yl)butyl)carbamate (0052-4).

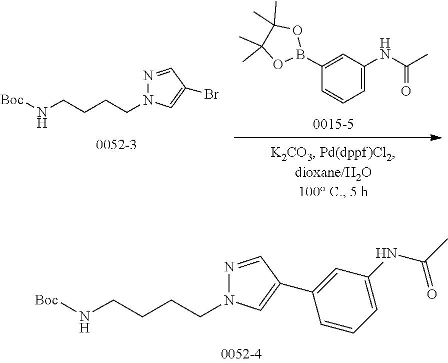 [SMILES: CC(C)(C)OC(=O)NCCCCn1cc(Br)cn1 ; CC(=O)Nc1cccc(B2OC(C)(C)C(C)(C)O2)c1 ; CC(=O)Nc1cccc(-c2cnn(CCCCNC(=O)OC(C)(C)C)c2)c1]

To a stirred solution of compound 0052-3 (300 mg, 0.95 mmol) in dioxane/water (20 mL/2 mL) was added 0015-5 (250 mg, 0.95 mmol), K₂CO₃ (200 mg, 1.4 mmol), Pd(dppf)Cl₂ (30 mg). The resulting reaction mixture was heated to 100° C. and stirred for 5 h and concentrated in vacuo to remove the solvent, then added water, the aqueous phase was extracted with EA, the combined organic phases were dried over anhydrous sodium sulfate, filtered, and concentrated, the crude was purified by prep-HPLC to give the desired product 0052-4 (160 mg, yield: 45%) as a yellow solid.

The synthesis of N-(3-(1-(4-aminobutyl)-1H-pyrazol-4-yl)phenyl)acetamide (0052-5).

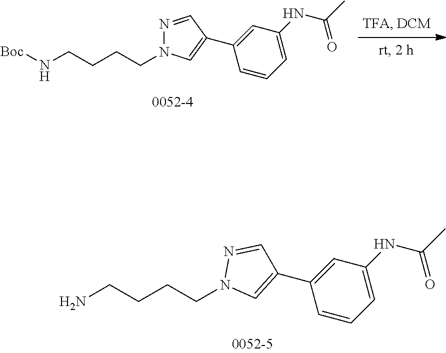 [SMILES: CC(=O)Nc1cccc(-c2cnn(CCCCNC(=O)OC(C)(C)C)c2)c1 ; CC(=O)Nc1cccc(-c2cnn(CCCCN)c2)c1]

To a stirred solution of compound 0052-4 (160 mg, 0.43 mmol) in DCM (10 mL) was added TFA (3 mL) at rt. The resulting reaction mixture was further stirred for 2 h at rt, then concentrated in vacuo to give the desired product 0052-5 (120 mg, yield: 100%) as a yellow solid.

The synthesis of N-(4-(4-(3-acetamidophenyl)-1H-pyrazol-1-yl)butyl)-2-(3,4-dichlorophenoxy)acetamide (SU20666-0052).

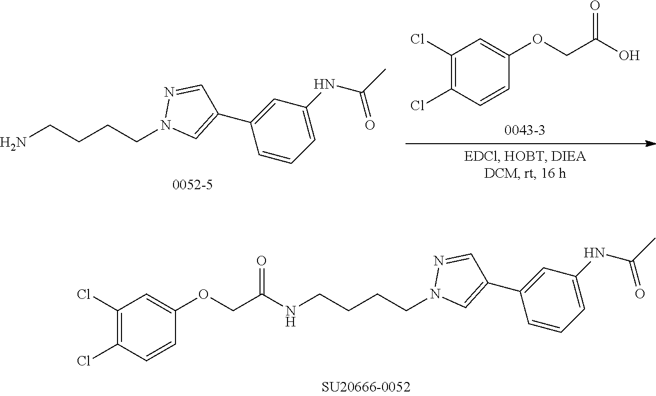 [SMILES: CC(=O)Nc1cccc(-c2cnn(CCCCN)c2)c1 ; O=C(O)COc1ccc(Cl)c(Cl)c1 ; CC(=O)Nc1cccc(-c2cnn(CCCCNC(=O)COc3ccc(Cl)c(Cl)c3)c2)c1]

To a solution of compound 0052-5 (110 mg, 0.40 mmol) in DCM (20 mL) was added 0043-3 (89 mg, 0.40 mmol), DIEA (260 mg, 2.0 mmol), EDCI (115 mg, 0.60 mmol) and HOBT (82 mg, 0.60 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0052 (48 mg, yield: 25%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.62%, Rt=1.770 min; MS Calcd.: 474.1; MS Found: 475.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.045 min.

¹H NMR (400 MHz, DMSO-d₆) δ 1.38-1.42 (2H, m), 1.74-1.79 (2H, m), 2.04 (3H, s), 3.15 (2H, q, J=8.8 Hz), 4.12 (2H, t, J=6.8 Hz), 4.52 (2H, s), 6.97 (1H, dd, J=8.8, 2.8 Hz), 7.20-7.28 (3H, m), 7.37 (1H, d, J=8.0 Hz), 7.53 (1H, d, J=8.8 Hz), 7.75 (2H, s), 8.07 (1H, s), 8.16 (1H, t, J=6.0 Hz), 9.92 (1H, s).

The names SU20666-0053, SP 53, and 53 all refer to the same compound having the formula as shown below. The names SU20666-0054, SP 54, and 54 all refer to the same compound having the formula as shown below. The names SU20666-0064, SP 64, and 64 all refer to the same compound having the formula as shown below.

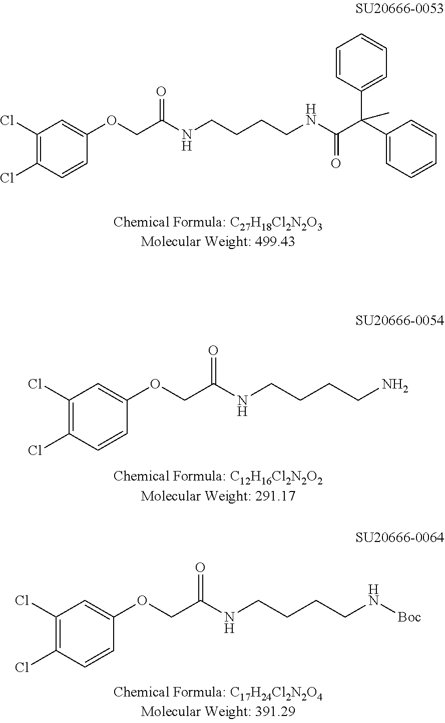 [SMILES: CC(C(=O)NCCCCNC(=O)COc1ccc(Cl)c(Cl)c1)(c1ccccc1)c1ccccc1 ; NCCCCNC(=O)COc1ccc(Cl)c(Cl)c1 ; CC(C)(C)OC(=O)NCCCCNC(=O)COc1ccc(Cl)c(Cl)c1]

Route for SU20666-0053, SU20666-0054 and SU20666-0064

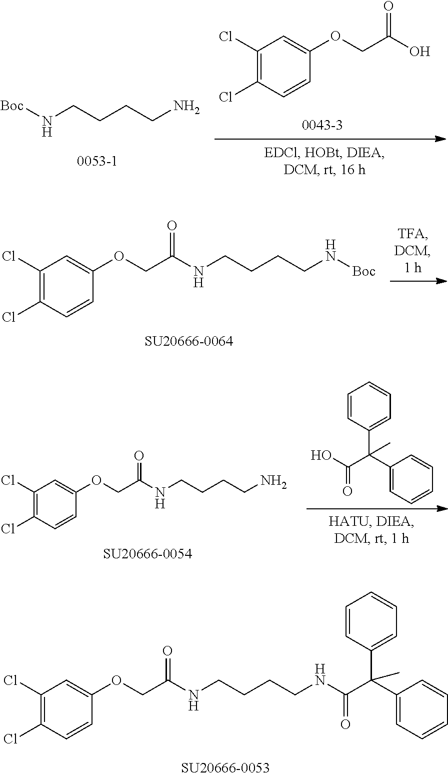 [SMILES: O=C(O)COc1ccc(Cl)c(Cl)c1 ; CC(C)(C)OC(=O)NCCCCN ; CC(C)(C)OC(=O)NCCCCNC(=O)COc1ccc(Cl)c(Cl)c1 ; NCCCCNC(=O)COc1ccc(Cl)c(Cl)c1 ; CC(C(=O)O)(c1ccccc1)c1ccccc1 ; CC(C(=O)NCCCCNC(=O)COc1ccc(Cl)c(Cl)c1)(c1ccccc1)c1ccccc1]

The synthesis of tert-butyl (4-(2-(3,4-dichlorophenoxy)acetamido)butyl)carbamate (SU20666-0064).

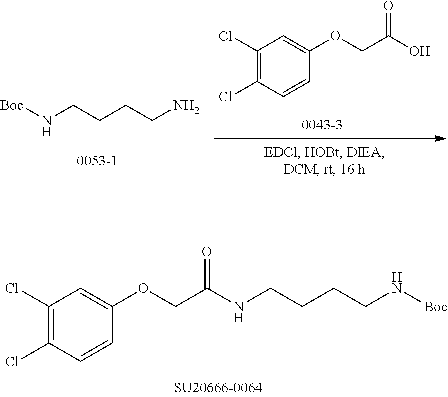 [SMILES: O=C(O)COc1ccc(Cl)c(Cl)c1 ; CC(C)(C)OC(=O)NCCCCN ; CC(C)(C)OC(=O)NCCCCNC(=O)COc1ccc(Cl)c(Cl)c1]

To a solution of compound 0053-1 (390 mg, 1.8 mmol) in DCM (20 mL) was added 0043-3 (400 mg, 2.1 mmol), DIEA (684 mg, 5.3 mmol), EDCI (510 mg, 2.7 mmol) and HOBT (362 mg, 2.6 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0064 (500 mg, yield: 58%) as colorless oil.

LC-MS (Agilent LCMS 1200-6120, Column: Waters X-Bridge C18 (50 mm*4.6 mm*3.5μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.49%, Rt=2.102 min; MS Calcd.: 390.1; MS Found: 335.0 [M−56]⁺ and 291.0 [M−100]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.446 min.

¹H NMR (400 MHz, DMSO-d₆) δ 1.32-1.34 (2H, m), 1.37 (9H, s), 1.37-1.39 (2H, m), 2.87-2.91 (2H, m), 3.10 (1H, q, J=6.4 Hz), 4.51 (2H, s), 6.78 (1H, t, J=5.6 Hz), 6.98 (1H, dd, J=9.2, 2.8 Hz), 7.25 (1H, d, J=2.8 Hz), 7.55 (1H, d, J=8.8 Hz), 8.11 (1H, t, J=5.6 Hz).

The synthesis of N-(4-aminobutyl)-2-(3,4-dichlorophenoxy)acetamide (SU20666-0054).

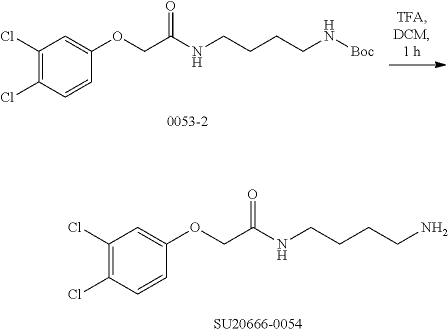 [SMILES: CC(C)(C)OC(=O)NCCCCNC(=O)COc1ccc(Cl)c(Cl)c1 ; NCCCCNC(=O)COc1ccc(Cl)c(Cl)c1]

To a stirred solution of compound 0053-2 (400 mg, 1.03 mmol) in DCM (10 mL) was added TFA (3 mL) at rt. The resulting reaction mixture was further stirred for 1 h at rt, then concentrated in vacuo and purified by prep-HPLC to give the desired product SU20666-0054 (200 mg, yield: 35%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.49%, Rt=1.521 min; MS Calcd.: 290.1; MS Found: 291.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.93%, Rt=6.945 min.

¹H NMR (400 MHz, DMSO-d₆) δ 1.23-1.47 (6H, m), 3.10 (2H, q, J=6.4 Hz), 4.52 (2H, s), 6.98 (1H, dd, J=8.8, 2.8 Hz), 7.25 (1H, d, J=2.4 Hz), 7.55 (1H, d, J=8.8 Hz), 8.16 (1H, t, J=5.6 Hz).

The synthesis of N-(4-(2-(3,4-dichlorophenoxy)acetamido)butyl)-2,2-diphenylpropanamide (SU20666-0053).

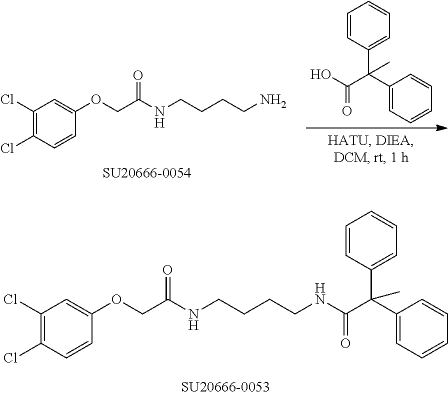 [SMILES: NCCCCNC(=O)COc1ccc(Cl)c(Cl)c1 ; CC(C(=O)O)(c1ccccc1)c1ccccc1 ; CC(C(=O)NCCCCNC(=O)COc1ccc(Cl)c(Cl)c1)(c1ccccc1)c1ccccc1]

To a solution of compound SU20666-0054 (85 mg, 0.29 mmol) in DCM (20 mL) was added 2,2-diphenylpropanoic acid (55 mg, 0.24 mmol), DIEA (155 mg, 1.2 mmol), and HATU (140 mg, 0.37 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0053 (26 mg, yield: 25%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.73%, Rt=2.011 min; MS Calcd.: 498.1; MS Found: 499.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.01%, Rt=11.053 min.

¹H NMR (400 MHz, DMSO-d₆) δ 1.37 (4H, brs.), 1.84 (3H, s), 3.05-3.11 (4H, m), 4.51 (2H, s), 6.98 (1H, dd, J=8.8, 2.8 Hz), 7.14-7.16 (4H, m), 7.20-7.31 (8H, m), 7.54 (1H, d, J=8.8 Hz), 8.11 (1H, t, J=5.6 Hz).

The names SU20666-0055, SP 55, and 55 all refer to the same compound having the formula:

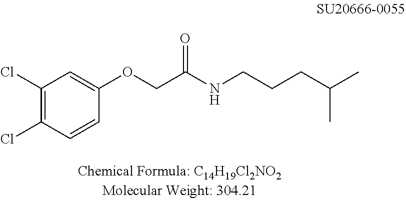 [SMILES: CC(C)CCCNC(=O)COc1ccc(Cl)c(Cl)c1]

Route for SU20666-0055

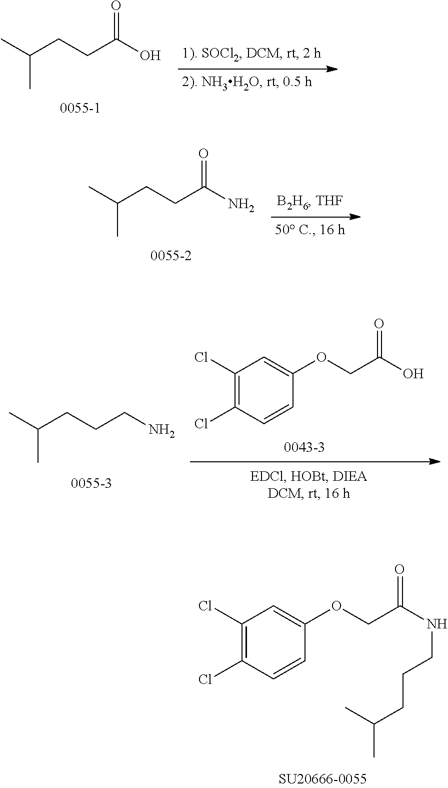

The synthesis of 4-methylpentanamide (0055-2).

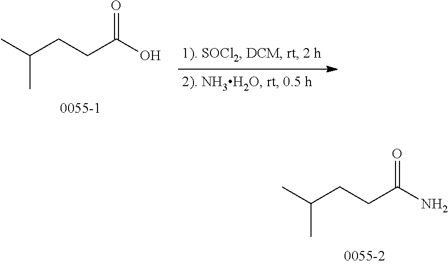

To a solution of compound 0055-1 (500 mg, 4.3 mmol) in DCM (20 mL) was added thionyl chloride (1.0 g, 8.6 mmol). The resulting reaction mixture was stirred for 2 h at rt and concentrated in vacuo, then added ammonium hydroxide (5 mL) and stirred at rt for another 0.5 h, the aqueous phase was extracted with EA, the combined organic phases were dried over anhydrous sodium sulfate, filtered, and concentrated, to give the desired product 0055-2 (240 mg, yield: 48%) as a white solid.

The synthesis of 4-methylpentan-1-amine (0055-3).

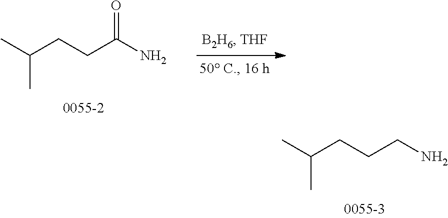

To a stirred solution of 0055-2 (220 mg, 1.9 mmol) in THF (5 ml) was added borane-tetrahydrofuran (1.0 N, 11.5 mL, 11.5 mmol). The resulting reaction mixture was heated to 50° C. and stirred for 16 h. Then added HCl (1.0 N, 5 mL) and stirred for 1 h at rt, the aqueous phase was neutralized and then extracted with dichloromethane, the combined organic phases were dried over anhydrous sodium sulfate, filtered, and concentrated in vacuo to give the desired product 0055-3 (110 mg, yield: 56%) as colorless oil.

The synthesis of 2-(3,4-dichlorophenoxy)-N-(4-methylpentyl)acetamide (SU20666-0055).

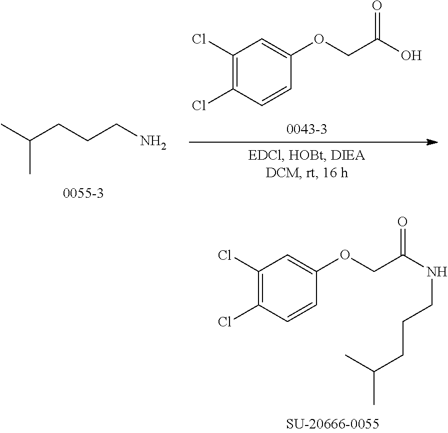

To a solution of compound 0055-3 (200 mg, 0.91 mmol) in DCM (10 mL) was added 0043-3 (110 mg, 1.1 mmol), DIEA (348 mg, 2.7 mmol), EDCI (262 mg, 1.4 mmol) and HOBT (186 mg, 1.4 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0055 (26 mg, yield: 9.5%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.310 min; MS Calcd.: 303.1; MS Found: 304.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.01%, Rt=11.649 min.

¹H NMR (400 MHz, CDCl₃) δ 0.87 (6H, d, J=6.8 Hz), 1.17-1.21 (2H, m), 1.51-1.57 (3H, m), 3.33 (2H, q, J=6.8 Hz), 4.46 (2H, s), 6.45 (1H, brs.), 6.79 (1H, dd, J=8.8, 2.8 Hz), 7.05 (1H, d, J=2.8 Hz), 7.38 (1H, d, J=8.8 Hz).

The names SU20666-0056, SP 56, and 56 all refer to the same compound having the formula:

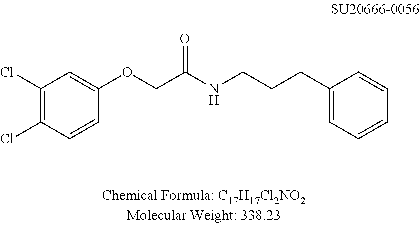 [SMILES: O=C(COc1ccc(Cl)c(Cl)c1)NCCCc1ccccc1]

Route for SU20666-0056

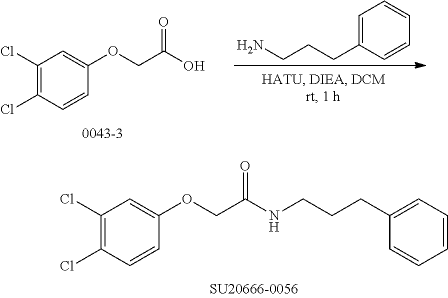 [SMILES: O=C(O)COc1ccc(Cl)c(Cl)c1 ; NCCCc1ccccc1 ; O=C(COc1ccc(Cl)c(Cl)c1)NCCCc1ccccc1]

The synthesis of 2-(3,4-dichlorophenoxy)-N-(3-phenylpropyl)acetamide (SU20666-0056).

To a solution of compound 0043-3 (150 mg, 0.68 mmol) in DCM (5 mL) was added 3-phenylpropan-1-amine (110 mg, 0.82 mmol), DIEA (258 mg, 2.0 mmol) and HATU (388 mg, 1.0 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0056 (42 mg, yield: 18%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.194 min; MS Calcd.: 337.1; MS Found: 338.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.940 min.

¹H NMR (400 MHz, CDCl₃) δ 1.86-1,93 (2H, m), 2.66 (2H, t, J=7.2 Hz), 3.39 (2H, q, J=6.8 Hz), 4.42 (2H, s), 6.42 (1H, brs.), 6.76 (1H, dd, J=8.8, 2.8 Hz), 7.03 (1H, d, J=2.8 Hz), 7.15-7.20 (3H, m), 7.28-7.30 (2H, m), 7.37 (1H, d, J=8.8 Hz).

SU20666-0058 and SU20666-0063

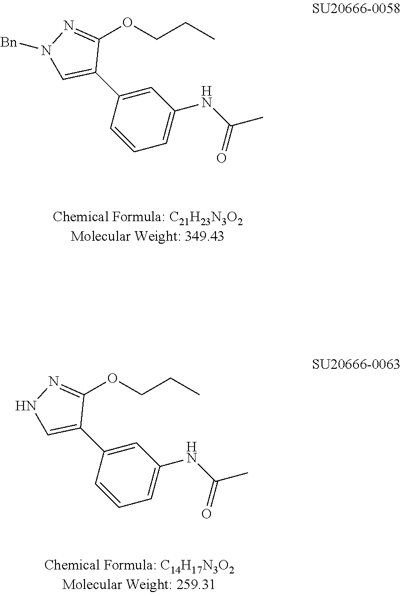 [SMILES: CCCOc1nn(Cc2ccccc2)cc1-c1cccc(NC(C)=O)c1 ; CCCOc1n[nH]cc1-c1cccc(NC(C)=O)c1]

Route for SU20666-0058 and SU20666-0063

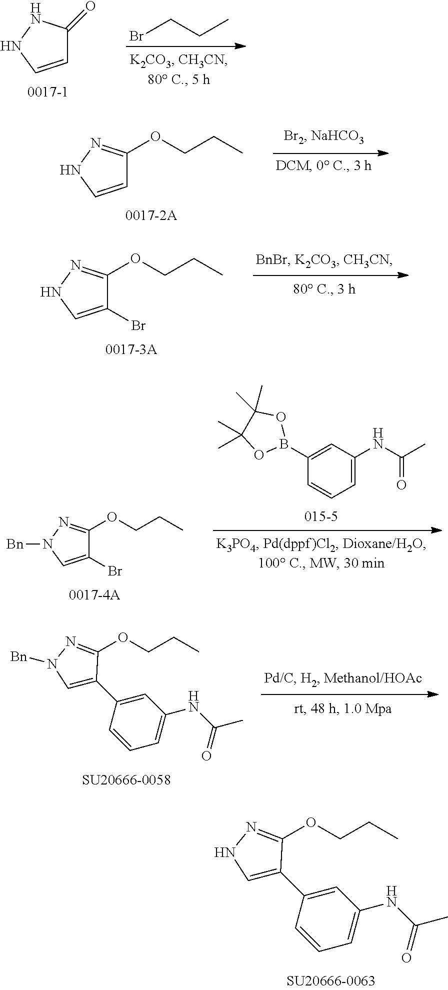

The synthesis of 3-propoxy-1H-pyrazole (0017-2A).

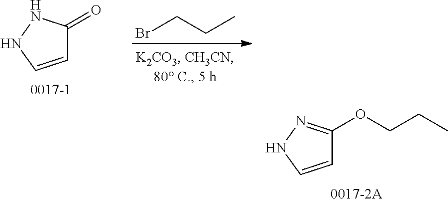

To a stirred solution of 0017-1 (1.5 g, 17.9 mmol) in CH₃CN (50 ml) was added 1-bromopropane (2.2 g, 17.9 mmol), K₂CO₃ (2.7 g, 19.6 mmol). The resulting reaction mixture was heated to 80° C. for 5 h. Then added water, the aqueous phase was extracted with dichloromethane, the combined organic phases were dried over anhydrous sodium sulfate, filtered, and concentrated in vacuopurified by C.C. to give the desired product 0017-2A (1.0 g, yield: 44%) as a yellow solid.

The synthesis of 4-bromo-3-propoxy-1H-pyrazole (0017-3A).

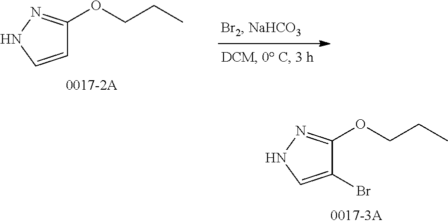

To a stirred solution of 0017-2A (0.30 g, 2.4 mmol) in DCM (20 ml) was added NaHCO₃ (0.24 g, 2.8 mmol) and Br₂ (0.42 g, 2.6 mmol) slowly. The resulting reaction mixture was stirred at 0° C. for 3 h. Then added water, the aqueous phase was extracted with dichloromethane, the combined organic phases were dried over anhydrous sodium sulfate, filtered, and concentrated to give the desired product 0017-3A (0.42 g, yield: 86%) as a yellow solid.

The synthesis of 1-benzyl-4-bromo-3-propoxy-1H-pyrazole (0017-4A).

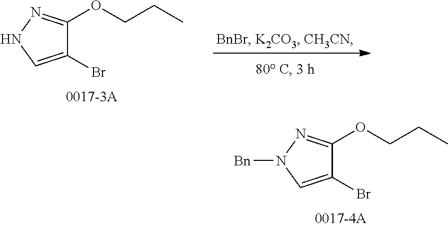

To a stirred solution of 0017-3A (0.30 g, 1.46 mmol) in CH₃CN (20 ml) was added K₂CO₃ (0.22 g, 1.6 mmol) and BnBr (0.28 g, 1.6 mmol). The resulting reaction mixture was heated to 80° C. for 3 h. Then added water, the aqueous phase was extracted with dichloromethane, the combined organic phases were dried over anhydrous sodium sulfate, filtered, and concentrated to give the desired product 0017-4 (0.40 g, yield: 93%) as a yellow solid.

The synthesis of N-(3-(1-benzyl-3-propoxy-1H-pyrazol-4-yl)phenyl)acetamide (SU20666-0058).

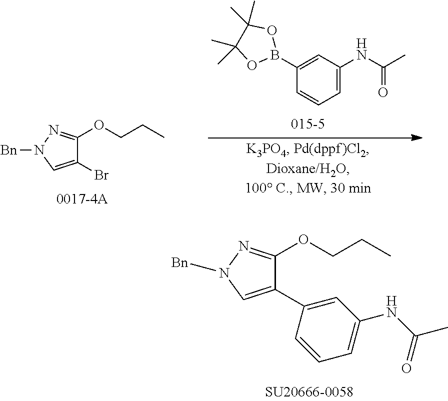 [SMILES: CC(=O)Nc1cccc(B2OC(C)(C)C(C)(C)O2)c1 ; CCCOc1nn(Cc2ccccc2)cc1Br ; CCCOc1nn(Cc2ccccc2)cc1-c1cccc(NC(C)=O)c1]

To a solution of compound 0017-4A (150 mg, 0.51 mmol) in dioxane/water (6 mL/1 mL) was added 015-5 (146 mg, 0.56 mmol), K₃PO₄ (200 mg, 0.76 mmol), Pd(dppf)Cl₂ (30 mg). The resulting reaction mixture was heated to 100° C. and stirred for 0.5 h at MW conditions, then concentrated in vacuo to remove the solvent and added water, the aqueous phase was extracted with dichloromethane, the combined organic phases were dried over anhydrous sodium sulfate, filtered, and concentrated, the crude was purified by prep-TLC to give the desired product SU20666-0058 (20 mg, yield: 11%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.869 min; MS Calcd.: 349.2; MS Found: 350.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.47%, Rt=10.083 min.

¹H NMR (400 MHz, CDCl₃) δ 0.98 (3H, t, J=7.6 Hz), 1.76-1.81 (2H, m), 2.09 (3H, s), 4.18 (2H, t, J=6.8 Hz), 5.07 (2H, s), 7.15-7.33 (9H, m), 7.37 (1H, s), 7.66 (1H, s).

The synthesis of N-(3-(3-propoxy-1H-pyrazol-4-yl)phenyl)acetamide (SU20666-0063).

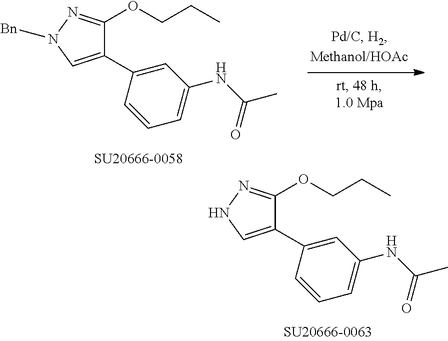 [SMILES: CCCOc1nn(Cc2ccccc2)cc1-c1cccc(NC(C)=O)c1 ; CCCOc1n[nH]cc1-c1cccc(NC(C)=O)c1]

To a stirred solution of compound SU20666-0058 (45 mg, 0.13 mmol) in methanol/HOAc (10 mL/2 mL) was added Pd/C (10%, 20 mg). The resulting reaction mixture was stirred for 48 h at rt under H₂ atomosphere (1.0 Mpa), and then filtered, the filtrate was concentrated in vacuo to remove the solvent and further purified by prep-HPLC to give the desired product SU20666-0063 (13 mg, yield: 39%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.577 min; MS Calcd.: 259.1; MS Found: 260.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=7.539 min.

¹H NMR (400 MHz, DMSO-d₆) δ 1.00 (3H, t, J=7.2 Hz), 1.75-1.80 (2H, m), 2.04 (3H, s), 4.16 (2H, t, J=6.4 Hz), 7.20-7,24 (1H, m), 7.30-7.35 (2H, m), 7.88 (2H, d, J=13.2 Hz), 9.85 (1H, s), 12.01 (1H, s).

The names SU20666-0059, SP 59, and 59 all refer to the same compound having the formula:

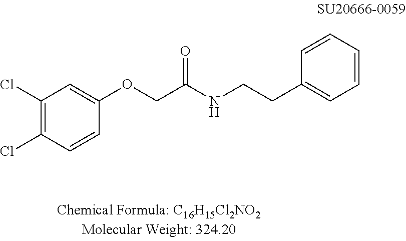 [SMILES: O=C(COc1ccc(Cl)c(Cl)c1)NCCc1ccccc1]

Route for SU20666-0059

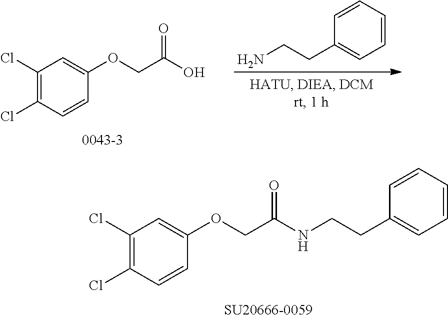 [SMILES: O=C(O)COc1ccc(Cl)c(Cl)c1 ; NCCc1ccccc1 ; O=C(COc1ccc(Cl)c(Cl)c1)NCCc1ccccc1]

The synthesis of 2-(3,4-dichlorophenoxy)-N-phenethylacetamide (SU20666-0059).

To a solution of compound 0043-3 (150 mg, 0.68 mmol) in DCM (5 mL) was added 2-phenylethanamine (100 mg, 0.82 mmol), DIEA (258 mg, 2.0 mmol) and HATU (388 mg, 1.0 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0059 (155 mg, yield: 70%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.193 min; MS Calcd.: 323.1; MS Found: 324.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.964 min.

¹H NMR (400 MHz, CDCl₃) δ 2.84 (2H, t, J=7.2 Hz), 3.61 (2H, q, J=6.8 Hz), 4.43 (2H, s), 6.46 (1H, brs.), 6.69 (1H, dd, J=8.8, 2.8 Hz), 6.96 (1H, d, J=2.4 Hz), 7.14-7.16 (2H, m), 7.22-7.24 (1H, m), 7.31-7.36 (3H, m).

The names SU20666-0060, SP 60, and 60 all refer to the same compound having the formula:

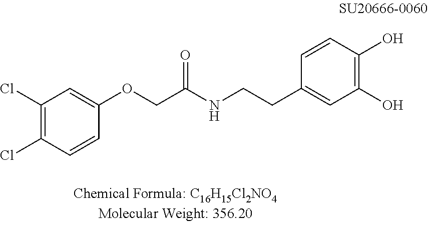 [SMILES: O=C(COc1ccc(Cl)c(Cl)c1)NCCc1ccc(O)c(O)c1]

Route for SU20666-0060

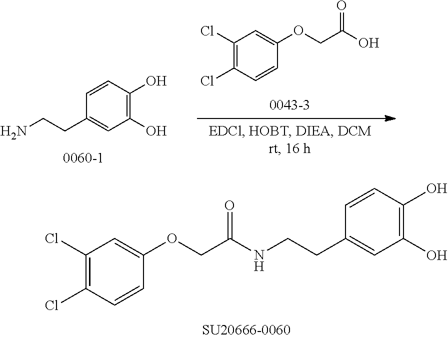 [SMILES: O=C(O)COc1ccc(Cl)c(Cl)c1 ; NCCc1ccc(O)c(O)c1 ; O=C(COc1ccc(Cl)c(Cl)c1)NCCc1ccc(O)c(O)c1]

The synthesis of 2-(3,4-dichlorophenoxy)-N-(3,4-dihydroxyphenethyl)acetamide (SU20666-0060).

To a solution of compound 0060-1 (86 mg, 0.45 mmol) in DCM (10 mL) was added 0043-3 (100 mg, 0.45 mmol), DIEA (174 mg, 1.35 mmol), EDCI (128 mg, 0.67 mmol) and HOBT (91 mg, 0.67 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0060 (60 mg, yield: 32%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.674 min; MS Calcd.: 355.0; MS Found: 356.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 96.78%, Rt=8.331 min.

¹H NMR (400 MHz, DMSO-d₆) δ 2.54-2.56 (2H, m), 3.23-3.28 (2H, m), 4.51 (2H, s), 6.42 (1H, d, J=7.6 Hz), 6.58-6.63 (2H, m), 6.95 (1H, dd, J=8.8, 2.8 Hz), 7.25 (1H, d, J=2.8 Hz), 7.54 (1H, d, J=8.8 Hz), 8.14 (1H, t, J=5.2 Hz), 8.65 (1H, brs.), 8.76 (1H, brs.).

The names SU20666-0061, SP 61, and 61 all refer to the same compound having the formula:

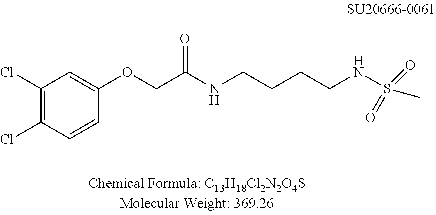 [SMILES: CS(=O)(=O)NCCCCNC(=O)COc1ccc(Cl)c(Cl)c1]

Route for SU20666-0061

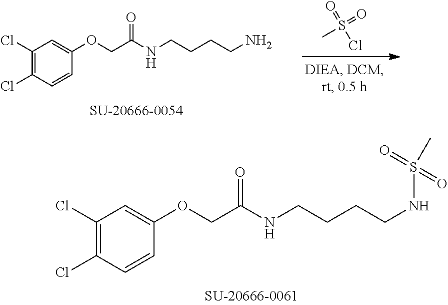

The synthesis of 2-(3,4-dichlorophenoxy)-N-(4-(methylsulfonamido)butyl)acetamide (SU20666-0061).

To a solution of compound SU20666-0054 (100 mg, 0.35 mmol) in DCM (5 mL) was added methanesulfonyl chloride (59 mg, 0.52 mmol) and DIEA (89 mg, 0.69 mmol) at 0° C. The resulting reaction mixture was stirred for 0.5 h at rt, then added water, the aqueous phase was extracted with DCM, the combined organic phases were dried over anhydrous sodium sulfate, filtered, and concentrated, the crude was purified by prep-HPLC to give the desired product SU20666-0061 (54 mg, yield: 42%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.57%, Rt=1.785 min; MS Calcd.: 368.0; MS Found: 369.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.57%, Rt=8.602 min.

¹H NMR (400 MHz, CDCl₃) δ 1.63-1.69 (4H, m), 2.96 (3H, s), 3.18 (2H, q, J=6.4 Hz), 3.39 (2H, q, J=6.4 Hz), 4.46 (2H, s), 4.54(1H, t, J=5.6 Hz), 6.59 (1H, s), 6.80 (1H, dd, J=8.8, 2.8 Hz), 7.05 (1H, d, J=3.2 Hz), 7.38 (1H, d, J=9.2 Hz).

The names SU20666-0062, SP 62, and 62 all refer to the same compound having the formula:

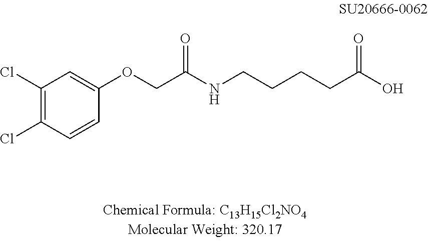 [SMILES: O=C(O)CCCCNC(=O)COc1ccc(Cl)c(Cl)c1]

Route for SU20666-0062

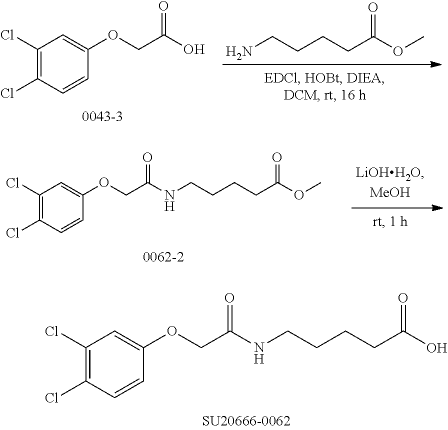

The synthesis of methyl 5-(2-(3,4-dichlorophenoxy)acetamido)pentanoate (0062-2).

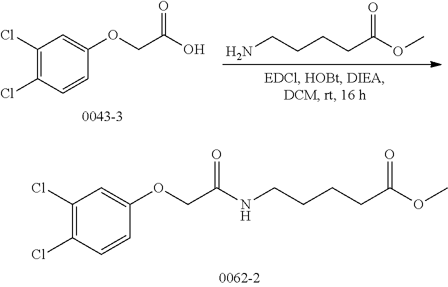

To a solution of compound 0043-3 (250 mg, 1.14 mmol) in DCM (10 mL) was added methyl 5-aminopentanoate (230 mg, 1.36 mmol), DIEA (732 mg, 5.68 mmol), EDCI (436 mg, 2.27 mmol) and HOBT (309 mg, 2.27 mmol) at 0° C. The resulting reaction mixture was stirred for 16 h at rt, then added water, the aqueous phase was extracted with DCM, the combined organic phases were dried over anhydrous sodium sulfate, filtered, and concentrated, the crude was purified by prep-HPLC to give the desired product 0062-2 (250 mg, yield: 76%) as yellow oil.

The synthesis of 5-(2-(3,4-dichlorophenoxy)acetamido)pentanoic acid (SU20666-0062).

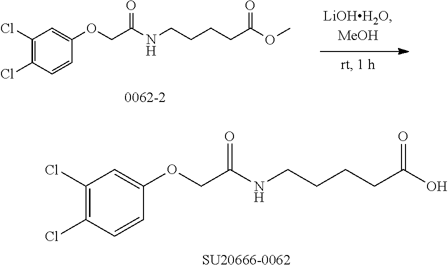 [SMILES: COC(=O)CCCCNC(=O)COc1ccc(Cl)c(Cl)c1 ; O=C(O)CCCCNC(=O)COc1ccc(Cl)c(Cl)c1]

To a stirred solution of 0062-2 (250 mg, 0.75 mmol) in methanol (10 mL) was added LiOH (210 mg, 5.0 mmol) at rt. The resulting reaction mixture was stirred for 1 h at rt. Then added water, the aqueous phase was extracted with EA, the combined organic phases were dried over anhydrous sodium sulfate, filtered, and concentrated in vacuo and purified by prep-HPLC to give the desired product SU20666-0062 (33 mg, yield: 14%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.07%, Rt=1.394 min; MS Calcd.: 319.0; MS Found: 320.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 96.96%, Rt=6.593 min.

¹H NMR (400 MHz, MeOD) 6 1.60-1.61 (4H, m), 2.72-2.30 (2H, m), 3.30-3.31 (2H, m), 4.53 (2H, d, J=1.2 Hz), 6.96-6.99 (1H, m), 7.21-7.22 (1H, m), 7.45 (1H, dd, J=9.2, 1.6 Hz).

The names SU20666-0065, SP 65, and 65 all refer to the same compound having the formula:

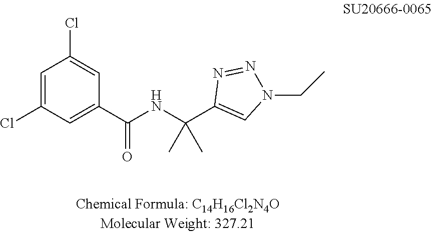 [SMILES: CCn1cc(C(C)(C)NC(=O)c2cc(Cl)cc(Cl)c2)nn1]

Route for SU20666-0065

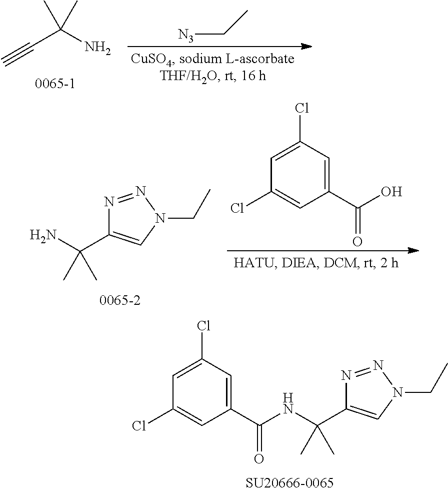

The synthesis of 2-(1-ethyl-1H-1,2,3-triazol-4-yl)propan-2-amine (0065-2).

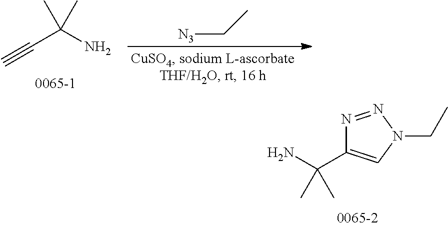

To a solution of compound 0065-1 (300 mg, 3.60 mmol) in THF/H₂O (20 mL/4 mL) was added copper sulfate pentahydrate (440 mg, 1.80 mmol), sodium L-ascorbate (350 mg, 1.80 mmol) and azidoethane (300 mg, 4.30 mmol) at 0° C. The resulting reaction mixture was stirred for 16 h at rt, then added water, the aqueous phase was extracted with DCM, then the aqueous phases was concentrated to give the desired product 0065-2 (400 mg, yield: 73%) as yellow oil, which used to the next step without further purification.

The synthesis of 3,5-dichloro-N-(2-(1-ethyl-1H-1,2,3-triazol-4-yl)propan-2-yl)benzamide (SU20666-0065).

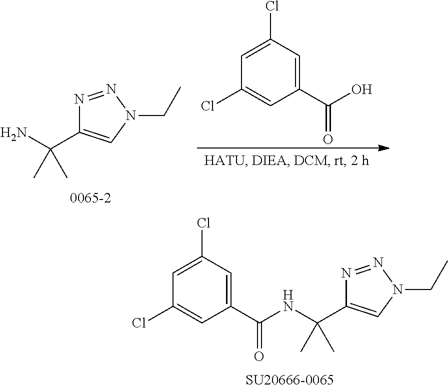

To a solution of compound 0065-2 (100 mg, 0.65 mmol) in DCM (10 mL) was added 3,5-dichlorobenzoic acid (124 mg, 0.65 mmol), DIEA (250 mg, 1.95 mmol) and HATU (380 mg, 0.98 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0065 (41 mg, yield: 19%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.18%, Rt=1.970 min; MS Calcd.: 326.1; MS Found: 327.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 97.40%, Rt=9.742 min.

¹H NMR (400 MHz, DMSO-d₆) δ 1.41 (3H, t, J=7.2 Hz), 1.69 (6H, s), 4.32 (2H, q, J=7.2 Hz), 7.79 (1H, t, J=2.0 Hz), 7.85 (2H, d, J=2.0 Hz), 7.94 (1H, s), 8.59 (1H, s).

The names SU20666-0066, SP 66, and 66 all refer to the same compound having the formula:

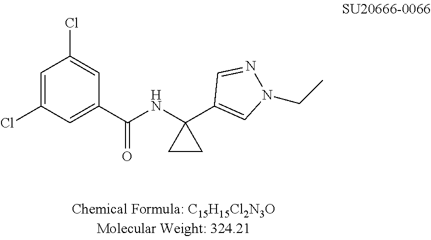 [SMILES: CCn1cc(C2(NC(=O)c3cc(Cl)cc(Cl)c3)CC2)cn1]

Route for SU20666-0066

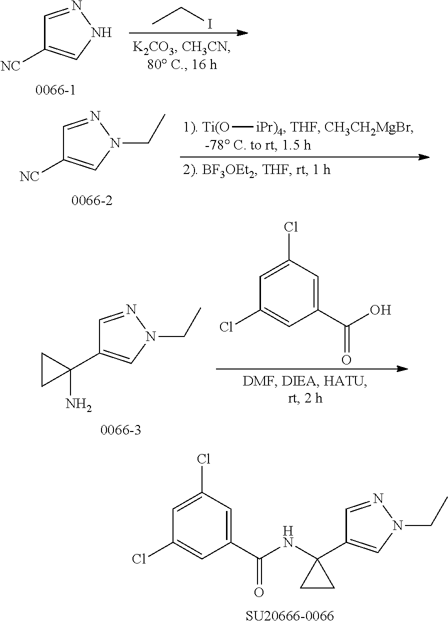

The synthesis of 1-ethyl-1H-pyrazole-4-carbonitrile (0066-2).

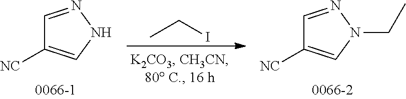

To a solution of compound 0066-1 (2.0 g, 21.5 mmol) in acetonitrile (20 ml) was added iodoethane (4.0 g, 25.8 mmol), K₂CO₃ (3.6 g, 25.8 mmol). The resulting reaction mixture was stirred at 80° C. for 16 h. Then added water, the aqueous phase was extracted with EA, the combined organic phases were dried over anhydrous sodium sulfate, filtered, and concentrated in vacuo, the crude was further purified by C.C. to give the desired product 0066-2 (2.4 g, yield: 92%) as yellow oil.

The synthesis of 1-(1-ethyl-1H-pyrazol-4-yl)cyclopropanamine (0066-3).

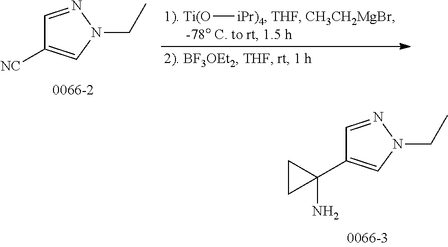

To a solution of compound 0066-2 (0.6 g, 4.9 mmol) in THF (10 ml) was added Ti(O-iPr)₄ (1.7 g, 5.9 mmol) at rt, then the mixture was cooled to −78° C., ethylmagnesium bromide (1.0 M, 12 mL, 12.3 mmol) was added dropwise and stirred at this temperature for 1 h, then warmed to rt and stirred for another 1.5 h. To this reaction mixture, was added BF₃OEt₂ (1.0 M, 9.8 mL, 9.8 mmol) and then stirred at for 16 h at rt. Then the reaction was quenched with water, the aqueous phase was extracted with DCM/MeOH (10/1), the combined organic phases were dried over anhydrous sodium sulfate, filtered, and concentrated in vacuo to give the desired product 0066-3 (600 mg, yield: 80%, purity: 30%) as a white solid, which was used to the next step without further purification.

The synthesis of 3,5-dichloro-N-(1-(1-ethyl-1H-pyrazol-4-yl)cyclopropyl)benzamide (SU20666-0066).

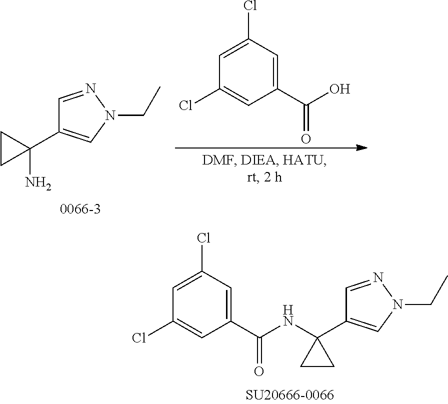

To a solution of compound 0066-3 (333 mg, purity: 30%, 0.66 mmol) in DMF (5 mL) was added 3,5-dichlorobenzoic acid (152 mg, 0.79 mmol), DIEA (170 mg, 1.32 mmol) and HATU (380 mg, 0.98 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0066 (79 mg, yield: 37%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.25%, Rt=1.954 min; MS Calcd.: 323.1; MS Found: 324.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 95.73%, Rt=9.266 min.

¹H NMR (400 MHz, DMSO-d₆) δ 1.02-1.05 (2H, m), 1.11-1.14 (2H, m), 1.31 (3H, t, J=7.2 Hz), 4.02 (2H, q, J=7.2 Hz), 7.22 (1H, s), 7.53 (1H, s), 7.80 (1H, t, J=2.0 Hz), 7.86 (2H, d, J=2.0 Hz), 9.27 (1H, s).

SU20666-0067

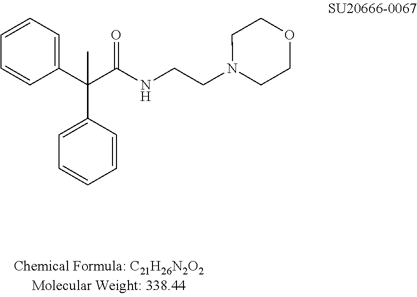 [SMILES: CC(C(=O)NCCN1CCOCC1)(c1ccccc1)c1ccccc1]

Route for SU20666-0067

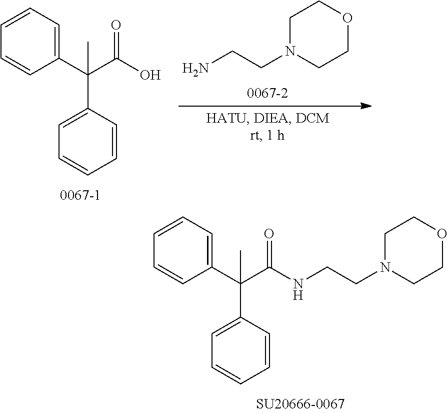 [SMILES: CC(C(=O)O)(c1ccccc1)c1ccccc1 ; NCCN1CCOCC1 ; CC(C(=O)NCCN1CCOCC1)(c1ccccc1)c1ccccc1]

The synthesis of N-(2-morpholinoethyl)-2,2-diphenylpropanamide (SU20666-0067).

To a solution of compound 0067-1 (200 mg, 0.88 mmol) in DCM (5 mL) was added 0067-2 (140 mg, 1.06 mmol), DIEA (343 mg, 2.66 mmol) and HATU (504 mg, 1.33 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0067 (188 mg, yield: 63%) as a white solid.

LC-MS (Agilent LCMS 1200-6120, Column: Waters X-Bridge C18 (50 mm*4.6 mm*3.5μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.936 min; MS Calcd.: 338.2; MS Found: 339.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.71%, Rt=9.365 min.

¹H NMR (400 MHz, DMSO-d₆) δ 1.85 (3H, s), 2.29-2.35 (6H, m), 3.21 (2H, q, J=6.0 Hz), 3.48 (4H, t, J=4.4 Hz), 6.98 (1H, m), 7.19-7.26 (6H, m), 7.29-7.33 (4H, m).

SU20666-0068

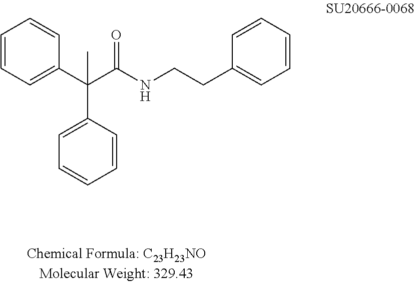 [SMILES: CC(C(=O)NCCc1ccccc1)(c1ccccc1)c1ccccc1]

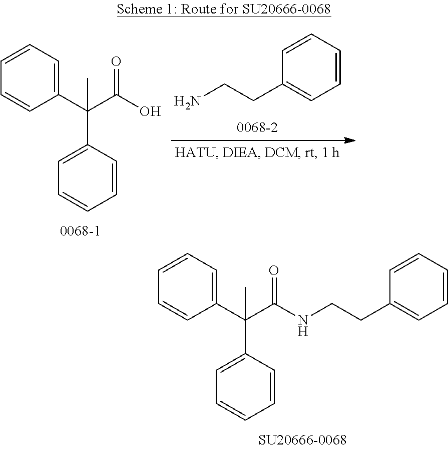 [SMILES: CC(C(=O)O)(c1ccccc1)c1ccccc1 ; NCCc1ccccc1 ; CC(C(=O)NCCc1ccccc1)(c1ccccc1)c1ccccc1]

The synthesis of N-phenethyl-2,2-diphenylpropanamide (SU20666-0068).

To a stirred solution of compound 0068-1 (100 mg, 0.44 mmol) in DCM (5 ml) was added 0068-2 (80 mg, 0.66 mmol), DIEA (170 mg, 1.32 mmol) and HATU (334 mg, 0.88 mmol). The resulting reaction mixture was stirred at rt for 1 h. Then added water, the aqueous phase was extracted with dichloromethane, the combined organic phases were dried over anhydrous sodium sulfate, filtered, and concentrated in vacuopurified by prep-HPLC to give the desired product SU20666-0068 (20 mg, yield: 15.9%) as white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 94.21%, Rt=2.062 min; MS Calcd.: 329.2; MS Found: 330.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 95.96%, Rt=11.517 min.

¹H NMR (400 MHz, DMSO-d₆) δ 1.81 (3H, s), 2.72 (2H, t, J=7.6 Hz), 3.32 (1H, s), 3.36 (1H, d, J=6.8 Hz), 7.09-7.14 (6H, m), 7.20-7.30 (10H, m).

SU20666-0069

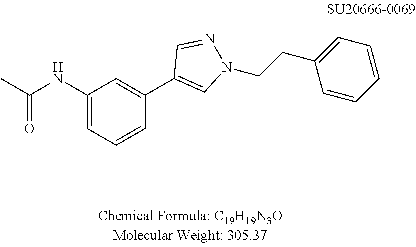 [SMILES: CC(=O)Nc1cccc(-c2cnn(CCc3ccccc3)c2)c1]

Route for SU20666-0069

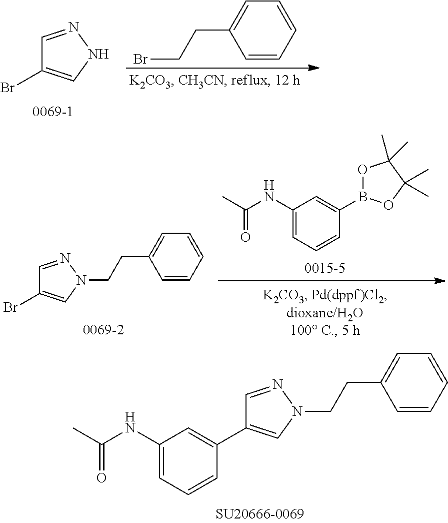 [SMILES: Brc1cn[nH]c1 ; BrCCc1ccccc1 ; Brc1cnn(CCc2ccccc2)c1 ; CC(=O)Nc1cccc(B2OC(C)(C)C(C)(C)O2)c1 ; CC(=O)Nc1cccc(-c2cnn(CCc3ccccc3)c2)c1]

The synthesis of 4-bromo-1-phenethyl-1H-pyrazole (0069-2).

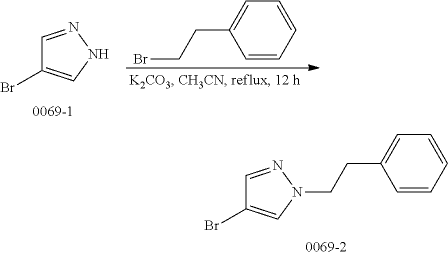 [SMILES: Brc1cn[nH]c1 ; BrCCc1ccccc1 ; Brc1cnn(CCc2ccccc2)c1]

To a solution of compound 0069-1 (1.0 g, 6.8 mmol) in acetonitrile (15 ml) was added (2-bromoethyl)benzene (1.5 g, 8.2 mmol), K₂CO₃ (1.1 g, 8.2 mmol). The resulting reaction mixture was stirred at 90° C. for 12 h. Then added water, the aqueous phase was extracted with EA, the combined organic phases were dried over anhydrous sodium sulfate, filtered, and concentrated in vacuo, the crude was further purified by C.C. to give the desired product 0069-2 (1.2 g, yield: 70%) as yellow oil.

The synthesis of N-(2-morpholinoethyl)-2,2-diphenylpropanamide (SU20666-0069).

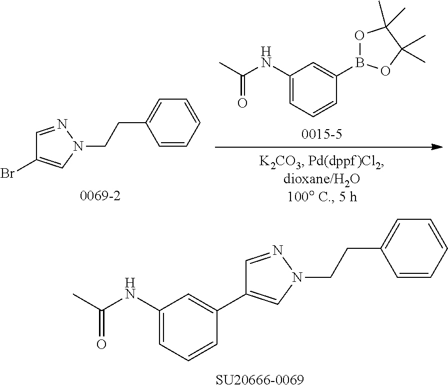 [SMILES: Brc1cnn(CCc2ccccc2)c1 ; CC(=O)Nc1cccc(B2OC(C)(C)C(C)(C)O2)c1 ; CC(=O)Nc1cccc(-c2cnn(CCc3ccccc3)c2)c1]

To a stirred solution of compound 0069-2 (200 mg, 0.80 mmol) in dioxane/water (10 mL/2 mL) was added 0015-5 (250 mg, 0.96 mmol), K₂CO₃ (220 mg, 1.60 mmol), Pd(dppf)Cl₂ (20 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0069 (101 mg, yield: 41%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.83%, Rt=1.753 min; MS Calcd.: 305.1; MS Found: 306.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.690 min.

¹H NMR (400 MHz, DMSO-d₆) δ 2.05 (3H, s), 3.14 (2H, t, J=7.2 Hz), 4.37 (2H, t, J=7.2 Hz), 7.18-7.29 (7H, m), 7.37 (1H, d, J=8.0 Hz), 7.72 (1H, s), 7.78 (1H, s), 7.98 (1H, s), 9.92 (1H, s).

SU20666-0070

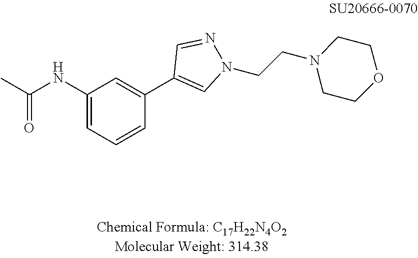 [SMILES: CC(=O)Nc1cccc(-c2cnn(CCN3CCOCC3)c2)c1]

Route for SU20666-0070

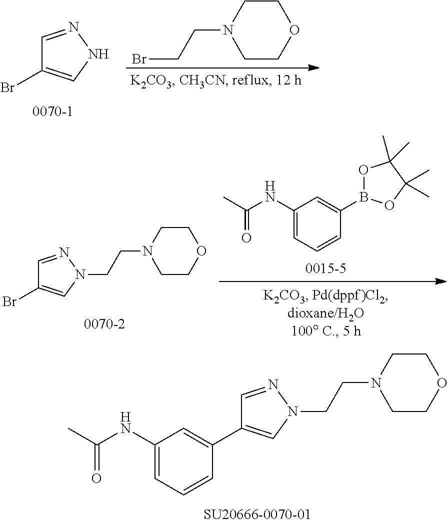

The synthesis of 4-(2-(4-bromo-1H-pyrazol-1-yl)ethyl)morpholine (0070-2).

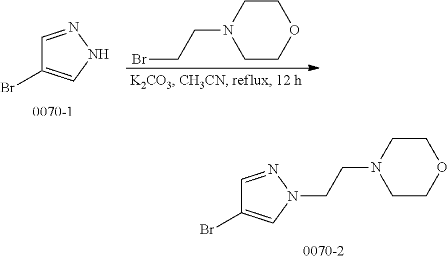 [SMILES: Brc1cn[nH]c1 ; BrCCN1CCOCC1 ; Brc1cnn(CCN2CCOCC2)c1]

To a solution of compound 0070-1 (445 mg, 3.0 mmol) in acetonitrile (15 ml) was added 4-(2-bromoethyl)morpholine (1.0 g, 3.6 mmol), K₂CO₃ (0.50 g, 3.6 mmol). The resulting reaction mixture was stirred at 90° C. for 12 h. Then added water, the aqueous phase was extracted with EA, the combined organic phases were dried over anhydrous sodium sulfate, filtered, and concentrated in vacuo, the crude was further purified by C.C. to give the desired product 0070-2 (0.50 g, yield: 64%) as yellow oil.

The synthesis of N-(2-morpholinoethyl)-2,2-diphenylpropanamide (SU20666-0070).

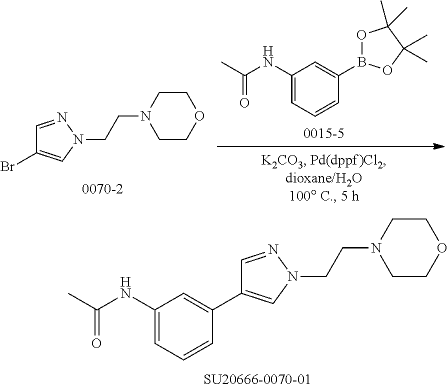 [SMILES: Brc1cnn(CCN2CCOCC2)c1 ; CC(=O)Nc1cccc(B2OC(C)(C)C(C)(C)O2)c1 ; CC(=O)Nc1cccc(-c2cnn(CCN3CCOCC3)c2)c1]

To a stirred solution of compound 0070-2 (200 mg, 0.77 mmol) in dioxane/water (10 mL/2 mL) was added 0015-5 (241 mg, 0.92 mmol), K₂CO₃ (212 mg, 1.64 mmol), Pd(dppf)Cl₂ (20 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0070 (18 mg, yield: 7.5%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.383 min; MS Calcd.: 314.2; MS Found: 315.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=6.276 min.

¹H NMR (400 MHz, DMSO-d₆) δ 2.05 (3H, s), 2.40-2.43 (4H, m), 2.73 (2H, t, J=6.8 Hz), 3.54-3.56 (4H, m), 4.25 (2H, t, J=6.8 Hz), 7.20-7.28 (2H, m), 7.37 (1H, d, J=8.0 Hz), 7.75 (2H, s), 8.09 (1H, s), 9.91 (1H, s).

SU20666-0071

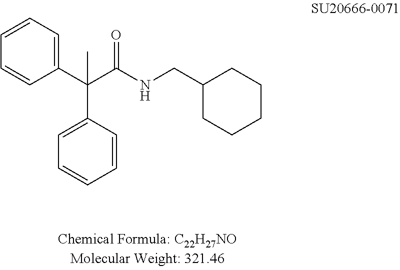 [SMILES: CC(C(=O)NCC1CCCCC1)(c1ccccc1)c1ccccc1]

Route for SU20666-0071

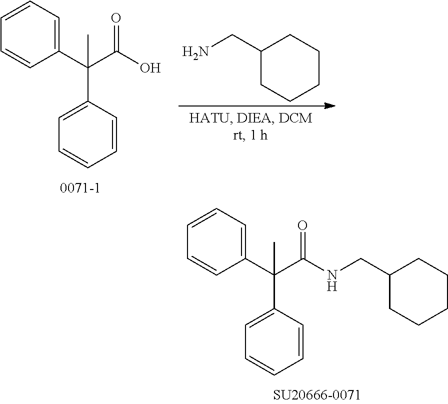 [SMILES: CC(C(=O)O)(c1ccccc1)c1ccccc1 ; NCC1CCCCC1 ; CC(C(=O)NCC1CCCCC1)(c1ccccc1)c1ccccc1]

The synthesis of N-(cyclohexylmethyl)-2,2-diphenylpropanamide (SU20666-0071).

To a solution of compound 0071-1 (200 mg, 0.88 mmol) in DCM (5 mL) was added cyclohexylmethanamine (120 mg, 0.88 mmol), DIEA (343 mg, 2.66 mmol) and HATU (504 mg, 1.33 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0071 (140 mg, yield: 49%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.15%, Rt=2.197 min; MS Calcd.: 321.2; MS Found: 322.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=12.345 min.

¹H NMR (400 MHz, DMSO-d₆) δ 0.73-0.82 (2H, m), 1.05-1.17 (3H, m), 1.39-1.46 (1H, m), 1.52-1.63 (5H, m), 1.85 (3H, s), 2.92 (2H, t, J=6.4 Hz), 7.15-7.24 (7H, m), 7.28-7.32 (4H, m).

SU20666-0072

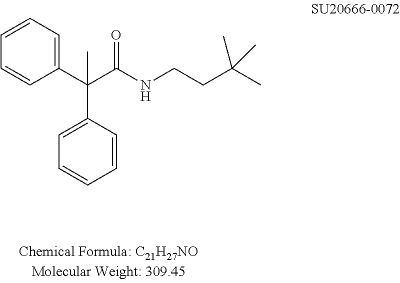 [SMILES: CC(C)(C)CCNC(=O)C(C)(c1ccccc1)c1ccccc1]

Route for SU20666-0072

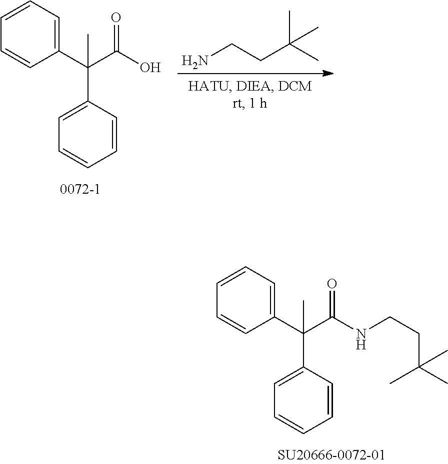

The synthesis of N-(3,3-dimethylbutyl)-2,2-diphenylpropanamide (SU20666-0072).

To a solution of compound 0072-1 (200 mg, 0.88 mmol) in DCM (5 mL) was added 3,3-dimethylbutan-1-amine (107 mg, 1.06 mmol), DIEA (343 mg, 2.66 mmol) and HATU (504 mg, 1.33 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0072 (212 mg, yield: 78%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.368 min; MS Calcd.: 309.2; MS Found: 310.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.988 min.

¹H NMR (400 MHz, DMSO-d₆) δ 0.85 (9H, s), 1.29-1.33 (2H, m), 1.83 (3H, s), 3.07-3.13 (2H, m), 7.14-7.16 (4H, m), 7.21-7.24 (3H, m), 7.28-7.32 (4H, m).

SU20666-0074

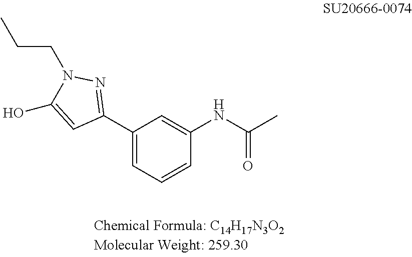 [SMILES: CCCn1nc(-c2cccc(NC(C)=O)c2)cc1O]

Route for SU20666-0074

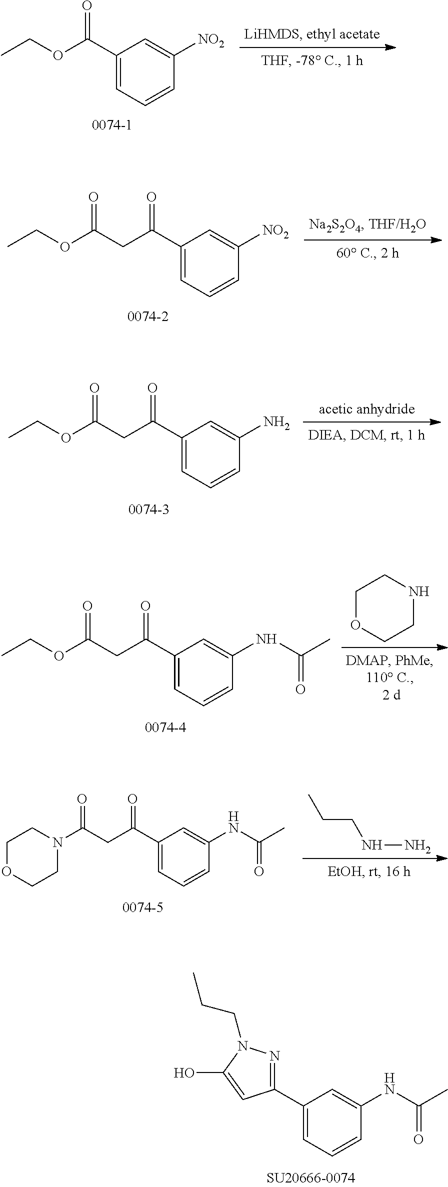

The synthesis of ethyl 3-(3-nitrophenyl)-3-oxopropanoate (0074-2).

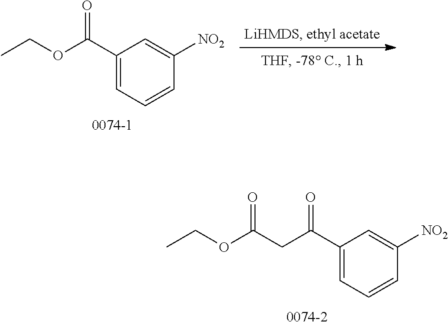 [SMILES: CCOC(=O)c1cccc([N+](=O)[O-])c1 ; CCOC(=O)CC(=O)c1cccc([N+](=O)[O-])c1]

To a solution of compound LiHMDS (1.0 M, 55 mL, 63.5 mmol) in THF (50 mL) under inert atmosphere, was added ethyl acetate (2.7 mL, 27.6 mmol) dropwise at −78° C., after stirring for 0.5 h at this temperature, 0074-1 (5.0 g, 27.6 mmol) was added and stirred for another 1 h at −78° C., then added water, the aqueous phase was extracted with EA, the combined organic phases were dried over anhydrous sodium sulfate, filtered, and concentrated, the crude was purified by C.C. to give the desired product 0074-2 (5.4 g, yield: 82%) as a white solid.

The synthesis of ethyl 3-(3-aminophenyl)-3-oxopropanoate (0074-3).

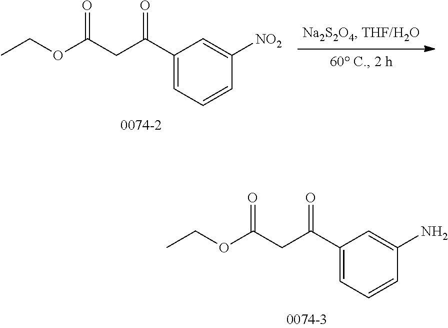 [SMILES: CCOC(=O)CC(=O)c1cccc([N+](=O)[O-])c1 ; CCOC(=O)CC(=O)c1cccc(N)c1]

To a solution of compound 0074-2 (3.0 g, 12.7 mmol) in THF/H₂O (100 mL/50 mL) was added sodium dithionite (22.0 g, 127.0 mmol), the reaction mixture was stirred for 2 h at 60° C., then added water, the aqueous phase was extracted with EA, the combined organic phases were dried over anhydrous sodium sulfate, filtered, and concentrated, the crude was purified by C.C. to give the desired product 0074-3 (1.2 g, yield: 46%) as yellow oil.

The synthesis of ethyl 3-(3-acetamidophenyl)-3-oxopropanoate (0074-4).

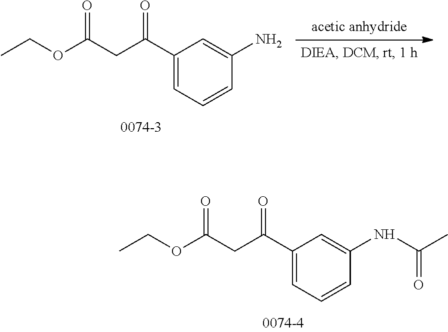 [SMILES: CCOC(=O)CC(=O)c1cccc(N)c1 ; CCOC(=O)CC(=O)c1cccc(NC(C)=O)c1]

To a solution of compound 0074-3 (1.2 g, 5.8 mmol) in DCM (20 mL) was added acetic anhydride (0.88 g, 8.7 mmol) and DIEA (1.50 g, 11.6 mmol), the reaction mixture was stirred for 1 h at rt, then added water, the aqueous phase was extracted with DCM, the combined organic phases were dried over anhydrous sodium sulfate, filtered, and concentrated, the crude was purified by C.C. to give the desired product 0074-4 (0.80 g, yield: 56%) as yellow oil.

The synthesis of N-(3-(3-morpholino-3-oxopropanoyl)phenyl)acetamide (0074-5).

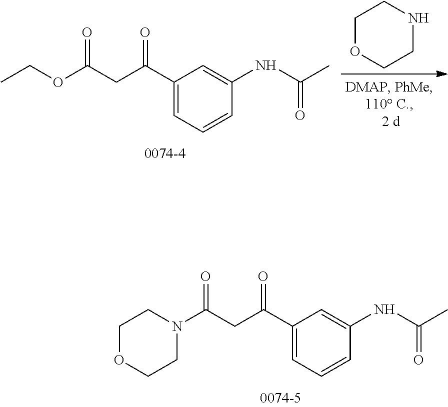

To a solution of compound 0074-4 (0.80 g, 3.2 mmol) in toluene (5 mL) was added morpholine (0.84 g, 9.6 mmol) and DMAP (0.12 g, 0.96 mmol), the reaction mixture was stirred for 48 h at 110° C., then added water, the aqueous phase was extracted with EA, the combined organic phases were dried over anhydrous sodium sulfate, filtered, and concentrated, the crude was purified by C.C. to give the desired product 0074-5 (0.40 g, yield: 43%) as a brown solid.

The synthesis of N-(3-(5-hydroxy-1-propyl-1H-pyrazol-3-yl)phenyl)acetamide (SU20666-0074).

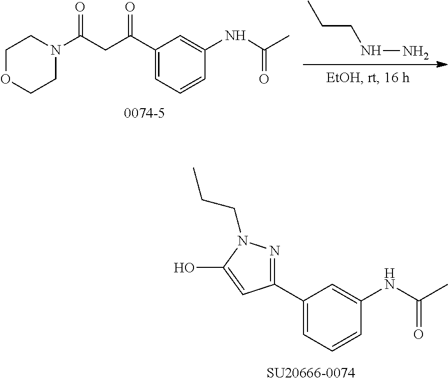

To a solution of compound 0074-5 (100 mg, 0.34 mmol) in EtOH (5 mL) was added propylhydrazine (77 mg, 0.69 mmol), the reaction mixture was stirred for 16 h at rt, then concentrated, the crude was purified by prep-HPLC to give the desired product SU20666-0074 (1.2 mg, yield: 1.3%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.313 min; MS Calcd.: 259.1; MS Found: 260.3 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 99.02%, Rt=5.920 min.

¹H NMR (400 MHz, DMSO-d₆) δ 0.86 (3H, t, J=7.2 Hz), 1.72 (2H, q, J=7.2 Hz), 2.03 (3H, s), 3.83 (2H, t, J=6.4 Hz), 5.65 (1H, s), 7.24 (1H, t, J=7.6 Hz), 7.31 (1H, d, J=7.6 Hz), 7.54 (1H, d, J=8.0 Hz), 7.87 (1H, s), 9.92 (1H, s).

SU20666-0075

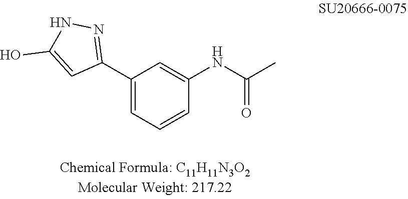 [SMILES: CC(=O)Nc1cccc(-c2cc(O)[nH]n2)c1]

Route for SU20666-0075

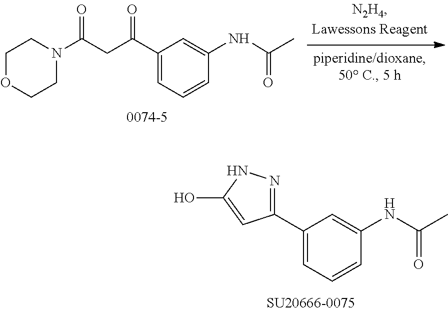 [SMILES: CC(=O)Nc1cccc(C(=O)CC(=O)N2CCOCC2)c1 ; CC(=O)Nc1cccc(-c2cc(O)[nH]n2)c1]

The synthesis of N-(3-(5-hydroxy-1H-pyrazol-3-yl)phenypacetamide (SU20666-0075).

To a solution of compound 0074-5 (300 mg, 1.0 mmol) in piperidine/dioxane (1/19, 10 mL) was added N₂H₄ (62 mg, 1.2 mmol) and Lawessons Reagent (460 mg, 1.1 mmol), the reaction mixture was heated to 50° C. and stirred for 5 h, then added water, the aqueous phase was extracted with EA, the combined organic phases were dried over anhydrous sodium sulfate, filtered, and concentrated, the crude was purified by prep-HPLC to give the desired product SU20666-0075 (42 mg, yield: 19%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 90.29%, Rt=1.135 min; MS Calcd.: 217.1; MS Found: 218.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN +0.1% TFA] in 10 min, then under this condition for 5 min, finally changed to 95% [water+0.1% TFA] and 5% [CH₃CN+0.1% TFA] in 0.1 min and under this condition for 5 min), Purity: 97.10%, Rt=4.912 min.

¹H NMR (400 MHz, DMSO-d₆) δ 2.05 (3H, s), 5.73 (1H, s), 7.31-7.34 (2H, m), 7.50-7.51 (1H, m), 7.81 (1H, s), 9.98 (1H, s).

SU20666-0077

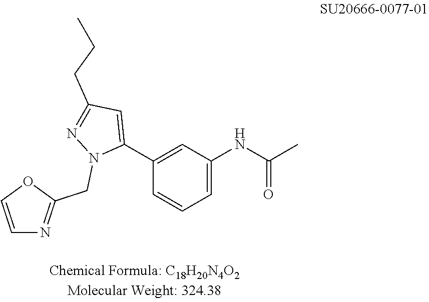 [SMILES: CCCc1cc(-c2cccc(NC(C)=O)c2)n(Cc2ncco2)n1]

Route for SU20666-0077

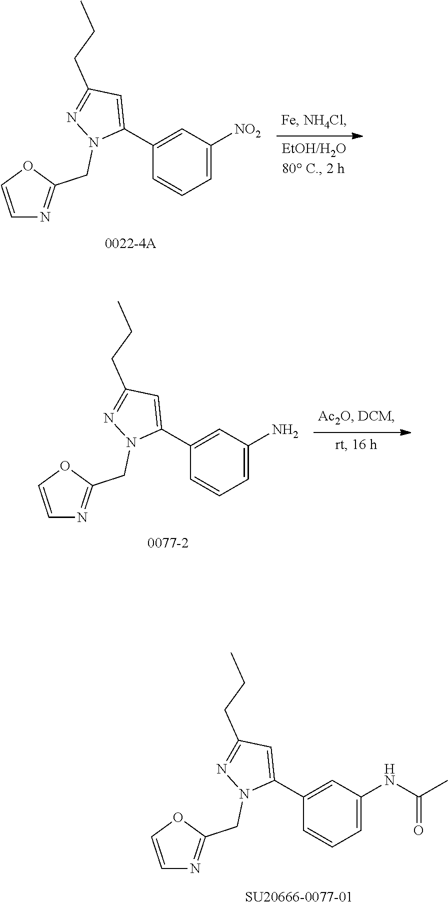 [SMILES: CCCc1cc(-c2cccc([N+](=O)[O-])c2)n(Cc2ncco2)n1 ; CCCc1cc(-c2cccc(N)c2)n(Cc2ncco2)n1 ; CCCc1cc(-c2cccc(NC(C)=O)c2)n(Cc2ncco2)n1]

The synthesis of 3-(1-(oxazol-2-ylmethyl)-3-propyl-1H-pyrazol—S—yl)aniline (0077-2).

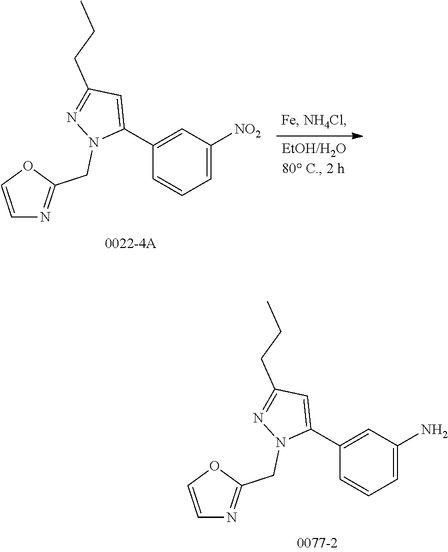 [SMILES: CCCc1cc(-c2cccc([N+](=O)[O-])c2)n(Cc2ncco2)n1 ; CCCc1cc(-c2cccc(N)c2)n(Cc2ncco2)n1]

To a stirred solution of 0022-4A (30 mg, 0.096 mmol) in EtOH/H₂O (10 mL/2 mL) was added Fe powder (28 mg, 0.48 mmol) and NH₄Cl (25 mg, 0.48 mmol) at rt. The resulting reaction mixture was stirred for 2 h at 80° C. Then added water, the aqueous phase was extracted with ethyl acetate, the combined organic phases were dried over anhydrous sodium sulfate, filtered, and concentrated in vacuo to give the desired product 0077-2 (22 mg, yield: 81%) as a yellow solid.

The synthesis of N-(3-(1-(oxazol-2-ylmethyl)-3-propyl-1H-pyrazol—S—yl)phenyl)acetamide (SU20666-0077).

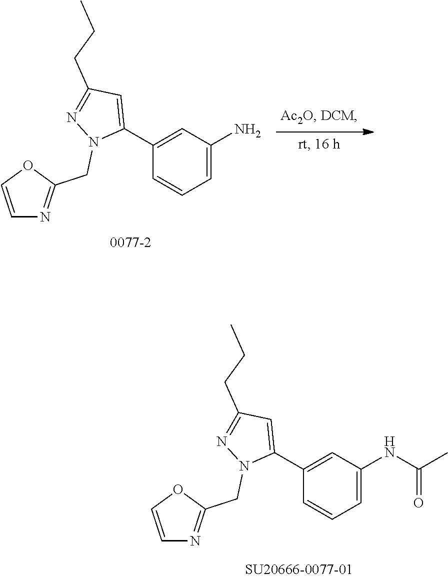 [SMILES: CCCc1cc(-c2cccc(N)c2)n(Cc2ncco2)n1 ; CCCc1cc(-c2cccc(NC(C)=O)c2)n(Cc2ncco2)n1]

To a stirred solution of 0077-2 (22 mg, 0.078 mmol) in DCM (10 mL) was added Ac₂O (16 mg, 0.15 mmol) at rt. The resulting reaction mixture was stirred for 16 h at rt. Then added water, the aqueous phase was extracted with DCM, the combined organic phases were dried over anhydrous sodium sulfate, filtered, and concentrated in vacuo and purified by prep-HPLC to give the desired product SU20666-0077 (10 mg, yield: 40%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 94.96%, Rt=1.757 min; MS Calcd.: 324.1; MS Found: 325.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.805 min.

¹H NMR (400 MHz, DMSO-d₆) δ 0.92 (3H, t, J=6.8 Hz), 1.57-1.63 (2H, m), 2.05 (3H, s), 2.47-2.49 (2H, m), 5.37 (2H, s), 6.21 (1H, s), 7.17 (1H, s), 7.21 (1H, d, J=7.6 Hz), 7.39 (1H, t, J=8.0 Hz), 7.58 (1H, d, J=8.0 Hz), 7.75 (1H, s), 8.05 (1H, s), 10.04(1H, s).

SU20666-0078

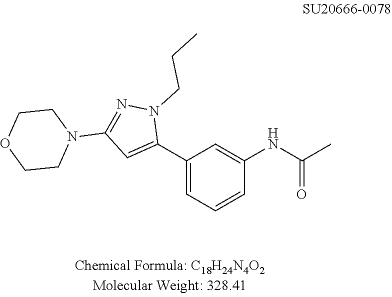 [SMILES: CCCn1nc(N2CCOCC2)cc1-c1cccc(NC(C)=O)c1]

Route for SU20666-0078

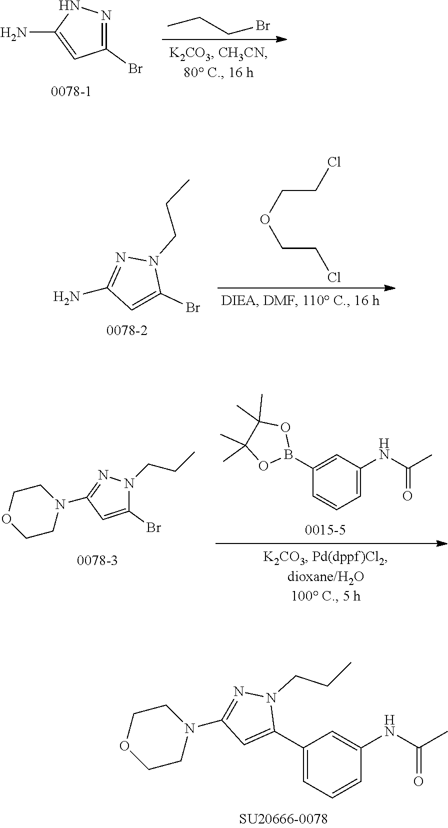

The synthesis of 5-bromo-1-propyl-1H-pyrazol-3-amine (0078-2).

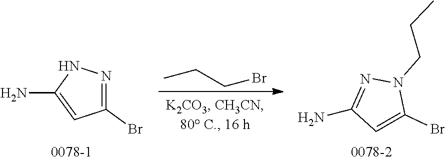

To a solution of compound 0078-1 (2.5 g, 15.4 mmol) in acetonitrile (25 mL) was added 1-bromopropane (2.3 g, 18.5 mmol), K₂CO₃ (2.6 g, 18.5 mmol). The resulting reaction mixture was stirred at 80° C. for 16 h. Then added water, the aqueous phase was extracted with EA, the combined organic phases were dried over anhydrous sodium sulfate, filtered, and concentrated in vacuo, further purified by C.C. to give the desired product 0078-2 (0.80 g, yield: 48%) as a yellow solid.

The synthesis of 4-(5-bromo-1-propyl-1H-pyrazol-3-yl)morpholine (0078-3).

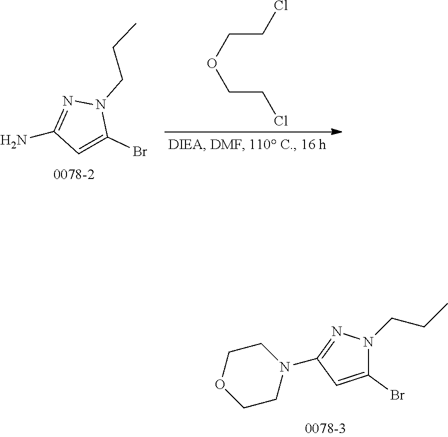

To a solution of compound 0078-2 (500 mg, 2.5 mmol) in DMF (10 mL) was added 1-chloro-2-(2-chloroethoxy)ethane (600 mg, 4.2 mmol), DIEA (645 mg, 5.0 mmol). The resulting reaction mixture was stirred at 110° C. for 16 h. Then added water, the aqueous phase was extracted with EA, the combined organic phases were dried over anhydrous sodium sulfate, filtered, and concentrated in vacuo, further purified by C.C. to give the desired product 0078-3 (270 mg, yield: 40%) as brown liquid.

The synthesis of N-(3-(3-morpholino-1-propyl-1H-pyrazol—S—yl)phenyl)acetamide (SU20666-0078).

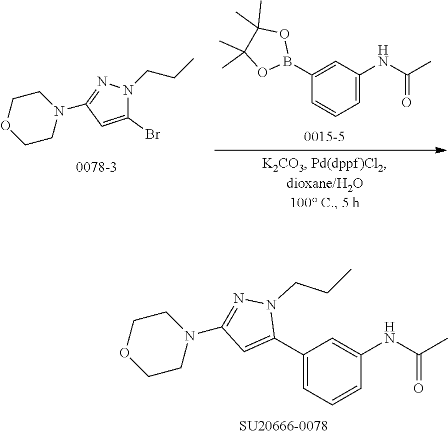

To a stirred solution of compound 0078-3 (130 mg, 0.48 mmol) in dioxane/water (10 mL/2 mL) was added 0015-5 (150 mg, 0.57 mmol), K₂CO₃ (132 mg, 0.96 mmol), Pd(dppf)Cl₂ (20 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0078 (78 mg, yield: 50%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.33%, Rt=1.715 min; MS Calcd.: 328.2; MS Found: 329.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.720 min.

¹H NMR (400 MHz, DMSO-d₆) δ 0.74 (3H, t, J=7.6 Hz), 1.66-1.71 (2H, m), 2.06 (3H, s), 3.07-3.09 (4H, m), 3.68-3.70 (4H, m), 3.86 (2H, t, J=7.6 Hz), 5.82 (1H, s), 7.07 (1H, d, J=7.6 Hz), 7.39 (1H, t, J=7.6 Hz), 7.55 (1H, d, J=8.4 Hz), 7.73 (1H, s), 10.07 (1H, s).

SU20666-0083 and SU20666-0118

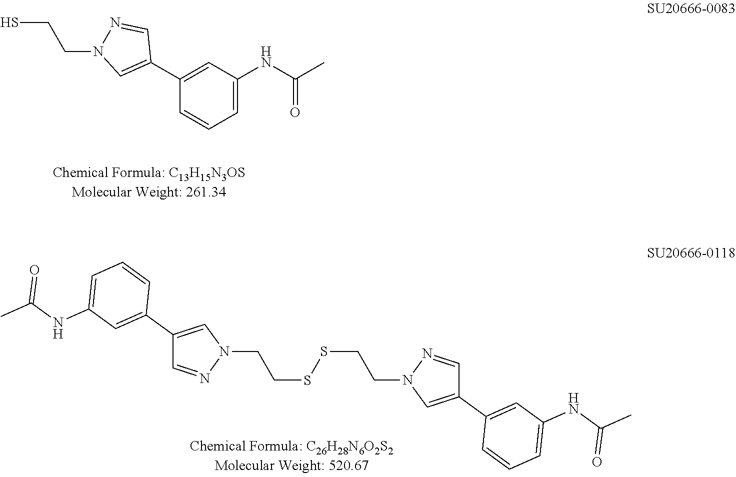 [SMILES: CC(=O)Nc1cccc(-c2cnn(CCS)c2)c1 ; CC(=O)Nc1cccc(-c2cnn(CCSSCCn3cc(-c4cccc(NC(C)=O)c4)cn3)c2)c1]

Route for SU20666-0083 and SU20666-0118

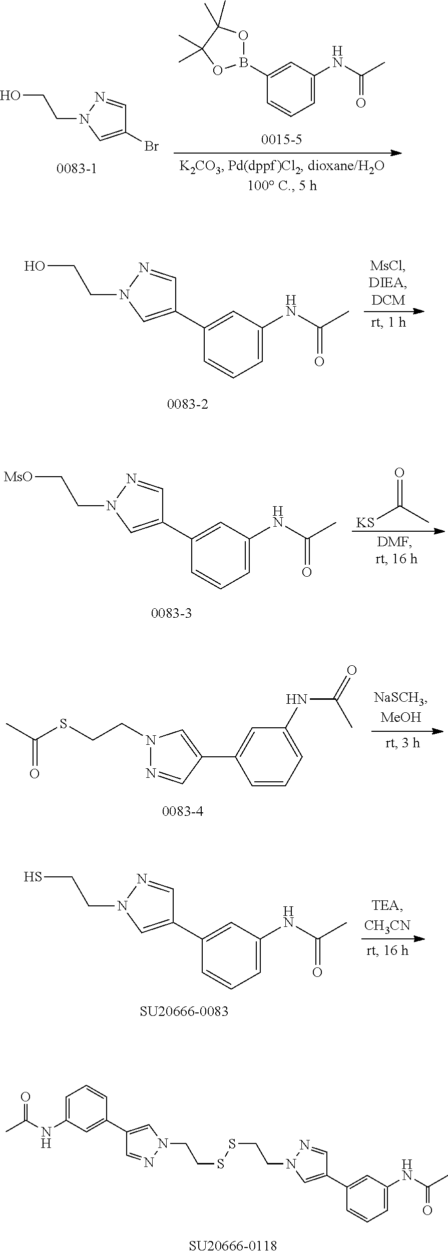

The synthesis of N-(3-(1-(2-hydroxyethyl)-1H-pyrazol-4-yl)phenypacetamide (0083-2).

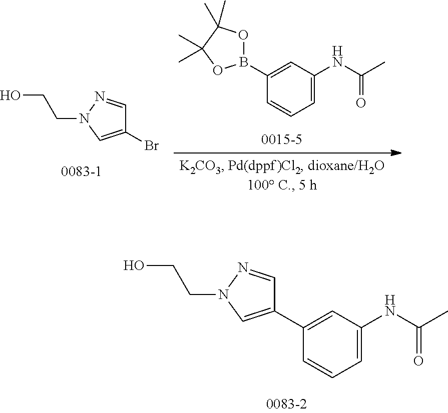 [SMILES: OCCn1cc(Br)cn1 ; CC(=O)Nc1cccc(B2OC(C)(C)C(C)(C)O2)c1 ; CC(=O)Nc1cccc(-c2cnn(CCO)c2)c1]

To a stirred solution of compound 0083-1 (500 mg, 2.6 mmol) in dioxane/water (10 mL/2 mL) was added 0015-5 (1.0 g, 3.9 mmol), K₂CO₃ (1.1 g, 7.9 mmol), Pd(dppf)Cl₂(100 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 0083-2 (230 mg, yield: 36%) as a brown solid.

The synthesis of 2-(4-(3-acetamidophenyl)-1H-pyrazol-1-yl)ethyl methanesulfonate (0083-3).

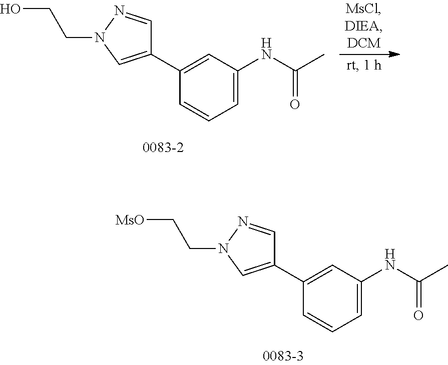 [SMILES: CC(=O)Nc1cccc(-c2cnn(CCO)c2)c1 ; CC(=O)Nc1cccc(-c2cnn(CCOS(C)(=O)=O)c2)c1]

To a stirred solution of 0083-2 (230 mg, 0.94 mmol) in DCM (5 ml) was added DIEA (364 mg, 2.8 mmol) and MsCl (161 mg, 1.4 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo to give the desired product 0083-3 (200 mg, yield: 66%) as a brown solid.

The synthesis of S-(2-(4-(3-acetamidophenyl)-1H-pyrazol-1-yl)ethyl) ethanethioate (0083-4).

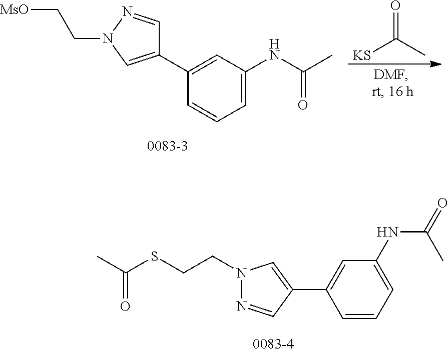

To a stirred solution of compound 0083-3 (200 mg, 0.62 mmol) in DME (5 mL) was added potassium ethanethioate (106 mg, 0.93 mmol). The resulting reaction mixture was stirred for 16 h at rt and then added water, the aqueous phase was extracted with EA, the combined organic phases were dried over anhydrous sodium sulfate, filtered, and concentrated, the crude was purified by C.C. to give the desired product 0083-4 (100 mg, yield: 54%) as a grey solid.

The synthesis of S-(2-(4-(3-acetamidophenyl)-1H-pyrazol-1-yl)ethyl) ethanethioate (SU20666-0083).

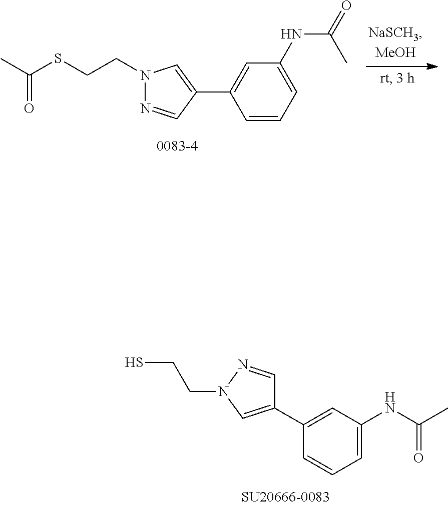 [SMILES: CC(=O)Nc1cccc(-c2cnn(CCSC(C)=O)c2)c1 ; CC(=O)Nc1cccc(-c2cnn(CCS)c2)c1]

To a stirred solution of compound 0083-4 (20 mg, 0.066 mmol) in methanol (1 mL) was added sodium methanethiolate (7 mg, 0.10 mmol). The resulting reaction mixture was stirred for 3 h at rt and then added water, the aqueous phase was extracted with EA, the combined organic phases were dried over anhydrous sodium sulfate, filtered, and concentrated, the crude was purified by prep-HPLC to give the desired product SU20666-0083 (2 mg, yield: 12%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 94.26%, Rt=1.441 min; MS Calcd.: 261.1; MS Found: 262.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 95.91%, Rt=6.998 min.

¹H NMR (400 MHz, DMSO-d₆) δ 2.05 (3H, s), 2.38 (1H, t, J=8.4 Hz), 2.94 (2H, q, J=7.2 Hz), 4.28 (2H, t, J=7.2 Hz), 7.22-7.29 (2H, m), 7.48 (1H, d, J=7.6 Hz), 7.75-7.79 (2H, m), 8.11 (1H, s), 9.92 (1H, s).

The synthesis of N,N′-((1,1′-(disulfanediylbis(ethane-2,1-diyl))bis(1H-pyrazole-4,1-diyl))bis(3,1-phenylene))diacetamide (SU20666-0118).

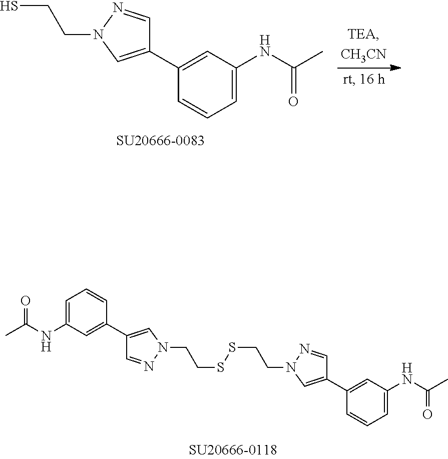

To a stirred solution of compound SU20666-0083 (70 mg, 0.27 mmol) in acetonitrile (3 mL) was added TEA (130 mg, 1.3 mmol). The resulting reaction mixture was stirred for 16 h at rt and then added water, the aqueous phase was extracted with EA, the combined organic phases were dried over anhydrous sodium sulfate, filtered, and concentrated, the crude was purified by prep-HPLC to give the desired product SU20666-0118 (16 mg, yield: 11%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.50%, Rt=1.661 min; MS Calcd.: 520.2; MS Found: 521.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 95.91%, Rt=8.008 min.

¹H NMR (400 MHz, DMSO-d₆) δ 2.04 (6H, s), 3.23 (4H, t, J=6.4 Hz), 4.42 (4H, t, J=6.4 Hz), 7.23-7.26 (4H, m), 7.38 (2H, d, J=7.6 Hz), 7.75-7.79 (4H, m), 8.12 (2H, s), 9.92 (2H, s).

The names SU20666-0085, SU20666-0085-01, SP 85, and 85 all refer to the same compound having the formula:

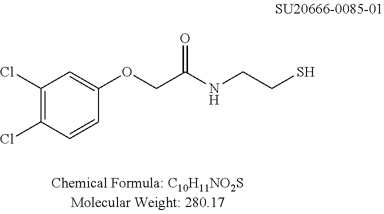 [SMILES: O=C(COc1ccc(Cl)c(Cl)c1)NCCS]

Route for SU20666-0085

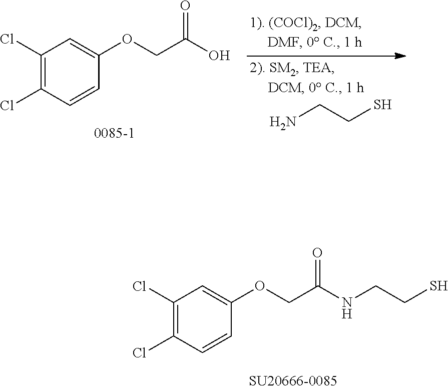

The synthesis of 2-(3,4-dichlorophenoxy)-N-(2-mercaptoethyl)acetamide (SU20666-0085).

To a stirred solution of 2-(3,4-dichlorophenoxy)acetic acid (0085-1, 500 mg, 2.3 mmol) in dichloromethane (30 ml) was added oxalyl chloride (1.4 g, 11.4 mmol) and DMF (0.1 mL) at 0° C. The resulting reaction mixture was stirred at 0° C. for 1 h and concentrated in vacuo, the crude was dissolved in dichloromethane (30 mL), was added TEA (440 mg, 4.4 mmol) and 2-aminoethanethiol (340 mg, 4.4 mmol) at 0° C., then the reaction mixture was stirred for another 1 h at 0° C. Water (20 mL) was added, the aqueous phase was extracted with dichloromethane, the combined organic phases were dried over anhydrous sodium sulfate, filtered, and concentrated in vacuo, purified by prep-HPLC to give the desired product 2-(3,4-dichlorophenoxy)-N-(2-mercaptoethyl)acetamide (130 mg, yield: 22%) as a yellow solid.

LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min.), Purity: 98.70%, Rₜ=0.720 min; MS Calcd.: 279.0; MS Found: 280.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 97.40%, Rt=9.280 min.

¹H NMR (400 MHz, CDCl₃) δ 1.29 (1H, t, J=8.8 Hz), 2.62-2.67(2H, m), 3.48 (2H, q, J=6.4 Hz), 4.41 (2H, s), 6.73 (1H, dd, J=3.2, 9.2 Hz), 6.82 (1H, brs.), 6.99 (1H, d, J=2.8 Hz), 7.31 (1H, d, J=8.8 Hz).

The names SU20666-0087, SU20666-0087-01, SP 87, and 87 all refer to the same compound having the formula:

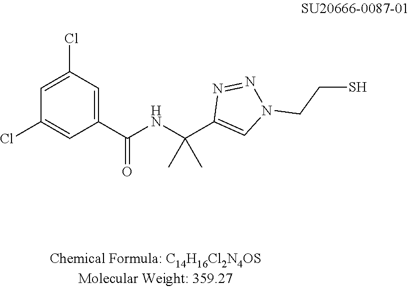 [SMILES: CC(C)(NC(=O)c1cc(Cl)cc(Cl)c1)c1cn(CCS)nn1]

Route for SU20666-0087

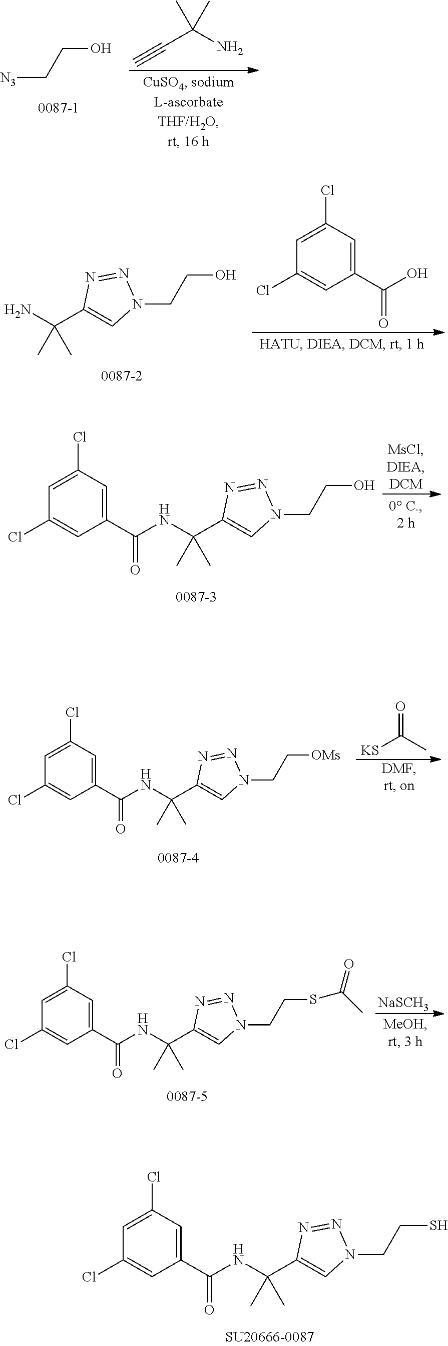

The synthesis of 2-(4-(2-aminopropan-2-yl)-1H-1,2,3-triazol-1-yl)ethanol (0087-2).

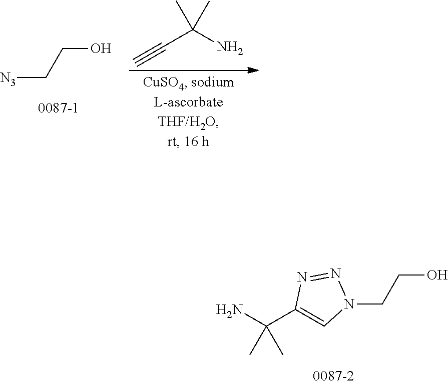

To a stirred solution of compound 0087-1 (1 g, 11.5 mmol) in THF/water (30 ml/6 ml) was added 2-methylbut-3-yn-2-amine (954 mg, 11.5 mmol), CuSO₄ (1.44 g, 5.75 mmol) and sodium L-ascorbate (1.14 g, 5.75 mmol). The resulting reaction mixture was stirred at rt for 16 h. Then removed solvent, added water, the aqueous phase was extracted with dichloromethane, the combined organic phases were dried over anhydrous sodium sulfate, filtered, and concentrated in vacuo to give the desired product 0087-2 (1.9 g, yield: 97.2%) as a green solid.

The synthesis of 3,5-dichloro-N-(2-(1-(2-hydroxyethyl)-1H-1,2,3-triazol-4-yl)propan-2-yl)benzamide (0087-3).

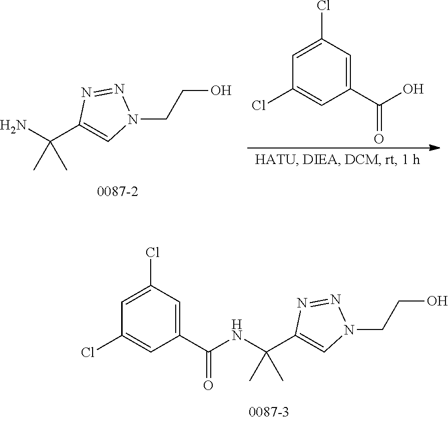 [SMILES: CC(C)(N)c1cn(CCO)nn1 ; O=C(O)c1cc(Cl)cc(Cl)c1 ; CC(C)(NC(=O)c1cc(Cl)cc(Cl)c1)c1cn(CCO)nn1]

To a stirred solution of compound 0087-2 (1.9 g, 11.2 mmol) in DCM (20 ml) was added 3,5-dichlorobenzoic acid (1.4 g, 7.45 mmol), DIEA (2.9 g, 22.4 mmol) and HATU (4.0 g, 11.2 mmol). The resulting reaction mixture was stirred at rt for 1 h. Then added water, the aqueous phase was extracted with dichloromethane, the combined organic phases were dried over anhydrous sodium sulfate, filtered, and concentrated in vacuopurified by prep-HPLC to give the desired product 0087-3 (1.5 g, yield: 60%) as white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 88.81%, Rt=1.700 min; MS Calcd.: 342.0; MS Found:343.2 [M+H]⁺.

The synthesis of 2-(4-(2-(3,5-dichlorobenzamido)propan-2-yl)-1H-1,2,3-triazol-1-yl)ethyl methanesulfonate (0087-4).

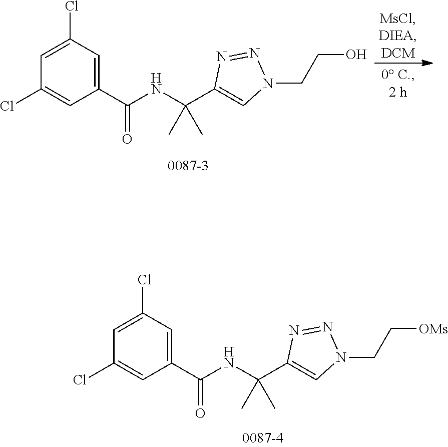 [SMILES: CC(C)(NC(=O)c1cc(Cl)cc(Cl)c1)c1cn(CCO)nn1 ; CC(C)(NC(=O)c1cc(Cl)cc(Cl)c1)c1cn(CCOS(C)(=O)=O)nn1]

To a stirred solution of compound 0087-3 (1.5 g, 4.37 mmol) in DCM (15 ml) was added MsCl (0.75 g, 6.56 mmol) and DIEA (1.7 g, 13 mmol) under ice-water. The resulting reaction mixture was stirred at O° C. for 1 h. Then added water, the aqueous phase was extracted with dichloromethane, the combined organic phases were dried over anhydrous sodium sulfate, filtered, and concentrated in vacuo to give the desired product 0087-4 (1.0 g, yield: 54.3%) as a yellow solid.

LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min), Purity: 75.85%, Rt=0.685 min; MS Calcd.: 420.0; MS Found:421.2[M+H]⁺.

The synthesis of S-2-(4-(2-(3,5-dichlorobenzamido)propan-2-yl)-1H-1,2,3-triazol-1-yl)ethyl ethanethioate (0087-5).

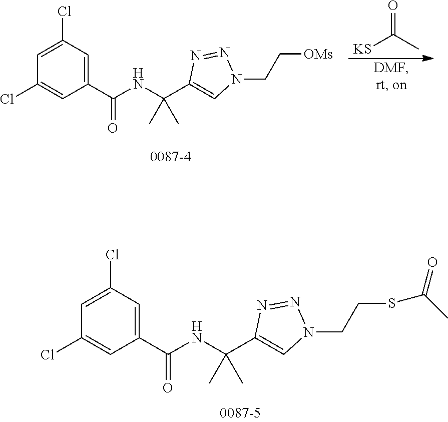

To a stirred solution of compound 0087-4 (1.0 g, 2.4 mmol) in DMF (10 ml) was added potassium ethanethioate (0.32 g, 2.9 mmol). The resulting reaction mixture was stirred at rt overnight. Then added water, the aqueous phase was extracted with EA, the combined organic phases were dried over anhydrous sodium sulfate, filtered, and concentrated in vacuo to give the desired product 0087-5 (150 mg, yield: 15.8%) as yellow solid.

LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min), Purity: 52.77%, Rt=0.740 min; MS Calcd.: 400.0; MS Found: 401.2 [M+H]⁺.

The synthesis of 3,5-dichloro-N-(2-(1-(2-mercaptoethyl)-1H-1,2,3-triazol-4-yl)propan-2-yl)benzamide (SU20666-0087).

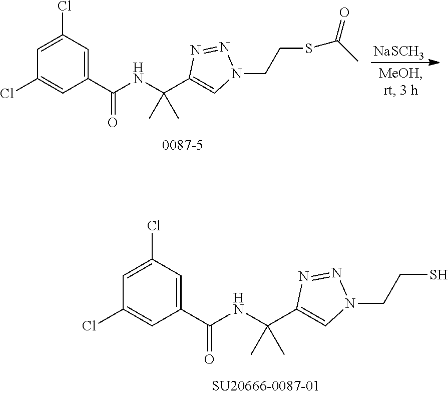

To a stirred solution of compound 0087-5 (150 mg, 0.38 mmol) in MeOH (5 ml) was added NaSCH₃ (41 mg, 0.57 mmol). The resulting reaction mixture was stirred at rt for 3h. Then removed the solvent, added water, the aqueous phase was extracted with DCM, the combined organic phases were dried over anhydrous sodium sulfate, filtered, and concentrated in vacuo, purified by prep-HPLC to give the desired product SU20666-0087 (70 mg, yield: 52.2%) as a brown solid.

LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min.), Purity: 97.94%, Rt=1.770 min; MS Calcd.: 358.0; MS Found:357.8[M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 95.82%, Rt=9.038 min.

¹H NMR (400 MHz, DMSO-d₆) δ 1.70 (6H, s), 2.43 (1H, t, J=8.0 Hz), 2.95 (2H, q, J=7.6 Hz), 4.46 (2H, t, J=7.2 Hz), 7.78-7.84 (3H, m), 7.98 (1H, s), 8.60 (1H, s).

SU-20666-0089

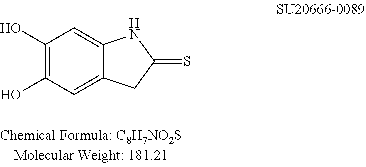 [SMILES: Oc1cc2c(cc1O)NC(=S)C2]

Route for SU20666-0089

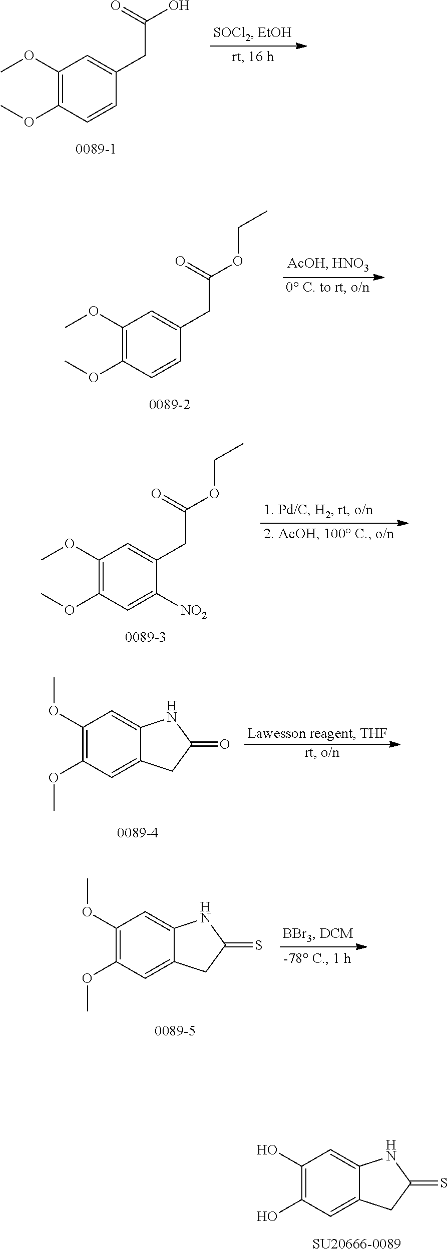 [SMILES: COc1ccc(CC(=O)O)cc1OC ; CCOC(=O)Cc1ccc(OC)c(OC)c1 ; CCOC(=O)Cc1cc(OC)c(OC)cc1[N+](=O)[O-] ; COc1cc2c(cc1OC)NC(=O)C2 ; COc1cc2c(cc1OC)NC(=S)C2 ; Oc1cc2c(cc1O)NC(=S)C2]

The synthesis of ethyl 2-(3,4-dimethoxyphenyl)acetate (0089-2).

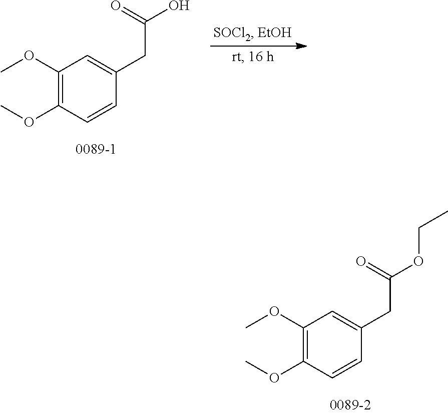 [SMILES: COc1ccc(CC(=O)O)cc1OC ; CCOC(=O)Cc1ccc(OC)c(OC)c1]

To a solution of 0089-1 (5.0 g, 25.5 mmol) in EtOH (30 mL) was added SOCl₂ (3.04 g, 25.5 mmol). The mixture was stirred at rt for 16 h. Then concentrated in vacuo to give 0089-2 (4 g, 70%) as a yellow solid.

The synthesis of ethyl 2-(4,5-dimethoxy-2-nitrophenyl)acetate (0089-3).

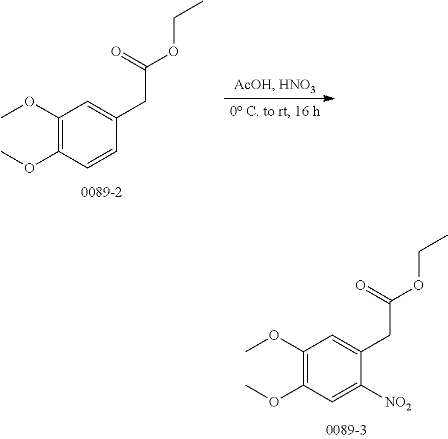 [SMILES: CCOC(=O)Cc1ccc(OC)c(OC)c1 ; CCOC(=O)Cc1cc(OC)c(OC)cc1[N+](=O)[O-]]

To a solution of 0089-2 (4,0 g, 14.9 mmol) in CH₃COOH (15 mL) was added HNO3 (5 mL). The mixture was stirred at 0° C. to rt for 16 h. Then added water, the solid was collected to give compound 0089-3 (2.5 g, 63%) as a yellow solid.

The synthesis of 5,6-dimethoxyindolin-2-one (0089-4).

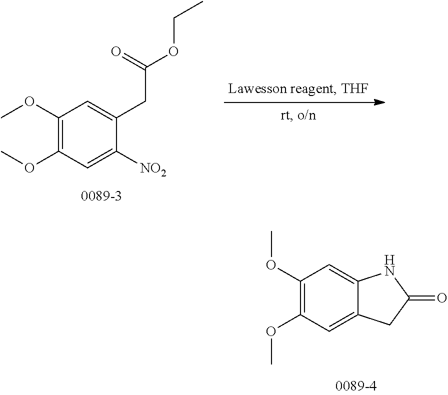 [SMILES: CCOC(=O)Cc1cc(OC)c(OC)cc1[N+](=O)[O-] ; COc1cc2c(cc1OC)NC(=O)C2]

To a solution of 0089-3 (2.5 g, 9.3 mmol) in EtOH (15 mL) was added Pd/C (10%, 250 mg), the mixture was stirred at rt for o/n under H₂ atmosphere (1.0 atm). The mixture was filtered and concentrated in vacuo to give yellow oil. To the oil was added AcOH as solvent (30 mL) and he mixture was stirred at 100° C. for o/n. Then concentrated in vacuo to give crude product, which was purified by pre-HPLC to afford compound 0089-4 (950 mg, 53%) as a yellow solid.

The synthesis of 5,6-dimethoxyindoline-2-thione (0089-5).

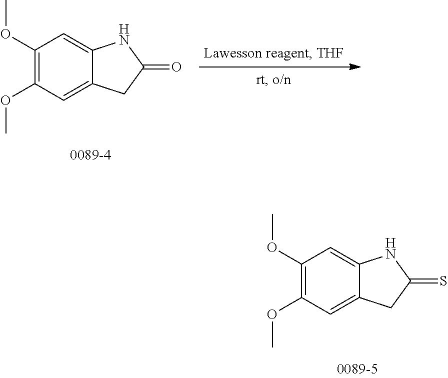

To a solution of 0089-4 (350 mg, 1.8 mmol) in THF (10 mL) was added Lawesson reagent (1.4 g, 3.6 mmol). The mixture was stirred at rt for o/n. Then removed solvent, added water, the aqueous phase was extracted with EA, the combined organic phases were dried over anhydrous sodium sulfate, filtered, and concentrated in vacuo to give the desired product 0089-5 (105 mg, 28%) as a yellow solid.

The synthesis of 5,6-dihydroxyindoline-2-thione (SU20666-0089).

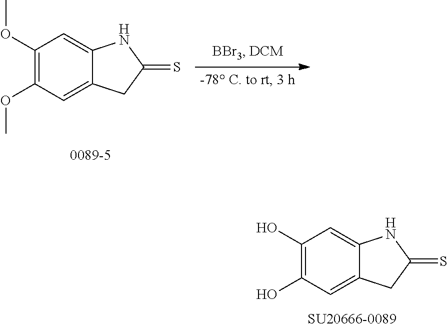

To a solution of 0089-5 (80 mg, 0.38 mmol) in DCM (5 mL) was added BBr₃ (0.5 mL) at −78° C., the mixture was warmed to rt and stirred for 3 h. Water was added, the aqueous phase was extracted with EA, the combined organic phases were dried over anhydrous sodium sulfate, filtered, and concentrated in vacuo, The crude product was purified by pre-HPLC to afford the desired product SU20666-0089 (15 mg, 22%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity is 100%, Rt=1.083 min; MS Calcd.: 181.0; MS Found: 182.2 [M+H]⁺.

¹H NMR (400 MHz, DMSO-d₆) δ 3.82 (s, 2H), 6.46 (s, 1H), 6.68 (s, 1H), 8.68 (s, 1H), 9.05 (s, 1H), 12.26 (s, 1H).

SU20666-0090

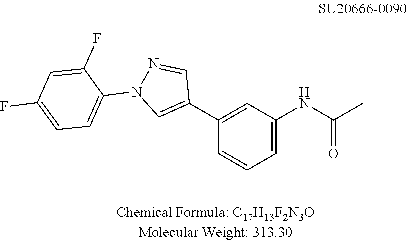 [SMILES: CC(=O)Nc1cccc(-c2cnn(-c3ccc(F)cc3F)c2)c1]

Route for SU20666-0090

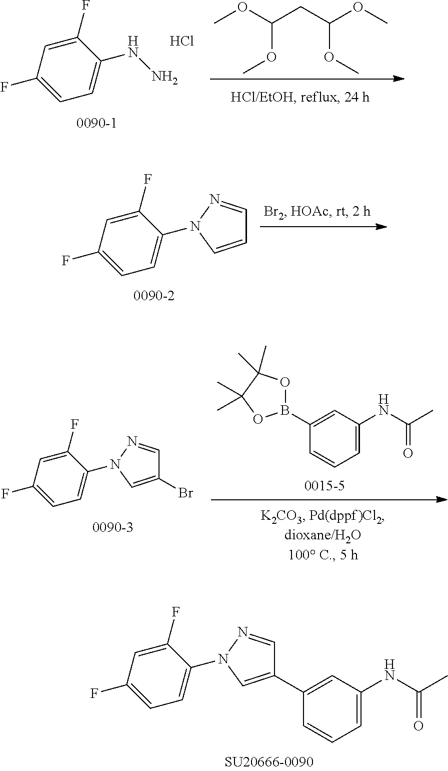

The synthesis of 1-(2,4-difluorophenyl)-1H-pyrazole (0090-2).

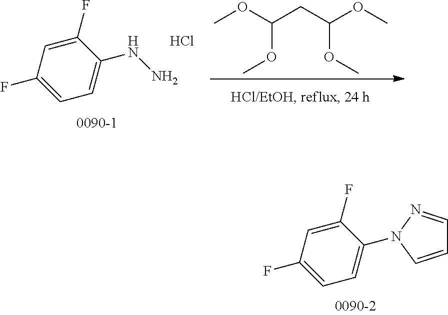

To a stirred solution of compound 0090-1 (3.0 g, 16.7 mmol) in HCl/EtOH (5m1/20ml) was added 1,1,3,3-tetramethoxypropane (4.1 g, 25.0 mmol). The resulting reaction mixture was stirred at 90° C. for 24 h. Then removed the solvent, added water, the aqueous phase was extracted with dichloromethane, the combined organic phases were dried over anhydrous sodium sulfate, filtered, and concentrated in vacuo to give the desired product 0090-2 (2.3 g, yield: 76.7%) as yellow liquid.

LC-MS (Agilent LCMS 1200-6120, Column: Waters X-Bridge C18 (30 mm×4.6 mm×2.7 μm); Column Temperature: 40° C.; Flow Rate: 3.0 mL/min; Mobile Phase: from 95% [water+0.05%TFA] and 5% [CH₃CN+0.05%TFA] to 0% [water+0.05% TFA] and 100% [CH₃CN+0.05%TFA] in 0.8 min, then under this condition for 0.4 min, finally changed to 95% [water+0.05% TFA] and 5% [CH₃CN+0.05%] in 0.01 min), Purity: 94.19%, Rt=0.692 min; MS Calcd.: 180.1; MS Found:181.4[M+H]⁺.

The synthesis of 4-bromo-1-(2,4-difluorophenyl)-1H-pyrazole (0090-3).

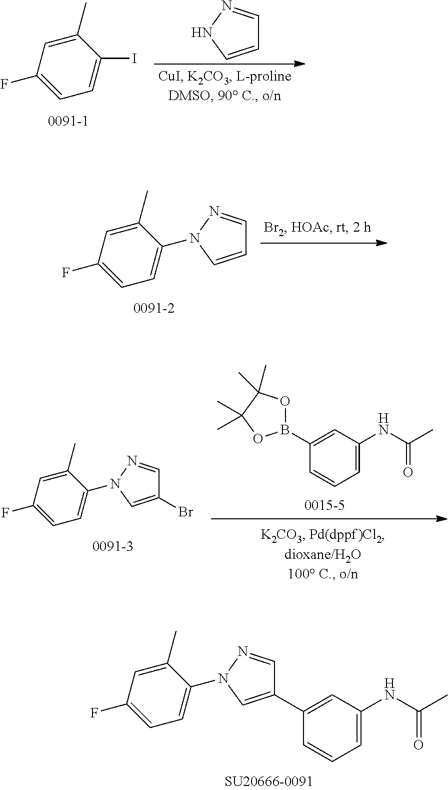

To a stirred solution of compound 0090-2 (500 mg, 2.8 mmol) in HOAc (8ml) was added Br₂ (672 mg, 4.2 mmol). The resulting reaction mixture was stirred at rt overnight. Then added NaHSO₃(aq) and water, the aqueous phase was extracted with EA, the combined organic phases were dried over anhydrous sodium sulfate, filtered, and concentrated in vacuo to give the desired product 0090-3 (340 mg, yield: 78.5%) as yellow liquid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 95.86%, Rt=1.929 min; MS Calcd.: 258.0; MS Found: 259.1 [M+H]⁺.

The synthesis of N-(3-(1-(2,4-difluorophenyl)-1H-pyrazol-4-yl)phenyl)acetamide (SU20666-0090).

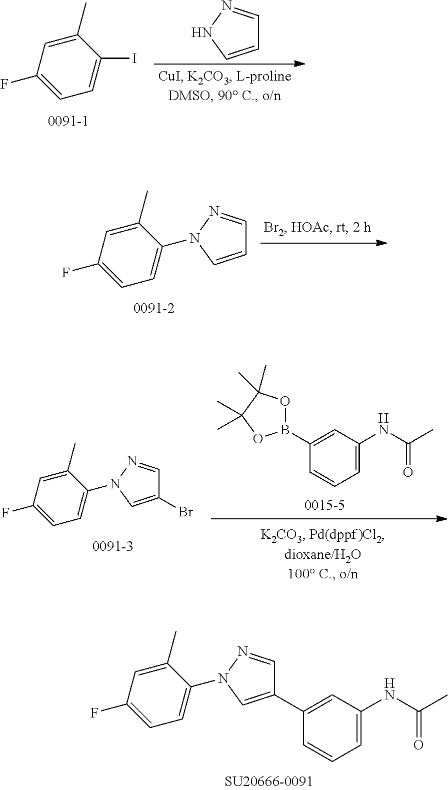

A solution of 0090-3 (300 mg, 1.16 mmol) in dioxane/H₂O (8 ml/2 ml), was added 0015-5 (455 mg, 1.74 mmol), K₂CO₃ (480 mg, 3.48 mmol) and Pd(dppf)Cl₂ (50 mg) under an argon atmosphere. The mixture was stirred at 100° C. for 5 h. After being cooled to room temperature, water was added. The aqueous phase was extracted with DCM (20 mL x 3), and the combined organic phases were dried over anhydrous sodium sulfate, filtered, and concentrated in vacuo. The crude product was purified by prep-HPLC to give compound SU20666-0090 (50 mg, 13.7%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.889 min; MS Calcd.: 313.1; MS Found: 314.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.920 min.

¹H NMR (400 MHz, DMSO-d₆) δ 2.07 (3H, d, J=6.0 Hz), 7.29-7.38 (3H, m), 7.48 (1H, d, J=7.6 Hz), 7.60 (1H, t, J=2.4 Hz), 7.82-7.89 (2H, m), 8.14 (1H, s), 8.52 (1H, d, J=1.6 Hz), 9.98 (1H, s).

SU20666-0091

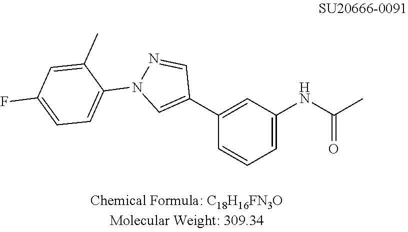 [SMILES: CC(=O)Nc1cccc(-c2cnn(-c3ccc(F)cc3C)c2)c1]

Route for SU20666-0091

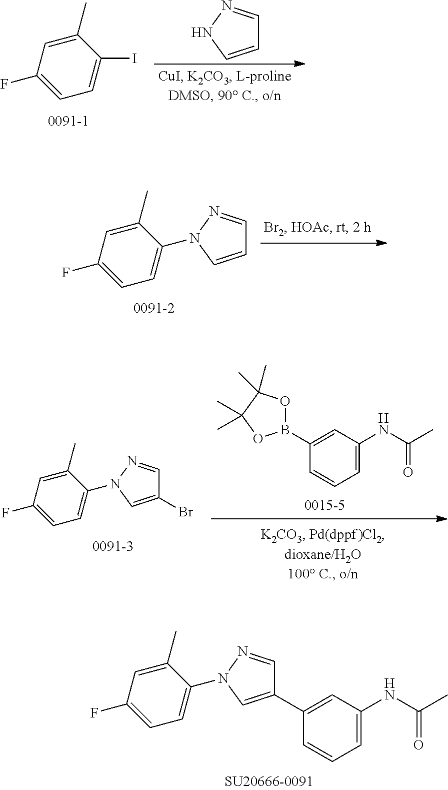

The synthesis of 1-(4-fluoro-2-methylphenyl)-1H-pyrazole (0091-2).

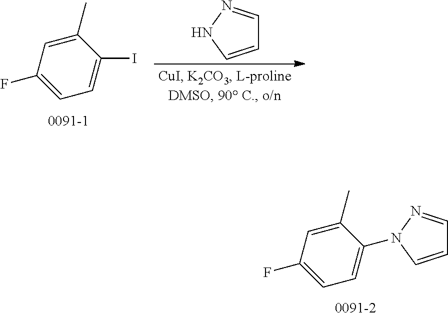

To a stirred solution of 0091-1 (3.0 g, 12.7 mmol) in DMSO (30 ml) was added 1H-pyrazole (1.0 g, 15.3 mmol), CuI (0.30 g), K₂CO₃ (2.6 g, 19.0 mmol) and L-proline (0.90 g). The resulting reaction mixture was stirred at 90° C. for 16 h. Water (30 mL) was added, the aqueous phase was extracted with EA, the combined organic phases were dried over anhydrous sodium sulfate, filtered, and concentrated in vacuo, purified by C.C. to give the desired product 0091-2 (150 mg, yield: 6.7%) as yellow oil.

The synthesis of 4-bromo-1-(4-fluoro-2-methylphenyl)-1H-pyrazole (0091-3).

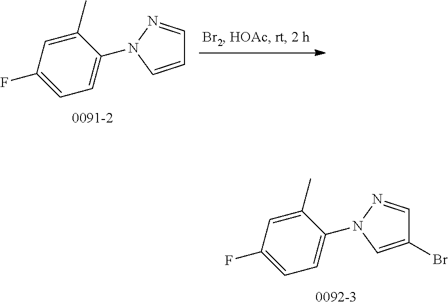

To a stirred solution of 0091-2 (300 mg, 1.7 mmol) in HOAc (10 ml) was added Br₂ (820 mg, 5.1 mmol) slowly. The resulting reaction mixture was stirred at rt for 2 h. Then added water, the aqueous phase was extracted with dichloromethane, the combined organic phases were dried over anhydrous sodium sulfate, filtered and concentrated to give the desired product 0091-3 (300 mg, yield: 70%) as yellow oil.

The synthesis of N-(3-(1-(4-fluoro-2-methylphenyl)-1H-pyrazol-4-yl)phenyl)acetamide (SU20666-0091).

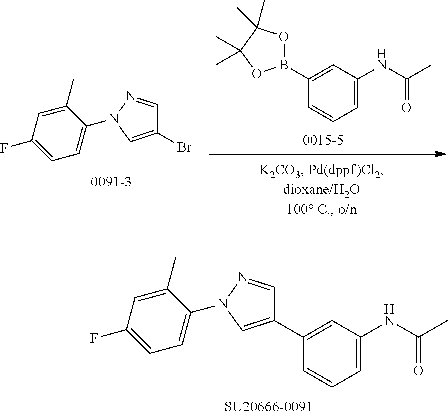 [SMILES: Cc1cc(F)ccc1-n1cc(Br)cn1 ; CC(=O)Nc1cccc(B2OC(C)(C)C(C)(C)O2)c1 ; CC(=O)Nc1cccc(-c2cnn(-c3ccc(F)cc3C)c2)c1]

To a stirred solution of compound 0091-3 (150 mg, 0.59 mmol) in dioxane/water (10 mL/2 mL) was added 015-5 (231 mg, 0.89 mmol), K₂CO₃ (244 mg, 1.77 mmol), Pd(dppf)Cl₂ (20 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0091 (25 mg, yield: 14%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.894 min; MS Calcd.: 309.1; MS Found: 310.4 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water +10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.926 min.

¹H NMR (400 MHz, DMSO-d₆) δ 2.06 (3H, s), 2.25 (3H, s), 7.20 (1H, dt, J=8.8, 2.8 Hz), 7.28-7.35 (3H, m), 7.44-7.49 (2H, m), 7.81 (1H, s), 8.04 (1H, s), 8.40 (1H, s), 9.96 (1H, s).

SU20666-0092

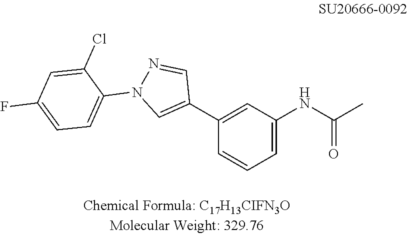 [SMILES: CC(=O)Nc1cccc(-c2cnn(-c3ccc(F)cc3Cl)c2)c1]

Route for SU20666-0092

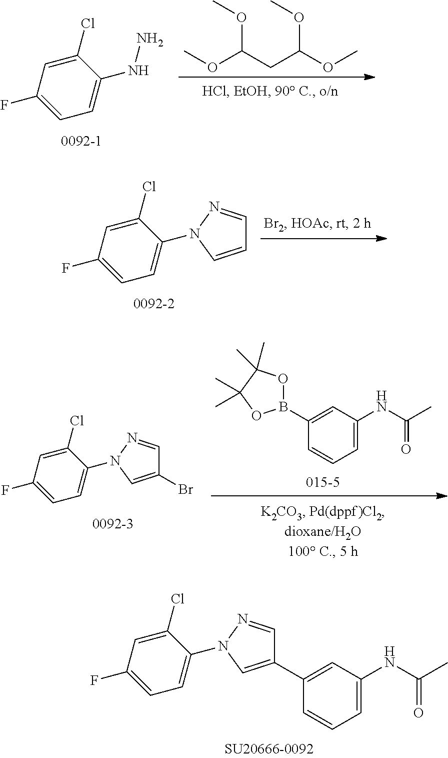

The synthesis of 1-(2-chloro-4-fluorophenyl)-1H-pyrazole (0092-2).

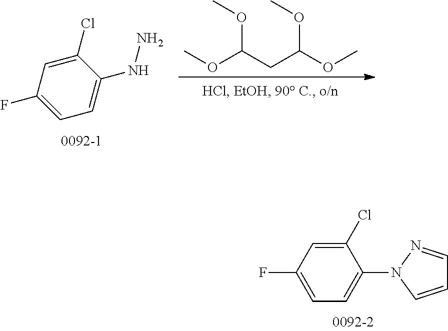

To a stirred solution of 0092-1 (1.0 g, 5.0 mmol) in EtOH (20 ml) was added 1,1,3,3-tetramethoxypropane (1.3 g, 7.6 mmol) and HCl (aq. 10.0 N, 5 mL). The resulting reaction mixture was stirred at 90° C. for 16 h and then concentrated in vacuo, the crude was further purified by C.C. to give the desired product 0092-2 (960 mg, yield: 98%) as yellow oil.

The synthesis of 4-bromo-1-(2-chloro-4-fluorophenyl)-1H-pyrazole (0092-3).

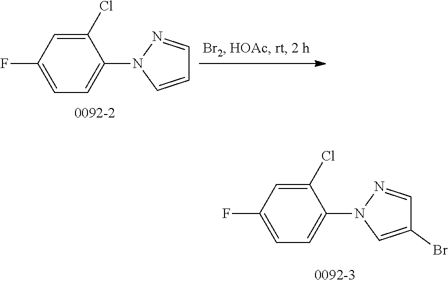 [SMILES: Fc1ccc(-n2cccn2)c(Cl)c1 ; Fc1ccc(-n2cc(Br)cn2)c(Cl)c1]

To a stirred solution of 0092-2 (960 mg, 4.9 mmol) in HOAc (10 ml) was added Br₂ (784 mg, 4.9 mmol) slowly. The resulting reaction mixture was stirred at rt for 2 h. Then added water, the aqueous phase was extracted with dichloromethane, the combined organic phases were dried over anhydrous sodium sulfate, filtered and concentrated, the crude was further purified by C.C. to give the desired product 0092-3 (800 mg, yield: 59%) as a yellow solid.

The synthesis of N-(3-(1-(4-fluoro-2-methylphenyl)-1H-pyrazol-4-yl)phenyl)acetamide (SU20666-0092).

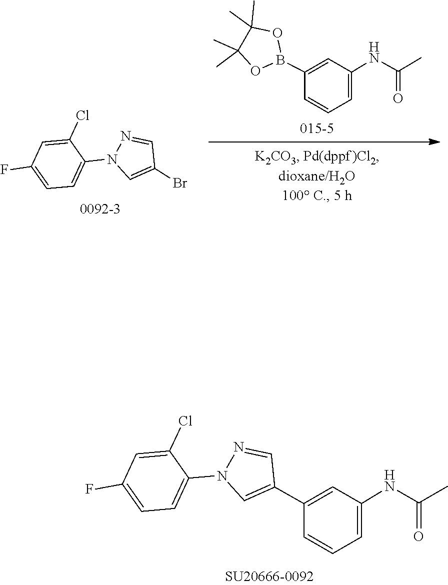 [SMILES: CC(=O)Nc1cccc(B2OC(C)(C)C(C)(C)O2)c1 ; Fc1ccc(-n2cc(Br)cn2)c(Cl)c1 ; CC(=O)Nc1cccc(-c2cnn(-c3ccc(F)cc3Cl)c2)c1]

To a stirred solution of compound 0092-3 (270 mg, 1.0 mmol) in dioxane/water (10 mL/2 mL) was added 015-5 (311 mg, 1.2 mmol), K₂CO₃ (206 mg, 1.5 mmol), Pd(dppf)Cl₂ (70 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0092 (58 mg, yield: 18%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.32%, Rt=1.883 min; MS Calcd.: 329.1; MS Found: 330.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.135 min.

¹H NMR (400 MHz, DMSO-d₆) δ 2.05 (3H, s), 7.29-7.36 (2H, m), 7.41-7.48 (2H, m), 7.71-7.76 (2H, m), 7.81 (1H, s), 8.10 (1H, s), 8.48 (1H, s), 9.98 (1H, s).

SU20666-0093

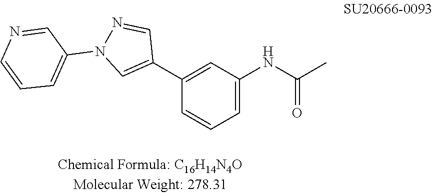 [SMILES: CC(=O)Nc1cccc(-c2cnn(-c3cccnc3)c2)c1]

Route for SU20666-0093

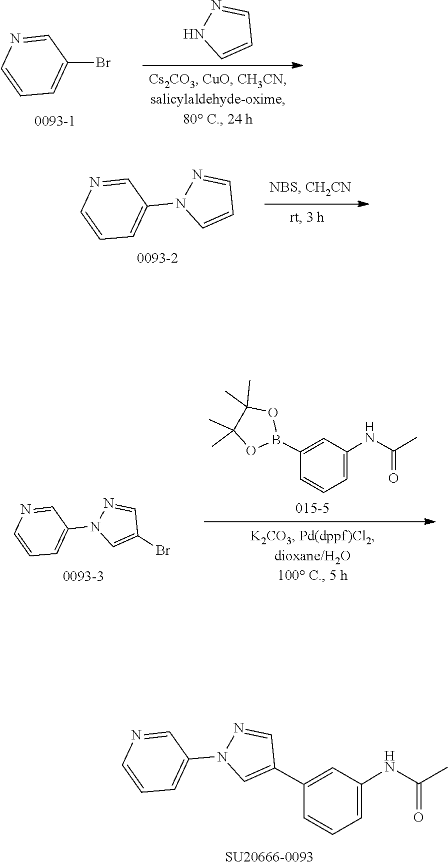 [SMILES: Brc1cccnc1 ; c1cn[nH]c1 ; c1cncc(-n2cccn2)c1 ; CC(=O)Nc1cccc(B2OC(C)(C)C(C)(C)O2)c1 ; Brc1cnn(-c2cccnc2)c1 ; CC(=O)Nc1cccc(-c2cnn(-c3cccnc3)c2)c1]

The synthesis of 3-(1H-pyrazol-1-yl)pyridine (0093-2).

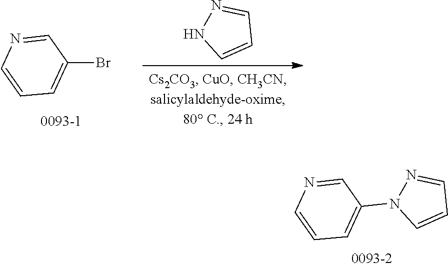 [SMILES: Brc1cccnc1 ; c1cn[nH]c1 ; c1cncc(-n2cccn2)c1]

To a stirred solution of 0093-1 (2.0 g, 12.7 mmol) in acetonitrile (30 ml) was added 1H-pyrazole (1.3 g, 19.1 mmol), Cs₂CO₃ (6.5 g, 20.0 mmol), CuO (0.10 g) and salicylaldehyde-oxime (0.35 g, 2.5 mmol). The resulting reaction mixture was stirred at 80° C. for 24 h. Then added water, the aqueous phase was extracted with EA, the combined organic phases was dried over anhydrous sodium sulfate, filtered, and concentrated in vacuo, thus was further purified by C.C. to give the desired product 0093-2 (0.65 g, yield: 35%) as yellow oil.

The synthesis of 3-(4-bromo-1H-pyrazol-1-yl)pyridine (0093-3).

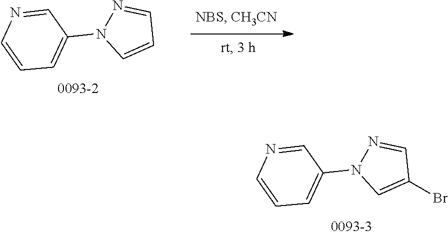 [SMILES: c1cncc(-n2cccn2)c1 ; Brc1cnn(-c2cccnc2)c1]

To a stirred solution of 0093-2 (350 mg, 2.4 mmol) in acetonitrile (8 ml) was added NBS (560 mg, 3.1 mmol). The resulting reaction mixture was stirred at rt for 3 h. Then added water, the aqueous phase was extracted with dichloromethane, the combined organic phases were dried over anhydrous sodium sulfate, filtered and concentrated, the crude was further purified by C.C. to give the desired product 0093-3 (500 mg, yield: 93%) as a yellow solid.

The synthesis of N-(3-(1-(pyridin-3-yl)-1H-pyrazol-4-yl)phenyl)acetamide (SU20666-0093).

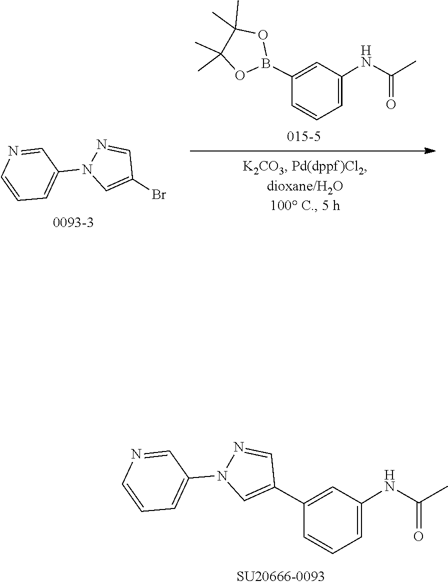 [SMILES: CC(=O)Nc1cccc(B2OC(C)(C)C(C)(C)O2)c1 ; Brc1cnn(-c2cccnc2)c1 ; CC(=O)Nc1cccc(-c2cnn(-c3cccnc3)c2)c1]

To a stirred solution of compound 0093-3 (350 mg, 1.6 mmol) in dioxane/water (10 mL/2 mL) was added 015-5 (615 mg, 2.4 mmol), K₂CO₃ (650 mg, 4.7 mmol), Pd(dppf)Cl₂ (50 mg). The resulting reaction mixture was heated to 100° C. and stirred for 5 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product SU20666-0093 (40 mg, yield: 9.1%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.340 min; MS Calcd.: 278.1; MS Found: 279.3 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 98.38%, Rt=6.634 min.

¹H NMR (400 MHz, DMSO-d₆) δ 2.07 (3H, s), 7.33-7.40 (2H, m), 7.45 (1H, d, J=7.6 Hz), 7.58 (1H, dd, J=8.4, 4.8 Hz), 7.87 (1H, s), 8.17 (1H, s), 8.30 (1H, d, J=8.4 Hz), 8.55 (1H, d, J=4.0 Hz), 9.02 (1H, s), 9.18 (1H, d, J=2.4 Hz), 10.01 (1H, s).

The names SU20666-0094, SP 94, and 94 all refer to the same compound having the formula:

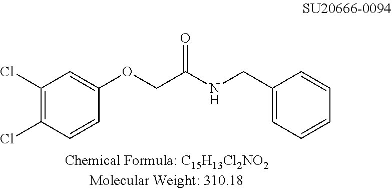 [SMILES: O=C(COc1ccc(Cl)c(Cl)c1)NCc1ccccc1]

Route for SU20666-0094

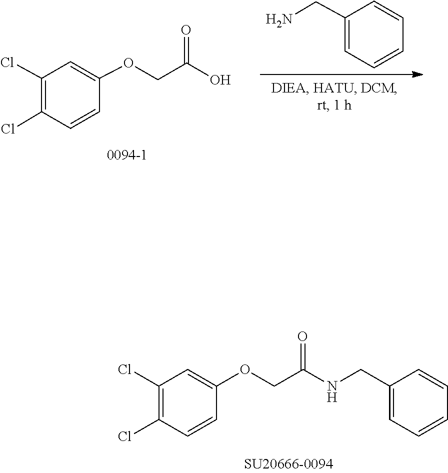 [SMILES: O=C(O)COc1ccc(Cl)c(Cl)c1 ; NCc1ccccc1 ; O=C(COc1ccc(Cl)c(Cl)c1)NCc1ccccc1]

The synthesis of N-benzyl-2-(3,4-dichlorophenoxy)acetamide (SU20666-0094).

To a solution of compound 0094-1 (100 mg, 0.45 mmol) in DCM (5 mL) was added phenylmethanamine (58 mg, 0.54 mmol), DIEA (176 mg, 1.36 mmol) and HATU (259 mg, 0.68 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0094 (20 mg, yield: 14%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=2.139 min; MS Calcd.: 309.0; MS Found: 310.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.328 min.

¹H NMR (400 MHz, DMSO-d₆) δ 4.32 (2H, d, J=6.4 Hz), 4.61 (2H, s), 6.99 (1H, dd, J=8.8, 2.8 Hz), 7.21-7.30 (6H, m), 7.53 (1H, d, J=8.8 Hz), 8.66 (1H, t, J=5.6 Hz).

The names SU20666-0095, SP 95, and 95 all refer to the same compound having the formula:

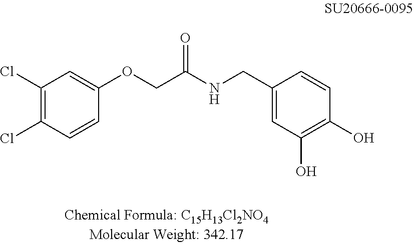 [SMILES: O=C(COc1ccc(Cl)c(Cl)c1)NCc1ccc(O)c(O)c1]

Route for SU20666-0095

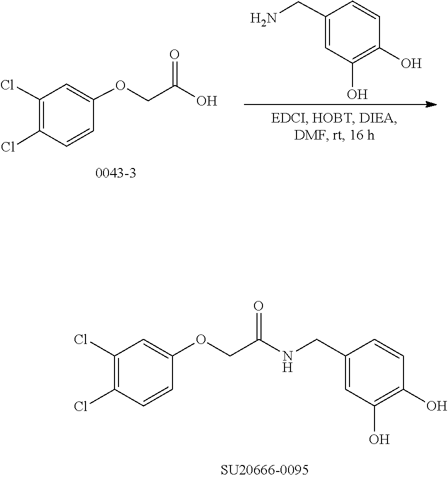 [SMILES: O=C(O)COc1ccc(Cl)c(Cl)c1 ; NCc1ccc(O)c(O)c1 ; O=C(COc1ccc(Cl)c(Cl)c1)NCc1ccc(O)c(O)c1]

The synthesis of 2-(3,4-dichlorophenoxy)-N-(3,4-dihydroxybenzyl)acetamide (SU20666-0095).

To a solution of compound 0043-3 (100 mg, 0.45 mmol) in DMF (5 mL) was added 4-(aminomethyl)benzene-1,2-diol (63 mg, 0.45 mmol), DIEA (174 mg, 1.35 mmol), EDCI (130 mg, 0.67 mmol) and HOBT (91 mg, 0.67 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0095 (15 mg, yield: 9.6%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.650 min; MS Calcd.: 341.0; MS Found: 342.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=8.169 min.

¹H NMR (400 MHz, DMSO-d₆) δ 4.15 (2H, d, J=6.0 Hz), 4.57 (2H, s), 6.49 (1H, dd, J=8.0, 2.0 Hz), 6.63-6.66 (2H, m), 6.99 (1H, dd, J=9.2, 3.2 Hz), 7.26 (1H, d, J=2.8 Hz), 7.54 (1H, d, J=8.8 Hz), 8.52 (1H, t, J=6.0 Hz), 8.74 (1H, s), 8.82 (1H, s).

The names SU20666-0096, SP 96, and 96 all refer to the same compound having the formula:

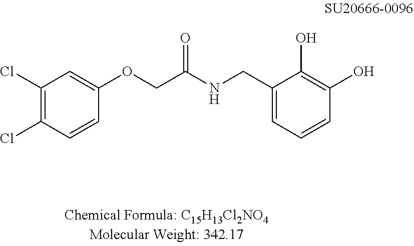 [SMILES: O=C(COc1ccc(Cl)c(Cl)c1)NCc1cccc(O)c1O]

Route for SU20666-0096

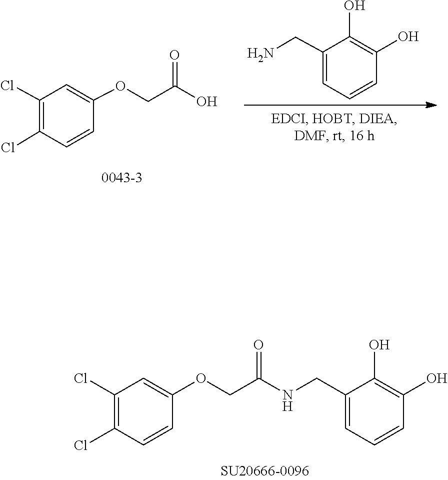 [SMILES: O=C(O)COc1ccc(Cl)c(Cl)c1 ; NCc1cccc(O)c1O ; O=C(COc1ccc(Cl)c(Cl)c1)NCc1cccc(O)c1O]

The synthesis of 2-(3,4-dichlorophenoxy)-N-(2,3-dihydroxybenzyl)acetamide (SU20666-0096).

To a solution of compound 0043-3 (100 mg, 0.45 mmol) in DMF (5 mL) was added 3-(aminomethyl)benzene-1,2-diol (63 mg, 0.45 mmol), DIEA (174 mg, 1.35 mmol), EDCI (173 mg, 0.90 mmol) and HOBT (121 mg, 0.90 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0096 (15 mg, yield: 9.6%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.751 min; MS Calcd.: 341.0; MS Found: 342.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=8.839 min.

¹H NMR (400 MHz, DMSO-d₆) δ 4.27 (2H, d, J=6.0 Hz), 4.62 (2H, s), 6.52-6.58 (2H, m), 6.68 (1H, dd, J=7.6, 2.0 Hz), 7.01 (1H, dd, J=8.8, 2.8 Hz), 7.28 (1H, d, J=3 .2 Hz), 7.55 (1H, d, J=8.8 Hz), 8.52 (1H, t, J=5.6 Hz), 8.60 (1H, s), 9.19 (1H, s).

The names SU20666-0097, SP 97, and 97 all refer to the same compound having the formula:

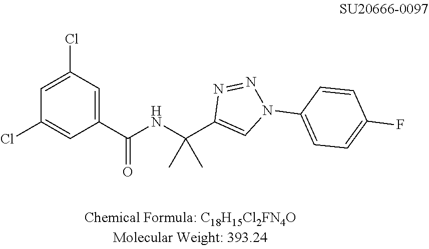 [SMILES: CC(C)(NC(=O)c1cc(Cl)cc(Cl)c1)c1cn(-c2ccc(F)cc2)nn1]

Route for SU20666-0097

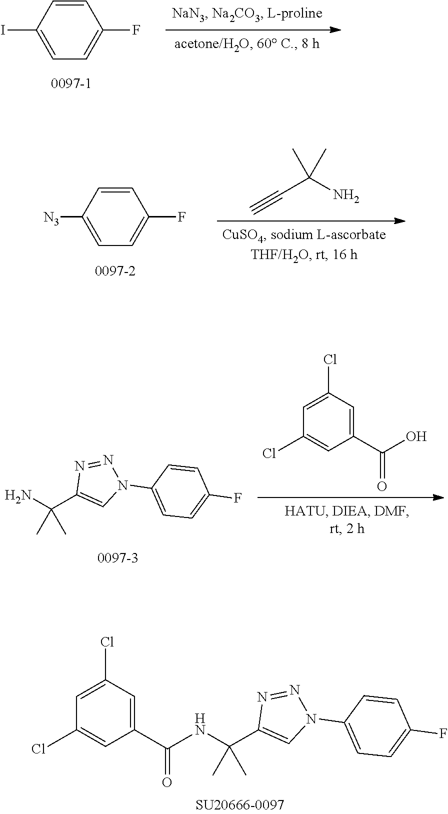

The synthesis of 1-azido-4-fluorobenzene (0097-2).

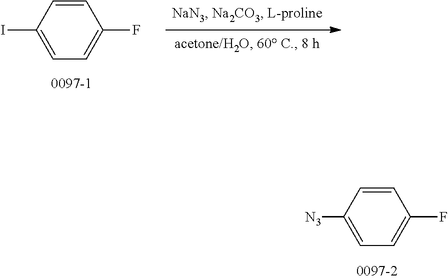 [SMILES: Fc1ccc(I)cc1 ; [N-]=[N+]=Nc1ccc(F)cc1]

To a stirred solution of 0097-1 (500 mg, 2.3 mmol) in acetone/H₂O (20 ml/3 mL) was added sodium azide (176 mg, 2.7 mmol), Na₂CO3 (49 mg, 0.45 mmol) and L-proline (52 mg, 0.45 mmol). The resulting reaction mixture was stirred at 60° C. for 8 h. Then added water, the aqueous phase was extracted with dichloromethane, the combined organic phases were dried over anhydrous sodium sulfate, filtered, and concentrated in vacuo to give the desired product 0097-2 (290 mg, yield: 94%) as colorless oil.

The synthesis of 2-(1-(4-fluorophenyl)-1H-1,2,3-triazol-4-yl)propan-2-amine (0097-3).

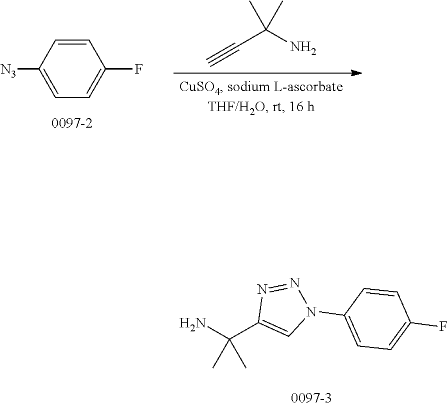

To a solution of compound 0097-2 (290 mg, 2.1 mmol) in THF/H₂O (20 mL/4 mL) was added copper sulfate pentahydrate (523 mg, 2.1 mmol), sodium L-ascorbate (220 mg, 1.1 mmol) and 2-methylbut-3-yn-2-amine (174 mg, 2.1 mmol) at 0° C. The resulting reaction mixture was stirred for 16 h at rt, then added water, the aqueous phase was extracted with DCM, then the aqueous phases was concentrated to give the desired product 0097-2 (160 mg, yield: 34%) as yellow oil, which used to the next step without further purification.

The synthesis of 3,5-dichloro-N-(2-(1-(4-fluorophenyl)-1H-1,2,3-triazol-4-yl)propan-2-yl)benzamide (SU20666-0097).

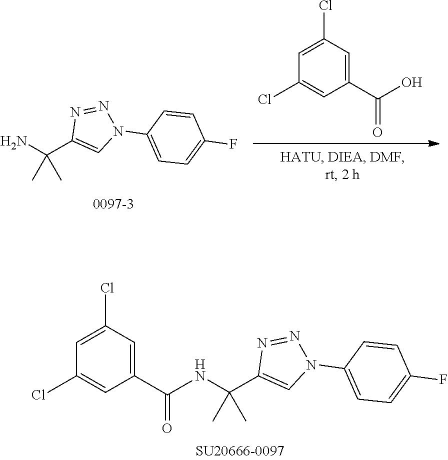

To a solution of compound 0097-3 (160 mg, 0.73 mmol) in DMF (5 mL) was added 3,5-dichlorobenzoic acid (63 mg, 0.73 mmol), DIEA (283 mg, 2.20 mmol) and HATU (466 mg, 1.10 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0097 (130 mg, yield: 42%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 94.83%, Rt=2.364 min; MS Calcd.: 392.1; MS Found: 393.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.30%, Rt=10.888 min.

¹H NMR (400 MHz, DMSO-d₆) δ 1.76 (6H, s), 7.45 (2H, t, J=8.8 Hz), 7.80 (1H, d, J=2.0 Hz), 7.87 (2H, d, J=2.0 Hz), 7.96 (2H, dd, J=9.2, 4.8 Hz), 8.68 (1H, s), 8.72 (1H, s).

The names SU20666-0098, SP 98, and 98 all refer to the same compound having the formula:

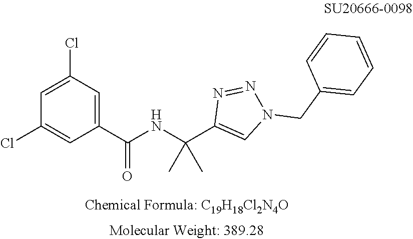 [SMILES: CC(C)(NC(=O)c1cc(Cl)cc(Cl)c1)c1cn(Cc2ccccc2)nn1]

Route for SU20666-0098

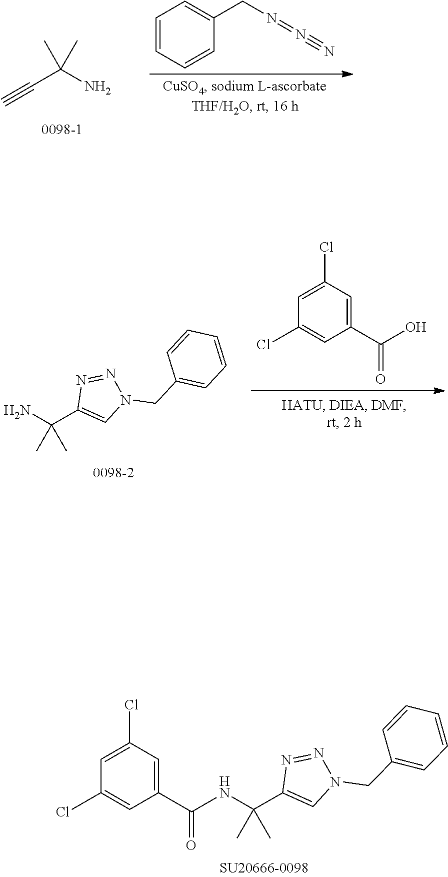

The synthesis of 2-(1-benzyl-1H-1,2,3-triazol-4-yl)propan-2-amine (0098-2).

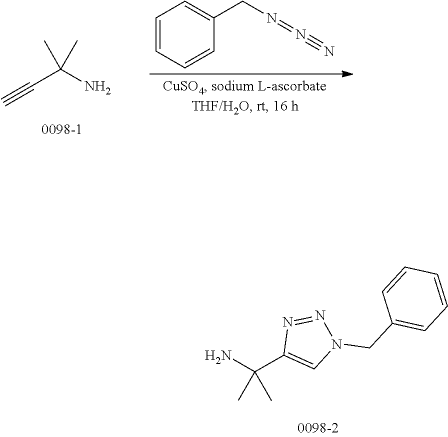

To a solution of compound 0098-2 (500 mg, 6.0 mmol) in THF/H₂O (20 mL/4 mL) was added copper sulfate pentahydrate (750 mg, 3.0 mmol), sodium L-ascorbate (600 mg, 3.0 mmol) and (azidomethyl)benzene (800 mg, 6.0 mmol) at 0° C. The resulting reaction mixture was stirred for 16 h at rt, then added water, the aqueous phase was extracted with DCM, then the aqueous phases was concentrated to give the desired product 0098-2 (600 mg, crude) as yellow oil, which used to the next step without further purification.

The synthesis of N-(2-(1-benzyl-1H-1,2,3-triazol-4-yl)propan-2-yl)-3,5-dichlorobenzamide (SU20666-0098).

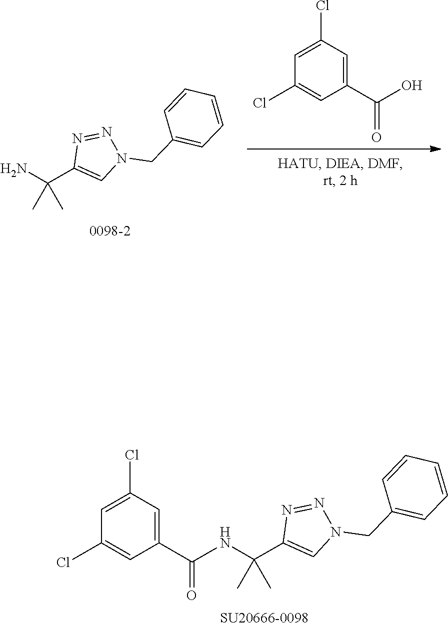

To a solution of compound 0098-2 (500 mg, 1.3 mmol) in DMF (5 mL) was added 3,5-dichlorobenzoic acid (240 mg, 1.3 mmol), DIEA (484 mg, 3.8 mmol) and HATU (714 mg, 1.9 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0098 (110 mg, yield: 19%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.53%, Rt=2.140 min; MS Calcd.: 388.1; MS Found: 389.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100%

[CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.357 min.

¹H NMR (400 MHz, DMSO-d₆) δ 1.69 (6H, s), 5.54 (2H, s), 7.29-7.37 (5H, m), 7.78 (1H, t, J=2.0 Hz), 7.83 (2H, d, J=2.0 Hz), 8.01 (1H, s), 8.60 (1H, s).

The names SU20666-0099, SP 99, and 99 all refer to the same compound having the formula:

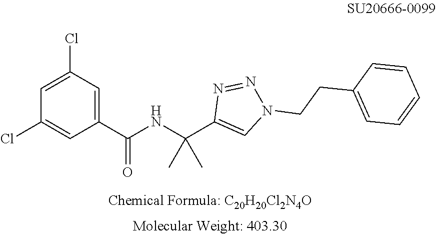 [SMILES: CC(C)(NC(=O)c1cc(Cl)cc(Cl)c1)c1cn(CCc2ccccc2)nn1]

Route for SU20666-0099

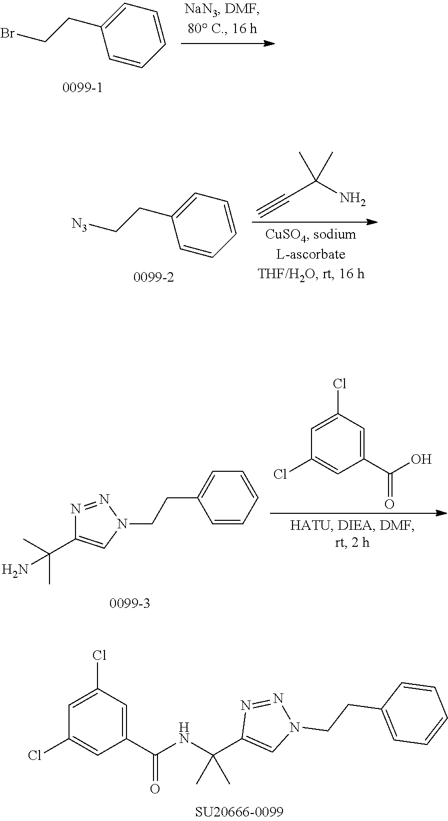

The synthesis of (2-azidoethyl)benzene (0099-2).

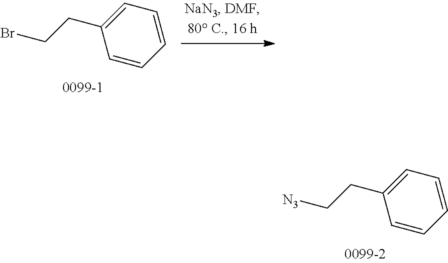 [SMILES: BrCCc1ccccc1 ; [N-]=[N+]=NCCc1ccccc1]

To a stirred solution of 0099-1 (0.60 g, 3.3 mmol) in DMF (10 ml) was added sodium azide (0.43 g, 6.6 mmol). The resulting reaction mixture was stirred at 80° C. for 16 h. Then added water, the aqueous phase was extracted with EA, the combined organic phases were dried over anhydrous sodium sulfate, filtered, and concentrated in vacuo to give the desired product 0099-2 (0.40 g, yield: 83%) as yellow oil.

The synthesis of 2-(1-phenethyl-1H-1,2,3-triazol-4-yl)propan-2-amine (0099-3).

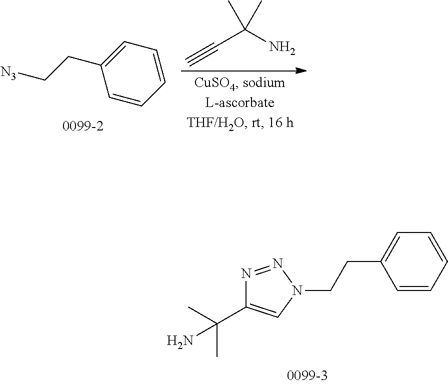

To a solution of compound 0099-2 (0.40 g, 2.7 mmol) in THF/H₂O (20 mL/4 mL) was added copper sulfate pentahydrate (0.67 g, 2.7 mmol), sodium L-ascorbate (0.27 g, 1.3 mmol) and 2-methylbut-3-yn-2-amine (0.23 g, 2.7 mmol) at 0° C. The resulting reaction mixture was stirred for 16 h at rt, then added water, the aqueous phase was extracted with DCM, then the aqueous phases was concentrated to give the desired product 0099-3 (0.12 g, yield: 19%) as yellow oil, which used to the next step without further purification.

The synthesis of 3,5-dichloro-N-(2-(1-phenethyl-1H-1,2,3-triazol-4-yl)propan-2-yl)benzamide (SU20666-0099).

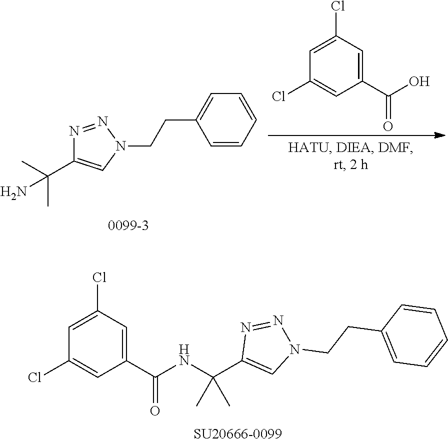

To a solution of compound 0099-3 (120 mg, 0.52 mmol) in DMF (5 mL) was added 3,5-dichlorobenzoic acid (99 mg, 0.52 mmol), DIEA (200 mg, 1.56 mmol) and HATU (300 mg, 0.78 mmol). The resulting reaction mixture was stirred for 2 h at rt, then added water, the aqueous phase was extracted with EA, the combined organic phases were dried over anhydrous sodium sulfate, filtered, and concentrated, the crude was purified by prep-HPLC to give the desired product SU20666-0099 (56 mg, yield: 27%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.29%, Rt=2.164 min; MS Calcd.: 402.1; MS Found: 403.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 96.07%, Rt=10.782 min.

¹H NMR (400 MHz, DMSO-d₆) δ 1.67 (6H, s), 3.12 (2H, t, J=7.6 Hz), 4.53 (2H, t, J=7.6 Hz), 7.16-7.25 (5H, m), 7.79 (1H, t, J=2.0 Hz), 7.83-7.85 (3H, m), 8.01 (1H, s), 8.58(1H, s).

The names SU20666-0100, SP 100, and 100 all refer to the same compound having the formula:

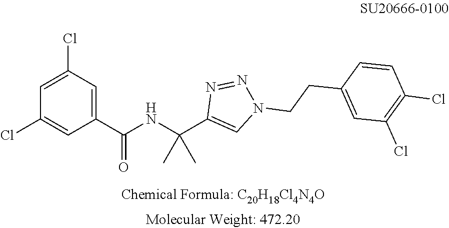 [SMILES: CC(C)(NC(=O)c1cc(Cl)cc(Cl)c1)c1cn(CCc2ccc(Cl)c(Cl)c2)nn1]

Route for SU20666-0100

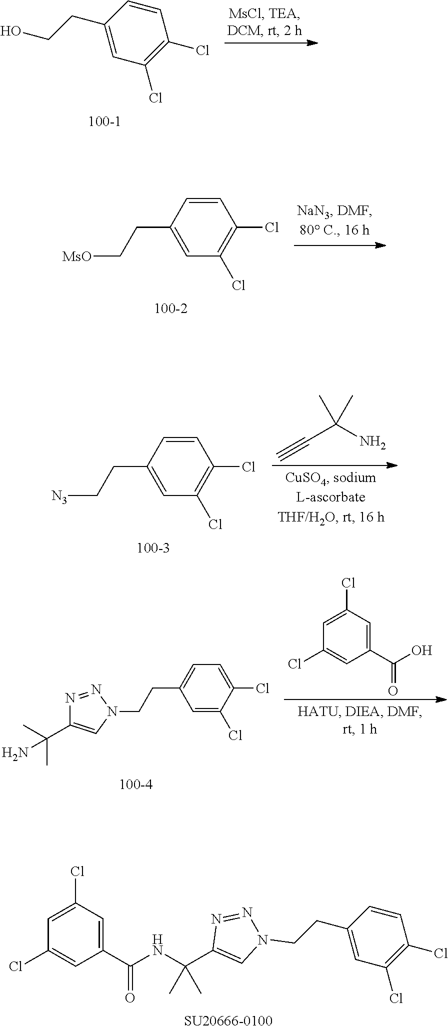

The synthesis of 3,4-dichlorophenethyl methanesulfonate (100-2).

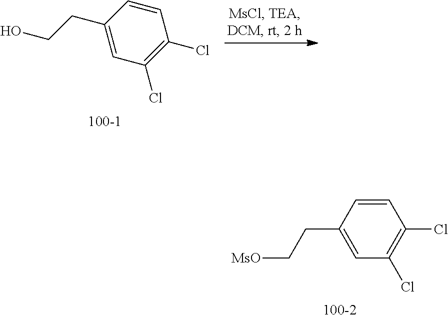 [SMILES: OCCc1ccc(Cl)c(Cl)c1 ; CS(=O)(=O)OCCc1ccc(Cl)c(Cl)c1]

To a stirred solution of 100-1 (800 mg, 4.2 mmol) in DCM (10 ml) was added TEA (850 mg, 8.4 mmol) and MsCl (720 mg, 6.3 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo to give the desired product 100-2 (1.0 g, yield: 89%) as yellow oil.

The synthesis of 4-(2-azidoethyl)-1,2-dichlorobenzene (100-3).

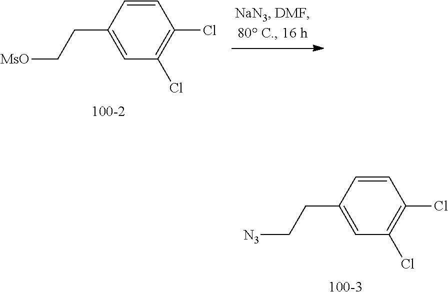 [SMILES: CS(=O)(=O)OCCc1ccc(Cl)c(Cl)c1 ; [N-]=[N+]=NCCc1ccc(Cl)c(Cl)c1]

To a stirred solution of 100-2 (1.0 g, 3.7 mmol) in DMF (10 ml) was added sodium azide (0.49 g, 7.4 mmol). The resulting reaction mixture was stirred at 80° C. for 16 h. Then added water, the aqueous phase was extracted with EA, the combined organic phases were dried over anhydrous sodium sulfate, filtered, and concentrated in vacuo to give the desired product 100-3 (0.70 g, yield: 87%) as a yellow solid.

The synthesis of 2-(1-(3,4-dichlorophenethyl)-1H-1,2,3-triazol-4-yl)propan-2-amine (100-4).

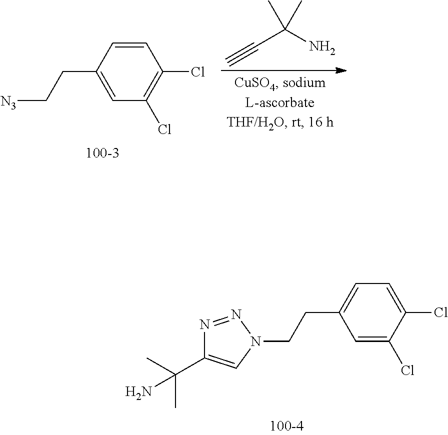

To a solution of compound 100-3 (0.40 g, 1.9 mmol) in THF/H₂O (20 mL/4 mL) was added copper sulfate pentahydrate (0.24 g, 0.95 mmol), sodium L-ascorbate (0.19 g, 0.95 mmol) and 2-methylbut-3-yn-2-amine (0.15 g, 1.9 mmol) at 0° C. The resulting reaction mixture was stirred for 16 h at rt, then added water, the aqueous phase was extracted with DCM, then the aqueous phases was concentrated to give the desired product 100-4 (0.36 g, yield: 65%) as yellow oil, which used to the next step without further purification.

The synthesis of 3,5-dichloro-N-(2-(1-(3,4-dichlorophenethyl)-1H-1,2,3-triazol-4-yl)propan-2-yl)benzamide (SU20666-0100).

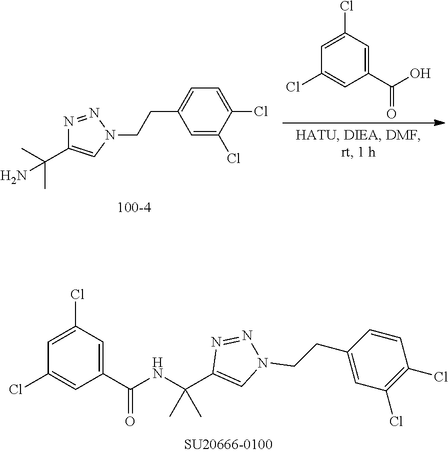 [SMILES: CC(C)(N)c1cn(CCc2ccc(Cl)c(Cl)c2)nn1 ; O=C(O)c1cc(Cl)cc(Cl)c1 ; CC(C)(NC(=O)c1cc(Cl)cc(Cl)c1)c1cn(CCc2ccc(Cl)c(Cl)c2)nn1]

To a solution of compound 100-4 (200 mg, 0.67 mmol) in DMF (10 mL) was added 3,5-dichlorobenzoic acid (130 mg, 0.67 mmol), DIEA (260 mg, 2.01 mmol) and HATU (380 mg, 1.0 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product SU20666-0100 (100 mg, yield: 32%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.26%, Rt=2.325 min; MS Calcd.: 470.0; MS Found: 471.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100%

[CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.486 min.

¹H NMR (400 MHz, DMSO-d₆) δ 1.67 (6H, s), 3.13 (2H, t, J=7.2 Hz), 4.54 (2H, t, J=7.2 Hz), 7.11 (1H, dd, J=8.0, 2.0 Hz), 7.40 (1H, d, J=1.6 Hz), 7.46 (1H, d, J=8.4 Hz), 7.80 (1H, d, J=1.6 Hz), 7.84-7.87 (3H, m), 8.58 (1H, s).

The names SU20666-0102, SP 102, and 102 all refer to the same compound having the formula:

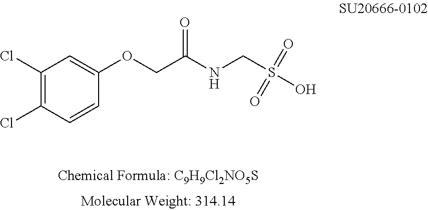 [SMILES: O=C(COc1ccc(Cl)c(Cl)c1)NCS(=O)(=O)O]

Route for SU20666-0102

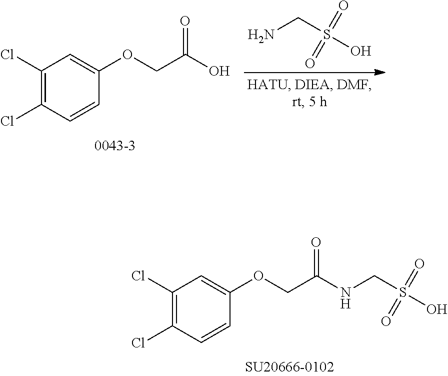 [SMILES: O=C(O)COc1ccc(Cl)c(Cl)c1 ; NCS(=O)(=O)O ; O=C(COc1ccc(Cl)c(Cl)c1)NCS(=O)(=O)O]

The synthesis of (2-(3,4-dichlorophenoxy)acetamido)methanesulfonic acid (SU20666-0102).

To a solution of compound 0043-4 (200 mg, 0.91 mmol) in DMF (6 mL) was added aminomethanesulfonic acid (121 mg, 1.10 mmol), DIEA (350 mg, 2.70 mmol) and HATU (530 mg, 1.4 mmol). The resulting reaction mixture was stirred for 5 h at rt, then added water, the aqueous phase was extracted with EA, the combined organic phases were dried over anhydrous sodium sulfate, filtered, and concentrated, the crude was purified by prep-HPLC to give the desired product SU20666-0102 (110 mg, yield: 38%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.417 min; MS Calcd.: 313.0; MS Found: 312.0 [M−H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 97.08%, Rt=6.392 min.

¹H NMR (400 MHz, CD₃OD) 6 4.39 (2H, s), 4.59 (2H, s), 6.97 (1H, dd, J=8.8, 2.8 Hz), 7.22 (1H, d, J=3 .2 Hz), 7.42 (1H, d, J=9.2 Hz).

SU20666-0103

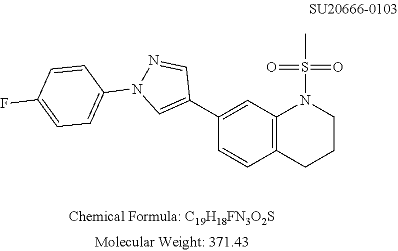 [SMILES: CS(=O)(=O)N1CCCc2ccc(-c3cnn(-c4ccc(F)cc4)c3)cc21]

Route for SU20666-0103

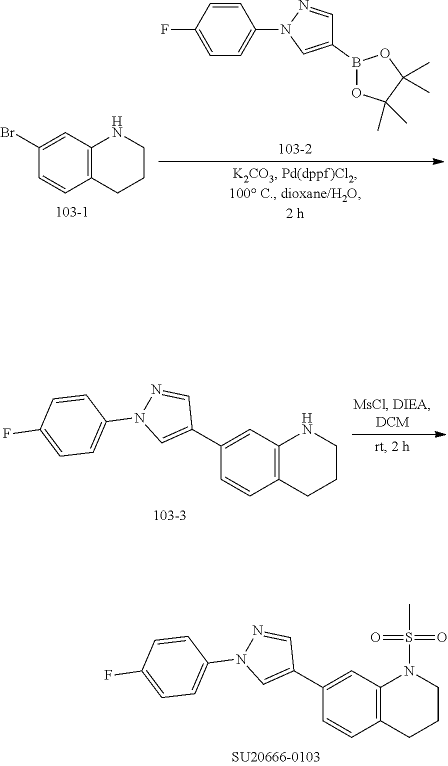 [SMILES: CC1(C)OB(c2cnn(-c3ccc(F)cc3)c2)OC1(C)C ; Brc1ccc2c(c1)NCCC2 ; Fc1ccc(-n2cc(-c3ccc4c(c3)NCCC4)cn2)cc1 ; CS(=O)(=O)N1CCCc2ccc(-c3cnn(-c4ccc(F)cc4)c3)cc21]

The synthesis of 7-(1-(4-fluorophenyl)-1H-pyrazol-4-yl)-1,2,3,4-tetrahydroquinoline (103-3).

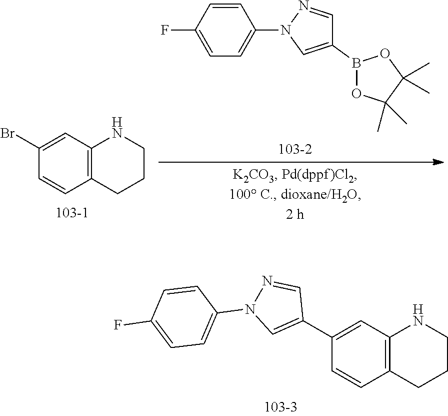 [SMILES: CC1(C)OB(c2cnn(-c3ccc(F)cc3)c2)OC1(C)C ; Brc1ccc2c(c1)NCCC2 ; Fc1ccc(-n2cc(-c3ccc4c(c3)NCCC4)cn2)cc1]

To a stirred solution of compound 103-1 (260 mg, 1.23 mmol) in dioxane/water (10 mL/2 mL) was added 103-2 (530 mg, 1.84 mmol), K₂CO₃ (508 mg, 3.68 mmol), Pd(dppf)Cl₂ (50 mg). The resulting reaction mixture was heated to 100° C. and stirred for 2 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product 103-3 (150 mg, yield: 42%) as yellow oil.

The synthesis of 7-(1-(4-fluorophenyl)-1H-pyrazol-4-yl)-1-(methylsulfonyl)-1,2,3,4-tetrahydroquinoline (SU20666-0103).

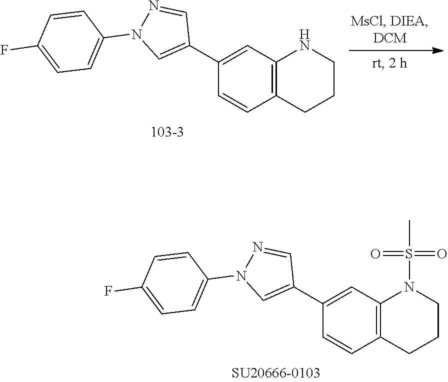 [SMILES: Fc1ccc(-n2cc(-c3ccc4c(c3)NCCC4)cn2)cc1 ; CS(=O)(=O)N1CCCc2ccc(-c3cnn(-c4ccc(F)cc4)c3)cc21]

To a stirred solution of 103-3 (150 mg, 0.50 mmol) in DCM (3 ml) was added DIEA (197 mg, 1.53 mmol) and MsCl (88 mg, 0.77 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo, the crude was further purified by prep-HPLC to give the desired product SU20666-0103 (12 mg, yield: 6.4%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.04%, Rt=2.124 min; MS Calcd.: 371.1; MS Found: 372.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.483 min.

¹H NMR (400 MHz, DMSO-d₆) δ 1.94 (2H, t, J=6.0 Hz), 2.80 (2H, t, J=6.8 Hz), 3.10 (3H, s), 3,71 (2H, t, J=6.0 Hz), 7.22 (1H, d, J=8.0 Hz), 7.36-7.43 (3H, m), 7.77 (1H, s), 7.92-7.95 (2H, m), 8.11 (1H, s), 8.90 (1H, s).

SU20666-0104

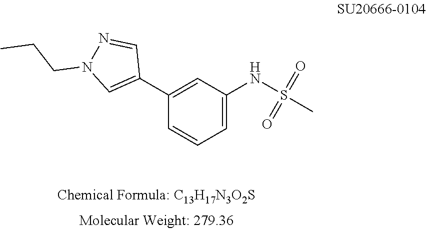 [SMILES: CCCn1cc(-c2cccc(NS(C)(=O)=O)c2)cn1]

Route for SU20666-0104

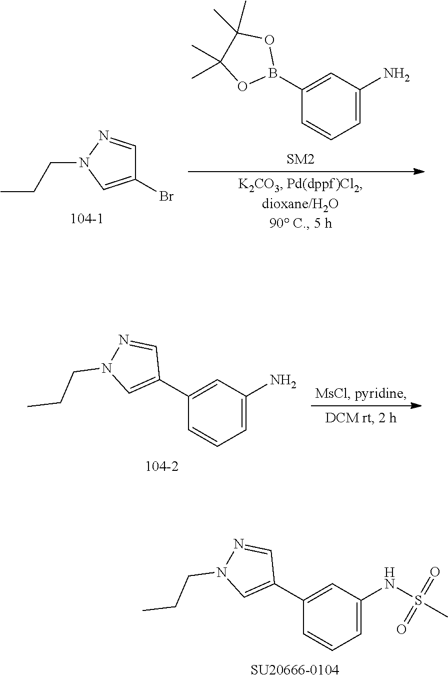

The synthesis of 3-(1-propyl-1H-pyrazol-4-yl)aniline (104-2).

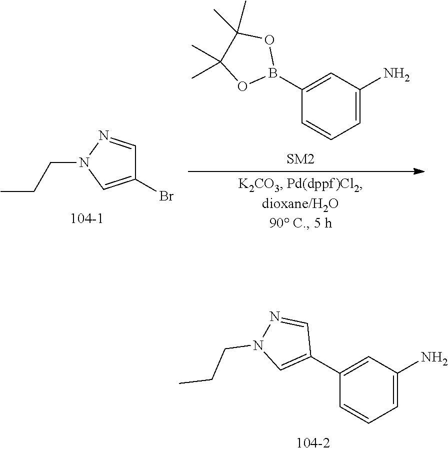

To a stirred solution of compound 104-1 (300 mg, 1.6 mmol) in dioxane/water (20 mL/2 mL) was added SM2 (420 mg, 1.9 mmol), K₂CO₃ (442 mg, 3.2 mmol), Pd(dppf)Cl₂ (50 mg). The resulting reaction mixture was heated to 100° C. and stirred for 2 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 104-2 (240 mg, yield: 75%) as a yellow solid.

The synthesis of N-(3-(1-propyl-1H-pyrazol-4-yl)phenyl)methanesulfonamide (SU20666-0104).

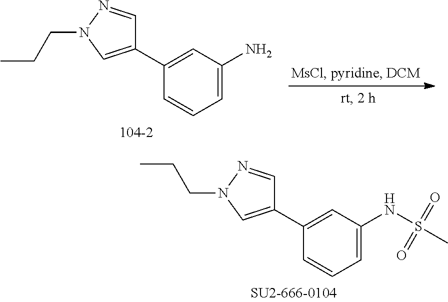 [SMILES: CCCn1cc(-c2cccc(N)c2)cn1 ; CCCn1cc(-c2cccc(NS(C)(=O)=O)c2)cn1]

To a stirred solution of 104-2 (200 mg, 1.0 mmol) in DCM (3 ml) was added pyridine (240 mg, 3.0 mmol) and MsCl (115 mg, 1.0 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo, the crude was further purified by prep-HPLC to give the desired product SU20666-0104 (70 mg, yield: 25%) as a yellow solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.70%, Rt=1.590 min; MS Calcd.: 279.1; MS Found: 280.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.791 min.

¹H NMR (400 MHz, CD₃OD) 6 0.94 (3H, t, J=7.2 Hz), 1.89-1.94 (2H, m), 2.99 (3H, s), 4.15 (2H, t, J=7.6 Hz), 7.12-7.14 (1H, m), 7.32-7.37 (2H, m), 7.42 (1H, s), 7.83 (1H, s), 8.01 (1H, s).

SU20666-0105

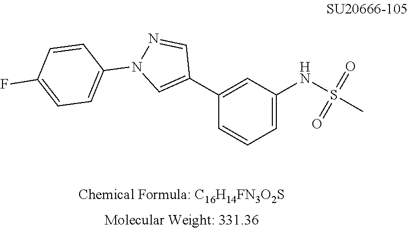 [SMILES: CS(=O)(=O)Nc1cccc(-c2cnn(-c3ccc(F)cc3)c2)c1]

Route for SU20666-0105

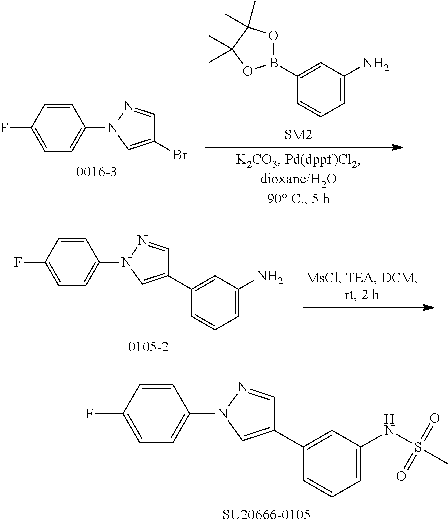 [SMILES: Fc1ccc(-n2cc(Br)cn2)cc1 ; CC1(C)OB(c2cccc(N)c2)OC1(C)C ; Nc1cccc(-c2cnn(-c3ccc(F)cc3)c2)c1 ; CS(=O)(=O)Nc1cccc(-c2cnn(-c3ccc(F)cc3)c2)c1]

The synthesis of 3-(1-(4-fluorophenyl)-1H-pyrazol-4-yl)aniline (105-2).

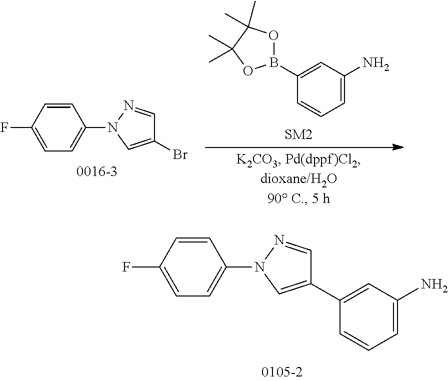 [SMILES: Fc1ccc(-n2cc(Br)cn2)cc1 ; CC1(C)OB(c2cccc(N)c2)OC1(C)C ; Nc1cccc(-c2cnn(-c3ccc(F)cc3)c2)c1]

To a stirred solution of compound 0016-3 (500 mg, 2.1 mmol) in dioxane/water (20 mL/2 mL) was added SM2 (547 mg, 2.5 mmol), K₂CO₃ (427 mg, 3.1 mmol), Pd(dppf)Cl₂ (150 mg). The resulting reaction mixture was heated to 100° C. and stirred for 2 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 105-2 (450 mg, yield: 86%) as a yellow solid.

The synthesis of N-(3-(1-(4-fluorophenyl)-1H-pyrazol-4-yl)phenyl)methanesulfonamide (SU20666-0105).

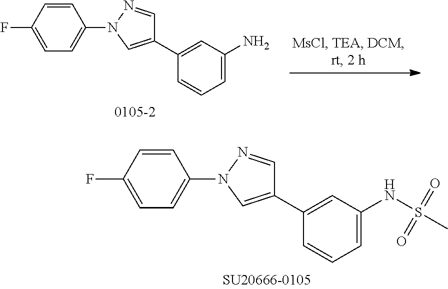 [SMILES: Nc1cccc(-c2cnn(-c3ccc(F)cc3)c2)c1 ; CS(=O)(=O)Nc1cccc(-c2cnn(-c3ccc(F)cc3)c2)c1]

To a stirred solution of 105-2 (100 mg, 0.39 mmol) in DCM (5 ml) was added TEA (59 mg, 0.59 mmol) and MsCl (49 mg, 0.43 mmol) at 0° C. The resulting reaction mixture was stirred for 2 h at rt. Then added water, the aqueous phase was extracted with dichloromethane, the combined organic phases were dried over anhydrous sodium sulfate, filtered, and concentrated in vacuo, the crude was further purified by prep-HPLC to give the desired product SU20666-0105 (23 mg, yield: 18%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.877 min; MS Calcd.: 331.1; MS Found: 332.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.188 min.

¹H NMR (400 MHz, DMSO-d₆) δ 3.04 (3H, s), 7.11 (1H, d, J=7.6 Hz), 7.36-7.41 (3H, m), 7.45-7.47 (2H, m), 7.92-7.96 (2H, m), 8.13 (1H, s), 8.94 (1H, s), 9.77 (1H, s).

SU20666-0106

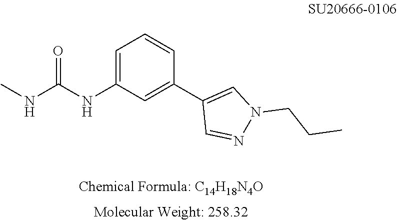 [SMILES: CCCn1cc(-c2cccc(NC(=O)NC)c2)cn1]

Route for SU20666-0106

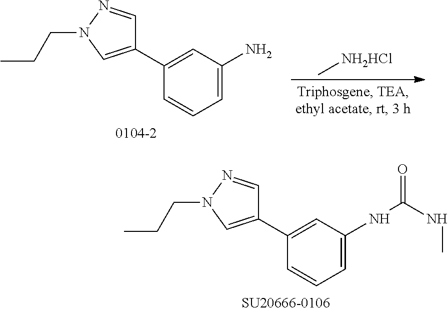

The synthesis of 1-methyl-3-(3-(1-propyl-1H-pyrazol-4-yl)phenyl)urea (SU20666-0106).

To a stirred solution of compound 0104-2 (200 mg, 1.0 mmol) in ethyl acetate (10 mL) was added triphosgene (445 mg, 1.5 mmol), TEA (202 mg, 2.0 mmol) and methanamine hydrochloride (100 mg, 1.5 mmol). The resulting reaction mixture was stirred for 3 h and concentrated in vacuo to remove the solvent, then added water, the aqueous phase was extracted with EA, the combined organic phases were dried over anhydrous sodium sulfate, filtered, and concentrated, the crude was purified by prep-HPLC to give the desired product SU20666-0106 (100 mg, yield: 39%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.15%, Rt=1.535 min; MS Calcd.: 258.1; MS Found: 259.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.404 min.

¹H NMR (400 MHz, DMSO-d₆) 0.84 (3H, t, J=7.2 Hz), 1.78-1.83 (2H, m), 2.64 (3H, d, J=4.4 Hz), 4.07 (2H, t, J=6.8 Hz), 6.03 (1H, d, J=4.4 Hz), 7.07-7.09 (1H, m), 7.16-7.19 (2H, m), 7.58 (1H, s), 7.74 (1H, s), 8.06 (1H, s), 8.48 (1H, s).

SU20666-0107

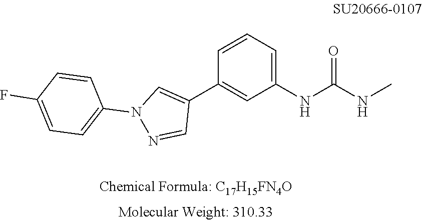 [SMILES: CNC(=O)Nc1cccc(-c2cnn(-c3ccc(F)cc3)c2)c1]

Route for SU20666-0107

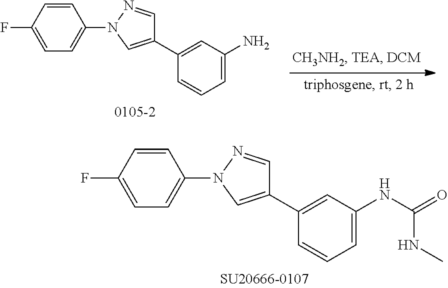 [SMILES: Nc1cccc(-c2cnn(-c3ccc(F)cc3)c2)c1 ; CNC(=O)Nc1cccc(-c2cnn(-c3ccc(F)cc3)c2)c1]

The synthesis of 1-(3-(1-(4-fluorophenyl)-1H-pyrazol-4-yl)phenyl)-3-methylurea (SU20666-0107).

To a stirred solution of compound 0105-2 (100 mg, 0.39 mmol) in DCM (10 mL) was added triphosgene (116 mg, 0.39 mmol), TEA (59 mg, 0.59 mmol) and methanamine hydrochloride (53 mg, 0.78 mmol). The resulting reaction mixture was stirred for 2 h and concentrated in vacuo to remove the solvent, then added water, the aqueous phase was extracted with EA, the combined organic phases were dried over anhydrous sodium sulfate, filtered, and concentrated, the crude was purified by prep-HPLC to give the desired product SU20666-0107 (56 mg, yield: 46%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.781 min; MS Calcd.: 310.1; MS Found: 311.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.675 min.

¹H NMR (400 MHz, DMSO-d₆) 2.66 (3H, d, J=4.4 Hz), 6.07 (1H, d, J=4.4 Hz), 7.23-7.30 (3H, m), 7.38 (2H, t, J=8.8 Hz), 7.69 (1H, s), 7.92-7.96 (2H, m), 8.09 (1H, s), 8.52 (1H, s), 8.88 (1H, s).

The names SU20666-0108, SP 108, and 108 all refer to the same compound having the formula as shown below. The names SU20666-0120, SP 120, and 120 all refer to the same compound having the formula as shown below.

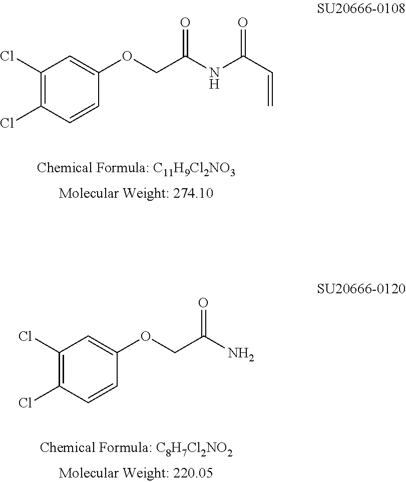 [SMILES: C=CC(=O)NC(=O)COc1ccc(Cl)c(Cl)c1 ; NC(=O)COc1ccc(Cl)c(Cl)c1]

Route for SU20666-0108 and SU20666-0120

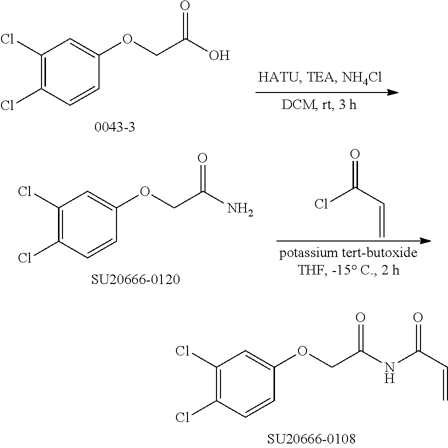

The synthesis of 2-(3,4-dichlorophenoxy)acetamide (SU20666-0120).

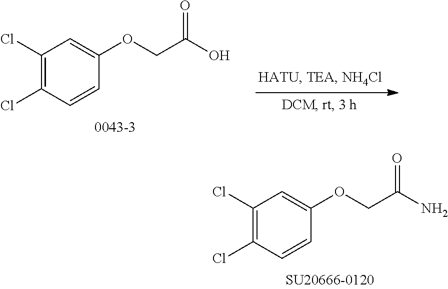 [SMILES: O=C(O)COc1ccc(Cl)c(Cl)c1 ; NC(=O)COc1ccc(Cl)c(Cl)c1]

To a solution of compound 0043-3 (1.0 g, 4.5 mmol) in DCM (30 mL) was added NH₄Cl (294 mg, 5.5 mmol), DIEA (1.8 g, 13.6 mmol) and HATU (2.6 g, 6.8 mmol). The resulting reaction mixture was stirred for 3 h at rt, then added water, the aqueous phase was extracted with DCM, the combined organic phases were dried over anhydrous sodium sulfate, filtered, and concentrated, the crude was purified by prep-HPLC to give the desired product SU20666-0120 (863 mg, yield: 87%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.582 min; MS Calcd.: 219.1; MS Found: 220.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=7.783 min.

¹H NMR (400 MHz, DMSO-d₆) 4.48 (2H, s), 6.98 (1H, dd, J=8.8, 2.8 Hz), 7.24 (1H, d, J=2.8 Hz), 7.42 (1H, s), 7.53-7.56 (2H, m).

The synthesis of N-(2-(3,4-dichlorophenoxy)acetyl)acrylamide (SU20666-0108).

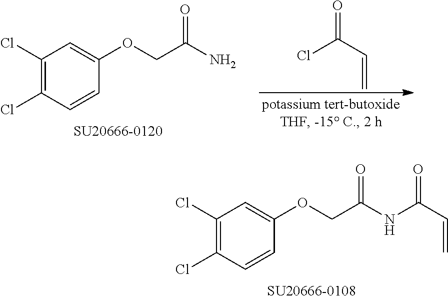

To a solution of compound SU20666-0120 (200 mg, 0.91 mmol) in THF (6 mL) was added potassium tert-butoxide (205 mg, 1.82 mmol) at −15° C. and stirred at this temperature for 30 min, then added acryloyl chloride (123 mg, 1.37 mmol). The resulting reaction mixture was stirred for 2 h at −15° C., then added water, the aqueous phase was extracted with DCM, the combined organic phases were dried over anhydrous sodium sulfate, filtered, and concentrated, the crude was purified by prep-HPLC to give the desired product SU20666-0108 (5.0 mg, yield: 2%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.65%, Rt=1.942 min; MS Calcd.: 273.0; MS Found: 274.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.274 min.

¹H NMR (400 MHz, CDCl₃) 4.72 (2H, s), 5.91 (1H, d, J=6.0 Hz), 6.48-6.53 (1H, m), 6.74-6.77 (2H, m), 6.99 (1H, d, J=3.2 Hz), 7.31 (1H, d, J=8.8 Hz), 8.48 (1H, s).

The names SU20666-0110, SP 110, and 110 all refer to the same compound having the formula:

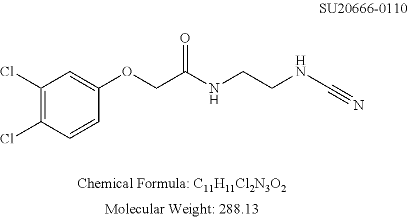 [SMILES: N#CNCCNC(=O)COc1ccc(Cl)c(Cl)c1]

Route for SU20666-0110

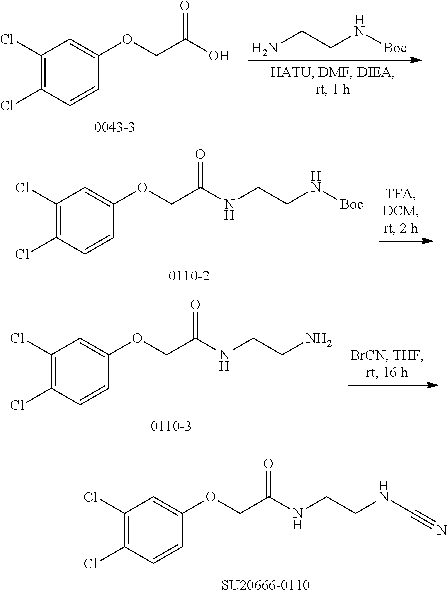 [SMILES: O=C(O)COc1ccc(Cl)c(Cl)c1 ; CC(C)(C)OC(=O)NCCN ; CC(C)(C)OC(=O)NCCNC(=O)COc1ccc(Cl)c(Cl)c1 ; NCCNC(=O)COc1ccc(Cl)c(Cl)c1 ; N#CNCCNC(=O)COc1ccc(Cl)c(Cl)c1]

The synthesis of tert-butyl (2-(2-(3,4-dichlorophenoxy)acetamido)ethyl)carbamate (110-2).

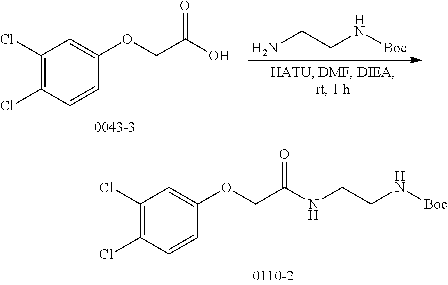 [SMILES: O=C(O)COc1ccc(Cl)c(Cl)c1 ; CC(C)(C)OC(=O)NCCN ; CC(C)(C)OC(=O)NCCNC(=O)COc1ccc(Cl)c(Cl)c1]

To a solution of compound 0043-3 (300 mg, 1.4 mmol) in DMF (10 mL) was added tert-butyl (2-aminoethyl)carbamate (260 mg, 1.6 mmol), DIEA (350 mg, 2.7 mmol) and HATU (800 mg, 2.1 mmol). The resulting reaction mixture was stirred for 1 h at rt, then added water, the aqueous phase was extracted with EA, the combined organic phases were dried over anhydrous sodium sulfate, filtered, and concentrated, the crude was purified by prep-HPLC to give the desired product 110-2 (280 mg, yield: 56%) as a yellow solid.

The synthesis of N-(2-aminoethyl)-2-(3,4-dichlorophenoxy)acetamide (110-3).

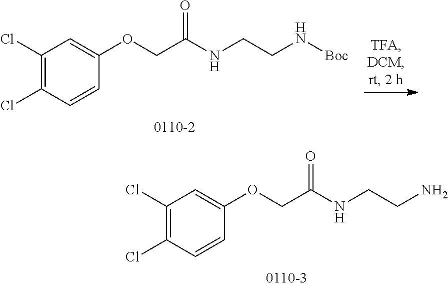 [SMILES: CC(C)(C)OC(=O)NCCNC(=O)COc1ccc(Cl)c(Cl)c1 ; NCCNC(=O)COc1ccc(Cl)c(Cl)c1]

To a stirred solution of compound 0110-2 (280 mg, 0.77 mmol) in DCM (10 mL) was added TFA (5 mL) at rt. The resulting reaction mixture was further stirred for 2 h at rt, then concentrated in vacuo to give the desired product 110-3 (220 mg, yield: 99%) as yellow oil.

The synthesis of N-(2-cyanamidoethyl)-2-(3,4-dichlorophenoxy)acetamide (SU20666-0110).

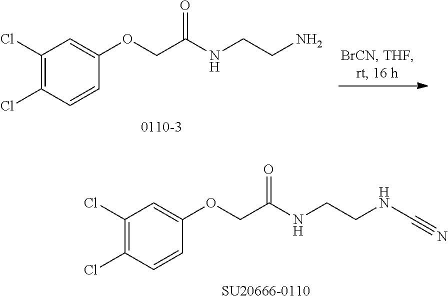 [SMILES: NCCNC(=O)COc1ccc(Cl)c(Cl)c1 ; N#CNCCNC(=O)COc1ccc(Cl)c(Cl)c1]

To a stirred solution of compound 0110-3 (100 mg, 0.38 mmol) in THF (10 mL) was added cyanic bromide (80 mg, 0.76 mmol) at rt. The resulting reaction mixture was further stirred for 2 h at rt, then concentrated in vacuo and purified by prep-HPLC to give the desired product SU20666-0110 (21 mg, yield: 19%) as a white solid.

LC-MS (Agilent LCMS 1200-6120, Column: Waters X-Bridge C18 (50 mm*4.6 mm*3.5μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.72%, Rt=1.709 min; MS Calcd.: 287.0; MS Found: 288.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.18%, Rt=8.148 min.

¹H NMR (400 MHz, DMSO-d₆) 3.01 (2H, t, J=6.0 Hz), 3.23-3.27 (2H, m), 4.54 (2H, s), 7.00 (1H, dd, J=8.8, 2.8 Hz), 7.28 (1H, d, J=2.8 Hz), 7.55 (1H, d, J=8.8 Hz), 8.27 (1H, s).

SU-20666-0111

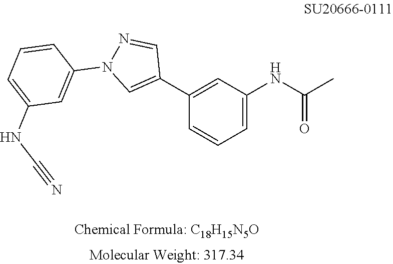 [SMILES: CC(=O)Nc1cccc(-c2cnn(-c3cccc(NC#N)c3)c2)c1]

Route for SU20666-0111

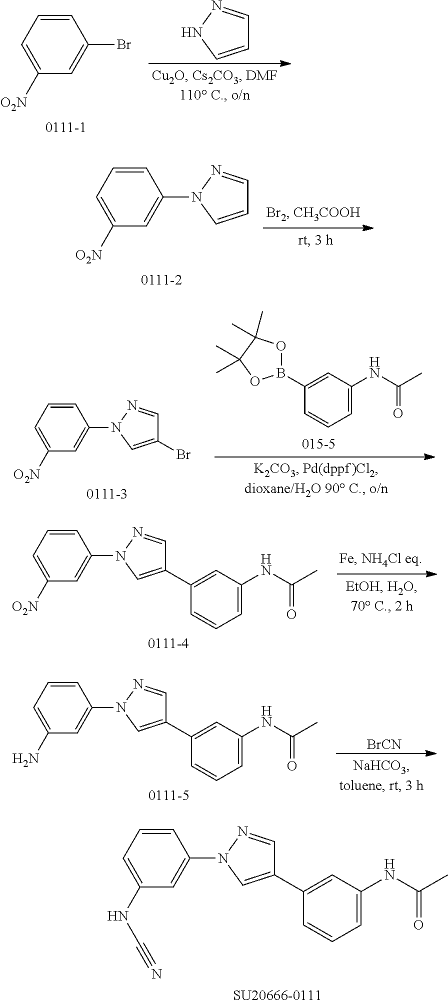

The synthesis of 1-(3-nitrophenyl)-1H-pyrazole (0111-2).

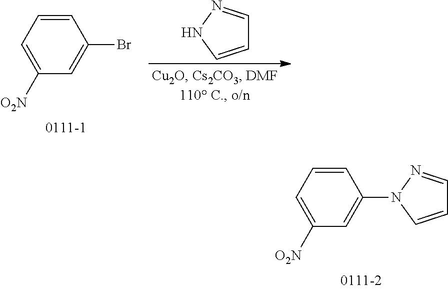 [SMILES: O=[N+]([O-])c1cccc(Br)c1 ; c1cn[nH]c1 ; O=[N+]([O-])c1cccc(-n2cccn2)c1]

To a solution of 0111-1 (4 g, 19.9 mmol) in DMF (30 mL) was added 1H-pyrazole (1.35 g, 19.9 mmol), Cu2O (285 mg, 1.99 mmol) and Cs₂CO₃ (19.5 g, 59.7 mmol). The mixture was stirred at 110° C. for o/n, then concentrated in vacuo to give crude compound, which was purified by pre-HPLC to afford 0111-2 (1.2 g, 32%) as a yellow solid.

The synthesis of 4-bromo-1-(3-nitrophenyl)-1H-pyrazole (0111-3).

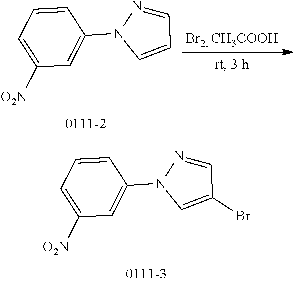

To a solution of 0111-2 (1.2 g, 6.3 mmol) in HOAc (15 mL) was added Br₂ (1.1 g, 6.9 mmol). The mixture was stirred at rt for 3 h, then concentrated in vacuo to give compound 0111-3 (450 mg, 27%) as a yellow solid.

The synthesis of N-(3-(1-(3-nitrophenyl)-1H-pyrazol-4-yl)phenyl)acetamide (0111-4).

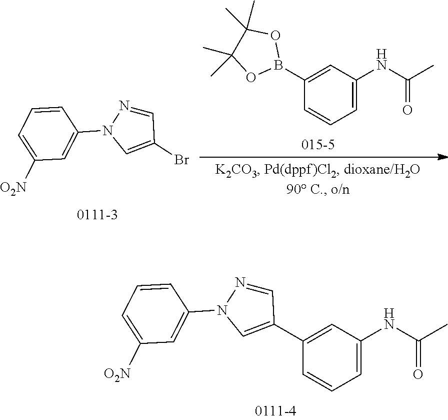 [SMILES: O=[N+]([O-])c1cccc(-n2cc(Br)cn2)c1 ; CC(=O)Nc1cccc(B2OC(C)(C)C(C)(C)O2)c1 ; CC(=O)Nc1cccc(-c2cnn(-c3cccc([N+](=O)[O-])c3)c2)c1]

To a solution of 0111-3 (450 mg, 1.7 mmol) in dioxane/H₂O (10/1 mL) was added 0115-5 (443 mg, 1.7 mmol), Pd(dppf)Cl₂(125 mg, 0.17 mmol) and K₂CO₃ (703 mg, 5.1 mmol). The resulting reaction mixture was heated to 90° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by C.C. to give the desired product 0111-4 (450 mg, 82%) as a yellow solid.

The synthesis of N-(3-(1-(3-aminophenyl)-1H-pyrazol-4-yl)phenyl)acetamide (0111-5).

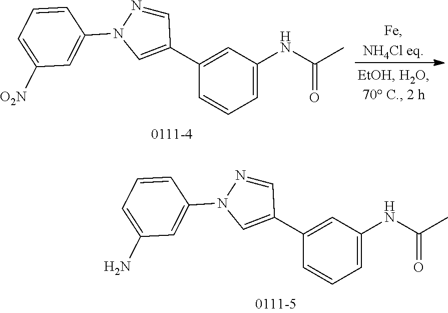 [SMILES: CC(=O)Nc1cccc(-c2cnn(-c3cccc([N+](=O)[O-])c3)c2)c1 ; "Fe,NH4Cl eq." ; CC(=O)Nc1cccc(-c2cnn(-c3cccc(N)c3)c2)c1]

To a solution of 0111-4 (450 mg, 1.4 mmol) in EtOH/H₂O (10/2 mL) was added Fe (7.8 mg, 0.14 mmol) and NH₄Cl (7.4 mg, 0.14 mmol). The mixture was stirred at 70° C. for 2 h. Then added water, the aqueous phase was extracted with ethyl acetate, the combined organic phases were dried over anhydrous sodium sulfate, filtered, and concentrated in vacuo and purified by prep-TLC to give the desired product 0111-5 (250 mg, 61%) as a yellow solid.

The synthesis of N-(3-(1-(3-cyanamidophenyl)-1H-pyrazol-4-yl)phenyl)acetamide (SU20666-0111).

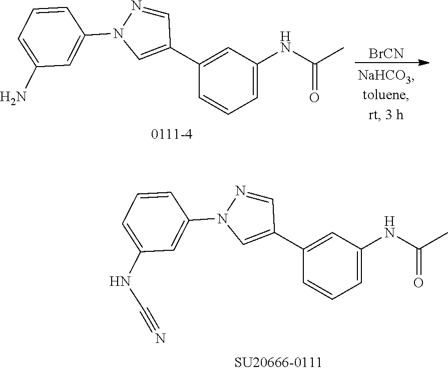

To a solution of 0111-5 (250 mg, 0.85 mmol) in toluene (5 mL) was added BrCN (90 mg, 0.85 mmol) and NaHCO₃ (214 mg, 2.55 mmol). The mixture was stirred at rt for 3 h, then added water, the aqueous phase was extracted with ethyl acetate, the combined organic phases were dried over anhydrous sodium sulfate, filtered, and concentrated in vacuo and purified by prep-HPLC to give the desired product SU20666-0111 (41 mg, 15%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 98.28%, Rt=1.539 min; MS Calcd.: 317.1; MS Found: 318.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 97.98%, Rt=7.607 min.

¹H NMR (400 MHz, DMSO-d₆) δ 2.06 (3H, s), 6.91 (1H, dd, J=7.9, 1.6 Hz), 7.33-7.60 (m, 6H), 7.85 (1H, s), 8.10 (1H, s), 8.92 (1H, s), 9.98 (1H, s), 10.46 (1H, s).

SU-20666-0112

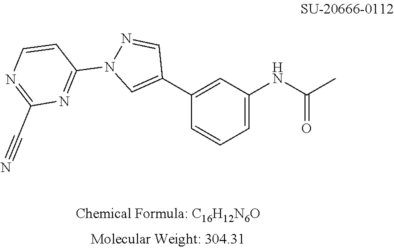 [SMILES: CC(=O)Nc1cccc(-c2cnn(-c3ccnc(C#N)n3)c2)c1]

Route for SU20666-0112

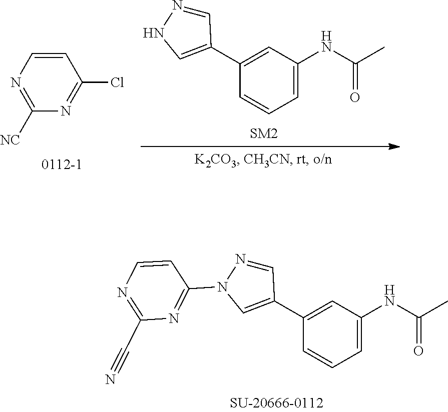 [SMILES: N#Cc1nccc(Cl)n1 ; CC(=O)Nc1cccc(-c2cn[nH]c2)c1 ; CC(=O)Nc1cccc(-c2cnn(-c3ccnc(C#N)n3)c2)c1]

The synthesis of N-(3-(1-(2-cyanopyrimidin-4-yl)-1H-pyrazol-4-yl)phenyl)acetamide (SU20666-0112).

To a solution of 0112-1 (50 mg, 0.36 mmol) in CH₃CN (5 mL) was added SM2 (73 mg, 0.36 mmol) and K₂CO₃ (150 mg, 1.08 mmol). The mixture was stirred at rt for o/n, then added water, the aqueous phase was extracted with dichloromethane, the combined organic phases were dried over anhydrous sodium sulfate, filtered, and concentrated, the crude was purified by prep-HPLC to afford SU20666-0112 (48 mg, 44%) as a white solid.

LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity: 99.35%, Rt=1.788 min; MS Calcd.:304.1; MS Found: 305.2 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity is 98.63%, Rt=8.566 min.

¹H NMR (400 MHz, DMSO-d₆) δ 2.07 (3H, s), 7.36 (1H, t, J=7.9 Hz), 7.52 (2H, t, J=8.9 Hz), 7.95 (1H, s), 8.21 (1H, d, J=5.7 Hz), 8.44 (1H, s), 8.97-9.12 (2H, m), 10.00 (1H, s).

SU20666-0113

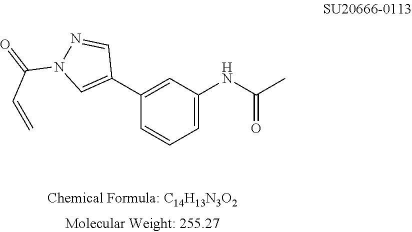 [SMILES: C=CC(=O)n1cc(-c2cccc(NC(C)=O)c2)cn1]

Route for SU20666-0113

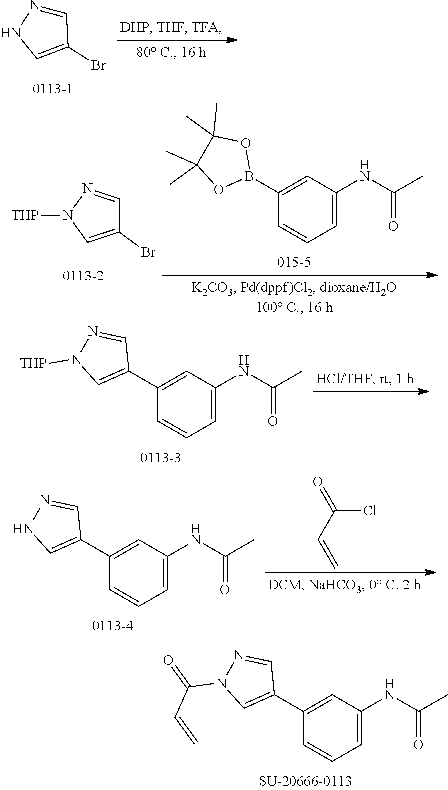

The synthesis of 4-bromo-1-(tetrahydro-2H-pyran-2-yl)-1H-pyrazole (0113-2).

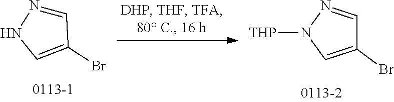

To a solution of compound 0113-1 (1.0 g, 6.8 mmol) in THF (20 mL) was added DTP (857 mg, 10.2 mmol) and TFA (catalytic amount). The resulting reaction mixture was heated to 80° C. and stirred for 16 h, then added water, the aqueous phase was extracted with EA, the combined organic phases were dried over anhydrous sodium sulfate, filtered, and concentrated, the crude was purified by prep-HPLC to give the desired product 113-2 (760 mg, yield: 48%) as yellow oil.

The synthesis of N-(3-(1-(tetrahydro-2H-pyran-2-yl)-1H-pyrazol-4-yl)phenyl)acetamide (0113-3).

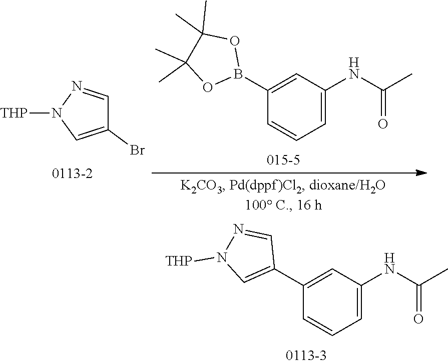

To a stirred solution of compound 0113-2 (760 mg, 3.3 mmol) in dioxane/water (20 mL/2 mL) was added 0015-5 (862 mg, 3.3 mmol), K₂CO₃ (911 mg, 6.6 mmol), Pd(dppf)Cl₂ (100 mg). The resulting reaction mixture was heated to 100° C. and stirred for 16 h and concentrated in vacuo to remove the solvent, then added water, the aqueous phase was extracted with dichloromethane, the combined organic phases were dried over anhydrous sodium sulfate, filtered, and concentrated, the crude was purified by prep-HPLC to give the desired product 0113-3 (510 mg, yield: 54%) as a yellow solid.

The synthesis of N-(3-(1H-pyrazol-4-yl)phenyl)acetamide (0113-4).

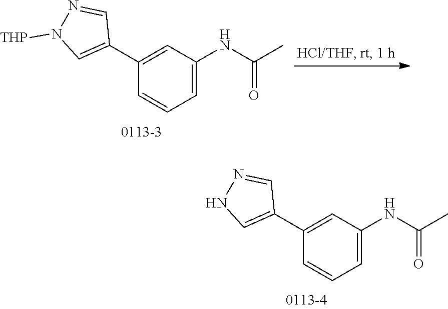

To a stirred solution of compound 0113-3 (510 mg, 1.8 mmol) in THF (10 mL) was added HCl (1.0 N, 2 mL) at rt. The resulting reaction mixture was further stirred for 1 h at rt, then concentrated in vacuo and purified by prep-HPLC to give the desired product 0113-4 (310 mg, yield: 86%) as a white solid.

The synthesis of N-(3-(1-acryloyl-1H-pyrazol-4-yl)phenyl)acetamide (SU20666-0113).

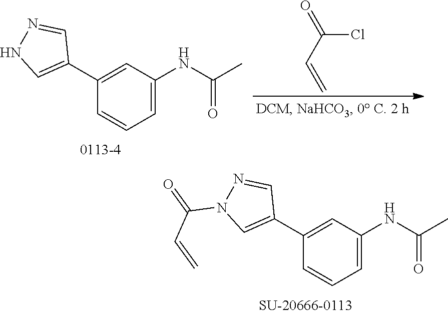

To a solution of compound 0113-4 (100 mg, 0.50 mmol) in DCM (6 mL) was added NaHCO₃ (84 mg, 1.0 mmol) and acryloyl chloride (45 mg, 0.50 mmol) at 0° C. and stirred at this temperature for 2 min, then added water, the aqueous phase was extracted with DCM, the combined organic phases were dried over anhydrous sodium sulfate, filtered, and concentrated, the crude was purified by prep-HPLC to give the desired product SU20666-0113 (31 mg, yield: 24%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.579 min; MS Calcd.: 255.1; MS Found: 256.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=7.872 min.

¹H NMR (400 MHz, CDCl₃) δ 2,14 (3H, s), 6.04 (1H, dd, J=10.8, 2.0 Hz), 6.70 (1H, dd, J=17.2, 1.6 Hz), 7.15 (1H, s), 7.22-7.31 (3H, m), 7.47-7.54 (1H, m), 7.75 (1H, s), 7.96 (1H, s), 8.49 (1H, s).

SU20666-0116

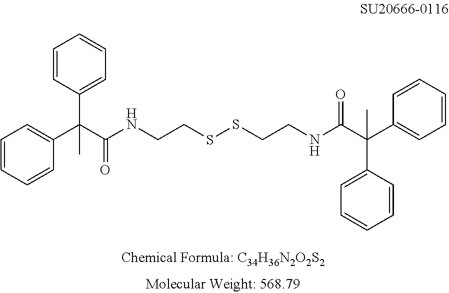 [SMILES: CC(C(=O)NCCSSCCNC(=O)C(C)(c1ccccc1)c1ccccc1)(c1ccccc1)c1ccccc1]

Route for SU20666-0116

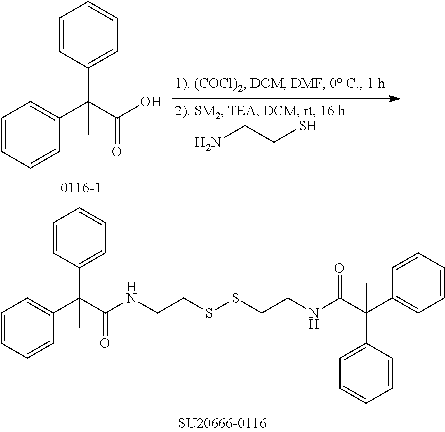

The synthesis of N,N′-(disulfanediylbis(ethane-2,1-diyl))bis(2,2-diphenylpropanamide) (SU20666-0116).

To a stirred solution of 0116-1 (100 mg, 0.44 mmol) in dichloromethane (10 ml) was added oxalyl chloride (280 mg, 2.2 mmol) and DMF (0.05 mL) at 0° C. The resulting reaction mixture was stirred at 0° C. for 1 h and concentrated in vacuo, the crude was dissolved in dichloromethane (10 mL), was added TEA (220 mg, 2.2 mmol) and 2-aminoethanethiol (59 mg, 0.88 mmol), then the reaction mixture was stirred for another 16 h at rt. Water (10 mL) was added, the aqueous phase was extracted with dichloromethane, the combined organic phases were dried over anhydrous sodium sulfate, filtered, and concentrated in vacuopurified by prep-HPLC to give the desired product SU20666-0116 (30 mg, yield: 24%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 95.84%, Rt=2.147 min; MS Calcd.: 568.2; MS Found: 569.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 86.46%, Rt=11.283 min.

¹H NMR (400 MHz, CDCl₃) δ 1.59 (6H, s), 2.72 (4H, t, J=6.4 Hz), 3.52 (4H, q, J=6.4 Hz), 5.89 (2H, t, J=6.4 Hz), 7.23-7.36 (19H, m).

The names SU20666-0117, SP 117, and 117 all refer to the same compound having the formula:

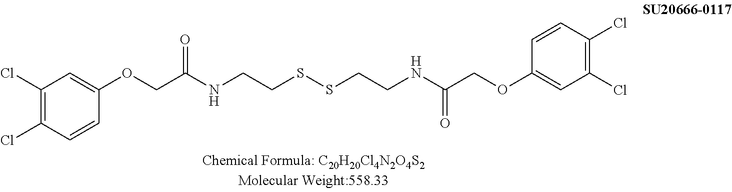

Route for SU20666-0117

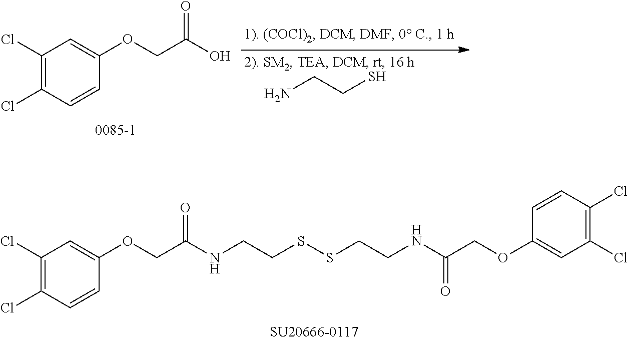

The synthesis of N,N′-(disulfanediylbis(ethane-2,1-diyl))bis(2-(3,4-dichlorophenoxy)acetamide) (SU20666-0117).

To a stirred solution of 0085-1 (200 mg, 0.92 mmol) in dichloromethane (10 ml) was added oxalyl chloride (0.56 g, 4.6 mmol) and DMF (0.05 mL) at 0° C. The resulting reaction mixture was stirred at 0° C. for 1 h and concentrated in vacuo, the crude was dissolved in dichloromethane (10 mL), was added TEA (533 mg, 5.28 mmol) and 2-aminoethanethiol (136 mg, 1.76 mmol), then the reaction mixture was stirred for another 16 h at rt. Water (10 mL) was added, the aqueous phase was extracted with dichloromethane, the combined organic phases were dried over anhydrous sodium sulfate, filtered, and concentrated in vacuopurified by prep-HPLC to give the desired product SU20666-0117 (30 mg, yield: 12%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=2.035 min; MS Calcd.: 556.0; MS Found: 557.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 94.68%, Rt=10.732 min.

¹H NMR (400 MHz, DMSO-d₆) δ 2.82 (4H, t, J=6.8 Hz), 3.42 (4H, q, J=6.4 Hz), 4.54 (4H, s), 6.99 (2H, dd, J=8.8, 2.8 Hz), 7.25 (2H, d, J=2.8 Hz), 7.54 (2H, d, J=8.8 Hz), 8.31 (2H, t, J=6.0 Hz).

The names SU20666-0119, SP 119, and 119 all refer to the same compound having the formula:

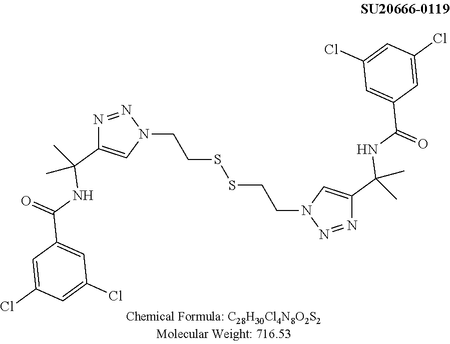 [SMILES: CC(C)(NC(=O)c1cc(Cl)cc(Cl)c1)c1cn(CCSSCCn2cc(C(C)(C)NC(=O)c3cc(Cl)cc(Cl)c3)nn2)nn1]

Route for SU20666-0119

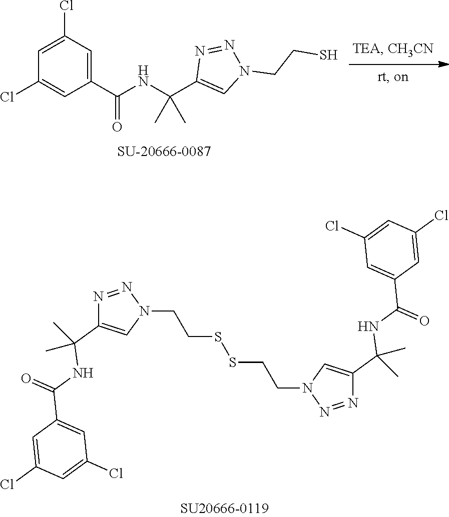 [SMILES: CC(C)(NC(=O)c1cc(Cl)cc(Cl)c1)c1cn(CCS)nn1 ; CC(C)(NC(=O)c1cc(Cl)cc(Cl)c1)c1cn(CCSSCCn2cc(C(C)(C)NC(=O)c3cc(Cl)cc(Cl)c3)nn2)nn1]

The synthesis of N,N′-(2,2′-(1,1′-(2,2′-disulfanediylbis(ethane-2,1-diyl))bis(1H-1,2,3-triazole-4,1-diyl))bis(propane-2,2-diyl))bis(3,5-dichlorobenzamide) (SU20666-0119).

To a stirred solution of compound SU20666-0987 (40 mg, 0.11 mmol) in CH₃CN was added TEA (44 mg, 0.33 mmol). The resulting reaction mixture was stirred at rt overnight. Then added water, the aqueous phase was extracted with dichloromethane, the combined organic phases were dried over anhydrous sodium sulfate, filtered, and concentrated in vacuopurified by prep-HPLC to give the desired product SU20666-0119 (16 mg, yield: 20.0%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.98%, Rt=2.275 min; MS Calcd.: 714.0; MS Found:715.1[M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.091 min.

¹H NMR (400 MHz, DMSO-d₆) δ 1.69 (12H, s), 2.23 (4H, t, J=6.8 Hz), 4.57 (4H, t, J=6.8 Hz), 7.77-7.83 (6H, m), 8.00 (2H, s), 8.60 (2H, s).

The names SU20666-0123, SU20666-0123-01, SP 123, and 123 all refer to the same compound having the formula:

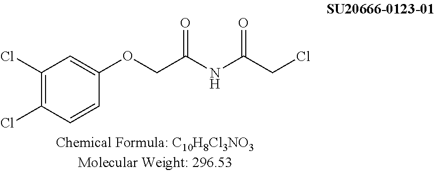 [SMILES: O=C(CCl)NC(=O)COc1ccc(Cl)c(Cl)c1]

Route for SU20666-0123

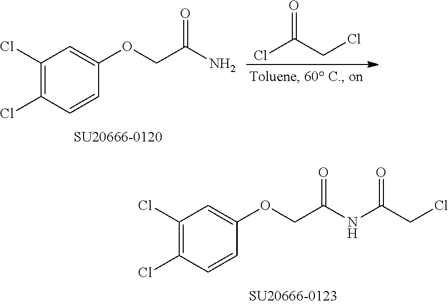 [SMILES: NC(=O)COc1ccc(Cl)c(Cl)c1 ; O=C(Cl)CCl ; O=C(CCl)NC(=O)COc1ccc(Cl)c(Cl)c1]

The synthesis of 2-chloro-N-(2-(3,4-dichlorophenoxy)acetyl)acetamide (SU20666-0123).

To a stirred solution of compound SU20666-0120 (200 mg, 0.91 mmol) in toluene (5 ml) was added 2-chloroacetyl chloride (0.2 ml, 1.36 mmol). The resulting reaction mixture was stirred at 60° C. overnight. Then added water, the aqueous phase was extracted with EA, the combined organic phases were dried over anhydrous sodium sulfate, filtered, and concentrated in vacuo, purified by prep-HPLC to give the desired product SU20666-0123 (20 mg, yield: 7.5%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 95.02%, Rt=1.818 min; MS Calcd.: 295.0; MS Found: 296.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 93.57%, Rt=9.211 min.

¹H NMR (400 MHz, DMSO-d₆) δ 4.46 (2H, s), 4.98 (2H, s), 6.95 (1H, dd, J=8.8 Hz, J=2.8 Hz), 7.23 (1H, d, J=2.8 Hz), 7.49 (1H, d, J=8.8 Hz), 11.27 (1H, s).

SU20666-0125

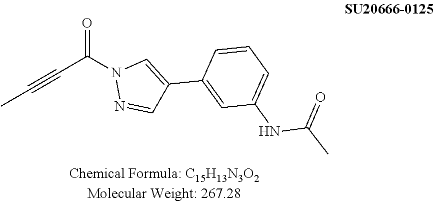 [SMILES: CC#CC(=O)n1cc(-c2cccc(NC(C)=O)c2)cn1]

Route for SU20666-0125

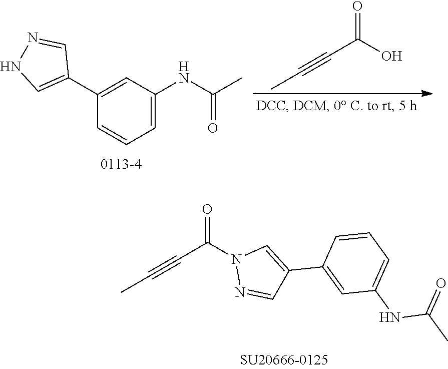

The synthesis of N-(3-(1-but-2-ynoyl-1H-pyrazol-4-yl)phenyl)acetamide (SU20666-0125).

To a solution of 0113-4 (250 mg, 1.24 mmol) in DCM (10 mL) was added but-2-ynoic acid (104 mg, 1.24 mmol) and DCC (30 mg, 0.15 mmol). The mixture was stirred at 0° C. to rt for 5 h. The mixture was concentrated in vacuo to give crude compound. The crude product was purified by pre-HPLC to afford SU20666-0125 (20 mg, 6%) as a white solid.

LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity: 100%, Rt=1.515 min; MS Calcd.: 267.1; MS Found: 268.2 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 86.75%, Rt=7.448 min.

¹H NMR (400 MHz, DMSO-d₆) δ 2.06 (3H, s), 2.24 (3H, s), 7.30-7.53 (3H, m), 7.88 (1H, s), 8.34 (1H, s), 8.79 (1H, s), 10.00 (1H, s).

SU20666-0126

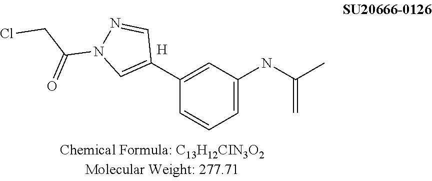

Route for SU20666-0126

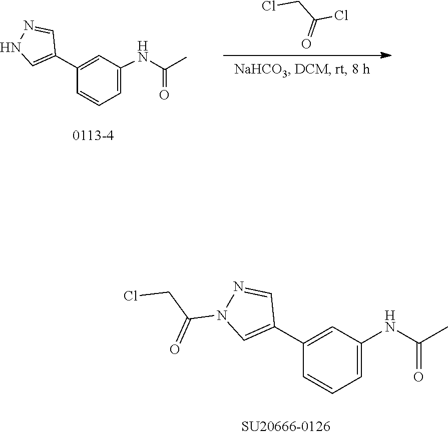 [SMILES: CC(=O)Nc1cccc(-c2cn[nH]c2)c1 ; O=C(Cl)CCl ; CC(=O)Nc1cccc(-c2cnn(C(=O)CCl)c2)c1]

The synthesis of N-(3-(1-(2-chloroacetyl)-1H-pyrazol-4-yl)phenyl)acetamide (SU20666-0126).

To a stirred solution of compound 0113-4 (200 mg, 1.09 mmol) in DCM (5 ml) was added 2-chloroacetyl chloride (0.25 ml, 1.64 mmol). The resulting reaction mixture was stirred at rt for 8 h. Then added water, the aqueous phase was extracted with DCM, the combined organic phases were dried over anhydrous sodium sulfate, filtered, and concentrated in vacuo, purified by prep-HPLC to give the desired product SU20666-0126 (50 mg, yield: 18.1%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 89.23%, Rt=1.593 min; MS Calcd.: 277.0; MS Found: 278.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 89.78%, Rt=7.921 min.

¹H NMR (400 MHz, DMSO-d₆) δ 2.06 (3H, s), 5.21 (2H, s), 7.35 (1H, t, J=8.0 Hz), 7.45-7.52 (2H, m), 7.88 (1H, s), 8.33 (1H, s), 8.80 (1H, s), 10.0 (1H, s).

SU20666-0130

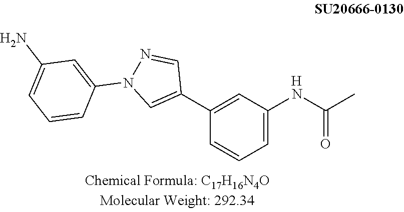

Route for SU20666-0130

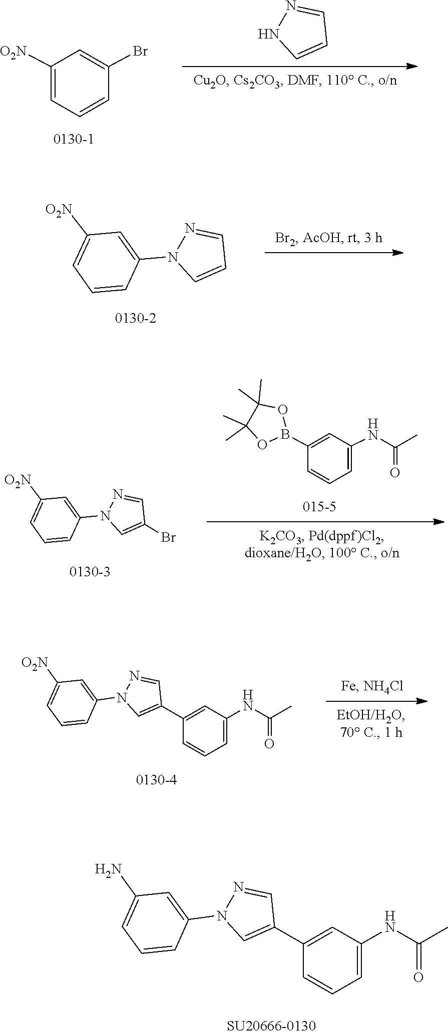

The synthesis of 1-(3-nitrophenyl)-1H-pyrazole (0130-2).

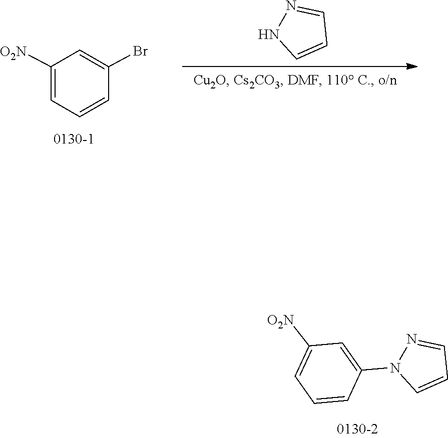 [SMILES: O=[N+]([O-])c1cccc(Br)c1 ; c1cn[nH]c1 ; O=[N+]([O-])c1cccc(-n2cccn2)c1]

To a solution of 1-bromo-3-nitrobenzene (4 g, 19.8 mmol) in DMF (15 mL) was added 1H-pyrazole (898 mg, 13.2 mmol), Cu2O (0.2 g, 1.2 mmol) and Cs₂CO₃ (7.8 g, 23.8 mmol). The mixture was stirred at 110° C. for o/n, then concentrated in vacuo to give crude compound, which was further purified by pre-HPLC to afford compound 0130-2 (1.2 g, 32%) as a yellow solid.

The synthesis of 4-bromo-1-(3-nitrophenyl)-1H-pyrazole (0130-3).

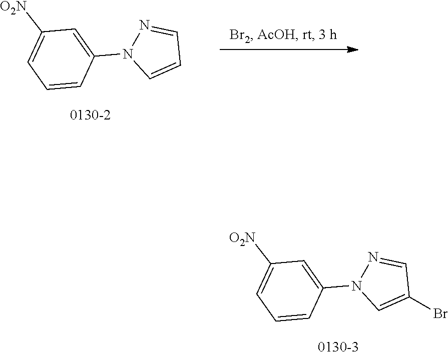 [SMILES: O=[N+]([O-])c1cccc(-n2cccn2)c1 ; O=[N+]([O-])c1cccc(-n2cc(Br)cn2)c1]

To a solution of 0130-2 (1.2 g, 6.3 mmol) in AcOH (10 mL) was added Br₂ (3 mL, 6.9 mmol). The mixture was stirred at rt for 3 h, then added water, the aqueous phase was extracted with dichloromethane, the combined organic phases were dried over anhydrous sodium sulfate, filtered, and concentrated to give the desired product 0130-3 (765 mg, 45%) as a yellow solid.

The synthesis of N-(3-(1-(3-nitrophenyl)-1H-pyrazol-4-yl)phenyl)acetamide (0130-4).

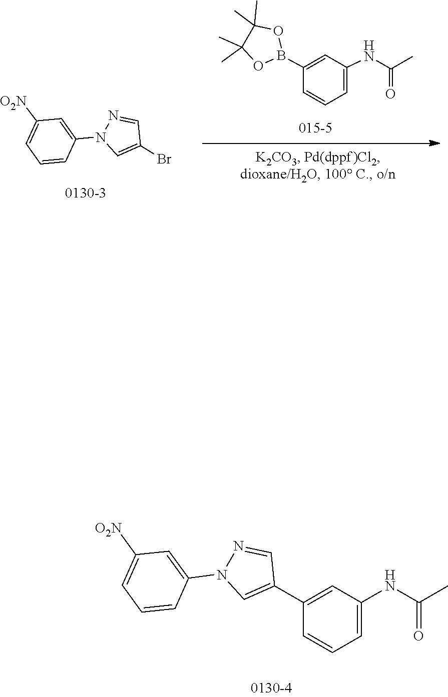 [SMILES: O=[N+]([O-])c1cccc(-n2cc(Br)cn2)c1 ; CC(=O)Nc1cccc(B2OC(C)(C)C(C)(C)O2)c1 ; CC(=O)Nc1cccc(-c2cnn(-c3cccc([N+](=O)[O-])c3)c2)c1]

To a stirred solution of compound 0130-3 (765 mg, 2.8 mmol) in dioxane/water (10 mL/2 mL) was added 015-5 (1.1 g, 4.2 mmol), K₂CO₃ (1.2 g, 8.4 mmol), Pd(dppf)Cl₂ (220 mg, 0.3 mmol). The resulting reaction mixture was heated to 100° C. and stirred for 16 h and concentrated in vacuo to remove the solvent, then added water, the aqueous phase was extracted with dichloromethane, the combined organic phases was dried over anhydrous sodium sulfate, filtered, and concentrated, the crude was purified by prep-HPLC to give the desired product 0130-4 (495 mg, 55%) as a yellow solid.

The synthesis of N-(3-(1-(3-aminophenyl)-1H-pyrazol-4-yl)phenyl)acetamide (SU20666-0130).

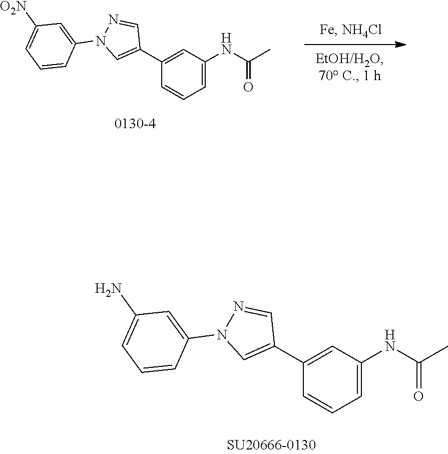 [SMILES: CC(=O)Nc1cccc(-c2cnn(-c3cccc([N+](=O)[O-])c3)c2)c1 ; CC(=O)Nc1cccc(-c2cnn(-c3cccc(N)c3)c2)c1]

To a stirred solution of 0130-4 (400 mg, 1.24 mmol) in EtOH/H₂O (10 mL/1 mL) was added Fe powder (69 mg, 12.4 mmol) and NH₄Cl (66 mg, 12.4 mmol) at rt. The resulting reaction mixture was stirred for 1 h at 70° C. Then added water, the aqueous phase was extracted with ethyl acetate, the combined organic phases were dried over anhydrous sodium sulfate, filtered, and concentrated in vacuo and purified by prep-HPLC to give the desired product SU20666-0130 (36 mg, 10%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.368 min; MS Calcd.: 292.1; MS Found: 293.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=5.847 min.

¹H NMR (400 MHz, DMSO-d₆) δ 2.06 (3H, s), 6.75 (2H, d, J=3.7 Hz), 7.12-7.62 (7H, m), 7.87 (1H, s), 8.06 (1H, s), 8.78 (1H, s), 9.98 (1H, s).

The names SU20666-0131, SP 131, and 131 all refer to the same compound having the formula as shown below.

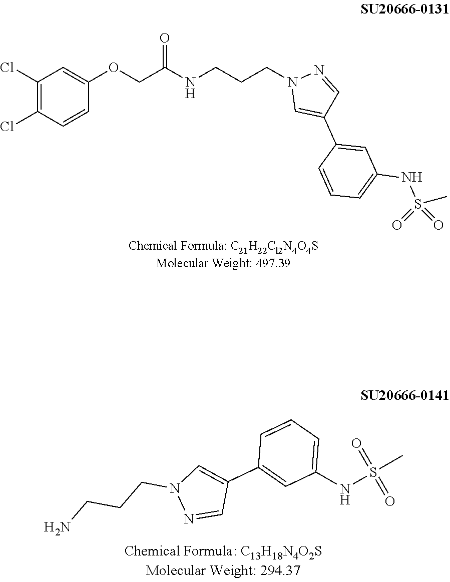 [SMILES: CS(=O)(=O)Nc1cccc(-c2cnn(CCCNC(=O)COc3ccc(Cl)c(Cl)c3)c2)c1 ; CS(=O)(=O)Nc1cccc(-c2cnn(CCCN)c2)c1]

Route for SU20666-0131 and SU20666-0141

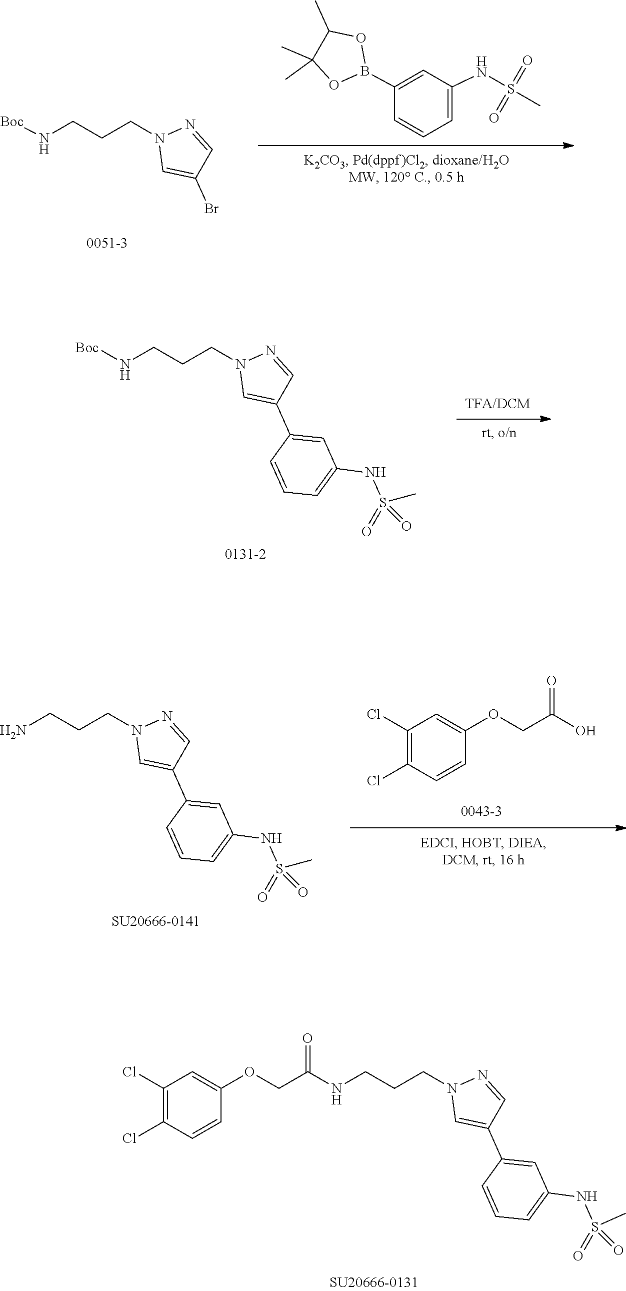

The synthesis of tert-butyl 3-(4-(3-(methylsulfonamido)phenyl)-1H-pyrazol-1-yl)propylcarbamate (0131-2).

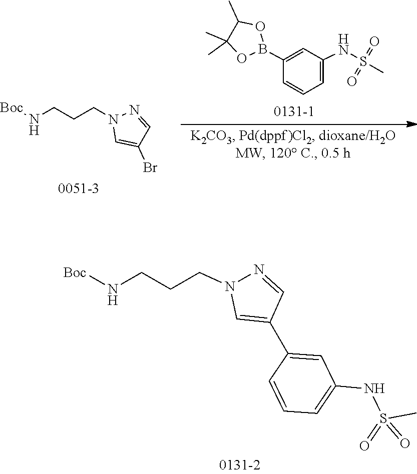

To a stirred solution of compound 0051-3 (2.0 g, 6.6 mmol) in dioxane/water (20 mL/2 mL) was added 0131-1 (1.87 g, 6.6 mmol), K₂CO₃ (2.73 g, 19.8 mmol), Pd(dppf)Cl₂ (483 mg, 0.66 mmol). The resulting reaction mixture was heated to 120° C. and stirred for 0.5 h under MW condition and concentrated in vacuo to remove the solvent, then added water, the aqueous phase was extracted with dichloromethane, the combined organic phases was dried over anhydrous sodium sulfate, filtered, and concentrated, the crude was purified by prep-HPLC to give the desired product 0131-2 (500 mg, 19%) as a yellow solid.

The synthesis of N-(3-(1-(3-aminopropyl)-1H-pyrazol-4-yl)phenyl)methanesulfonamide (SU20666-0141).

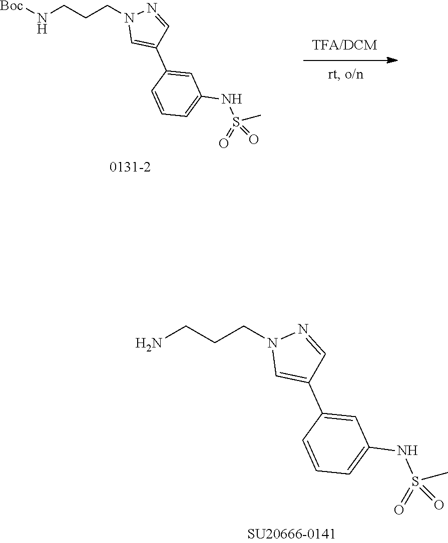 [SMILES: CC(C)(C)OC(=O)NCCCn1cc(-c2cccc(NS(C)(=O)=O)c2)cn1 ; CS(=O)(=O)Nc1cccc(-c2cnn(CCCN)c2)c1]

To a solution of 0131-2 (500 mg, 1.27 mmol) in DCM (10 mL) was added TFA (5 mL). The mixture was stirred at rt for o/n, then concentrated in vacuo and purified by prep-HPLC to give compound SU20666-0141 (360 mg, 96.5%) as a yellow solid.

LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity: 98.47%, Rt=1.226 min; MS Calcd.: 496.1; MS Found: 497.0 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=5.999 min.

¹H NMR (400 MHz, DMSO-d₆) δ 1.88 (2H, t, J=6.4 Hz), 2.88-2.90 (2H, m), 2.95 (3H, s), 4.11 (2H, t, J=6.4 Hz), 4.50 (2H, brs), 6.05-6.79 (1H, brs), 6.99 (1H, d, J=7.2 Hz), 7.22-7.29 (m, 3H), 7.76 (1H, s), 8.11 (1H, s).

The synthesis of 2-(3,4-dichlorophenoxy)-N-(3-(4-(3-(methylsulfonamido) phenyl)-1H-pyrazol-1-yl)propyl)acetamide (SU20666-0131).

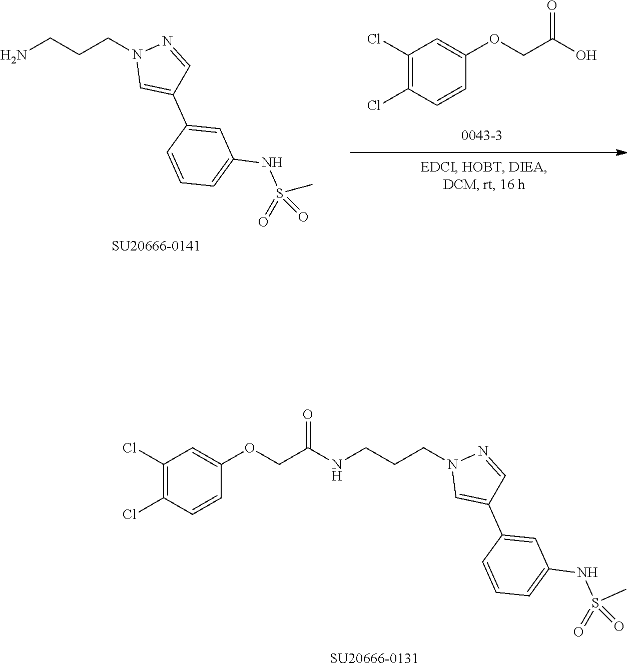 [SMILES: CS(=O)(=O)Nc1cccc(-c2cnn(CCCN)c2)c1 ; O=C(O)COc1ccc(Cl)c(Cl)c1 ; CS(=O)(=O)Nc1cccc(-c2cnn(CCCNC(=O)COc3ccc(Cl)c(Cl)c3)c2)c1]

To a solution of compound SU20666-0141 (360 mg, 1.22 mmol) in DCM (10 mL) was added 0043-3 (268 mg, 1.22 mmol), DIEA (472 mg, 3.66 mmol), EDCI (234 mg, 1.22 mmol) and HOBT (165 mg, 1.22 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0131 (32 mg, 5%) as a white solid.

LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity: 98.81%, Rt=1.838 min; MS Calcd.: 496.1; MS Found: 497.0 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 97.06%, Rt=9.172 min.

¹H NMR (400 MHz, DMSO-d₆) δ 1.93-2.00 (2H, m), 3.00 (3H, s), 3.14 (2H, q, J=6.8 Hz), 4.13 (2H, t, J=6.8 Hz), 4.55 (2H, s), 6.98-7.05 (2H, m), 7.26-7.34 (m, 4H), 7.55 (1H, d, J=8.8 Hz), 7.80 (1H, s), 8.12 (1H, s), 8.22 (1H , t, J=5.6 Hz), 9.72 (s, 1H).

The names SU20666-0133, SP 133, and 133 all refer to the same compound having the formula:

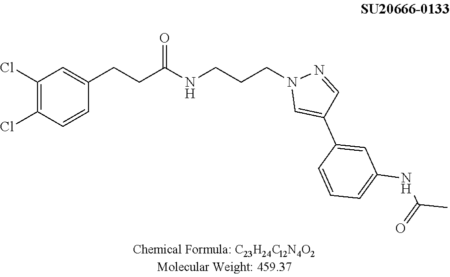 [SMILES: CC(=O)Nc1cccc(-c2cnn(CCCNC(=O)CCc3ccc(Cl)c(Cl)c3)c2)c1]

Route for SU20666-0133

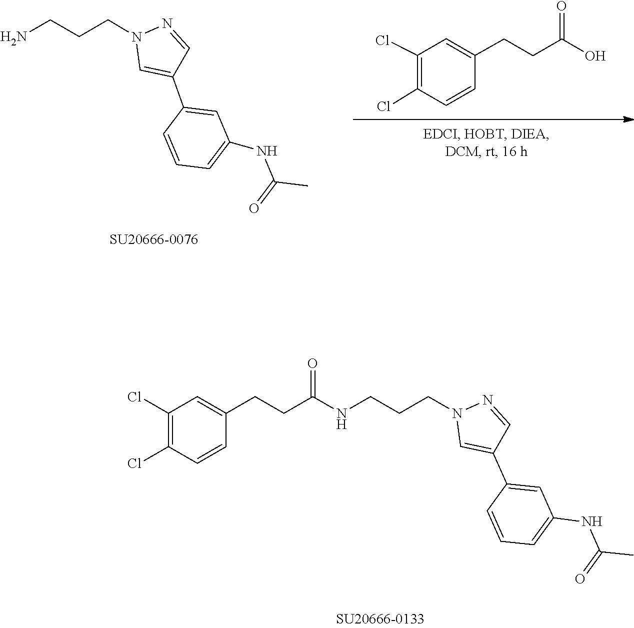 [SMILES: CC(=O)Nc1cccc(-c2cnn(CCCN)c2)c1 ; O=C(O)CCc1ccc(Cl)c(Cl)c1 ; CC(=O)Nc1cccc(-c2cnn(CCCNC(=O)CCc3ccc(Cl)c(Cl)c3)c2)c1]

The synthesis of 2-(3,4-dichlorophenoxy)-N-(3-(4-(3-(methylsulfonamido) phenyl)-1H-pyrazol-1-yl)propyl)acetamide (SU20666-0133).

To a solution of compound SU20666-0076 (80 mg, 0.31 mmol) in DCM (10 mL) was added3-(3,4-dichlorophenyl)propanoic acid (68 mg, 0.31 mmol), DIEA (120 mg, 0.93 mmol), EDCI (59 mg, 0.31 mmol) and HOBT (42 mg, 1.22 mmol). The resulting reaction mixture was stirred for 16 h at rt, then added water, the aqueous phase was extracted with EA, the combined organic phases were dried over anhydrous sodium sulfate, filtered, and concentrated, the crude was purified by prep-HPLC to give the desired product SU20666-0133 (20 mg, 14%) as a white solid.

LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity: 99.38%, Rt=1.842 min; MS Calcd.: 458.1; MS Found: 459.2 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity is 99.23%, Rt=8.584 min.

¹H NMR (400 MHz, DMSO-d₆) δ 1.79-1.92 (2H, m), 2.02 (3H, s), 2.36 (2H, t, J=7.2 Hz), 2.80 (2H, t, J=7.2 Hz), 3.00 (2H, q, J=2.4 Hz), 4.04 (2H, t, J=6.4 Hz), 7.17-7.24 (3H, m), 7.35 (1H, d, J=8.0 Hz), 7.46-7.71 (2H, m), 7.73-7.74 (2H,m), 7.89 (1H, t, J=5.2 Hz), 8.02 (s, 1H), 9.91 (s, 1H).

The names SU20666-0134, SP 134, and 134 all refer to the same compound having the formula as shown below.

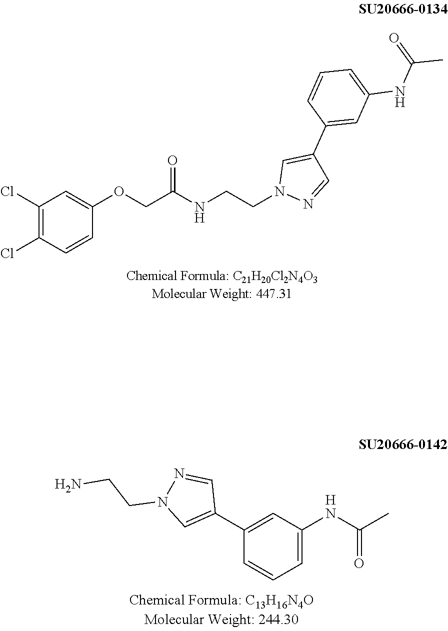

Route for SU20666-0134 and SU20666-0142

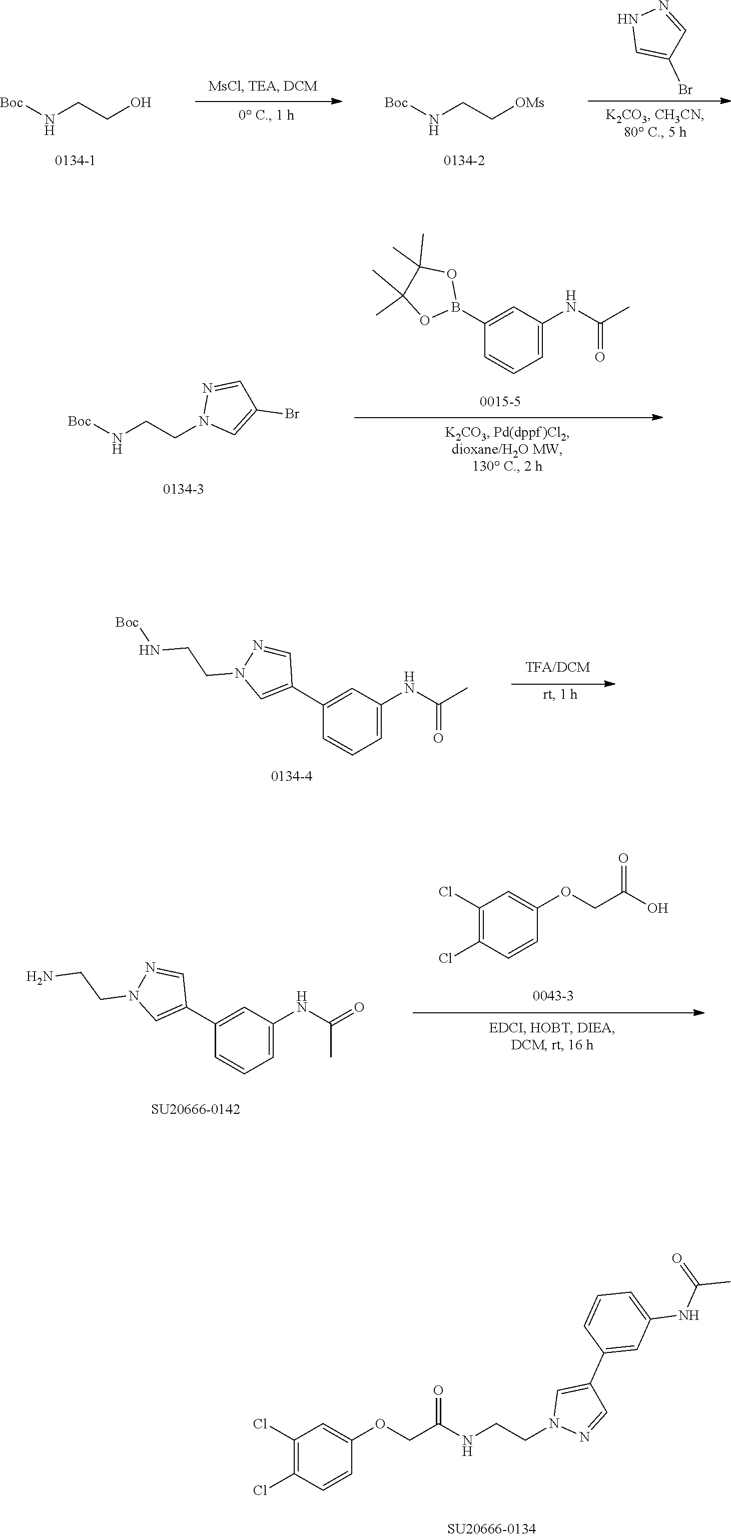

The synthesis of 2-(tert-butoxycarbonylamino)ethyl methanesulfonate (0134-2).

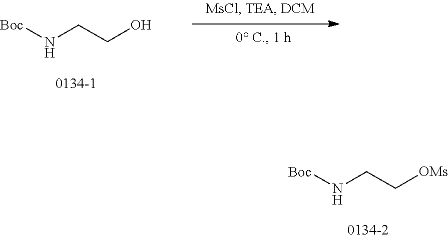 [SMILES: CC(C)(C)OC(=O)NCCO ; CC(C)(C)OC(=O)NCCOS(C)(=O)=O]

To a stirred solution of compound 0134-1 (2 g, 12.4 mmol) in DCM (20 ml) was added MsCl (2.13 g, 18.6 mmol) and TEA (4.8 g, 37.2 mmol) under ice-water. The resulting reaction mixture was stirred at 0° C. for 1 h. Then added water, the aqueous phase was extracted with DCM, the combined organic phases were dried over anhydrous sodium sulfate, filtered, and concentrated in vacuo to give the desired product 0134-2 (2 g, yield: 67.5%) as yellow oil.

The synthesis of tert-butyl 2-(4-bromo-1H-pyrazol-1-yl)ethylcarbamate (0134-3).

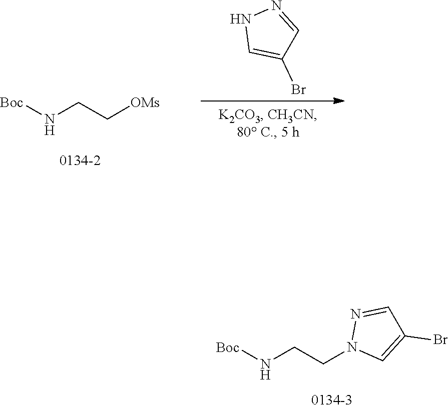 [SMILES: Brc1cn[nH]c1 ; CC(C)(C)OC(=O)NCCOS(C)(=O)=O ; CC(C)(C)OC(=O)NCCn1cc(Br)cn1]

To a solution of compound 0134-2 (2.0 g, 8.37 mmol) in CH₃CN (18 mL) was added 4-bromo-1H-pyrazole (0.95 g, 6.44 mmol) and K₂CO₃ (1.33 g, 9.66 mmol). The mixture was stirred at 80° C. for 5 h. The solvent was removed, added water, the aqueous phase was extracted with DCM, the combined organic phases were dried over anhydrous sodium sulfate, filtered, and concentrated in vacuo, purified by silical to give the desired product 0134-3 (1.0 g, yield: 41.2%) as a yellow solid.

The synthesis of tert-butyl 2-(4-(3-acetamidophenyl)-1H-pyrazol-1-yl)ethylcarbamate (0134-4).

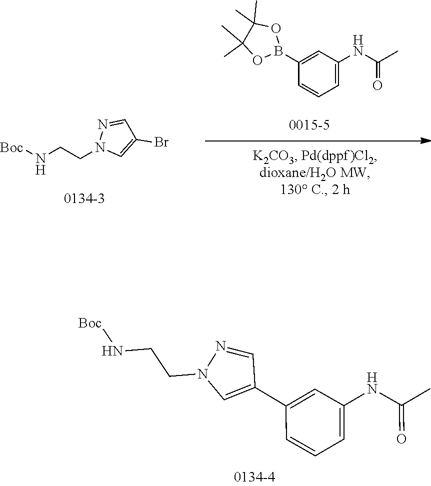 [SMILES: CC(=O)Nc1cccc(B2OC(C)(C)C(C)(C)O2)c1 ; CC(C)(C)OC(=O)NCCn1cc(Br)cn1 ; CC(=O)Nc1cccc(-c2cnn(CCNC(=O)OC(C)(C)C)c2)c1]

A solution of 0134-3 (1 g, 3.46 mmol) in dioxane/H₂O (15 ml/3 ml), was added 0015-5 (1.08 g, 4.2 mmol), K₂CO₃ (1.43 g, 10.4 mmol) and Pd(dppf)Cl₂ (0.1 g) under an argon atmosphere. The mixture was stirred at 130° C. for 2 h under microwave condition. After being cooled to room temperature, water was added. The aqueous phase was extracted with DCM (20 mL x 3), and the combined organic phases were dried over anhydrous sodium sulfate, filtered, and concentrated in vacuo. The crude product was purified to give compound 0134-4 (300 mg, 22.6%) as a yellow solid.

The synthesis of N-(3-(1-(2-aminoethyl)-1H-pyrazol-4-yl)phenyl)acetamide (SU20666-0142).

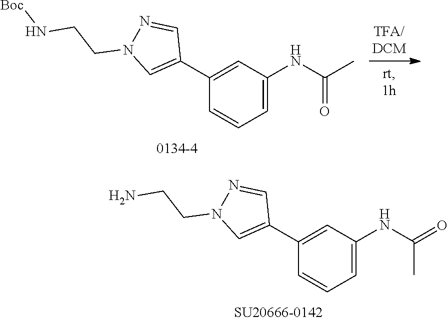 [SMILES: CC(=O)Nc1cccc(-c2cnn(CCNC(=O)OC(C)(C)C)c2)c1 ; CC(=O)Nc1cccc(-c2cnn(CCN)c2)c1]

To a stirred solution of compound 0134-4 (300 mg, 0.87 mmol) in DCM (5 ml) was added TFA (1.0 ml). The resulting reaction mixture was stirred at rt for 1 h. The solvent was removed, water (5 mL) was added to residue and the pH value was adjusted to ˜6.0 with NaHCO₃ (aq). The aqueous phase was extracted with dichloromethane, the combined organic phases were dried over anhydrous sodium sulfate, filtered, and concentrated in vacuo , purified by prep-HPLC to give SU20666-0142 (80 mg, yield: 38%) as colorless oil.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.34%, Rt=1.186 min; MS Calcd.: 244.1; MS Found: 245.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 99.49%, Rt=5.398 min.

¹H NMR (400 MHz, DMSO-d₆) δ 2.02 (3H, s), 2.91 (2H, t, J=6.4 Hz), 4.07 (2H, t, J=6.4 Hz), 7.19-7.26 (2H, m), 7.36 (1H, dd, J=6.0, 2.0 Hz), 7.72-7.75 (2H, m), 8.02-8.04 (1H, m), 9.91 (1H, s).

The synthesis of N-(2-(4-(3-acetamidophenyl)-1H-pyrazol-1-yl)ethyl)-2-(3,4-dichlorophenoxy)acetamide (SU20666-0134).

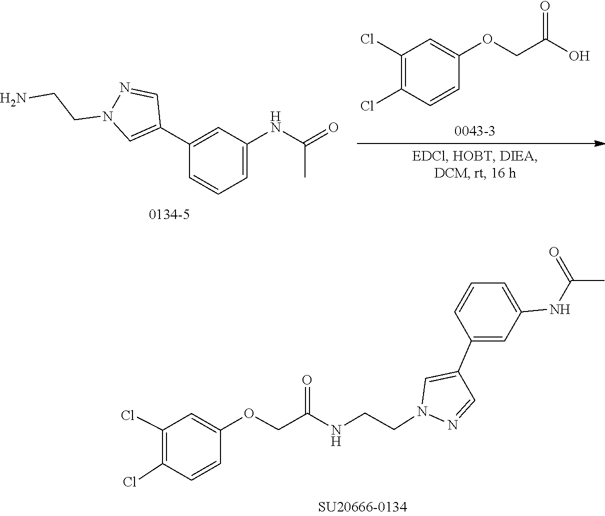

To a stirred solution of compound 0134-5 (50 mg, 0.2 mmol) in DCM (3 ml) was added 0043-3 (38 mg, 0.17 mmol), EDCI (49 mg, 0.26 mmol), HOBt (35 mg, 0.26 mmol) and DIEA (0.2 ml, 1.02 mmol). The resulting reaction mixture was stirred at rt overnight. Then added water, the aqueous phase was extracted with dichloromethane, the combined organic phases were dried over anhydrous sodium sulfate, filtered, and concentrated in vacuo, purified by prep-HPLC to give SU20666-0134 (20 mg, yield: 37.7%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.40%, Rt=1.834 min; MS Calcd.: 446.1; MS Found: 447.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=8.739 min.

¹H NMR (400 MHz, DMSO-d₆) δ 2.02 (3H, s), 3.54 (2H, t, J=6.0 Hz), 4.21 (2H, t, J=6.4 Hz), 4.51 (2H, s), 6.92 (1H, dd, J=8.8 Hz, 3.2 Hz), 7.17-7.26 (3H, m), 7.35 (1H, d, J=8.0 Hz), 7.46 (1H, d, J=8.8 Hz), 7.75 (2H, d, J=9.6 Hz), 8.03 (1H, s), 8.27 (1H, t, J=5.6 Hz), 9.91 (1H, s).

The names SU20666-0135, SP 135, and 135 all refer to the same compound as shown below. The names SU20666-0143, SP 143, and 143 all refer to the same compound as shown below.

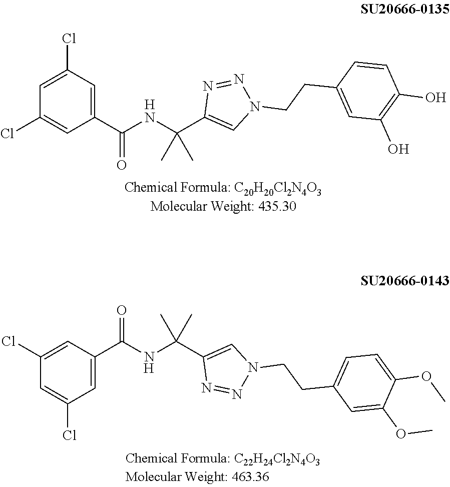

Route for SU20666-0135 and SU20666-0143

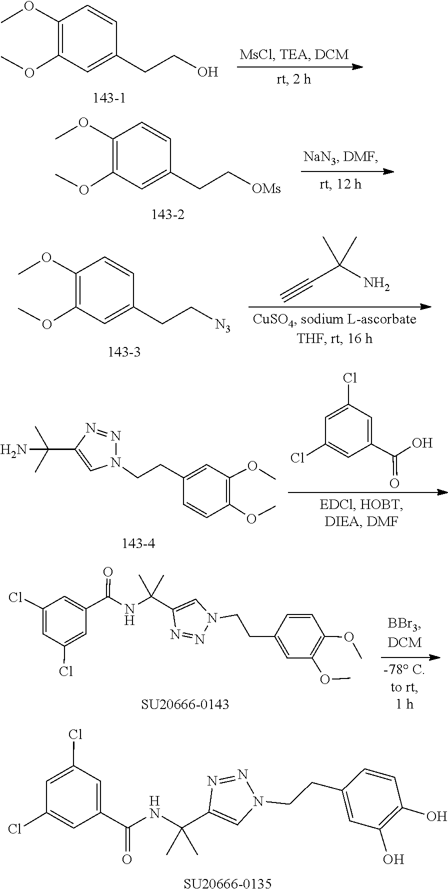

The synthesis of 3,4-dimethoxyphenethyl methanesulfonate (0143-2).

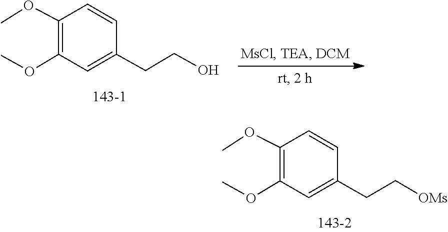 [SMILES: COc1ccc(CCO)cc1OC ; COc1ccc(CCOS(C)(=O)=O)cc1OC]

To a stirred solution of compound 0143-1 (1 g, 5.5 mmol) in DCM (10 ml) was added MsCl (0.7 ml, 8.2 mmol) and TEA (3.0 ml, 16.5 mmol) under ice-water. The resulting reaction mixture was stirred at 0° C. for 1 h. Then added water, the aqueous phase was extracted with DCM, the combined organic phases were dried over anhydrous sodium sulfate, filtered, and concentrated in vacuo to give the desired product 0143-2 (1.0 g, yield: 70%) as yellow oil.

The synthesis of 4-(2-azidoethyl)-1,2-dimethoxybenzene (0143-3).

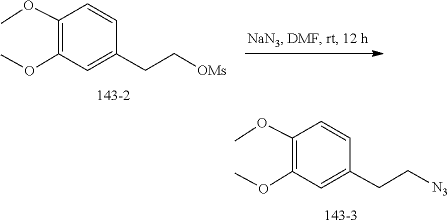 [SMILES: COc1ccc(CCOS(C)(=O)=O)cc1OC ; COc1ccc(CCN=[N+]=[N-])cc1OC]

To a solution of compound 0143-2 (1.0 g, 3.85 mmol) in DMF (10 mL) was added NaN₃ (0.50 g, 7.70 mmol). The mixture was stirred at 80° C. for 12 h. Then added water, the aqueous phase was extracted with EA, the combined organic phases were dried over anhydrous sodium sulfate, filtered, and concentrated in vacuo , purified by silical to give the desired product 0143-3 (0.5 g, yield: 62.5%) as a yellow solid.

The synthesis of 2-(1-(3,4-dimethoxyphenethyl)-1H-1,2,3-triazol-4-yl)propan-2-amine (0143-4).

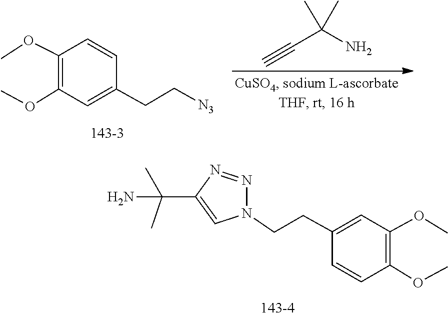

A solution of 143-3 (500 mg, 2.4 mmol) in THF/H₂O (10 ml/2 ml), was added 2-methylbut-3-yn-2-amine (200 mg, 2.4 mmol), CuSO₄ (300 mg, 1.2 mmol) and sodium L-ascorbate (240 mg, 1.2 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 0143-4 (300 mg, yield: 42.9%) as a green solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 71.27%, Rt=0.463 min; MS Calcd.: 290.2; MS Found: 291.4 [M+H]⁺.

The synthesis of 3,5-dichloro-N-(2-(1-(3,4-dimethoxyphenethyl)-1H-1,2,3-triazol-4-yl)propan-2-yl)benzamide (SU20666-0143).

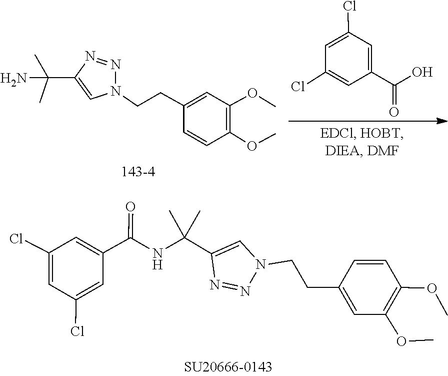

To a stirred solution of compound 0143-4 (200 mg, 0.69 mmol) in DCM (8 ml) was added 3,5-dichlorobenzoic acid (110 mg, 0.57 mmol), EDCI (165 mg, 0.86 mmol), HOBt (116 mg, 0.86 mmol) and DIEA (0.3 ml, 1.71mmol). The resulting reaction mixture was stirred at rt overnight. Then added water, the aqueous phase was extracted with dichloromethane, the combined organic phases were dried over anhydrous sodium sulfate, filtered, and concentrated in vacuo , purified by prep-HPLC to give SU20666-0143 (80 mg, yield: 30%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.10%, Rt=2.098 min; MS Calcd.: 462.1; MS Found: 463.3 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.42%, Rt=10.100 min.

¹H NMR (400 MHz, DMSO-d₆) δ 1.68 (6H, s), 3.04 (2H, t, J=7.2 Hz), 3.69 (6H, s), 4.49 (2H, t, J=7.2 Hz), 6.66-6.81 (3H, m), 7.79-7.88 (4H, m), 8.60 (1H, s).

The synthesis of 3,5-dichloro-N-(2-(1-(3,4-dihydroxyphenethyl)-1H-1,2,3-triazol-4-yl)propan-2-yl)benzamide (SU20666-0135).

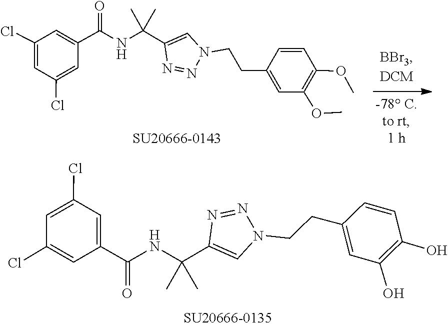 [SMILES: COc1ccc(CCn2cc(C(C)(C)NC(=O)c3cc(Cl)cc(Cl)c3)nn2)cc1OC ; CC(C)(NC(=O)c1cc(Cl)cc(Cl)c1)c1cn(CCc2ccc(O)c(O)c2)nn1]

To a stirred solution of compound SU20666-0143 (300 mg, 0.65 mmol) in DCM (10 ml) was added BBr₃ (0.68 M, 2 mL, 1.30 mmol) at −78° C. The resulting reaction mixture was warmed to rt and stirred for 1 h at rt. Then added water, the aqueous phase was extracted with dichloromethane, the combined organic phases were dried over anhydrous sodium sulfate, filtered, and concentrated in vacuo , purified by prep-HPLC to give SU20666-0135 (120 mg, yield: 43%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05 TFA] in 1.6 min, then under this condition for 1.4 min, finally changed to 95% [water+0.05% TFA] and 5% [CH₃CN+0.05% TFA] in 0.05 min and under this condition for 0.7 min), Purity: 98.69%, Rt=1.700 min; MS Calcd.: 434.1; MS Found: 435.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=8.515 min.

¹H NMR (400 MHz, DMSO-d₆) δ 1.68 (6H, s), 2.93 (2H, t, J=7.2 Hz), 4.14 (2H, t, J=7.2 Hz), 6.40 (1H, dd, J=8.0, 2.0 Hz), 6.56-6.61 (2H, m), 7.79-7.89 (4H, m), 8.61 (1H, s), 8.75 (2H, d, J=9.2 Hz).

The names SU20666-0136, SP 136, and 136 all refer to the same compound having the formula:

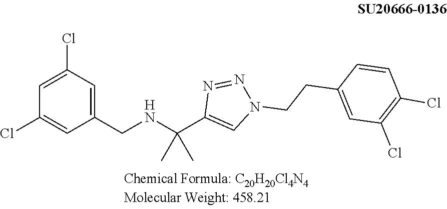

Route for SU20666-0136

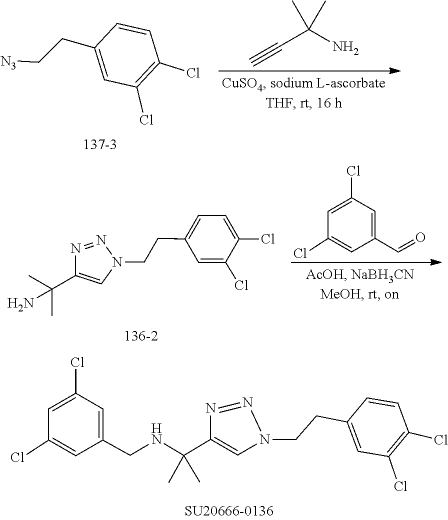

The synthesis of 2-(1-(3,4-dichlorophenethyl)-1H-1,2,3-triazol-4-yl)propan-2-amine (136-2).

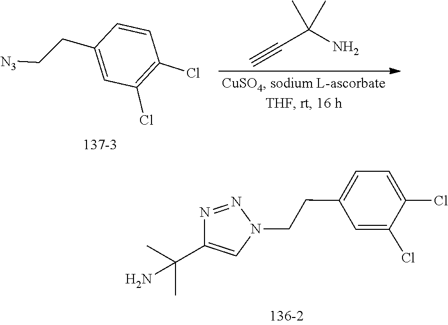

A solution of 137-3 (250 mg, 3.0 mmol) in THF/H₂O (10 ml/2 ml), was added 2-methylbut-3-yn-2-amine (650 mg, 3.0 mmol), CuSO₄ (374 mg, 1.5 mmol) and sodium L-ascorbate (297 mg, 1.5 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 0136-2 (400 mg, yield: 45%) as a green solid.

The synthesis of N-(3,5-dichlorobenzyl)-2-(1-(3,4-dichlorophenethyl)-1H-1,2,3-triazol-4-yl)propan-2-amine (SU20666-0136).

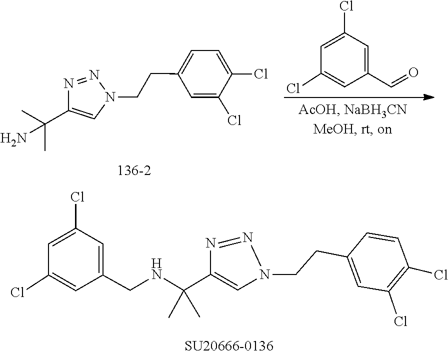

A solution of 136-2 (200 mg, 1.0 mmol) in MeOH/HOAc (10 ml/0.5 ml), was added 3,5-dichlorobenzaldehyde (175 mg, 1.0 mmol) and NaBH₃CN (190 mg, 3.0 mmol). The mixture was stirred at rt for 16 h. Then added water, the aqueous phase was extracted with EA, the combined organic phases were dried over anhydrous sodium sulfate, filtered, and concentrated in vacuo to give the desired product SU20666-0136 (35 mg, yield: 7.7%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.45%, Rt=2.515 min; MS Calcd.: 456.0; MS Found: 457.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=12.411 min.

¹H NMR (400 MHz, DMSO-d₆) δ 1.35 (6H, s), 3.13 (2H, t, J=7.2 Hz), 3.33 (2H, s), 4.56 (2H, t, J=6.8 Hz), 7.11 (1H, dd, J=8.4, 2.0 Hz), 7.28 (2H, d, J=3.2 Hz), 7.35-7.44 (3H, m), 7.80 (1H, s).

The names SU20666-0137, SP 137, and 137 all refer to the same compound having the formula:

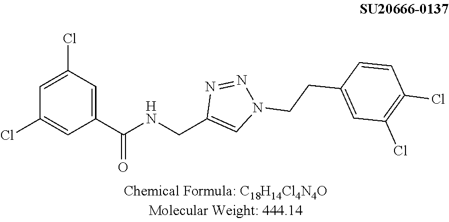

Route for SU20666-0137

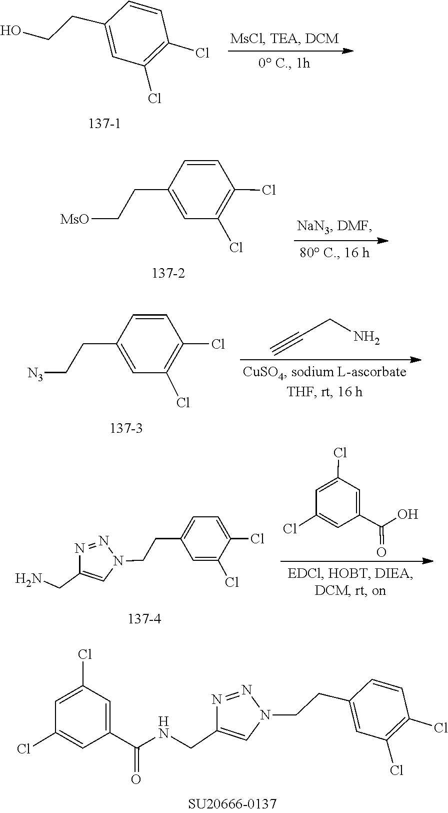

The synthesis of 3,4-dichlorophenethyl methanesulfonate (137-2).

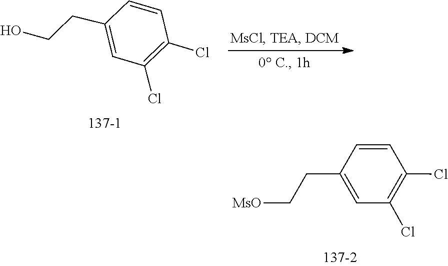 [SMILES: OCCc1ccc(Cl)c(Cl)c1 ; CS(=O)(=O)OCCc1ccc(Cl)c(Cl)c1]

To a stirred solution of compound 137-1 (2 g, 10.5 mmol) in DCM (20 ml) was added MsCl (1.8 g, 15.7 mmol) and TEA (5.6 ml, 31.5 mmol) under ice-water. The resulting reaction mixture was stirred at 0° C. for 1 h. Then added water, the aqueous phase was extracted with DCM, the combined organic phases were dried over anhydrous sodium sulfate, filtered, and concentrated in vacuo to give the desired product 137-2 (1.5 g, yield: 53.6%) as yellow oil.

The synthesis of 4-(2-azidoethyl)-1,2-dichlorobenzene (137-3).

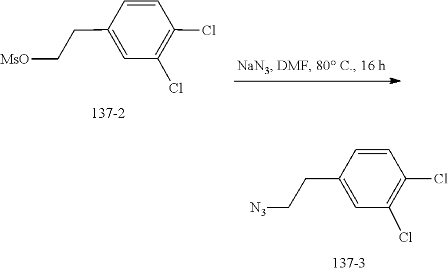 [SMILES: CS(=O)(=O)OCCc1ccc(Cl)c(Cl)c1 ; [N-]=[N+]=NCCc1ccc(Cl)c(Cl)c1]

To a solution of compound 137-2 (1.5 g, 5.6 mmol) in DMF (15 mL) was added NaN₃ (0.73 g, 11.2 mmol). The mixture was stirred at 80° C. for 16 h. Then added water, the aqueous phase was extracted with EA, the combined organic phases were dried over anhydrous sodium sulfate, filtered, and concentrated in vacuo , purified by silical to give the desired product 137-3 (0.9 g, yield: 75%) as a yellow solid.

The synthesis of (1-(3,4-dichlorophenethyl)-1H-1,2,3-triazol-4-yl)methanamine (137-4).

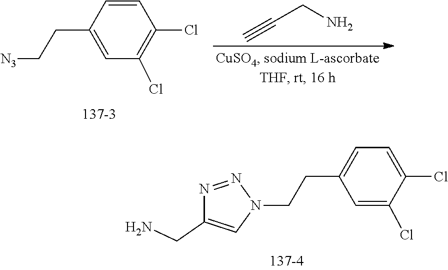

A solution of 137-3 (500 mg, 2.3 mmol) in THF/H₂O (10 ml/2 ml), was added prop-2-yn-1-amine (128 mg, 2.3 mmol), CuSO₄ (288 mg, 1.16 mmol) and sodium L-ascorbate (230 mg, 1.16 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 137-4 (400 mg, yield: 63.7%) as a green solid.

The synthesis of 3,5-dichloro-N-((1-(3,4-dichlorophenethyl)-1H-1,2,3-triazol-4-yl)methyl)benzamide (SU20666-0137).

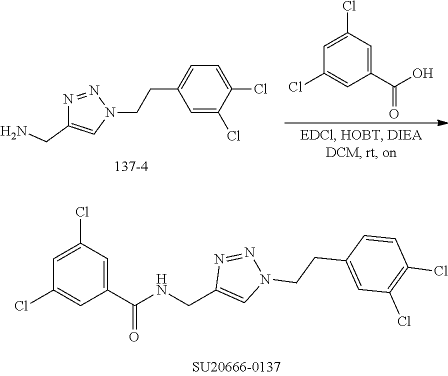 [SMILES: NCc1cn(CCc2ccc(Cl)c(Cl)c2)nn1 ; O=C(O)c1cc(Cl)cc(Cl)c1 ; O=C(NCc1cn(CCc2ccc(Cl)c(Cl)c2)nn1)c1cc(Cl)cc(Cl)c1]

To a stirred solution of compound 137-4 (200 mg, 0.74 mmol) in DCM (8 ml) was added 3,5-dichlorobenzoic acid (118 mg, 0.62 mmol), EDCI (180 mg, 0.93 mmol), HOBt (126 mg, 0.93 mmol) and DIEA (0.33 ml, 1.86 mmol). The resulting reaction mixture was stirred at rt overnight. Then added water, the aqueous phase was extracted with dichloromethane, the combined organic phases were dried over anhydrous sodium sulfate, filtered, and concentrated in vacuo , purified by prep-HPLC to give SU20666-0137 (10 mg, yield: 3.7%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.80%, Rt=2.230 min; MS Calcd.: 442.0; MS Found: 443.0[M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=10.964 min.

¹H NMR (400 MHz, DMSO-d₆) δ 3.12 (2H, t, J=7.2 Hz), 4.44 (2H, d, J=5.2 Hz), 4.57 (2H, t, J=7.2 Hz), 7.12 (1H, d, J=8.0 Hz), 7.46 (2H, d, J=8.4 Hz), 7.81-7.91 (4H, m), 9.24 (1H, s).

The names SU20666-0138, SP 138, and 138 all refer to the same compound having the formula:

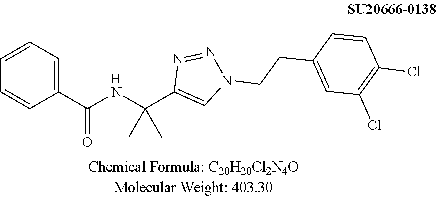

Route for SU20666-0138

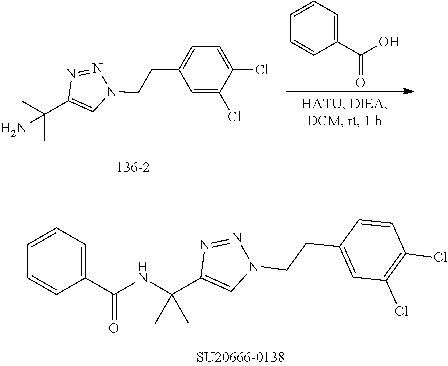

The synthesis of N-(2-(1-(3,4-dichlorophenethyl)-1H-1,2,3-triazol-4-yl)propan-2-yl)benzamide (SU20666-0138).

To a stirred solution of compound 0136-2 (300 mg, 1.0 mmol) in DCM (8 ml) was added benzoic acid (146 mg, 1.2 mmol), HATU (570 mg, 1.5 mmol) and DIEA (387 mg, 3.0 mmol). The resulting reaction mixture was stirred at rt for 1 h. Then added water, the aqueous phase was extracted with dichloromethane, the combined organic phases were dried over anhydrous sodium sulfate, filtered, and concentrated in vacuo , purified by prep-HPLC to give SU20666-0138 (20 mg, yield: 5%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.38%, Rt=2.089 min; MS Calcd.: 402.1; MS Found: 403.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 95.42%, Rt=9.561 min.

¹H NMR (400 MHz, DMSO-d₆) δ 1.66 (6H, s), 3.11 (2H, t, J=7.2 Hz), 4.53 (2H, t, J=7.2 Hz), 7.09 (1H, dd, J=8.4, 2.0 Hz), 7.40-7.49 (5H, m), 7.78 (2H, d, J=8.4 Hz), 7.82 (1H, s), 8.27 (1H, s).

The names SU20666-0139, SP 139, and 139 all refer to the same compound having the formula:

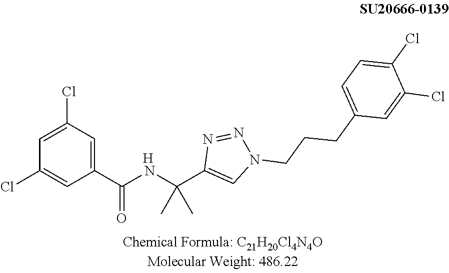

Route for SU20666-0139

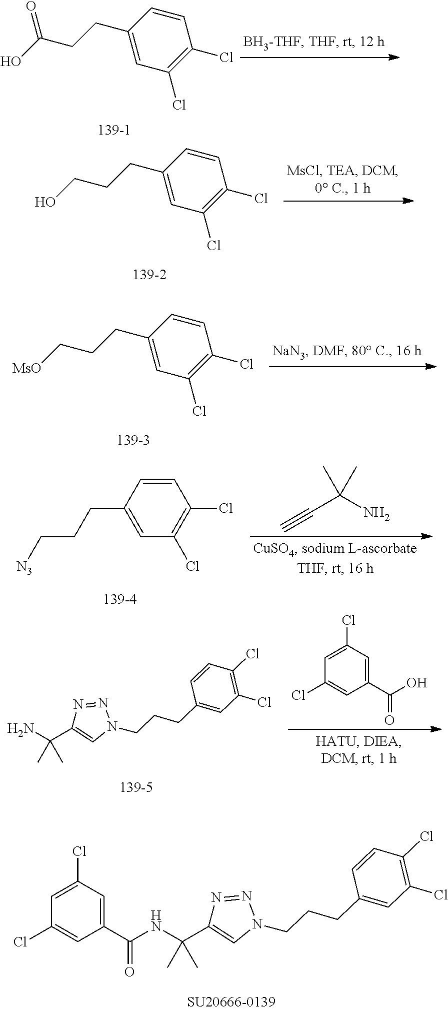

The synthesis of 3-(3,4-dichlorophenyl)propan-1-ol (139-2).

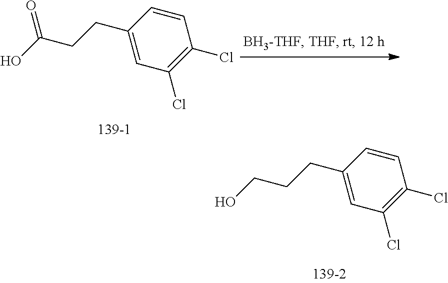 [SMILES: O=C(O)CCc1ccc(Cl)c(Cl)c1 ; OCCCc1ccc(Cl)c(Cl)c1]

To a stirred solution of 0139-1 (2.0 g, 9.2 mmol) in THF (20 ml) was added borane-tetrahydrofuran (1.0 N, 55 mL, 55 mmol). The resulting reaction mixture was stirred for 12 h. Then added HCl (1.0 N, 3 mL) and stirred for 1 h at rt, the aqueous phase was neutralized and then extracted with dichloromethane, the combined organic phases were dried over anhydrous sodium sulfate, filtered, and concentrated in vacuo, to give the desired product 139-2 (15 mg, yield: 7.9%) as a white solid.

The synthesis of 3-(3,4-dichlorophenyl)propyl methanesulfonate (139-3).

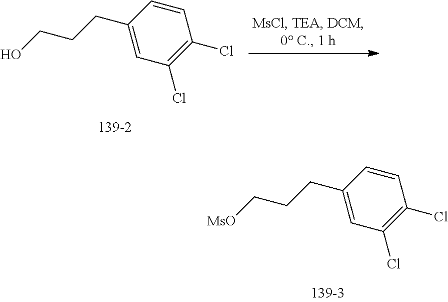 [SMILES: OCCCc1ccc(Cl)c(Cl)c1 ; CS(=O)(=O)OCCCc1ccc(Cl)c(Cl)c1]

To a stirred solution of compound 139-2 (2 g, 10.0 mmol) in DCM (20 ml) was added MsCl (1.7 g, 15.0 mmol) and TEA (3.0 g, 30.0 mmol) under ice-water. The resulting reaction mixture was stirred at 0° C. for 1 h. Then added water, the aqueous phase was extracted with DCM, the combined organic phases were dried over anhydrous sodium sulfate, filtered, and concentrated in vacuo to give the desired product 139-3 (2.5 g, yield: 91%) as yellow oil.

The synthesis of 4-(3-azidopropyl)-1,2-dichlorobenzene (139-4).

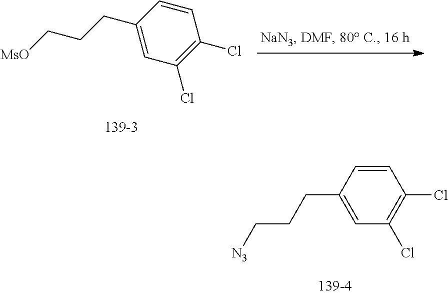 [SMILES: CS(=O)(=O)OCCCc1ccc(Cl)c(Cl)c1 ; [N-]=[N+]=NCCCc1ccc(Cl)c(Cl)c1]

To a solution of compound 0139-3 (3.0 g, 10.6 mmol) in DMF (10 mL) was added NaN₃ (1.4 g, 21.3 mmol). The mixture was stirred at 80° C. for 12 h. Then added water, the aqueous phase was extracted with EA, the combined organic phases were dried over anhydrous sodium sulfate, filtered, and concentrated in vacuo , purified by silical to give the desired product 0139-4 (2.5 g, yield: 100%) as a yellow solid.

The synthesis of 2-(1-(3-(3,4-dichlorophenyl)propyl)-1H-1,2,3-triazol-4-yl)propan-2-amine (0139-5).

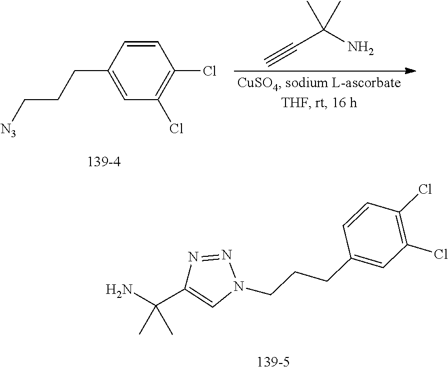

A solution of 139-4 (2.5 g, 10.9 mmol) in THF/H₂O (25 ml/5 ml), was added 2-methylbut-3-yn-2-amine (0.90 g, 10.9 mmol), CuSO₄ (1.4 g, 5.4 mmol) and sodium L-ascorbate (1.1 g, 5.4 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 0139-5 (2.0 g, yield: 59%) as a green solid.

The synthesis of 3,5-dichloro-N-(2-(1-(3-(3,4-dichlorophenyl)propyl)-1H-1,2,3-triazol-4-yl)propan-2-yl)benzamide (SU20666-0139).

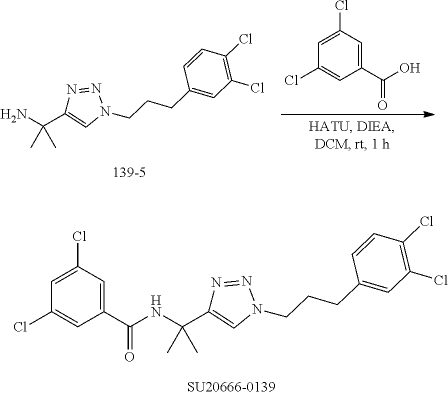 [SMILES: CC(C)(N)c1cn(CCCc2ccc(Cl)c(Cl)c2)nn1 ; O=C(O)c1cc(Cl)cc(Cl)c1 ; CC(C)(NC(=O)c1cc(Cl)cc(Cl)c1)c1cn(CCCc2ccc(Cl)c(Cl)c2)nn1]

To a stirred solution of compound 0139-5 (197 mg, 0.63 mmol) in DCM (5 ml) was added 3,5-dichlorobenzoic acid (100 mg, 0.52 mmol), HATU (298 mg, 0.79 mmol) and DIEA (202 mg, 1.57 mmol). The resulting reaction mixture was stirred at rt for 1 h. Then added water, the aqueous phase was extracted with dichloromethane, the combined organic phases were dried over anhydrous sodium sulfate, filtered, and concentrated in vacuo , purified by prep-HPLC to give SU20666-0139 (77 mg, yield: 30%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.78%, Rt=2.451 min; MS Calcd.: 484.0; MS Found: 485.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=12.015 min.

¹H NMR (400 MHz, DMSO-d₆) δ 1.69 (6H, s), 2.10-2.14 (2H, m), 2.52-2.56 (2H, m), 4.30 (2H, t, J=7.2 Hz), 7.21 (1H, dd, J=8.4, 2.0 Hz), 7.48-7.53 (2H, m), 7.79 (1H, s), 7.85 (2H, d, J=2.0 Hz), 7.96 (1H, s), 8.61 (1H, s).

SU-20666-0141

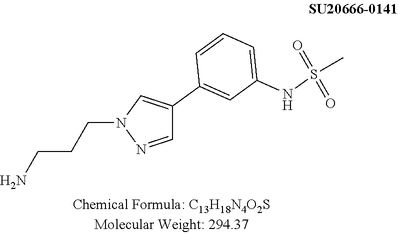 [SMILES: CS(=O)(=O)Nc1cccc(-c2cnn(CCCN)c2)c1]

Route for SU-20666-0141

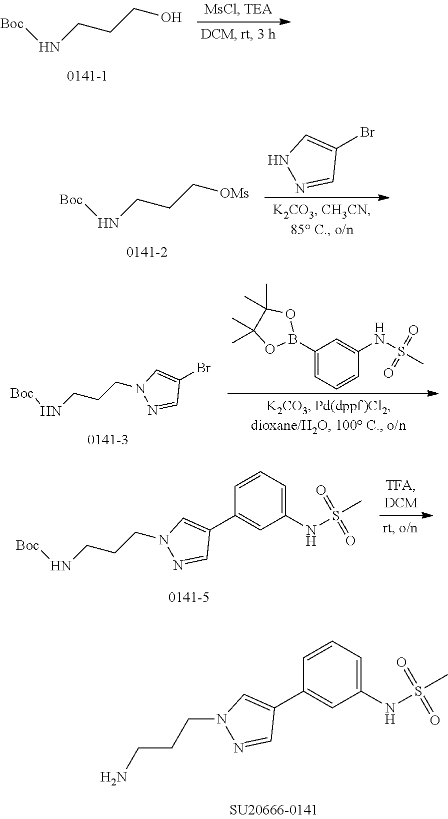

The synthesis of 3-(tert-butoxycarbonylamino)propyl methanesulfonate (0141-2).

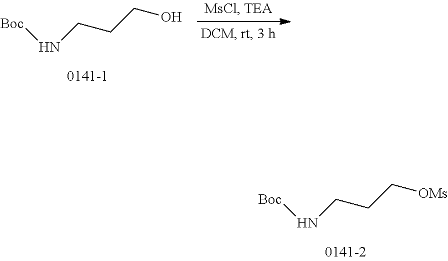 [SMILES: CC(C)(C)OC(=O)NCCCO ; CC(C)(C)OC(=O)NCCCOS(C)(=O)=O]

To a solution of tert-butyl 3-hydroxypropylcarbamate (5 g, 28.6 mmol) in DCM (20 mL) was added MsCl (4.92 mg, 42.9 mmol) and TEA (8.7 g, 85.8 mmol). The mixture was stirred at rt for 3 h. Then concentrated in vacuo to give crude compound. The crude product was purified by pre-HPLC to afford 3-(tert-butoxycarbonylamino)propyl methanesulfonate (7.6 g, 100%) as a yellow solid.

The synthesis of tert-butyl 3-(4-bromo-1H-pyrazol-1-yl)propylcarbamate (0141-3)

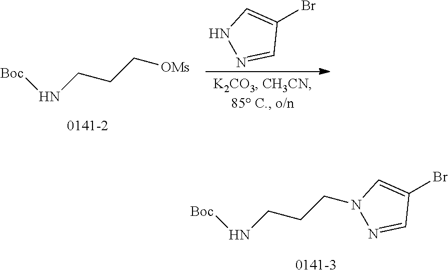 [SMILES: CC(C)(C)OC(=O)NCCCOS(C)(=O)=O ; Brc1cn[nH]c1 ; CC(C)(C)OC(=O)NCCCn1cc(Br)cn1]

To a solution of 3-(tert-butoxycarbonylamino)propyl methanesulfonate (7.6 g, 30 mmol) in CH₃CN (30 mL) was added 4-bromo-1H-pyrazole (2.94 g, 20 mmol) and K₂CO₃ (5.52 g, 40 mmol). The mixture was stirred at 85° C. for o/n. Then concentrated in vacuo to give tert-butyl 3-(4-bromo-1H-pyrazol-1-yl)propylcarbamate (2 g, 20%) as a yellow solid.

The synthesis of tert-butyl 3-(4-(3-(methylsulfonamido)phenyl)-1H-pyrazol-1-yl)propylcarbamate (0141-4).

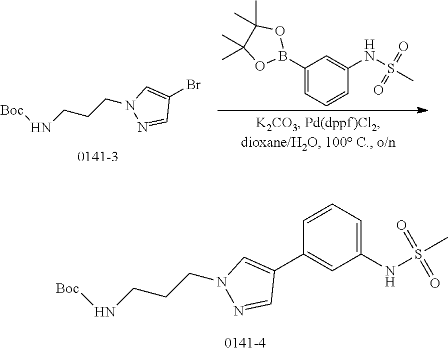

To a solution of tert-butyl 3-(4-bromo-1H-pyrazol-1-yl)propylcarbamate (1.8 g, 5.94 mmol) in dioxane/H₂O (20/2 mL) was added N-(3-(4,4,5,5-tetramethyl-1,3,2-dioxaborolan-2-yl)phenyl)methanesulfonamide (2.2 g, 7.2 mmol), Pd(dppf)Cl₂ (180 mg) and K₂CO₃ (2.46 g, 17.8 mmol), N₂ protected. The mixture was stirred at 100° C. for o/n. Then concentrated in vacuo to give crude compound. The crude product was purified by pre-HPLC to afford tert-butyl 3-(4-(3-(methylsulfonamido)phenyl)-1H-pyrazol-1-yl)propylcarbamate (600 mg, 26%) as a yellow solid.

The synthesis of N-(3-(1-(3-aminopropyl)-1H-pyrazol-4-yl) phenyl)methanesulfonamide (SU20666-0130).

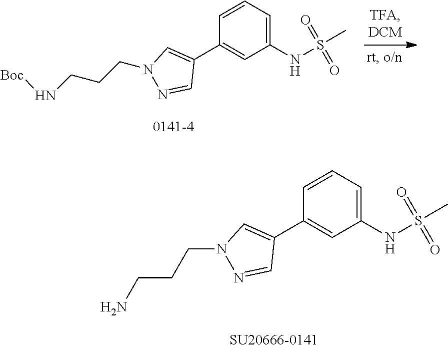 [SMILES: CC(C)(C)OC(=O)NCCCn1cc(-c2cccc(NS(C)(=O)=O)c2)cn1 ; CS(=O)(=O)Nc1cccc(-c2cnn(CCCN)c2)c1]

To a solution of tert-butyl 3-(4-(3-(methylsulfonamido) phenyl)-1H-pyrazol-1-yl)propylcarbamate (500 mg, 1.3 mmol) DCM (10 mL) was added TFA (5 mL). The mixture was stirred at rt for o/n. Then concentrated in vacuo to give compound. N-(3-(1-(3-aminopropyl)-1H-pyrazol-4-yl)phenyl) methanesulfonamide (260 mg, 68%) as a white solid. LC-MS (Agilent LCMS 1200-6120, Column: Waters X-Bridge C18 (50 mm×4.6 mm×3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.), Purity is 98.47%, Rt=1.226 min; MS Calcd.: 294.1; MS Found: 295.1 [M+H]⁺.

HPLC (Agilent HPLC 1200, Column: Waters X-Bridge C18 (150 mm×4.6 mm×3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity is 100%, Rt=5.999 min.

¹H NMR (400 MHz, DMSO) 6 8.11 (s, 1H), 7.76 (s, 1H), 7.26 (dd, J=17.4, 10.1 Hz, 3H), 6.99 (d, J=7.1 Hz, 1H), 6.42 (d, J=300.2 Hz, 1H), 4.28 (s, 2H), 4.11 (t, J=6.7 Hz, 2H), 2.95 (s, 3H), 2.90 (d, J=6.3 Hz, 2H), 1.95-1.75 (m, 2H).

The names SU20666-0146, SP 146, and 146 all refer to the same compound having the formula:

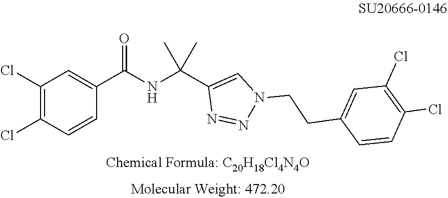 [SMILES: CC(C)(NC(=O)c1ccc(Cl)c(Cl)c1)c1cn(CCc2ccc(Cl)c(Cl)c2)nn1]

Route for SU20666-0146

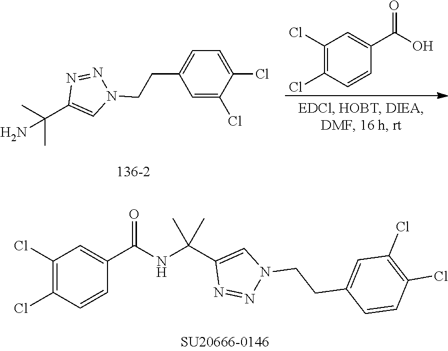 [SMILES: CC(C)(N)c1cn(CCc2ccc(Cl)c(Cl)c2)nn1 ; O=C(O)c1ccc(Cl)c(Cl)c1 ; CC(C)(NC(=O)c1ccc(Cl)c(Cl)c1)c1cn(CCc2ccc(Cl)c(Cl)c2)nn1]

The synthesis of 3,4-dichloro-N-(2-(1-(3,4-dichlorophenethyl)-1H-1,2,3-triazol-4-yl)propan-2-yl)benzamide (SU20666-0146).

To a stirred solution of compound 0136-2 (100 mg, 0.33 mmol) in DMF (5 ml) was added 3,4-dichlorobenzoic acid (54 mg, 0.28 mmol), EDCI (80 mg, 0.42 mmol), HOBT (57 mg, 0.42 mmol) and DIEA (108 mg, 0.84 mmol). The resulting reaction mixture was stirred at rt for 16 h. Then added water, the aqueous phase was extracted with dichloromethane, the combined organic phases were dried over anhydrous sodium sulfate, filtered, and concentrated in vacuo , purified by prep-HPLC to give SU20666-0146 (30 mg, yield: 23%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.90%, Rt=2.321 min; MS Calcd.: 470.0; MS Found: 471.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 96.62%, Rt=11.382 min.

¹H NMR (400 MHz, DMSO-d₆) δ 1.67 (6H, s), 3.13 (2H, t, J=7.2 Hz), 4.54 (2H, t, J=7.2 Hz), 7.11 (1H, d, J=8.4 Hz), 7.41 (1H, s), 7.46 (1H, d, J=8.4 Hz), 7.72-7.80 (2H, m), 7.86 (1H, s), 8.09 (1H, s), 8.54 (1H, s).

The names SU20666-0147, SP 147, and 147 all refer to the same compound having the formula:

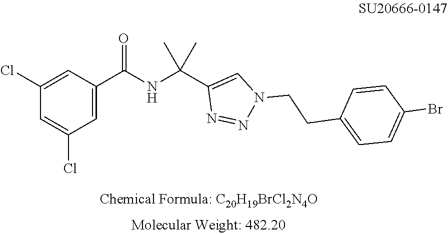 [SMILES: CC(C)(NC(=O)c1cc(Cl)cc(Cl)c1)c1cn(CCc2ccc(Br)cc2)nn1]

Route for SU20666-0147

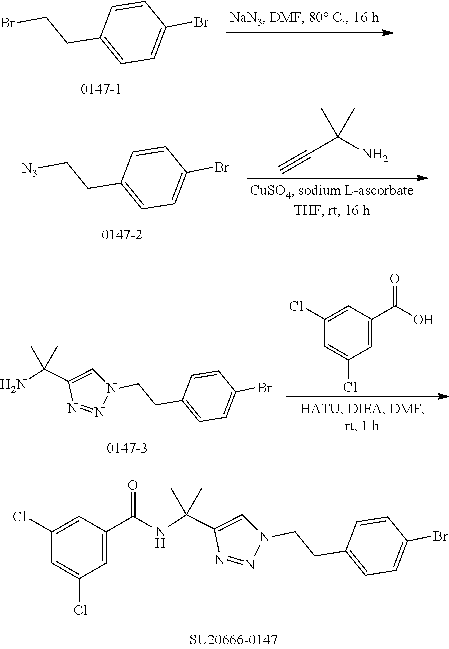

The synthesis of 1-(2-azidoethyl)-4-bromobenzene (0147-2).

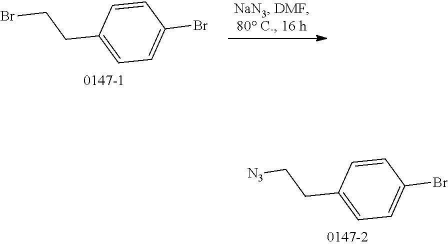 [SMILES: BrCCc1ccc(Br)cc1 ; [N-]=[N+]=NCCc1ccc(Br)cc1]

To a solution of compound 0147-1 (5 g, 18.9 mmol) in DMF (20 mL) was added NaN₃ (2.46 g, 37.9 mmol). The mixture was stirred at 80° C. for 16 h. Then added water, the aqueous phase was extracted with EA, the combined organic phases were dried over anhydrous sodium sulfate, filtered, and concentrated in vacuo to give the desired product 0147-2 (4 g, yield: 94%) as a yellow solid.

The synthesis of 2-(1-(4-bromophenethyl)-1H-1,2,3-triazol-4-yl)propan-2-amine (0147-3).

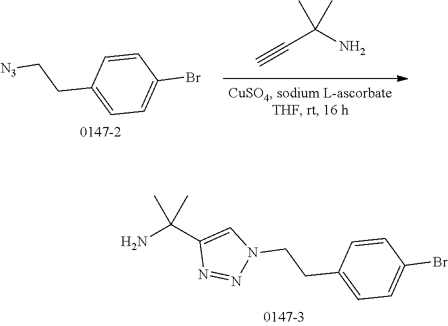

A solution of 0147-2 (4 g, 17.8 mmol) in THF/H₂O (20 ml/4 ml), was added 2-methylbut-3-yn-2-amine (1.48 g, 17.8 mmol), CuSO₄ (2.2 g, 8.9 mmol) and sodium L-ascorbate (1.76 g, 8.9 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 0147-3 (2 g, yield: 36.5%) as a green solid.

The synthesis of N-(2-(1-(4-bromophenethyl)-1H-1,2,3-triazol-4-yl)propan-2-yl)-3,5-dichlorobenzamide (SU20666-0147).

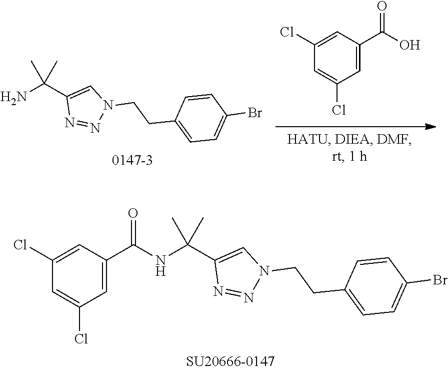 [SMILES: CC(C)(N)c1cn(CCc2ccc(Br)cc2)nn1 ; O=C(O)c1cc(Cl)cc(Cl)c1 ; CC(C)(NC(=O)c1cc(Cl)cc(Cl)c1)c1cn(CCc2ccc(Br)cc2)nn1]

To a stirred solution of compound 0147-3 (200 mg, 0.65 mmol) in DMF (5 ml) was added 3,5-dichlorobenzoic acid (100 mg, 0.54 mmol), HATU (300 mg, 0.81 mmol) and DIEA (0.3 ml, 1.62 mmol). The resulting reaction mixture was stirred at rt overnight. Then added water, the aqueous phase was extracted with dichloromethane, the combined organic phases were dried over anhydrous sodium sulfate, filtered, and concentrated in vacuo, purified by prep-HPLC to give SU20666-0147 (70 mg, yield: 28%) as a white solid.

LC-MS (Agilent LCMS 1200-6120, Column: Waters X-Bridge C18 (50 mm*4.6 mm*3.5μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.23%, Rt=2.312 min; MS Calcd.: 480.0; MS Found: 481.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 98.8%, Rt=11.297 min.

¹H NMR (400 MHz, DMSO-d₆) δ 1.67 (6H, s), 3.10 (2H, t, J=6.8 Hz), 4.52 (2H, t, J=7.2 Hz), 7.08 (2H, d, J=8.4 Hz), 7.40 (2H, d, J=8.4 Hz), 7.79-7.86 (4H, m), 8.59 (1H, s).

SU20666-0148

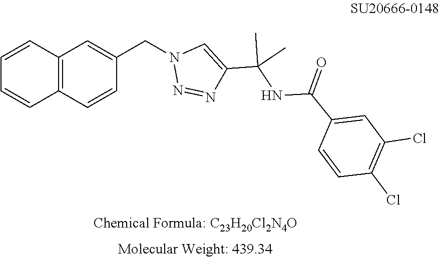 [SMILES: CC(C)(NC(=O)c1ccc(Cl)c(Cl)c1)c1cn(Cc2ccc3ccccc3c2)nn1]

Route for SU20666-0148

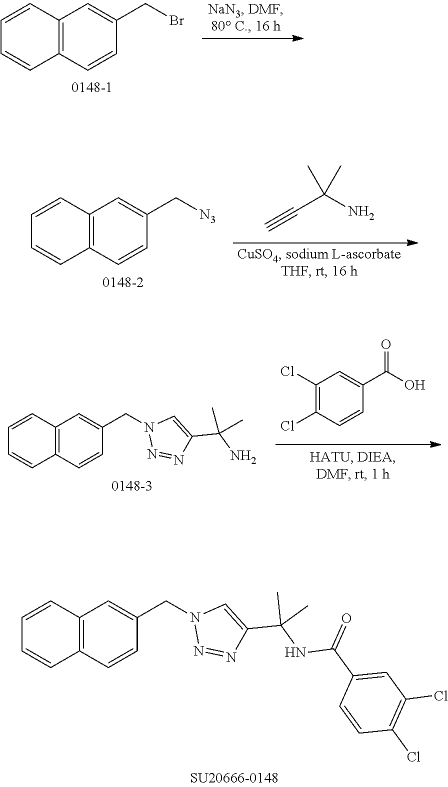

The synthesis of 2-(azidomethyl)naphthalene (0148-2).

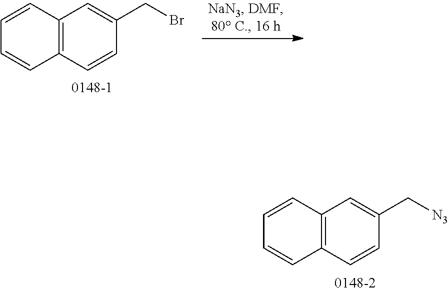 [SMILES: BrCc1ccc2ccccc2c1 ; [N-]=[N+]=NCc1ccc2ccccc2c1]

To a solution of compound 0148-1 (5 g, 22.6 mmol) in DMF (20 mL) was added NaN₃ (2.9 g, 45.2 mmol). The mixture was stirred at 80° C. for 16 h. Then added water, the aqueous phase was extracted with EA, the combined organic phases were dried over anhydrous sodium sulfate, filtered, and concentrated in vacuo to give the desired product 0148-2 (3 g, yield: 72.5%) as a yellow solid.

The synthesis of 2-(1-(naphthalen-2-ylmethyl)-1H-1,2,3-triazol-4-yl)propan-2-amine (0148-3).

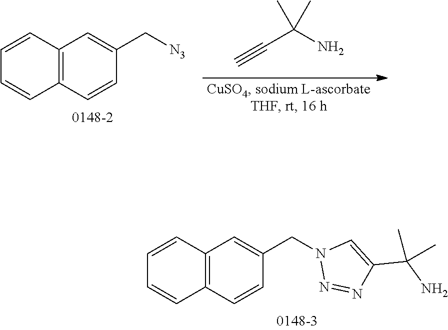

A solution of 0148-2 (3 g, 16.4 mmol) in THF/H₂O (20 ml/4 ml), was added 2-methylbut-3-yn-2-amine (1.36 g, 16.4 mmol), CuSO₄ (2.0 g, 8.2 mmol) and sodium L-ascorbate (1. 6 g, 8.2 mmol). The mixture was stirred at rt for 16 h. The solvent was removed, added water, the aqueous phase was extracted with DCM, the combined organic phases were dried over anhydrous sodium sulfate, filtered, and concentrated in vacuo to give the desired product 0148-3 (1.5 g, yield: 34%) as a green solid.

The synthesis of 3,4-dichloro-N-(2-(1-(naphthalen-2-ylmethyl)-1H-1,2,3-triazol-4-yl)propan-2-yl)benzamide (SU20666-0148).

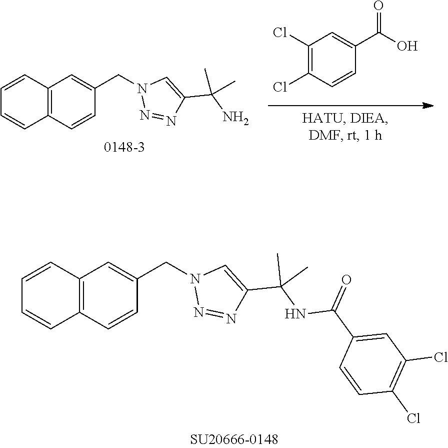 [SMILES: CC(C)(N)c1cn(Cc2ccc3ccccc3c2)nn1 ; O=C(O)c1ccc(Cl)c(Cl)c1 ; CC(C)(NC(=O)c1ccc(Cl)c(Cl)c1)c1cn(Cc2ccc3ccccc3c2)nn1]

To a stirred solution of compound 0148-3 (200 mg, 0.75 mmol) in DMF (5 ml) was added 3,4-dichlorobenzoic acid (119 mg, 0.63 mmol), HATU (357 mg, 0.94 mmol) and DIEA (0.4 ml, 1.88 mmol). The resulting reaction mixture was stirred at rt overnight. Then added water, the aqueous phase was extracted with dichloromethane, the combined organic phases were dried over anhydrous sodium sulfate, filtered, and concentrated in vacuo, purified by prep-HPLC to give SU20666-0148 (40 mg, yield: 14.5%) as a white solid.

LC-MS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.27%, Rt=2.263 min; MS Calcd.: 438.1; MS Found: 439.1 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=11.016 min.

¹H NMR (400 MHz, CDCl₃) δ 1.75 (6H, s), 5.62 (2H, s), 6.99 (1H, s), 7.33 (1H, d, J=1.6 Hz), 7.39-7.52 (5H, m), 7.72(1H, s), 7.78-7.81 (4H, m).

The names SU20666-150, SP 150, and 150 all refer to the same compound having the formula:

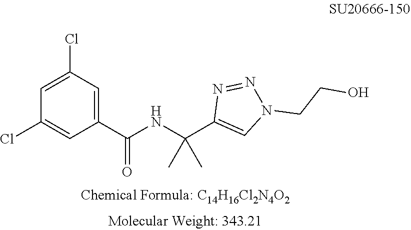 [SMILES: CC(C)(NC(=O)c1cc(Cl)cc(Cl)c1)c1cn(CCO)nn1]

Route for SU20666-150

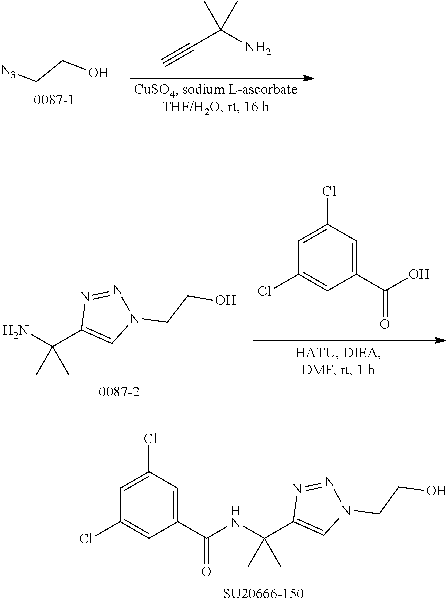

The synthesis of 2-(4-(2-aminopropan-2-yl)-1H-1,2,3-triazol-1-yl)ethanol (0087-2).

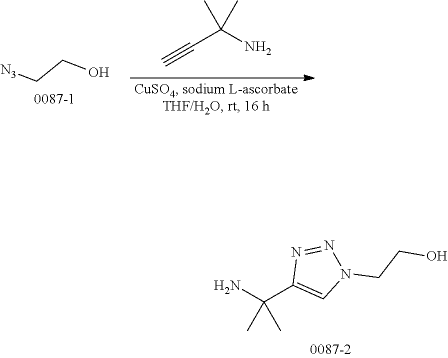

To a stirred solution of compound 0087-1 (1 g, 11.5 mmol) in THF/water (30 ml/6 ml) was added 2-methylbut-3-yn-2-amine (954 mg, 11.5 mmol), CuSO₄ (1.44 g, 5.75 mmol) and sodium L-ascorbate (1.14 g, 5.75 mmol). The resulting reaction mixture was stirred at rt for 16 h. Then removed solvent, added water, the aqueous phase was extracted with dichloromethane, the combined organic phases were dried over anhydrous sodium sulfate, filtered, and concentrated in vacuo to give the desired product 0087-2 (1.9 g, yield: 97.2%) as a green solid.

The synthesis of 3,5-dichloro-N-(2-(1-(2-hydroxyethyl)-1H-1,2,3-triazol-4-yl)propan-2-yl)benzamide (SU20666-150).

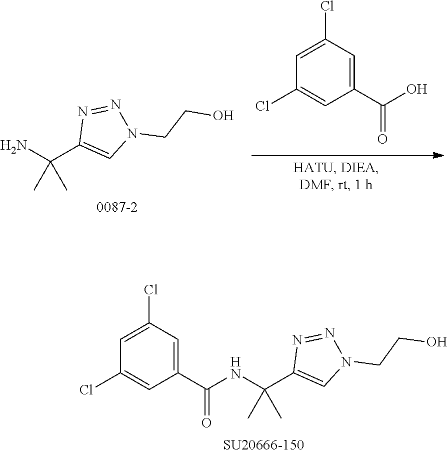

To a stirred solution of compound 0087-2 (1.9 g, 11.2 mmol) in DCM (20 ml) was added 3,5-dichlorobenzoic acid (1.4 g, 7.45 mmol), DIEA (2.9 g, 22.4 mmol) and HATU (4.0 g, 11.2 mmol). The resulting reaction mixture was stirred at rt for 1 h. Then added water, the aqueous phase was extracted with dichloromethane, the combined organic phases were dried over anhydrous sodium sulfate, filtered, and concentrated in vacuopurified by prep-HPLC to give the desired product SU20666-150 (1.5 g, yield: 60%) as white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.636 min; MS Calcd.: 342; MS Found: 343.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=7.767 min; MS Calcd.: 342; MS Found: 343.0 [M+H]⁺.

¹H NMR (400 MHz, DMSO-d₆) δ 1.70 (6H, s), 3.77 (2H, q, J=5.2 Hz), 4.34(2H, t, J=5.2 Hz), 5.01 (1H, t, J=5.2 Hz), 7.79 (1H, t, J=2.0 Hz), 7.84 (2H, d, J=2.0 Hz), 7.89 (1H, s), 8.58 (1H, s).

The names SU20666-151, SP 151, and 151 all refer to the same compound having the formula:

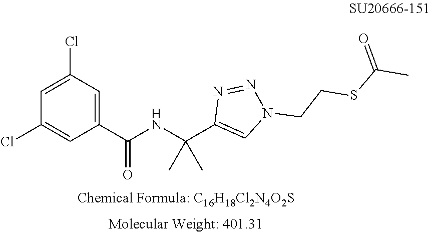 [SMILES: CC(=O)SCCn1cc(C(C)(C)NC(=O)c2cc(Cl)cc(Cl)c2)nn1]

Route for SU20666-151

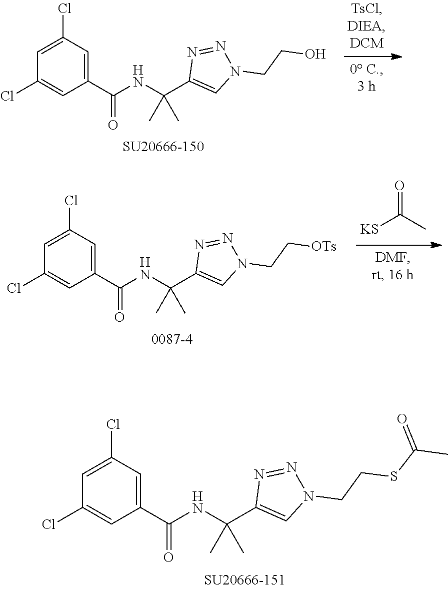

The synthesis of 2-(4-(2-(3,5-dichlorobenzamido)propan-2-yl)-1H-1,2,3-triazol-1-yl)ethyl 4-methylbenzenesulfonate (0087-4).

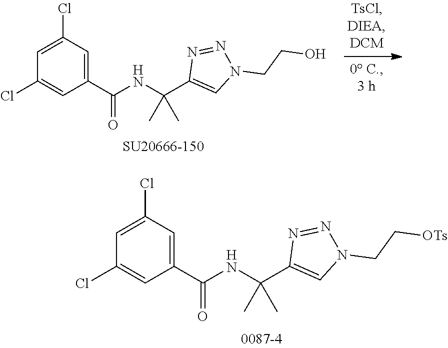

To a stirred solution of 150-01 (100 mg, 0.29 mmol) in DCM (15 ml) was added DIEA (113.1 mg, 0.875 mmol) and TsC₁ (82.94 mg, 0.435 mmol) at 0° C. The resulting reaction mixture was stirred for 3 h at 0° C. Then added water, the aqueous phase was extracted with dichloromethane, the combined organic phases were dried over anhydrous sodium sulfate, filtered, and concentrated in vacuo to give the desired product 0087-4 (100 mg, yield: 68%) as a white solid.

The synthesis of S-2-(4-(2-(3,5-dichlorobenzamido)propan-2-yl)-1H-1,2,3-triazol-1-yl)ethyl ethanethioate (SU20666-151).

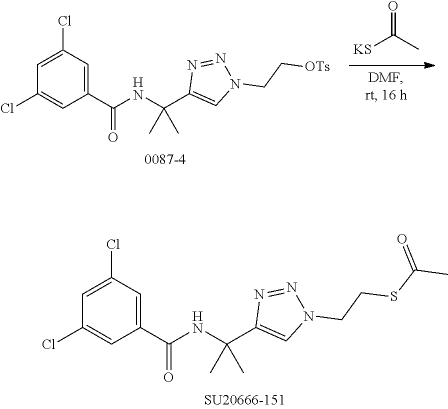

To a solution of compound 087-4 (50 mg, 0.1 mmol) in DMF (15 mL) was added potassium thioacetate (13.78 mg, 0.12 mmol). The mixture was stirred for 16 h at rt, after the consumption of starting material (by LCMS), the mixture was purified by prep-HPLC to give SU20666-151 (19.22 mg, yield: 47%) as a brown solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.985 min; MS Calcd.: 400; MS Found: 401.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 94.49%, Rt=9.513 min; MS Calcd.: 400; MS Found: 401.0 [M+H]⁺.

¹H NMR (400 MHz, DMSO-d₆) δ 1.69 (6H, s), 2.23 (3H, s), 3.32 (2H, t, J=6.8 Hz), 4.46 (2H, t, J=6.8 Hz), 7.79 (1H, t, J=1.6 Hz), 7.84 (2H, d, J=2.4 Hz), 7.95 (1H, s), 8.6 (1H, s).

The names SU20666-152, SP 152, and 152 all refer to the same compound having the formula:

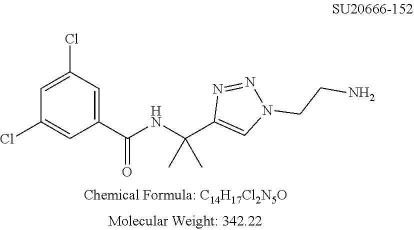 [SMILES: CC(C)(NC(=O)c1cc(Cl)cc(Cl)c1)c1cn(CCN)nn1]

Route for su20666-152

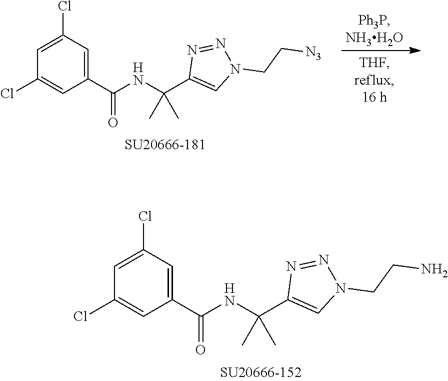

The synthesis of N-(2-(1-(2-aminoethyl)-1H-1,2,3-triazol-4-yl)propan-2-yl)-3,5-dichlorobenzamide (SU20666-152).

To a solution of compound SU20666-181 (100 mg, 0.272 mmol) in THF (15 ml) was added Ph3P (92.33 mg, 0.352 mmol) and ammonium hydroxide (19.03 mg, 0.543 mmol). The mixture was heated to reflux for 16 h. After the consumption of starting material (by LCMS), the mixture was concentrated under vacuum, and purified by prep-HPLC to give SU20666-152 (42 mg, yield: 45%) as a white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 98.92%, Rt=1.551 min; MS Calcd.: 341; MS Found: 342.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 97.25%, Rt=7.434 min; MS Calcd.: 341; MS Found: 342.2 [M+H]⁺.

¹H NMR (400 MHz, DMSO-d₆) δ 1.70 (6H, s), 2.94 (2H, t, J=6.0 Hz), 4.26 (2H, t, J=6.0 Hz), 7.79 (1H, t, J=2.0 Hz) , 7.83 (2H, d, J=1.6 Hz), 7.93 (1H, s), 8.58 (1H, s).

The names SU20666-153, SP 153, and 153 all refer to the same compound having the formula as shown below. The names SU20666-174, SP 174, and 174 all refer to the same compound having the formula as shown below. The names SU20666-175, SP 175, and 175 all refer to the same compound having the formula as shown below. The names SU20666-184, SP 184, and 184 all refer to the same compound having the formula as shown below

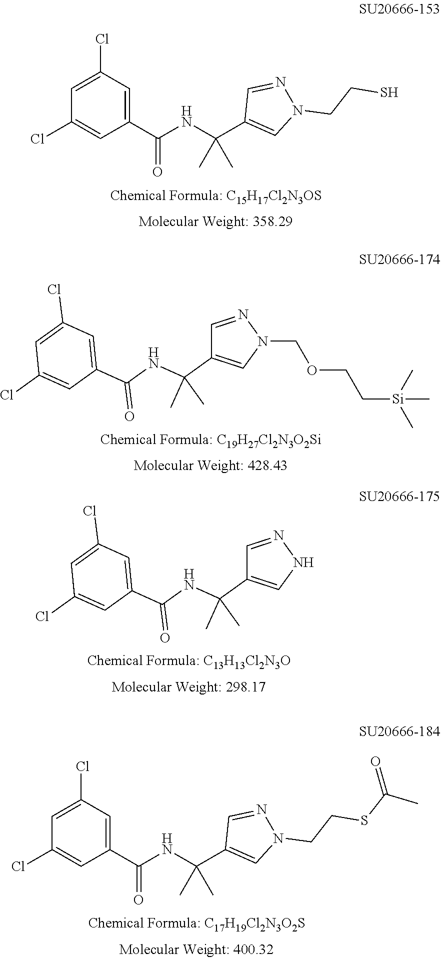 [SMILES: CC(C)(NC(=O)c1cc(Cl)cc(Cl)c1)c1cnn(CCS)c1 ; CC(C)(NC(=O)c1cc(Cl)cc(Cl)c1)c1cnn(COCC[Si](C)(C)C)c1 ; CC(C)(NC(=O)c1cc(Cl)cc(Cl)c1)c1cn[nH]c1 ; CC(=O)SCCn1cc(C(C)(C)NC(=O)c2cc(Cl)cc(Cl)c2)cn1]

Route for SU20666-153, SU20666-174, SU20666-175 and SU20666-184

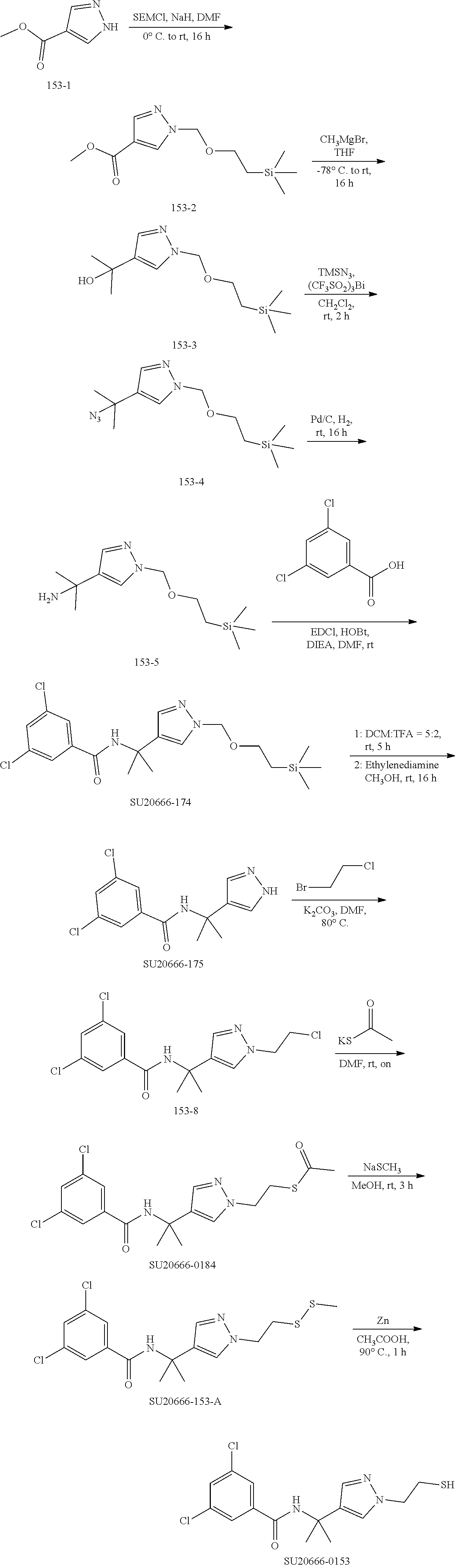

The synthesis of ethyl 1-((2-(trimethylsilyl)ethoxy)methyl)-1H-pyrazole-4-carboxylate (153-2).

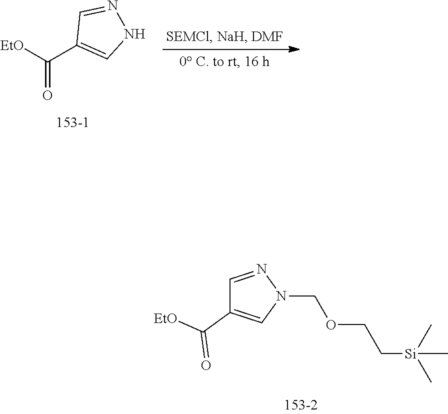 [SMILES: CCOC(=O)c1cn[nH]c1 ; CCOC(=O)c1cnn(COCC[Si](C)(C)C)c1]

To a solution of compound 153-1 (7.0 g, 50.0 mmol) in anhydrous N,N-Dimethylformamide (30 mL) was added sodium hydride (60%, 3.0 g, 75.0 mmol) in portions, the mixture was stirred for 15 min under 0° C., then, 2-(trimethylsilyl)ethoxymethyl chloride (13.3 mL, 75.0 mmol) was dropped in slowly, the reaction mixture was warmed to room temperature for overnight. After the consumption of starting material (by LCMS), the mixture was quenched with 150 mL water, and extracted with EtOAc (50×3 mL), combined the organic layer and dried over anhydrous Na₂SO₄, filtered and concentrated in vacuo to give the desired product 153-2 (13.4 g, yield: 99.3%) as colorless oil.

The synthesis of 2-(1-((2-(trimethylsilyl)ethoxy)methyl)-1H-pyrazol-4-yl)propan-2-ol (153-3).

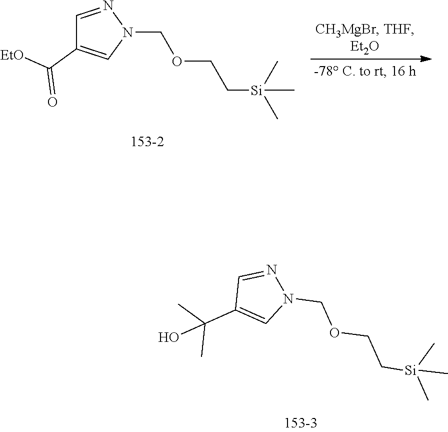 [SMILES: CCOC(=O)c1cnn(COCC[Si](C)(C)C)c1 ; CC(C)(O)c1cnn(COCC[Si](C)(C)C)c1]

To a solution of compound 153-2 (13.4 g, 49.6 mmol) in THF (130 mL) at −78° C. under N₂ protected, then CH₃MgBr (3M in Et₂O, 19.9 mL) was added slowly. The mixture was warmed to rt for stirring overnight. After the consumption of starting material (by LCMS), the mixture was quenched with 150 mL aqueous NH₄Cl, and extracted with EtOAc (100×3 mL), combined the organic layer and dried over anhydrous Na₂SO₄, filtered and concentrated in vacuo to give the desired product 153-3 (10.2 g, yield: 80%) as colorless oil.

The synthesis of 4-(2-azidopropan-2-yl)-1-((2-(trimethylsilyl)ethoxy)methyl)-1H-pyrazole (153-4).

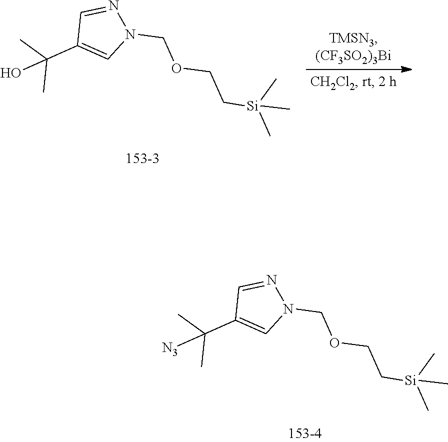 [SMILES: CC(C)(O)c1cnn(COCC[Si](C)(C)C)c1 ; CC(C)(N=[N+]=[N-])c1cnn(COCC[Si](C)(C)C)c1]

To a solution of compound 153-3 (2.9 g, 11.6 mmol) in CH₂Cl₂ (50 mL) was added TMSN₃ (1.8 mL, 13.9 mmol) and (CF₃SO₂)₃Bi (764.0 mg, 1.2 mmol). The mixture was stirred at rt for 2 h. After the consumption of starting material (by LCMS), the mixture was filtered, and the filtrate was concentrated in vacuo to give the desired product 153-4 (2.4 g, yield: 94.1%) as yellow oil.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min, Purity: 84.54%, Rt=0.867 min; MS Calcd.: 281.0; MS Found: 282.2 [M+H]⁺.

The synthesis of 2-(1-((2-(trimethylsilyl)ethoxy)methyl)-1H-pyrazol-4-yl)propan-2-amine (153-5).

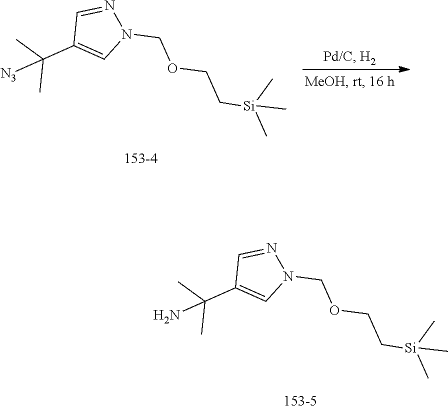 [SMILES: CC(C)(N=[N+]=[N-])c1cnn(COCC[Si](C)(C)C)c1 ; CC(C)(N)c1cnn(COCC[Si](C)(C)C)c1]

To a solution of compound 153-4 (2.8 g, 10.0 mmol) in MeOH (30 mL) was added Pd/C (10%, 618 mg). The mixture was stirred at rt for 16 h under H₂ atmosphere (1.0 atm), the mixture was filtered and concentrated in vacuo to give the desired product 153-5 (2.8 g, yield: 85.9%) as colorless oil.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min, Purity: 97.80%, Rt=0.560 min; MS Calcd.: 255.0; MS Found: 256.1 [M+H]⁺.

The synthesis of 3,5-dichloro-N-(2-(1-((2-(trimethylsilyl)ethoxy)methyl)-1H-pyrazol-4-yl)propan-2-yl)benzamide (SU20666-174).

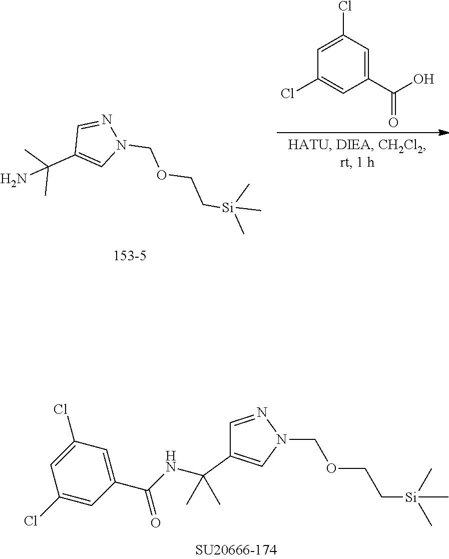 [SMILES: CC(C)(N)c1cnn(COCC[Si](C)(C)C)c1 ; O=C(O)c1cc(Cl)cc(Cl)c1 ; CC(C)(NC(=O)c1cc(Cl)cc(Cl)c1)c1cnn(COCC[Si](C)(C)C)c1]

To a solution of compound 3,5-dichlorobenzoic acid (950 mg, 5.0 mmol) in CH₂Cl₂ (25 mL) was added HATU (2.85 g, 7.5 mmol), DIEA (3.31 mL, 20.0 mmol) and 153-5 (1.28 g, 5.0 mmol). The mixture was stirred at rt for 1 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 174 (1.6 g, yield: 78.7%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 97.70%, Rt=2.460 min; MS Calcd.: 427.0; MS Found: 428.3 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 10 min, then under this condition for 5 min, finally changed to 95% [water+0.05% NH₄HCO₃] and 5% [CH₃CN+0.05% NH₄HCO₃] in 0.1 min and under this condition for 5 min), Purity: 97.93%, Rt=11.472 min.

¹H NMR (400 MHz, CDCl₃) δ 0.03 (9H, s), 0.92-0.96 (2H, m), 1.85 (6H, s), 3.59-3.64 (2H, m), 5.42 (2H, s), 6.14 (1H, s), 7.48 (1H, t, J=2.0 Hz), 7.57-7.59 (3H, m), 7.64 (1H, s).

The synthesis of N-(2-(1H-pyrazol-4-yl)propan-2-yl)-3,5-dichlorobenzamide (SU20666-175).

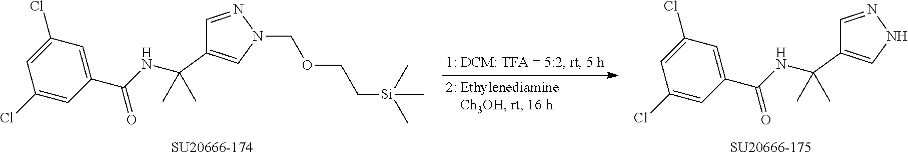 [SMILES: CC(C)(NC(=O)c1cc(Cl)cc(Cl)c1)c1cnn(COCC[Si](C)(C)C)c1 ; CC(C)(NC(=O)c1cc(Cl)cc(Cl)c1)c1cn[nH]c1]

To a solution of compound 174 (2.17 g, 5.0 mmol) in CH₂Cl₂ (20 mL) was added TFA (8 mL). The mixture was stirred at rt for 5 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo. The crude was dissolved in CH₃OH (20 mL) was added ethylenediamine (1 mL, 15.0 mmol). The mixture was stirred at rt for 16 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 175 (1.2 g, yield: 80.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 97.87%, Rt=1.908 min; MS Calcd.: 297.0; MS Found: 298.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 10 min, then under this condition for 5 min, finally changed to 95% [water+0.05% NH₄HCO₃] and 5% [CH₃CN+0.05% NH₄HCO₃] in 0.1 min and under this condition for 5 min), Purity: 97.95%, Rt=8.676 min.

¹H NMR (400 MHz, DMSO-d₆) δ 1.67 (6H, s), 7.43 (1H, s), 7.61 (1H, s), 7.76-7.77 (1H, m), 7.82 (2H, d, J=2.0 Hz), 8.36 (1H, s), 12.54 (1H, s).

The synthesis of 3,5-dichloro-N-(2-(1-(2-chloroethyl)-1H-pyrazol-4-yl)propan-2-yl)benzamide (153-8).

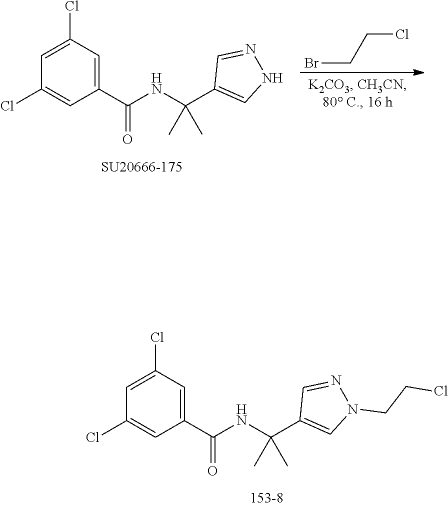

To a solution of compound 175 (290.0 mg, 1.0 mmol) in CH₃CN (5 mL) was added 1-bromo-2-chloroethane (215.0 mg, 1.5 mmol) and K₂CO₃ (652 mg, 2.0 mmol). The mixture was stirred at 80° C. for 16 h. After the consumption of starting material (by LCMS), the solvent was remove in vacuo, the crude was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 153-8 (312.0 mg, yield: 87.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min), Purity: 69.96%, Rt=0.767 min; MS Calcd.: 359.0; MS Found: 360.0 [M+H]⁺.

The synthesis of S-2-(4-(2-(3,5-dichlorobenzamido)propan-2-yl)-1H-pyrazol-1-yl)ethyl ethanethioate (SU20666-184).

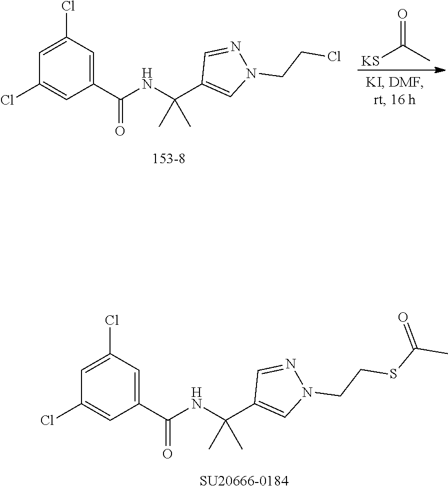

To a solution of compound 153-8 (312 mg, 0.87 mmol) in DMF (5 mL) was added Potassium iodide (29 mg, 0.20 mmol) and Potassium thioacetate (150 mg, 1.30 mmol). The mixture was stirred at rt for 16 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by flash-chromatography to give the desired product 184 (192.0 mg, yield: 48.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 96.86%, Rt=1.900 min; MS Calcd.: 399.0; MS Found: 399.9 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 98.49%, Rt=9.685 min.

¹H NMR (400 MHz, CDCl₃) δ 1.83 (6H, s), 2.37 (3H, s), 3.33 (2H, t, J=6.8 Hz), 4.27 (2H, t, J=6.4 Hz), 6.15 (1H, s), 7.47-7.50 (2H, m), 7.55 (1H, s), 7.58 (2H, d, J=2.0 Hz).

The synthesis of 3,5-dichloro-N-(2-(1-(2-(methyldisulfanyl)ethyl)-1H-pyrazol-4-yl)propan-2-yl)benzamide (SU20666-153-A).

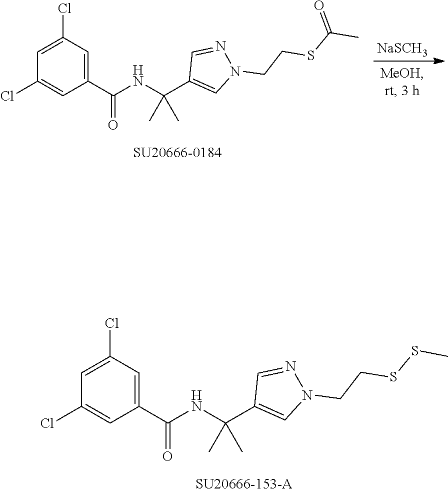

To a solution of compound 184 (120.0 mg, 0.3 mmol) in CH₃OH (5 mL) was added Sodium thiomethoxide (32.0 mg, 0.5 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo to give the product 153-A (46.0 mg, yield: 38.0%) as a white solid.

The synthesis of 3,5-dichloro-N-(2-(1-(2-mercaptoethyl)-1H-pyrazol-4-yl)propan-2-yl)benzamide (SU20666-153).

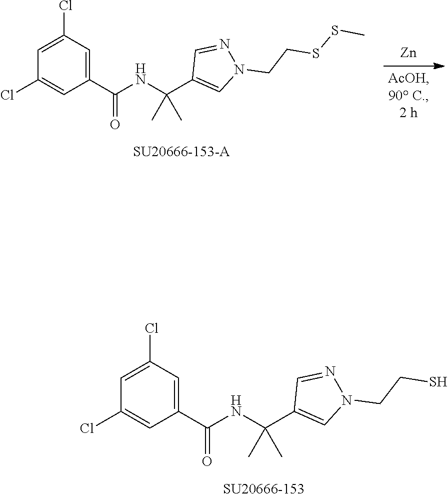

To a solution of compound 153-A (46.0 mg, 0.11 mmol) in acetic acid glacial (2 mL) was added zinc powder (23.0 mg, 0.34 mmol). The mixture was stirred at 90° C. for 1 h. After the consumption of starting material (by LCMS), the solvent was remove in vacuo, the crude was quenched with 20 mL water, and extracted with CH₂Cl₂ (20×3 mL), combined the organic layer and dried over anhydrous Na₂SO₄, filtered and concentrated in vacuo, the crude was purified by Prep-HPLC to give the product 153 (12.0 mg, yield: 27.2%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 76.40%, Rt=1.874 min; MS Calcd.: 357.0; MS Found: 357.9 [M+H]⁺.

¹H NMR (400 MHz, CDCl₃) δ 1.30 (1H, t, J=8.8 Hz), 1.74 (6H, s), 2.89-2.94 (2H, m), 4.20 (2H, t, J=6.4 Hz), 6.06 (1H, s), 7.39 (1H, t, J=1.6 Hz), 7.45 (1H, s), 7.48 (3H, t, J=1.6 Hz).

The names SU20666-154, SP 154, and 154 all refer to the same compound having the formula:

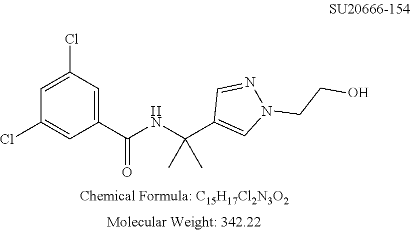 [SMILES: CC(C)(NC(=O)c1cc(Cl)cc(Cl)c1)c1cnn(CCO)c1]

Route for SU20666-154

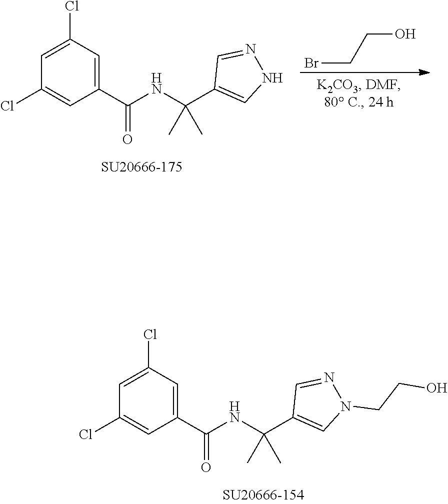

The synthesis of 3,5-dichloro-N-(2-(1-(2-hydroxyethyl)-1H-pyrazol-4-yl)propan-2-yl)benzamide (SU20666-154).

To a solution of compound 175 (595.0 mg, 2.0 mmol) in DMF (10 mL) was added 2-bromoethanol (500.0 mg, 4.0 mmol) and K₂CO₃ (830.0 mg, 6.0 mmol). The mixture was stirred at 80° C. for 24 h. After the reaction was finished (by LCMS), the reaction was quenched with 100 mL water, and extracted with EtOAc (30×3 mL), combined the organic layer and dried over anhydrous Na₂SO₄, filtered and concentrated in vacuo, the crude was purified by flash-chromatography to give the desired product 154 (250 mg, yield: 37%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 99.76%, Rt=1.645 min; MS Calcd.: 341.0; MS Found: 342.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 99.24%, Rt=8.105 min.

¹H NMR (400 MHz, DMSO-d₆) δ 1.65 (6H, s), 3.68-3.73 (2H, s), 4.06 (2H, t, J=6.0 Hz), 4.86 (1H, t, J=5.2 Hz), 7.37(1H, s), 7.60 (1H, s), 7.77-7.78 (1H, m), 7.82 (2H, d, J=1.6 Hz), 8.37 (1H, s).

The names SU20666-155, SP 155, and 155 all refer to the same compound having the formula:S

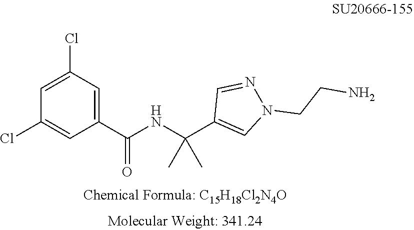 [SMILES: CC(C)(NC(=O)c1cc(Cl)cc(Cl)c1)c1cnn(CCN)c1]

Route for SU20666-155

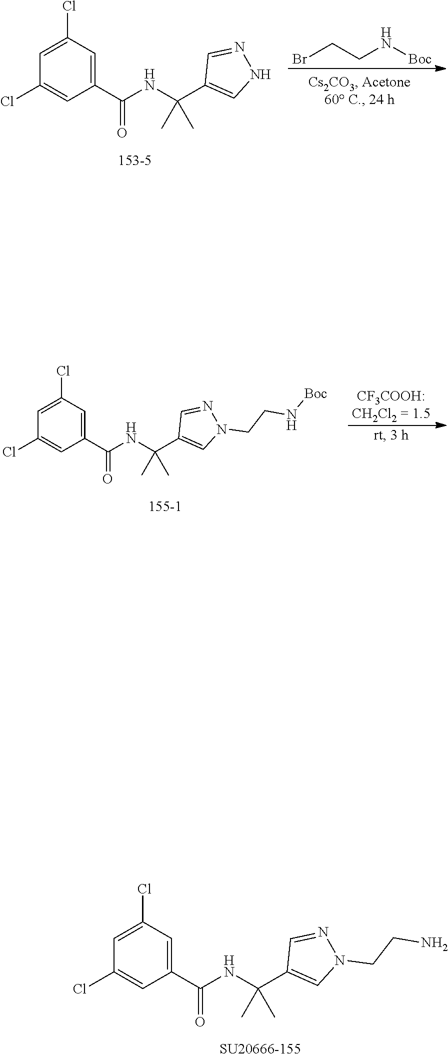

The synthesis of tert-butyl 2-(4-(2-(3,5-dichlorobenzamido)propan-2-yl)-1H-pyrazol-1-yl)ethylcarbamate (155-1).

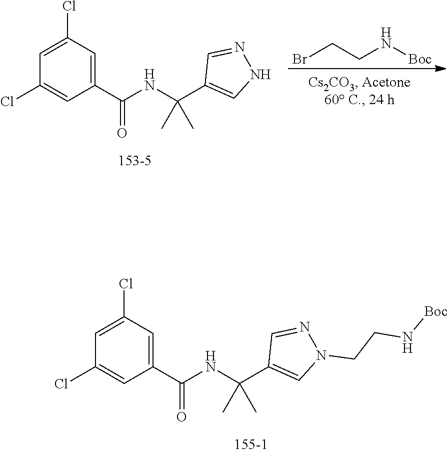 [SMILES: CC(C)(NC(=O)c1cc(Cl)cc(Cl)c1)c1cn[nH]c1 ; CC(C)(C)OC(=O)NCCBr ; CC(C)(C)OC(=O)NCCn1cc(C(C)(C)NC(=O)c2cc(Cl)cc(Cl)c2)cn1]

To a solution of compound 153-5 (208 mg, 0.7 mmol) in Acetone (10 mL) was added tert-Butyl N-(2-bromoethyl)carbamate (235 mg, 1.1 mmol) and cesiumcabonate (685 mg, 2.1 mmol). The mixture was stirred at 60° C. for 24 h. After the reaction was finished (by LCMS), the reaction was quenched with 30 mL water, and extracted with CH₂Cl₂ (20×3 mL), combined the organic layer and dried over anhydrous Na₂SO₄, filtered and concentrated in vacuo, the crude was purified by flash-chromatography to give the desired product 155-1 (221.0 mg, yield: 72.0%) as a white solid.

The synthesis of N-(2-(1-(2-aminoethyl)-1H-pyrazol-4-yl)propan-2-yl)-3,5-dichlorobenzamide (SU20666-155).

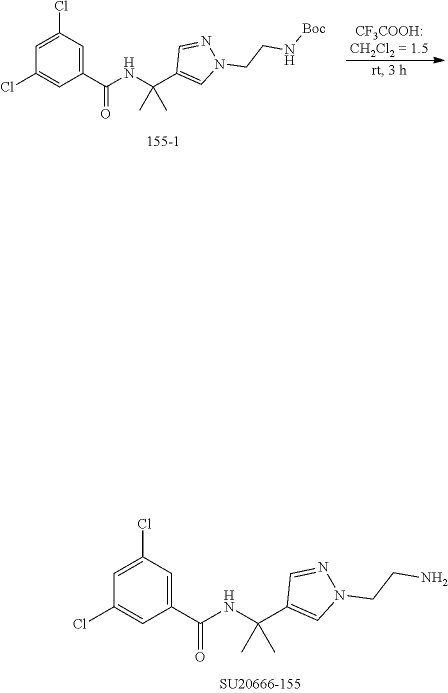

To a solution of compound 155-1 (221 mg, 0.5 mmol) in CH₂Cl₂ (5 mL) was added Trifluoroacetic acid (1 mL). The mixture was stirred at rt for 3 h. After the consumption of starting material (by LCMS), the solvent was removed in vacuo, the crude was dissolved with 20 mL water, and Potassium carbonate was added to adjust pH=9-10. Then, the aqueous phase was extracted with CH₂Cl₂ (20×3 mL), combined the organic layer and dried over anhydrous Na₂SO₄, filtered and concentrated in vacuo, the crude was dissolved with DMF and purified by Prep-HPLC to give the desired product 155 (54 mg, yield: 23%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 99.74%, Rt=1.475 min; MS Calcd.: 340.0; MS Found: 341.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 10 min, then under this condition for 5 min, finally changed to 95% [water+0.05% NH₄HCO₃] and 5% [CH₃CN+0.05% NH₄HCO₃] in 0.1 min and under this condition for 5 min), Purity: 99.15%, Rt=6.232 min.

¹H NMR (400 MHz, DMSO-d₆ +D₂O) 6 1.66 (6H, s), 2.85 (1H, t, J=6.4 Hz), 3.29 (1H, t, J=6.4 Hz), 3.99-4.07 (2H, m), 7.39 (1H, d, J=2.4 Hz), 7.61 (1H, s), 7.77 (1H, s), 7.81 (2H, d, J=1.6 Hz).

The names SU20666-156, SP 156, and 156 all refer to the same compound having the formula as shown below. The names SU20666-169, SP 169, and 169 all refer to the same compound having the formula as shown below. The names SU20666-173, SP 173, and 173 all refer to the same compound having the formula as shown below.

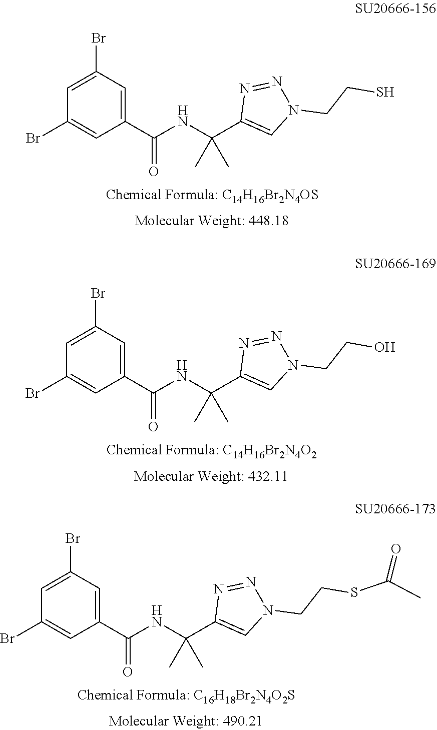 [SMILES: CC(C)(NC(=O)c1cc(Br)cc(Br)c1)c1cn(CCS)nn1 ; CC(C)(NC(=O)c1cc(Br)cc(Br)c1)c1cn(CCO)nn1 ; CC(=O)SCCn1cc(C(C)(C)NC(=O)c2cc(Br)cc(Br)c2)nn1]

Route for SU20666-156, SU20666-169 and SU20666-173

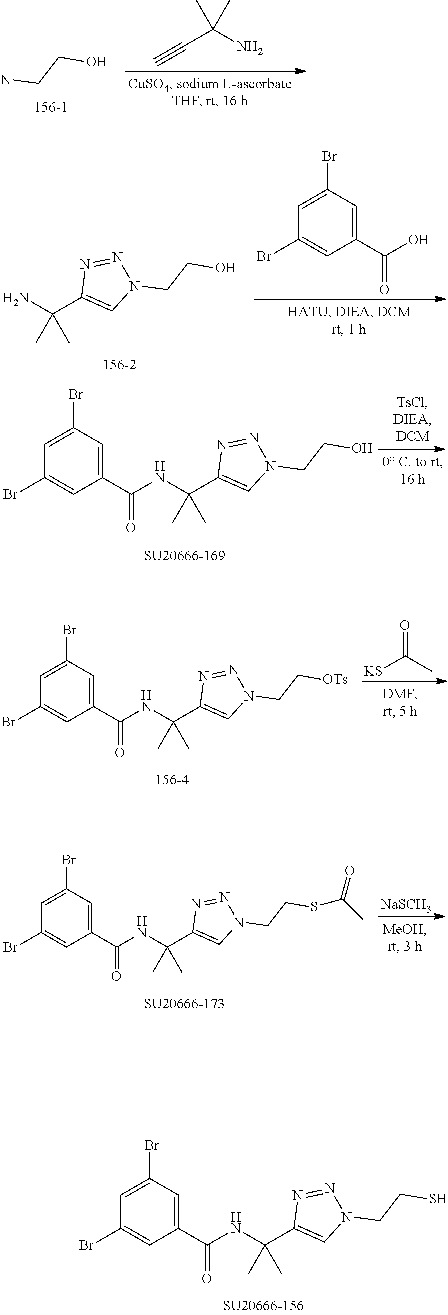

The synthesis of 2-(4-(2-aminopropan-2-yl)-1H-1,2,3-triazol-1-yl)ethanol (156-2).

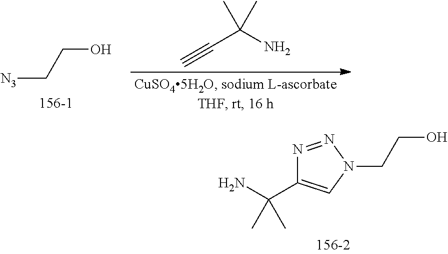

To a solution of compound 156-1 (5.0 g, 57.4 mmol)) in THF (150 mL) was added Copper sulfate pentahydrate (14.3 g, 57.4 mmol), sodium L-ascorbate (11.4 g, 57.4 mmol) and 2-methyl-3-butyn-2-amine (5.7 g, 68.9 mmol), the mixture was stirred for room temperature for overnight. After the consumption of starting material (by LCMS), the mixture was filtered and the filtrate was concentrated to give the desired product 156-2 (5.3 g, yield: 54.2%) as brown oil.

The synthesis of 3,5-dibromo-N-(2-(1-(2-hydroxyethyl)-1H-1,2,3-triazol-4-yl)propan-2-yl)benzamide (SU20666-169).

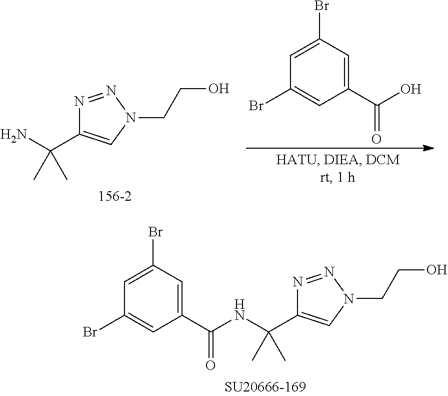 [SMILES: CC(C)(N)c1cn(CCO)nn1 ; O=C(O)c1cc(Br)cc(Br)c1 ; CC(C)(NC(=O)c1cc(Br)cc(Br)c1)c1cn(CCO)nn1]

To a solution of compound 3,5-dibromobenzoic acid (500 mg, 1.8 mmol) in CH₂Cl₂ (25 mL) was added HATU (1.0 g, 2.7 mmol), DIEA (1.1 mL, 7.2 mmol) and 156-2 (307 mg, 1.8 mmol). The mixture was stirred at rt for 1 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 169 (650.0 mg, yield: 84.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 97.20%, Rt=1.722 min; MS Calcd.: 430.0; MS Found: 431.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 10 min, then under this condition for 5 min, finally changed to 95% [water+0.05% NH₄HCO₃] and 5% [CH₃CN+0.05% NH₄HCO₃] in 0.1 min and under this condition for 5 min), Purity: 95.50%, Rt=8.133 min.

¹H NMR (400 MHz, CDCl₃) δ 1.87 (6H, s), 2.82 (1H, s), 4.08 (2H, t, J=4.8 Hz), 4.50 (2H, t, J=4.8 Hz), 7.00 (1H, s), 7.69 (1H, s), 7.78 (1H, t, J=2.0 Hz), 7.82 (2H, d, J=2.0 Hz).

The synthesis of 2-(4-(2-(3,5-dibromobenzamido)propan-2-yl)-1H-1,2,3-triazol-1-yl)ethyl 4-methylbenzenesulfonate (156-4).

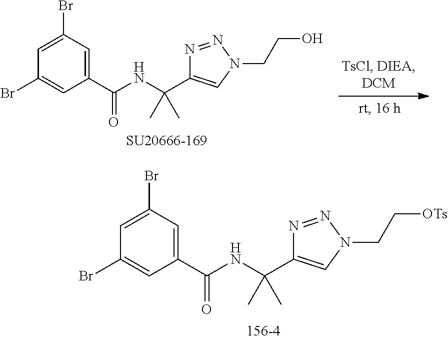 [SMILES: CC(C)(NC(=O)c1cc(Br)cc(Br)c1)c1cn(CCO)nn1 ; Cc1ccc(S(=O)(=O)OCCn2cc(C(C)(C)NC(=O)c3cc(Br)cc(Br)c3)nn2)cc1]

To a solution of compound 169 (430.0 mg, 1.0 mmol) in CH₂Cl₂ (20 mL) was added DIEA (258.5 mg, 2.0 mmol) and TsC₁ (286.0 mg, 1.5 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 156-4 (300.0 mg, yield: 51.0%) as a white solid.

The synthesis of S-2-(4-(2-(3,5-dibromobenzamido)propan-2-yl)-1H-1,2,3-triazol-1-yl)ethyl ethanethioate (SU20666-173).

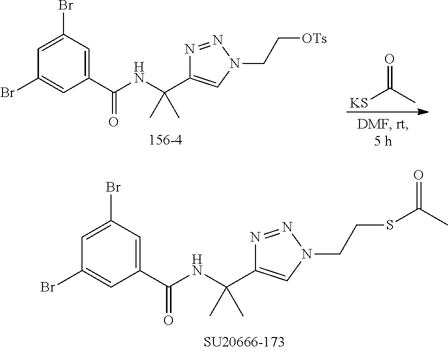

To a solution of compound 156-4 (300.0 mg, 0.54 mmol) in DMF (5 mL) was added Potassium thioacetate (89.0 mg, 0.78 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by flash-chromatography to give the desired product 173 (250.0 mg, yield: 95.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 100.00%, Rt=2.044 min; MS Calcd.: 488.0; MS Found: 489.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 91.33%, Rt=9.547 min.

¹H NMR (400 MHz, DMSO-d₆) δ 1.68 (6H, s), 2.33 (3H, s), 3.32 (2H, t, J=1.6 Hz), 4.46 (2H, t, J=1.6 Hz), 7.95(1H, s), 8.00-8.01 (3H, m), 8.60 (1H, s).

The synthesis of 3,5-dibromo-N-(2-(1-(2-mercaptoethyl)-1H-1,2,3-triazol-4-yl)propan-2-yl)benzamide (SU20666-156).

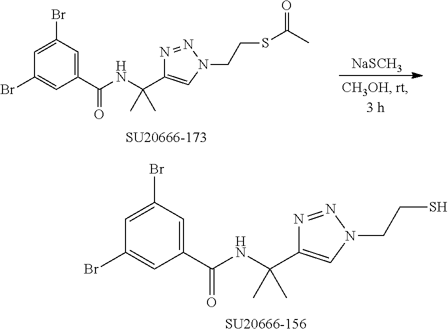

To a solution of compound 173 (150.0 mg, 0.31 mmol) in CH₃OH (5 mL) was added Sodium thiomethoxide (33.0 mg, 0.46 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo, the crude was purified by prep-HPLC to give the desired product 156 (55.0 mg, yield: 40.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.832 min; MS Calcd.: 446.0; MS Found: 446.8 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 100.00%, Rt=9.384 min.

¹H NMR (400 MHz, CDCl₃) δ 1.41 (1H, t, J=8.8 Hz), 1.82 (6H, s), 2.98 (2H, t, J=6.4 Hz), 4.48 (2H, t, J=6.0 Hz), 6.86 (1H, s), 7.58 (1H, s), 7.70 (1H, s), 7.75 (2H, d, J=1.2 Hz).

The names SU20666-157, SP 157, and 157 all refer to the same compound having the formula as shown below. The names SU20666-170, SP 170, and 170 all refer to the same compound having the formula as shown below. The names SU20666-171, SP 171, and 171 all refer to the same compound having the formula as shown below.

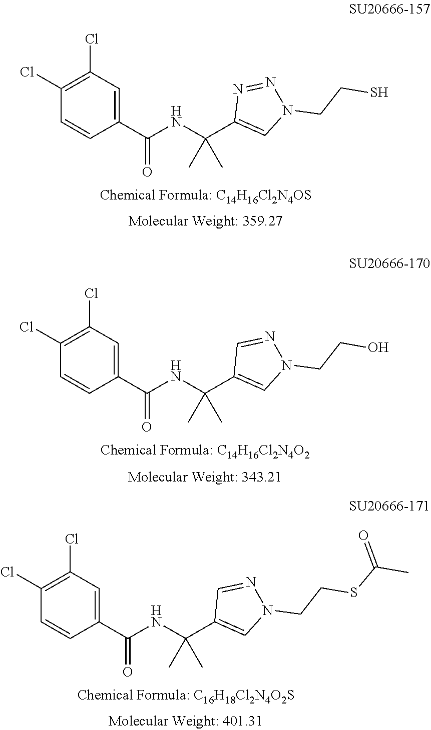 [SMILES: CC(C)(NC(=O)c1ccc(Cl)c(Cl)c1)c1cn(CCS)nn1 ; CC(C)(NC(=O)c1ccc(Cl)c(Cl)c1)c1cnn(CCO)c1 ; CC(=O)SCCn1cc(C(C)(C)NC(=O)c2ccc(Cl)c(Cl)c2)cn1]

Route for SU20666-157, SU20666-170 and SU20666-171

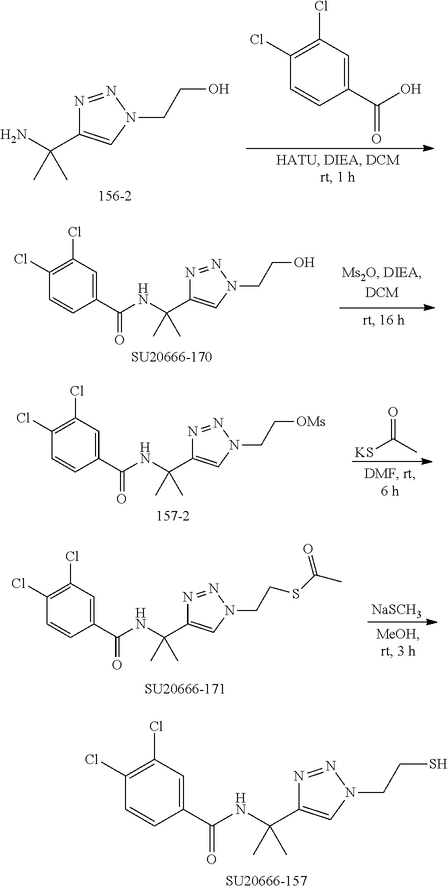

The synthesis of 3,4-dichloro-N-(2-(1-(2-hydroxyethyl)-1H-1,2,3-triazol-4-yl)propan-2-yl)benzamide (SU20666-170).

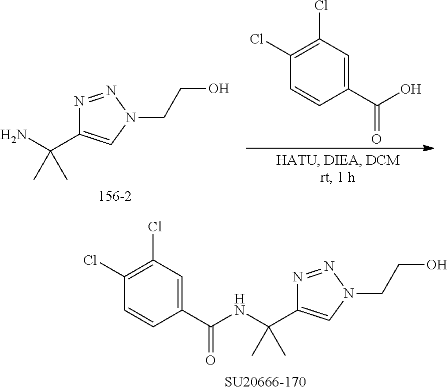 [SMILES: CC(C)(N)c1cn(CCO)nn1 ; O=C(O)c1ccc(Cl)c(Cl)c1 ; CC(C)(NC(=O)c1ccc(Cl)c(Cl)c1)c1cn(CCO)nn1]

To a solution of compound 3,4-dichlorobenzoic acid (570.0 mg, 3.0 mmol) in CH₂Cl₂ (25 mL) was added HATU (1.7 g, 4.5 mmol), DIEA (2.0 mL, 12.0 mmol) and 156-2 (511 mg, 3.0 mmol). The mixture was stirred at rt for 1 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 170 (650.0 mg, yield: 64.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 97.53%, Rt=1.595 min; MS Calcd.: 342.0; MS Found: 343.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 99.15%, Rt=7.524 min.

¹H NMR (400 MHz, DMSO-d₆) δ 1.70 (6H, s), 3.77 (2H, q, J=5.2 Hz), 4.34 (2H, t, J=5.6 Hz), 5.01 (1H, t, J=5.6 Hz), 7.71-7.74 (1H, m), 7.77-7.80 (1H, m), 7.89 (1H, s), 8.08 (1H, d, J=2.0 Hz), 8.53 (1H, s).

The synthesis of 2-(4-(2-(3,4-dichlorobenzamido)propan-2-yl)-1H-1,2,3-triazol-1-yl)ethyl methanesulfonate (157-2).

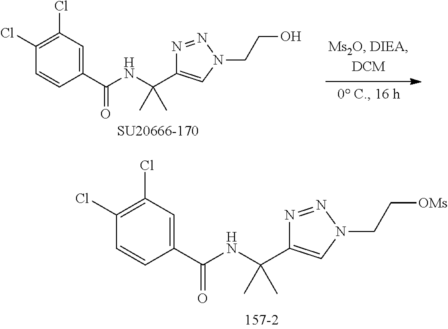 [SMILES: CC(C)(NC(=O)c1ccc(Cl)c(Cl)c1)c1cn(CCO)nn1 ; CC(C)(NC(=O)c1ccc(Cl)c(Cl)c1)c1cn(CCOS(C)(=O)=O)nn1]

To a solution of compound 170 (457.0 mg, 1.3 mmol) in CH₂Cl₂ (20 mL) was added DIEA (345.0 mg, 2.7 mmol) and Ms₂O (349.0 mg, 2.0 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 157-2 (520.0 mg, yield: 92.4%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min, Purity: 57.88%, Rt=0.666 min; MS Calcd.: 420.0; MS Found: 420.9 [M+H]⁺.

The synthesis of S-2-(4-(2-(3,4-dichlorobenzamido)propan-2-yl)-1H-1,2,3-triazol-1-yl)ethyl ethanethioate (SU20666-171).

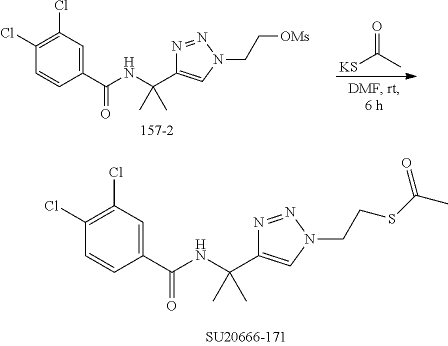

To a solution of compound 157-2 (572.0 mg, 1.34 mmol) in DMF (5 mL) was added Potassium thioacetate (230.0 mg, 2.01 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by Prep-HPLC to give the desired product 171 (218.0 mg, yield: 38.8%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 100.00%, Rt=1.925 min; MS Calcd.: 400.0; MS Found: 401.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 100.00%, Rt=9.057 min.

¹H NMR (400 MHz, DMSO-d₆) δ 1.69 (6H, s), 2.33 (3H, s), 3.33 (2H, t, J=6.8 Hz), 4.46 (2H, t, J=6.8 Hz), 7.72-7.74 (1H, m), 7.77-7.80 (1H, m), 7.95 (1H, s), 8.09 (1H, d, J=2.0 Hz), 8.55 (1H, s).

The synthesis of 3,4-dichloro-N-(2-(1-(2-mercaptoethyl)-1H-1,2,3-triazol-4-yl)propan-2-yl)benzamide (SU20666-157).

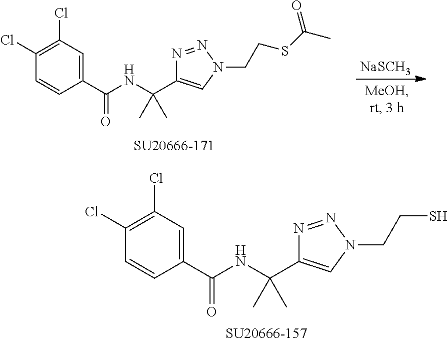

To a solution of compound 171 (150.0 mg, 0.38 mmol) in CH₃OH (5 mL) was added Sodium thiomethoxide (40.0 mg, 0.46 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo, the crude was purified by prep-HPLC to give the desired product 157 (60.0 mg, yield: 44.0%) as a white solid.

LC-MS (Agilent LCMS 1200-6110, Column: Waters X-Bridge C18 (50 mm*4.6 mm*3.5μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.748 min; MS Calcd.: 358.0; MS Found: 359.0 [M+H]⁺.

HPLC (Agilent HPLC 1200; Column: L-column2 ODS (150 mm*4.6 mm*5.0μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 97.89%, Rt=8.852 min.

¹H NMR (400 MHz, DMSO-d₆) δ 1.70 (6H, s), 2.43 (1H, s), 2.94 (2H, t, J=6.8 Hz), 4.46 (2H, t, J=6.8 Hz), 7.72-7.74 (1H, m), 7.77-7.80 (1H, m), 7.97 (1H, s), 8.08 (1H, d, J=2.0 Hz), 8.55 (1H, s).

The names SU20666-158, SP 158, and 158 all refer to the same compound having the formula as shown below. The names SU20666-176, SP 176, and 176 all refer to the same compound having the formula as shown below. The names SU20666-177, SP 177, and 177 all refer to the same compound having the formula as shown below.

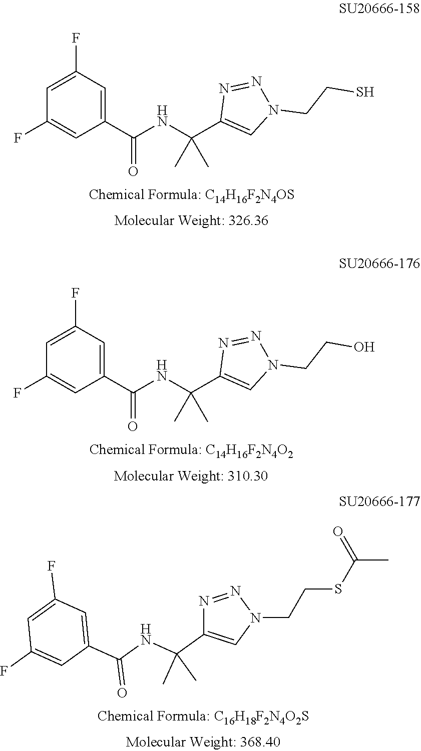 [SMILES: CC(C)(NC(=O)c1cc(F)cc(F)c1)c1cn(CCS)nn1 ; CC(C)(NC(=O)c1cc(F)cc(F)c1)c1cn(CCO)nn1 ; CC(=O)SCCn1cc(C(C)(C)NC(=O)c2cc(F)cc(F)c2)nn1]

Route for SU20666-158, SU20666-176 and SU20666-177

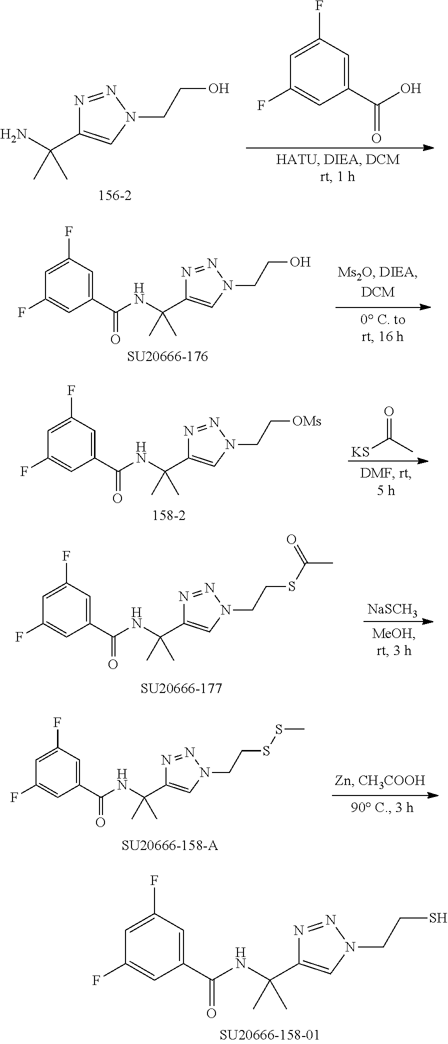

The synthesis of 3,5-difluoro-N-(2-(1-(2-hydroxyethyl)-1H-1,2,3-triazol-4-yl)propan-2-yl)benzamide (SU20666-176).

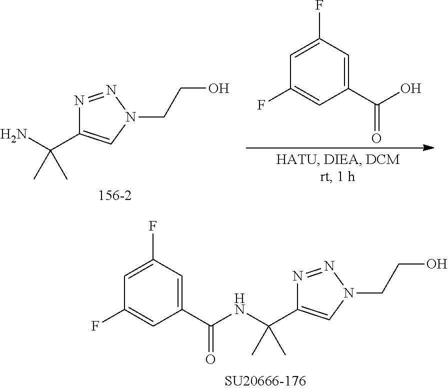 [SMILES: CC(C)(N)c1cn(CCO)nn1 ; O=C(O)c1cc(F)cc(F)c1 ; CC(C)(NC(=O)c1cc(F)cc(F)c1)c1cn(CCO)nn1]

To a solution of compound 3,5-difluorobenzoic acid (237.0 mg, 1.5 mmol) in CH₂Cl₂ (15 mL) was added HATU (856 mg, 2.3 mmol), DIEA (1.0 mL, 6.0 mmol) and 156-2 (256.0 mg, 1.5 mmol). The mixture was stirred at rt for 1 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 176 (400.0 mg, yield: 86.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 100.00%, Rt=1.410 min; MS Calcd.: 310.0; MS Found: 311.1 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 98.69%, Rt=6.539 min.

¹H NMR (400 MHz, DMSO-d₆) δ 1.70 (6H, s), 3.78 (2H, q, J=5.2 Hz), 4.34 (2H, t, J=5.6 Hz), 5.01 (1H, t, J=5.2 Hz), 7.41-7.47 (1H, m), 7.52-7.57 (2H, m), 7.89 (1H, s), 8.49 (1H, s).

The synthesis of 2-(4-(2-(3,5-difluorobenzamido)propan-2-yl)-1H-1,2,3-triazol-1-yl)ethyl methanesulfonate (158-2).

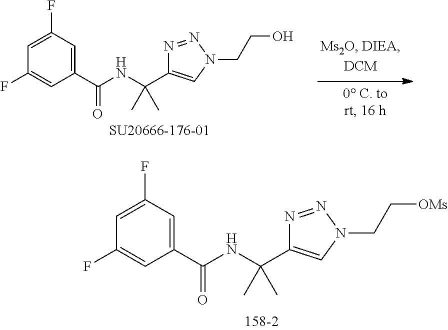 [SMILES: CC(C)(NC(=O)c1cc(F)cc(F)c1)c1cn(CCO)nn1 ; CC(C)(NC(=O)c1cc(F)cc(F)c1)c1cn(CCOS(C)(=O)=O)nn1]

To a solution of compound 176 (310.0 mg, 1.0 mmol) in CH₂Cl₂ (10 mL) was added DIEA (259.0 mg, 2.0 mmol) and Ms₂O (262.0 mg, 1.5 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 158-2 (369.0 mg, yield: 95.0%) as a white solid.

The synthesis of S-2-(4-(2-(3,5-difluorobenzamido)propan-2-yl)-1H-1,2,3-triazol-1-yl)ethyl ethanethioate (SU20666-177).

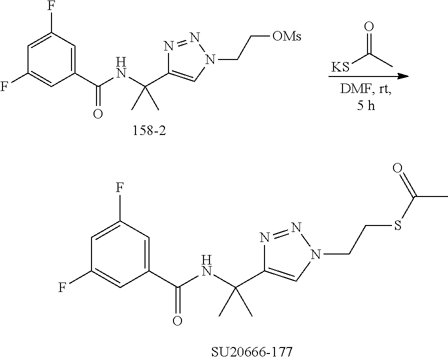

To a solution of compound 158-2 (388.0 mg, 1.0 mmol) in DMF (5 mL) was added Potassium thioacetate (114.0 mg, 1.5 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by prep-HPLC to give the desired product 177 (226.0 mg, yield: 61.4%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.653 min; MS Calcd.: 368.0; MS Found: 369.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 100.00%, Rt=8.204 min.

¹H NMR (400 MHz, CDCl₃) δ 1.90 (6H, s), 2.40 (3H, s), 3.39 (2H, t, J=6.8 Hz), 5.40 (2H, t, J=6.8 Hz), 6.92-6.97 (1H, m), 7.04 (1H, s), 7.29-7.33 (2H, m), 7.62 (1H, s).

The synthesis of 3,5-difluoro-N-(2-(1-(2-(methyldisulfanyl)ethyl)-1H-1,2,3-triazol-4-yl)propan-2-yl)benzamide (SU20666-158-A).

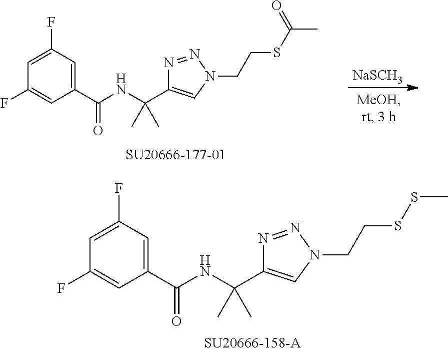 [SMILES: CC(=O)SCCn1cc(C(C)(C)NC(=O)c2cc(F)cc(F)c2)nn1 ; "NaSCH3" ; CSSCCn1cc(C(C)(C)NC(=O)c2cc(F)cc(F)c2)nn1]

To a solution of compound 177 (200.0 mg, 0.54 mmol) in CH₃OH (5 mL) was added Sodium thiomethoxide (57.0 mg, 0.81 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo to give the product 158-A (200.0 mg, yield: 99.0%) as a white solid.

The synthesis of 3,5-difluoro-N-(2-(1-(2-mercaptoethyl)-1H-1,2,3-triazol-4-yl)propan-2-yl)benzamide (SU20666-158).

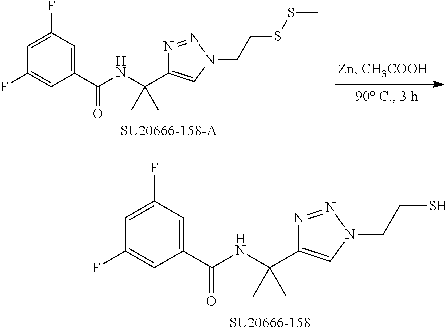 [SMILES: CSSCCn1cc(C(C)(C)NC(=O)c2cc(F)cc(F)c2)nn1 ; CC(C)(NC(=O)c1cc(F)cc(F)c1)c1cn(CCS)nn1]

To a solution of compound 158-A (200.0 mg, 0.54 mmol) in acetic acid glacial (5 mL) was added zinc powder (53.0 mg, 0.81 mmol). The mixture was stirred at 90° C. for 1 h. After the consumption of starting material (by LCMS), the solvent was remove in vacuo, the crude was quenched with 20 mL water, and extracted with CH₂Cl₂ (20×3 mL), combined the organic layer and dried over anhydrous Na₂SO₄, filtered and concentrated in vacuo, the crude was purified by Prep-HPLC to give the product 158 (60.0 mg, yield: 34.1%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.619 min; MS Calcd.: 326.0; MS Found: 327.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 98.68%, Rt=7.961 min.

¹H NMR (400 MHz, DMSO-d₆) δ 1.70 (6H, s), 2.44 (1H, t, J=8.0 Hz), 2.95 (2H, q, J=6.8 Hz), 4.46 (2H, t, J=6.8 Hz), 7.41-7.47 (1H, m), 7.52-7.56 (2H, m), 7.98 (1H, s), 8.51 (1H, s).

The names SU20666-159, SP 159, and 159 all refer to the same compound having the formula:

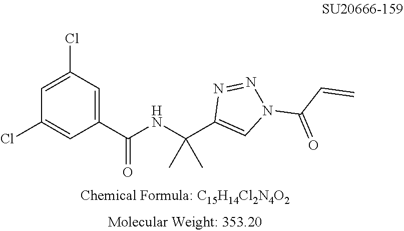 [SMILES: C=CC(=O)n1cc(C(C)(C)NC(=O)c2cc(Cl)cc(Cl)c2)nn1]

Route for SU20666-159

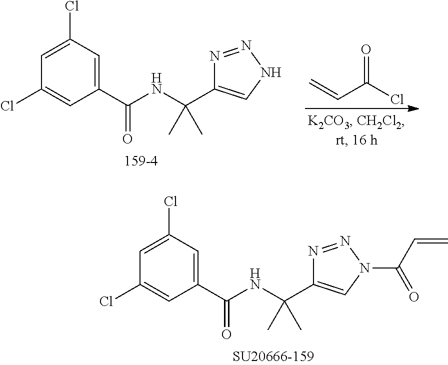

The synthesis of N-(2-(1-acryloyl-1H-1,2,3-triazol-4-yl)propan-2-yl)-3,5-dichlorobenzamide (SU20666-159).

To a solution of compound 159-4 (209.0 mg, 0.7 mmol) in dry CH₂Cl₂ (5 mL) was added K₂CO₃ (291.0 mg, 2.1 mmol) and Acrylyl chloride (95.0 mg, 1.1 mmol). The mixture was stirred at rt for 16 h. After the consumption of starting material (by LCMS), the reaction mixture was quenched with 20 mL water, and extracted with CH₂Cl₂ (20×3 mL), combined the organic layer and dried over anhydrous Na₂SO₄, filtered and concentrated in vacuo, the crude was purified by flash-chormatography and recrystallization from petroleum ether/ethyl acetate (1:1) to give the desired product 159 (150.0 mg, yield: 61.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 97.64%, Rt=1.940 min; MS Calcd.: 352.0; MS Found: 352.9 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA ] in 0.1 min and under this condition for 5 min), Purity: 93.78%, Rt=9.533 min.

¹H NMR (400 MHz, DMSO-d₆) δ 1.74 (6H, s), 6.36-6.39 (1H, m), 6.75-6.79 (1H, m), 7.48-7.55 (1H, m), 7.80-7.88 (3H, m), 8.65 (1H, s), 8.78 (1H, s).

The names SU20666-162, SP 162, and 162 all refer to the same compound having the formula:

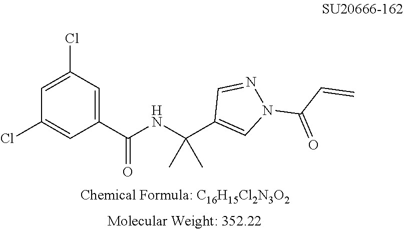 [SMILES: C=CC(=O)n1cc(C(C)(C)NC(=O)c2cc(Cl)cc(Cl)c2)cn1]

Route for SU20666-162

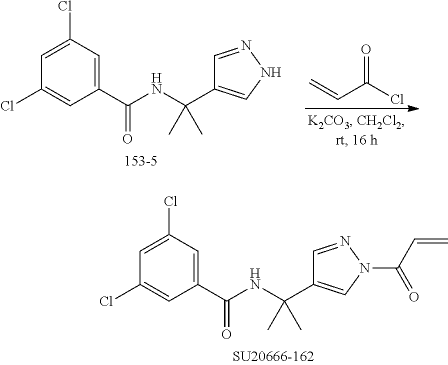

The synthesis of N-(2-(1-acryloyl-1H-pyrazol-4-yl)propan-2-yl)-3,5-dichlorobenzamide (SU20666-162).

To a solution of compound 153-5 (80.0 mg, 0.3 mmol) in dry CH₂Cl₂ (5 mL) was added K₂CO₃ (112.0 mg, 0.8 mmol) and acrylyl chloride (36.0 mg, 0.4 mmol). The mixture was stirred at rt for 16 h. After the consumption of starting material (by LCMS), the reaction mixture was quenched with 20 mL water, and extracted with CH₂Cl₂ (20×3 mL), combined the organic layer and dried over anhydrous Na₂SO₄, filtered and concentrated in vacuo, the crude was purified by flash-chormatography and recrystallization from petroleum ether/ethyl acetate (1:1) to give the desired product 162 (36.0 mg, yield: 38.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 98.42%, Rt=2.206 min; MS Calcd.:351.0; MS Found: 352.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% NH₄HCO₃] and 5% [CH₃CN+0.05% NH₄HCO₃] to 5% [water+0.05% TFA] and 95% [water+0.05% NH₄HCO₃] in 10 min, then under this condition for 5 min, finally changed to 95% [water+0.05% NH₄HCO₃] and 5% [CH₃CN+0.05% NH₄HCO₃] in 0.1 min and under this condition for 5 min), Purity: 97.18%, Rt=10.249 min.

¹H NMR (400 MHz, DMSO-d₆) δ 1.68 (6H, s), 6.18-6.21 (1H, m), 6.61-6.65 (1H, m), 7.44-7.51 (1H, m), 7.79 (1H, t, J=2.0 Hz), 7.85 (2H, d, J=2.0 Hz), 7.94 (1H, s), 8.33 (1H, s), 8.58 (1H, s).

The names SU20666-163, SP 163, and 163 all refer to the same compound having the formula:

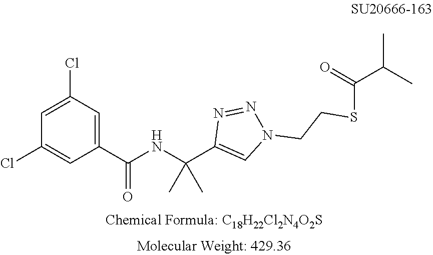 [SMILES: CC(C)C(=O)SCCn1cc(C(C)(C)NC(=O)c2cc(Cl)cc(Cl)c2)nn1]

Route for SU 20666-163

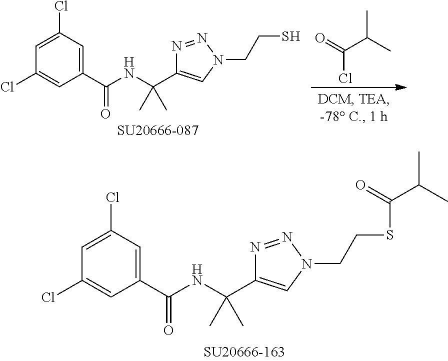

The synthesis of S-2-(4-(2-(3,5-dichlorobenzamido)propan-2-yl)-1H-1,2,3-triazol-1-yl)ethyl 2-methylpropanethioate (SU20666-163).

To a solution of compound SU20666-087 (100 mg, 0.278 mmol) in DCM (20 ml) was added TEA (71.95 mg, 0.557 mmol) and acrylyl chloride (37.75 mg, 0.417 mmol). The mixture was stirred at −78° C. for 1 h. After the consumption of starting material (by LCMS), the mixture was concentrated under vacuum and purified by prep-HPLC to give SU20666-163 (70 mg, yield: 58%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05 TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.981 min; MS Calcd.: 428; MS Found: 428.9 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 97.12%, Rt=9.780 min; MS Calcd.: 428; MS Found: 428.9 [M+H]⁺.

¹H NMR (400 MHz, DMSO-d₆) δ 1.08 (6H, d, J=6.8 Hz), 1.69 (6H, s), 2.70-2.75 (1H, m), 3.31 (2H, d, J=6.4 Hz), 4.46 (2H, t, J=6.8 Hz), 7.79 (1H, t, J=2.0 Hz), 7.84 (2H, d, J=1.6 Hz), 7.94 (1H, s), 8.60 (1H, s).

The names SU20666-164, SP 164, and 164 all refer to the same compound having the formula:

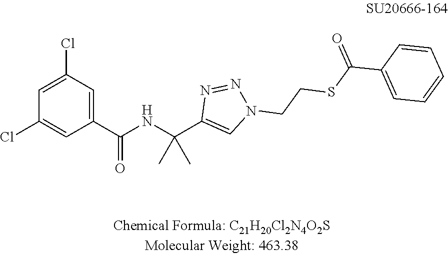 [SMILES: CC(C)(NC(=O)c1cc(Cl)cc(Cl)c1)c1cn(CCSC(=O)c2ccccc2)nn1]

Route for SU20666-164

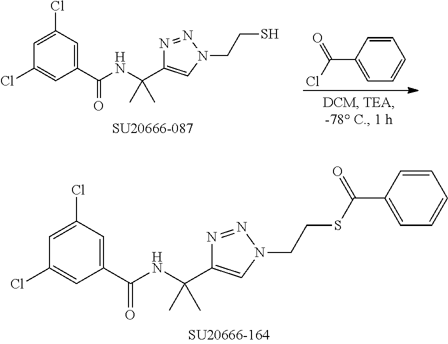 [SMILES: CC(C)(NC(=O)c1cc(Cl)cc(Cl)c1)c1cn(CCS)nn1 ; O=C(Cl)c1ccccc1 ; CC(C)(NC(=O)c1cc(Cl)cc(Cl)c1)c1cn(CCSC(=O)c2ccccc2)nn1]

The synthesis of S-2-(4-(2-(3,5-dichlorobenzamido)propan-2-yl)-1H-1,2,3-triazol-1-yl)ethyl benzothioate (SU20666-164).

To a solution of compound SU20666-087 (100 mg, 0.278 mmol) in DCM (20 ml) was added TEA (71.95 mg, 0.557 mmol) and benzoyl chloride (58.62 mg, 0.417 mmol). The mixture was stirred at −78° C. for 1 h. After the consumption of starting material (by LCMS), the mixture was concentrated under vacuum and purified by prep-HPLC to give SU20666-164 (75 mg, yield: 58.1%) as a white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 96.93%, Rt=2.228 min; MS Calcd.: 462; MS Found: 463.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 97.65%, Rt=10.013 min; MS Calcd.: 462; MS Found: 463.2 [M+H]⁺.

¹H NMR (400 MHz, DMSO-d₆) δ 1.67 (6H, s), 3.55 (2H, t, J=6.8 Hz), 4.58 (2H, t, J=6.8 Hz), 7.55 (2H, t, J=7.6 Hz) , 7.79 (1H, t, J=2.0 Hz), 7.83 (2H, d, J=1.6 Hz), 7.88-7.90 (2H, m), 8.00 (1H, s), 8.60 (1H, s).

The names SU20666-166, SP 166, and 166 all refer to the same compound having the formula as shown below. The names SU20666-182, SP 182, and 182 all refer to the same compound having the formula as shown below. The names SU20666-183, SP 183, and 183 all refer to the CAMP enmnniind having the fnrmnla ac chnwn helnw

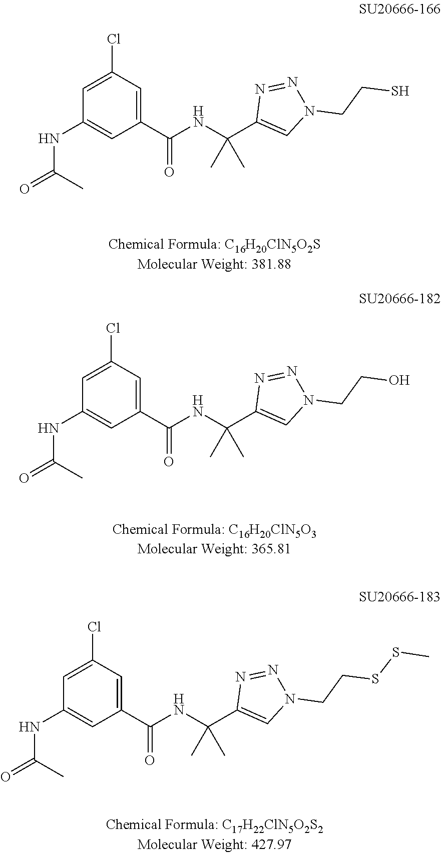 [SMILES: CC(=O)Nc1cc(Cl)cc(C(=O)NC(C)(C)c2cn(CCS)nn2)c1 ; CC(=O)Nc1cc(Cl)cc(C(=O)NC(C)(C)c2cn(CCO)nn2)c1 ; CSSCCn1cc(C(C)(C)NC(=O)c2cc(Cl)cc(NC(C)=O)c2)nn1]

Route for SU20666-166, SU20666-182 and SU20666-183

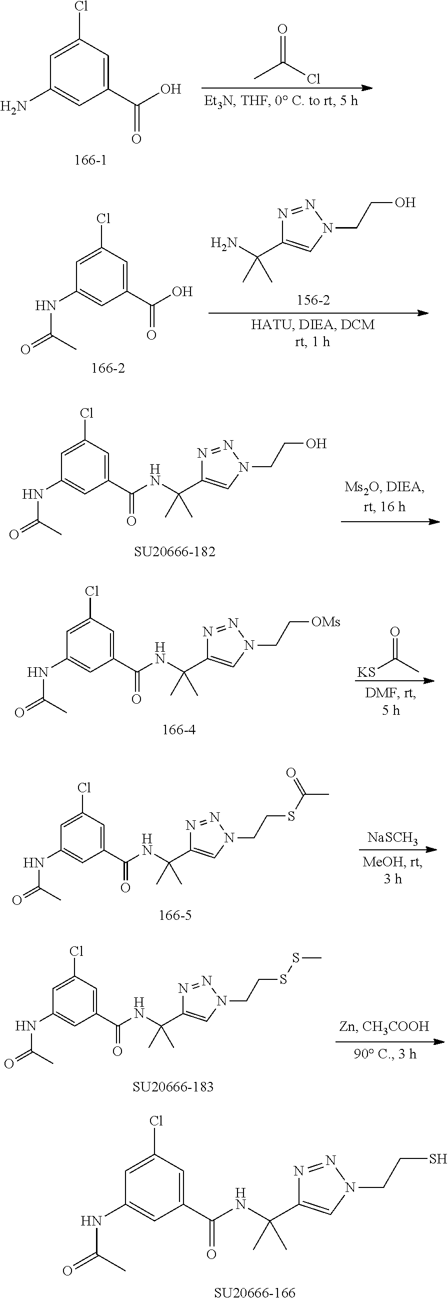

The synthesis of 3,5-difluoro-N-(2-(1-(2-hydroxyethyl)-1H-1,2,3-triazol-4-yl)propan-2-yl)benzamide (166-2).

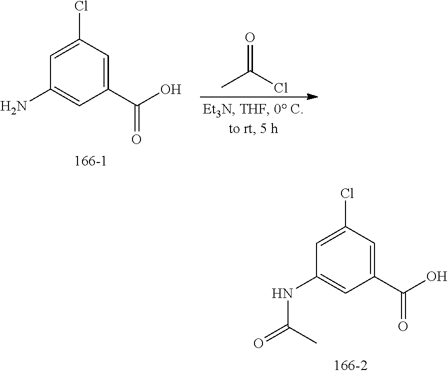

To a solution of compound 3-amino—S—Chlorobenzoic acid (342.0 mg, 2.0 mmol) in THF (15 mL) was added Et₃N (606 mg, 6.0 mmol) and acetyl chloride (157.0 mg, 2.0 mmol) at 0° C. Then, the mixture was warmed to rt for 5 h. After the consumption of starting material (by LCMS), the reaction solvent was removed in vacuo, the crude was dissolved with 30 mL water, and 1N HCl was added to adjust pH=2-3, filtered and collected the filter cake, dried over freeze-drying to give the desired product 166-2 (410.0 mg, yield: 96.2%) as a white solid.

The synthesis of 3-acetamido—S—Chloro-N-(2-(1-(2-hydroxyethyl)-1H-1,2,3-triazol-4-v1)propan-2-v1)benzamide (SU20666-182).

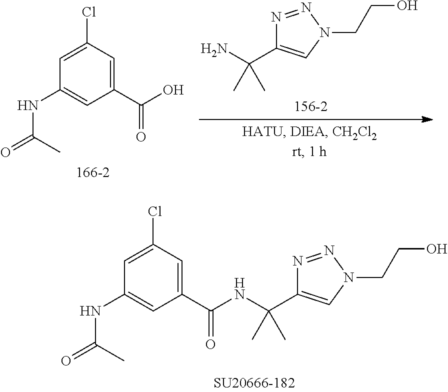 [SMILES: CC(=O)Nc1cc(Cl)cc(C(=O)O)c1 ; CC(C)(N)c1cn(CCO)nn1 ; CC(=O)Nc1cc(Cl)cc(C(=O)NC(C)(C)c2cn(CCO)nn2)c1]

To a solution of compound 3-acetamido—S—Chlorobenzoic acid (410.0 mg, 1.9 mmol) in CH₂Cl₂ (30 mL) was added HATU (1.1 g, 2.9 mmol), DIEA (1.3 mL, 7.6 mmol) and 156-2 (325.0 mg, 1.9 mmol). The mixture was stirred at rt for 1 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 182 (500.0 mg, yield: 72.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.320 min; MS Calcd.: 365.0; MS Found: 366.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 100.00%, Rt=6.109 min.

¹H NMR (400 MHz, DMSO-d₆) δ 1.69 (6H, s), 2.06 (3H, s), 3.75-3.79 (2H, m), 4.34 (2H, t, J=5.6 Hz), 5.02 (1H, t, J=5.2 Hz), 7.56 (1H, t, J=1.6 Hz), 7.75 (1H, t, J=1.6 Hz), 7.87 (1H, s), 7.97 (1H, t, J=1.6 Hz), 10.29 (1H, s).

The synthesis of 2-(4-(2-(3-acetamido—S—Chlorobenzamido)propan-2-yl)-1H-1,2,3-triazol-1-yl)ethyl methanesulfonate (166-4).

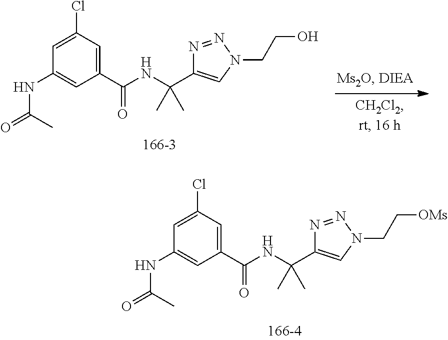 [SMILES: CC(=O)Nc1cc(Cl)cc(C(=O)NC(C)(C)c2cn(CCO)nn2)c1 ; CC(=O)Nc1cc(Cl)cc(C(=O)NC(C)(C)c2cn(CCOS(C)(=O)=O)nn2)c1]

To a solution of compound 166-3 (300.0 mg, 0.8 mmol) in CH₂Cl₂ (10 mL) was added DIEA (212.0 mg, 1.6 mmol) and Ms₂O (215.0 mg, 1.2 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 166-4 (325.0 mg, yield: 89.5%) as a white solid.

The synthesis of S-2-(4-(2-(3-acetamido—S—Chlorobenzamido)propan-2-yl)-1H-1,2,3-triazol-1-yl)ethyl ethanethioate (166-5).

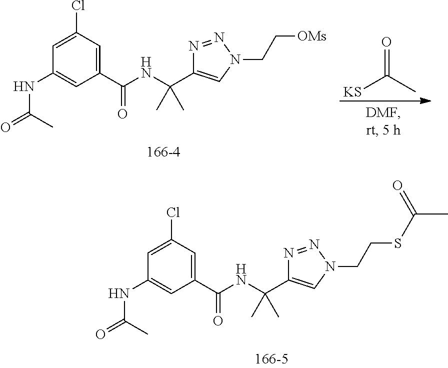

To a solution of compound 166-4 (220.0 mg, 0.50 mmol) in DMF (5 mL) was added Potassium thioacetate (86.0 mg, 0.75 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by Prep-HPLC to give the desired product 166-5 (180.0 mg, yield: 85.3%) as a white solid.

LC-MS (Agilent LCMS 1200-6110, Column: Waters X-Bridge C18 (50 mm*4.6 mm*3.5 p,m); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min), Purity: 66.22%, Rt=0.596 min; MS Calcd.: 423.0; MS Found: 424.0 [M+H]⁺.

The synthesis of 3-acetamido—S—Chloro-N-(2-(1-(2-(methyldisulfanypethyl)-1H-1,2,3-triazol-4-yl)propan-2-yl)benzamide (SU20666-183).

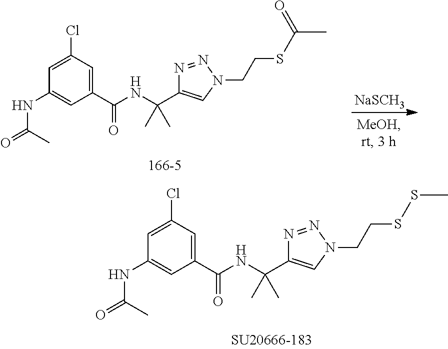 [SMILES: CC(=O)Nc1cc(Cl)cc(C(=O)NC(C)(C)c2cn(CCSC(C)=O)nn2)c1 ; "NaSCH3" ; CSSCCn1cc(C(C)(C)NC(=O)c2cc(Cl)cc(NC(C)=O)c2)nn1]

To a solution of compound 166-5 (180.0 mg, 0.43 mmol) in CH₃OH (5 mL) was added Sodium thiomethoxide (45.0 mg, 0.64 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo to give the product 183 (175.0 mg, yield: 95.3%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.772 min; MS Calcd.: 372.0; MS Found: 373.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 99.38%, Rt=8.912 min.

¹H NMR (400 MHz, DMSO-d₆) δ 1.70 (6H, s), 2.41 (3H, s), 3.24 (2H, t, J=6.8 Hz), 4.60 (2H, t, J=6.8 Hz), 7.41-7.47 (1H, m), 7.52-7.57 (2H, m), 8.01 (1H, m), 8.51 (1H, s).

The synthesis of 3-acetamido—S—Chloro-N-(2-(1-(2-mercaptoethyl)-1H-1,2,3-triazol-4-yl)propan-2-yl)benzamide (SU20666-166).

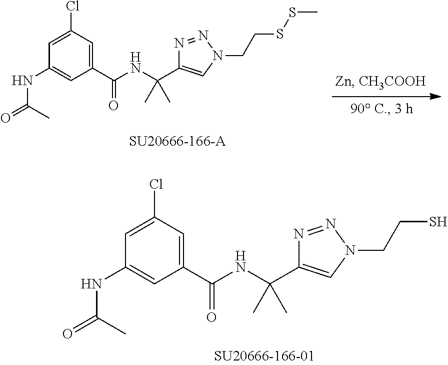 [SMILES: CSSCCn1cc(C(C)(C)NC(=O)c2cc(Cl)cc(NC(C)=O)c2)nn1 ; CC(=O)Nc1cc(Cl)cc(C(=O)NC(C)(C)c2cn(CCS)nn2)c1]

To a solution of compound 166-A (175.0 mg, 0.41 mmol) in Acetic acid glacial (5 mL) was added Zinc powder (81.0 mg, 1.23 mmol). The mixture was stirred at 90° C. for 1 h. After the consumption of starting material (by LCMS), the solvent was removed in vacuo, the crude was quenched with 20 mL water, and extracted with CH₂Cl₂ (20×3 mL), combined the organic layer and dried over anhydrous Na₂SO₄, filtered and concentrated in vacuo, the crude was purified by Prep-HPLC to give the product 166 (19.0 mg, yield: 10.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 97.47%, Rt=1.496 min; MS Calcd.: 381.0; MS Found: 382.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 97.41%, Rt=7.265 min.

¹H NMR (400 MHz, DMSO-d₆) δ 1.69 (6H, s), 2.05 (3H, s), 2.41 (1H, d, J=6.0 Hz), 2.92-2.96 (2H, m), 4.46 (2H, t, J=6.8 Hz), 7.56 (1H, s), 7.73 (1H, s), 7.96-7.99 (2H, m), 8.46 (1H, s), 10.25 (1H, s).

The names SU20666-168, SP 168, and 168 all refer to the same compound having the formula:

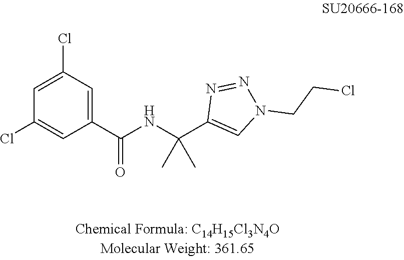 [SMILES: CC(C)(NC(=O)c1cc(Cl)cc(Cl)c1)c1cn(CCCl)nn1]

Route for SU20666-168

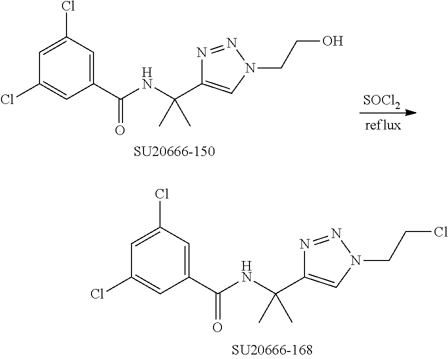

The synthesis of 3,5-dichloro-N-(2-(1-(2-chloroethyl)-1H-1,2,3-triazol-4-yl)propan-2-yl)benzamide (SU20666-168).

To a solution of compound SU20666-150 (50 mg, 0.146 mmol) in SOCl₂ (25 ml). The mixture was heated to reflux for 16 h. The mixture was concentrated under vacuum and purified by prep-HPLC to give SU20666-168 (18.54 mg, yield: 35.25%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.799 min; MS Calcd.: 360; MS Found: 361.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 98.37%, Rt=9.144 min; MS Calcd.: 360; MS Found: 361.0 [M+H]⁺.

¹H NMR (400 MHz, DMSO-d₆) δ 1.70 (6H, s), 4.06 (2H, t, J=6.0 Hz), 4.66 (2H, t, J=5.6Hz), 7.79 (1H, t, J=2.0 Hz) , 7.84 (2H, d, J=2.0 Hz), 8.01 (1H, s), 8.62 (1H, s).

The names SU20666-172, SP 172, and 172 all refer to the same compound having the formula:

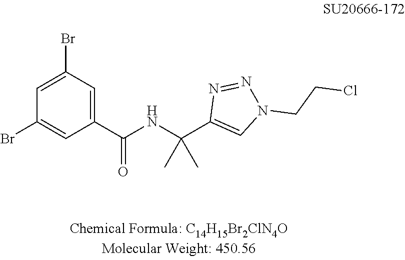 [SMILES: CC(C)(NC(=O)c1cc(Br)cc(Br)c1)c1cn(CCCl)nn1]

Route for SU20666-172

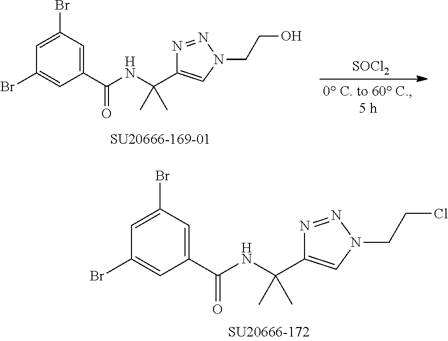

The synthesis of 3,5-dibromo-N-(2-(1-(2-chloroethyl)-1H-1,2,3-triazol-4-yl)propan-2-yl)benzamide (SU20666-172).

To a solution of compound 169 (107.0 mg, 0.25 mmol) in Thionyl chloride (5 mL) at 0° C., then The mixture was warmed to 60° C. for 5 h. After the consumption of starting material (by LCMS), the solvent was removed in vacuo, the crude was dissolved with 20 mL water, the aqueous phase was extracted with CH₂Cl₂ (20×3 mL), combined the organic layer and dried over anhydrous Na₂SO₄, filtered and concentrated in vacuo, the crude was dissolved with DMF and purified by Prep-HPLC to give the desired product 172 (37.0 mg, yield: 33.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% NH₄HCO₃] and 5% [CH₃CN+0.05% NH₄HCO₃] to 5% [water+0.05% NH₄HCO₃] and 95% [water+0.05% NH₄HCO₃] in 1.6 min, then under this condition for 1.4 min, finally changed to 95% [water+0.05% NH₄HCO₃] and 5% [CH₃CN+0.05% NH₄HCO₃] in 0.05 min and under this condition for 0.7 min), Purity: 95.32%, Rt=2.038 min; MS Calcd.: 448.0; MS Found: 449.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 94.47%, Rt=9.430 min.

¹H NMR (400 MHz, DMSO-d₆) δ 1.70 (6H, s), 4.05 (2H, t, J=5.6 Hz), 4.66 (2H, t, J=5.6), 8.00-8.01 (4H, m), 8.62 (1H, s).

The names SU20666-178, SP 178, and 178 all refer to the same compound having the formula:

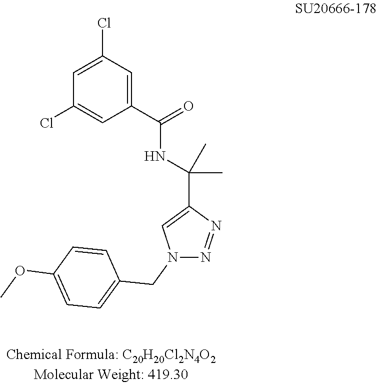 [SMILES: COc1ccc(Cn2cc(C(C)(C)NC(=O)c3cc(Cl)cc(Cl)c3)nn2)cc1]

Route for SU20666-178

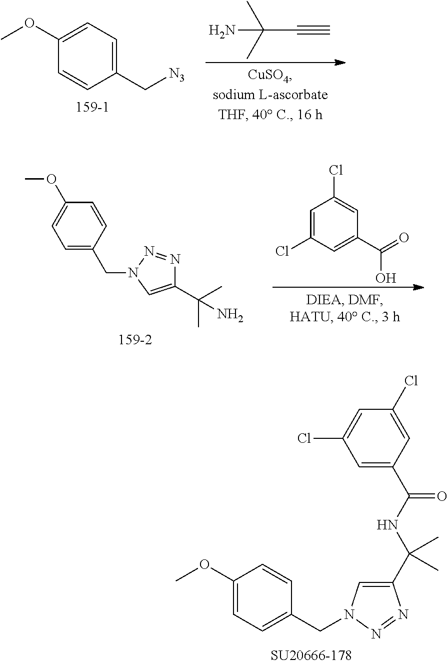

The synthesis of 2-(1-(4-methoxybenzyl)-1H-1,2,3-triazol-4-yl)propan-2-amine (159-2).

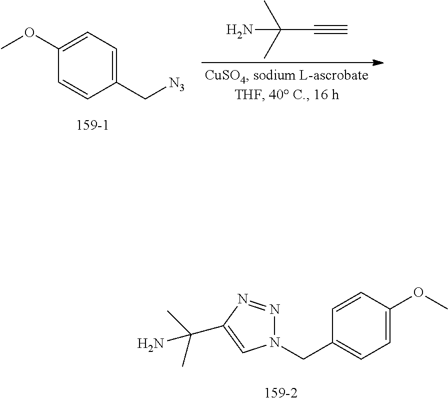

To a solution of compound 159-1 (2.5 g, 15.32 mmol) in THF (150 ml) was added copper sulfate pentahydrate (3.83 g, 15.32 mmol), 2-methylbut-3-yn-2-amine (1.28 g, 15.32 mmol) and sodium L-ascorbate(3.03 g, 15.32 mmol). The mixture was heated to 40° C. for 16 h. After the consumption of starting material (by LCMS), the mixture was concentrated under vacuum then water (20 mL) was added. The mixture was freeze-dried under vacuum to give 159-2 (10.94 g, crude) without further purification for the next step.

The synthesis of 3,5-dichloro-N-(2-(1-(4-methoxybenzyl)-1H-1,2,3-triazol-4-yl)propan-2-yl)benzamide (SU20666-178).

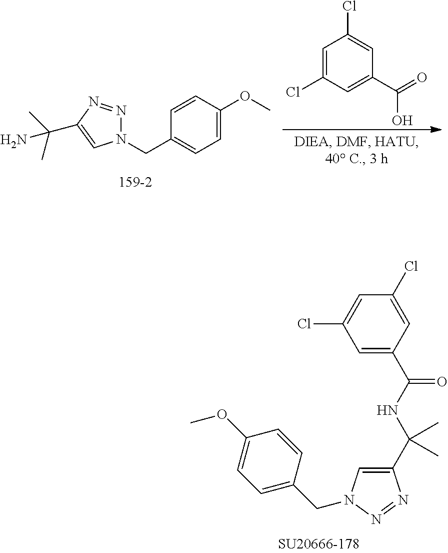

To a solution of compound 159-2 (10.94 g, 15.32 mmol) in DMF (250 mL) was added DIEA (2.58 g, 19.92 mmol), HATU (11.65 g, 30.64 mmol) and 3,5-dichlorobenzoic acid (2.93 g, 15.32 mmol). The mixture was stirred for 3 h at 40° C. After the consumption of starting material (by LCMS), water was added, the mixture was extracted by DCM. The combined organic phase was concentrated under vacuum and purified by prep-HPLC to give SU20666-178 (3.5 g, yield: 53.85%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100%, Rt=1.913 min; MS Calcd.: 418; MS Found: 419.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 100%, Rt=9.826 min; MS Calcd.: 418; MS Found: 419.0 [M+H]⁺.

¹H NMR (400 MHz, DMSO-d₆) δ 1.67 (6H, s), 3.73 (3H, s), 5.44 (2H, s), 6.92 (2H, d, J=8.8 Hz), 7.28 (2H, d, J=8.8 Hz), 7.78 (1H, t, J=1.6 Hz), 7.82 (2H, d, J=2.0 Hz), 7.96 (1H, s), 8.59 (1H, s).

The names SU20666-179, SP 179, and 179 all refer to the same compound having the formula:

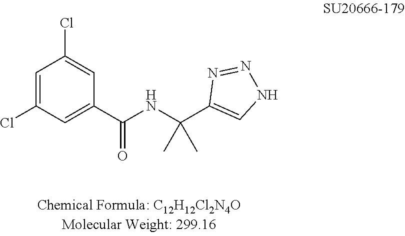 [SMILES: CC(C)(NC(=O)c1cc(Cl)cc(Cl)c1)c1c[nH]nn1]

Route for SU20666-179

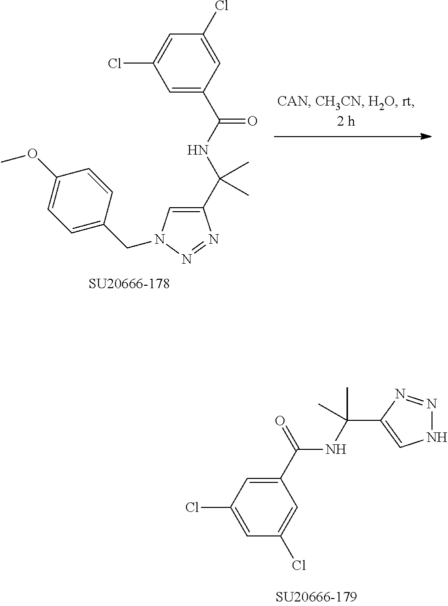 [SMILES: COc1ccc(Cn2cc(C(C)(C)NC(=O)c3cc(Cl)cc(Cl)c3)nn2)cc1 ; CC(C)(NC(=O)c1cc(Cl)cc(Cl)c1)c1c[nH]nn1]

The synthesis of 3,5-dichloro-N-(2-(1-(4-methoxybenzyl)-1H-1,2,3-triazol-4-yl)propan-2-yl)benzamide (SU20666-179).

To a solution of compound SU20666-178-01 (3.0 g, 7.15 mmol) in acetonitrile (300 ml) and H₂O (100 ml) was added CAN (43.12 g, 7.86 mmol). The mixture was stirred for 2 h at rt. The mixture was concentrated under vacuum and purified by prep-HPLC to give SU20666-179 (1.83 g, yield: 85.5%) as a yellow solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.25%, Rt=1.716 min; MS Calcd.: 298; MS Found: 299.0 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 95.26%, Rt=8.312 min; MS Calcd.: 298; MS Found: 299.0 [M+H]⁺.

¹H NMR (400 MHz, DMSO-d₆) δ 1.69 (6H, s), 7.58-7.68 (1H, m), 7.79 (1H, t, J=2.0 Hz), 7.84 (2H, d, J=2.0 Hz) , 8.62 (1H, s).

The names SU20666-180, SP 180, and 180 all refer to the same compound having the formula:

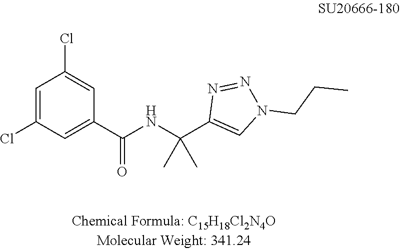 [SMILES: CCCn1cc(C(C)(C)NC(=O)c2cc(Cl)cc(Cl)c2)nn1]

Route for SU20666-168

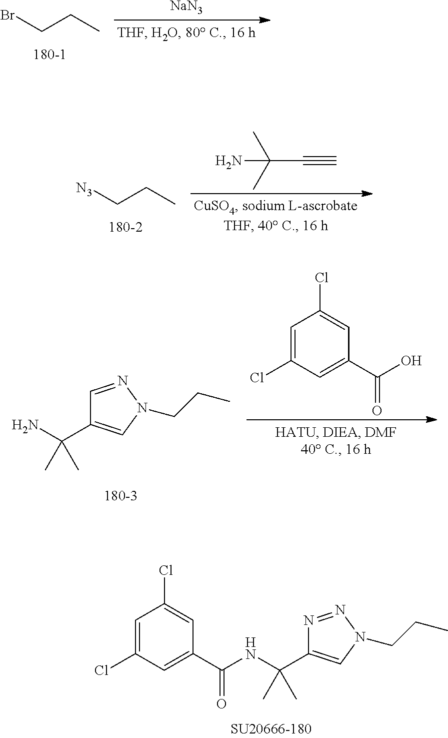

The synthesis of 1-azidopropane (180-2).

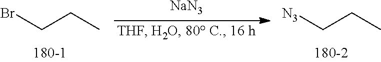

To a solution of compound 150-1 (200 mg, 1.63 mmol) in THF (10 ml) and H₂O (10 ml) was added NaN₃ (126.84 mg, 1.95 mmol). The mixture was heated to 80° C. for 16 h. The mixture was concentrated under vacuum and extracted with DCM three tiomes. The combined organic phase was concentrated under vacuum to give 180-2 (100 mg, yield: 72%) as a white solid.

The synthesis of 2-(1-nronvl-1H-pyrazol-4-yl)pronan-2-amine (180-3).

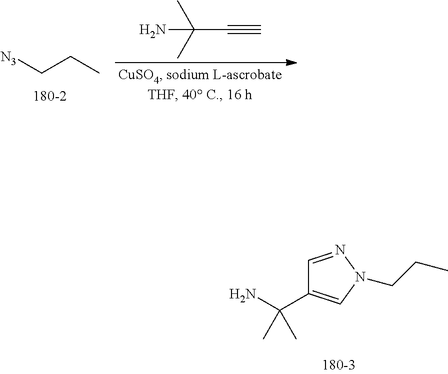

To a solution of compound 180-2 (100 mg, 1.18 mmol) in THF (50 ml) was added Copper sulfate pentahydrate (22.7 mg, 1.29 mmol), 2-methylbut-3-yn-2-amine (107.5 mg, 1.29 mmol) and sodium L-ascorbate(256 mg, 1.29 mmol). The mixture was heated to 40° C. for 16 h. After the consumption of starting material (by LCMS), the mixture was concentrated under vacuum, and water (20 mL) was added. The mixture was freeze-dried under vacuum to give 180-3 (450 mg, crude) without further purification for the next step.

The synthesis of 3,5-dichloro-N-(2-(1-propyl-1H-1,2,3-triazol-4-yl)propan-2-yl)benzamide (SU20666-180).

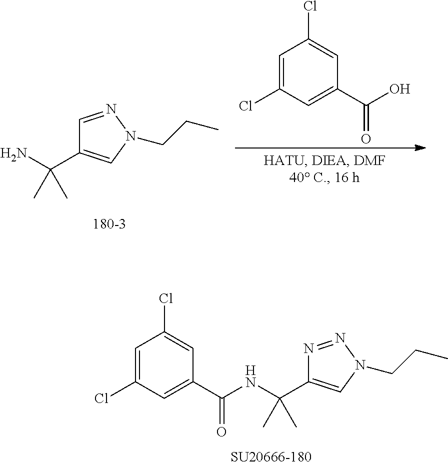

To a solution of compound 180-3 (450 mg (crude), 1.18 mmol) in DMF (25 mL) was added DIEA (305 mg, 2.36 mmol), HATU (538.4 mg, 1.42 mmol) and 3,5-dichlorobenzoic acid (225.4 mg, 1.178 mmol). The mixture was stirred for 16 h at 40° C. After the consumption of starting material (by LCMS). Then added water, the aqueous phase was extracted with dichloromethane, the combined organic phases were dried over anhydrous sodium sulfate, filtered, and concentrated in vacuopurified by prep-HPLC to give the desired product SU20666-180 (20 mg, yield: 5%) as a white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 99.67%, Rt=2.138 min; MS Calcd.: 340; MS Found: 341.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min), Purity: 98.96%, Rt=9.320 min; MS Calcd.: 340; MS Found: 341.2 [M+H]⁺.

¹H NMR (400 MHz, DMSO-d₆) δ 0.83 (3H, t, J=7.6 Hz), 1.69 (6H, s), 1.76-1.85 (2H, m), 4.25 (2H, t, J=6.4 Hz), 7.78 (1H, t, J=2.0 Hz), 7.84 (2H, d, J=1.6 Hz), 7.93 (1H, s), 8.59 (1H, s).

The names SU20666-181, SP 181, and 181 all refer to the same compound having the formula:

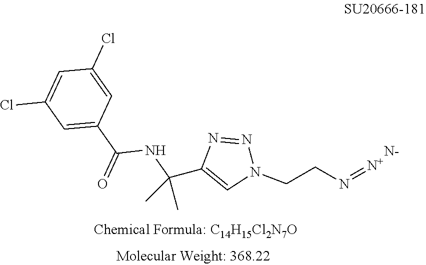

Route for SU20666-181

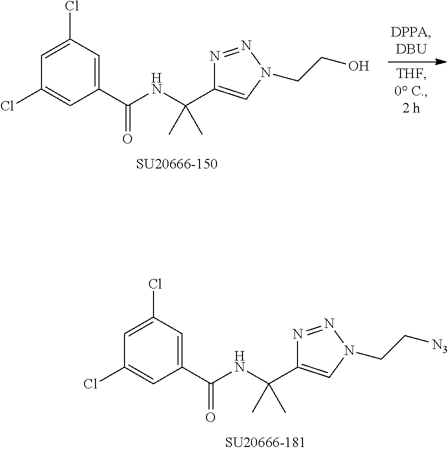 [SMILES: CC(C)(NC(=O)c1cc(Cl)cc(Cl)c1)c1cn(CCO)nn1 ; CC(C)(NC(=O)c1cc(Cl)cc(Cl)c1)c1cn(CCN=[N+]=[N-])nn1]

The synthesis of N-(2-(1-(2-azidoethyl)-1H-1,2,3-triazol-4-yl)propan-2-yl)-3,5-dichlorobenzamide (SU20666-181).

To a solution of compound 150-01 (1.0 g, 2.91 mmol) in THF (30 ml) was added DPPA (1.04 g, 3.79 mmol) and DBU (577 mg, 3.79 mmol). The mixture was stirred at 0° C. for 2 h. After the consumption of starting material (by LCMS), then added water, the aqueous phase was extracted with dichloromethane, the combined organic phases were dried over anhydrous sodium sulfate, filtered, and concentrated in vacuopurified by prep-HPLC to give the desired product SU20666-181 (600 mg, yield: 56%) as a white solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 100%, Rt=1.935 min; MS Calcd.: 367; MS Found: 368.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 100%, Rt=9.256 min; MS Calcd.: 367; MS Found: 368.2 [M+H]⁺.

¹H NMR (400 MHz, DMSO-d₆) δ 1.70 (6H, s), 3.80 (2H, t, J=6.0 Hz), 4.5 (2H, t, J=5.6 Hz), 7.78 (1H, t, J=2.0 Hz), 7.84 (2H, d, J=2.0 Hz), 8.00 (1H, s), 8.62 (1H, s)

The names SU20666-186, SP 186, and 186 all refer to the same compound having the formula:

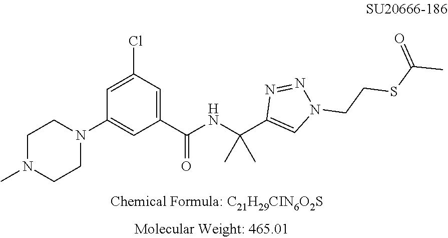 [SMILES: CC(=O)SCCn1cc(C(C)(C)NC(=O)c2cc(Cl)cc(N3CCN(C)CC3)c2)nn1]

Route for SU20666-186

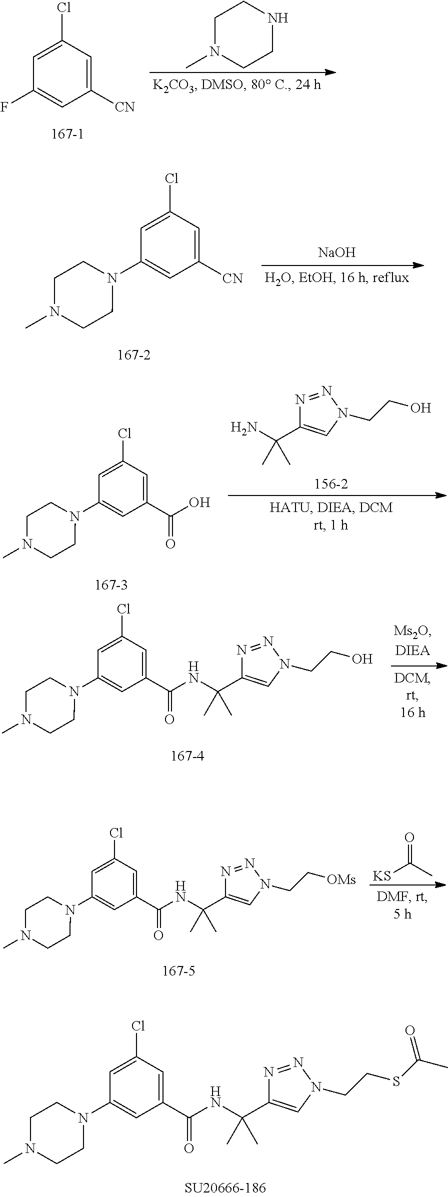

The synthesis of 3-chloro—S—(4-methylpiperazin-1-yl)benzonitrile (167-2).

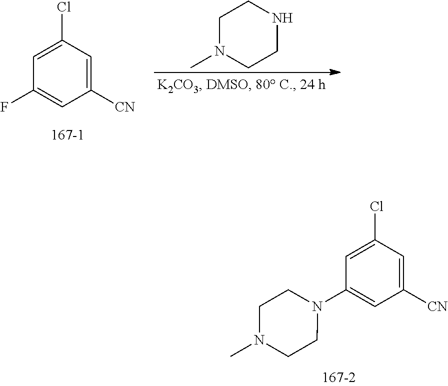

To a solution of compound 167-1 (623.0 mg, 4.0 mmol) in DMSO (10 mL) was added K₂CO₃ (1.1 g, 8.0 mmol) and 1-methylpiperazine (601.0 mg, 6.0 mmol). The mixture was warmed to 80° C. for 24 h. After the consumption of starting material (by LCMS), the reaction solvent was quenched with 100 mL water, and extracted with EtOAc (30×3 mL), the organic layer was washed with saturated aqueous NaCl, collected the organic layer and dried over anhydrous Na₂SO₄, filtered and concentrated in vacuo, the crude was used derectly for next step without further purification, the crude 167-2 (910.0 mg, yield: 96.8%) was get as a white solid.

The synthesis of 3-chloro—S—(4-methylpiperazin-1-yl)benzoic acid (167-3).

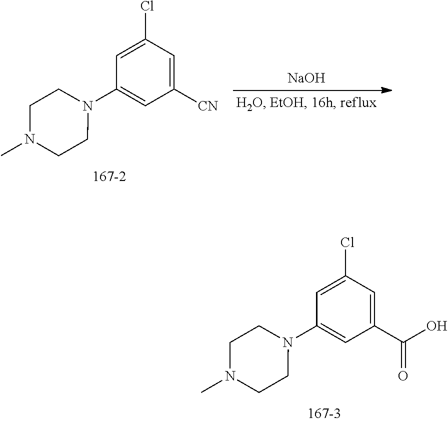 [SMILES: CN1CCN(c2cc(Cl)cc(C#N)c2)CC1 ; "NaOH" ; CN1CCN(c2cc(Cl)cc(C(=O)O)c2)CC1]

To a solution of compound 167-2 (910.0 mg, 3.9 mmol) in 95% EtOH (15 mL) was added NaOH (165.0 mg, 11.6 mmol). The mixture was refluxed for 16 h. After the consumption of starting material (by LCMS), the solvent was removed in vacuo, the crude was dissolved with 30 mL water, and 1N aqueous HCl was added to adjust PH=2-3, the water phase was extracted with CH₂Cl₂:CH₃OH=10:1 (30×5 mL), combined the organic layer and dried over anhydrous Na₂SO₄, filtered and concentrated in vacuo, the crude was used directly for next step without further purification, the crude 167-3 (800.0 mg, yield: 81.3%) was obtained as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 0% [water+0.05% TFA] and 100% [CH₃CN+0.05% TFA] to 95% [water+0.05% TFA] and 5% [CH₃CN+0.05% TFA] in 0.01 min, Purity: 100.00%, Rt=0.443 min; MS Calcd.: 254.0; MS Found: 255.2 [M+H]⁺.

The synthesis of 3-chloro-N-(2-(1-(2-hydroxyethyl)-1H-1,2,3-triazol-4-yl)propan-2-yl)-5-(4-methylpiperazin-1-yl)benzamide (167-4).

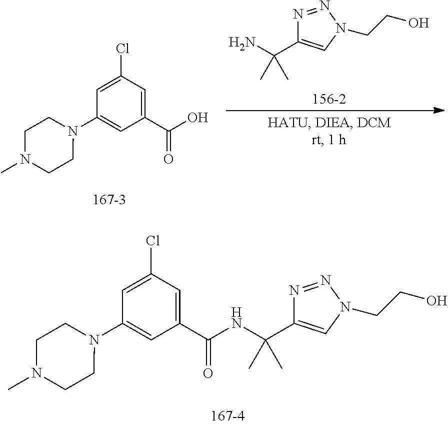 [SMILES: CN1CCN(c2cc(Cl)cc(C(=O)O)c2)CC1 ; CC(C)(N)c1cn(CCO)nn1 ; CN1CCN(c2cc(Cl)cc(C(=O)NC(C)(C)c3cn(CCO)nn3)c2)CC1]

To a solution of compound 167-3 (508.0 mg, 2.0 mmol) in CH₂Cl₂ (20 mL) was added HATU (1.2 g, 6.0 mmol), DIEA (1.3 mL, 8.0 mmol) and 156-2 (340.0 mg, 2.0 mmol). The mixture was stirred at rt for 1 h. After the consumption of starting material (by LCMS), the mixture was quenched with 30 mL water, and extracted with CH₂Cl₂:CH₃OH (30×4 mL), combined the organic layer and dried over anhydrous Na₂SO₄, filtered and concentrated in vacuo, the crude was purified by flash-chromatography to give the desired product 167-4 (500.0 mg, yield: 61.6%) as a white solid.

The synthesis of 2-(4-(2-(3-chloro-5-(4-methylpiperazin-1-yl)benzamido)propan-2-yl)-1H-1,2,3-triazol-1-yl)ethyl 4-methylbenzenesulfonate (167-5).

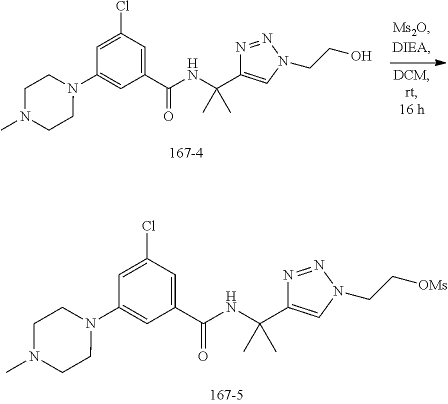 [SMILES: CN1CCN(c2cc(Cl)cc(C(=O)NC(C)(C)c3cn(CCO)nn3)c2)CC1 ; CN1CCN(c2cc(Cl)cc(C(=O)NC(C)(C)c3cn(CCOS(C)(=O)=O)nn3)c2)CC1]

To a solution of compound 167-4 (812.0 mg, 2.0 mmol) in CH₂Cl₂ (20 mL) was added DIEA (517.0 mg, 4.0 mmol) and Ms₂O (523.0 mg, 3.0 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 167-5 (850.0 mg, yield: 87.8%) as a white solid.

The synthesis of S-2-(4-(2-(3-chloro-5-(4-methylpiperazin-1-yl)benzamido)propan-2-yl)-1H-1,2,3-triazol-1-yl)ethyl ethanethioate (SU20666-186).

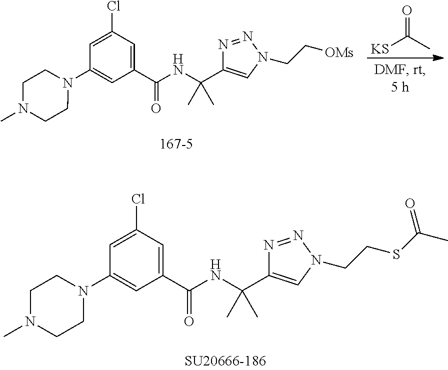

To a solution of compound 167-5 (968.0 mg, 2.0 mmol) in DMF (5 mL) was added Potassium thioacetate (343.0 mg, 3.0 mmol). The mixture was stirred at rt for 5 h. After the consumption of starting material (by LCMS), the mixture was quenched with 50 mL water, and extracted with EtOAc (30×4 mL), combined the organic layer and dried over anhydrous Na₂SO₄, filtered and concentrated in vacuo, the crude was purified by Prep-HPLC and Prep-TLC to give the desired product 186 (510.0 mg, yield: 55.0%) as a yellow solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 97.62%, Rt=1.714 min; MS Calcd.: 464.0; MS Found: 465.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 96.62%, Rt=8.258 min.

¹H NMR (400 MHz, CDCl₃) δ 1.80 (6H, s), 2.31 (6H, d, J=2.4 Hz), 2.52 (4H, t, J=4.4 Hz), 3.21 (4H, t, J=4.8 Hz), 3.30 (2H, t, J=6.8 Hz), 4.44 (2H, t, J=6.8 Hz), 6.78 (1H, s), 6.88 (1H, t, J=2.0 Hz), 7.01 (1H, t, J=1.2 Hz), 7.15 (1H, t, J=2.0 Hz), 7.53 (1H, s).

The names SU20666-191, SP 191, and 191 all refer to the same compound having the formula as shown below. The names SU20666-193, SP 193, and 193 all refer to the same compound having the formula as shown below.

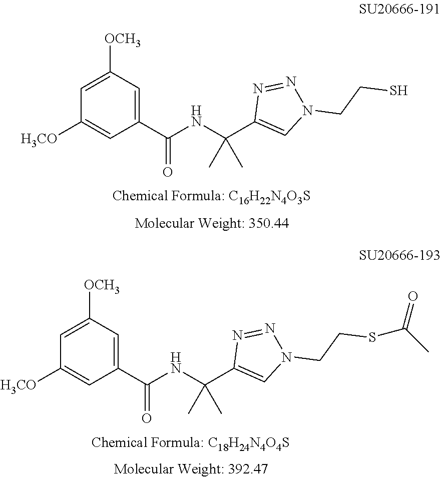 [SMILES: COc1cc(OC)cc(C(=O)NC(C)(C)c2cn(CCS)nn2)c1 ; COc1cc(OC)cc(C(=O)NC(C)(C)c2cn(CCSC(C)=O)nn2)c1]

Route for SU20666-191 and SU20666-193

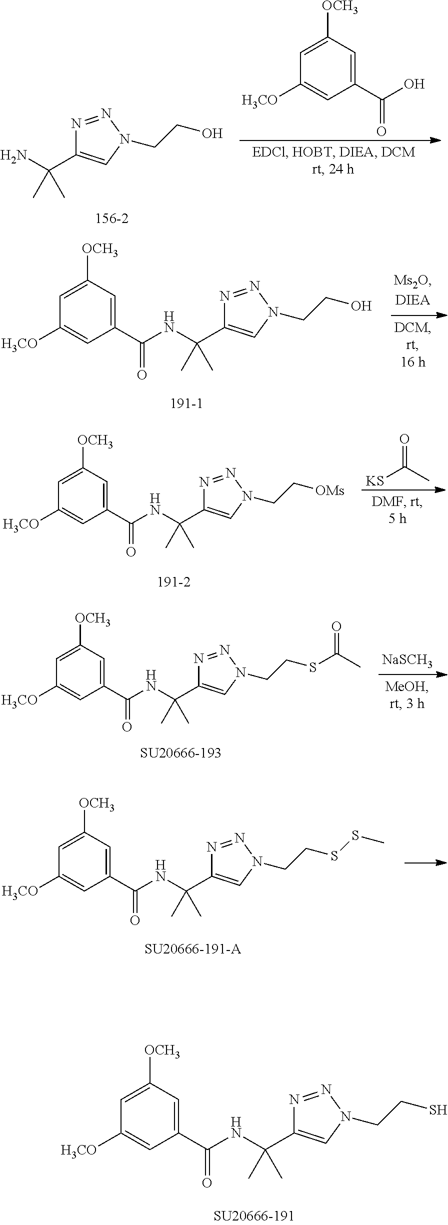

The synthesis of N-(2-(1-(2-hydroxyethyl)-1H-1,2,3-triazol-4-yl)propan-2-yl)-3,5-dimethoxybenzamide (191-1).

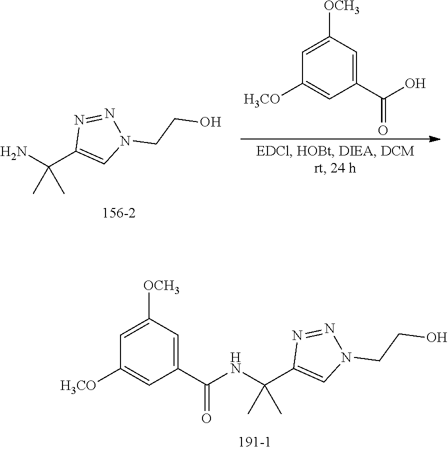 [SMILES: CC(C)(N)c1cn(CCO)nn1 ; COc1cc(OC)cc(C(=O)O)c1 ; COc1cc(OC)cc(C(=O)NC(C)(C)c2cn(CCO)nn2)c1]

To a solution of compound 3,5-dimethoxybenzoic acid (365.0 mg, 2.0 mmol) in CH₂Cl₂ (25 mL) was added EDCI (460.0 mg, 2.4 mmol), HOBT (325.0 mg, 2.4 mmol), DIEA (517.0 mg, 4.0 mmol), the mixture was stirred at rt for 10 min, then, 156-2 (340.0 mg, 2.0 mmol) was dissolved in 5 mL CH₂Cl₂ to added into the reaction mixture. The mixture was stirred at rt for 24 h. After the consumption of starting material (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 191-1 (453.0 mg, yield: 67.8%) as a white solid.

The synthesis of 2-(4-(2-(3,5-dimethoxybenzamido)propan-2-yl)-1H-1,2,3-triazol-1-yl)ethyl methanesulfonate (191-2).

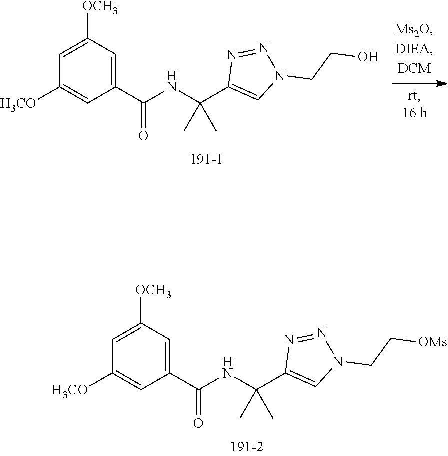 [SMILES: COc1cc(OC)cc(C(=O)NC(C)(C)c2cn(CCO)nn2)c1 ; COc1cc(OC)cc(C(=O)NC(C)(C)c2cn(CCOS(C)(=O)=O)nn2)c1]

To a solution of compound 191-1 (400.0 mg, 1.2 mmol) in CH₂Cl₂ (10 mL) was added DIEA (310.0 mg, 2.4 mmol) and Ms₂O (314.0 mg, 1.8 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 191-2 (480.0 mg, yield: 97.0%) as a white solid.

The synthesis of S-2-(4-(2-(3,5-dimethoxybenzamido)propan-2-yl)-1H-1,2,3-triazol-1-yl)ethyl ethanethioate (SU20666-193).

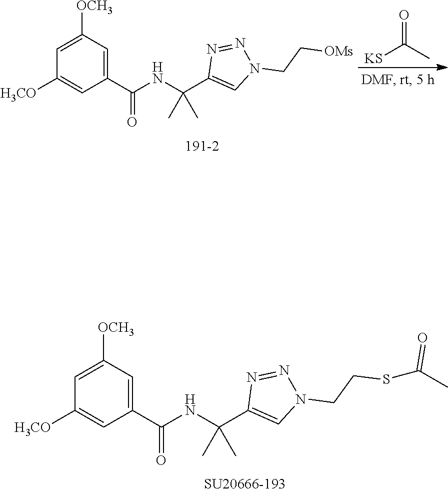

To a solution of compound 191-2 (555.0 mg, 1.4 mmol) in DMF (5 mL) was added Potassium thioacetate (231.0 mg, 2.0 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by Prep-HPLC to give the desired product 193 (398.0 mg, yield: 75.0%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 93.48%, Rt=1.625 min; MS Calcd.: 392.0; MS Found: 393.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 95.44%, Rt=7.486 min.

¹H NMR (400 MHz, CDCl₃) δ 1.81 (6H, s), 3.29 (2H, t, J=6.8 Hz), 3.74 (6H, s), 4.43 (2H, t, J=6.8 Hz), 6.49 (1H, t, J=2.4 Hz), 6.81 (3H, t, J=2.8 Hz), 7.54 (1H, s).

The synthesis of 3,5-dimethoxy-N-(2-(1-(2-(methyldisulfanyl)ethyl)-1H-1,2,3-triazol-4-yl)propan-2-yl)benzamide (SU20666-191-A).

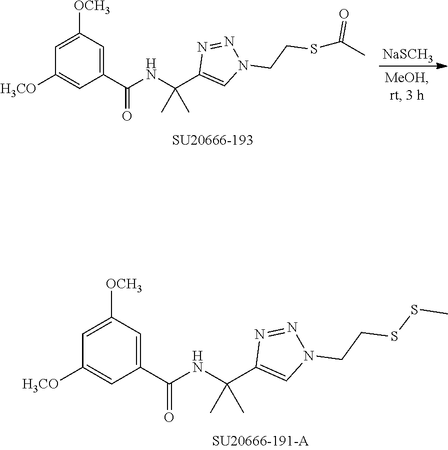

To a solution of compound 193 (286.0 mg, 0.73 mmol) in CH₃OH (10 mL) was added Sodium thiomethoxide (77.0 mg, 1.09 mmol). The mixture was stirred at rt for 3 h. After the consumption of starting material (by LCMS), the mixture was quenched with 20 mL water, and extracted with CH₂Cl₂ (20×3 mL), combined the organic layer and dried over anhydrous Na₂SO₄, filtered and concentrated in vacuo to give the product 191-A (150.0 mg, yield: 51.9%) as a white solid.

The synthesis of N-(2-(1-(2-mercaptoethyl)-1H-1,2,3-triazol-4-yl)propan-2-yl)-3,5-dimethoxybenzamide (SU20666-191).

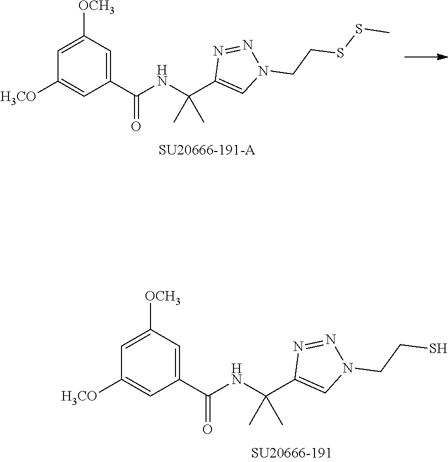 [SMILES: COc1cc(OC)cc(C(=O)NC(C)(C)c2cn(CCSSC)nn2)c1 ; COc1cc(OC)cc(C(=O)NC(C)(C)c2cn(CCS)nn2)c1]

To a solution of compound 191-A (150.0 mg, 0.38 mmol) in Acetic acid glacial (5 mL) was added Zinc powder (75.0 mg, 1.14 mmol). The mixture was stirred at 90° C. for 1 h. After the consumption of starting material (by LCMS), the solvent was remove in vacuo, the crude was quenched with 20 mL water, and extracted with CH₂Cl₂ (20×3 mL), combined the organic layer and dried over anhydrous Na₂SO₄, filtered and concentrated in vacuo, the crude was purified by Prep-HPLC to give the product 191 (33.0 mg, yield: 24.8%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 98.74%, Rt=1.579 min; MS Calcd.: 350.0; MS Found: 351.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 95.08%, Rt=7.265 min.

¹H NMR (400 MHz, DMSO-d₆) δ 1.70 (6H, s), 2.50 (1H, t, J=1.6 Hz), 2.95 (2H, t, J=6.8 Hz), 3.78 (6H, d, J=3.2 Hz), 4.46 (2H, t, J=6.8 Hz), 6.63 (1H, t, J=2.0 Hz), 6.96 (2H, d, J=2.4 Hz), 7.94 (1H, s), 8.29 (1H, s).

The names SU20666-194, SP 194, and 194 all refer to the same compound having the formula:

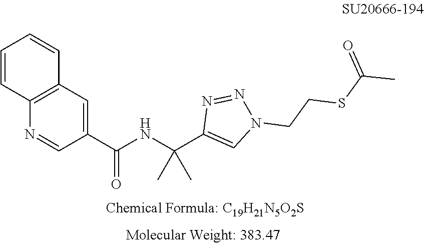 [SMILES: CC(=O)SCCn1cc(C(C)(C)NC(=O)c2cnc3ccccc3c2)nn1]

Route for SU20666-194

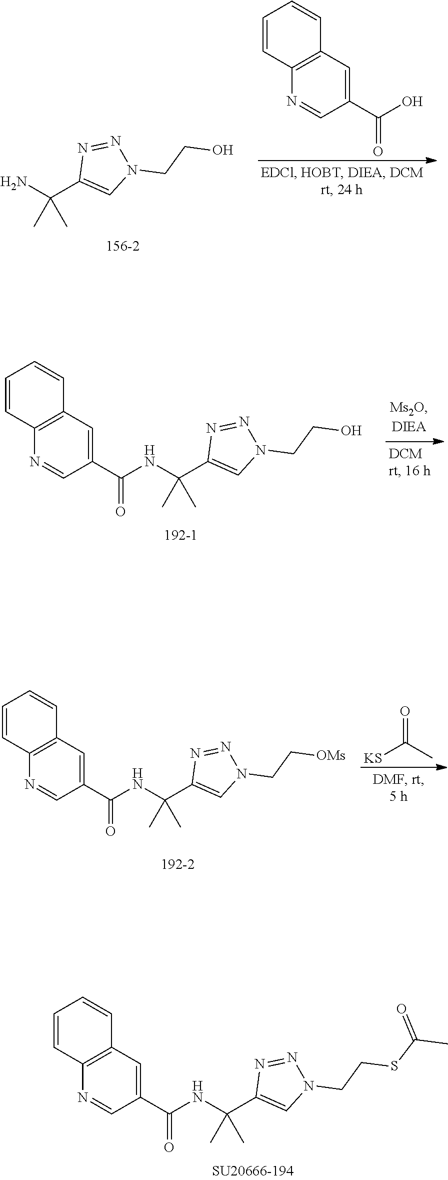

The synthesis of N-(2-(1-(2-hydroxyethyl)-1H-1,2,3-triazol-4-yl)propan-2-yl)quinoline-3-carboxamide (192-1).

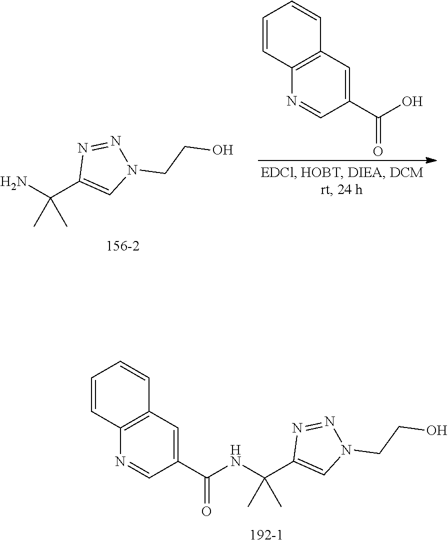 [SMILES: CC(C)(N)c1cn(CCO)nn1 ; O=C(O)c1cnc2ccccc2c1 ; CC(C)(NC(=O)c1cnc2ccccc2c1)c1cn(CCO)nn1]

To a solution of compound quinoline-3-carboxylic acid (346.0 mg, 2.0 mmol) in CH₂Cl₂ (25 mL) was added EDCI (460.0 mg, 2.4 mmol), HOBT (325.0 mg, 2.4 mmol), DIEA (517.0 mg, 4.0 mmol), the mixture was stirred at rt for 10 min, then, 156-2 (340.0 mg, 2.0 mmol) was dissolved in 5 mL CH₂Cl₂ to added into the reaction mixture. The mixture was stirred at rt for 24 h. After the consumption of starting material (by LCMS), the mixture was quenched with 30 mL water, and extracted with CH₂Cl₂:CH₃OH (30×5 mL), combined the organic layer and dried over anhydrous Na₂SO₄, filtered and concentrated in vacuo, the crude was purified by flash-chromatography to give the desired product 192-1 (446.0 mg, yield: 68.6%) as a white solid.

The synthesis of 2-(4-(2-(quinoline-3-carboxamido)propan-2-yl)-1H-1,2,3-triazol-1-yl)ethyl methanesulfonate (192-2).

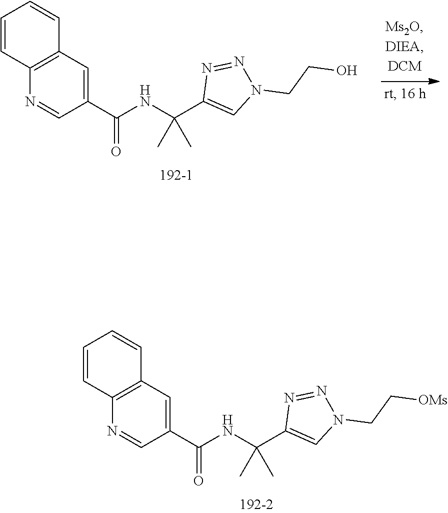 [SMILES: CC(C)(NC(=O)c1cnc2ccccc2c1)c1cn(CCO)nn1 ; CC(C)(NC(=O)c1cnc2ccccc2c1)c1cn(CCOS(C)(=O)=O)nn1]

To a solution of compound 192-1 (390.0 mg, 1.2 mmol) in CH₂Cl₂ (10 mL) was added DIEA (310.0 mg, 2.4 mmol) and Ms₂O (314.0 mg, 1.8 mmol). The mixture was stirred at rt for 16 h. After the reaction was finished (by LCMS), the mixture was quenched with 30 mL water, and extracted with CH₂Cl₂ (30×3 mL), combined the organic layer and dried over anhydrous Na₂SO₄, filtered and concentrated in vacuo, the crude was purified by flash-chromatography to give the desired product 192-2 (475.0 mg, yield: 98.1%) as a white solid.

The synthesis of S-2-(4-(2-(quinoline-3-carboxamido)propan-2-yl)-1H-1,2,3-triazol-1-yl)ethyl ethanethioate (SU20666-194).

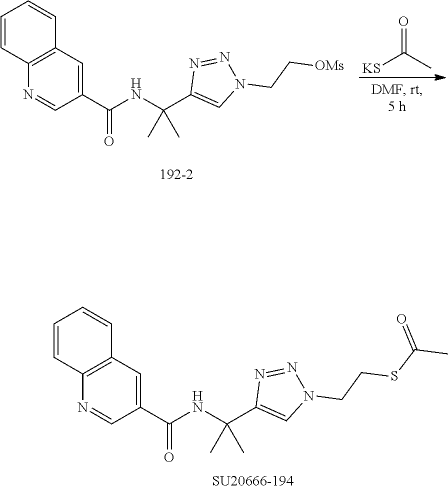

To a solution of compound 192-2 (490.0 mg, 1.2 mmol) in DMF (5 mL) was added Potassium thioacetate (209.0 mg, 1.8 mmol). The mixture was stirred at rt for 5 h. After the consumption of starting material (by LCMS), the mixture was quenched with 50 mL water, and extracted with EtOAc (30×3 mL), combined the organic layer and dried over anhydrous Na₂SO₄, filtered and concentrated in vacuo, the crude was purified by recrystallization petroleum ether/ethyl acetate to give the desired product 194 (350.0 mg, yield: 76.1%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=1.387 min; MS Calcd.: 383.0; MS Found: 384.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 100.00%, Rt=5.764 min.

¹H NMR (400 MHz, CDCl₃) δ 1.88 (6H, s), 2.32 (3H, s), 3.32 (2H, t, J=6.8 Hz), 4.47 (2H, t, J=6.8 Hz), 7.17 (1H, s), 7.53-7.58 (2H, m), 7.72-7.76 (1H, s), 7.84 (1H, d, J=8.0 Hz), 8.09 (1H, d, J=8.4 Hz), 8.49 (1H, d, J=2.0 Hz), 9.23 (1H, d, J=2.0 Hz).

The names SU20666-195, SP 195, and 195 all refer to the same compound having

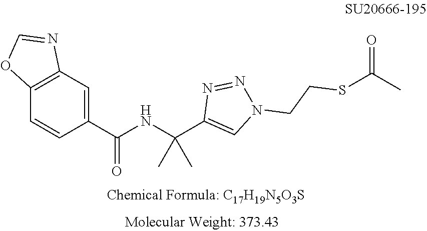 [SMILES: CC(=O)SCCn1cc(C(C)(C)NC(=O)c2ccc3ocnc3c2)nn1]

Route for SU20666-195

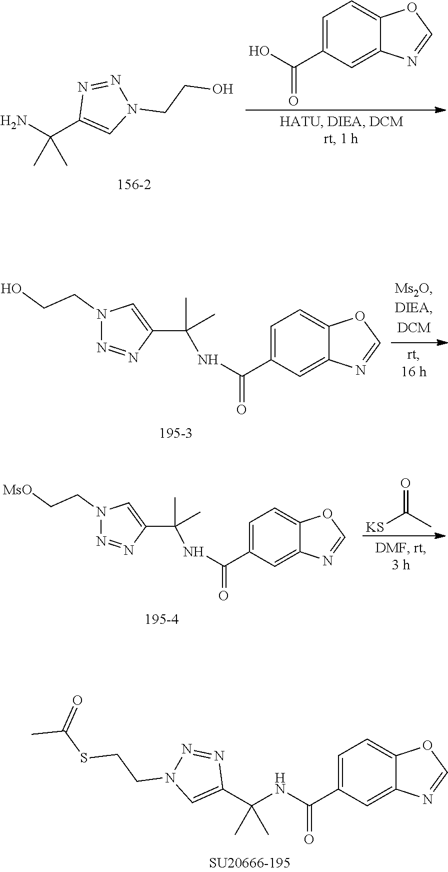

The synthesis of N-(2-(1-(2-hydroxyethyl)-1H-1,2,3-triazol-4-yl)propan-2-yl)benzo[d]oxazole—S—Carboxamide (195-3).

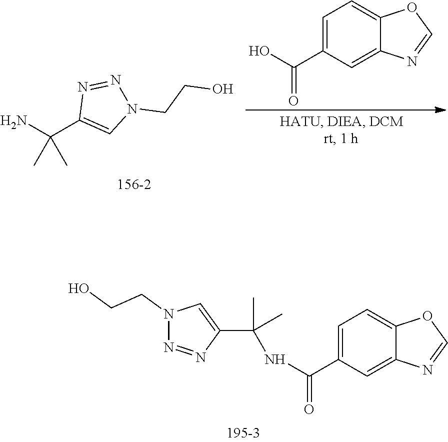 [SMILES: CC(C)(N)c1cn(CCO)nn1 ; O=C(O)c1ccc2ocnc2c1 ; CC(C)(NC(=O)c1ccc2ocnc2c1)c1cn(CCO)nn1]

To a solution of compound 156-2 (200 mg, 1.18 mmol) in DCM (15 mL) was added DIEA (303.72 mg, 2.36 mmol), HATU (583.28 mg, 1.54 mmol) and benzo[d]oxazole-5-carboxylic acid (192.5 mg, 1.18 mmol). The mixture was stirred for 1 h at rt. After the consumption of starting material (by LCMS), then added water, the aqueous phase was extracted with dichloromethane, the combined organic phases were dried over anhydrous sodium sulfate, filtered, and concentrated in vacuopurified by prep-TLC to give the desired product 195-3 (250 mg, yield: 67%) as a brown solid.

The synthesis of 2-(4-(2-(benzo[d]oxazole-5-carboxamido)propan-2-yl)-1H-1,2,3-triazol-1-yl)ethyl methanesulfonate (195-4).

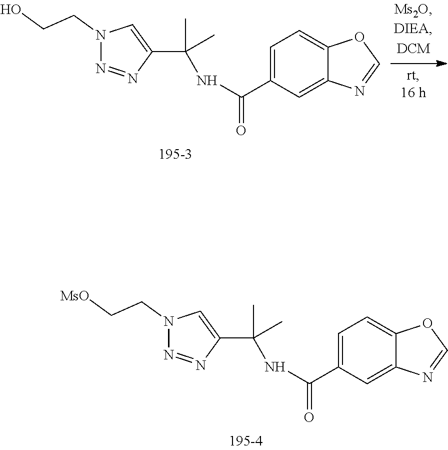 [SMILES: CC(C)(NC(=O)c1ccc2ocnc2c1)c1cn(CCO)nn1 ; CC(C)(NC(=O)c1ccc2ocnc2c1)c1cn(CCOS(C)(=O)=O)nn1]

To a solution of compound 195-3 (250 mg, 0.793 mmol) in DCM (15 mL) was added DIEA (303.72 mg, 2.36 mmol) and Ms₂O (179.58 mg, 1.03 mmol). The mixture was stirred for 16 h at rt. After the consumption of starting material (by LCMS), then added water, the aqueous phase was extracted with dichloromethane, the combined organic phases were dried over anhydrous sodium sulfate, filtered, and concentrated in vacuopurified by prep-TLC to give the desired product 195-4 (224 mg, yield: 67%) as a brown solid.

The synthesis of S-2-(4-(2-(benzo[d]oxazole-5-carboxamido)propan-2-yl)-1H-1,2,3-triazol-1-yl)ethyl ethanethioate (SU20666-195)

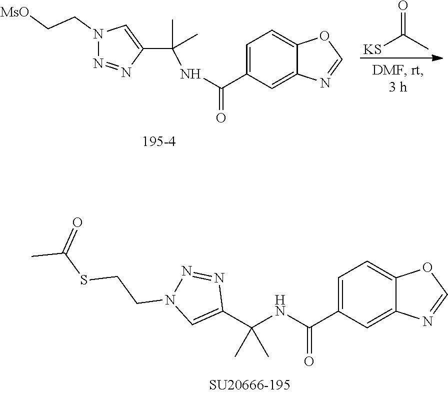

To a solution of compound 195-4 (224 mg, 0.57 mmol) in DMF (15 mL) was added Potassium thioacetate (65.03 mg, 0.57 mmol). The mixture was stirred for 3 h at rt. after the consumption of starting material (by LCMS), the mixture was purified by prep-TLC to give SU20666-195 (50 mg, yield: 23.5%) as a brown solid.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 95.21%, Rt=1.432 min; MS Calcd.: 373; MS Found:374.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 96.35%, Rt=7.081 min; MS Calcd.: 373; MS Found: 374.2 [M+H]⁺.

¹H NMR (400 MHz, DMSO-d₆) δ 1.73 (6H, s), 2.34 (3H, s), 3.34 (2H, t, J=6.8 Hz), 4.48 (2H, t, J=6.8 Hz), 7.83 (1H, d, J=8.8 Hz), 7.93 (1H, dd, J=8.4, 1.6 Hz), 7.97 (1H, s), 8.33 (1H, d, J=1.6 Hz), 8.45 (1H, s), 8.85 (1H, s).

The names SU20666-196, SP 196, and 196 all refer to the same compound having the formula:

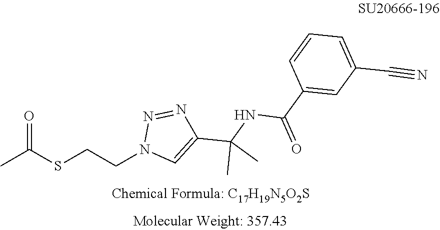 [SMILES: CC(=O)SCCn1cc(C(C)(C)NC(=O)c2cccc(C#N)c2)nn1]

Route for SU20666-196

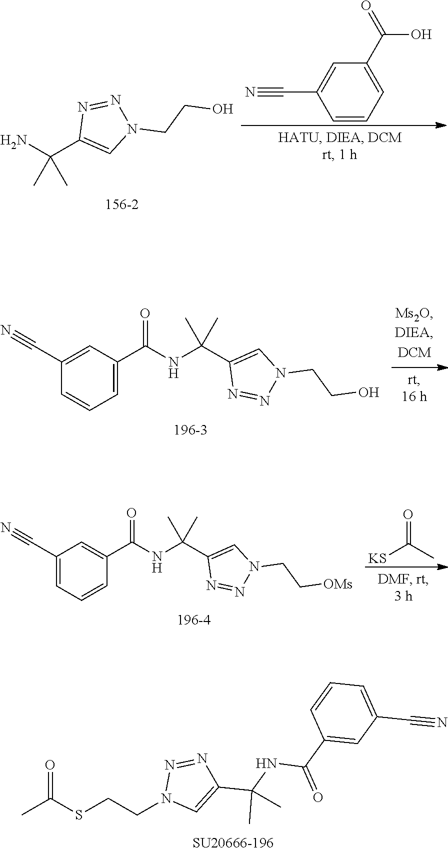

The synthesis of 3-cyano-N-(2-(1-(2-hydroxyethyl)-1H-1,2,3-triazol-4-yl)propan-2-yl)benzamide (196-3).

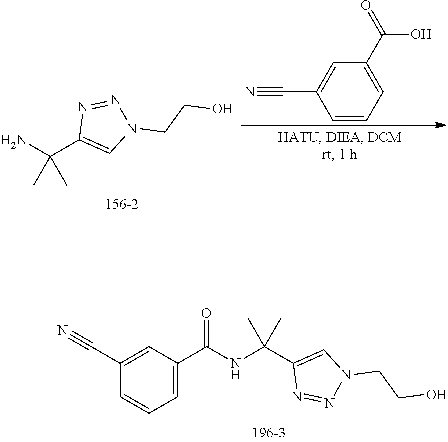

To a solution of compound 156-2 (200 mg, 1.18 mmol) in DCM (15 mL) was added DIEA (303.72 mg, 2.36 mmol), HATU (583.28 mg, 1.54 mmol) and 3-cyanobenzoic acid (173.7 mg, 1.18 mmol). The mixture was stirred for 1 h at rt. After the consumption of starting material (by LCMS), then added water, the aqueous phase was extracted with dichloromethane, the combined organic phases were dried over anhydrous sodium sulfate, filtered, and concentrated in vacuo, purified by prep-TLC to give the desired product 196-3 (170 mg, yield: 48%) as a brown solid.

The synthesis of 2-(4-(2-(3-cyanobenzamido)propan-2-yl)-1H-1,2,3-triazol-1-yl)ethyl methanesulfonate (196-4).

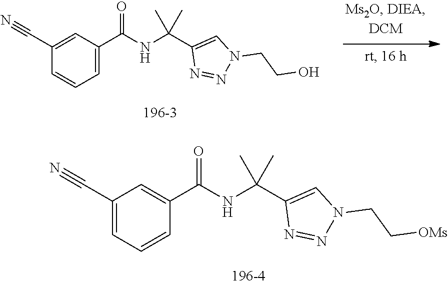 [SMILES: CC(C)(NC(=O)c1cccc(C#N)c1)c1cn(CCO)nn1 ; CC(C)(NC(=O)c1cccc(C#N)c1)c1cn(CCOS(C)(=O)=O)nn1]

To a solution of compound 196-3 (170 mg, 0.568 mmol) in DCM (15 mL) was added DIEA (303.72 mg, 2.36 mmol) and Ms₂O (128.62 mg, 0.739 mmol). The mixture was stirred for 16 h at rt. After the consumption of starting material (by LCMS), then added water, the aqueous phase was extracted with dichloromethane, the combined organic phases were dried over anhydrous sodium sulfate, filtered, and concentrated in vacuo, purified by prep-TLC to give the desired product 196-4 (150 mg, yield: 70%) as a brown solid.

The synthesis of S-2-(4-(2-(3-cyanobenzamido)propan-2-yl)-1H-1,2,3-triazol-1-yl)ethyl ethanethioate (SU20666-196)

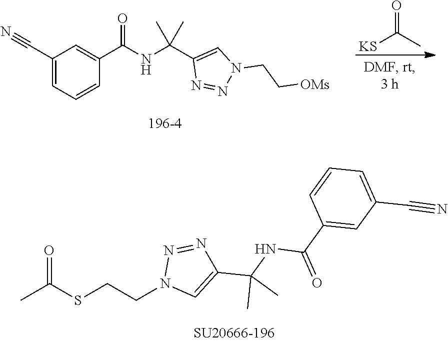

To a solution of compound 196-4 (150 mg, 0.397 mmol) in DMF (15 mL) was added Potassium thioacetate (50 mg, 0.437 mmol). The mixture was stirred for 3 h at rt, after the consumption of starting material (by LCMS), the mixture was purified by prep-TLC to give SU20666-196 (50 mg, yield: 35%) as brown oil.

LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min), Purity: 97.64%, Rt=1.780 min; MS Calcd.: 357; MS Found: 358.2 [M+H]⁺.

HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min), Purity: 96.88%, Rt=7.818 min; MS Calcd.: 357; MS Found: 358.2 [M+H]⁺.

¹H NMR (400 MHz, CDCl₃) δ 1.82 (6H, s), 2.32 (3H, s), 3.30 (2H, t, J=6.8 Hz), 4.46 (2H, t, J=6.8 Hz) , 7.06 (1H, s), 7.48 (1H, t, J=8.0 Hz), 7.54 (1H, s), 7.68-7.71 (1H, m), 7.91-7.94 (1H, m), 8.01 (1H, t, J=1.2 Hz).

The names SU20666-197-01, SP 197, and 197 all refer to the same compound having the formula:

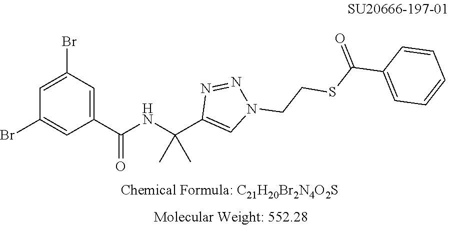 [SMILES: CC(C)(NC(=O)c1cc(Br)cc(Br)c1)c1cn(CCSC(=O)c2ccccc2)nn1]

Route for SU20666-197-01

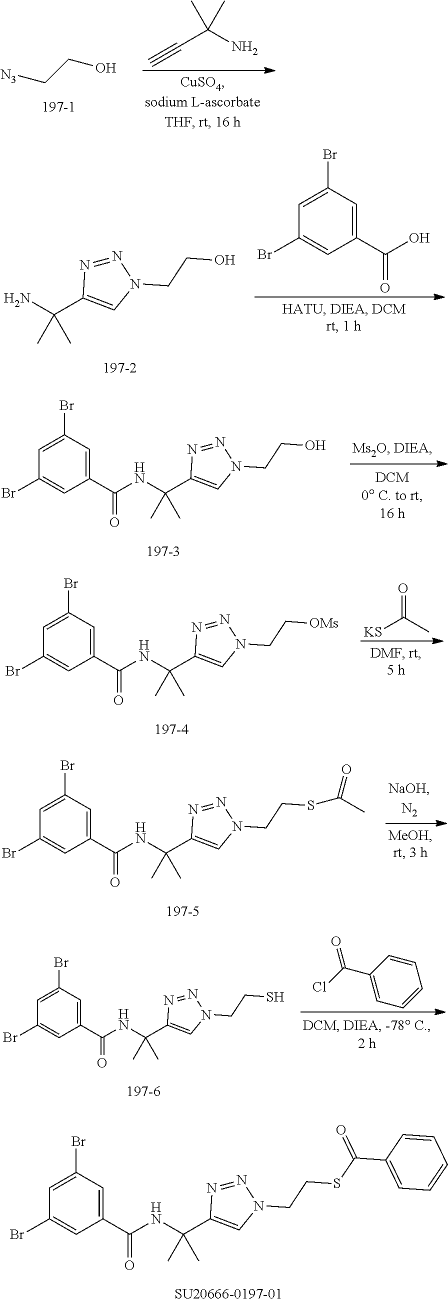

The synthesis of 2-(4-(2-aminopropan-2-yl)-1H-1,2,3-triazol-1-yl)ethanol (197-2).

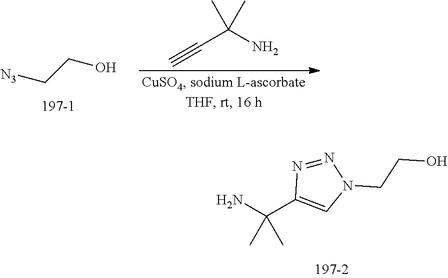

To a solution of compound 197-1 (5.0 g, 57.4 mmol) in THF (150 mL) was added Copper(II) sulfate (4.6 g, 29.0 mmol), sodium L-ascorbate (5.7 g, 29.0 mmol) and 2-methyl-3-butyn-2-amine (5.7 g, 68.9 mmol), the mixture was stirred for room temperature for overnight. After the consumption of starting material (by LCMS), the mixture was filtered and the filtrate was concentrated to give the desired product 197-2 (6.0 g, yield: 61.7%) as a brown oil.

The synthesis of 3,5-dibromo-N-(2-(1-(2-hydroxyethyl)-1H-1,2,3-triazol-4-yl)propan-2-yl)benzamide (197-3).

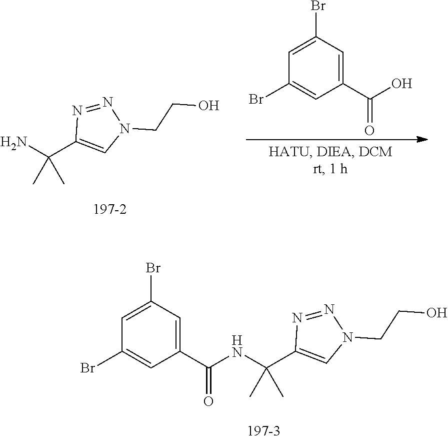 [SMILES: CC(C)(N)c1cn(CCO)nn1 ; O=C(O)c1cc(Br)cc(Br)c1 ; CC(C)(NC(=O)c1cc(Br)cc(Br)c1)c1cn(CCO)nn1]

To a solution of compound 3,5-dibromobenzoic acid (9.8 g, 35.3 mmol) in CH₂Cl₂ (150 mL) was added HATU (20.0 g, 52.9 mmol), DIEA (11.7 mL, 70.5 mmol) and 197-2 (6.0 g, 35.3 mmol). The mixture was stirred at rt for 1 h. After the consumption of starting material (detected by LCMS), the mixture was quenched with 30 mL water, and extracted with CH₂Cl₂ (60 mL×3), combined the organic layer and dried over anhydrous Na₂SO₄, filtered and concentrated in vacuo, the crude was purified by flash-chromatography (MeOH/CH₂Cl₂=5%) to give the desired product 197-3 (13.0 g, yield: 85.7%) as a white solid.

The synthesis of 2-(4-(2-(3,5-dibromobenzamido)propan-2-yl)-1H-1,2,3-triazol-1-yl)ethyl methanesulfonate (197-4).

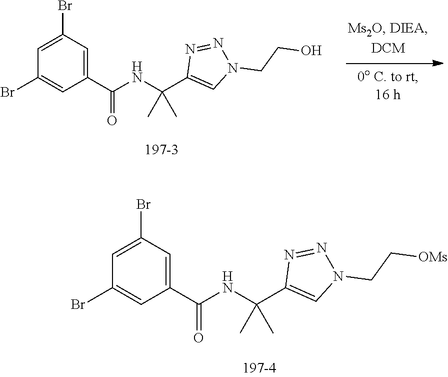 [SMILES: CC(C)(NC(=O)c1cc(Br)cc(Br)c1)c1cn(CCO)nn1 ; CC(C)(NC(=O)c1cc(Br)cc(Br)c1)c1cn(CCOS(C)(=O)=O)nn1]

To a solution of compound 197-3 (6.5 g, 30.4 mmol) in CH₂Cl₂ (100 mL) was added DIEA (5.0 mL, 60.8 mmol) and Ms₂O (4.0 g, 45.6 mmol) in 0° C. The mixture was stirred at rt for 16 h. After the reaction was finished (by LCMS), the mixture was quenched with 30 mL water, and extracted with CH₂Cl₂ (50×3 mL), combined the organic layer and dried over anhydrous Na₂SO₄, filtered and concentrated in vacuo, the crude was purified by flash-chromatography (MeOH/CH₂Cl₂=5%) to give the desired product 197-4 (5.0 g, yield: 65.1%) as a white solid.

The synthesis of S-2-(4-(2-(3,5-dibromobenzamido)propan-2-yl)-1H-1,2,3-triazol-1-yl)ethyl ethanethioate (197-5).

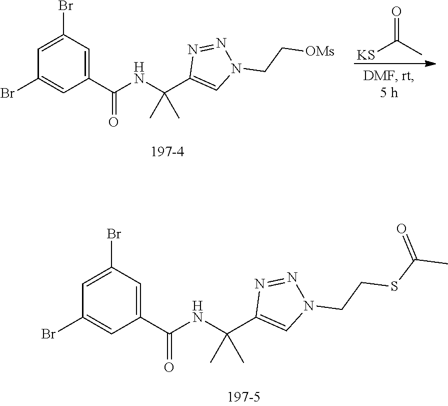

To a solution of compound 197-4 (4.9 g, 9.8 mmol) in DMF (50 mL) was added Potassium thioacetate (1.7 g, 14.7 mmol). The mixture was stirred at rt for 5 h. After the consumption of starting material (by LCMS), the mixture was quenched with 150 mL water, and extracted with EtOAc (50×3 mL), combined the organic layer and dried over anhydrous Na₂SO₄, filtered and concentrated in vacuo, the crude was purified by flash-chromatography (EtOAc/PE=55%) to give the desired product 197-5 (3.3 g, yield: 68.4%) as a white solid.

The synthesis of 3,5-dibromo-N-(2-(1-(2-mercaptoethyl)-1H-1,2,3-triazol-4-yl)propan-2-yl)benzamide (197-6).

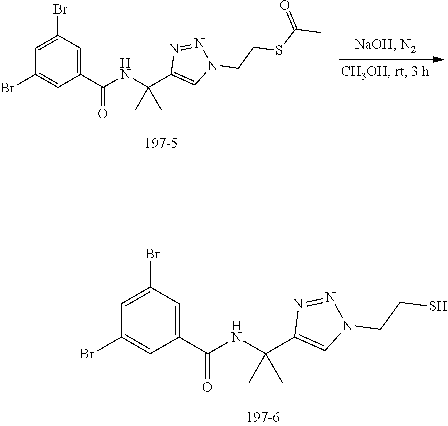 [SMILES: CC(=O)SCCn1cc(C(C)(C)NC(=O)c2cc(Br)cc(Br)c2)nn1 ; CC(C)(NC(=O)c1cc(Br)cc(Br)c1)c1cn(CCS)nn1]

To a solution of compound 197-5 (3.3 g, 6.7 mmol) in CH₃OH (50 mL) was added NaOH (535.0 mg, 2M in H₂O). The mixture was stirred at rt under N₂ protected for 3 h. After the consumption of starting material (by LCMS), the mixture was quenched with 50 mL water, the mixture was adjust pH to ˜4.0 by HCl (1.0 N), and extracted with CH₂Cl₂ (50×3 mL), combined the organic layer and dried over anhydrous Na₂SO₄, filtered and concentrated in vacuo, the crude was purified by prep-HPLC (0.05% HCOOH in water) to give the desired product 197-6 (3.0 g, yield: 99.5%) as a white solid.

The synthesis of S-2-(4-(2-(3,5-dibromobenzamido)propan-2-yl)-1H-1,2,3-triazol-1-yl)ethyl benzothioate (SU20666-197-01).

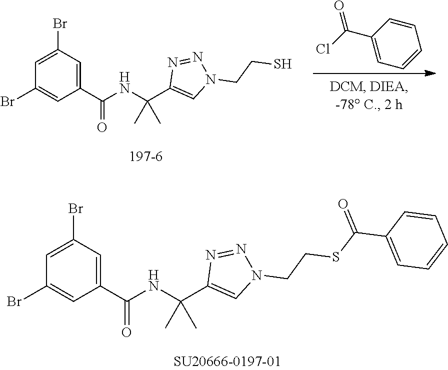 [SMILES: CC(C)(NC(=O)c1cc(Br)cc(Br)c1)c1cn(CCS)nn1 ; O=C(Cl)c1ccccc1 ; CC(C)(NC(=O)c1cc(Br)cc(Br)c1)c1cn(CCSC(=O)c2ccccc2)nn1]

To a solution of compound 197-6 (535.0 mg, 1.2 mmol) in DCM (20 mL) was added DIEA (310.0 mg, 2.4 mmol) and Benzoyl chloride (253.0 mg, 1.8 mmol). The mixture was stirred at −78° C. for 2 h. After the consumption of starting material (by LCMS), the mixture was concentrated in vacuo, the crude was purified by prep-HPLC (0.05% HCOOH in water) to give the desired product SU20666-197-01 (470 mg, yield: 71.2%) as a white solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 100.00%, Rt=2.031 min; MS Calcd.: 550.0; MS Found: 551.0 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 98.63%, Rt=10.211 min.

¹H NMR (400 MHz, CDCl₃) δ 7.93-7.96 (m, 2H), 7.81 (d, J=2.0 Hz, 2H), 7.76 (d, J=1.8 Hz, 1H), 7.59-7.63 (m, 2H), 7.47 (t, J=7.8 Hz, 2H), 6.98 (s, 1H), 4.62 (t, J=6.8 Hz, 2H), 3.56 (t, J=6.8 Hz, 2H), 1.85(s, 6H).

The names SU20666-198-01, SP 198, and 198 all refer to the same compound having the formula:

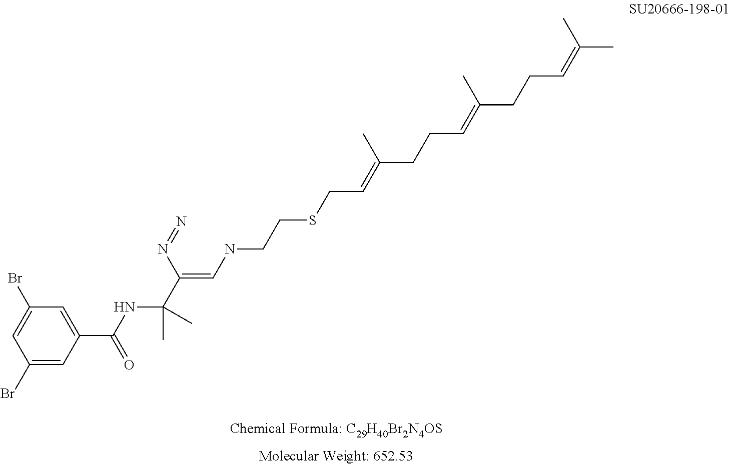

Route for SU20666-198-01

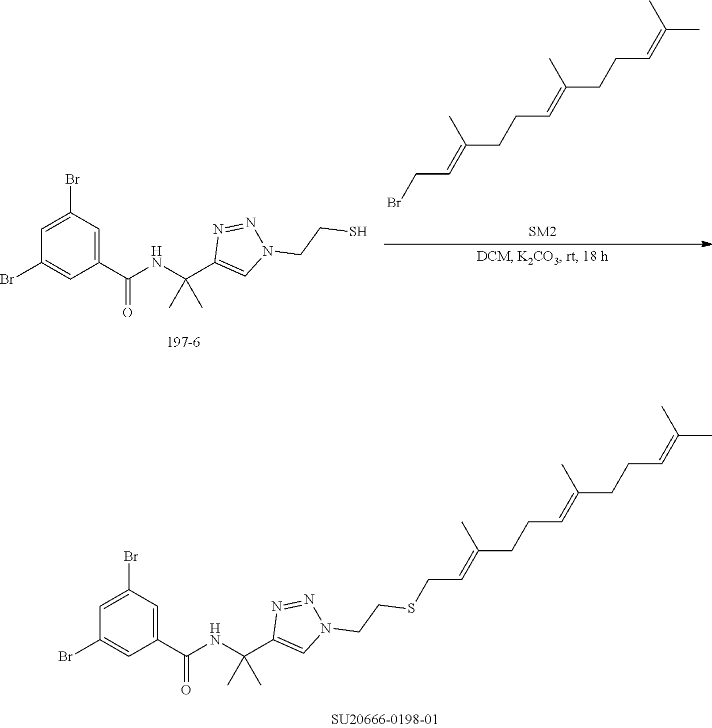 [SMILES: CC(C)(NC(=O)c1cc(Br)cc(Br)c1)c1cn(CCS)nn1 ; CC(C)=CCC/C(C)=C/CC/C(C)=C/CBr ; CC(C)=CCC/C(C)=C/CC/C(C)=C/CSCCn1cc(C(C)(C)NC(=O)c2cc(Br)cc(Br)c2)nn1]

The synthesis of 3,5-dibromo-N-(2-(1-(2-((2E,6E)-3,7,11-trimethyldodeca-2,6,10-trienylthio)ethyl)-1H-1,2,3-triazol-4-yl)propan-2-yl)benzamide (SU20666-198-01).

To a solution of compound 197-6 (535.0 mg, 1.2 mmol) in DCM (20 mL) was added K₂CO₃ (332.0 mg, 2.4 mmol), SM2 (409.0 mg, 1.4 mmol), the mixture was stirred at rt for 18 h. After the consumption of starting material (by LCMS), the mixture was quenched with H₂O (30 mL), extracted with CH₂Cl₂ (25 mL×3), combined the organic layer and dried over anhydrous Na₂SO₄, filtered and concentrated in vacuo, the crude was purified by prep-HPLC (0.05% HCCOH in H₂O) to give the desired product SU20666-0198-01 (426.0 mg, yield: 54.6%) as a white semi-solid.

LC-MS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 5% [water+0.05% TFA] and 95% [water+0.05% TFA] in 1.6 min, then under this condition for 1.4 min, finally changed to 95% [water+0.05% TFA] and 5% [CH₃CN+0.05% TFA] in 0.05 min and under this condition for 0.7 min), Purity: 98.59%, Rt=2.481 min; MS Calcd.: 650.0; MS Found: 651.2 [M+H]⁺.

HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.05% TFA] and 5% [CH₃CN+0.05% TFA] to 5% [water+0.05% TFA] and 95% [water+0.05% TFA] in 10 min, then under this condition for 5 min, finally changed to 95% [water+0.05% TFA] and 5% [CH₃CN+0.05% TFA] in 0.1 min and under this condition for 5 min), Purity: 93.48%, Rt=8.712 min.

¹H NMR (400 MHz, DMSO-d₆) δ 8.61 (s, 1H), 8.00 (s, 3H), 7.94 (s, 1H), 5.14 (t, J=7.2 Hz, 1H), 5.05 (t, J=6.4 Hz, 2H), 4.47 (t, J=6.8 Hz, 2H), 3.10 (d, J=7.6 Hz, 2H), 2.85-2.91 (m, 2H), 1.87-2.04 (m, 8H), 1.69 (s, 6H), 1.61 (d, J=8.8 Hz, 6H), 1.53 (d, J=8.8 Hz, 6H).

¹H NMR (400 MHz, CDCl₃) δ 7.82 (d, J=1.6 Hz, 2H), 7.77 (t, J=1.6 Hz, 1H), 7.62 (s, 1H), 7.00 (s, 1H), 5.21 (t, J=7.6 Hz, 1H), 5.08 (t, J=5.6 Hz, 2H), 4.52 (t, J=6.8 Hz, 2H), 3.14 (d, J=7.6 Hz, 2H), 2.95 (t, J=6.8 Hz, 2H), 1.94-2.11 (m, 8H), 1.87 (s, 6H), 1.67 (d, J=6.4 Hz, 6H), 1.59 (d, J=3.2 Hz, 6H).

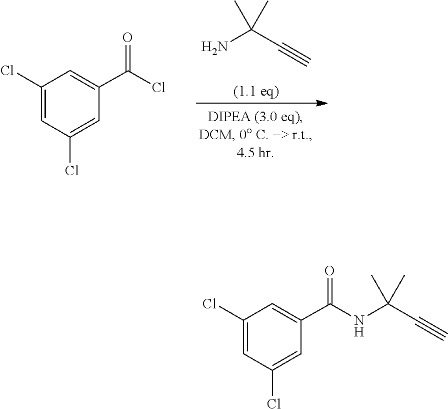

3,5-dichloro-N-(2-methylbut-3-yn-2-yl)benzamide (Pronamide, Intermediate #1)

In a round bottom flask, 3,5-dichlorobenzoyl chloride (3 g, 14.32 mmol, 1.0 eq.) dissolved in dichloromethane (100 mM) in an ice bath under nitrogen. Then 2-methyl-3-butyn-2-amine (1.31 g, 15.70 mmol, 1.66 ml, 1.1 eq.) via syringe followed by diisopropylethylamine (5.55 g, 42.96 mmol, 7.48 ml, 3.0 eq.). The reaction flask was removed the ice bath and allowed to stir for 4.5 hours. The reaction was monitored by TLC using 30% ethyl acetate in hexanes (3:7 EtOAc/Hex). Upon completion, the reaction mixture was diluted with DCM, washed with 1M HCl, brine, dried over anhydrous sodium sulfate, and concentrate by rotovap. The crude material was purified by column chromatography 3:7 EtOAc to Hex. At large scales, product was recrystallized using 10% EtOAc in Hex. The product was a white solid. 90% (2.29 g). ¹H NMR (400 mHz, CDCl₃, TMS) δ 7.76 (d, J=1.88, 2H), 7.48 (t, J=1.88, 1H), 6.09 (s, 1H), 2.41 (s, 1H), 1.76 (s, 6H).

GWL3.008

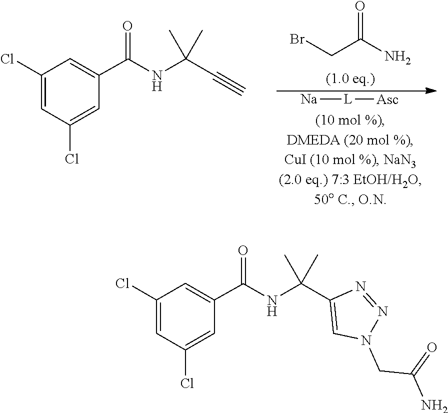

(B) N-(2-(1-(2-amino-2-oxoethyl)-1H-1,2,3-triazol-4-yl)propan-2-yl)-3,5-dichlorobenzamide (GWL3.008, Intermediate #2)

In a round bottom flask, pronamide (intermediate #1) (371 mg, 1.450 mmol, 1.0 eq.), 2-bromoacetamide (200 mg, 1.450 mmol, 1.0 eq.), copper(I)iodide (28 mg, 0.145 mmol, 0.1 eq.), sodium L-ascorbate (29 mg, 0.145 mmol, 0.1 eq.), and sodium azide (189 mg, 2.900 mmol, 0.1 eq.) were dissolved 15 ml of 7:3 ethanol to deionized water. Then, N, N′-dimethylethylenediamine (26 mg, 0.03 ml, 0.290 mmol, 0.2 eq.) was added via syringe. The reaction mixture was placed in an oil bath at 50° C. and allowed to mix overnight. The reaction was monitored by TLC with 10% methanol in dichloromethane. Upon completion, the reaction mixture was diluted with deionized water and extracted four times with ethyl acetate. The organic extracts were combined, washed with brine, and dried over anhydrous sodium sulfate. The crude material was purified by column chromatography with 5% methanol in ethyl acetate. The product was a white solid. 65% (336 mg). ¹H NMR (400 mHz, DMSO-d₆, TMS) δ 8.60 (s, 1H), 7.88 (s, 1H), 7.84 (d, J=1.92, 2H), 7.79 (t, J=1.88, 1H), 7.64 (s, 1H), 7.36 (s, 1H), 4.99 (s, 2H), 1.71 (s, 6H).

SP 150

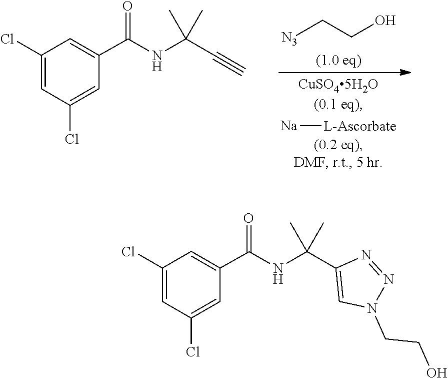

(C) 3,5-dichloro-N-(2-(1-(2-hydroxyethyl)-1H-1,2,3-triazol-4-yl)propan-2-yl)benzamide (SP 150, Intermediate #6)

In a round bottom flask, pronamide (intermediate #1) (1.40 g, 5.420 mmol, 1.0 eq.), copper(II) sulfate pentahydrate (136 mg, 0.542 mmol, 0.1 eq.), and sodium-L-ascorbate (215 mg, 1.084 mmol, 0.2 eq.) were dissolved in dimethylformamide (27.1 ml, 200 mM) under nitrogen. Then, 2-azidoethanol (472 mg, 5.420 mmol, 0.4 ml, 1.0 eq.) via syringe. The reaction was monitored by TLC with 7:3 EtOAc/Hex. Upon completion, the reaction solution was diluted with deionized water and extracted three times with ethyl acetate. The organic extracts were combined, washed with brine, dried over anhydrous sodium sulfate, and concentrated. The crude material was purified by column chromatography. A solvent system of 7:3 EtOAc/Hex was used to wash away the starting material followed by 10% methanol in dichloromethane to elute the product. The product was a white solid. 93% (1.74 g). ¹H NMR (400 mHz, CDCl₃, TMS) δ 7.66 (s, 1H), 7.62 (d, J=1.72, 2H), 7.46 (t, J=1.68, 1H), 6.97 (s, 1H), 4.49 (t, J=4.80, 2H), 4.08 (t, J=4.24, 2H), 2.50 (s, 1H), 1.87 (s, 6H).

GWL2.131

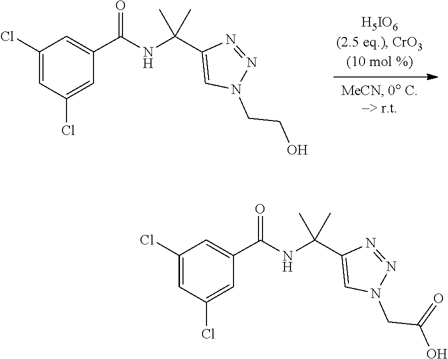 [SMILES: CC(C)(NC(=O)c1cc(Cl)cc(Cl)c1)c1cn(CCO)nn1 ; CC(C)(NC(=O)c1cc(Cl)cc(Cl)c1)c1cn(CC(=O)O)nn1]

(D) 2-(4-(2-(3,5-dichlorobenzamido)propan-2-yl)-1H-1,2,3-triazol-1-yl)acetic acid (GWL2.131, Intermediate #7)

An solution of 6.67 mM Cr0₃ (6 mg, 0.06 mmol) and 243 mM periodic acid (500 mg, 2.194 mmol) was prepared in acetonitrile (9 mL). In a round bottom flask, intermediate #6 (100 mg, 0.2914 mmol) was dissolved in acetonitrile (2.3 mL). Under nitrogen, the reaction mixture was placed in an ice bath nitrogen and 3 mL of the CrO₃/H₅IO₆ solution was added via syringe. The reaction mixture stirred for 1 hour at 0° C. and then allowed to stir at room temperature for 1 hour. The reaction was monitored by TLC (100% EtOAc). When complete, the reaction was quenched with solution of Na₂HPO₄ (87 mg) in water (3 mL). The pH of the solution was adjusted to between 3 and 4. The solution was transferred to a separatory funnel and extracted five times with ethyl acetate. The organic extracts were washed with brine, dried over anhydrous sodium sulfate, and concentrated. The crude material was purified by column chromatography with 3% acetic acid, 5% methanol in dichloromethane. The purified product was a white solid. 15.3% (16 mg). ¹H NMR (400 mHz, DMSO-d₆, TMS) δ 8.59 (s, 1H), 7.86 (s, 1H), 7.84 (d, J=1.84) 2H, 7.78 (t, J=1.84, 1H), 4.98 (s, 2H), 1.71 (s, 6H).

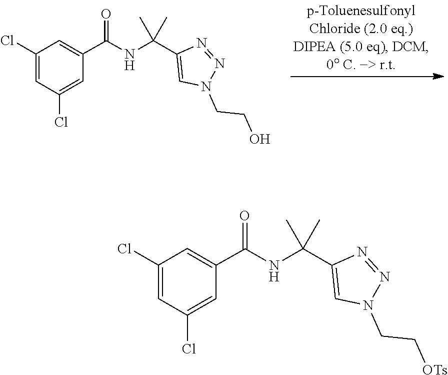 [SMILES: CC(C)(NC(=O)c1cc(Cl)cc(Cl)c1)c1cn(CCO)nn1 ; Cc1ccc(S(=O)(=O)OCCn2cc(C(C)(C)NC(=O)c3cc(Cl)cc(Cl)c3)nn2)cc1]

(E) 2-(4-(2-(3,5-dichlorobenzamido)propan-2-yl)-1H-1,2,3-triazol-1-yl)ethyl 4-methylbenzenesulfonate (Intermediate #8)

In a round bottom flask, intermediate #6 (400 mg, 1.166 mmol, 1.0 eq.) and tosyl-chloride (444 mg, 2.331 mmol, 2.0 eq.) were dissolved in dichloromethane (12 ml, 100 mM) in an ice bath under nitrogen. Then, diisopropylethylamine (753 mg, 5.828 mmol, 1 ml, 5.0 eq.) was added via syringe. The ice bath was removed and the reaction was allowed to proceed for 5 hours. The reaction was monitored by TLC with 1:1 EtOAc/Hex. Upon completion, the reaction mixture was diluted reaction mixture with dichloromethane, washed with 1M HCl, washed with brine, dried over anhydrous sodium sulfate, and concentrated by rotary evaporator. The crude material was purified by column chromatography with 1:1 EtOAc/Hex. The product was a white solid. 73% (422 mg). ¹H NMR (400 mHz, CDCl₃, TMS) δ 7.72 (d, J=8.36, 1H), 7.62 (d, J=1.88, 1H), 7.60 (s, 1H), 7.46 (t, J=1.88, 1H), 7.33 (d, J=8.04, 2H), 6.90 (s, 1H), 4.64 (t, J=5.00, 2H), 4.41 (t, J=5.4, 2H), 2.44 (s, 3H), 1.86 (s, 6H).

SP 87

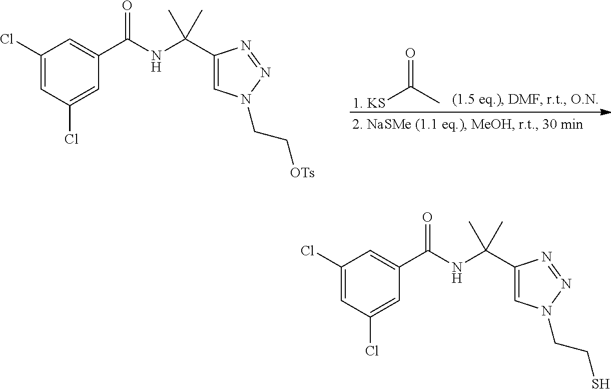

(F) 3,5-dichloro-N-(2-(1-(2-mercaptoethyl)-1H-1,2,3-triazol-4-yl)propan-2-yl)benzamide (SP 87, Intermediate #9)

In a round bottom flask, dissolve intermediate #8 (600 mg, 1.206 mmol, 1.0 eq.) and potassium thioacetate (350 mg, 2.016 mmol, 2.5 eq.) in DMF (12 ml, 100 mM) under nitrogen. The reaction was monitored by TLC with 1:1 EtOAc/Hex. Upon completion, the reaction mixture was diluted with 1:1 saturate NaHCO₃/H₂O and extracted three times with ethyl acetate. The organic extracts were combined, washed with brine, dried over anhydrous sodium sulfate, and concentrated. The recovered material was used in the next step and assumed a quantitative yield. The recovered material was dissolved in methanol (12 ml, 100 mM). A 1M solution of sodium thiomethoxide in methanol was prepared. Sodium thiomethoxide (250 mg, 3.618 mmol, 3.6 ml, 3.0 eq.) was added via syringe. The reaction progress was monitored by TLC in 1:1 EtOAc/Hex. Upon completion, the reaction mixture was concentrated. The crude material was purified by flash chromatography using 5% ethyl acetate in dichloromethane. The product was white solid. 88% (383 mg). ¹H NMR (400 mHz, DMSO-d₆, TMS) δ 8.60 (s, 1H), 7.97 (s, 1H), 7.83 (d, J=1.92, 2H), 7.89 (t, J=1.92, 1H), 4.45 (t, J=6.96, 2H), 2.94 (q, J=7.44, 2H), 2.43 (t, J=8.12, 1H), 1.70 (s, 6H).

GWL2.97

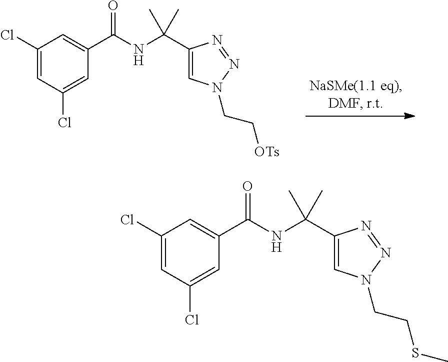

(G) 3,5-dichloro-N-(2-(1-(2-(methylthio)ethyl)-1H-1,2,3-triazol-4-yl)propan-2-yl)benzamide (GWL2.97, Intermediate #10)

Intermediate #8 (150 mg, 0.3016 mmol, 1.0 eq.) and sodium thiomethoxide (23 mg, 0.3317 mmol, 1.1 eq., Sigma Aldrich) were placed in a round bottom flask with a stir bar. Dimethylformamide (6.0 mL, 50 mM, Acros Organic) was added via syringe under nitrogen balloon. The reaction mixture was allowed to stir overnight at room temperature. The completion of the reaction was confirmed by TLC (7:3 EtOAc/Hex). The reaction mixture was diluted with deionized water and extracted three times with ethyl acetate. The organic extracts were combined, washed with brine, dried of anhydrous sodium sulfate. The crude material was purified by column chromatography (1:1 EA/Hex). Product was a white solid. 75.4%, 85 mg. ¹H NMR (400 mHz, CDCl₃, TMS) δ 7.62 (d, J=1.28, 2H) 7.61 (s, 1H), 7.46 (t, J=1.64, 1H), 6.97 (s, 1H), 4.55 (t, J=6.92, 2H), 3.00 (t, J=6.96, 2H), 2.08 (s, 3H), 1.88 (s, 6H).

GWL2.143

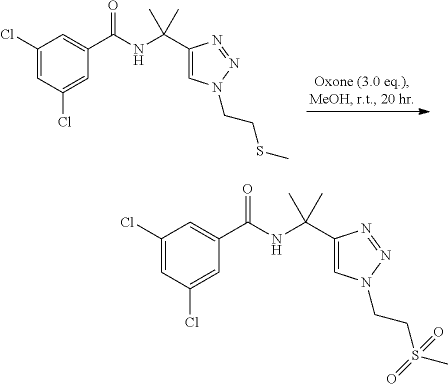

(H) 3,5-dichloro-N-(2-(1-(2-(methylsulfonyl)ethyl)-1H-1,2,3-triazol-4-yl)propan-2-yl)benzamide (GWL2.143, Intermediate #11)

A solution of 350 mM Oxone (212 mg, 1.4032 mmol) in methanol (4 mL) was prepared. #87(SMe) (87.3 mg, 0.2339 mmol, 1.0 eq.) was dissolved in 2.7 mL of methanol in a round bottom flask. Under nitrogen, 2 mL of the Oxone solution was added to the intermediate #10 solution via syringe. The reaction mixture was allowed to stir at room temperature overnight (20 hr.). The reaction progress was monitored by TLC (7:3 EtOAc/Hex). The reaction mixture was concentrated, then diluted with deionized water. The aqueous layer was extracted four times with ethyl acetate. The organic extracts were combined, washed with brine, dried with anhydrous sodium sulfate, and concentrated. The crude material was purified by column chromatography with 1:1 EtOAc/Hex and 7:3 EtOAc/Hex. Product was a white solid. 77%, 73.6 mg. ¹H NMR (400 mHz, DMSO-d₆, TMS) δ 8.63 (s, 1H), 8.04 (s, 1H), 7.83 (d, J=1.92, 2H), 7.79 (t, J=1.92, 1H), 4.74 (t, J=6.92, 2H), 3.78 (t, J=7.12, 2H), 2.88 (s, 3H), 1.69 (s, 6H).

GWL2.137

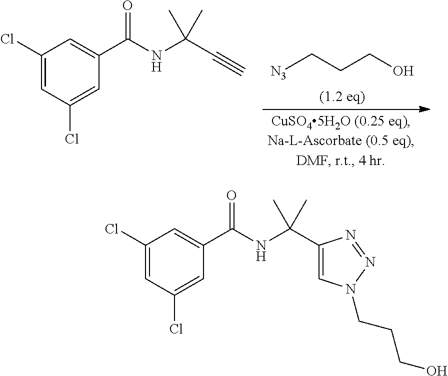

(J) 3,5-dichloro-N-(2-(1-(3-hydroxypropyl)-1H-1,2,3-triazol-4-yl)propan-2-yl)benzamide (GWL2.137, Intermediate #3)

Intermediate #3 was prepared using the same procedure as #87(OH) but used 3-azidopropanol instead of 2-azidoethanol. The product was a white solid. 88% (610 mg). ¹H NMR (400 mHz, CDCl₃, TMS) δ 6.62 (d, J=1.88, 2H), 7.57 (s, 1H), 7.46 (t, J=1.88, 1H), 6.97 (s, 1H), 4.52 (t, J=6.80, 2H), 3.67 (t, J=5.80, 2H), 2.16 (m, J=6.60, 5.98, 2H), 1.87 (s, 6H), 1.77 (s, 1H).

GWL2.129

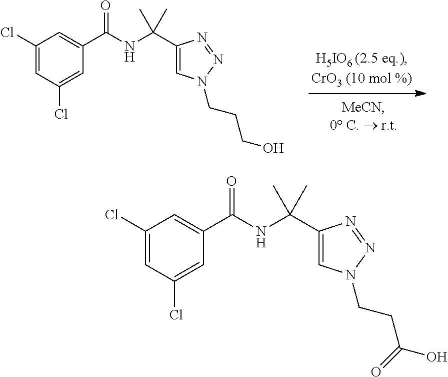

(J) 3-(4-(2-(3,5-dichlorobenzamido)propan-2-yl)-1H-1,2,3-triazol-1-yl)propanoic acid (GWL2.129, Intermediate #4)

This compound was prepared using the same procedure as #87(COOH). The purified product was a white solid. 21% (66.8 mg). ¹H NMR (400 mHz, DMSO-d₆, TMS) δ 8.58 (s, 1H), 7.92 (s, 1H), 7.83 (d, J=1.88, 2H), 7.78 (t, J=1.84, 1H), 7.49 (t, J=6.92, 2H), 2.85 (t, J=6.88, 2H), 1.68 (s, 6H).

GWL2.154

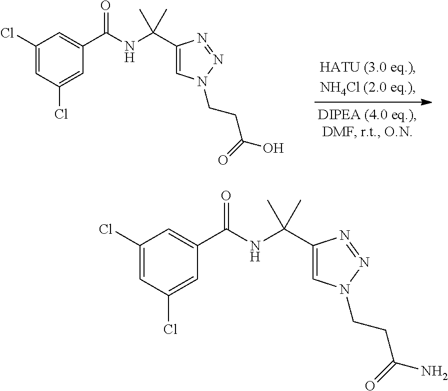

(K) N-(2-(1-(3-amino-3-oxopropyl)-1H-1,2,3-triazol-4-yl)propan-2-yl)-3,5-dichlorobenzamide (GWL2.154, Intermediate #5)

In a round bottom flask, intermediate #4 (100 mg, 0.269 mmol, 1.0 eq.), HATU (307 mg, 0.808 mmol, 3.0 eq.), and ammonium chloride (29 mg, 0.539 mmol, 2.0 eq.) was dissolved in dimethylformamide (2.7 ml, 100 mM) under nitrogen. Diisopropylethylamine (139 mg, 0.2 ml, 1.078 mmol, 4.0 eq.) was added via syringe. The reaction mixture was stirred at room temperature overnight. The reaction progress was monitored by TLC with 5% methanol in dichloromethane. Upon completion, the reaction solution was diluted with ethyl acetate, washed with 1 M HCl, 1:1 sat. sodium biocarbonate to deionized water, and brine. The crude organic mixture was dried over anhydrous sodium sulfate and concentrated by rotary evaporator. The crude material was purified by column chromatography with dichloromethane and 5% methanol in dichloromethane. The product was a white solid. 47% (48 mg). ¹H NMR (400 mHz, DMSO-d₆, TMS) δ 8.58 (s, 1H), 7.87 (s, 1H), 7.84 (d, J=1.88, 2H), 7.78 (t, J=1.88, 1H), 7.41 (s, 1H), 6.95 (s, 1H), 4.47 (t, J=7.08, 2H), 2.67 (t, J=7.08, 2H), 1.68 (s, 6H).

GWL2.171

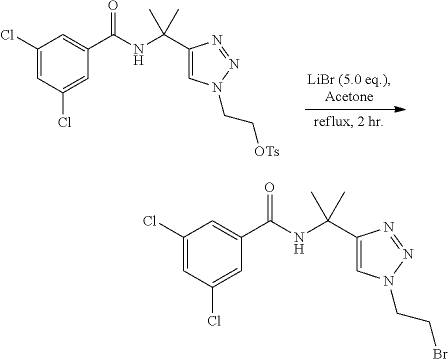

N-(2-(1-(2-bromoethyl)-1H-1,2,3-triazol-4-yl)propan-2-yl)-3,5-dichlorobenzamide (GWL2.171)

In a round bottom flask, intermediate #8 (200 mg, 0.402 mmol, 1.0 eq.) and lithium bromide (175 mg, 2.010 mmol, 5.0 eq.) were dissolved in acetone. A reflux condenser was assembled and the reaction was placed in an oil bath. The reaction was allowed to stir for 2 hours under nitrogen. The reaction progress was monitored by TLC with 1:1 EtOAc/Hex. Upon completion of the reaction, the solvent was removed by rotary evaporator. The crude material was purified by column chromatography with 3:7 EtOAc/Hex. The product was a white solid. 91% (149 mg). ¹H NMR (400 mHz, DMSO-d₆, TMS) δ 8.61 (s, 1H), 8.00 (s, 1H), 7.84 (d, J=1.92, 2H), 7.89 (t, J=1.92, 2H), 4.73 (t, J=6.12, 2H), 3.90 (t, J=6.12, 2H), 1.70 (s, 6H).

GWL3.001

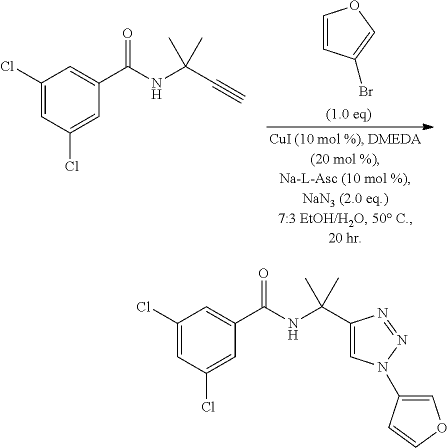 [SMILES: C#CC(C)(C)NC(=O)c1cc(Cl)cc(Cl)c1 ; Brc1ccoc1 ; CC(C)(NC(=O)c1cc(Cl)cc(Cl)c1)c1cn(-c2ccoc2)nn1]

3,5-dichloro-N-(2-(1-(furan-3-yl)-1H-1,2,3-triazol-4-yl)propan-2-yl)benzamide (GWL3.001)

In a round bottom flask, pronamide (intermediate #1) (174 mg, 0.680 mmol, 1.0 eq.), 3-bromofuran (100 mg, 0.680 mmol, 1.0 eq.), copper(I)iodide (13 mg, 0.068 mmol, 0.1 eq.), sodium L-ascorbate (14 mg, 0.068 mmol, 0.1 eq.), and sodium azide (88 mg, 1.361 mmol, 2.0 eq.) were dissolved 6.8 ml of 7:3 ethanol to deionized water. Then, N, N′-dimethylethylenediamine (12 mg, 0.01 ml, 0.136 mmol, 0.2 eq.) was added via syringe. The reaction mixture was placed in an oil bath at 50° C. and allowed to mix for 20 hours. The reaction was monitored by TLC with 3:7 EtOAc/Hex. Upon completion, the reaction mixture was diluted with deionized water and extracted three times with ethyl acetate. The organic extracts were combined, washed with brine, and dried over anhydrous sodium sulfate. The crude material was purified by column chromatography with 1:1 EtOAc/Hex. The product was a white solid. 10% (36 mg). ¹H NMR (400 mHz, DMSO-d₆, TMS) δ 8.71 (s, 1H), 8.46 (s, 1H), 8.39 (s, 1H), 7.87 (d, J=1.84, 3H), 7.79 (t, J=1.76, 1H), 7.11 (d, J=1.44, 1H), 1.74 (s, 6H).

GWL2.190

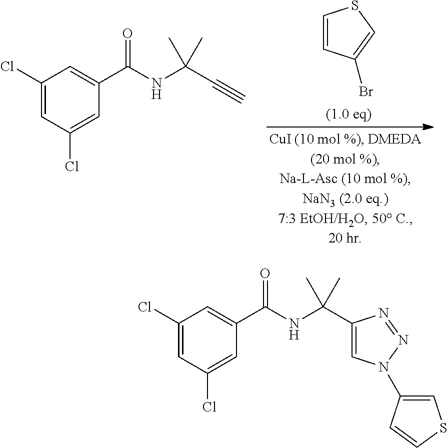 [SMILES: C#CC(C)(C)NC(=O)c1cc(Cl)cc(Cl)c1 ; Brc1ccsc1 ; CC(C)(NC(=O)c1cc(Cl)cc(Cl)c1)c1cn(-c2ccsc2)nn1]

3,5-dichloro-N-(2-(1-(thiophen-3-yl)-1H-1,2,3-triazol-4-yl)propan-2-yl)benzamide (GWL2.190)

In a round bottom flask, pronamide (intermediate #1) (122 mg, 0.476 mmol, 1.0 eq.), 3-iodothiophene (100 mg, 0.476 mmol, 1.0 eq.), copper(I)iodide (9 mg, 0.048 mmol, 0.1 eq.), sodium L-ascorbate (10 mg, 0.048 mmol, 0.1 eq.), and sodium azide (62 mg, 0.952 mmol, 2.0 eq.) were dissolved 4.7 ml of 7:3 ethanol to deionized water. Then, N, N′-dimethylethylenediamine (12 mg, 0.01 ml, 0.136 mmol, 0.2 eq.) was added via syringe. The reaction mixture was placed in an oil bath at 50° C. and allowed to mix for 20 hours. The reaction was monitored by TLC with 1:1 EtOAc/Hex. Upon completion, the reaction mixture was diluted with deionized water and extracted three times with ethyl acetate. The organic extracts were combined, washed with brine, and dried over anhydrous sodium sulfate. The crude material was purified by column chromatography with 3:7 EtOAc/Hex. The product was a white solid. 86% (157 mg). ¹H NMR (400 mHz, DMSO-d₆, TMS) δ 8.71 (s, 1H), 8.59 (s, 1H), 8.00 (dd, J=1.36, 1.80, 1.40, 1H), 7.87 (d, J=1.88, 2H), 7.79 (t, J=1.88, 1H), 7.78 (m, J=2.00, 5.28, 1H), (dd, J=1.36, 5.24, 1.36, 1H) 1.75 (s, 6H).

UNI6-001

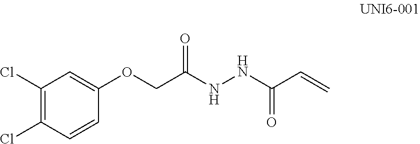 [SMILES: C=CC(=O)NNC(=O)COc1ccc(Cl)c(Cl)c1]

Route for UNI6-001

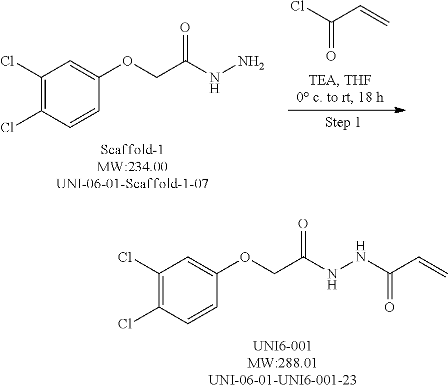

Step-1: Synthesis of N′-(2-(3,4-dichlorophenoxy)acetyl)acrylohydrazide

To a solution of Scaffold-1 (0.20 g, 0.00085 mol) in THF (2 mL) was added TEA (0.17 mL, 0.00128 mol) at 0° C. Acrolyl chloride (0.06 mL, 0.000854 mol) was drop wise added to the reaction mixture at 0° C. The reaction mixture was stirred at 0° C. for 4 h and further at room temperature for 14 h. Reaction was monitored by TLC (5% methanol in chloroform). The resulting reaction mixture was poured into water (20 mL) and extracted with Ethyl acetate (2×30 mL). The combined organic phase was washed with brine solution (15 mL), dried over sodium sulfate, filtered and concentrated under vacuum. The obtained crude material was purified by flash chromatography (product was eluated using 35% Ethyl acetate in Hexane) yielding title compound (0.022 g, 000076 mol, 9%).

¹H NMR (400 MHz, DMSO-d6) δ ppm: 10.29 (s, 1H), 10.20 (s, 1H), 7.56 (d, J=8.8 Hz, 1H), 7.28 (d, J=2.8 Hz, 1H), 7.01 (dd, J=8.8, 2.8 Hz, 1H), 6.32-6.17 (m, 2H), 5.73 (dd, J=9.6, 2.4 Hz, 1H) 4.73 (s, 2H).

LCMS Method: UPLC_2A, O2H METHOD_A_0, RT-1.914 min. MS: 288.96 (M+1).

Scaffold-1

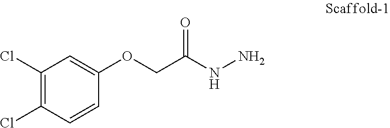 [SMILES: NNC(=O)COc1ccc(Cl)c(Cl)c1]

Route for Scaffold-1

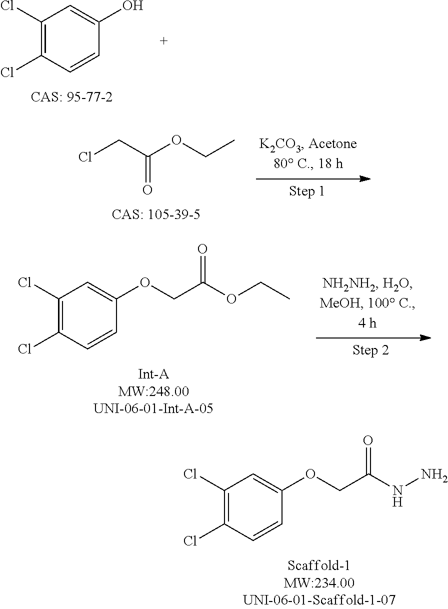

Step-1: Synthesis of ethyl 2-(3,4-dichlorophenoxy)acetate

To a solution of 3,4-dichloro phenol (15.0 g, 0.0920 mol) in Acetone (150 mL) was added K₂CO₃ (17.8 g, 0.128 mol) at room temperature. Ethyl chloro acetate (10.6 mL, 0.101 mol) was drop wise added to the reaction mixture at room temperature. The reaction mixture was heated at 80° C. for 18 h. Reaction was monitored by TLC (10% ethyl acetate in hexane). The resulting reaction mixture was allowed to cool to room temperature, poured into water (300 mL) and extracted with Ethyl acetate (4×100 mL). The combined organic phase was washed with brine solution (50 mL), dried over sodium sulfate, filtered and concentrated under vacuum yielding ethyl 2-(3,4-dichlorophenoxy)acetate (23 g, 0.0927 mol, 100%). This material was directly used for next step without any further purification.

¹H NMR (400 MHz, DMSO-d6) δ ppm: 7.52 (d, J=9.2 Hz, 1H), 7.26 (d, J=2.8 Hz, 1H), 6.97 (dd, J=8.8, 2.8 Hz, 1H), 4.85 (s, 2H), 4.19-4.13 (m, 2H), 1.20 (t, J=8.4 Hz, 3H).

Step-2: Synthesis of 2-(3,4-dichlorophenoxy)acetohydrazide

To a solution of ethyl 2-(3,4-dichlorophenoxy)acetate (1.0 g, 0.00403 mol) in Ethanol (10 mL) was added Hydrazine hydrate solution (1 mL, 0.0201 mol) at room temperature. The reaction mixture was heated at 80° C. for 4 h. Reaction was monitored by TLC (5% methanol in chloroform). We have carried out another 14 batches with same scale and worked up together. The resulting reaction mixture was poured into water (450 mL) and extracted with Ethyl acetate (3×200 mL). The combined organic phase was washed with brine solution (100 mL), dried over sodium sulfate, filtered and concentrated under vacuum. The obtained solid material was purified by trituration using diethyl ether (2×25 mL) yielding 2-(3,4-dichlorophenoxy)acetohydrazide (8.4 g, 0.0358 mol, 59%).

¹H NMR (400 MHz, DMSO-d6) δ ppm: 9.38 (s, 1H), 7.52 (d, J=9.2 Hz, 1H), 7.25 (d, J=2.8 Hz, 1H), 6.98 (dd, J=8.8, 2.8 Hz, 1H), 4.54 (s, 2 H), 4.35 (s, 2H).

LCMS Method: UPLC_2A, O2H METHOD_A_3, RT-1.850 min. MS: 235.04 (M+1).

UNI6-002

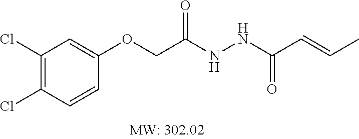 [SMILES: C/C=C/C(=O)NNC(=O)COc1ccc(Cl)c(Cl)c1]

Route for UNI6-002

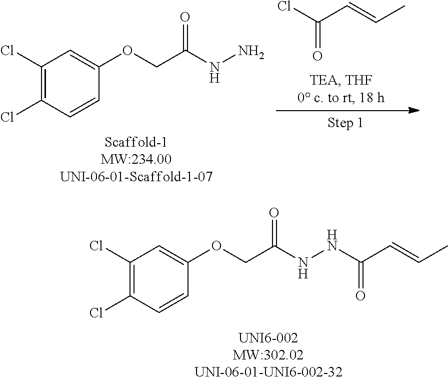

Step-1: Synthesis of (E)-N′-(2-(3,4-dichlorophenoxy)acetyl)but-2-enehydrazide

To a solution of Scaffold-1 (0.50 g, 0.0021 mol) in THF (5 mL) was added TEA (0.44 mL, 0.00320 mol) at 0° C. Crotonyl chloride (0.2 mL, 0.00213 mol) was drop wise added to the reaction mixture at 0° C. The reaction mixture was stirred at 0° C. for 4 h and further at room temperature for 14 h. Reaction was monitored by TLC (5% methanol in chloroform). The resulting reaction mixture was poured into water (30 mL) and extracted with Ethyl acetate (3×15 mL). The combined organic phase was washed with brine solution (10 mL), dried over sodium sulfate, filtered and concentrated under vacuum. The obtained crude material was purified by flash chromatography (product was eluting using 57% Ethyl acetate in Hexane) yielding title compound (0.052 g, 000172 mol, 8%).

¹H NMR (400 MHz, DMSO-d6) δ ppm: 10.19 (s, 1H), 9.96 (s, 1H), 7.55 (d, J=8.8 Hz, 1H), 7.26 (d, J=2.8 Hz, 1H), 7.01 (dd, J=8.8, 2.8 Hz, 1H), 6.78-6.69 (m, 1H), 5.96 (dd, J=15.2 , 1.6 Hz, 1H), 4.68 (s, 2H), 1.82(dd, J=6.8, 1.6 Hz, 3H).

LCMS Method: UPLC_2A, O2H METHOD_A_1, RT-2.009 min. MS: 303.01 (M+1).

UNI6-003

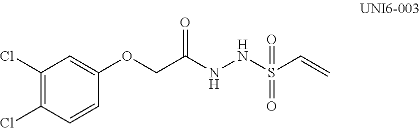 [SMILES: C=CS(=O)(=O)NNC(=O)COc1ccc(Cl)c(Cl)c1]

Route for UNI6-003

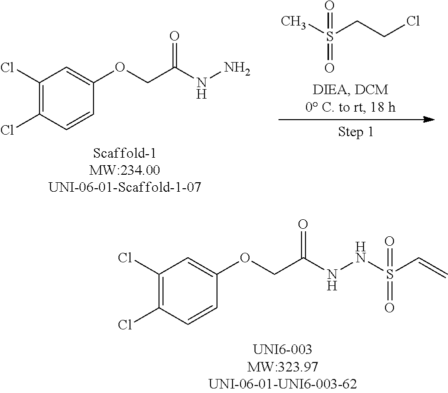

Synthesis of N′-(2-(3,4-dichlorophenoxy)acetyl)ethenesulfonohydrazide

To a solution of Scaffold-1 (0.5 g, 0.00213 mol) in DCM (6.5 mL) was added N,N-Diisopropyl ethyl amine (0.28 mL, 0.00341 mol) at 0° C. 2-Chloro ethane sulfonyl chloride (0.56 mL, 0.00256 mol) was drop wise added to the reaction mixture at 0° C. The reaction mixture was stirred at 0° C. for 3 h and further at room temperature for 15 h. Reaction was monitored by TLC (5% methanol in chloroform). The resulting reaction mixture was poured into water (40 mL) and extracted with Ethyl acetate (3×25 mL). The combined organic phase was washed with brine solution (10 mL), dried over sodium sulfate, filtered and concentrated under vacuum. The obtained crude material was purified by flash chromatography (product was eluting using 82% Ethyl acetate in Hexane) yielding titled compound (0.33 g, 0.00101 mol). The compound was further purified by Prep-HPLC purification using 10mm Ammonium bicarbonate in ACN:Water (0.030 g, 0.000092 mol). The isolated solid material was further purified by trituration using Methanol (1 mL) yielding titled compound (0.019 g, 0.000058 mol, 3%).

¹H NMR (400 MHz, DMSO-d6) δ ppm: 10.47 (s, 1H), 9.67 (s, 1H), 7.54 (d, J=8.8 Hz, 1H), 7.20 (d, J=2.8 Hz, 1H), 6.95 (dd, J=8.8 , 1.6 Hz, 1H), 6.65-6.59 (m, 1H), 6.08-5.99 (m, 2H), 4.65 (s, 2H).

LCMS Method: UPLC_3AA, O2H METHOD_A_2, RT-2.632 min. MS: 323.12 (M−1).

UNI6-0005

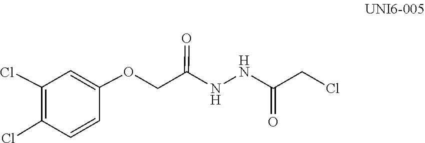 [SMILES: O=C(CCl)NNC(=O)COc1ccc(Cl)c(Cl)c1]

Route for UNI6-005

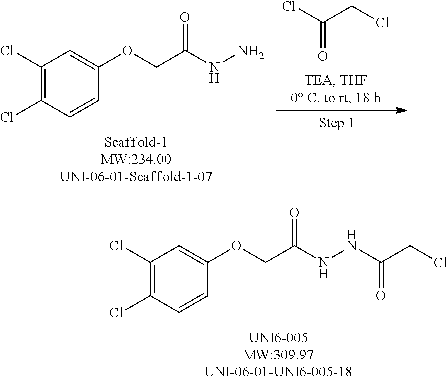 [SMILES: NNC(=O)COc1ccc(Cl)c(Cl)c1 ; O=C(Cl)CCl ; O=C(CCl)NNC(=O)COc1ccc(Cl)c(Cl)c1]

Step-1: Synthesis of 2-chloro—N′-(2-(3,4-dichlorophenoxy)acetyl)acetohydrazide

To a solution of Scaffold-1 (0.15 g, 0.000641 mol) in THF (1.5 mL) was added TEA (0.13 mL, 0.000961 mol) at 0° C. Chloro acetyl chloride (0.05 mL, 0.000641 mol) was drop wise added to the reaction mixture at 0° C. The reaction mixture was stirred at 0° C. for 4 h and further at room temperature for 14 h. Reaction was monitored by TLC (5% methanol in chloroform). The resulting reaction mixture was poured into water (30 mL) and extracted with Ethyl acetate (2×40 mL). The combined organic phase was washed with brine solution (20 mL), dried over sodium sulfate, filtered and concentrated under vacuum. The obtained crude material was purified by flash chromatography (product was eluated using 45% Ethyl acetate in Hexane) yielding titled compound (0.089 g, 000287 mol, 45%).

¹H NMR (400 MHz, DMSO-d6) δ ppm: 10.34 (s, 2H), 7.56 (d, J=8.8 Hz, 1H), 7.27 (d, J=2.8 Hz, 1H), 7.01 (dd, J=9.2, 3.2 Hz, 1H), 4.69 (s, 2H), 4.15 (s, 2H).

LCMS Method: UPLC_2A, O2H METHOD_A_1, RT-1.986 min. MS: 311.1 (M+1).

UNI6-006

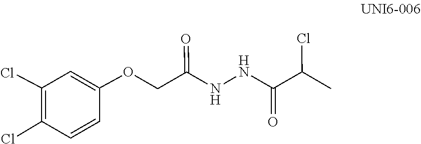 [SMILES: CC(Cl)C(=O)NNC(=O)COc1ccc(Cl)c(Cl)c1]

Route for UNI6-006

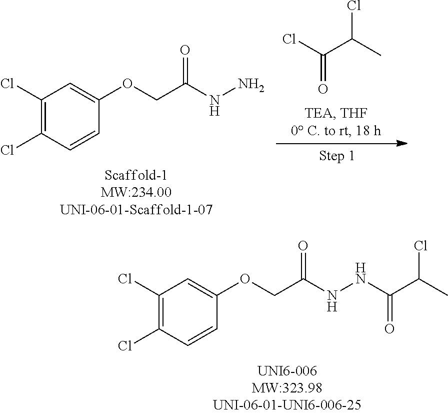

Step-1: Synthesis of 2-chloro-N′-(2-(3,4-dichlorophenoxy)acetyl)propanehydrazide

To a solution of Scaffold-1 (0.20 g, 0.000854 mol) in THF (2.0 mL) was added TEA (0.17 mL, 0.00128 mol) at 0° C. 2-Chloro propionyl chloride (0.08 mL, 0.000854 mol) was drop wise added to the reaction mixture at 0° C. The reaction mixture was stirred at 0° C. for 4 h and further at room temperature for 14 h. Reaction was monitored by TLC (5% methanol in chloroform). The resulting reaction mixture was poured into water (30 mL) and extracted with Ethyl acetate (2×25 mL). The combined organic phase was washed with brine solution (10 mL), dried over sodium sulfate, filtered and concentrated under vacuum. The obtained crude material was purified by flash chromatography (product was eluated using 50% Ethyl acetate in Hexane) yielding titled compound (0.047 g, 000145 mol, 17%).

¹H NMR (400 MHz, DMSO-d6) δ ppm: 10.37 (s, 1H), 10.35 (s, 1H), 7.56 (d, J=8.8 Hz, 1H), 7.26 (d, J=3.2 Hz, 1H), 7.01 (dd, J=8.8, 2.8 Hz, 1H), 4.69 (s, 2H), 4.56-4.55 (m, 1H), 1.55 (d, J=6.8 Hz, 3H).

LCMS Method: UPLC_2A, O2H METHOD_A_3, RT-2.133 min. MS: 323.31 (M−1).

### Example 4: Binding and Activity Data

TABLE 1
Binding and activity data for analogs of compound 87

| | | | | qPCR | qPCR | qPCR | qPCR | qPCR | qPCR |
|---|---|---|---|---|---|---|---|---|---|
| Compound | Binding | Binding | | MN9D | MN9D | MN9D | MN9D | SHSY5Y | SHSY5Y |
| number | MST | SPR | Luciferase | Pitx3 | Nurr1 | Th | Vmat2 | NURR1 | TH |
| SP 65 | + | + | − | − | − | − | − | nt | nt |
| SP 66 | − | − | − | nt | nt | nt | nt | nt | nt |
| SP 87 | +++ | + | ++++ | ++++ | ++++ | + | +++ | ++++ | ++ |
| SP 97 | − | − | − | − | − | − | − | nt | nt |
| SP 98 | + | + | − | − | − | − | − | nt | nt |
| SP 99 | + | + | − | − | − | − | − | nt | nt |
| SP 100 | − | + | − | − | + | − | − | nt | nt |
| SP 119 | − | − | − | nt | nt | nt | nt | nt | nt |
| SP 135 | − | − | − | − | − | − | − | nt | nt |
| SP 136 | + | + | − | nt | nt | nt | nt | nt | nt |
| SP 137 | − | − | − | nt | nt | nt | nt | nt | nt |
| SP 138 | + | + | − | nt | nt | nt | nt | nt | nt |
| SP 139 | − | − | − | nt | nt | nt | nt | nt | nt |
| SP 143 | − | − | − | − | + | − | + | nt | nt |
| SP 147 | − | + | − | − | − | − | − | nt | nt |
| SP 150 | − | − | − | − | − | − | − | nt | nt |
| SP 151 | − | − | + | ++++ | ++++ | + | ++ | nt | nt |
| SP 152 | − | − | − | − | − | − | − | nt | nt |
| SP 153 | +++ | +++ | + | ++++ | +++ | − | ++ | ++++ | +++ |
| SP 154 | − | − | − | − | − | − | − | nt | nt |
| SP 155 | + | + | − | + | − | − | − | nt | nt |
| SP 156 | ++ | ++ | +++ | ++++ | ++++ | ++++ | − | ++++ | +++ |
| SP 157 | +++ | + | +++ | ++++ | ++++ | + | +++ | ++++ | + |
| SP 158 | ++ | + | + | − | − | − | − | nt | nt |
| SP 159 | − | − | − | − | − | − | − | nt | nt |
| SP 162 | ++ | − | − | − | − | − | − | nt | nt |
| SP 163 | − | − | − | ++++ | ++++ | − | +++ | ++++ | ++ |
| SP 164 | − | + | − | ++++ | ++++ | + | +++ | ++++ | + |
| SP 166 | ++ | + | − | +++ | − | − | − | nt | nt |
| SP 168 | − | − | − | − | − | − | − | nt | nt |
| SP 169 | − | − | − | − | − | − | − | nt | nt |
| SP 170 | − | − | − | − | − | − | − | nt | nt |
| SP 171 | − | − | + | +++ | ++++ | + | ++++ | ++++ | + |
| SP 172 | − | − | − | − | − | − | − | nt | nt |
| SP 173 | − | − | +++ | ++++ | ++++ | ++++ | + | ++++ | ++ |
| SP 174 | − | + | − | + | +++ | − | − | nt | nt |
| SP 175 | − | − | − | − | − | − | − | nt | nt |
| SP 176 | − | − | − | − | − | − | − | nt | nt |
| SP 177 | − | − | − | + | − | − | − | nt | nt |
| SP 178 | + | − | − | + | − | − | − | nt | nt |
| SP 179 | − | − | − | − | − | − | − | nt | nt |
| SP 180 | − | − | − | − | − | − | − | nt | nt |
| SP 181 | − | − | − | − | − | − | − | nt | nt |
| SP 182 | − | − | − | − | − | − | − | nt | nt |
| SP 183 | + | + | + | + | − | − | − | nt | nt |
| SP 184 | − | − | − | ++++ | +++ | − | ++ | nt | nt |
| SP 186 | − | − | ++ | ++++ | ++++ | + | ++ | ++++ | + |
| SP 191 | ++ | + | − | ++++ | +++ | + | ++ | nt | nt |
| SP 193 | − | − | − | ++++ | ++++ | + | ++ | nt | nt |
| SP 194 | − | − | − | ++++ | +++ | + | ++ | nt | nt |
| SP 195 | − | − | − | ++ | ++ | + | + | nt | nt |
| SP 196 | − | − | − | + | +++ | ++ | + | nt | nt |
| GWL | + | − | nt | − | − | − | − | nt | nt |
| 2.137 | | | | | | | | | |
| GWL | − | + | nt | − | − | − | − | nt | nt |
| 2.129 | | | | | | | | | |
| GWL | − | + | nt | − | − | − | − | nt | nt |
| 2.154 | | | | | | | | | |
| GWL | − | + | nt | − | − | − | − | nt | nt |
| 2.131 | | | | | | | | | |
| GWL | + | + | nt | − | − | − | − | nt | nt |
| 3.008 | | | | | | | | | |
| GWL | − | − | nt | − | − | − | − | nt | nt |
| 2.97 | | | | | | | | | |
| GWL | − | − | nt | − | − | − | − | nt | nt |
| 2.143 | | | | | | | | | |
| GWL | − | − | nt | − | − | − | − | nt | nt |
| 2.171 | | | | | | | | | |
| GWL | − | − | nt | − | − | − | − | nt | nt |
| 2.190 | | | | | | | | | |
| GWL | − | + | nt | − | − | − | − | nt | nt |
| 3.001 | | | | | | | | | |

Key: Binding Assay: +++ = high nanomolar binding, ++ = low micromolar binding, += mid to high micromolar binding. Cellular Assay (cpds tested at 10 μM): +++++ => 10 fold, +++ =>4−fold, +++ = >3 fold, ++ => 1.5-fold. All Assays: nt = not tested.

TABLE 2
Binding and activity data for analogs of compound 85

| | | | | qPCR | qPCR | qPCR | qPCR |
|---|---|---|---|---|---|---|---|
| Compound | Binding | Binding | | MN9D | MN9D | MN9D | MN9D |
| number | MST | SPR | Luciferase | Pitx3 | Nurr1 | Th | Vmat2 |
| SP 43 | − | − | − | nt | nt | nt | nt |
| SP 44 | − | − | − | nt | nt | nt | nt |
| SP 45 | − | − | − | nt | nt | nt | nt |
| SP 46 | − | − | − | nt | nt | nt | nt |
| SP 47 | − | − | − | nt | nt | nt | nt |
| SP 51 | − | − | + | + | + | + | − |
| SP 52 | − | − | − | nt | nt | nt | nt |
| SP 53 | − | − | − | nt | nt | nt | nt |
| SP 54 | − | − | − | nt | nt | nt | nt |
| SP 55 | − | − | − | nt | nt | nt | nt |
| SP 56 | − | − | − | nt | nt | nt | nt |
| SP 59 | − | − | − | nt | nt | nt | nt |
| SP 60 | + | + | − | nt | nt | nt | nt |
| SP 61 | − | − | − | nt | nt | nt | nt |
| SP 62 | − | − | − | nt | nt | nt | nt |
| SP 64 | − | − | − | nt | nt | nt | nt |
| SP 85 | +++ | + | − | − | − | + | − |
| SP 94 | − | − | − | nt | nt | nt | nt |
| SP 95 | + | + | − | + | − | − | + |
| SP 96 | − | − | − | nt | nt | nt | nt |
| SP 102 | − | − | − | nt | nt | nt | nt |
| SP 108 | ++ | − | nt | − | + | + | − |
| SP 110 | − | − | − | nt | nt | nt | nt |
| SP 117 | − | − | − | nt | nt | nt | nt |
| SP 120 | − | − | − | nt | nt | nt | nt |
| SP 123 | − | − | + | nt | nt | nt | nt |
| SP 131 | − | − | − | nt | nt | nt | nt |
| SP 133 | − | − | − | nt | nt | nt | nt |
| SP 134 | − | − | − | nt | nt | nt | nt |
| SP 146 | − | + | − | nt | nt | nt | nt |
| UNI6-001 | + | nt | nt | nt | nt | nt | nt |
| UNI6-002 | ++ | nt | nt | nt | nt | nt | nt |
| UNI6-003 | + | nt | nt | + | − | + | − |
| UNI6-005 | ++ | nt | nt | nt | nt | nt | nt |
| UNI6-006 | ++ | nt | nt | nt | nt | nt | nt |

Key: high
Binding Assay: +++ = high nanomolar binding, +++ = low micromolar binding, + = mid to micromolar binding. Cellular Assay (cpds tested at 10 μM): ++++ => 10 fold, +++ => 4-fold, +++ => 3 fold, ++ => 1.5-fold. All Assays: nt = not tested.

### Example 5: Additional Compounds

TABLE 3
Additional compounds

| | |
|---|---|
| 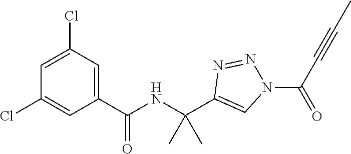 [SMILES: CC#CC(=O)n1cc(C(C)(C)NC(=O)c2cc(Cl)cc(Cl)c2)nn1] | 160 |
| 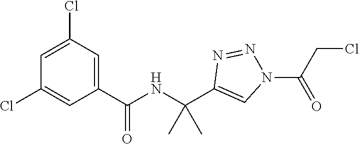 [SMILES: CC(C)(NC(=O)c1cc(Cl)cc(Cl)c1)c1cn(C(=O)CCl)nn1] | 161 |
| 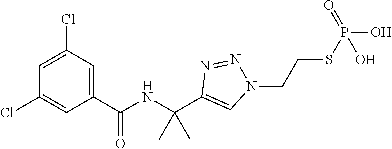 [SMILES: CC(C)(NC(=O)c1cc(Cl)cc(Cl)c1)c1cn(CCSP(=O)(O)O)nn1] | 165 |
| 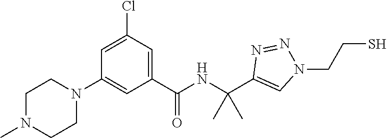 [SMILES: CN1CCN(c2cc(Cl)cc(C(=O)NC(C)(C)c3cn(CCS)nn3)c2)CC1] | 167 |

### Example 6: Nurr1 Cancer Indications

Nurr1 is involved in multiple biological processes that are important in tumorigenesis, such as cell proliferation, apoptosis, differentiation, and glucose utilization. Nurr1 is a potential cancer therapeutic (Komiya et al., 2017). Specific cancers in which Nurr1 (NR⁴A2) expression and/or activity plays a role include: breast cancer (Llopis et al., 2013), pancreatic cancer (Li et al., 2012), bladder cancer (Inamoto et al., 2010; Inamoto et al., 2008), mucoepidermoid carcinoma (MEC), gastric cancer (Guo et al., 2015), prostate cancer (Wang et al., 2013), colorectal cancer (Han et al, 2013; Vijaykumar et al., 2005), lung cancer (Bai et al, 2015), adrenocortical cancer (Li et al, 2018), and cervical cancer (Li et al., 2006).

### REFERENCES

1. Alavian, K. N., Jeddi, S., Naghipour, S. I., Nabili, P., Licznerski, P., and Tierney, T. S. (2014). The lifelong maintenance of mesencephalic dopaminergic neurons by Nurr1 and engrailed. J Biomed Sci 21, 27. 2. Blesa, J., Trigo-Damas, I., Quiroga-Varela, A., and Jackson-Lewis, V. R. (2015). Oxidative stress and Parkinson's disease. Front Neuroanat 9, 91. 3. Campos-Melo, D., Galleguillos, D., Sanchez, N., Gysling, K., and Andres, M. E. (2013). Nur transcription factors in stress and addiction. Front Mol Neurosci 6, 44. 4. Chu, Y., Le, W., Kompoliti, K., Jankovic, J., Mufson, E. J., and Kordower, J. H. (2006). Nurr1 in Parkinson's disease and related disorders. J Comp Neurol 494, 495-514. 5. de Lau, L. M., and Breteler, M. M. (2006). Epidemiology of Parkinson's disease. Lancet Neurol 5, 525-535. 6. Decressac, M., Volakakis, N., Bjorklund, A., and Perlmann, T. (2013). NURR1 in Parkinson disease from pathogenesis to therapeutic potential. Nat Rev Neurol 9, 629-636. 7. Dong, J., Li, S., Mo, J. L., Cai, H. B., and Le, W. D. (2016). Nurr1-Based Therapies for Parkinson's Disease. CNS Neurosci Ther 22, 351-359. 8. Galleguillos, D., Fuentealba, J. A., Gomez, L. M., Saver, M., Gomez, A., Nash, K., Burger, C., Gysling, K., and Andres, M. E. (2010). Nurr1 regulates RET expression in dopamine neurons of adult rat midbrain. J Neurochem 114, 1158-1167. 9. Grimes, D. A., Han, F., Panisset, M., Racacho, L., Xiao, F., Zou, R., Westaff, K., and Bulman, D. E. (2006). Translated mutation in the Nurr1 gene as a cause for Parkinson's disease. Mov Disord 21, 906-909. 10. Hauser, D. N., and Hastings, T. G. (2013). Mitochondrial dysfunction and oxidative stress in Parkinson's disease and monogenic parkinsonism. Neurobiol Dis 51, 35-42. 11. Hermanson, E., Joseph, B., Castro, D., Lindqvist, E., Aarnisalo, P., Wallen, A., Benoit, G., Hengerer, B., Olson, L., and Perlmann, T. (2003). Nurr1 regulates dopamine synthesis and storage in MN9D dopamine cells. Exp Cell Res 288, 324-334. 12. Hwang, O. (2013). Role of oxidative stress in Parkinson's disease. Exp Neurobiol 22, 11-17. 13. Iwawaki, T., Kohno, K., and Kobayashi, K. (2000). Identification of a potential nurrl response element that activates the tyrosine hydroxylase gene promoter in cultured cells. Biochem Biophys Res Commun 274, 590-595. 14. Janezic, S., Threlfell, S., Dodson, P. D., Dowie, M. J., Taylor, T. N., Potgieter, D., Parkkinen, L., Senior, S. L., Anwar, S., Ryan, B., et al. (2013). Deficits in dopaminergic transmission precede neuron loss and dysfunction in a new Parkinson model. Proc Natl Acad SciUSA 110, E4016-4025. 15. Jankovic, J., Chen, S., and Le, W. D. (2005). The role of Nurr1 in the development of dopaminergic neurons and Parkinson's disease. Prog Neurobiol 77, 128-138. 16. Jiang, C., Wan, X., He, Y., Pan, T., Jankovic, J., and Le, W. (2005). Age-dependent dopaminergic dysfunction in Nurr1 knockout mice. Exp Neurol 191, 154-162. 17. Johnson, M. M., Michelhaugh, S. K., Bouhamdan, M., Schmidt, C. J., and Bannon, M. J. (2011). The Transcription Factor NURR1 Exerts Concentration-Dependent Effects on Target Genes Mediating Distinct Biological Processes. Front Neurosci 5, 135. 18. Kadkhodaei, B., Alvarsson, A., Schintu, N., Ramskold, D., Volakakis, N., Joodmardi, E., Yoshitake, T., Kehr, J., Decressac, M., Bjorklund, A., et al. (2013). Transcription factor Nurr1 maintains fiber integrity and nuclear-encoded mitochondrial gene expression in dopamine neurons. Proc Natl Acad Sci USA 110, 2360-2365. 19. Kadkhodaei, B., Ito, T., Joodmardi, E., Mattsson, B., Rouillard, C., Carta, M., Muramatsu, S., Sumi-Ichinose, C., Nomura, T., Metzger, D., et al. (2009). Nurr1 is required for maintenance of maturing and adult midbrain dopamine neurons. J Neurosci 29, 15923-15932. 20. Kim, C. H., Han, B. S., Moon, J., Kim, D. J., Shin, J., Rajan, S., Nguyen, Q. T., Sohn, M., Kim, W. G., Han, M., et al. (2015). Nuclear receptor Nurr1 agonists enhance its dual functions and improve behavioral deficits in an animal model of Parkinson's disease. Proc Natl Acad Sci USA 112, 8756-8761. 21. Kim, K. S., Kim, C. H., Hwang, D. Y., Seo, H., Chung, S., Hong, S. J., Lim, J. K., Anderson, T., and Isacson, O. (2003). Orphan nuclear receptor Nurr1 directly transactivates the promoter activity of the tyrosine hydroxylase gene in a cell-specific manner. J Neurochem 85, 622-634. 22. Le, W., Pan, T., Huang, M., Xu, P., Xie, W., Zhu, W., Zhang, X., Deng, H., and Jankovic, J. (2008). Decreased NURR1 gene expression in patients with Parkinson's disease. J Neurol Sci 273, 29-33. 23. Le, W. D., Xu, P., Jankovic, J., Jiang, H., Appel, S. H., Smith, R. G., and Vassilatis, D. K. (2003). Mutations in NR4A2 associated with familial Parkinson disease. Nat Genet 33, 85-89. 24. Luo, Y. (2012). The function and mechanisms of Nurr1 action in midbrain dopaminergic neurons, from development and maintenance to survival. Int Rev Neurobiol 102, 1-22. 25. Montarolo, F., Perga, S., Martire, S., Navone, D. N., Marchet, A., Leotta, D., and Bertolotto, A. (2016). Altered NR4A Subfamily Gene Expression Level in Peripheral Blood of Parkinson's and Alzheimer's Disease Patients. Neurotox Res 30, 338-344. 26. Moon, M., Jung, E. S., Jeon, S. G., Cha, M. Y., Jang, Y., Kim, W., Lopes, C., Mook-Jung, I., and Kim, K. S. (2018). Nurr1 (NR4A2) regulates Alzheimer's disease-related pathogenesis and cognitive function in the SXFAD mouse model. Aging Cell, e12866. 27. Moran, L. B., Croisier, E., Duke, D. C., Kalaitzakis, M. E., Roncaroli, F., Deprez, M., Dexter, D. T., Pearce, R. K., and Graeber, M. B. (2007). Analysis of alpha-synuclein, dopamine and parkin pathways in neuropathologically confirmed parkinsonian nigra. Acta Neuropathol 113, 253-263. 28. Sacchetti, P., Mitchell, T. R., Granneman, J. G., and Bannon, M. J. (2001). Nurr1 enhances transcription of the human dopamine transporter gene through a novel mechanism. J Neurochem 76, 1565-1572. 29. Saijo, K., Winner, B., Carson, C. T., Collier, J. G., Boyer, L., Rosenfeld, M. G., Gage, F. H., and Glass, C. K. (2009). A Nurr1/CoREST pathway in microglia and astrocytes protects dopaminergic neurons from inflammation-induced death. Cell 137, 47-59. 30. Sakurada, K., Ohshima-Sakurada, M., Palmer, T. D., and Gage, F. H. (1999). Nurr1, an orphan nuclear receptor, is a transcriptional activator of endogenous tyrosine hydroxylase in neural progenitor cells derived from the adult brain. Development 126, 4017-4026. 31. Samii, A., Nutt, J. G., and Ransom, B. R. (2004). Parkinson's disease. Lancet 363, 1783-1793. 32. Volpicelli, F., Caiazzo, M., Greco, D., Consales, C., Leone, L., Perrone-Capano, C., Colucci D′Amato, L., and di Porzio, U. (2007). Bdnf gene is a downstream target of Nurr1 transcription factor in rat midbrain neurons in vitro. J Neurochem 102, 441-453. 33. Wang, Z., Benoit, G., Liu, J., Prasad, S., Aarnisalo, P., Liu, X., Xu, H., Walker, N. P., and Perlmann, T. (2003). Structure and function of Nurr1 identifies a class of ligand-independent nuclear receptors. Nature 423, 555-560. 34. Wirdefeldt, K., Adami, H. O., Cole, P., Trichopoulos, D., and Mandel, J. (2011). Epidemiology and etiology of Parkinson's disease: a review of the evidence. Eur J Epidemiol 26 Suppl 1, S1-58. 35. Zetterstrom, R. H., Solomin, L., Jansson, L., Hoffer, B. J., Olson, L., and Perlmann, T. (1997). Dopamine neuron agenesis in Nurr1-deficient mice. Science 276, 248-250. 36. Zhang, L., Le, W., Xie, W., and Dani, J. A. (2012). Age-related changes in dopamine signaling in Nurr1 deficient mice as a model of Parkinson's disease. Neurobiol Aging 33, 1001 e1007-1016. 37. Gronemeyer, H., Gustafsson, J. A., and Laudet, V. (2004) Principles for modulation of the nuclear receptor superfamily, Nat Rev Drug Discov 3, 950-964. 38. Dubois, C., Hengerer, B., and Mattes, H. (2006) Identification of a potent agonist of the orphan nuclear receptor Nurr1, ChemMedChem 1, 955-958. 39. Smith, G. A., Rocha, E. M., Rooney, T., Barneoud, P., McLean, J. R., Beagan, J., Osborn, T., Coimbra, M., Luo, Y., Hallett, P. J., and Isacson, O. (2015) A Nurr1 agonist causes neuroprotection in a Parkinson's disease lesion model primed with the toll-like receptor 3 dsRNA inflammatory stimulant poly(I:C), PLoS One 10, e0121072. 40. Zhang, Z., Li, X., Xie, W. J., Tuo, H., Hintermann, S., Jankovic, J., and Le, W. (2012) Anti-parkinsonian effects of Nurr1 activator in ubiquitin-proteasome system impairment induced animal model of Parkinson's disease, CNS Neurol Disord Drug Targets 11, 768-773. 41. McFarland, K., Spalding, T. A., Hubbard, D., Ma, J. N., Olsson, R., and Burstein, E. S. (2013) Low dose bexarotene treatment rescues dopamine neurons and restores behavioral function in models of Parkinson's disease, ACS Chem Neurosci 4, 1430-1438. 42. Poppe, L., Harvey, T. S., Mohr, C., Zondlo, J., Tegley, C. M., Nuanmanee, O., and Cheetham, J. (2007) Discovery of ligands for Nurr1 by combined use of NMR screening with different isotopic and spin-labeling strategies, J Biomol Screen 12, 301-311. 43. Spathis, A. D., Asvos, X., Ziavra, D., Karampelas, T., Topouzis, S., Cournia, Z., Qing, X., Alexakos, P., Smits, L. M., Dalla, C., Rideout, H. J., Schwamborn, J. C., Tamvakopoulos, C., Fokas, D., and Vassilatis, D. K. (2017) Nurr1:RXRalpha heterodimer activation as monotherapy for Parkinson's disease, Proc Natl Acad SciUSA 114, 3999-4004. 44. Volakakis, N., Tiklova, K., Decressac, M., Papathanou, M., Mattsson, B., Gillberg, L., Nobre, A., Bjorklund, A., and Perlmann, T. (2015) Nurr1 and Retinoid X Receptor Ligands Stimulate Ret Signaling in Dopamine Neurons and Can Alleviate alpha-Synuclein Disrupted Gene Expression, J Neurosci 35, 14370-14385. 45. Perlmann, T., and Jansson, L. (1995) A novel pathway for vitamin A signaling mediated by RXR heterodimerization with NGFI-B and NURR1, Genes Dev 9, 769-782. 46. Zhan, Y. Y., Chen, Y., Zhang, Q., Zhuang, J. J., Tian, M., Chen, H. Z., Zhang, L. R., Zhang, H. K., He, J. P., Wang, W. J., Wu, R., Wang, Y., Shi, C., Yang, K., Li, A. Z., Xin, Y. Z., Li, T. Y., Yang, J. Y., Zheng, Z. H., Yu, C. D., Lin, S. C., Chang, C., Huang, P. Q., Lin, T., and Wu, Q. (2012) The orphan nuclear receptor Nur77 regulates LKB1 localization and activates AMPK, Nat Chem Biol 8, 897-904. 47. Li, L., Liu, Y., Chen, H. Z., Li, F. W., Wu, J. F., Zhang, H. K., He, J. P., Xing, Y. Z., Chen, Y., Wang, W. J., Tian, X. Y., Li, A. Z., Zhang, Q., Huang, P. Q., Han, J., Lin, T., and Wu, Q. (2015) Impeding the interaction between Nur77 and p38 reduces LPS--induced inflammation, Nat Chem Biol 11, 339-346. 48. Bogan, A. A., and Thorn, K. S. (1998) Anatomy of hot spots in protein interfaces, J Mol Biol 280, 1-9. 49. Krissinel, E., and Henrick, K. (2007) Inference of macromolecular assemblies from crystalline state, J Mol Biol 372, 774-797. 50. Khorasanizadeh, S., and Rastinejad, F. (2016) Visualizing the Architectures and Interactions of Nuclear Receptors, Endocrinology 157, 4212-4221. 51. Chandra, V., Huang, P., Hamuro, Y., Raghuram, S., Wang, Y., Burris, T. P., and Rastinejad, F. (2008) Structure of the intact PPAR—gamma—RXR—nuclear receptor complex on DNA, Nature 456, 350-356. 52. Lou, X., Toresson, G., Benod, C., Suh, J. H., Philips, K. J., Webb, P., and Gustafsson, J. A. (2014) Structure of the retinoid X receptor alpha-liver X receptor beta (RXRalpha-LXRbeta) heterodimer on DNA, Nat Struct Mol Biol 21, 277-281. 53. Meiser, J., Weindl, D., and Hiller, K. (2013) Complexity of dopamine metabolism, Cell Commun Signal 11, 34. 54. d'Ischia, M., Napolitano, A., Pezzella, A., Land, E. J., Ramsden, C. A., and Riley, P. A. (2005) 5,6-Dihydroxyindoles and Indole-5,6-diones, Advances in Heterocyclic Chemistry 89, 1-55. 55. Pezzella, A., Iadonisi, A., Valerio, S., Panzella, L., Napolitano, A., Adinolfi, M., and d'Ischia, M. (2009) Disentangling eumelanin “black chromophore”: visible absorption changes as signatures of oxidation stateand aggregation-dependent dynamic interactions in a model water-soluble 5,6-dihydroxyindole polymer, J Am Chem Soc 131, 15270-15275. 56. Sacchetti, P., Dwornik, H., Formstecher, P., Rachez, C., and Lefebvre, P. (2002) Requirements for heterodimerization between the orphan nuclear receptor Nurr1 and retinoid X receptors, J Biol Chem 277, 35088-35096. 57. Wallen-Mackenzie, A., Mata de Urquiza, A., Petersson, S., Rodriguez, F. J., Friling, S., Wagner, J., Ordentlich, P., Lengqvist, J., Heyman, R. A., Arenas, E., and Perlmann, T. (2003) Nurr1-RXR heterodimers mediate RXR ligand-induced signaling in neuronal cells, Genes Dev 17, 3036-3047. 58. Lammi, J., Huppunen, J., and Aarnisalo, P. (2004) Regulation of the osteopontin gene by the orphan nuclear receptor NURR1 in osteoblasts, Mol Endocrinol 18, 1546-1557. 59. Castro, D. S., Hermanson, E., Joseph, B., Wallen, A., Aarnisalo, P., Heller, A., and Perlmann, T. (2001) Induction of cell cycle arrest and morphological differentiation by Nurr1 and retinoids in dopamine MN9D cells, J Biol Chem 276, 43277-43284. 60. Maira, M., Martens, C., Batsche, E., Gauthier, Y., and Drouin, J. (2003) Dimer-specific potentiation of NGFI-B (Nur77) transcriptional activity by the protein kinase A pathway and AF-1-dependent coactivator recruitment, Mol Cell Biol 23, 763-776. 61. Maira, M., Martens, C., Philips, A., and Drouin, J. (1999) Heterodimerization between members of the Nur subfamily of orphan nuclear receptors as a novel mechanism for gene activation, Mol Cell Biol 19, 7549-7557. 62. Komiya, T., Yamamoto, S., Roy, A., McDonald, P., Perez, R. P. (2017) Drug screening to target nuclear orphan receptor NR4A2 for cancer therapeutics. Transl Lung Cancer Res. 6(5): 600-610. 63. Llopis et al. BMC Cancer 2013, 13:139. 64. Li, X., Lee, S. O., Safe, S. (2012) Structure-dependent activation of NR4A2 (Nurr1) by 1,1-bis(3′-indolyl)-1-(aromatic)methane analogs in pancreatic cancer cells. Biochem Pharmacol. 83(10): 1445-55. 65. Inamoto, T., Czerniak, B. A., Dinney, C. P., Kamat, A. M. (2010) Cytoplasmic mislocalization of the orphan nuclear receptor Nurr1 is a prognostic factor in bladder cancer. Cancer 116: 340-6. 66. Inamoto, T., Papineni, S., Chintharlapalli, S., et al. (2008) 1,1-Bis(3′-indolyl)-1-(p-chlorophenyl)methane activates the orphan nuclear receptor Nurr1 and inhibits bladder cancer growth. Mol Cancer Ther 7: 3825-33. 67. Guo, J., Zu, G., Zhou, T., et al. (2015) Clinicopathological significance of orphan nuclear receptor Nurr1 expression in gastric cancer. Clin Transl Oncol 17: 788-94. 68. Wang, J., Yang, J., Zou, Y., et al. (2013) Orphan nuclear receptor nurrl as a potential novel marker for progression in human prostate cancer. Asian Pac J Cancer Prey 14:2023-8. 69. Han, Y., Cai, H., Ma, L., et al. (2013) Nuclear orphan receptor NR4A2 confers chemoresistance and predicts unfavorable prognosis of colorectal carcinoma patients who received postoperative chemotherapy, Eur J Cancer 49: 3420-30. 70. Holla, V. R., Mann, J. R., Shi, Q., and DuBois, R. N. (2005) J Biol Chem 281, 2676-2682. 71. Bai, R., Weng, C., Dong, H., Li, S., Chen, G., Xu, Z. (2015) MicroRNA-409-3p suppresses colorectal cancer invasion and metastasis partly by targeting GAB1 expression. Int J Cancer. 137(10): 2310-22. 72. Li, X., Wang, B., Tang, L., Zhang, Y., Chen, L., Gu, L., Zhang, F., Ouyang, J., Zhang, X. (2018) GSTA1 Expression Is Correlated With Aldosterone Level in KCNJ5-Mutated Adrenal Aldosterone-Producing Adenoma. J Clin Endocrinol Metab. 103(3): 813-823. 73. Li, Q.-X., Ke, N., Sundaram, R., Wong-Staal, F. (2006) NR4A1, 2, 3—an orphan nuclear hormone receptor family involved in cell apoptosis and carcinogenesis. Histol Histopathol. 21(5): 533-40. 74. Bruning, J. M., Wang, Y., Oltrabella, F., Tian, B., Kholodar, S. A., Liu, H., Bhattacharya, P., Guo, S., Holton, J. M., Fletterick, R. J., Jacobson, M. P., England, P. A. (2019) Covalent modification and regulation of the nuclear receptor Nurr1 by a dopamine metabolite. Cell Chem. Biol. 26(5): 674-685.

1. (canceled)

2. A compound, having the formula 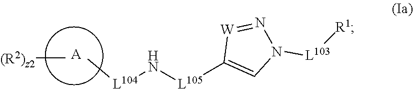
wherein
Ring A is aryl or heteroaryl;
L¹⁰⁴ is a bond, —S(O)₂—, —C(O)—, —NHC(O)—, —OC(O)—, substituted or unsubstituted alkylene, or substituted or unsubstituted heteroalkylene;
L¹⁰⁵ is a bond, substituted or unsubstituted alkylene, substituted or unsubstituted heteroalkylene, substituted or unsubstituted cycloalkylene, or substituted or unsubstituted heterocycloalkylene;
L¹⁰³ is a bond or substituted or unsubstituted alkylene
W is N or CH;
R¹ is —OCX¹₃, —OCH₂X¹, —OCHX¹₂, —N(O)ₘ₁, —SC(O)R^{1C}, —SH, —SSCH₃, —SeH, —NR^{1A}OR^{1C}, —SP(O)(OH)₂, or E;
E is an electrophilic moiety;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1C}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n2 is independently an integer from 0 to 4;
m1, m2, and v2 are independently 1 or 2;
X¹ and X² are independently —F, —Cl, —Br, or —I; and
z2 is an integer from 0 to 5.

3. (canceled)

4. The compound of claim 2, wherein Ring A is a phenyl.

5. (canceled)

6. The compound of claim 2, wherein the compound has the formula 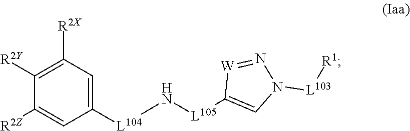
and
R^{2X}, R^{2Y}, and R^{2Z} are independently hydrogen, halogen, —CX²₃, —CHX²₂, —CH₂X², —OCX²₃, —OCH₂X², —OCHX²₂, —CN, —SOₙ₂R^{2D}, —SO₂ᵥNR^{2A}R^{2B}, —NHC(O)NR^{2A}R^{2B}, —N(O)^{m2}, —NR^{2A}R^{2B}, —C(O)R^{2C}, —C(O)OR^{2C}, —C(O)NR^{2A}R^{2B}, —OR^{2D}, —NR^{2A}SO₂R^{2D}, —NR^{2A}C(O)R^{2C}, —NR^{2A}C(O)_{OR}^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2X} and R^{2Y} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl; R^{2Y} and R^{2Z} substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl.

7. The compound of claim 6, wherein
R^{2X} is halogen;
R^{2Y} is hydrogen; and
R^{2Z} is halogen.

8.-10. (canceled)

11. The compound of claim 2, wherein L¹⁰⁴ is —C(O)—.

12. (canceled)

13. The compound of claim 2, wherein L¹⁰⁵ is an unsubstituted C₁-C₄ alkylene.

14. The compound of claim 2, wherein L¹⁰⁵ is

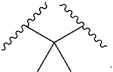 [SMILES: *C(*)(C)C]

15.-18. (canceled)

19. The compound of claim 2, wherein

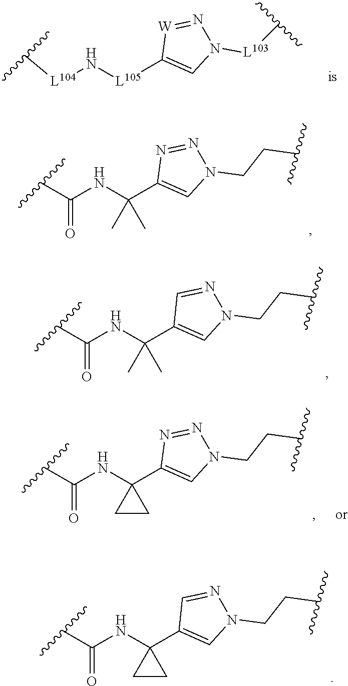

20.-22. (canceled)

23. The compound of claim 2, wherein R¹ is —SH, —SC(O)CH₃, or —SSCH₃.

24. The compound of claim 2, having the formula:

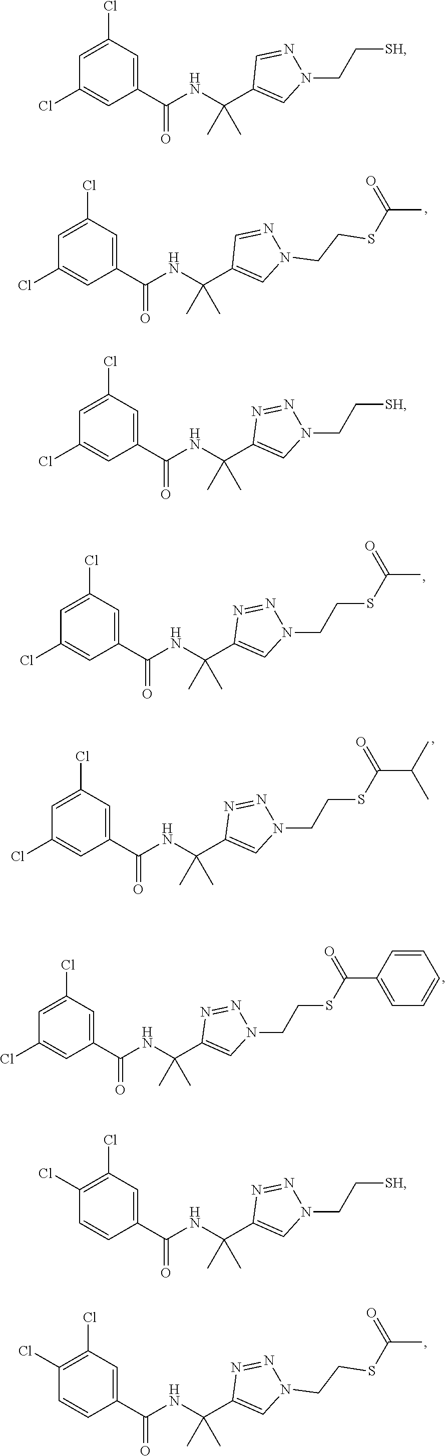

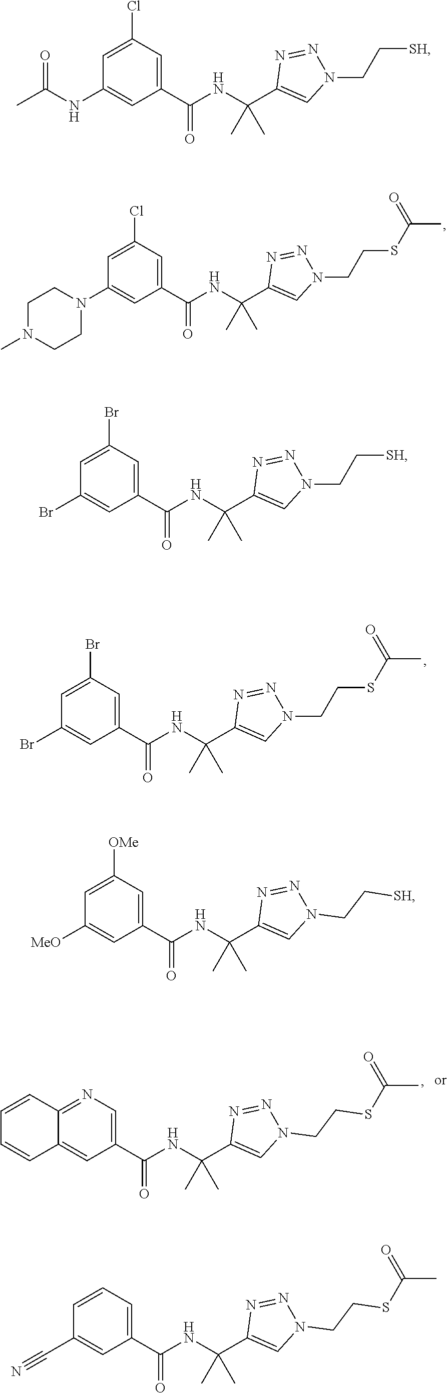

25.-27. (canceled)

28. A compound, the formula 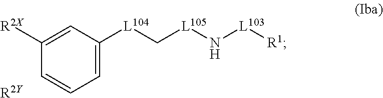
L¹⁰⁴ is —O;
L¹⁰⁵ is —S(O)₂—, —C(O)—, —NHC(O)—, or —OC(O)—;
L103 is unsubstituted alkylene;
R¹ is halogen, —CX¹₃, —CHX¹₂, —CH₂X¹, —OCX¹₃, —OCH₂X¹, —OCHX¹₂, —CN, —SOₙ₁R^{1D}, —SOᵥ₁R^{1A}R^{1B}, —NHC(O)NR^{1A}R^{1B}, —N(O)ₘ₁, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)OR^{1C}, —NR^{1A}OR^{1C}, —N₃, —SSR^{1D}, —SiR^{1A}R^{1B}R^{1C}, —SP(O)(OH)₂, E, or substituted or unsubstituted cycloalkyl;
E is an electrophilic moiety;
R^{2X} and R^{2Y} are independently halogen, —OCX²₃, —OCH₂X², —OCHX²₂, —SOₙ₂R^{2D}, —SOᵥ₂NR^{2A}R^{2B}, —NHC(O)NR^{2A}R^{2B}, —N(O)ₘ₂, —OR^{2D}, —NR^{2A}SO₂R^{2D}, —NR^{2A}C(O)OR^{2C}, —NR^{2A}OR^{2C}, —N₃, substituted or unsubstituted cycloalkyl, substituted or unsubstituted heterocycloalkyl, or substituted or unsubstituted aryl;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1, and v2 are independently 1 or 2; and
X¹ and X² are independently —F, —Cl, —Br, or —I.

29. The compound of claim 28, wherein R^{2X} and R^{2Y} are independently halogen.

30. (canceled)

31. (canceled)

32. The compound of claim 28, wherein L¹⁰⁵ is —C(O)—.

33.-36. (canceled)

37. The compound of claim 28,
wherein -L¹⁰⁴-CH₂-L¹⁰⁵-NH-L¹⁰³—is

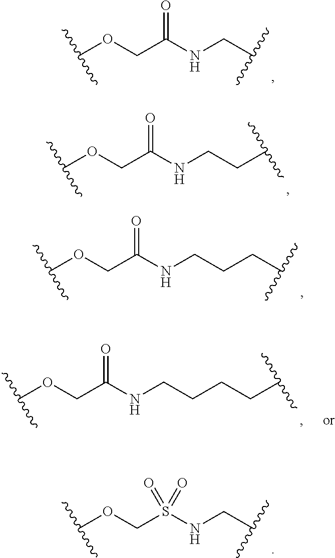 [SMILES: *CNC(=O)CO* ; *CCNC(=O)CO* ; *CCCNC(=O)CO* ; *CCCCNC(=O)CO* ; *CNS(=O)(=O)CO*]

38. The compound of claim 28, wherein
R¹ is —SH, —SC(O)CH₃ or —SSCH₃.

39. (canceled)

40. (canceled)

41. The compound of claim 28, having the formula:

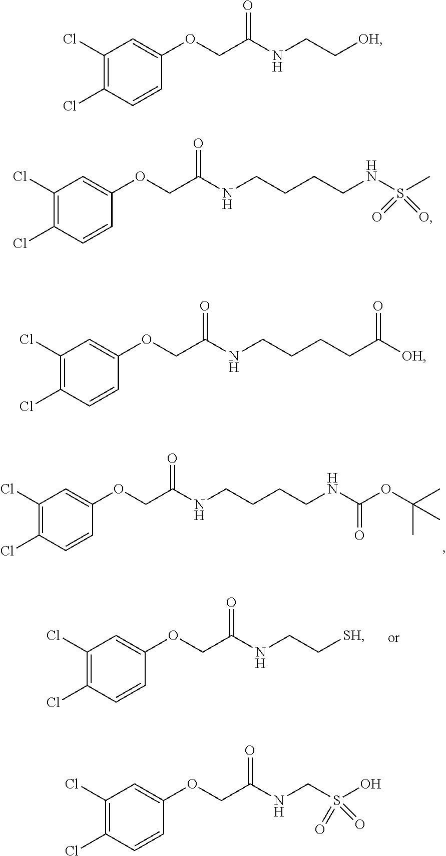 [SMILES: O=C(COc1ccc(Cl)c(Cl)c1)NCCO ; CS(=O)(=O)NCCCCNC(=O)COc1ccc(Cl)c(Cl)c1 ; O=C(O)CCCCNC(=O)COc1ccc(Cl)c(Cl)c1 ; CC(C)(C)OC(=O)NCCCCNC(=O)COc1ccc(Cl)c(Cl)c1 ; O=C(COc1ccc(Cl)c(Cl)c1)NCCS ; O=C(COc1ccc(Cl)c(Cl)c1)NCS(=O)(=O)O]

42. A pharmaceutical composition comprising a pharmaceutically acceptable excipient and a compound having the formula: 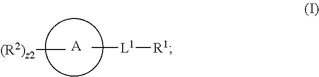
wherein
Ring A is aryl or heteroaryl;
L¹ is L¹⁰¹-L¹⁰²-L¹⁰²-L¹⁰³;
L¹⁰¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, L¹⁰⁴-L¹⁰⁵, L¹⁰⁴-NH-L¹⁰⁵, or L¹⁰⁴-CH₂-L¹⁰⁵;
L¹⁰² is a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —O—, —NH—, —S—, —S(O)₂—, —C(O)—, —NHC(O)—, —C(O)NH—, —OC(O)—, —C(O)O—, substituted or unsubstituted alkylene, or substituted or unsubstituted heteroalkylene;
L¹⁰⁵ is a bond, —O—, —NH—, —S—, —S(O)₂—, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene;
R¹⁰¹, R¹⁰², and R¹⁰³ are independently hydrogen, halogen, —CCl₃, —CBr₃, —CF₃. —CI₃, —CH₂Cl, —CH₂Br, —CH₂F, —CH₂I, —CHCl₂, —CHBr₂, —CHF₂, —CHI₂, —CN, —OH, —NH, —COOH, —CONH₂, —NO₂, —SH, —SO₃H, —SO₄H, —SO₂NH₂, —NHNH₂, —ONH₂, —NHC(O)NHNH₂, —NHC(O)NH, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl;
R¹ is hydrogen, halogen, —CX¹₃, —CHX¹₂, —CH₂X¹, —OCX¹₃, —OCH₂X¹, —OCHX¹₂, —CN, —SOᵥ₁R^{1A}R^{1D}, —NHC(O)NR^{1A}R^{1D}, —N(O)ₙo, —NR^{1A}R^{1D}, —C(O)R^{K}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —SSR^{1D},—SiR^{1A}R^{1D}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
R² is independently halogen, —CX², —CHX²₁, —CH₂X², —OCX²_{3.}, —OCH₂X², —OCHX²₁, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1, and v2 are independently 1 or 2;
X¹ and X² are independently —F, —Cl, —Br, or —I; and
z2 is an integer from 0 to 5.

43. A method for treating a disease associated with dysregulation and/or degeneration of dopaminergic neurons in the central nervous system of a subject in need thereof, said method comprising administering to the subject in need thereof a therapeutically effective amount of a compound having the formula: 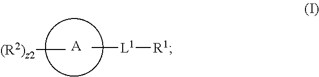
wherein
Ring A is aryl or heteroaryl;
L¹ ios L¹⁰¹-L¹⁰²-L¹⁰³;
L¹⁰¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, L¹⁰⁴-L¹⁰⁵, L¹⁰⁴-NH-L¹⁰⁵, or L¹⁰⁴-CH₂-L¹⁰⁵;
L¹⁰² is a bond, —S(O)—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —O—, —NH—, —S—, —S(O)—, —C(O)—, —NHC(O)—, —C(O)NH—, —OC(O)—, —C(O)O—, substituted or unsubstituted alkylene, or substituted or unsubstituted heteroalkylene;
L¹⁰⁵ is a bond, —O—, —NH—, —S—, —S(O)—, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene;
R¹⁰¹, R¹⁰², and R¹⁰³ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃ —, —OCH₂Cl, —OCH₂Br, —OCH₂F, —OCH₂I, —OCHCl_{2.}, —OCHBr₃, —OCHF₃, unsubstituted alkyl, unsubstituted heteroalkyl, unsubstituted cycloalkyl, unsubstituted heterocycloalkyl, unsubstituted aryl, or unsubstituted heteroaryl;
R¹ is hydrogen, halogen, —CX¹₃, —CHX¹₂, —CH₂X¹, —OCX¹₃ —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SSR^{1D},—SiR^{1A}R^{1B}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
R² is independently halogen, —CX²₃ —CHX²₂, —CH₂X², —OCX²₃ —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl. substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHD₂, —CHI₂, —CN, —OH, —NH₂, —COOH, —CONH₂, —NO,₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl; R^{2A}and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1, and v2 are independently 1 or 2;
X¹ and X² are independently —F, —Cl, —Br, or —I; and
z2 is an integer from 0 to 5.

44. The method of claim 43, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

45. (canceled)

46. A method of treating a cancer in a subject in need thereof, the method comprising administering to the subject in need thereof a therapeutically effective amount of a compound having the formula: 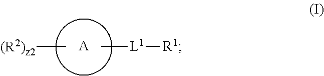
wherein
Ring A is aryl or heteroaryl;
L¹ is L¹⁰¹-L¹⁰²-L¹⁰³;
L¹⁰¹ is a bond, —S(O)₂, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, L¹⁰⁴-L¹⁰⁵, L¹⁰⁴-NH-L¹⁰⁵, or L¹⁰⁴-CH₂-L¹⁰⁵;
L¹⁰² is a bond, —S(O)₂13 , —N(R¹⁰²)—, —O—, —S—, —C(O)—, —C(O)N(R¹⁰³)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —O—, —NH—, —S—, —S(O)₂, —C(O)—, —NHC(O)—, —C(O)NH—, —OC(O)—, —C(O)O—, substituted or unsubstituted alkylene, or substituted or unsubstituted heteroalkylene;
L¹⁰⁵ is a bond, —O—, —NH—, —S—, —S(O)₂, —C(O)—, —NHC(O)—, —C(O)NH—, —OC(O)—, —C(O)O—, substituted or unsubstituted alkylene, substituted or unsubstituted heteroalkylene, substituted or unsubstituted cycloalkylene, or substituted or unsubstituted heterocycloalkylene;
R¹⁰¹, R¹⁰², and R¹⁰³ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁R^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SSR^{1D},—SiR^{1A}R^{1B}R^{1C}, —SP(O)(OH)₂, E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{1C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents bonded to adjacent atoms may be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1 B} substituents bonded to the same nitrogen atom may be joined to form a substituted or unsubstituted heterocycloalkyl or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl;
n1 and n2 are independently an integer from 0 to 4;
m1, m2, v1, and v2 are independently 1 or 2;
X¹ and X² are independently —F, —Cl, —Br, or —I; and
z2 is an integer from 0 to 5.

47.-55. (canceled)
